(19) United States
(12) Patent Application Publication
(10) Pub. No.: US 2011/0020865 A1
(43) Pub. Date: Jan. 27, 2011

(54) Gene Expression Technique

(75) Inventors: Thomas Payne, Nottingham (GB); Darrell Sleep, Notitngham (GB); Christopher John Arthur Finnis, Nottingham (GB); Leslie Robert Evans, Nottingham (GB)

Correspondence Address: NOVOZYMES NORTH AMERICA, INC., 500 FIFTH AVENUE, SUITE 1600, NEW YORK, NY, 10110, US

(73) Assignees: Novozymes Delta Limited, Nottingham (GB); University of Nottigham, Nottingham (GB)

(21) Appl. No.: 11/993,335

(22) PCT Filed: Jun. 22, 2006
(86) PCT No.: PCT/GB06/02289

§ 371 Date: Jul. 7, 2010

(30) Foreign Application Priority Data
Jun. 22, 2005 (GB) 0512707.1

(51) Int. Cl. C12P 21/06; C12N 1/19; C12N 15/74

(52) U.S. Cl. 435/69.1; 435/254.21; 435/471

(57) ABSTRACT

The present invention provides a host cell suitable for enhanced production of a protein product of choice characterised in that the host cell is genetically modified to cause over-expression of two or more helper proteins selected from a DnaJ-like protein (such as JEM1), an Hsp70 family protein (such as LHS1) and SIL1 wherein at least one of the over-expressed two or more helper proteins is selected from JEM1, LHS1 and SIL1, and wherein the DnaJ-like protein is not SCJ1.

### FIELD OF THE INVENTION

The present application relates to gene expression techniques.

### BACKGROUND OF THE INVENTION

The listing or discussion of a prior-published document in this specification should not necessarily be taken as an acknowledgement that the document is part of the state of the art or is common general knowledge.

A key parameter in the development of a commercially viable process for the production of a recombinant protein is the yield of the product from the host organism.

Factors that influence the yield of a particular heterologous protein are complex and include the biochemical and biophysical properties of the protein itself; its influence on, and modification of, the host's own cellular functions; and the choice and deployment of those sequences that are necessary for efficient transcription, translation, secretion (if required) and plasmid stability.

### SUMMARY OF THE INVENTION

We have identified a series of proteins (hereinafter “helper” proteins) that are over-expressed in a (non-publicly available) S. cerevisiae that possesses increased production of a protein product of choice, such as a recombinant protein. These overexpressed helper proteins have all, individually, been previously identified.

In the case of some of these helper proteins, there is nothing in the art to suggest that their over-expression would aid in the increased production of a recombinant heterologous protein product of choice.

In the case of some of the other identified helper proteins, the (as yet unpublished) art has recognised that their over-expression can aid in increasing the production of a recombinant heterologous protein product of choice (see PCT/GB2004/005462). However, there is nothing in the art to suggest that the combined and simultaneous over-expression of such helper proteins would further enhance the production of a protein product of choice.

Accordingly, the present invention provides a host cell suitable for enhanced production of a protein product of choice wherein the host cell is genetically modified to cause over-expression of one or more of the identified helper proteins.

Thus the present invention provides a host cell that is suitable for enhanced production of a protein product of choice characterised in that the host cell comprises a first gene encoding a first helper protein as defined herein, or a variant thereof, and a second gene encoding a desired protein product of choice, wherein the host cell is genetically modified to cause over-expression of the first helper protein, and—
(a) wherein the first and second genes are not both present within the host cell on the same 2 μm-family plasmid (and optionally the first gene is not present within the host cell on any 2 μm-family plasmid; and further optionally the second gene is not present within the host cell on any 2 μm-family plasmid); and
(b) wherein the host cell is not genetically modified to cause over-expression of a further helper protein that is different from the first helper protein and is selected from the group consisting of AHA1, CCT2, CCT3, CCT4, CCT5, CCT6, CCT7, CCT8, CNS1, CPR3, CPR6, ERO1, EUG1, FMO1, HCH1, HSP10, HSP12, HSP104, HSP26, HSP30, HSP42, HSP60, HSP78, HSP82, JEM1, MDJ1, MDJ2, MPD1, MPD2, PDI1, PFD1, ABC1, APJ1, ATP11, ATP12, BTT1, CDC37, CPR7, HSC82, KAR2, LHS1, MGE1, MRS11, NOB1, ECM10, SSA1, SSA2, SSA3, SSA4, SSC1, SSE2, SIL1, SLS1, ORM1, ORM2, PER1, PTC2, PSE1, UBI4 and HAC1 or a truncated intronless HAC1 (and optionally, the host cell is not genetically modified to cause over-expression of any further helper protein that is different from the first helper protein).

The thus over-expressed first helper protein may be any helper protein defined below. For example, the over-expressed first helper protein may be a DnaJ-like protein (such as JEM1), an Hsp70 family member protein (such as LHS1) or SIL1, or a variant of any of these. Over-expression of the first helper protein may be achieved by any suitable means of genetic modification known in the art. Suitable examples of such approaches for genetic modification are discussed in more detail below.

The host cell may or may not comprise a recombinant copy, such as a plasmid encoded copy, or a chromosomally integrated recombinant copy, of a gene encoding the further helper protein as defined in (b) above. Thus, in one embodiment, the first helper protein may be the only helper protein that is over-expressed by the host cell.

In another embodiment, the invention provides a host cell that is suitable, for enhanced production of a protein product of choice characterised in that the host cell is genetically modified to cause over-expression of a helper protein selected from the list comprising SCJ1, FKB2, SSE1, ERV2, DER1, DER3, HRD3, UBC7 and DOA4. The host cell may or may not be genetically modified to cause over-expression of two or more helper proteins, at least one of which is a helper protein selected from the list comprising SCJ1, FKB2, SSE1, ERV2, DER1, DER3, HRD3, UBC7 and DOA4. In that case, at least one other helper may or may not be selected from the list comprising—
(a) chaperones selected from a DnaJ-like protein (such as JEM1), an Hsp70 family member protein (such as LHS1), SCJ1, KAR2, SIL1 (note that, SIL1 has previously been referred to as SLS1), FKB2, SSA1, SSA2, SSA3, SSA4, SSE1, SSE2, SSB1, SSB2, ECM10, MDJ1 and MDJ2.
(b) proteins involved in the formation of disulphide bonds in other proteins selected from ERO1, ERV2, EUG1, MPD1, MPD2, EPS1 and PDI1;
(c) proteins involved in protein degradation selected from DER1, DER3, HRD3, UBC7 and DOA4; and
(d) HAC1.

For example, the host cell may or may not be genetically modified to cause over-expression of two or more helper proteins selected from a DnaJ-like protein (such as JEM1), an Hsp70 family protein (such as LHS1) and SIL1. For example, the host cell according to may or may not be genetically modified to cause over-expression of—
(a) a DnaJ-like protein and an Hsp70 family protein; or
(b) a DnaJ-like protein and SIL1; or
(c) an Hsp70 family protein and SIL1.

The host may or may not be genetically modified to cause over-expression of three or more helper proteins, wherein the three or more helper proteins comprise a DnaJ-like protein, an Hsp70 family protein and SIL1 for example JEM1, LHS1 and SIL1.

The Hsp70 family protein may or may not be a protein that localises to the lumen of the ER. The Hsp70 family protein may or may not be a prokaryotic Hsp70 family protein. The Hsp70 family protein may or may not be a eukaryotic Hsp70 family protein. The Hsp70 family protein may or may not be LHS1, KAR2, SSA1, SSA2, SSA3, SSA4, SSE1, SSE2, SSB1, SSB2 or ECM10, such as from yeast, for example, from S. cerevisiae. LHS1 may or may not be a preferred Hsp70 family protein for use in the present invention. Other Hsp70 family proteins for use in the present invention may or may not include a mammalian BiP (GRP78) (, such as the protein described by Haas and Wabl (1983) Nature 306, 387), a mammalian HSP72 (HSP70), HSP73 (HSC70) or mtp70, a mammalian GRP170 (such as the protein described by Lin et al (1993) Mol. Biol. Cell 4, 1109), a mammalian HSP70 protein (such as a protein as reviewed by Ohtsuka and Hata. (2000) International Journal of Hyperthermia 16, 231; Gething and Sambrook (1992) Nature 355, 33; and/or Craig and Gross (1991) TIBS 16, 135), a Gallus gallus HSP70 protein, such as the protein defined by accession number to AAO44921 (Mazzi et al (2003) Genet. Mol. Biol. 26, 275-281), a Nicotiana tabacum luminal binding protein (BiP), such as the protein defined by accession number CAA42661 (Denecke et al (1991) Plant Cell 3, 1025), a Paramecium caudatum HSP70 protein, such as the protein defined by accession number BAE16705 (Hori et al (2006) Mol. Phylogenet. Evol. 38, 697), a Hordeum vulgare HSP70 protein, such as a subsp. vulgare HSP70 protein accession number, such as the protein defined by AAA62325 (Chen et al (1994) Plant Physiol. 106, 815), an Arabidopsis thaliana HSP70 protein accession number NP_{—}187864, the Chlamydia trachomatis A/HAR-13 chaperone protein dnaK (Heat shock protein 70) (Heat shock 70 kDa protein) (HSP70), such as the protein defined by accession number Q3KLV7 (Carlson et al (2005) Infect. Immun. 73, 6407), a Pongo pygmaeus hsp70 protein, such as the protein defined by accession number CAH92327, a Haemophilus influenzae 86-028NP HSP70 protein, such as the protein defined by accession number YP_{—}249343 (Harrison et al (2005) J. Bacteriol. 187, 4627), a Streptococcus pneumoniae HSP70 protein, such as the protein defined by accession number AAB39221, a Mus musculus HSP70 protein, such as the protein defined by accession number AAC84169 (Xie et al (2003) Genome Res. 13, 2621), a Bacillus subtilis HSP70 protein, such as the protein defined by accession number BAA12464 (Mizuno et al. (1996) Microbiology (Reading, Engl.) 142, 3103), and a Escherichia coli DnaK protein, such as the protein defined by Slepenkov and Witt (2002) Mol. Microbiol. 45, 1197. It will be appreciated that, in the rest of this specification, reference to LHS1 may or may not be taken to be, by extension, a reference to an equivalent Hsp70 family protein, such as an Hsp70 family protein as defined in this paragraph.

Other preferred Hsp70 family proteins may have an activity equivalent to LHS1, when co-expressed with one or both of JEM1 and SIL1 for example in the manner as set out in the present examples. Thus, a host cell of the present invention, when genetically modified to cause simultaneous over-expression of a preferred Hsp70 family protein with one or both of JEM1 and SIL1, will provide at least substantially the same increase in the production of a protein product and/or at least substantially the same reduction of fragmentation of a protein product, as is observed in the same host cell when genetically modified to cause simultaneous over-expression of LHS1 with one or both of JEM1 and SIL1 the increase being compared to the to the level of production of the same protein product, and/or the level of fragmentation of the same protein product, in the same host cell that has not been genetically modified to cause overexpression of any of LHS1, JEM1 or SIL1.

By “substantially the same increase in the production of a protein product”, we, mean at least 10%, 20%, 30%, 40%, 50%, 60%, 70%, 80%, 90%, 95%, 96%, 97%, 98%, 99%, substantially 100% or greater than 100% of the increase in production of a protein product that is observed when the host cell is genetically modified to cause simultaneous over-expression of LHS1 with one or both of JEM1 and SIL1 (the increased being compared to the level of production of the same protein product in the same host cell that has not been genetically modified to cause overexpression of any of LHS1, JEM1 or SIL1).

By “substantially the same reduction of fragmentation of a protein product”, we mean at least 10%, 20%, 30%, 40%, 50%, 60%, 70%, 80%, 90%, 95%, 96%, 97%, 98%, 99%, substantially 100% or greater than 100% of the reduction of fragmentation of a protein product that is observed when the host cell is genetically modified to cause simultaneous over-expression of LHS1 with one or both of JEM1 and SIL1 (the reduction of fragmentation of a protein product being compared to the level of fragmentation of the same protein product in the same host cell that has not been genetically modified to cause overexpression of any of LHS1, JEM1 or SIL1).

DnaJ-like proteins are reviewed in Walsh et al, 2004, EMBO reports, 5, 567-571. The DnaJ-like protein typically comprises a J-domain as defined in Walsh et al, 2004, op. cit. the contents of which are incorporated herein by reference. The DnaJ-like protein may or may not be a prokaryotic DnaJ-like protein. The DnaJ-like protein may or may not be a eukaryotic DnaJ-like protein. The DnaJ-like protein may or may not be any one of the yeast DnaJ proteins such as a protein selected from JEM1, MDJ1, MDJ2, SEC63, YDJ1, XDJ1, APJ1, SIS1, DJP1, ZUO1, SWA2, JJJ1, JJJ2, JJJ3, CAJ1, CWC23, PAM18, JAC1, JID1, SCJ1, HLJ1 to and ERJ5. The DnaJ-like protein may or may not be a protein that localises to the ER, such as JEM1, SCJ1, HLJ1, SEC63 or ERJ5, and may or may not be a protein that localises to the ER membrane. The DnaJ-like protein may or may not be a protein that localises to the cytoplasm of the host cell, such as YDJ1, XDJ1, APJ1, SIS1, DJP1, ZUO1, SWA2, JJJ1, JJJ2 or JJJ3. The DnaJ-like protein may or may not be a protein that localises to the nucleoplasm of the host cell, such as CAJ1 or CWC23. The DnaJ-like protein may or may not be a protein that localises to the mitochondria of the host cell, such as MDJ1, MDJ2, PAM18, JAC1 or JID1. The DnaJ-like protein is typically not SCJ1. JEM1 may or may not be a preferred DnaJ-like protein for use in the present invention. Other DnaJ-like proteins may or may not include the following proteins or proteins families, or fragments or variants thereof—
the HSP40 class of proteins (reviewed by Ohtsuka and Hata. (2000) International Journal of Hyperthermia 16, 231 and Table 1 therein);
a mammalian Erdj1 (such as MTJ1, Chevalier et al (2000) J. Biol. Chem. 275 19620);
a mammalian Erdj2 such as hSec63, Skowronek et al (1999) J. Biol. Chem. 380, 1133);
a mammalian Erdj3 (such as HEDJ/Scj1p, Shen and Hendershot (2005) Mol. Biol. Cell. 16, 40);
a mammalian Erdj4 (such as described in Shen et al (2002) J. Biol. Chem. 277, 15947);
a mammalian Erdj5 (such as described in Cunnea et al (2003) J. Biol. Chem. 278, 1059);
a Gallus gallus DnaJ homolog subfamily B member 11 precursor, such as the ER-associated dnaJ protein 3 ErJ3, the ER-associated Hsp40 co-chaperone (hDj9, or the PWP1-interacting protein 4, such as defined by accession number XP_{—}422682;
a Nicotiana tabacum DnaJ homolog, such as the protein defined by accession number BAC53943;
a Arabidopsis thaliana DnaJ homolog, such as the protein defined by accession number AAB49030 (Zhou et al (1999) Plant Physiol. 121, 1053);
a Chlamydia trachomatis A/HAR-13 Chaperone protein dnaJ, such as the protein defined by accession number YP_{—}328153 (Carlson et al (2005) Infect. Immun. 73, 6407);
a Pongo pygmaeus DnaJ homolog subfamily B member 9, such as the protein defined by accession number Q5R9A4;
a Haemophilus influenzae Rd KW20 Dna-J like membrane chaperone protein, such as the protein defined by accession number NP_{—}438440 (Fleischmann et al (1995) Science 269, 496);
a Escherichia coli DnaJ protein, such as the protein defined by accession number AAA00009 (Ohki et al (1986) J. Biol. Chem. 261, 1778);
a Escherichia coli DnaJ-like protein, such as the protein defined by accession number BAB96590 (Musso et al (1977) Proc. Natl. Acad. Sci. U.S.A. 74, 106);
a Streptococcus pneumoniae DnaJ protein, such as the protein defined by accession number AAB39222;
a Mus musculus DnaJ homolog, such as a subfamily B member 6 (Heat shock protein. J2) (HSJ-2) (MRJ) (mDj4) such as the protein defined by accession number XP_{—}987742;
a Bacillus subtilis DnaJ protein, such as the protein defined by accession number BAA12465 (Mizuno et al (1996) Microbiology (Reading Engl.) 142, 3103); and
a plant Sorghum bicolour DNAJ domain protein, such as the protein defined by accession number ABF48023.

It will be appreciated that, in the rest of this specification, reference to JEM1 may or may not be taken to be, by extension, a reference to an equivalent DnaJ-like protein, such as a DnaJ-like protein as defined in the above paragraph.

Other preferred DnaJ-like proteins may have an activity equivalent to JEM1, when co-expressed with one or both of LHS1 and SIL1, for example in the manner as set out in the present examples. Thus, a host cell of the present invention, when genetically modified to cause simultaneous over-expression of a preferred DnaJ-like protein with one or both of LHS1 and SIL1, will provide at least substantially the same increase in the production of a protein product and/or at least substantially the same reduction of fragmentation of a protein product, as is observed in the same host cell when genetically modified to cause simultaneous over-expression of JEM1 with one or both of LHS1 and SIL1, the increase being compared to the level of production of the same protein product, and/or the level of fragmentation of the same protein product, in the same host cell that has not been genetically modified to cause overexpression of any of LHS1, JEM1 or SIL1.

By “substantially the same increase in the production of a protein product”, we mean at least 10%, 20%, 30%, 40%, 50%, 60%, 70%, 80%, 90%, 95%, 96%, 97%, 98%, 99%, substantially 100% or greater than 100% of the increase in production of a protein product that is observed when the host cell is genetically modified to cause simultaneous over-expression of JEM1 with one or both of LHS1 and SIL1 (the increase being compared to the level of production of the same protein product in the same host cell that has not been genetically modified to cause overexpression of any of LHS1, JEM1 or SIL1).

By “substantially the same reduction of fragmentation of a protein product”, we mean at least 10%, 20%, 30%, 40%, 50%, 60%, 70%, 80%, 90%, 95%, 96%, 97%, 98%, 99%, substantially 100% or greater than 100% of the reduction of fragmentation of a protein product that is observed when the host cell is genetically modified to cause simultaneous over-expression of JEM1 with one or both of LHS1 and SIL1 (the reduction of fragmentation of a protein product being compared to the level of fragmentation of the same protein product in the same host cell that has not been genetically modified to cause overexpression of any of LHS1, JEM1 or SIL1).

The host cell that is genetically modified to cause over-expression of two or more; such as at least three, helper proteins selected from a DnaJ-like protein, an Hsp70 family protein and SIL1 may or may not be further genetically modified to cause over-expression of at least one, two, three, four, five, six or seven proteins involved in the formation of disulphide bonds in other proteins selected from the group consisting of ERO1, ERV2, EUG1, MPD1, MPD2, EPS1 and PDI1. PDI1 may or may not be preferred.

In another embodiment, the invention provides a host cell suitable for enhanced production of a protein product of choice characterised in that the host cell is genetically modified to cause over-expression of three or more helper proteins, wherein the three or more helper proteins are selected from the list comprising—
(a) chaperones selected from a DnaJ-like protein (such as JEM1), an Hsp70 family member protein (such as LHS1), SCJ1, KAR2, SIL1, FKB2, SSA1, SSA2, SSA3, SSA4, SSE1, SSE2, SSB1, SSB2, ECM10, MDJ1 and MDJ2.
(b) proteins involved in the formation of disulphide bonds in other proteins selected from ERO1, ERV2, EUG1, MPD1., MPD2, EPS1 and PDI1;
(c) proteins involved in protein degradation selected from DER1, DER3, HRD3, UBC7 and DOA4; and
(d) HAC1.

The three or more helper proteins may or may not comprise at least one, two, three, four, five, six, seven, eight, nine, ten, eleven, twelve, thirteen, fourteen, fifteen, sixteen or seventeen of the chaperones selected from the group consisting of JEM1, an Hsp70 family member protein (such as LHS1), SCJ1, KAR2, SIL1, FKB2, SSA1, SSA2, SSA3, SSA4, SSE1, SSE2, SSB1, SSB2, ECM10, MDJ1 and MDJ2. The three or more helper proteins may or may not comprise at least one, two, three, four, five, six or seven proteins involved in the formation of disulphide bonds in other proteins selected from the group consisting of ERO1, ERV2, EUG1, MPD1, MPD2, EPS1 and PDI1. The three or more helper proteins may or may not comprise at least one, two, three, four or five of the proteins involved in protein degradation selected from DER1, DER3, HRD3, UBC7 and DOA4.

It will be appreciated that the host cell may or may not comprise a polynucleotide sequence that encodes a protein product of choice.

In one embodiment, the host cell comprises a polynucleotide sequence that encodes a protein product of choice. The protein product of choice may or may not be a protein that is naturally produced by the host cell or may or may not be a heterologous protein. In this context, a “heterologous protein” is a protein that is not naturally encoded by the host cell. The polynucleotide sequence that encodes the protein product of choice may or may not be an endogenous polynucleotide sequence or (in particular, where the protein product of choice is a heterologous protein) the polynucleotide sequence that encodes the protein product of choice may or may not be an exogenous polynucleotide, and the exogenous polynucleotide may or may not be integrated into the chromosome of the host cell or present in the host cell as part of a replicable vector, such as a plasmid.

However, the present invention also contemplates the production of host cells suitable for enhanced production of a protein product of choice, into which an appropriate polynucleotide sequence, encoding the protein product of choice, can be later introduced. Therefore, in another embodiment, the host cell does not comprise a polynucleotide sequence that encodes a protein product of choice.

Suitable host cells are discussed below.

By “enhanced production” we include the meaning that the level of production of protein product of choice is greater in a cultured population of the genetically modified host cell than in a cultured population of the same host cell that has not been genetically modified to cause over-expression of one or more of the identified helper proteins. Typically, the measurement can be made under culture conditions that are standard for the growth of the host cell that is being used.

Thus the production of the protein product of choice in a cultured population of the genetically modified host cell of the invention be greater than, typically at least 1%, 2%, 3%, 4%, 5%, 6%, 7%, 8%, 9%, 10% (i.e. 1.1-fold), 20% (i.e. 1.2-fold), 30% (i.e. 1.3-fold), 40% (i.e. 1.4-fold), 50% (i.e. 1.5-fold), 60% (i.e. 1.6-fold), 70% (i.e. 1.7 fold), 80% (i.e. 1.8-fold), 90% (i.e. 1.9-fold), 100% (i.e. 2-fold), 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, or 1000-fold greater than, the production of the protein product of choice in a cultured population of the same host cell that has not been genetically modified to cause over-expression of one or more of the identified helper proteins. These figures may, or may not, be figures that have been normalised to account for differences in the cell growth of the two cultured populations, as compared.

For example, the production of the protein product of choice in a cultured population of the genetically modified host cell of the invention may be up to 10% (i.e. 1.1-fold), 20% (i.e. 1.2-fold), 30% (i.e. 1.3-fold), 40% (i.e. 1.4-fold), 50% (i.e. 1.5-fold), 60% (i.e. 1.6-fold), 70% (i.e. 1.7 fold), 80% (i.e. 1.8-fold), 90% (i.e. 1.9-fold), 100% (i.e. 2-fold), 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold or 2000-fold greater than the production of the protein product of choice in a cultured population of the same host cell that has not been genetically modified to cause over-expression of one or more of the identified helper proteins. These figures may, or may not, be figures that have been normalised to account for differences in the cell growth of the two cultured populations, as compared.

Typically, the protein product of choice may be produced in a cultured population of the genetically modified host cell of the invention to produce a culture containing at least 0.001 g·L⁻¹, such as at least 0.01 at least 0.1 g·L⁻¹, 1 g·L⁻¹, 2 g·L⁻¹, 3 g·L⁻¹, 4 g·L⁻¹, 5 g·L⁻¹, 6 g·L⁻¹, 7 g·L⁻¹, 8 g·L⁻¹, 9 g·L⁻¹, 10 g·L⁻¹, 20 g·L⁻¹, 30 g·L⁻¹, 40 g·L⁻¹, 50 g·L⁻¹, 60 g·L⁻¹, 70 g·L⁻¹, 80 g·L⁻¹, 90 g·L⁻¹, or 100 g·L⁻¹ of the protein product of choice. The protein product of choice may be produced in a cultured population of the genetically modified host cell of the invention to produce a culture containing up to 0.01 g·L⁻¹, 0.1 g·L⁻¹, 2 g·L⁻¹, 3 g·L⁻¹, 4 g·L⁻¹, 5 g·L⁻¹, 6 g·L⁻¹, 7 g·L⁻¹, 8 g·L⁻¹, 9 g·L⁻¹, 10 g·L⁻¹, 20 g·L⁻¹, 30 g·L⁻¹, 40 g·L⁻¹, 50 g·L⁻¹, 60 g·L⁻¹, 70 g·L⁻¹, 80 g·L⁻¹, 90 g·L⁻¹, 100 g·L⁻¹ or 200 g·L⁻¹ of the protein product of choice.

By “enhanced production” we also include the meaning that the level of activity of the protein product of choice that is produced by the host cell is greater in a cultured population of the genetically modified host cell than in a cultured population of the same host cell that has not been genetically modified to cause over-expression of one or more of the identified helper proteins. The nature of the activity will depend on the identity of the protein product of choice and may, for example, be a measurement of the catalytic activity of the protein upon a substrate or the binding properties of the protein to a ligand. Typically, the measurement of protein activity can be made under culture conditions that are standard for the growth of the host cell that is being used or following isolation of the protein from the culture medium. In either case, the comparison should be made on the basis of activity per unit volume of culture or protein recovered therefrom. The comparison may, or may not, be normalised to account for differences in the cell growth of the two cultured populations, as compared.

Thus the activity of the protein product of choice that is produced in a cultured population of the genetically modified host cell of the invention may be greater than, typically at least 10% (i.e. 1.1-fold), 20% (i.e. 1.2-fold), 30% (i.e. 1.3-fold), 40% (i.e. 1.4-fold), 50% (i.e. 1.5-fold), 60% (i.e. 1.6-fold), 70% (i.e. 1.7-fold), 80% (i.e. 1.8-fold), 90% (i.e. 1.9-fold), 100% (i.e. 2-fold), 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 10⁴-fold, 10⁵-fold, or 10⁶-fold greater than, the activity of the protein product of choice in a cultured population of the same host cell that has not been genetically modified to cause over-expression of one or more of the identified helper proteins.

For example, the activity of the protein product of choice in a cultured population of the genetically modified host cell of the invention may be up to 10% (i.e. 1.1-fold), 20% (i.e. 1.2-fold), 30% (i.e. 1.3-fold), 40% (i.e. 1.4-fold), 50% (i.e. 1.5-fold), 60% (i.e. 1.6-fold), 70% (i.e. 1.7 fold), 80% (i.e. 1.8-fold), 90% (i.e. 1.9-fold), 100% (i.e. 2-fold), 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 10⁴-fold, 10⁵-fold, or 10⁶-fold greater than the activity of the protein product of choice in a cultured population of the same host cell that has not been genetically modified to cause over-expression of one or more of the identified helper proteins.

By “enhanced production” we include the additional or alternative meaning that the level of degradation of the protein product of choice is reduced when produced by a cultured population of the genetically modified host cell of the present invention compared to the level of degradation of the protein product of choice when produced by a cultured population of the same host cell that has not been genetically modified to cause over-expression of one or more of the identified helper proteins according to the present invention. The level of protein degradation can be determined by quantification of fragments of the protein product of choice relative to the total of the protein product of choice, for example when by analysis of SDS-PAGE using densitometry. When expressed as a percentage of detected protein product fragments relative to total protein product levels detected (i.e. total protein product detected=full length protein product+degradation products) then the percentage of detected protein product fragments when produced by a cultured population of the genetically modified host cell of the present invention may be, or be less than, 99%, 98%, 97%, 96%, 05%, 04%, 03%, 92%, 90%, 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, 1% or less, such as up to 98%, 97%, 96%, 95%, 94%, 93%, 92%, 90%, 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, 1% or less of the percentage of detected protein product fragments when produced by a cultured population of the same host cell that has not been genetically modified to cause over-expression of one or more of the identified helper proteins according to the present invention. These values may or may not be normalised, for example based on culture optical density readings, to account for different growth rates observed between strains.

By “enhanced production” we include the additional or alternative meaning that the level of post-translational modification of the protein product of choice is increased or reduced when produced by a cultured population of the genetically modified host cell of the present invention compared to the level of post-translational modification of the protein product of choice when produced by a cultured population of the same host cell that has not been genetically modified to cause over-expression of one or more of the identified helper proteins according to the present invention. For example, the altered (i.e. increased or reduced) level of post-translational modification may be an alteration in the level of proteolytic cleavage, hexosylation (for example mannosylation), glycosylation, phosphorylation, phosphopantetheinylation, carbamylation, carboxylation (such as γ-carboxylation), sialation, sulphonation, hydroxylation, prenylation, isoprenylation, acylation, ubiquitination, lipoylation, biotinylation, glycylation, glutamylation, methylation, alkylation, acetylation, formylation, selenation, disulphide bond formation or oligomerisation of the protein product of choice. The level of post-translational modification of the protein product of choice can be determined by methods well known in the art, such as by mass spectrometry techniques (for example, see Larsen et al, 2006, BioTechniques, 40, 790-798) well known in the art.

By “enhanced production” we include the additional or alternative meaning that the level of stress experienced by a cell that is being cultured to produce the protein product of choice is reduced, compared to the level of stress experienced by a cultured population of the same host cell that has not been genetically modified to cause over-expression of one or more of the identified helper proteins according to the present invention. For example, “enhanced production” can include the additional or alternative meaning that the unfolded protein response is reduced in a host cell. The level of stress, and the level of the unfolded protein response, can be measured by determination of the proportion of HAC1ⁱ to total HAC1 transcript levels. Total HAC1 transcript levels are the sum of HAC1ⁱ transcript levels and unspliced HAC1 (HAC1^{u}) transcript levels in a cell. A reduced proportion of HAC1ⁱ transcript levels compared to total HAC1 transcript level, relative to a control, is indicative of reduced stress and reduced. UPR signalling relative to that control. Helper proteins suitable for achieving this effect may include Hsp70 family proteins (such as LHS1) and DnaJ-like proteins (such as JEM1) and combinations of other helper proteins such as disclosed in the present application.

In principle, any “protein product of choice” can be produced. The identity of preferred embodiments of the “protein product of choice” is discussed further below.

The host cell is genetically modified to cause over-expression of one or more of the helper proteins. By “over-expression”, in the context of helper proteins, we mean that the measurable level of mRNA encoding the one or more helper proteins, and/or the measurable level of the one or more helper proteins themselves, and/or the measurable level of the helper protein activity, is greater than the measurable level in a host cell that has not been genetically modified. Typically, the measurement will be made under culture conditions that are standard for the growth of the host cell that is being used. Standard conditions for yeast cell growth are discussed, for example, in WO 96/37515, WO 00/44772 and WO 99/00504, the contents of which are incorporated herein by reference.

Thus the host cell may or may not be genetically modified to cause a level of expression of one or more of the helper proteins that is at least a 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold or more, of unmodified level of expression of one or more of the helper proteins.

For example, the host cell may or may not be genetically modified to cause a level of expression of one or more of the helper proteins that is up to 1.2-fold, 1.3-fold, 1.4-fold, 1.5-fold, 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold or 100-fold of the unmodified-type level of expression of one or more of the helper proteins.

For example, the host cell may be genetically modified to cause a level of expression of one or more of the helper proteins that is between 1- to 30-fold, such as about 2- to 25-fold, of the unmodified-type level of expression of one or more of the helper proteins.

The host cell may or may not be genetically modified to cause over-expression of one or more of the helper proteins by the introduction of one or more recombinant copies of one or more polynucleotides that each comprise a region (the “coding region”, or “open reading frame”, which can be abbreviated to “ORF”) that encodes one or more helper proteins.

A copy of the polynucleotide may or may not be introduced into the chromosome of the host cell and/or may or may not be encoded by a plasmid or other vector that is used to transform the host cell.

The polynucleotide may or may not comprise some or all of the regulatory sequences necessary to cause transcription and/or translation of the ORF of the polynucleotide.

Regulatory sequences necessary to cause transcription and/or translation of the ORF of the polynucleotide include sequences that modulate (i.e., promotes or reduces, typically promotes) the expression (i.e., the transcription and/or translation) of an ORF to which it is operably linked. Regulatory regions typically include promoters, terminators, ribosome binding sites and the like. The skilled person will appreciate that the choice of regulatory region will depend upon the intended expression system. For example, promoters may or may not be constitutive or inducible and may or may not be cell- or tissue-type specific or non-specific.

Suitable regulatory regions, may be about, or up to, 5 bp, 10 bp, 15 bp, 20 bp, 25 bp, 30 bp, 35 bp, 40 bp, 45 bp, 50 bp, 60 bp, 70 bp, 80 bp, 90 bp, 100 bp, 120 bp, 140 bp, 160 bp, 180 bp, 200 bp, 220 bp, 240 bp, 260 bp, 280 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1000 bp, 1100 bp, 1200 bp, 1300 bp, 1400 bp, 1500 bp or greater, in length.

Such non-coding regions and regulatory regions are not restricted to the native non-coding regions and/or regulatory regions naturally associated with the ORF.

Where the host cell is yeast, such as Saccharomyces cerevisiae, suitable promoters for S. cerevisiae include those associated with the PGK1 gene, GAL1 or GAL10 genes, TEF1, TEF2, PYK1, PMA1, CYC1, PHO5, TRP1, ADH1; ADH2, the genes for glyceraldehyde-3-phosphate dehydrogenase (for example, TDH1, TDH2 or TDH3), hexokinase (for example, HXK1 or HXK2), pyruvate decarboxylase (for example, PDC1, PDC5 or PDC6), phosphofructokinase (for example, PFK1 or PFK2), triose phosphate isomerase (for example, TPI1), phosphoglucose isomerase (for example, PGI1), glucokinase (for example, GLK1), α-mating factor pheromone (for example, MFα-1 or MFα-2), α-mating factor pheromone (for example, MFA1 or MFA2), PRB1, PRA1, GPD1, and hybrid promoters involving hybrids of parts of 5′ regulatory regions with parts of 5′ regulatory regions of other promoters or with upstream activation sites (e.g. the promoter of EP-A-258 067). Where multiple ORFs are to be expressed, a different promoter may or may not be chosen for each ORF. The skilled person can readily determine appropriate combinations of promoters. For example, the promoters from the ADH1, PGK1, TDH1 and TEF1 genes are used in combination to recombinantly over-express four helper proteins in Example 3 below.

Suitable transcription termination signals are well known in the art. Where the host cell is eukaryotic, the transcription termination signal is preferably derived from the 3′ flanking sequence of a eukaryotic gene, which contains proper signals for transcription termination and polyadenylation. Suitable 3′ flanking sequences may, for example, be those of the gene naturally linked to the expression control sequence used, i.e. may correspond to the promoter. Alternatively, they may be different. In that case, and where the host is a yeast, preferably S. cerevisiae, then the termination signal of the S. cerevisiae ADH1, ADH2, CYC1, or PGK1 genes are preferred.

It may be beneficial for the promoter and open reading frame to be flanked by transcription termination sequences so that the transcription termination sequences are located both upstream and downstream of the promoter and open reading frame, in order to prevent transcriptional read-through into neighbouring genes, and visa versa.

In one embodiment, a suitable regulatory sequences in yeast, such as Saccharomyces cerevisiae, includes: a yeast promoter (e.g. the Saccharomyces cerevisiae PRB1 promoter), as taught in EP 431 880; and a transcription terminator, preferably the terminator from Saccharomyces ADH1, as taught in EP 60 057. Other suitable regulatory sequences are given in the examples, and include TEF1, PGK1 and TDH1 promoters.

It may be beneficial for the non-coding region to incorporate more than one DNA sequence encoding a translational stop codon, such as UAA, UAG or UGA, in order to minimise translational read-through and thus avoid the production of elongated, non-natural fusion proteins. The translation stop codon UAA is preferred. Preferably, the polynucleotide incorporates at least two translation stop codons.

The term “operably linked” includes within its meaning that a regulatory sequence is positioned within any non-coding region in a gene such that it forms a relationship with an ORF that permits the regulatory region to exert an effect on the ORF in its intended manner. Thus a regulatory region “operably linked” to an ORF is positioned in such a way that the regulatory region is able to influence transcription and/or translation of the ORF in the intended manner, under conditions compatible with the regulatory sequence.

Alternatively, the polynucleotide may or may not be formed in such a manner that it can take advantage of endogenous regulatory sequences within the chromosome or plasmid to cause transcription and/or translation of the coding region of the polynucleotide. For example, the use of promoterless constructs is well known in the art as a way of allowing an endogenous promoter sequence to drive the expression of a recombinantly-introduced polynucleotide coding region.

The skilled person will appreciate that the host cell may or may not comprise endogenous copies of genes encoding one or more of the helper proteins. Therefore, this invention also contemplates genetic modifications to the host cell that cause increased steady state levels of mRNA molecules encoding one or more helper proteins and/or increased steady state levels of one or more helper proteins.

This can include the genetic modification of operably linked endogenous regulatory regions. For example, the endogenous promoter in the gene of an endogenously encoded helper protein can be replaced by a promoter that causes greater levels of expression of the helper protein under culture conditions.

Alternatively, genetic modifications can be made to cis or trans regulators of the gene of an endogenously encoded helper protein, so as to increase the expression of the helper protein under culture conditions. Thus, the polynucleotide region that encodes a genetically encoded repressor of a gene of an endogenously encoded helper protein could be genetically modified to reduce or prevent repression of the endogenous helper protein gene.

Alternative genetic modifications to increase the expression of a helper protein or protein product of choice can involve transient expression techniques known in to the art. For example, suitable techniques are disclosed in Chen et al, 1997, Nucleic Acids Research, 25, 4416-4418 and in Behr et al, 1989, Proc. Natl. Acad. Sci. USA, 86, 6982-6986.

Thus, a number of techniques are available to the skilled person to genetically modify a cell to cause over-expression of a helper protein (and the same techniques may be used to cause expression of a protein product of choice). Suitable techniques include—
(i) introduction of a recombinant copy of an encoding polynucleotide by integration into the chromosome of the host cell (for example, either with associated regulatory sequences or without associated regulatory sequences so as to take advantage of endogenous regulatory sequences at the site of integration);
(ii) introduction of plasmid or other vector comprising a recombinant copy of an encoding polynucleotide into the cell;
(iii) genetic modifications of a host cell's endogenous regulatory region operably linked to the host cell's endogenous copy of an ORF encoding a helper protein or protein product of choice, to cause increased steady state levels of mRNA molecules encoded by said ORF;
(iv) genetic modifications to a cis or trans regulator of the gene of an endogenously encoded helper protein or protein product of choice; or
(v) transient expression of a helper protein or protein product of choice.

Where the host cell comprises a first gene encoding a protein product of choice, and a second gene encoding a first helper protein, then for example,
the first gene may be a gene as defined in (i) above and the second gene may be a gene as defined in (i), (ii), (iii), (iv) or (v) above;
the first gene may be a gene as defined in (ii) above and the second gene may be a gene as defined in (i), (ii), (iv) or (v) above (and where both the first and second genes are introduced on plasmid or vector, the first gene may or may not be introduced on the same plasmid or vector as the to second gene);
the first gene may be a gene as defined in (iii) above and the second gene may be a gene as defined in (i), (iv) or (v) above;
the first gene may be a gene as defined in (iv) above and the second gene may be a gene as defined in (i), (iv) or (v) above; or
the first gene may be a gene as defined in (v) above and the second gene may be a gene as defined in (i), (iv) or (v) above.

Where the host cell comprises a first gene encoding a protein product of choice, and a second gene encoding a first helper protein and a third gene encoding a second helper protein, then for example,
the first gene may be a gene as defined in (i) above, and the second gene may be a gene as defined in (i) above, and the third gene may be a gene as defined in (i), (iv) or (v) above;
the first gene may be a gene as defined in (i) above, and the second gene may be a gene as defined in (ii) above, and the third gene may be a gene as defined in (i), (ii), (iv) or (v) above (and where both the second and third genes are introduced on plasmid or vector, the second gene may or may not be introduced on the same plasmid or vector as the third gene);
the first gene may be a gene as defined in (i) above, and the second gene may be a gene as defined in (iii) above, and the third gene may be a gene as defined in (i), (ii), (iv) or (v) above;
the first gene may be a gene as defined in (i) above, and the second gene may be a gene as defined in (iv) above, and the third gene may be a gene as defined in (i), (ii), (iii), (iv) or (v) above;
the first gene may be a gene as defined in (i) above, and the second gene may be a gene as defined in (v) above, and the third gene may be a gene as defined in (i), (ii), (iii), (iv) or (v) above;
the first gene may be a gene as defined in (ii) above, and the second gene may be a gene as defined in (i) above, and the third gene may be a gene as defined in (i), (iii), (iv) or (v) above (and where both the first and third genes are introduced on plasmid or vector, the first gene may or may not be introduced on the same plasmid or vector as the third gene);
the first gene may be a gene as defined in (ii) above, and the second gene may be a gene as defined in (ii) above, and the third gene may be a gene as defined in (i), (iv) or (v) above (and where the first, second and third genes are introduced on plasmid or vector, the first gene may or may not be introduced on the same plasmid or vector as the second gene, the first gene may or may not be introduced on the same plasmid or vector as the third gene and the second gene may or may not be introduced on the same plasmid or vector as the third gene);
the first gene may be a gene as defined in (ii) above, and the second gene may be a gene as defined in (iii) above, and the third gene may be a gene as defined in (i), (iii), (iv) or (v) above;
the first gene may be a gene as defined in (ii) above, and the second gene may be a gene as defined in (iv) above, and the third gene may be a gene as defined in (i), (iv) or (v) above;
the first gene may be a gene as defined in (ii) above, and the second gene may be a gene as defined in (V) above, and the third gene may be a gene as defined in (i), (iv) or (v) above;
the first gene may be a gene as defined in (iii) above, and the second gene may be a gene as defined in (i) above,- and the third gene may be a gene as defined in (i), (iii), (iv) or (v) above;
the first gene may be a gene as defined in (iii) above, and the second gene may be a gene as defined in (ii) above, and the third gene may be a gene as defined in (i), (iv) or (v) above (and where both the second and third genes are introduced on plasmid or vector, the second gene may or may not be introduced on the same plasmid or vector as the third gene);
the first gene may be a gene as defined in (i) above, and the second gene may be a gene as defined in (iii) above, and the third gene may be a gene as defined in (i), (iv) or (v) above;
the first gene may be a gene as defined in (iii) above, and the second gene may be a gene as defined in (iv) above, and the third gene may be a gene as defined in (i), (iv) or (v) above;
the first gene may be a gene as defined in (iii) above, and the second gene may be a gene as defined in (v) above, and the third gene may be a gene as defined in (i), (iv) or (v) above;
the first gene may be a gene as defined in (iv) above, and the second gene may be a gene as defined in (i) above, and the third gene may be a gene as defined in (i), (ii), (iv) or (v) above;
the first gene may be a gene as defined in (iv) above, and the second gene may be a gene as defined in (ii) above, and the third gene may be a gene as defined in (i), (iii), (iv) or (v) above (and where both the second and third genes are introduced on plasmid or vector, the second gene may or may not be introduced on the same plasmid or vector as the third gene);
the first gene may be a gene as defined in (iv) above, and the second gene may be a gene as defined in (iii) above, and the third gene may be a gene as defined in (i), (iv) or (v) above;
the first gene may be a gene as defined in (iv).above, and the second gene may be a gene as defined in (iv) above, and the third gene may be a gene as defined in (i), (iv) or (v) above;
the first gene may be a gene as defined in (iv) above, and the second gene may be a gene as defined in (v) above, and the third gene may be a gene as defined in (i), (iv) or (v) above;
the first gene may be a gene as defined in (v) above, and the second gene may be a gene as defined in (i) above, and the third gene may be a gene as defined in (i), (ii), (iv) or (v) above;
the first gene may be a gene as defined in (v) above, and the second gene may be a gene as defined in (ii) above, and the third gene may be a gene as defined in (i), (iv) or (v) above (and where both the second and third genes are introduced on plasmid or vector, the second gene may or may not be introduced on the same plasmid or vector as the third gene);
the first gene may be a gene as defined in (v) above, and the second gene may be a gene as defined in (iii) above, and the third gene may be a gene as defined in (i), (iv) or (v) above;
the first gene may be a gene as defined in (v) above, and the second gene may be a gene as defined in (iv) above, and the third gene may be a gene as defined in (i), (iii), (iv) or (v) above; or
the first gene may be a gene as defined in (v) above, and the second gene may be a gene as defined in (v) above, and the third gene may be a gene as defined in (i), (iv) or (v) above.

Further combinations of possible genetic modifications will be apparent to the skilled person, in light of the above disclosure, when further genes (for example a fourth gene encoding a third helper protein; a fifth gene encoding a fourth helper protein, etc.) are to be over-expressed in the host cell of the invention.

The skilled person can readily choose the most appropriate and convenient method to achieve over-expression of one or more helper proteins in a host cell. It will be appreciated that, in the case that multiple helper proteins are over-expressed in the host cell, at least one helper protein may or may not be over-expressed by the introduction of an appropriate recombinant polynucleotide sequence as discussed above, whereas at least one other helper protein may or may not be over-expressed by a genetic modification to the host cell to cause over-expression of the helper protein from the endogenous gene that encodes it.

### Helper Proteins

As discussed above, we have identified a series of proteins (hereinafter “helper” proteins) that are over-expressed in a S. cerevisiae strain identified as possessing increased production of a recombinant protein. These over-expressed helper proteins have all, individually, been previously identified.

The helper proteins identified include proteins that can be categorised as follows—

(i) chaperones,
(ii) proteins involved in disulphide bond formation,
(iii) proteins involved in the protein degradation pathway, and
(iv) HAC1 (encoded by a spliced or unspliced polynucleotide).

These groups are individually described further below.

### Chaperones

The class of proteins known as chaperones have been defined by Hartl (1996, Nature, 381, 571-580) as a protein that binds to and stabilises an otherwise unstable conformer of another protein and, by controlled binding and release, facilitates its correct fate in vivo, be it folding, oligomeric assembly, transport to a particular subcellular compartment, or disposal by degradation.

For the purposes of the present invention, chaperones of interest can be broadly split into the following three functional sub-groups—
ER luminal localised chaperones;
Chaperones involved in cytoplasmic folding and maintenance of proteins in a translocation competent state prior to translocation; and
Mitochondrial chaperone and translocation proteins

Each of these groups are discussed in more detail below.

### ER Luminal Localised Chaperones

ER luminal localised chaperones, involved in “protein folding” include DnaJ-like proteins (such as JEM1), Hsp70 family member proteins (such as LHS1), SCJ1, KAR2, SIL1 and FKB2. A detailed description of these proteins and their genes is given separately below.

In one embodiment, the host cell may or may not be genetically modified to cause over-expression of one, or more, of the above ER luminal localised chaperones. For example, SCJ1 may or may not be over-expressed. Alternatively, FKB2 may or may not be over-expressed.

In another embodiment, the host cell may or may not be genetically modified to cause over-expression of two of the above ER luminal localised chaperones. For example, one of the following combinations may or may not be chosen—
A DnaJ-like proteins (such as JEM1) in combination with one of an Hsp70 family member protein (such as LHS1), SCJ1, KAR2, SIL1 or FKB2;
An Hsp70 family member protein (such as LHS1) in combination with one of SCJ1, KAR2, SIL1 or FKB2;
SCJ1 in combination with one of KAR2, KU or FKB2;
KAR2 in combination with one of SIL1 or FKB2; or
SIL1 in combination with FKB2.

In another embodiment, the host cell may or may not be genetically modified to cause over-expression of three of the above ER luminal localised chaperones. For example, one of the following combinations may or may not be chosen—

JEM1, LHS1 and SCJ1; JEM1, LHS1 and KAR2; JEM1, LHS1 and SIL1; JEM1, LHS1 and FKB2; JEM1, SCJ1 and KAR2; JEM1, SCJ1 and SIL1; JEM1, SCJ1 and FKB2; JEM1, KAR2 and SIL1; JEM1, KAR2 and FKB2; JEM1, SIL1 and FKB2; LHS1, SCJ1 and KAR2; LHS1, SCJ1 and SIL1; LHS1, SCJ1 and FKB2; LHS1, KAR2 and SIL1 LHS1, KAR2 and FKB2; LHS1, SIL1 and FKB2; SCJ1, KAR2 and SIL1; SCJ1, KAR2 and FKB2; SCJ1, SIL1 and FKB2; or KAR2, SIL1 and FKB2.

In one embodiment, the host cell may or may not be genetically modified to cause over-expression of four of the above ER luminal localised chaperones. For example, one of the following combinations may or may not be chosen—

JEM1, LHS1, SCJ1 and KAR2; JEM1, LHS1, SCJ1 and SIL1; JEM1, LHS1, SCJ1 and FKB2; JEM1, LHS1, KAR2 and SIL1; JEM1, LHS1, KAR2 and FKB2; JEM1, LHS1, SIL1 and FKB2; JEM1, SCJ1, KAR2 and SIL1; JEM1, SCJ1, KAR2 and FKB2; JEM1, SCJ1, SIL1 and FKB2; JEM1, KAR2, SIL1 and FKB2; LHS1, SCJ1, KAR2 and SIL1; LHS1, SCJ1, KAR2 and FKB2; LHS1, SCJ1, SIL1 and FKB2; LHS1, KAR2, SIL1 and FKB2; or SCJ1, KAR2, SIL1 and FKB2.

In another embodiment, the host cell may or may not be genetically modified to cause over-expression of five of the above ER luminal localised chaperones. For example, one of the following combinations may or may not be chosen—

JEM1, LHS1, SCJ1, KAR2 and SIL1; JEM1, LHS1, SCJ1, KAR2 and FKB2; JEM1, LHS1, SCJ1, SIL1 and FKB2; JEM1, LHS1, KAR2, SIL1 and FKB2; JEM1, SCJ1, KAR2, SIL1 and FKB2; or LHS1, SCJ1, KAR2, SIL1 and FKB2.

In another embodiment, the host cell may or may not be genetically modified to cause over-expression of all six of the above ER luminal localised chaperones. In other words, the following combination may or may not be chosen—

### JEM1, LHS1, SCJ1, KAR2, SIL1 and FKB2.

In one preferred embodiment, the host cell may or may not be genetically modified to cause over-expression of two, three or four helper proteins selected from LHS1, SELL JEM1 and SCJ1, such as one of the following combinations—

### LHS1 and SIL1 LHS1 and JEM1; LHS1 and SCJ1; SIL1 and JEM1; SIL1 and SCJ1; JEM1 and SCJ1; LHS1, SIL1 and JEM1; LHS1, SIL1 and SCJ1; LHS1, JEM1 and SCJ1; SIL1 JEM1 and SCJ1; or LHS1, SIL1 JEM1 and SCJ1.

### Chaperones Involved in Cytoplasmic Folding and Maintenance of Proteins in a Translocation Competent State Prior to Translocation

Chaperones involved in cytoplasmic folding and maintenance of proteins in a translocation competent state prior to translocation include SSA1, SSA2, SSA3, SSA4, SSE1, SSE2, SSB1, SSB2. A detailed description of these proteins and their genes is given separately below.

In one embodiment, the host cell may or may not be genetically modified to cause over-expression of one of the above chaperones involved in cytoplasmic folding and maintenance of proteins in a translocation competent state prior to translocation. For example, SSE1 may or may not be chosen.

In another embodiment, the host cell may or may not be genetically modified to cause over-expression of two of the above chaperones involved in cytoplasmic folding and maintenance of proteins in a translocation competent state prior to translocation. For example, one of the following combinations may or may not be chosen—

SSA1 in combination with one of SSA2, SSA3, SSA4, SSE1, SSE2, SSB1, SSB2;
SSA2 in combination with one of SSA3, SSA4, SSE1, SSE2, SSB1, SSB2;
SSA3 in combination with one of SSA4, SSE1, SSE2, SSB1, SSB2;
SSA4 in combination with one of SSE1, SSE2, SSB1, SSB2;
SSE1 in combination with one of SSE2, SSB1, SSB2;
SSE2 in combination with one of SSB1, SSB2; or
SSB1 in combination with SSB2.

In another embodiment, the host cell may or may not be genetically modified to cause over-expression of three of the above chaperones involved in cytoplasmic folding and maintenance of proteins in a translocation competent state prior to translocation. For example, one of the following combinations may or may not be chosen—

SSA1, SSA2 and SSA3; SSA1, SSA2 and SSA4; SSA1, SSA2 and SSE1; SSA1, SSA2 and SSE2; SSA1, SSA2 and SSB1; SSA1, SSA2 and SSB2; SSA1, SSA3 and SSA4; SSA1, SSA3 and SSE1; SSA1, SSA3 and SSE2; SSA1, SSA3 and SSB1; SSA1, SSA3 and SSB2; SSA1, SSA4 and SSE1; SSA1, SSA4 and SSE2; SSA1, SSA4 and SSB1; SSA1, SSA4 and SSB2; SSA1, SSE1 and SSE2; SSA1, SSE1 and SSB1; SSA1, SSE1 and SSB2; SSA1, SSE2 and SSB1; SSA1, SSE2 and SSB2; SSA1, SSB1 and SSB2; SSA2, SSA3 and SSA4; SSA2, SSA3 and SSE1; SSA2, SSA3 and SSE2; SSA2, SSA3 and SSB1; SSA2, SSA3 and SSB2; SSA2, SSA4 and SSE1; SSA2, SSA4 and SSE2; SSA2, SSA4 and SSB1; SSA2, SSA4 and SSB2; SSA2, SSE1 and SSE2; SSA2, SSE1 and SSB1; SSA2, SSE1 and SSB2; SSA2, SSE2 and SSB1; SSA2, SSE2 and SSB2; SSA2, SSB1 and SSB2; SSA3, SSA4 and SSE1; SSA3, SSA4 and SSE2; SSA3, SSA4 and SSB1; SSA3, SSA4 and SSB2; SSA3, SSE1 and SSE2; SSA3, SSE1 and SSB1; SSA3, SSE1 and SSB2; SSA3, SSE2 and SSB1; SSA3, SSE2 and SSB2; SSA3, SSB1 and SSB2; SSA4, SSE1 and SSE2; SSA4, SSE1 and SSB1; SSA4, SSE1 and SSB2; SSA4, SSE2 and SSB1; SSA4, SSE2 and SSB2; SSA4, SSB1 and SSB2; SSE1, SSE2 and SSB1; SSE1, SSE2 and SSB2; SSE1, SSB1 and SSB2; or SSE2, SSB1 and SSB2.

In another embodiment, the host cell mayor may not be genetically modified to cause over-expression of four of the above chaperones involved in cytoplasmic folding and maintenance of proteins in a translocation competent state prior to translocation. For example, one of the following combinations may or may not be chosen—

SSA1, SSA2, SSA3 and SSA4; SSA1, SSA2, SSA3 and SSE1; SSA1, SSA2, SSA3 and SSE2; SSA1, SSA2, SSA3 and SSB1; SSA1, SSA2, SSA3 and SSB2; SSA1, SSA2, SSA4 and SSE1; SSA1, SSA2, SSA4 and SSE2; SSA1, SSA2, SSA4 and SSB1; SSA1, SSA2, SSA4 and SSB2; SSA1, SSA2, SSE1 and SSE2; SSA1, SSA2, SSE1 and SSB1; SSA1, SSA2, SSE1 and SSB2; SSA1, SSA2, SSE2 and SSB1; SSA1, SSA2, SSE2 and SSB2; SSA1, SSA2, SSB1 and SSB2; SSA1, SSA3, SSA4 and SSE1; SSA1, SSA3, SSA4 and SSE2; SSA1, SSA3, SSA4 and SSB1; SSA1, SSA3, SSA4 and SSB2; SSA1, SSA3, SSE1 and SSE2; SSA1, SSA3, SSE1 and SSB1; SSA1, SSA3, SSE1 and SSB2; SSA1, SSA3, SSE2 and SSB1; SSA1, SSA3, SSE2 and SSB2; SSA1, SSA3, SSB1 and SSB2; SSA1, SSA4, SSE1 and SSE2; SSA1, SSA4, SSE1 and SSB1; SSA1, SSA4, SSE1 and SSB2; SSA1, SSA4, SSE2 and SSB1; SSA1, SSA4, SSE2 and SSB2; SSA1, SSA4, SSB1 and SSB2; SSA1, SSE1, SSE2 and SSB1; SSA1, SSE1, SSE2 and SSB2; SSA1, SSE1, SSB1 and SSB2; SSA1, SSE2, SSB1 and SSB2; SSA2, SSA3, SSA4 and SSE1; SSA2, SSA3, SSA4 and SSE2; SSA2, SSA3, SSA4 and SSB1; SSA2, SSA3, SSA4 and SSB2; SSA2, SSA3, SSE1 and SSE2; SSA2, SSA3, SSE1 and SSB1; SSA2, SSA3, SSE1 and SSB2; SSA2, SSA3, SSE2 and SSB1; SSA2, SSA3, SSE2 and SSB2; SSA2, SSA3, SSB1 and SSB2; SSA2, SSA4, SSE1 and SSE2; SSA2, SSA4, SSE1 and SSB1; SSA2, SSA4, SSE1 and SSB2; SSA2, SSA4, SSE2 and SSB1; SSA2, SSA4, SSE2 and SSB2; SSA2, SSA4, SSB1 and SSB2; SSA2, SSE1, SSE2 and SSB1; SSA2, SSE1, SSE2 and SSB2; SSA2, SSE1, SSB1 and SSB2; SSA2, SSE2, SSB1 and SSB2; SSA3, SSA4, SSE1 and SSE2; SSA3, SSA4, SSE1 and SSB1; SSA3, SSA4, SSE1 and SSB2; SSA3, SSA4, SSE2 and SSB1; SSA3, SSA4, SSE2 and SSB2; SSA3, SSA4, SSB1 and SSB2; SSA3, SSE1, SSE2 and SSB1; SSA3, SSE1, SSE2 and SSB2; SSA3, SSE1, SSB1 and SSB2; SSA3, SSE2, SSB1 and SSB2; SSA4, SSE1, SSE2 and SSB1; SSA4, SSE1, SSE2 and SSB2; SSA4, SSE1, SSB1 and SSB2; SSA4, SSE2, SSB1 and SSB2; or SSE1, SSE2, SSB1 and SSB2.

In another embodiment, the host cell may or may not be genetically modified to cause over-expression of five of the above chaperones involved in cytoplasmic folding and maintenance of proteins in a translocation competent state prior to translocation. For example, one of the following combinations may or may not be chosen—

SSA1, SSA2, SSA3, SSA4 and SSE1; SSA1, SSA2, SSA3, SSA4 and SSE2; SSA1, SSA2, SSA3, SSA4 and SSB1; SSA1, SSA2, SSA3, SSA4 and SSB2; SSA1, SSA2, SSA3, SSE1 and SSE2; SSA1, SSA2, SSA3, SSE1 and SSB1; SSA1, SSA2, SSA3, SSE1 and SSB2; SSA1, SSA2, SSA3, SSE2 and SSB1; SSA1, SSA2, SSA3, SSE2 and SSB2; SSA1, SSA2, SSA3, SSB1 and SSB2; SSA1, SSA2, SSA4, SSE1 and SSE2; SSA1, SSA2, SSA4, SSE1 and SSB1; SSA1, SSA2, SSA4, SSE1 and SSB2; SSA1, SSA2, SSA4, SSE2 and SSB1; SSA1, SSA2, SSA4, SSE2 and SSB2; SSA1, SSA2, SSA4, SSB1 and SSB2; SSA1, SSA2, SSE1, SSE2 and SSB1; SSA1, SSA2, SSE1, SSE2 and SSB2; SSA1, SSA2, SSE1, SSB1 and SSB2; SSA1, SSA2, SSE2, SSB1 and SSB2; SSA1, SSA3, SSA4, SSE1 and SSE2; SSA1, SSA3, SSA4, SSE1 and SSB1; SSA1, SSA3, SSA4, SSE1 and SSB2; SSA1, SSA3, SSA4, SSE2 and SSB1; SSA1, SSA3, SSA4, SSE2 and SSB2; SSA1, SSA3, SSA4, SSB1 and SSB2; SSA1, SSA3, SSE1, SSE2 and SSB1; SSA1, SSA3, SSE1, SSE2 and SSB2; SSA1, SSA3, SSE1, SSB1 and SSB2; SSA1, SSA3, SSE2, SSB1 and SSB2; SSA1, SSA4, SSE1, SSE2 and SSB1; SSA1, SSA4, SSE1, SSE2 and SSB2; SSA1, SSA4, SSE1, SSB1 and SSB2; SSA1, SSE1, SSE2, SSB1 and SSB2; SSA2, SSA3, SSA4, SSE1 and SSE2; SSA2, SSA3, SSA4, SSE1 and SSB1; SSA2, SSA3, SSA4, SSE1 and SSB2; SSA2, SSA3, SSA4, SSE2 and SSB1; SSA2, SSA3, SSA4, SSE2 and SSB2; SSA2, SSA3, SSA4, SSB1 and SSB2; SSA2, SSA3, SSE1, SSE2 and SSB1; SSA2, SSA3, SSE1, SSE2 and SSB2; SSA2, SSA3, SSE1, SSB1 and SSB2; SSA2, SSA3, SSE2, SSB1 and SSB2; SSA2, SSA4, SSE1, SSE2 and SSB1; SSA2, SSA4, SSE1, SSE2 and SSB2; SSA2, SSA4, SSE1, SSB1 and SSB2; SSA2, SSA4, SSE2, SSB1 and SSB2; SSA2, SSE1, SSE2, SSB1 and SSB2; SSA3, SSA4, SSE1, SSE2 and SSB1; SSA3, SSA4, SSE1, SSE2 and SSB2; SSA3, SSA4, SSE1, SSB1 and SSB2; SSA3, SSA4, SSE2, SSB1 and SSB2; SSA3, SSE1, SSE2, SSB1 and SSB2; or SSA4, SSE1, SSE2, SSB1 and SSB2.

In another embodiment, the host cell may or may not be genetically modified to cause over-expression of six of the above chaperones involved in cytoplasmic folding and maintenance of proteins in a translocation competent state prior to translocation. For example, one of the following combinations may or may not be chosen—

SSA1, SSA2, SSA3, SSA4, SSE1 and SSE2; SSA1, SSA2, SSA3, SSA4, SSE1 and SSB1; SSA1, SSA2, SSA3, SSA4, SSE1 and SSB2; SSA1, SSA2, SSA3, SSA4, SSE2 and SSB1; SSA1, SSA2, SSA3, SSA4, SSE2 and SSB2; SSA1, SSA2, SSA3, SSA4, SSB1 and SSB2; SSA1, SSA2, SSA3, SSE1, SSE2 and SSB1; SSA1, SSA2, SSA3, SSE1, SSE2 and SSB2; SSA1, SSA2, SSA3, SSE1, SSB1 and SSB2; SSA1, SSA2, SSA3, SSE2, SSB1 and SSB2; SSA1, SSA2, SSA4, SSE1, SSE2 and SSB1; SSA1, SSA2, SSA4, SSE1, SSE2 and SSB2; SSA1, SSA2, SSA4, SSE1, SSB1 and SSB2; SSA1, SSA2, SSA4, SSE2, SSB1 and SSB2; SSA1, SSA2, SSE1, SSE2, SSB1 and SSB2; SSA1, SSA3, SSA4, SSE1, SSE2 and SSB1; SSA1, SSA3, SSA4, SSE1, SSE2 and SSB2; SSA1, SSA3, SSA4, SSE1, SSB1 and SSB2; SSA1, SSA3, SSA4, SSE2, SSB1 and SSB2; SSA1, SSA3, SSE1, SSE2, SSB1 and SSB2; SSA1, SSA4, SSE1, SSE2, SSB1 and SSB2; SSA2, SSA3, SSA4, SSE1, SSE2 and SSB1; SSA2, SSA3, SSA4, SSE1, SSE2 and SSB2; SSA2, SSA3, SSA4, SSE1, SSB1 and SSB2; SSA2, SSA3, SSA4, SSE2, SSB1 and SSB2; SSA2, SSA3, SSE1, SSE2, SSB1 and SSB2; SSA2, SSA4, SSE1, SSE2, SSB1 and SSB2; or SSA3, SSA4, SSE1, SSE2, SSB1 and SSB2.

In another embodiment, the host cell may or may not be genetically modified to cause over-expression of seven of the above chaperones involved in cytoplasmic folding and maintenance of proteins in a translocation competent state prior to translocation. For example, one of the following combinations may or may not be chosen—

SSA1, SSA2, SSA3, SSA4, SSE1, SSE2 and SSB1; SSA1, SSA2, SSA3, SSA4, SSE1, SSE2 and SSB; SSA1, SSA2, SSA3, SSA4, SSE1, SSB1 and SSB2; SSA1, SSA2; SSA3, SSA4, SSE2, SSB1 and SSB2; SSA1, SSA2, SSA3, SSE1, SSE2, SSB1 and SSB2; SSA1, SSA2, SSA4, SSE1, SSE2, SSB1 and SSB2; SSA1, SSA3, SSA4, SSE1, SSE2, SSB1 and SSB2; or SSA2, SSA3, SSA4, SSE1, SSE2, SSB1 and SSB2.

In another embodiment, the host cell may or may not be genetically modified to cause over-expression of all eight of the above chaperones involved in cytoplasmic folding and maintenance of proteins in a translocation competent state prior to translocation. In other words, the following combination may or may not be chosen—

### SSA1, SSA2, SSA3, SSA4, SSE1, SSE2, SSB1 and SSB2.

### Mitochondrial Chaperone and Translocation Proteins

Mitochondrial chaperone and translocation proteins include ECM10, MDJ1, MDJ2. A detailed description of these proteins and their genes is given separately below.

In one embodiment, the host cell may or may not be genetically modified to cause over-expression of one of the above mitochondrial chaperone and translocation proteins.

In another embodiment, the host cell may or may not be genetically modified to cause over-expression of two of the above mitochondrial chaperone and translocation proteins. For example, one of the following combinations may or may not be chosen—

### ECM10 and MDJ1; ECM10 and MDJ2; or MDJ1 and MDJ2.

In another embodiment, the host cell may or may not be genetically modified to cause over-expression of all three of the above mitochondrial chaperone and translocation proteins. In that case the following combination may or may not be chosen—

### ECM10, MDJ1 and MDJ2.

### Other Combinations of Chaperones

The skilled person will appreciate that it is possible to combine genes that encode one or more proteins from the above-defined groups of chaperones.

Thus, the host cell may or may not be genetically modified to cause simultaneous over-expression of at least one, two, three, four, five, six, seven, eight, nine, ten, eleven, twelve, thirteen, fourteen, fifteen, sixteen or seventeen of the chaperones selected from the group consisting of JEM1, LHS1, SCJ1, KAR2, SIL1 FKB2, SSA1, SSA2, SSA3, SSA4, SSE1, SSE2, SSB1, SSB2, ECM10, MDJ1 and MDJ2.

Where the host cell is genetically modified to cause simultaneous over-expression of one or two of the above defined chaperones, it may or may not be preferred that the host cell is genetically modified to cause simultaneous over-expression of at least three helper proteins and the one or two other helper proteins may or may not be helper proteins involved in disulphide bond formation or protein degradation, as discussed below.

Over-expression of one (or more) of the ER luminal localised chaperones may or may not be combined with the over-expression of at least one of the chaperones involved in cytoplasmic folding and maintenance of proteins in a translocation competent state prior to translocation and/or the over-expression of at least one of the mitochondrial chaperone and translocation proteins.

For example, any one of the following combinations may or may not be chosen—
SCJ1 in combination with any one, two, three, four, five, six, seven, eight, nine, ten or eleven of SSA1, SSA2, SSA3, SSA4, SSE1, SSE2, SSB1, SSB2, ECM10, MDJ1 or MDJ2; or
FKB2 in combination with any one, two, three, four, five, six, seven, eight, nine, ten or eleven of SSA1, SSA2, SSA3, SSA4, SSE1, SSE2, SSB1, SSB2, ECM10, MDJ1 or MDJ2.
JEM1 in combination with any of the above-listed combinations of one, two, three, four, five, six, seven or eight of SSA1, SSA2, SSA3, SSA4, SSE1, SSE2, SSB1, SSB2 and/or in combination with ECM10, MDJ1 and MDJ2;
LHS1 in combination with any of the above-listed combinations of one, two, three, four, five, six, seven or eight of SSA1, SSA2, SSA3, SSA4, SSE1, SSE2, SSB1, SSB2 and/or in combination with ECM10, MDJ1 and MDJ2;
SCJ1 in combination with any of the above-listed combinations of one, two, three, four, five, six, seven or eight of SSA1, SSA2, SSA3, SSA4, SSE1, SSE2, SSB1, SSB2 and/or in combination with ECM10, MDJ1 and MDJ2;
KAR2 in combination with any of the above-listed combinations of one, two, three, four, five, six, seven or eight of SSA1, SSA2, SSA3, SSA4, SSE1, SSE2, SSB1, SSB2 and/or in combination with ECM10, MDJ1 and MDJ2;
SIL1 in combination with any of the above-listed combinations of one, two, three, four, five, six, seven or eight of SSA1, SSA2, SSA3, SSA4, SSE1, SSE2, SSB1, SSB2 and/or in combination with ECM10, MDJ1 and MDJ2; or
FKB2 in combination with any of the above-listed combinations of one, two, three, four, five, six, seven or eight of SSA1, SSA2, SSA3, SSA4, SSE1, SSE2, SSB1, SSB2 and/or in combination with ECM10, MDJ1 and MDJ2.

Alternatively, for example, one (or more) of the chaperones involved in cytoplasmic folding and maintenance of proteins in a translocation competent state prior to translocation may or may not be simultaneously over-expressed with at least one of the ER luminal localised chaperones and/or at least one of the mitochondrial chaperone and translocation proteins. For example, the following combinations may or may not be chosen—
SSE1 in combination with any one, two, three, four, five, six, seven, eight or nine of JEM1, LHS1, SCJ1, KAR2, SIL1, FKB2, ECM10, MDJ1 or MDJ2.
SSA1 in combination with any of the above-listed combinations of one, two, three, four, five or six of JEM1, LHS1, SCJ1, KAR2, SIL1 and FKB2 and/or in combination with ECM10, MDJ1 and MDJ2;
SSA2 in combination with any of the above-listed combinations of one, two, three, four, five or six of JEM1, LHS1, SCJ1, KAR2, SIL1 and FKB2 and/or in combination with ECM10, MDJ1 and MDJ2;
SSA3 in combination with any of the above-listed combinations of one, two, three, four, five or six of JEM1, LHS1, SCJ1, KAR2, SIL1 and FKB2 and/or in combination with ECM10, MDJ1 and MDJ2;
SSA4 in combination with any of the above-listed combinations of one, two, three, four, five or six of JEM1, LHS1, SCJ1, KAR2, SIL1 and FKB2 and/or in combination with ECM10, MDJ1 and MDJ2;
SSE1 in combination with any of the above-listed combinations of one, two, three, four, five or six of JEM1, LHS1, SCJ1, KAR2, SIL1 and FKB2 and/or in combination with ECM10, MDJ1 and MDJ2;
SSE2 in combination with any of the above-listed combinations of one, two, three, four, five or six of JEM1, LHS1, SCJ1, KAR2, SIL1 and FKB2 and/or in combination with ECM10, MDJ1 and MDJ2;
SSB1 in combination with any of the above-listed combinations of one, two, three, four, five or six of JEM1, LHS1, SCJ1, KAR2, SIL1 and FKB2 and/or in combination with ECM10, MDJ1 and MDJ2; or
SSB2 in combination with any of the above-listed combinations of one, two, three, four, five or six of JEM1, LHS1, SCJ1, KAR2, SIL1 and FKB2 and/or in combination with ECM10, MDJ1 and MDJ2.

Alternatively, one of the mitochondrial chaperone and translocation proteins may or may not be simultaneously over-expressed with at least one of the chaperones involved in cytoplasmic folding and maintenance of proteins in a translocation competent state prior to translocation and/or at least one of the ER luminal localised chaperones.

For example, one of the following combinations may or may not be chosen—
ECM10 in combination with any of the above-listed combinations of one, two, three, four, five or six of JEM1, LHS1, SCJ1, KAR2, SIL1 and FKB2;
ECM10 in combination with any of the above-listed combinations of one, two, three, four, five, six, seven or eight of SSA1, SSA2, SSA3, SSA4, SSE1, SSE2, SSB1, SSB2;
ECM10 in combination with any of the above-listed combinations of one, two, three of JEM1, LHS1, SCJ1, KAR2, SIL1 and FKB2 and any of the above-listed combinations of one, two, three, four, five, six, seven or eight of SSA1, SSA2, SSA3, SSA4, SSE1, SSE2, SSB1, SSB2;
ECM10 in combination with any of the above-listed combinations of four of JEM1, LHS1, SCJ1, KAR2, SIL1 and FKB2 and any of the above-listed combinations of one, two, three, four, five, six, seven or eight of SSA1, SSA2, SSA3, SSA4, SSE1, SSE2, SSB1, SSB2;
ECM10 in combination with any of the above-listed combinations of five of JEM1, LHS1, SCJ1, KAR2, SILT and FKB2 and any of the above-listed combinations of one, two, three, four, five, six, seven or eight of SSA1, SSA2, SSA3, SSA4, SSE1, SSE2, SSB1, SSB2;
ECM10 in combination with all six of JEM1, LHS1, SCJ1, KAR2, SIL1 and FKB2 and any of the above-listed combinations of one, two, three, four, five, six, seven or eight of SSA1, SSA2, SSA3, SSA4, SSE1, SSE2, SSB1, SSB2;
MDJ1 in combination with any of the above-listed combinations of one, two, three, four, five or six of JEM1, LHS1, —SCJ1, KAR2, SIL1 and FKB2;
MDJ1 in combination with any of the above-listed combinations of one, two, three, four, five, six, seven or eight of SSA1, SSA2, SSA3, SSA4, SSE1, SSE2, SSB1, SSB2;
MDJ1 in combination with any of the above-listed combinations of one, two, three of JEM1, LHS1, SCJ1, KAR2, SIL1 and FKB2 and any of the above-listed combinations of one, two, three, four, five, six, seven or eight of SSA1, SSA2, SSA3, SSA4, SSE1, SSE2, SSB1, SSB2;
MDJ1 in combination with any of the above-listed combinations of four of JEM1, LHS1, SCJ1, KAR2, SIL1 and FKB2 and any of the above-listed combinations of one, two, three, four, five, six, seven or eight of SSA1, SSA2, SSA3, SSA4, SSE1, SSE2, SSB1, SSB2;
MDJ1 in combination with any of the above-listed combinations of five of JEM1, LHS1, SCJ1, KAR2, SIL1 and FKB2 and any of the above-listed combinations of one, two, three, four, five, six, seven or eight of SSA1, SSA2, SSA3, SSA4, SSE1, SSE2, SSB1, SSB2;
MDJ1 in combination with all six of JEM1, LHS1, SCJ1, KAR2, SIL1 and FKB2 and any of the above-listed combinations of one, two, three, four, five, six, seven or eight of SSA1, SSA2, SSA3, SSA4, SSE1, SSE2, SSB1, SSB2;
MDJ2 in combination with any of the above-listed combinations of one, two, three, four, five or six of JEM1, LHS1, SCJ1, KAR2, SIL1 and FKB2;
MDJ2 in combination with any of the above-listed combinations of one, two, three, four, five, six, seven or eight of SSA1, SSA2, SSA3, SSA4, SSE1, SSE2, SSB1, SSB2;
MDJ2 in combination with any of the above-listed combinations of one, two, three of JEM1, LHS1, SCJ1, KAR2, SIL1 and FKB2 and any of the above-listed combinations of one, two, three, four, five, six, seven or eight of SSA1, SSA2, SSA3, SSA4, SSE1, SSE2, SSB1, SSB2;
MDJ2 in combination with any of the above-listed combinations of four of JEM1, LHS1, SCJ1, KAR2, SIL1 and FKB2 and any of the above-listed combinations of one, two, three, four, five, six, seven or eight of SSA1, SSA2, SSA3, SSA4, SSE1, SSE2, SSB1, SSB2;
MDJ2 in combination with any of the above-listed combinations of five of JEM1, LHS1, SCJ1, KAR2, SIL1 and FKB2 and any of the above-listed combinations of one, two, three, four, five, six, seven or eight of SSA1, SSA2, SSA3, SSA4, SSE1, SSE2, SSB1, SSB2; or
MDJ2 in combination with all six of JEM1, LHS1, SCJ1, KAR2, SIL1 and FKB2 and any of the above-listed combinations of one, two, three, four, five, six, seven or eight of SSA1, SSA2, SSA3, SSA4, SSE1, SSE2, SSB1, SSB2.

In another embodiment, representative members of each of the above three groups of chaperone proteins (such as one member of each group) may or may not be simultaneously over-expressed in the host cell. For example, one of the following combinations may or may not be chosen—

JEM1, SSA1 and ERM10; JEM1, SSA1 and MDJ1; JEM1, SSA1 and MDJ2; JEM1, SSA2 and ERM10; JEM1, SSA2 and MDJ1; JEM1, SSA2 and MDJ2; JEM1, SSA3 and ERM10; JEM1, SSA3 and MDJ1; JEM1, SSA3 and MDJ2; JEM1, SSA4 and ERM10; JEM1, SSA4 and MDJ1; JEM1, SSA4 and MDJ2; JEM1, SSE1 and ERM10; JEM1, SSE1 and MDJ1; JEM1, SSE1 and MDJ2; JEM1, SSE2 and ERM10; JEM1, SSE2 and MDJ1; JEM1, SSE2 and MDJ2; JEM1, SSB1 and ERM10; JEM1, SSB1 and MDJ1; JEM1, SSB1 and MDJ2; JEM1, SSB2 and ERM10; JEM1, SSB2 and MDJ1; JEM1, SSB2 and MDJ2; LHS1, SSA1 and ERM10; LHS1, SSA1 and MDJ1; LHS1, SSA1 and MDJ2; LHS1, SSA2 and ERM10; LHS1, SSA2 and MDJ1; LHS1, SSA2 and MDJ2; LHS1, SSA3 and ERM10; LHS1, SSA3 and MDJ1 LHS1, SSA3 and MDJ2; LHS1, SSA4 and ERM10; LHS1, SSA4 and MDJ1; LHS1, SSA4 and MDJ2; LHS1, SSE1 and ERM10; LHS1, SSE1 and MDJ1; LHS1, SSE1 and MDJ2; LHS1, SSE2 and ERM10; LHS1, SSE2 and MDJ1; LHS1, SSE2 and MDJ2; LHS1, SSB1 and ERM10; LHS1, SSB1 and MDJ1; LHS1, SSB1 and MDJ2; LHS1, SSB2 and ERM10; LHS1, SSB2 and MDJ1; LHS1, SSB2 and MDJ2; SCJ1, SSA1 and ERM10; SCJ1, SSA1 and MDJ1; SCJ1, SSA1 and MDJ2; SCJ1, SSA2 and ERM10; SCJ1, SSA2 and MDJ1; SCJ1, SSA2 and MDJ2; SCJ1, SSA3 and ERM10; SCJ1, SSA3 and MDJ1; SCJ1, SSA3 and MDJ2; SCJ1, SSA4 and ERM10; SCJ1, SSA4 and MDJ1; SCJ1, SSA4 and MDJ2; SCJ1, SSE1 and ERM10; SCJ1, SSE1 and MDJ1; SCJ1, SSE1 and MDJ2; SCJ1, SSE2 and ERM10; SCJ1, SSE2 and MDJ1; SCJ1, SSE2 and MDJ2; SCJ1, SSB1 and ERM10; SCJ1, SSB1 and MDJ1; SCJ1, SSB1 and MDJ2; SCJ1, SSB2 and ERM10; SCJ1, SSB2 and MDJ1; SCJ1, SSB2 and MDJ2; KAR2, SSA1 and ERM10; KAR2, SSA1 and MDJ1; KAR2, SSA1 and MDJ2; KAR2, SSA2 and ERM10; KAR2, SSA2 and MDJ1; KAR2, SSA2 and MDJ2; KAR2, SSA3 and ERM10; KAR2, SSA3 and MDJ1; KAR2, SSA3 and MDJ2; KAR2, SSA4 and ERM10; KAR2, SSA4 and MDJ1; KAR2, SSA4 and MDJ2; KAR2, SSE1 and ERM10; KAR2, SSE1 and MDJ1; KAR2, SSE1 and MDJ2; KAR2, SSE2 and ERM10; KAR2, SSE2 and MDJ1; KAR2, SSE2 and MDJ2; KAR2, SSB1 and ERM10; KAR2, SSB1 and MDJ1; KAR2, SSB1 and MDJ2; KAR2, SSB2 and ERM10; KAR2, SSB2 and MDJ1; KAR2, SSB2 and MDJ2; SIL1 SSA1 and ERM10; SIL1 SSA1 and MDJ1; SIL1 SSA1 and MDJ2; SSA2 and ERM10; SIL1 SSA2 and MDJ1; SIL1 SSA2 and MDJ2; SIL1, SSA3 and ERM10; SIL1 SSA3 and MDJ1; SIL1 SSA3 and MDJ2; SIL1 SSA4 and ERM10; SIL1 SSA4 and MDJ1; SIL1 SSA4 and MDJ2; SIL1, SSE1 and ERM10; SIL1, SSE1 and MDJ1; SIL1, SSE1 and MDJ2; SIL1, SSE2 and ERM10; SIL1, SSE2 and MDJ1; SIL1, SSE2 and MDJ2; SIL1, SSB1 and ERM10; SIL1, SSB1 and MDJ1; SIL1, SSB1 and MDJ2; SIL1, SSB2 and ERM10; SIL1, SSB2 and MDJ1; SIL1, SSB2 and MDJ2; FKB2, SSA1 and ERM10; FKB2, SSA1 and MDJ1; FKB2, SSA1 and MDJ2; FKB2, SSA2 and ERM10; FKB2, SSA2 and MDJ1; FKB2, SSA2 and MDJ2; FKB2, SSA3 and ERM10; FKB2, SSA3 and MDJ1; FKB2, SSA3 and MDJ2; FKB2, SSA4 and ERM10; FKB2, SSA4 and MDJ1; FKB2, SSA4 and MDJ2; FKB2, SSE1 and ERM10; FKB2, SSE1 and MDJ1; FKB2, SSE1 and MDJ2; FKB2, SSE2 and ERM10; FKB2, SSE2 and MDJ1; FKB2, SSE2 and MDJ2; FKB2, SSB1 and ERM10; FKB2, SSB1 and MDJ1; FKB2, SSB1 and MDJ2; FKB2, SSB2 and ERM10; FKB2, SSB2 and MDJ1; or FKB2, SSB2 and MDJ2.

The skilled person will also appreciate that any of the above defined combinations may or may not also be combined with any of the following genes or combinations of genes encoding other helper proteins, in particular helper proteins involved in disulphide bond formation or helper proteins involved in protein degradation, as discussed below.

### Proteins Involved in Disulphide Bond Formation

Proteins involved in the formation of disulphide bonds in other proteins include ERO1, ERV2, EUG1, MPD1, MPD2, EPS1 and PDI1. A detailed description of these proteins and their genes is given separately below.

In one embodiment, one of the above disulphide bond formation proteins may or may not be over-expressed in the host cell. For example, ERV2 may or may not be chosen.

In another embodiment, two of the above disulphide bond formation proteins may or may not be simultaneously over-expressed in the host cell. For example, one of the following combinations may or may not be chosen—
ERO1 in combination with one of ERV2, EUG1, MPD1, MPD2, EPS1 or PDI1;
ERV2 in combination with one of EUG1, MPD1, MPD2, EPS1 or PDI1;
EUG1 in combination with one of MPD1, MPD2, EPS1 or PDI1;
MPD1 in combination with one of MPD2, EPS1 or PDI1;
MPD2 in combination with one of EPS1 or PDI1; or
EPS1 in combination with PDI1.

In another embodiment, three of the above helper proteins may or may not be simultaneously over-expressed in the host cell. For example, one of the following combinations may or may not be chosen—

ERO1, ERV2 and EUG1; ERO1, ERV2 and MPD1; ERO1, ERV2 and MPD2; ERO1, ERV2 and EPS1; ERO1, ERV2 and PDI1; ERO1, EUG1 and MPD1; ERO1, EUG1 and MPD2; ERO1, EUG1 and EPS1; ERO1, EUG1 and PDI1; ERO1, MPD1 and MPD2; ERO1, MPD1 and EPS1; ERO1, MPD1 and PDI1; ERO1, MPD2 and EPS1; ERO1, MPD2 and PDI1; ERO1, EPS1 and PDI1; ERV2, EUG1 and MPD1; ERV2, EUG1 and MPD2; ERV2, EUG1 and EPS1; ERV2, EUG1 and PDI1; ERV2, MPD1 and MPD2; ERV2, MPD1 and EPS1; ERV2, MPD1 and PDI1; ERV2, MPD2 and EPS1; ERV2, MPD2 and PDI1; ERV2, EPS1 and PDI1; EUG1, MPD1 and MPD2; EUG1, MPD1 and EPS1; EUG1, MPD1 and PDI1; EUG1, MPD2 and EPS1; EUG1, MPD2 and PDI1; EUG1, EPS1 and PDI1; MPD1, MPD2 and EPS1; MPD1, MPD2 and PDI1; MPD1, EPS1 and PDI1; or MPD2; EPS1 and PDI1.

In another embodiment, four of the above helper proteins may or may not be simultaneously over-expressed in the host cell. For example, one of the following combinations may or may not be chosen—

ERO1, ERV2, EUG1 and MPD1; ERO1, ERV2, EUG1 and MPD2; ERO1, ERV2, EUG1 and EPS1; ERO1, ERV2, EUG1 and PDI1; ERO1, ERV2, MPD1 and MPD2; ERO1, ERV2, MPD1 and EPS1; ERO1, ERV2, MPD1 and PDI1; ERO1, ERV2, MPD2 and EPS1; ERO1, ERV2, MPD2 and PDI1; ERO1, ERV2, EPS1 and PDI1; ERO1, EUG1, MPD1 and MPD2; ERO1, EUG1, MPD1 and EPS1; ERO1, EUG1, MPD1 and PDI1; ERO1, EUG1, MPD2 and EPS1; ERO1, EUG1, MPD2 and PDI1; ERO1, EUG1, EPS1 and PDI1; ERO1, MPD1, MPD2 and EPS1; ERO1, MPD1, MPD2 and PDI1; ERO1, MPD1, EPS1 and PDI1; ERO1, MPD2, EPS1 and PDI1; ERV2, EUG1, MPD1 and MPD2; ERV2, EUG1, MPD1 and EPS1; ERV2, EUG1, MPD1 and PDI1; ERV2, EUG1, MPD2 and EPS1; ERV2, EUG1, MPD2 and PDI1; ERV2, EUG1, EPS1 and PDI1; ERV2, MPD1, MPD2 and EPS1; ERV2, MPD1, MPD2 and PDI1; ERV2, MPD1, EPS1 and PDI1; ERV2, MPD2, EPS1 and PDI1; EUG1, MPD1, MPD2 and EPS1; EUG1, MPD1, MPD2 and PDI1; EUG1, MPD1, EPS1 and PDI1; EUG1, MPD2, EPS1 and PDI1; or MPD1, MPD2, EPS1 and PDI1.

In another embodiment, five of the above helper proteins may or may not be simultaneously over-expressed in the host cell. For example, one of the following combinations may or may not be chosen—

ERO1, ERV2, EUG1, MPD1 and MPD2; ERO1, ERV2, EUG1, MPD1 and EPS1; ERO1, ERV2, EUG1, MPD1 and PDI1; ERO1, ERV2, EUG1, MPD2 and EPS1; ERO1, ERV2, EUG1, MPD2 and PDI1; ERO1, ERV2, EUG1, EPS1 and PDI1; ERO1, ERV2, MPD1, MPD2 and EPS1; ERO1, ERV2, MPD1, MPD2 and PDI1; ERO1, ERV2, MPD1, EPS1 and PDI1; ERO1, ERV2, MPD2, EPS1 and PDI1; ERO1, EUG1, MPD1, MPD2 and EPS1; ERO1, EUG1, MPD1, MPD2 and PDI1; ERO1, EUG1, MPD1, EPS1 and PDI1; ERO1, EUG1, MPD2, EPS1 and PDI1; ERO1, MPD1, MPD2, EPS1 and PDI1; ERV2, EUG1, MPD1, MPD2 and EPS1; ERV2, EUG1, MPD1, MPD2 and PDI1; ERV2, EUG1, MPD1, EPS1 and PDI1; ERV2, EUG1, MPD2, EPS1 and PDI1; ERV2, MPD1, MPD2, EPS1 and PDI1; or EUG1, MPD1, MPD2, EPS1 and PDI1

In another embodiment, six of the above helper proteins may or may not be simultaneously over-expressed in the host cell. For example, one of the following combinations may or may not be chosen—

ERO1, ERV2, EUG1, MPD1, MPD2 and EPS1; ERO1, ERV2, EUG1, MPD1, MPD2 and PDI1; ERO1, ERV2, EUG1, MPD1, EPS1 and PDI1; ERO1, ERV2, EUG1, MPD2, EPS1 and PDI1; ERO1, ERV2, MPD1, MPD2, EPS1 and PDI1; ERO1, EUG1, MPD1, MPD2, EPS1 and PDI1; or ERV2, EUG1, MPD1, MPD2, EPS1 and PDI1.

It is anticipated that ERO1 and ERV2 may function independently of each other or they may co-operate. Therefore, in one embodiment disclosure of ERO1 may or may not also include the combinations of ERO1 and ERV2, or ERV2 in its place. Similarly, in another embodiment disclosure of ERV2 may or may not also include the combinations of ERV2 and ERO1, or ERO1 in its place.

In another embodiment, all seven of the above helper proteins may or may not be simultaneously over-expressed in the host cell. In that case, the following combinations may or may not be chosen—

### ERO1, ERV2, EUG1, MPD1, MPD2, EPS1 and PDI1.

Where the host cell is genetically modified to cause simultaneous over-expression of one or two of the above defined disulphide bond formation helper proteins, it may or may not be preferred that the host cell is genetically modified to cause simultaneous over-expression of at least three helper proteins and the one or two other helper proteins may or may not be chaperones or helper proteins involved in protein degradation, as discussed above, and below, respectively.

Where one of the helper proteins is a protein disulphide isomerase, such as a yeast and mammalian PDI, mammalian Erp59, mammalian prolyl-4-hydroxylase B-subunit, yeast GSBP, yeast EUG1 and mammalian T3BP, then it may or may not be preferred, in one embodiment, to avoid co-expression with KAR2 or an equivalent thereof including hsp chaperone proteins such as other yeast Hsp70 proteins, BiP, SSA1-4, SSB1, SSC1 and SSD1 gene products and eukaryotic hsp70 proteins such as HSP68, HSP72, HSP73, HSC70, clathrin uncoating ATPase, IgG heavy chain binding protein (BiP), glucose-regulated proteins 75, 78 and 80 (GRP75, GPR78 and GRP80) and the like, particularly where these are the sole helper proteins that are overexpressed in the host cell.

### Proteins Involved in Protein Degradation

Proteins involved in protein degradation include DER1, DER3, HRD3, UBC7 and DOA4. A detailed description of these proteins and their genes is given separately below.

In one embodiment, one of the above proteins involved in protein degradation may or may not be over-expressed in the host cell. For example, DER1 may or may not be chosen, DER3 may or may not be chosen, HRD3 may or may not be chosen, UBC7 may or may not be chosen, or DOA4 may or may not be chosen.

In another embodiment, two of the above proteins involved in protein degradation may or may not be simultaneously over-expressed in the host cell. For example, one of the following combinations may or may not be chosen—

### DER1 and DER3; DER1 and HRD3; DER1 and UBC7; DER1 and DOA4; DER3 and HRD3; DER3 and UBC7; DER3 and DOA4; HRD3 and UBC7; HRD3 and DOA4; or UBC7 and DOA4.

In another embodiment, three of the above proteins involved in protein degradation may or may not be simultaneously over-expressed in the host cell. For example, one of the following combinations may or may not be chosen—

DER1, DER3 and HRD3; DER1, DER3 and UBC7; DER1, DER3 and DOA4; DER1, HRD3 and UBC7; DER1, HRD3 and DOA4; DER1, UBC7 and DOA4; DER3, HRD3 and UBC7; DER3, HRD3 and DOA4; DER3, UBC7 and DOA4; or HRD3, UBC7 and DOA4.

In another embodiment, four of the above proteins involved in protein degradation may or may not be simultaneously over-expressed in the host cell. For example, one of the following combinations may or may not be chosen—

### DER1, DER3, HRD3 and UBC7; DER1, DER3, HRD3 and DOA4; DER1, DER3, UBC7 and DOA4; DER1, HRD3, UBC7 and DOA4; or DER3, HRD3, UBC7 and DOA4.

In another embodiment, all five of the above proteins involved in protein degradation may or may not be simultaneously over-expressed in the host cell. In that case, the following combination is chosen—

### DER1, DER3, HRD3, UBC7 and DOA4.

Where the host cell is genetically modified to cause simultaneous over-expression of one or two of the above defined protein degradation helper proteins, it may or may not be preferred that the host cell is genetically modified to cause simultaneous over-expression of at least three helper proteins in total and the one or two other helper proteins may or may not be chaperones or disulphide bond formation helper proteins, as discussed above.

### HAC1 (Encoded by a Spliced or Unspliced Polynucleotide)

Valkonen et al. 2003 (Applied Environ. Micro., 69, 2065) reported investigations into the possibility to obtain better yields of secreted proteins. The authors found that the manipulation of the unfolded-protein response (UPR) pathway regulator, HAC1, affected the production of both native and foreign proteins in the yeast Saccharomyces cerevisiae. For example, it is reported that constitutive over-expression of HAC1 caused a 70% increase in alpha-amylase secretion. WO 01/72783 also reports that. HAC1 overexpression can be used to increase the secretion of a heterologous protein in a eukaryotic cell by inducing an elevated UPR, and PTC2 and IRE1 are also suggested for use in place of HAC1.

Over-expression of HAC1 can be achieved, for example, by the introduction of a recombinant polynucleotide that comprises the endogenous. HAC1 gene coding sequence or a truncated intronless HAC1 coding sequence (Valkonen et al. 2003, Applied Environ. Micro., 69, 2065). A detailed description of this protein and its gene is given separately below. The same techniques can be used to over-express PTC2 or IRE1.

In one embodiment of the present invention, a host cell of the present invention may or may not be genetically engineered to cause over-expression HAC1, PTC2 or IRE1, such as by modification of an endogenous gene encoding HAC1, PTC2 or IRE1, or by transformation with a recombinant gene encoding HAC1, PTC2 or IRE1. For example HAC1, PTC2 or IRE1 may or may not be simultaneously over-expressed with any of the above-defined combinations of other helper proteins.

In one embodiment, the host cell of the present invention is not genetically engineered to cause HAC1 over-expression, such as by modification of an endogenous HAC1 gene or transformation with a recombinant HAC1 gene.

In another embodiment where the host cell is genetically engineered to cause over-expression of HAC1, PTC2 or IRE1, the host cell is additionally genetically modified by the introduction of at least one recombinant gene encoding at least one other helper protein, such as a DnaJ-like protein, an Hsp70 family protein and/or SIL1 or by the modification of the sequence of an endogenous gene encoding one or more other helper proteins at least one of a DnaJ-like protein, an Hsp70 family protein (such as LHS1) and SIL1 to cause increased expression of the thus modified gene.

### Other Combinations

In light of the above disclosure, the skilled person will appreciate that the present invention also encompasses simultaneous over-expression of any combination of helper proteins derived from any of the above-defined groups.

For example, two helper proteins may or may not be simultaneously over-expressed. Suitable combinations include any one of the following combinations: JEM1 and LHS1; JEM1 and SCJ1; JEM1 and KAR2; JEM1 and SIL1; JEM1 and FKB2; JEM1 and SSA1; JEM1 and SSA2; JEM1 and SSA3; JEM1 and SSA4; JEM1 and SSE1; JEM1 and SSE2; JEM1 and SSB1; JEM1 and SSB2; JEM1 and ECM10; JEM1 and MDJ1; JEM1 and MDJ2; JEM1 and ERO1; JEM1 and ERV2; JEM1 and EUG1; JEM1 and MPD1; JEM1 and MPD2; JEM1 and EPS1; JEM1 and PDI1; JEM1 and DER1; JEM1 and DER3; JEM1 and HRD3; JEM1 and UBC7; JEM1 and DOA4; JEM1 and HAC1; LHS1 and SCJ1; LHS1 and KAR2; LHS1 and SIL1 LHS1 and FKB2; LHS1 and SSA1; LHS1 and SSA2; LHS1 and SSA3; LHS1 and SSA4; LHS1 and SSE1; LHS1 and SSE2; LHS1 and SSB1; LHS1 and SSB2; LHS1 and ECM10; LHS1 and MDJ1; LHS1 and MDJ2; LHS1 and ERO1; LHS1 and ERV2; LHS1 and EUG1; LHS1 and MPD1; LHS1 and MPD2; LHS1 and EPS1; LHS1 and PDI1; LHS1 and DER1; LHS1 and DER3; LHS1 and HRD3; LHS1 and UBC7; LHS1 and DOA4; LHS1 and HAC1; SCJ1 and KAR2; SCJ1 and SIL1; SCJ1 and FKB2; SCJ1 and SSA1; SCJ1 and SSA2; SCJ1 and SSA3; SCJ1 and SSA4; SCJ1 and SSE1; SCJ1 and SSE2; SCJ1 and SSB1; SCJ1 and SSB2; SCJ1 and ECM10; SCJ1 and MDJ1; SCJ1 and MDJ2; SCJ1 and ERO1; SCJ1 and ERV2; SCJ1 and EUG1; SCJ1 and MPD1; SCJ1 and MPD2; SCJ1 and EPS1; SCJ1 and PDI1; SCJ1 and DER1; SCJ1 and DER3; SCJ1 and HRD3; SCJ1 and UBC7; SCJ1 and DOA4; SCJ1 and HAC1; KAR2 and SIL1; KAR2 and FKB2; KAR2 and SSA1; KAR2 and SSA2; KAR2 and SSA3; KAR2 and SSA4; KAR2 and SSE1; KAR2 and SSE2; KAR2 and SSB1; KAR2 and SSB2; KAR2 and ECM10; KAR2 and MDJ1; KAR2 and MDJ2; KAR2 and ERO1; KAR2 and ERV2; KAR2 and EUG1; KAR2 and MPD1; KAR2 and MPD2; KAR2 and EPS1; KAR2 and PDI1; KAR2 and DER1; KAR2 and DER3; KAR2 and HRD3; KAR2 and UBC7; KAR2 and DOA4; KAR2 and HAC1; SIL1 and FKB2; SIL1 and SSA1; SIL1 and SSA2; SIL1 and SSA3; SIL1 and SSA4; SIL1 and SSE1; SIL1 and SSE2; SIL1 and SSB1; SIL1 and SSB2; SIL1 and ECM10; SIL1 and MDJ1; SIL1 and MDJ2; SIL1 and ERO1; SIL1 and ERV2; SIL1 and EUG1; SIL1 and MPD1; SIL1 and MPD2; SIL1 and EPS1; SIL1 and PDI1; SIL1 and DER1; SIL1 and DER3; SIL1 and HRD3; SIL1 and UBC7; SIL1 and DOA4; SIL1 and HAC1; FKB2 and SSA1; FKB2 and SSA2; FKB2 and SSA3; FKB2 and SSA4; FKB2 and SSE1; FKB2 and SSE2; FKB2 and SSB1; FKB2 and SSB2; FKB2 and ECM10; FKB2 and MDJ1; FKB2 and MDJ2; FKB2 and ERO1; FKB2 and ERV2; FKB2 and EUG1; FKB2 and MPD1; FKB2 and MPD2; FKB2 and EPS1; FKB2 and PDI1; FKB2 and DER1; FKB2 and DER3; FKB2 and HRD3; FKB2 and UBC7; FKB2 and DOA4; FKB2 and HAC1; SSA1 and SSA2; SSA1 and SSA3; SSA1 and SSA4; SSA1 and SSE1; SSA1 and SSE2; SSA1 and SSB1; SSA1 and SSB2; SSA1 and ECM10; SSA1 and MDJ1; SSA1 and MDJ2; SSA1 and ERO1; SSA1 and ERV2; SSA1 and EUG1; SSA1 and MPD1; SSA1 and MPD2; SSA1 and EPS1; SSA1 and PDI1; SSA1 and DER1; SSA1 and DER3; SSA1 and HRD3; SSA1 and UBC7; SSA1 and DOA4; SSA1 and HAC1; SSA2 and SSA3; SSA2 and SSA4; SSA2 and SSE1; SSA2 and SSE2; SSA2 and SSB1; SSA2 and SSB2; SSA2 and ECM10; SSA2 and MDJ1; SSA2 and MDJ2; SSA2 and ERO1; SSA2 and ERV2; SSA2 and EUG1; SSA2 and MPD1; SSA2 and MPD2; SSA2 and EPS1; SSA2 and PDI1; SSA2 and DER1; SSA2 and DER3; SSA2 and HRD3; SSA2 and UBC7; SSA2 and DOA4; SSA2 and HAC1; SSA3 and SSA4; SSA3 and SSE1; SSA3 and SSE2; SSA3 and SSB1; SSA3 and SSB2; SSA3 and ECM10; SSA3 and MDJ1; SSA3 and MDJ2; SSA3 and ERO1; SSA3 and ERV2; SSA3 and EUG1; SSA3 and MPD1; SSA3 and MPD2; SSA3 and EPS1; SSA3 and PDI1; SSA3 and DER1; SSA3 and DER3; SSA3 and HRD3; SSA3 and UBC7; SSA3 and DOA4; SSA3 and HAC1; SSA4 and SSE1; SSA4 and SSE2; SSA4 and SSB1; SSA4 and SSB2; SSA4 and ECM10; SSA4 and MDJ1; SSA4 and MDJ2; SSA4 and ERO1; SSA4 and ERV2; SSA4 and EUG1; SSA4 and MPD1; SSA4 and MPD2; SSA4 and EPS1; SSA4 and PDI1; SSA4 and DER1; SSA4 and DER3; SSA4 and HRD3; SSA4 and UBC7; SSA4 and DOA4; SSA4 and HAC1; SSE1 and SSE2; SSE1 and SSB1; SSE1 and SSB2; SSE1 and ECM10; SSE1 and MDJ1; SSE1 and MDJ2; SSE1 and ERO1; SSE1 and ERV2; SSE1 and EUG1; SSE1 and MPD1; SSE1 and MPD2; SSE1 and EPS1; SSE1 and PDI1; SSE1 and DER1; SSE1 and DER3; SSE1 and HRD3; SSE1 and UBC7; SSE1 and DOA4; SSE1 and HAC1; SSE2 and SSB1; SSE2 and SSB2; SSE2 and ECM10; SSE2 and MDJ1; SSE2 and MDJ2; SSE2 and ERO1; SSE2 and ERV2; SSE2 and EUG1; SSE2 and MPD1; SSE2 and MPD2; SSE2 and EPS1; SSE2 and PDI1; SSE2 and DER1; SSE2 and DER3; SSE2 and HRD3; SSE2 and UBC7; SSE2 and DOA4; SSE2 and HAC1; SSB1 and SSB2; SSB1 and ECM10; SSB1 and MDJ1; SSB1 and MDJ2; SSB1 and ERO1; SSB1 and ERV2; SSB1 and EUG1; SSB1 and MPD1; SSB1 and MPD2; SSB1 and EPS1; SSB1 and PDI1; SSB1 and DER1; SSB1 and DER3; SSB1 and HRD3; SSB1 and UBC7; SSB1 and DOA4; SSB1 and HAC1; SSB2 and ECM10; SSB2 and MDJ1; SSB2 and MDJ2; SSB2 and ERO1; SSB2 and ERV2; SSB2 and EUG1; SSB2 and MPD1; SSB2 and MPD2; SSB2 and EPS1; SSB2 and PDI1; SSB2 and DER1; SSB2 and DER3; SSB2 and HRD3; SSB2 and UBC7; SSB2 and DOA4; SSB2 and HAC1; ECM10 and MDJ1; ECM10 and MDJ2; ECM10 and ERO1; ECM10 and ERV2; ECM10 and EUG1; ECM10 and MPD1; ECM10 and MPD2; ECM10 and EPS1; ECM10 and PDI1; ECM10 and DER1; ECM10 and DER3; ECM10 and HRD3; ECM10 and UBC7; ECM10 and DOA4; ECM10 and HAC1; MDJ1 and MDJ2; MDJ1 and ERO1; MDJ1 and ERV2; MDJ1 and EUG1; MDJ1 and MPD1; MDJ1 and MPD2; MDJ1 and EPS1; MDJ1 and PDI1; MDJ1 and DER1; MDJ1 and DER3; MDJ1 and HRD3; MDJ1 and UBC7; MDJ1 and DOA4; MDJ1 and HAC1; MDJ2 and ERO1; MDJ2 and ERV2; MDJ2 and EUG1; MDJ2 and MPD1; MDJ2 and MPD2; MDJ2 and EPS1; MDJ2 and PDI1; MDJ2 and DER1; MDJ2 and DER3; MDJ2 and HRD3; MDJ2 and UBC7; MDJ2 and DOA4; MDJ2 and HAC1; ERO1 and ERV2; ERO1 and EUG1; ERO1 and MPD1; ERO1 and MPD2; ERO1 and EPS1; ERO1 and PDI1; ERO1 and DER1; ERO1 and DER3; ERO1 and HRD3; ERO1 and UBC7; ERO1 and DOA4; ERO1 and HAC1; ERV2 and EUG1; ERV2 and MPD1; ERV2 and MPD2; ERV2 and EPS1; ERV2 and PDI1; ERV2 and DER1; ERV2 and DER3; ERV2 and HRD3; ERV2 and UBC7; ERV2 and DOA4; ERV2 and HAC1; EUG1 and MPD1; EUG1 and MPD2; EUG1 and EPS1; EUG1 and PDI1; EUG1 and DER1; EUG1 and DER3; EUG1 and HRD3; EUG1 and UBC7; EUG1 and DOA4; EUG1 and HAC1; MPD1 and MPD2; MPD1 and EPS1; MPD1 and PDI1; MPD1 and DER1; MPD1 and DER3; MPD1 and HRD3; MPD1 and UBC7; MPD1 and DOA4; MPD1 and HAC1; MPD2 and EPS1; MPD2 and PDI1; MPD2 and DER1; MPD2 and DER3; MPD2 and HRD3; MPD2 and UBC7; MPD2 and DOA4; MPD2 and HAC1; EPS1 and PDI1; EPS1 and DER1; EPS1 and DER3; EPS1 and HRD3; EPS1 and UBC7; EPS1 and DOA4; EPS1 and HAC1; PDI1 and DER1; PDI1 and DER3; PDI1 and HRD3; PIM and UBC7; PDI1 and DOA4; PDI1 and HAC1; DER1 and DER3; DER1 and HRD3; DER1 and UBC7; DER1 and DOA4; DER1 and HAC1; DER3 and HRD3; DER3 and UBC7; DER3 and DOA4; DER3 and HAC1; HRD3 and UBC7; HRD3 and DOA4; HRD3 and HAC1; UBC7 and DOA4; UBC7 and HAC1; DOA4 and HAC1.

The skilled person will also appreciate that the present invention encompasses simultaneous over-expression of at least three helper proteins, and that the at least three helper proteins may or may not be taken from any combination of helper proteins derived from any of the above-defined groups.

For example, one of the following combinations of three helper proteins may or may not be simultaneously over-expressed, with or without the over-expression of one or more additional helper proteins:

JEM1 in combination with any one of the following combinations: LHS1 and SCJ1; LHS1 and KAR2; LHS1 and SIL1 LHS1 and FKB2; LHS1 and SSA1; LHS1 and SSA2; LHS1 and SSA3; LHS1 and SSA4; LHS1 and SSE1; LHS1 and SSE2; LHS1 and SSB1; LHS1 and SSB2; LHS1 and ECM10; LHS1 and MDJ1; LHS1 and MDJ2; LHS1 and ERO1; LHS1 and ERV2; LHS1 and EUG1; LHS1 and MPD1; LHS1 and MPD2; LHS1 and EPS1; LHS1 and PDI1; LHS1 and DER1; LHS1 and DER3; LHS1 and HRD3; LHS1 and UBC7; LHS1 and DOA4; LHS1 and HAC1; SCJ1 and KAR2; SCJ1 and SIL1; SCJ1 and FKB2; SCJ1 and SSA1; SCJ1 and SSA2; SCJ1 and SSA3; SCJ1 and SSA4; SCJ1 and SSE1; SCJ1 and SSE2; SCJ1 and SSB1; SCJ1 and SSB2; SCJ1 and ECM10; SCJ1 and MDJ1; SCJ1 and MDJ2; SCJ1 and ERO1; SCJ1 and ERV2; SCJ1 and EUG1; SCJ1 and MPD1; SCJ1 and MPD2; SCJ1 and EPS1; SCJ1 and PDI1; SCJ1 and DER1; SCJ1 and DER3; SCJ1 and HRD3; SCJ1 and UBC7; SCJ1 and DOA4; SCJ1 and HAC1; KAR2 and SIL1; KAR2 and FKB2; KAR2 and SSA1; KAR2 and SSA2; KAR2 and SSA3; KAR2 and SSA4; KAR2 and SSE1; KAR2 and SSE2; KAR2 and SSB1; KAR2 and SSB2; KAR2 and ECM10; KAR2 and MDJ1; KAR2 and MDJ2; KAR2 and ERO1; KAR2 and ERV2; KAR2 and EUG1; KAR2 and MPD1; KAR2 and MPD2; KAR2 and EPS1; KAR2 and PDI1; KAR2 and DER1; KAR2 and DER3; KAR2 and HRD3; KAR2 and UBC7; KAR2 and DOA4; KAR2 and HAC1; SIL1 and FKB2; SIL1 and SSA1; SIL1 and SSA2; SIL1 and SSA3; SIL1 and SSA4; SIL1 and SSE1; SIL1 and SSE2; SIL1 and SSB1; SIL1 and SSB2; SIL1 and ECM10; SIL1 and MDJ1; KU and MDJ2; SIL1 and ERO1; SIL1 and ERV2; SIL1 and EUG1; SIL1 and MPD1; SIL1 and MPD2; SIL1 and EPS1; SIL1 and PDI1; SIL1 and DER1; SIL1 and DER3; SIL1 and HRD3; SIL1 and UBC7; SIL1 and DOA4; SIL1 and HAC1; FKB2 and SSA1; FKB2 and SSA2; FKB2 and SSA3; FKB2 and SSA4; FKB2 and SSE1; FKB2 and SSE2; FKB2 and SSB1; FKB2 and SSB2; FKB2 and ECM10; FKB2 and MDJ1; FKB2 and MDJ2; FKB2 and ERO1; FKB2 and ERV2; FKB2 and EUG1; FKB2 and MPD1, FKB2 and MPD2; FKB2 and EPS1; FKB2 and PDI1; FKB2 and DER1; FKB2 and DER3; FKB2 and HRD3; FKB2 and UBC7; FKB2 and DOA4; FKB2 and HAC1; SSA1 and SSA2; SSA1 and SSA3; SSA1 and SSA4; SSA1 and SSE1; SSA1 and SSE2; SSA1 and SSB1; SSA1 and SSB2; SSA1 and ECM10; SSA1 and MDJ1; SSA1 and MDJ2; SSA1 and ERO1; SSA1 and ERV2; SSA1 and EUG1; SSA1 and MPD1; SSA1 and MPD2; SSA1 and EPS1; SSA1 and PDI1; SSA1 and DER1; SSA1 and DER3; SSA1 and HRD3; SSA1 and UBC7; SSA1 and DOA4; SSA1 and HAC1; SSA2 and SSA3; SSA2 and SSA4; SSA2 and SSE1; SSA2 and SSE2; SSA2 and SSB1; SSA2 and SSB2; SSA2 and ECM10; SSA2 and MDJ1; SSA2 and MDJ2; SSA2 and ERO1; SSA2 and ERV2; SSA2 and EUG1; SSA2 and MPD1; SSA2 and MPD2; SSA2 and EPS1; SSA2 and PDI1; SSA2 and DER1; SSA2 and DER3; SSA2 and HRD3; SSA2 and UBC7; SSA2 and DOA4; SSA2 and HAC1; SSA3 and SSA4; SSA3 and SSE1; SSA3 and SSE2; SSA3 and SSB1; SSA3 and SSB2; SSA3 and ECM10; SSA3 and MDJ1; SSA3 and MDJ2; SSA3 and ERO1; SSA3 and ERV2; SSA3 and EUG1; SSA3 and MPD1; SSA3 and MPD2; SSA3 and EPS1; SSA3 and PDI1; SSA3 and DER1; SSA3 and DER3; SSA3 and HRD3; SSA3 and UBC7; SSA3 and DOA4; SSA3 and HAC1; SSA4 and SSE1; SSA4 and SSE2; SSA4 and SSB1; SSA4 and SSB2; SSA4 and ECM10; SSA4 and MDJ1; SSA4 and MDJ2; SSA4 and ERO1; SSA4 and ERV2; SSA4 and EUG1; SSA4 and MPD1; SSA4 and MPD2; SSA4 and EPS1; SSA4 and PDI1; SSA4 and DER1; SSA4 and DER3; SSA4 and HRD3; SSA4 and UBC7; SSA4 and DOA4; SSA4 and HAC1; SSE1 and SSE2; SSE1 and SSB1; SSE1 and SSB2; SSE1 and ECM10; SSE1 and MDJ1; SSE1, and MDJ2; SSE1 and ERO1; SSE1 and ERV2; SSE1 and EUG1; SSE1 and MPD1; SSE1 and MPD2; SSE1 and EPS1; SSE1 and PDI1; SSE1 and DER1; SSE1 and DER3; SSE1 and HRD3; SSE1 and UBC7; SSE1 and DOA4; SSE1 and HAC1; SSE2 and SSB1; SSE2 and SSB2; SSE2 and ECM10; SSE2 and MDJ1; SSE2 and MDJ2; SSE2 and ERO1; SSE2 and ERV2; SSE2 and EUG1; SSE2 and MPD1; SSE2 and MPD2; SSE2 and EPS1; SSE2 and PDI1; SSE2 and DER1; SSE2 and DER3; SSE2 and HRD3; SSE2 and UBC7; SSE2 and DOA4; SSE2 and HAC1; SSB1 and SSB2; SSB1 and ECM10; SSB1 and MDJ1; SSB1 and MDJ2; SSB1 and ERO1; SSB1 and ERV2; SSB1 and EUG1; SSB1 and MPD1; SSB1 and MPD2; SSB1 and EPS1; SSB1 and PDI1; SSB1 and DER1; SSB1 and DER3; SSB1 and HRD3; SSB1 and UBC7; SSB1 and DOA4; SSB1 and HAC1; SSB2 and ECM10; SSB2 and MDJ1; SSB2 and MDJ2; SSB2 and ERO1; SSB2 and ERV2; SSB2 and EUG1; SSB2 and MPD1; SSB2 and MPD2; SSB2 and EPS1; SSB2 and PDI1; SSB2 and DER1; SSB2 and DER3; SSB2 and HRD3; SSB2 and UBC7; SSB2 and DOA4; SSB2 and HAC1; ECM10 and MDJ1; ECM10 and MDJ2; ECM10 and ERO1; ECM10 and ERV2; ECM10 and EUG1; ECM10 and MPD1; ECM10 and MPD2; ECM10 and EPS1; ECM10 and PDI1; ECM10 and DER1; ECM10 and DER3; ECM10 and HRD3; ECM10 and UBC7; ECM10 and DOA4; ECM10 and HAC1; MDJ1 and MDJ2; MDJ1 and ERO1; MDJ1 and ERV2; MDJ1 and EUG1; MDJ1 and MPD1; MDJ1 and MPD2; MDJ1 and EPS1; MDJ1 and PDI1; MDJ1 and DER1; MDJ1 and DER3; MDJ1 and HRD3; MDJ1 and UBC7; MDJ1 and DOA4; MDJ1 and HAC1; MDJ2 and ERO1; MDJ2 and ERV2; MDJ2 and EUG1; MDJ2 and MPD1; MDJ2 and MPD2; MDJ2 and EPS1; MDJ2 and PDI1; MDJ2 and DER1; MDJ2 and DER3; MDJ2 and HRD3; MDJ2 and UBC7; MDJ2 and DOA4; MDJ2 and HAC1; ERO1 and ERV2; ERO1 and EUG1; ERO1 and MPD1; ERO1 and MPD2; ERO1 and EPS1; ERO1 and PDI1; ERO1 and DER1; ERO1 and DER3; ERO1 and HRD3; ERO1 and UBC7; ERO1 and DOA4; ERO1 and HAC1; ERV2 and EUG1; ERV2 and MPD1; ERV2 and MPD2; ERV2 and EPS1; ERV2 and PDI1; ERV2 and DER1; ERV2 and DER3; ERV2 and HRD3; ERV2 and UBC7; ERV2 and DOA4; ERV2 and HAC1; EUG1 and MPD1; EUG1 and MPD2; EUG1 and EPS1; EUG1 and PDI1; EUG1 and DER1; EUG1 and DER3; EUG1 and HRD3; EUG1 and UBC7; EUG1 and DOA4; EUG1 and HAC1; MPD1 and MPD2; MPD1 and EPS1; MPD1 and PDI1; MPD1 and DER1; MPD1 and DER3; MPD1 and HRD3; MPD1 and UBC7; MPD1 and DOA4; MPD1 and HAC1; MPD2 and EPS1; MPD2 and PDI1; MPD2 and DER1; MPD2 and DER3; MPD2 and HRD3; MPD2 and UBC7; MPD2 and DOA4; MPD2 and HAC1; EPS1 and PDI1; EPS1 and DER1; EPS1 and DER3; EPS1 and HRD3; EPS1 and UBC7; EPS1 and DOA4; EPS1 and HAC1; PDI1 and DER1; PDI1 and DER3; PDI1 and HRD3; PDI1 and UBC7; PDI1 and DOA4; PDI1 and HAC1; DER1 and DER3; DER1 and HRD3; DER1 and UBC7; DER1 and DOA4; DER1 and HAC1; DER3 and HRD3; DER3 and UBC7; DER3 and DOA4; DER3 and HAC1; HRD3 and UBC7; HRD3 and DOA4; HRD3 and HAC1; UBC7 and DOA4; UBC7 and HAC1; or DOA4 and HAC1.

LHS1 in combination with any one of the following combinations: JEM1 and SCJ1; JEM1 and KAR2; JEM1 and SIL1; JEM1 and FKB2; JEM1 and SSA1; JEM1 and SSA2; JEM1 and SSA3; JEM1 and SSA4; JEM1 and SSE1; JEM1 and SSE2; JEM1 and SSB1; JEM1 and SSB2; JEM1 and ECM10; JEM1 and MDJ1; JEM1 and MDJ2; JEM1 and ERO1; JEM1 and ERV2; JEM1 and EUG1; JEM1 and MPD1; JEM1 and MPD2; JEM1 and EPS1; JEM1 and PDI1; JEM1 and DER1; JEM1 and DER3; JEM1 and HRD3; JEM1 and UBC7; JEM1 and DOA4; JEM1 and HAC1; SCJ1 and KAR2; SCJ1 and SIL1; SCJ1 and FKB2; SCJ1 and SSA1; SCJ1 and SSA2; SCJ1 and SSA3; SCJ1 and SSA4; SCJ1 and SSE1; SCJ1 and SSE2; SCJ1 and SSB1; SCJ1 and SSB2; SCJ1 and ECM10; SCJ1 and MDJ1; SCJ1 and MDJ2; SCJ1 and ERO1; SCJ1 and ERV2; SCJ1 and EUG1; SCJ1 and MPD1; SCJ1 and MPD2; SCJ1 and EPS1; SCJ1 and PDI1; SCJ1 and DER1; SCJ1 and DER3; SCJ1 and HRD3; SCJ1 and UBC7; SCJ1 and DOA4; SCJ1 and HAC1; KAR2 and SIL1; KAR2 and FKB2; KAR2 and SSA1; KAR2 and SSA2; KAR2 and SSA3; KAR2 and SSA4; KAR2 and SSE1; KAR2 and SSE2; KAR2 and SSB1; KAR2 and SSB2; KAR2 and ECM10; KAR2 and MDJ1; KAR2 and MDJ2; KAR2 and ERO1; KAR2 and ERV2; KAR2 and EUG1; KAR2 and MPD1; KAR2 and MPD2; KAR2 and EPS1; KAR2 and PDI1; KAR2 and DER1; KAR2 and DER3; KAR2 and HRD3; KAR2 and UBC7; KAR2 and DOA4; KAR2 and HAC1; SIL1 and FKB2; SIL1 and SSA1; SIL1 and SSA2; SIL1 and SSA3; SIL1 and SSA4; SIL1 and SSE1; SIL1 and SSE2; SIL1 and SSB1; SIL1 and SSB2; SIL1 and ECM10; SIL1 and MDJ1; SIL1 and MDJ2; SIL1 and ERO1; SIL1 and ERV2; SIL1 and EUG1; SIL1 and MPD1; SIL1 and MPD2; SIL1 and EPS1; SIL1 and PDI1; SIL1, and DER1; SIL1 and DER3; SIL1 and HRD3; SIL1 and UBC7; SIL1 and DOA4; SIL1 and HAC1; FKB2 and SSA1; FKB2 and SSA2; FKB2 and SSA3; FKB2 and SSA4; FKB2 and SSE1; FKB2 and SSE2; FKB2 and SSB1; FKB2 and SSB2; FKB2 and ECM10; FKB2 and MDJ1; FKB2 and MDJ2; FKB2 and ERO1; FKB2 and ERV2; FKB2 and EUG1; FKB2 and MPD1; FKB2 and MPD2; FKB2 and EPS1; FKB2 and PDI1; FKB2 and DER1; FKB2 and DER3; FKB2 and HRD3; FKB2 and UBC7; FKB2 and DOA4; FKB2 and HAC1; SSA1 and SSA2; SSA1 and SSA3; SSA1 and SSA4; SSA1 and SSE1; SSA1 and SSE2; SSA1 and SSB1; SSA1 and SSB2; SSA1 and ECM10; SSA1 and MDJ1; SSA1 and MDJ2; SSA1 and ERO1; SSA1 and ERV2; SSA1 and EUG1; SSA1 and MPD1; SSA1 and MPD2; SSA1 and EPS1; SSA1 and PDI1; SSA1 and DER1; SSA1 and DER3; SSA1 and HRD3; SSA1 and UBC7; SSA1 and DOA4; SSA1 and HAC1; SSA2 and SSA3; SSA2 and SSA4; SSA2 and SSE1; SSA2 and SSE2; SSA2 and SSB1; SSA2 and SSB2; SSA2 and ECM10; SSA2 and MDJ1; SSA2 and MDJ2; SSA2 and ERO1; SSA2 and ERV2; SSA2 and EUG1; SSA2 and MPD1; SSA2 and MPD2; SSA2 and EPS1; SSA2 and PDI1; SSA2 and DER1; SSA2 and DER3; SSA2 and HRD3; SSA2 and UBC7; SSA2 and DOA4; SSA2 and HAC1; SSA3 and SSA4; SSA3 and SSE1; SSA3 and SSE2; SSA3 and SSB1; SSA3 and SSB2; SSA3 and ECM10; SSA3 and MDJ1; SSA3 and MDJ2; SSA3 and ERO1; SSA3 and ERV2; SSA3 and EUG1; SSA3 and MPD1; SSA3 and MPD2; SSA3 and EPS1; SSA3 and PDI1; SSA3 and DER1; SSA3 and DER3; SSA3 and HRD3; SSA3 and UBC7; SSA3 and DOA4; SSA3 and HAC1; SSA4 and SSE1; SSA4 and SSE2; SSA4 and SSB1; SSA4 and SSB2; SSA4 and ECM10; SSA4 and MDJ1; SSA4 and MDJ2; SSA4 and ERO1; SSA4 and ERV2; SSA4 and EUG1; SSA4 and MPD1; SSA4 and MPD2; SSA4 and EPS1; SSA4 and PDI1; SSA4 and DER1; SSA4 and DER3; SSA4 and HRD3; SSA4 and UBC7; SSA4 and DOA4; SSA4 and HAC1; SSE1 and SSE2; SSE1 and SSB1; SSE1 and SSB2; SSE1 and ECM10; SSE1 and MDJ1; SSE1 and MDJ2; SSE1 and ERO1; SSE1 and ERV2; SSE1 and EUG1; SSE1 and MPD1; SSE1 and MPD2; SSE1 and EPS1; SSE1 and PDI1; SSE1 and DER1; SSE1 and DER3; SSE1 and HRD3; SSE1 and UBC7; SSE1 and DOA4; SSE1 and HAC1; SSE2 and SSB1; SSE2 and SSB2; SSE2 and ECM10; SSE2 and MDJ1; SSE2 and MDJ2; SSE2 and ERO1; SSE2 and ERV2; SSE2 and EUG1; SSE2 and MPD1; SSE2 and MPD2; SSE2 and EPS1; SSE2 and PDI1; SSE2 and DER1; SSE2 and DER3; SSE2 and HRD3; SSE2 and UBC7; SSE2 and DOA4; SSE2 and HAC1; SSB1 and SSB2; SSB1 and ECM10; SSB1 and MDJ1; SSB1 and MDJ2; SSB1 and ERO1; SSB1 and ERV2; SSB1 and EUG1; SSB1 and MPD1; SSB1 and MPD2; SSB1 and EPS1; SSB1 and PDI1; SSB1 and DER1; SSB1 and DER3; SSB1 and HRD3; SSB1 and UBC7; SSB1 and DOA4; SSB1 and HAC1; SSB2 and ECM10; SSB2 and MDJ1; SSB2 and MDJ2; SSB2 and ERO1; SSB2 and ERV2; SSB2 and EUG1; SSB2 and MPD1; SSB2 and MPD2; SSB2 and EPS1; SSB2 and PDI1; SSB2 and DER1; SSB2 and DER3; SSB2 and HRD3; SSB2 and UBC7; SSB2 and DOA4; SSB2 and HAC1; ECM10 and MDJ1; ECM10 and MDJ2; ECM10 and ERO1; ECM10 and ERV2; ECM10 and EUG1; ECM10 and MPD1; ECM10 and MPD2; ECM10 and EPS1; ECM10 and PDI1; ECM10 and DER1; ECM10 and DER3; ECM10 and HRD3; ECM10 and UBC7; ECM10 and DOA4; ECM10 and HAC1; MDJ1 and MDJ2; MDJ1 and ERO1; MDJ1 and ERV2; MDJ1 and EUG1; MDJ1 and MPD1; MDJ1 and MPD2; MDJ1 and EPS1; MDJ1 and PDI1; MDJ1 and DER1; MDJ1 and DER3; MDJ1 and HRD3; MDJ1 and UBC7; MDJ1 and DOA4; MDJ1 and HAC1; MDJ2 and ERO1; MDJ2 and ERV2; MDJ2 and EUG1; MDJ2 and MPD1; MDJ2 and MPD2; MDJ2 and EPS1; MDJ2 and PDI1; MDJ2 and DER1; MDJ2 and DER3; MDJ2 and HRD3; MDJ2 and UBC7; MDJ2 and DOA4; MDJ2 and HAC1; ERO1 and ERV2; ERO1 and EUG1; ERO1 and MPD1; ERO1 and MPD2; ERO1 and EPS1; ERO1 and PDI1; ERO1 and DER1; ERO1 and DER3; ERO1 and HRD3; ERO1 and UBC7; ERO1 and DOA4; ERO1 and HAC1; ERV2 and EUG1; ERV2 and MPD1; ERV2 and MPD2; ERV2 and EPS1; ERV2 and PDI1; ERV2 and DER1; ERV2 and DER3; ERV2 and HRD3; ERV2 and UBC7; ERV2 and DOA4; ERV2 and HAC1; EUG1 and MPD1; EUG1 and MPD2; EUG1 and EPS1; EUG1 and PDI1; EUG1 and DER1; EUG1 and DER3; EUG1 and HRD3; EUG1 and UBC7; EUG1 and DOA4; EUG1 and HAC1; MPD1 and MPD2; MPD1 and EPS1; MPD1 and PDI1; MPD1 and DER1; MPD1 and DER3; MPD1 and HRD3; MPD1 and UBC7; MPD1 and DOA4; MPD1 and HAC1; MPD2 and EPS1; MPD2 and PDI1; MPD2 and DER1; MPD2 and DER3; MPD2 and HRD3; MPD2 and UBC7; MPD2 and DOA4; MPD2 and HAC1; EPS1 and PDI1; EPS1 and DER1; EPS1 and DER3; EPS1 and HRD3; EPS1 and UBC7; EPS1 and DOA4; EPS1 and HAC1; PDI1 and DER1; PDI1 and DER3; PDI1 and HRD3; PDI1 and UBC7; PDI1 and DOA4; PDI1 and HAC1; DER1 and DER3; DER1 and HRD3; DER1 and UBC7; DER1 and DOA4; DER1 and HAC1; DER3 and HRD3; DER3 and UBC7; DER3 and DOA4; DER3 and HAC1; HRD3 and UBC7; HRD3 and DOA4; HRD3 and HAC1; UBC7 and DOA4; UBC7 and HAC1; or DOA4 and HAC1.

SCJ1 in combination with any one of the following combinations: JEM1 and LHS1; JEM1 and KAR2; JEM1 and SIL1; JEM1 and FKB2; JEM1 and SSA1; JEM1 and SSA2; JEM1 and SSA3; JEM1 and SSA4; JEM1 and SSE1; JEM1 and SSE2; JEM1 and SSB1; JEM1 and SSB2; JEM1 and ECM10; JEM1 and MDJ1; JEM1 and MDJ2; JEM1 and ERO1; JEM1 and ERV2; JEM1 and EUG1; JEM1 and MPD1; JEM1 and MPD2; JEM1 and EPS1; JEM1 and PDI1; JEM1 and DER1; JEM1 and DER3; JEM1 and HRD3; JEM1 and UBC7; JEM1 and DOA4; JEM1 and HAC1; LHS1 and KAR2; LHS1 and SIL1; LHS1 and FKB2; LHS1 and SSA1; LHS1 and SSA2; LHS1 and SSA3; LHS1 and SSA4; LHS1 and SSE1; LHS1 and SSE2; LHS1 and SSB1; LHS1 and SSB2; LHS1 and ECM10; LHS1 and MDJ1; LHS1 and MDJ2; LHS1 and ERO1; LHS1 and ERV2; LHS1 and EUG1; LHS1 and MPD1; LHS1 and MPD2; LHS1 and EPS1; LHS1, and PDI1; LHS1 and DER1; LHS1 and DER3; LHS1 and HRD3; LHS1 and UBC7; LHS1 and DOA4; LHS1 and HAC1; KAR2 and SIL1; KAR2 and FKB2; KAR2 and SSA1; KAR2 and SSA2; KAR2 and SSA3; KAR2 and SSA4; KAR2 and SSE1; KAR2 and SSE2; KAR2 and SSB1; KAR2 and SSB2; KAR2 and ECM10; KAR2 and MDJ1; KAR2 and MDJ2; KAR2 and ERO1; KAR2 and ERV2; KAR2 and EUG1; KAR2 and MPD1; KAR2 and MPD2; KAR2 and EPS1; KAR2 and PDI1; KAR2 and DER1; KAR2 and DER3; KAR2 and HRD3; KAR2 and UBC7; KAR2 and DOA4; KAR2 and HAC1; SIL1 and FKB2; SIL1 and SSA1; SIL1 and SSA2; SIL1 and SSA3; SIL1 and SSA4; SIL1 and SSE1; SIL1 and SSE2; SIL1 and SSB1; SIL1 and SSB2; SIL1 and ECM10; SIL1 and MDJ1; SIL1 and MDJ2; SIL1 and ERO1; SIL1 and ERV2; SIL1 and EUG1; SIL1 and MPD1; SIL1 and MPD2; SIL1 and EPS1; SIL1 and PDI1; SIL1 and DER1; SIL1 and DER3; HU and HRD3; SIL1 and UBC7; SIL1 and DOA4; SIL1 and HAC1; FKB2 and SSA1; FKB2 and SSA2; FKB2 and SSA3; FKB2 and SSA4; FKB2 and SSE1; FKB2 and SSE2; FKB2 and SSB1; FKB2 and SSB2; FKB2 and ECM10; FKB2 and MDJ1; FKB2 and MDJ2; FKB2 and ERO1; FKB2 and ERV2; FKB2 and EUG1; FKB2 and MPD1; FKB2 and MPD2; FKB2 and EPS1; FKB2 and PDI1; FKB2 and DER1; FKB2 and DER3; FKB2 and HRD3; FKB2 and UBC7; FKB2 and DOA4; FKB2 and HAC1; SSA1 and SSA2; SSA1 and SSA3; SSA1 and SSA4; SSA1 and SSE1; SSA1 and SSE2; SSA1 and SSB1; SSA1 and SSB2; SSA1 and ECM10; SSA1 and MDJ1; SSA1 and MDJ2; SSA1 and ERO1; SSA1 and ERV2; SSA1 and EUG1; SSA1 and MPD1; SSA1 and MPD2; SSA1 and EPS1; SSA1 and PDI1; SSA1 and DER1; SSA1 and DER3; SSA1 and HRD3; SSA1 and UBC7; SSA1 and DOA4; SSA1 and HAC1; SSA2 and SSA3; SSA2 and SSA4; SSA2 and SSE1; SSA2 and SSE2; SSA2 and SSB1; SSA2 and SSB2; SSA2 and ECM10; SSA2 and MDJ1; SSA2 and MDJ2; SSA2 and ERO1; SSA2 and ERV2; SSA2 and EUG1; SSA2 and MPD1; SSA2 and MPD2; SSA2 and EPS1; SSA2 and PDI1; SSA2 and DER1; SSA2 and DER3; SSA2 and HRD3; SSA2 and UBC7; SSA2 and DOA4; SSA2 and HAC1; SSA3 and SSA4; SSA3 and SSE1; SSA3 and SSE2; SSA3 and SSB1; SSA3 and SSB2; SSA3 and ECM10; SSA3 and MDJ1; SSA3 and MDJ2; SSA3 and ERO1; SSA3 and ERV2; SSA3 and EUG1; SSA3 and MPD1; SSA3 and MPD2; SSA3 and EPS1; SSA3 and PDI1; SSA3 and DER1; SSA3 and DER3; SSA3 and HRD3; SSA3 and UBC7; SSA3 and DOA4; SSA3 and HAC1; SSA4 and SSE1; SSA4 and SSE2; SSA4 and SSB1; SSA4 and SSB2; SSA4 and ECM10; SSA4 and MDJ1; SSA4 and MDJ2; SSA4 and ERO1; SSA4 and ERV2; SSA4 and EUG1; SSA4 and MPD1; SSA4 and MPD2; SSA4 and EPS1; SSA4 and PDI1; SSA4 and DER1; SSA4 and DER3; SSA4 and HRD3; SSA4 and UBC7; SSA4 and DOA4; SSA4 and HAC1; SSE1 and SSE2; SSE1 and SSB1; SSE1 and SSB2; SSE1 and ECM10; SSE1 and MDJ1; SSE1 and MDJ2; SSE1 and ERO1; SSE1 and ERV2; SSE1 and EUG1; SSE1 and MPD1; SSE1 and MPD2; SSE1 and EPS1; SSE1 and PDI1; SSE1 and DER1; SSE1 and DER3; SSE1 and HRD3; SSE1 and UBC7; SSE1 and DOA4; SSE1 and HAC1; SSE2 and SSB1; SSE2 and SSB2; SSE2 and ECM10; SSE2 and MDJ1; SSE2 and MDJ2; SSE2 and ERO1; SSE2 and ERV2; SSE2 and EUG1; SSE2 and MPD1; SSE2 and MPD2; SSE2 and EPS1; SSE2 and PDI1; SSE2 and DER1; SSE2 and DER3; SSE2 and HRD3; SSE2 and UBC7; SSE2 and DOA4; SSE2 and HAC1; SSB1 and SSB2; SSB1 and ECM10; SSB1 and MDJ1; SSB1 and MDJ2; SSB1 and ERO1; SSB1 and ERV2; SSB1 and EUG1; SSB1 and MPD1; SSB1 and MPD2; SSB1 and EPS1; SSB1 and PDI1; SSB1 and DER1; SSB1 and DER3; SSB1 and HRD3; SSB1 and UBC7; SSB1 and DOA4; SSB1 and HAC1; SSB2 and ECM10; SSB2 and MDJ1; SSB2 and MDJ2; SSB2 and ERO1; SSB2 and ERV2; SSB2 and EUG1; SSB2 and MPD1; SSB2 and MPD2; SSB2 and EPS1; SSB2 and PDI1; SSB2 and DER1; SSB2 and DER3; SSB2 and HRD3; SSB2 and UBC7; SSB2 and DOA4; SSB2 and HAC1; ECM10 and MDJ1; ECM10 and MDJ2; ECM10 and ERO1; ECM10 and ERV2; ECM10 and EUG1; ECM10 and MPD1; ECM10 and MPD2; ECM10 and EPS1; ECM10 and PDI1; ECM10 and DER1; ECM10 and DER3; ECM10 and HRD3; ECM10 and UBC7; ECM10 and DOA4; ECM10 and HAC1; MDJ1 and MDJ2; MDJ1 and ERO1; MDJ1 and ERV2; MDJ1 and EUG1; MDJ1 and MPD1; MDJ1 and MPD2; MDJ1 and EPS1; MDJ1 and PDI1; MDJ1 and DER1; MDJ1 and DER3; MDJ1 and HRD3; MDJ1 and UBC7; MDJ1 and DOA4; MDJ1 and HAC1; MDJ2 and ERO1; MDJ2 and ERV2; MDJ2 and EUG1; MDJ2 and MPD1; MDJ2 and MPD2; MDJ2 and EPS1; MDJ2 and PDI1; MDJ2 and DER1; MDJ2 and DER3; MDJ2 and HRD3; MDJ2 and UBC7; MDJ2 and DOA4; MDJ2 and HAC1; ERO1 and ERV2; ERO1 and EUG1; ERO1 and MPD1; ERO1 and MPD2; ERO1 and EPS1; ERO1 and PDI1; ERO1 and DER1; ERO1 and DER3; ERO1 and HRD3; ERO1 and UBC7; ERO1 and DOA4; ERO1 and HAC1; ERV2 and EUG1; ERV2 and MPD1; ERV2 and MPD2; ERV2 and EPS1; ERV2 and PDI1; ERV2 and DER1; ERV2 and DER3; ERV2 and HRD3; ERV2 and UBC7; ERV2 and DOA4; ERV2 and HAC1; EUG1 and MPD1; EUG1 and MPD2; EUG1 and EPS1; EUG1 and PDI1; EUG1 and DER1; EUG1 and DER3; EUG1 and HRD3; EUG1 and UBC7; EUG1 and DOA4; EUG1 and HAC1; MPD1 and MPD2; MPD1 and EPS1; MPD1 and PDI1; MPD1 and DER1; MPD1 and DER3; MPD1 and HRD3; MPD1 and UBC7; MPD1 and DOA4; MPD1 and HAC1; MPD2 and EPS1; MPD2 and PDI1; MPD2 and DER1; MPD2 and DER3; MPD2 and HRD3; MPD2 and UBC7; MPD2 and DOA4; MPD2 and HAC1; EPS1 and PDI1; EPS1 and DER1; EPS1 and DER3; EPS1 and HRD3; EPS1 and UBC7; EPS1 and DOA4; EPS1 and HAC1; PDI1 and DER1; PDI1 and DER3; PDI1 and HRD3; PDI1 and UBC7; PDI1 and DOA4; PDI1 and HAC1; DER1 and DER3; DER1 and HRD3; DER1 and UBC7; DER1 and DOA4; DER1 and HAC1; DER3 and HRD3; DER3 and UBC7; DER3 and DOA4; DER3 and HAC1; HRD3 and UBC7; HRD3 and DOA4; HRD3 and HAC1; UBC7 and DOA4; UBC7 and HAC1; or DOA4 and HAC1.

KAR2 in combination with any one of the following combinations: JEM1 and LHS1; JEM1 and SCJ1; JEM1 and SIL1; JEM1 and FKB2; JEM1 and SSA1; JEM1 and SSA2; JEM1 and SSA3; JEM1 and SSA4; JEM1 and SSE1; JEM1 and SSE2; JEM1 and SSB1; JEM1 and SSB2; JEM1 and ECM10; JEM1 and MDJ1; JEM1 and MDJ2; JEM1 and ERO1; JEM1 and ERV2; JEM1 and EUG1; JEM1 and MPD1; JEM1 and MPD2; JEM1 and EPS1; JEM1 and PDI1; JEM1 and DER1; JEM1 and DER3; JEM1 and HRD3; JEM1 and UBC7; JEM1 and DOA4; JEM1 and HAC1; LHS1 and SCJ1; LHS1 and SIL1 LHS1 and FKB2; LHS1 and SSA1; LHS1 and SSA2; LHS1 and SSA3; LHS1 and SSA4; LHS1 and SSE1; LHS1 and SSE2; LHS1 and SSB1; LHS1 and SSB2; LHS1 and ECM10; LHS1 and MDJ1; LHS1 and MDJ2; LHS1 and ERO1; LHS1 and ERV2; LHS1 and EUG1; LHS1 and MPD1; LHS1 and MPD2; LHS1 and EPS1; LHS1 and PDI1; LHS1 and DER1; LHS1 and DER3; LHS1 and HRD3; LHS1 and UBC7; LHS1 and DOA4; LHS1 and HAC1; SCJ1 and SIL1; SCJ1 and FKB2; SCJ1 and SSA1; SCJ1 and SSA2; SCJ1 and SSA3; SCJ1 and SSA4; SCJ1 and SSE1; SCJ1 and SSE2; SCJ1 and SSB1; SCJ1 and SSB2; SCJ1 and ECM10; SCJ1 and MDJ1; SCJ1 and MDJ2; SCJ1 and ERO1; SCJ1 and ERV2; SCJ1 and EUG1; SCJ1 and MPD1; SCJ1 and MPD2; SCJ1 and EPS1; SCJ1 and PDI1; SCJ1 and DER1; SCJ1 and DER3; SCJ1 and HRD3; SCJ1 and UBC7; SCJ1 and DOA4; SCJ1 and HAC1; SIL1 and FKB2; SIL1 and SSA1; SIL1 and SSA2; SIL1 and SSA3; SIL1 and SSA4; SIL1 and SSE1; SIL1 and SSE2; SIL1 and SSB1; SIL1 and SSB2; SIL1 and ECM10; SIL1 and MDJ1; SIL1 and MDJ2; SIL1 and ERO1; SIL1 and ERV2; SIL1 and EUG1; SIL1 and MPD1; SIL1 and MPD2; SIL1 and EPS1; SIL1 and PDI1; SIL1 and DER1; SIL1 and DER3; SIL1 and HRD3; SEM and UBC7; SIL1 and DOA4; SIL1 and HAC1; FKB2 and SSA1; FKB2 and SSA2; FKB2 and SSA3; FKB2 and SSA4; FKB2 and SSE1; FKB2 and SSE2; FKB2 and SSB1; FKB2 and SSB2; FKB2 and ECM10; FKB2 and MDJ1; FKB2 and MDJ2; FKB2 and ERO1; FKB2 and ERV2; FKB2 and EUG1; FKB2 and MPD1; FKB2 and MPD2; FKB2 and EPS1; FKB2 and PDI1; FKB2 and DER1; FKB2 and DER3; FKB2 and HRD3; FKB2 and UBC7; FKB2 and DOA4; FKB2 and HAC1; SSA1 and SSA2; SSA1 and SSA3; SSA1 and SSA4; SSA1 and SSE1; SSA1 and SSE2; SSA1 and SSB1; SSA1 and SSB2; SSA1 and ECM10; SSA1 and MDJ1; SSA1 and MDJ2; SSA1 and ERO1; SSA1 and ERV2; SSA1 and EUG1; SSA1 and MPD1; SSA1 and MPD2; SSA1 and EPS1; SSA1 and PDI1; SSA1 and DER1; SSA1 and DER3; SSA1 and HRD3; SSA1 and UBC7; SSA1 and DOA4; SSA1 and HAC1; SSA2 and SSA3; SSA2 and SSA4; SSA2 and SSE1; SSA2 and SSE2; SSA2 and SSB1; SSA2 and SSB2; SSA2 and ECM10; SSA2 and MDJ1; SSA2 and MDJ2; SSA2 and ERO1; SSA2 and ERV2; SSA2 and EUG1; SSA2 and MPD1; SSA2 and MPD2; SSA2 and EPS1; SSA2 and PDI1; SSA2 and DER1; SSA2 and DER3; SSA2 and HRD3; SSA2 and UBC7; SSA2 and DOA4; SSA2 and HAC1; SSA3 and SSA4; SSA3 and SSE1; SSA3 and SSE2; SSA3 and SSB1; SSA3 and SSB2; SSA3 and ECM10; SSA3 and MDJ1; SSA3 and MDJ2; SSA3 and ERO1; SSA3 and ERV2; SSA3 and EUG1; SSA3 and MPD1; SSA3 and MPD2; SSA3 and EPS1; SSA3 and PDI1; SSA3 and DER1; SSA3 and DER3; SSA3 and HRD3; SSA3 and UBC7; SSA3 and DOA4; SSA3 and HAC1; SSA4 and SSE1; SSA4 and SSE2; SSA4 and SSB1; SSA4 and SSB2; SSA4 and ECM10; SSA4 and MDJ1; SSA4 and MDJ2; SSA4 and ERO1; SSA4 and ERV2; SSA4 and EUG1; SSA4 and MPD1; SSA4 and MPD2; SSA4 and EPS1; SSA4 and PDI1; SSA4 and DER1; SSA4 and DER3; SSA4 and HRD3; SSA4 and UBC7; SSA4 and DOA4; SSA4 and HAC1; SSE1 and SSE2; SSE1 and SSB1; SSE1 and SSB2; SSE1 and ECM10; SSE1 and MDJ1; SSE1 and MDJ2; SSE1 and ERO1; SSE1 and ERV2; SSE1 and EUG1; SSE1 and MPD1; SSE1 and MPD2; SSE1 and EPS1; SSE1 and PDI1; SSE1 and DER1; SSE1 and DER3; SSE1 and HRD3; SSE1 and UBC7; SSE1 and DOA4; SSE1 and HAC1; SSE2 and SSB1; SSE2 and SSB2; SSE2 and ECM10; SSE2 and MDJ1; SSE2 and MDJ2; SSE2 and ERO1; SSE2 and ERV2; SSE2 and EUG1; SSE2 and MPD1; SSE2 and MPD2; SSE2 and EPS1; SSE2 and PDI1; SSE2 and DER1; SSE2 and DER3; SSE2 and HRD3; SSE2 and UBC7; SSE2 and DOA4; SSE2 and HAC1; SSB1 and SSB2; SSB1 and ECM10; SSB1 and MDJ1; SSB1 and MDJ2; SSB1 and ERO1; SSB1 and ERV2; SSB1 and EUG1; SSB1 and MPD1; SSB1 and MPD2; SSB1 and EPS1; SSB1 and PDI1; SSB1 and DER1; SSB1 and DER3; SSB1 and HRD3; SSB1 and UBC7; SSB1 and DOA4; SSB1 and HAC1; SSB2 and ECM10; SSB2 and MDJ1; SSB2 and MDJ2; SSB2 and ERO1; SSB2 and ERV2; SSB2 and EUG1; SSB2 and MPD1; SSB2 and MPD2; SSB2 and EPS1; SSB2 and PDI1; SSB2 and DER1; SSB2 and DER3; SSB2 and HRD3; SSB2 and UBC7; SSB2 and DOA4; SSB2 and HAC1; ECM10 and MDJ1; ECM10 and MDJ2; ECM10 and ERO1; ECM10 and ERV2; ECM10 and EUG1; ECM10 and MPD1; ECM10 and MPD2; ECM10 and EPS1; ECM10 and PDI1; ECM10 and DER1; ECM10 and DER3; ECM10 and HRD3; ECM10 and UBC7; ECM10 and DOA4; ECM10 and HAC1; MDJ1 and MDJ2; MDJ1 and ERO1; MDJ1 and ERV2; MDJ1 and EUG1; MDJ1 and MPD1; MDJ1 and MPD2; MDJ1 and EPS1; MDJ1 and PDI1; MDJ1 and DER1; MDJ1 and DER3; MDJ1 and HRD3; MDJ1 and UBC7; MDJ1 and DOA4; MDJ1 and HAC1; MDJ2 and ERO1; MDJ2 and ERV2; MDJ2 and EUG1; MDJ2 and MPD1; MDJ2 and MPD2; MDJ2 and EPS1; MDJ2 and PDI1; MDJ2 and DER1; MDJ2 and DER3; MDJ2 and HRD3; MDJ2 and UBC7; MDJ2 and DOA4; MDJ2 and HAC1; ERO1 and ERV2; ERO1 and EUG1; ERO1 and MPD1; ERO1 and MPD2; ERO1 and EPS1; ERO1 and PDI1; ERO1 and DER1; ERO1 and DER3; ERO1 and HRD3; ERO1 and UBC7; ERO1 and DOA4; ERO1 and HAC1; ERV2 and EUG1; ERV2 and MPD1; ERV2 and MPD2; ERV2 and EPS1; ERV2 and PDI1; ERV2 and DER1; ERV2 and DER3; ERV2 and HRD3; ERV2 and UBC7; ERV2 and DOA4; ERV2 and HAC1; EUG1 and MPD1; EUG1 and MPD2; EUG1 and EPS1; EUG1 and PDI1; EUG1 and DER1; EUG1 and DER3; EUG1 and HRD3; EUG1 and UBC7; EUG1 and DOA4; EUG1 and HAC1; MPD1 and MPD2; MPD1 and EPS1; MPD1 and PDI1; MPD1 and DER1; MPD1 and DER3; MPD1 and HRD3; MPD1 and UBC7; MPD1 and DOA4; MPD1 and HAC1; MPD2 and EPS1; MPD2 and PDI1; MPD2 and DER1; MPD2 and DER3; MPD2 and HRD3; MPD2 and UBC7; MPD2 and DOA4; MPD2 and HAC1; EPS1 and PDI1; EPS1 and DER1; EPS1 and DER3; EPS1 and HRD3; EPS1 and UBC7; EPS1 and DOA4; EPS1 and HAC1; PDI1 and DER1; PDI1 and DER3; PDI1 and HRD3; PDI1 and UBC7; PDI1 and DOA4; PDI1 and HAC1; DER1 and DER3; DER1 and HRD3; DER1 and UBC7; DER1 and DOA4; DER1 and HAC1; DER3 and HRD3; DER3 and UBC7; DER3 and DOA4; DER3 and HAC1; HRD3 and UBC7; HRD3 and DOA4; HRD3 and HAC1; UBC7 and DOA4; UBC7 and HAC1; or DOA4 and HAC1.

SIL1 in combination with any one of the following combinations: JEM1 and LHS1; JEM1 and SCJ1; JEM1 and KAR2; JEM1 and FKB2; JEM1 and SSA1; JEM1 and SSA2; JEM1 and SSA3; JEM1 and SSA4; JEM1 and SSE1; JEM1 and SSE2; JEM1 and SSB1; JEM1 and SSB2; JEM1 and ECM10; JEM1 and MDJ1; JEM1 and MDJ2; JEM1 and ERO1; JEM1 and ERV2; JEM1 and EUG1; JEM1 and MPD1; JEM1 and MPD2; JEM1 and EPS1; JEM1 and PDI1; JEM1 and DER1; JEM1 and DER3; JEM1 and HRD3; JEM1 and UBC7; JEM1 and DOA4; JEM1 and HAC1; LHS1 and SCJ1; LHS1 and KAR2; LHS1 and FKB2; LHS1 and SSA1; LHS1 and SSA2; LHS1 and SSA3; LHS1 and SSA4; LHS1 and SSE1; LHS1 and SSE2; LHS1 and SSB1; LHS1 and SSB2; LHS1 and ECM10; LHS1 and MDJ1; LHS1 and MDJ2; LHS1 and ERO1; LHS1 and ERV2; LHS1 and EUG1; LHS1 and MPD1; LHS1 and MPD2; LHS1 and EPS1; LHS1 and PDI1; LHS1 and DER1; LHS1 and DER3; LHS1 and HRD3; LHS1 and UBC7; LHS1 and DOA4; LHS1 and HAC1; SCJ1 and KAR2; SCJ1 and FKB2; SCJ1 and SSA1; SCJ1 and SSA2; SCJ1 and SSA3; SCJ1 and SSA4; SCJ1 and SSE1; SCJ1 and SSE2; SCJ1 and SSB1; SCJ1 and SSB2; SCJ1 and ECM10; SCJ1 and MDJ1; SCJ1 and MDJ2; SCJ1 and ERO1; SCJ1 and ERV2; SCJ1 and EUG1; SCJ1 and MPD1; SCJ1 and MPD2; SCJ1 and EPS1; SCJ1 and PDI1; SCJ1 and DER1; SCJ1 and DER3; SCJ1 and HRD3; SCJ1 and UBC7; SCJ1 and DOA4; SCJ1 and HAC1; KAR2 and FKB2; KAR2 and SSA1; KAR2 and SSA2; KAR2 and SSA3; KAR2 and SSA4; KAR2 and SSE1; KAR2 and SSE2; KAR2 and SSB1; KAR2 and SSB2; KAR2 and ECM10; KAR2 and MDJ1; KAR2 and MDJ2; KAR2 and ERO1; KAR2 and ERV2; KAR2 and EUG1; KAR2 and MPD1; KAR2 and MPD2; KAR2 and EPS1; KAR2 and PDI1; KAR2 and DER1; KAR2 and DER3; KAR2 and HRD3; KAR2 and UBC7; KAR2 and DOA4; KAR2 and HAC1; FKB2 and SSA1; FKB2 and SSA2; FKB2 and SSA3; FKB2 and SSA4; FKB2 and SSE1; FKB2 and SSE2; FKB2 and SSB1; FKB2 and SSB2; FKB2 and ECM10; FKB2 and MDJ1; FKB2 and MDJ2; FKB2 and ERO1; FKB2 and ERV2; FKB2 and EUG1; FKB2 and MPD1; FKB2 and MPD2; FKB2 and EPS1; FKB2 and PDI1; FKB2 and DER1; FKB2 and DER3; FKB2 and HRD3; FKB2 and UBC7; FKB2 and DOA4; FKB2 and HAC1; SSA1 and SSA2; SSA1 and SSA3; SSA1 and SSA4; SSA1 and SSE1; SSA1 and SSE2; SSA1 and SSB1; SSA1 and SSB2; SSA1 and ECM10; SSA1 and MDJ1; SSA1 and MDJ2; SSA1 and ERO1; SSA1 and ERV2; SSA1 and EUG1; SSA1 and MPD1; SSA1 and MPD2; SSA1 and EPS1; SSA1 and PDI1; SSA1 and DER1; SSA1 and DER3; SSA1 and HRD3; SSA1 and UBC7; SSA1 and DOA4; SSA1 and HAC1; SSA2 and SSA3; SSA2 and SSA4; SSA2 and SSE1; SSA2 and SSE2; SSA2 and SSB1; SSA2 and SSB2; SSA2 and ECM10; SSA2 and MDJ1; SSA2 and MDJ2; SSA2 and ERO1; SSA2 and ERV2; SSA2 and EUG1; SSA2 and MPD1; SSA2 and MPD2; SSA2 and EPS1; SSA2 and PDI1; SSA2 and DER1; SSA2 and DER3; SSA2 and HRD3; SSA2 and UBC7; SSA2 and DOA4; SSA2 and HAC1; SSA3 and SSA4; SSA3 and SSE1; SSA3 and SSE2; SSA3 and SSB1; SSA3 and SSB2; SSA3 and ECM10; SSA3 and MDJ1; SSA3 and MDJ2; SSA3 and ERO1; SSA3 and ERV2; SSA3 and EUG1; SSA3 and MPD1; SSA3 and MPD2; SSA3 and EPS1; SSA3 and PDI1; SSA3 and DER1; SSA3 and DER3; SSA3 and HRD3; SSA3 and UBC7; SSA3 and DOA4; SSA3 and HAC1; SSA4 and SSE1; SSA4 and SSE2; SSA4 and SSB1; SSA4 and SSB2; SSA4 and ECM10; SSA4 and MDJ1; SSA4 and MDJ2; SSA4 and ERO1; SSA4 and ERV2; SSA4 and EUG1; SSA4 and MPD1; SSA4 and MPD2; SSA4 and EPS1; SSA4 and PDI1; SSA4 and DER1; SSA4 and DER3; SSA4 and HRD3; SSA4 and UBC7; SSA4 and DOA4; SSA4 and HAC1; SSE1 and SSE2; SSE1 and SSB1; SSE1 and SSB2; SSE1 and ECM10; SSE1 and MDJ1; SSE1 and MDJ2; SSE1 and ERO1; SSE1 and ERV2; SSE1 and EUG1; SSE1 and MPD1; SSE1 and MPD2; SSE1 and EPS1; SSE1 and PDI1; SSE1 and DER1; SSE1 and DER3; SSE1 and HRD3; SSE1 and UBC7; SSE1 and DOA4; SSE1 and HAC1; SSE2 and SSB1; SSE2 and SSB2; SSE2 and ECM10; SSE2 and MDJ1; SSE2 and MDJ2; SSE2 and ERO1; SSE2 and ERV2; SSE2 and EUG1; SSE2 and MPD1; SSE2 and MPD2; SSE2 and EPS1; SSE2 and PDI1; SSE2 and DER1; SSE2 and DER3; SSE2 and HRD3; SSE2 and UBC7; SSE2 and DOA4; SSE2 and HAC1; SSB1 and SSB2; SSB1 and ECM10; SSB1 and MDJ1; SSB1 and MDJ2; SSB1 and ERO1; SSB1 and ERV2; SSB1 and EUG1; SSB1 and MPD1; SSB1 and MPD2; SSB1 and EPS1; SSB1 and PDI1; SSB1 and DER1; SSB1 and DER3; SSB1 and HRD3; SSB1 and UBC7; SSB1 and DOA4; SSB1 and HAC1; SSB2 and ECM10; SSB2 and MDJ1; SSB2 and MDJ2; SSB2 and ERO1; SSB2 and ERV2; SSB2 and EUG1; SSB2 and MPD1; SSB2 and MPD2; SSB2 and EPS1; SSB2 and PDI1; SSB2 and DER1; SSB2 and DER3; SSB2 and HRD3; SSB2 and UBC7; SSB2 and DOA4; SSB2 and HAC1; ECM10 and MDJ1; ECM10 and MDJ2; ECM10 and ERO1; ECM10 and ERV2; ECM10 and EUG1; ECM10 and MPD1; ECM10 and MPD2; ECM10 and EPS1; ECM10 and PDI1; ECM10 and DER1; ECM10 and DER3; ECM10 and HRD3; ECM10, and UBC7; ECM10 and DOA4; ECM10 and HAC1; MDJ1 and MDJ2; MDJ1 and ERO1; MDJ1 and ERV2; MDJ1 and EUG1; MDJ1 and MPD1; MDJ1 and MPD2; MDJ1 and EPS1; MDJ1 and PDI1; MDJ1 and DER1; MDJ1 and DER3; MDJ1 and HRD3; MDJ1 and UBC7; MDJ1 and DOA4; MDJ1 and HAC1; MDJ2 and ERO1; MDJ2 and ERV2; MDJ2 and EUG1; MDJ2 and MPD1; MDJ2 and MPD2; MDJ2 and EPS1; MDJ2 and PDI1; MDJ2 and DER1; MDJ2 and DER3; MDJ2 and HRD3; MDJ2 and UBC7; MDJ2 and DOA4; MDJ2 and HAC1; ERO1 and ERV2; ERO1 and EUG1; ERO1 and MPD1; ERO1 and MPD2; ERO1 and EPS1; ERO1 and PDI1; ERO1 and DER1; ERO1 and DER3; ERO1 and HRD3; ERO1 and UBC7; ERO1 and DOA4; ERO1 and HAC1; ERV2 and EUG1; ERV2 and MPD1; ERV2 and MPD2; ERV2 and EPS1; ERV2 and PDI1; ERV2 and DER1; ERV2 and DER3; ERV2 and HRD3; ERV2 and UBC7; ERV2 and DOA4; ERV2 and HAC1; EUG1 and MPD1; EUG1 and MPD2; EUG1 and EPS1; EUG1 and PDI1; EUG1 and DER1; EUG1 and DER3; EUG1 and HRD3; EUG1 and UBC7; EUG1 and DOA4; EUG1 and HAC1; MPD1 and MPD2; MPD1 and EPS1; MPD1 and PDI1; MPD1 and DER1; MPD1 and DER3; MPD1 and HRD3; MPD1 and UBC7; MPD1 and DOA4; MPD1 and HAC1; MPD2 and EPS1; MPD2 and PDI1; MPD2 and DER1; MPD2 and DER3; MPD2 and HRD3; MPD2 and UBC7; MPD2 and DOA4; MPD2 and HAC1; EPS1 and PDI1; EPS1 and DER1; EPS1 and DER3; EPS1 and HRD3; EPS1 and UBC7; EPS1 and DOA4; EPS1 and HAC1; PDI1 and DER1; PDI1 and DER3; PDI1 and HRD3; PDI1 and UBC7; PDI1 and DOA4; PDI1 and HAC1; DER1 and DER3; DER1 and HRD3; DER1 and UBC7; DER1 and DOA4; DER1 and HAC1; DER3 and HRD3; DER3 and UBC7; DER3 and DOA4; DER3 and HAC1; HRD3 and UBC7; HRD3 and DOA4; HRD3 and HAC1; UBC7 and DOA4; UBC7 and HAC1; or DOA4 and HAC1.

FKB2 in combination with any one of the following combinations: JEM1 and LHS1; JEM1 and SCJ1; JEM1 and KAR2; JEM1 and SIL1; JEM1 and SSA1; JEM1 and SSA2; JEM1 and SSA3; JEM1 and SSA4; JEM1 and SSE1; JEM1 and SSE2; JEM1 and SSB1; JEM1 and SSB2; JEM1 and ECM10; JEM1 and MDJ1; JEM1 and MDJ2; JEM1 and ERO1; JEM1 and ERV2; JEM1 and EUG1; JEM1 and MPD1; JEM1 and MPD2; JEM1 and EPS1; JEM1 and PDI1; JEM1 and DER1; JEM1 and DER3; JEM1 and HRD3; JEM1 and UBC7; JEM1 and DOA4; JEM1 and HAC1; LHS1 and SCJ1; LHS1 and KAR2; LHS1 and SIL1 LHS1 and SSA1; LHS1 and SSA2; LHS1 and SSA3; LHS1 and SSA4; LHS1 and SSE1; LHS1 and SSE2; LHS1 and SSB1; LHS1 and SSB2; LHS1 and ECM10; LHS1 and MDJ1; LHS1 and MDJ2; LHS1 and ERO1; LHS1 and ERV2; LHS1 and EUG1; LHS1 and MPD1; LHS1 and MPD2; LHS1 and EPS1; LHS1 and PDI1; LHS1 and DER1; LHS1 and DER3; LHS1 and HRD3; LHS1 and UBC7; LHS1 and DOA4; LHS1 and HAC1; SCJ1 and KAR2; SCJ1 and SIL1; SCJ1 and SSA1; SCJ1 and SSA2; SCJ1 and SSA3; SCJ1 and SSA4; SCJ1 and SSE1; SCJ1 and SSE2; SCJ1 and SSB1; SCJ1 and SSB2; SCJ1 and ECM10; SCJ1 and MDJ1; SCJ1 and MDJ2; SCJ1 and ERO1; SCJ1 and ERV2; SCJ1 and EUG1; SCJ1 and MPD1; SCJ1 and MPD2; SCJ1 and EPS1; SCJ1 and PDI1; SCJ1 and DER1; SCJ1 and DER3; SCJ1 and HRD3; SCJ1 and UBC7; SCJ1 and DOA4; SCJ1 and HAC1; KAR2 and SIL1; KAR2 and SSA1; KAR2 and SSA2; KAR2 and SSA3; KAR2 and SSA4; KAR2 and SSE1; KAR2 and SSE2; KAR2 and SSB1; KAR2 and SSB2; KAR2 and ECM10; KAR2 and MDJ1; KAR2 and MDJ2; KAR2 and ERO1; KAR2 and ERV2; KAR2 and EUG1; KAR2 and MPD1; KAR2 and MPD2; KAR2 and EPS1; KAR2 and PDI1; KAR2 and DER1; KAR2 and DER3; KAR2 and HRD3; KAR2 and UBC7; KAR2 and DOA4; KAR2 and HAC1; SIL1 and SSA1; SILT and SSA2; SIL1 and SSA3; SIL1 and SSA4; SIL1 and SSE1; SIL1 and SSE2; SIL1 and SSB1; SIL1 and SSB2; SIL1 and ECM10; SIL1 and MDJ1; SIL1 and MDJ2; SIL1 and ERO1; SIL1 and ERV2; SIL1 and EUG1; SIL1 and MPD1; SIL1 and MPD2; SIL1 and EPS1; SIL1 and PDI1; SIL1 and DER1; SIL1 and DER3; SIL1 and HRD3; SIL1 and UBC7; SIL1 and DOA4; SIL1 and HAC1; SSA1 and SSA2; SSA1 and SSA3; SSA1 and SSA4; SSA1 and SSE1; SSA1 and SSE2; SSA1 and SSB1; SSA1 and SSB2; SSA1 and ECM10; SSA1 and MDJ1; SSA1 and MDJ2; SSA1 and ERO1; SSA1 and ERV2; SSA1 and EUG1; SSA1 and MPD1; SSA1 and MPD2; SSA1 and EPS1; SSA1 and PDI1; SSA1 and DER1; SSA1 and DER3; SSA1 and HRD3; SSA1 and UBC7; SSA1 and DOA4; SSA1 and HAC1; SSA2 and SSA3; SSA2 and SSA4; SSA2 and SSE1; SSA2 and SSE2; SSA2 and SSB1; SSA2 and SSB2; SSA2 and ECM10; SSA2 and MDJ1; SSA2 and MDJ2; SSA2 and ERO1; SSA2 and ERV2; SSA2 and EUG1; SSA2 and MPD1; SSA2 and MPD2; SSA2 and EPS1; SSA2 and PDI1; SSA2 and DER1; SSA2 and DER3; SSA2 and HRD3; SSA2 and UBC7; SSA2 and DOA4; SSA2 and HAC1; SSA3 and SSA4; SSA3 and SSE1; SSA3 and SSE2; SSA3 and SSB1; SSA3 and SSB2; SSA3 and ECM10; SSA3 and MDJ1; SSA3 and MDJ2; SSA3 and ERO1; SSA3 and ERV2; SSA3 and EUG1; SSA3 and MPD1; SSA3 and MPD2; SSA3 and EPS1; SSA3 and PDI1; SSA3 and DER1; SSA3 and DER3; SSA3 and HRD3; SSA3 and UBC7; SSA3 and DOA4; SSA3 and HAC1; SSA4 and SSE1; SSA4 and SSE2; SSA4 and SSB1; SSA4 and SSB2; SSA4 and ECM10; SSA4 and MDJ1; SSA4 and MDJ2; SSA4 and ERO1; SSA4 and ERV2; SSA4 and EUG1; SSA4 and MPD1; SSA4 and MPD2; SSA4 and EPS1; SSA4 and PDI1; SSA4 and DER1; SSA4 and DER3; SSA4 and HRD3; SSA4 and UBC7; SSA4 and DOA4; SSA4 and HAC1; SSE1 and SSE2; SSE1 and SSB1; SSE1 and SSB2; SSE1 and ECM10; SSE1 and MDJ1; SSE1 and MDJ2; SSE1 and ERO1; SSE1 and ERV2; SSE1 and EUG1; SSE1 and MIDI; SSE1 and MPD2; SSE1 and EPS1; SSE1 and PDI1; SSE1 and DER1; SSE1 and DER3; SSE1 and HRD3; SSE1 and UBC7; SSE1 and DOA4; SSE1 and HAC1; SSE2 and SSB1; SSE2 and SSB2; SSE2 and ECM10; SSE2 and MDJ1; SSE2 and MDJ2; SSE2 and ERO1; SSE2 and ERV2; SSE2 and EUG1; SSE2 and MPD1; SSE2 and MPD2; SSE2 and EPS1; SSE2 and PDI1; SSE2 and DER1; SSE2 and DER3; SSE2 and HRD3; SSE2 and UBC7; SSE2 and DOA4; SSE2 and HAC1; SSB1 and SSB2; SSB1 and ECM10; SSB1 and MDJ1; SSB1 and MDJ2; SSB1 and ERO1; SSB1 and ERV2; SSB1 and EUG1; SSB1 and MPD1; SSB1 and MPD2; SSB1 and EPS1; SSB1 and PDI1; SSB1 and DER1; SSB1 and DER3; SSB1 and HRD3; SSB1 and UBC7; SSB1 and DOA4; SSB1 and HAC1; SSB2 and ECM10; SSB2 and MDJ1; SSB2 and MDJ2; SSB2 and ERO1; SSB2 and ERV2; SSB2 and EUG1; SSB2 and MPD1; SSB2 and MPD2; SSB2 and EPS1; SSB2 and PDI1; SSB2 and DER1; SSB2 and DER3; SSB2 and HRD3; SSB2 and UBC7; SSB2 and DOA4; SSB2 and HAC1; ECM10 and MDJ1; ECM10 and MDJ2; ECM10 and ERO1; ECM10 and ERV2; ECM10 and EUG1; ECM10 and MPD1; ECM10 and MPD2; ECM10 and EPS1; ECM10 and PDI1; ECM10 and DER1; ECM10 and DER3; ECM10 and HRD3; ECM10 and UBC7; ECM10 and DOA4; ECM10 and HAC1; MDJ1 and MDJ2; MDJ1 and ERO1; MDJ1 and ERV2; MDJ1 and EUG1; MDJ1 and MPD1; MDJ1 and MPD2; MDJ1 and EPS1; MDJ1 and PDI1; MDJ1 and DER1; MDJ1 and DER3; MDJ1 and HRD3; MDJ1 and UBC7; MDJ1 and DOA4; MDJ1 and HAC1; MDJ2 and ERO1; MDJ2 and ERV2; MDJ2 and EUG1; MDJ2 and MPD1; MDJ2 and MPD2; MDJ2 and EPS1; MDJ2 and PDI1; MDJ2- and DER1; MDJ2 and DER3; MDJ2 and HRD3; MDJ2 and UBC7; MDJ2 and DOA4; MDJ2 and HAC1; ERO1 and ERV2; ERO1 and EUG1; ERO1 and MPD1; ERO1 and MPD2; ERO1 and EPS1; ERO1 and PDI1; ERO1 and DER1; ERO1 and DER3; ERO1 and HRD3; ERO1 and UBC7; ERO1 and DOA4; ERO1 and HAC1; ERV2 and EUG1; ERV2 and MPD1; ERV2 and MPD2; ERV2 and EPS1; ERV2 and PDI1; ERV2 and DER1; ERV2 and DER3; ERV2 and HRD3; ERV2 and UBC7; ERV2 and DOA4; ERV2 and HAC1; EUG1 and MPD1; EUG1 and MPD2; EUG1 and EPS1; EUG1 and PDI1; EUG1 and DER1; EUG1 and DER3; EUG1 and HRD3; EUG1 and UBC7; EUG1 and DOA4; EUG1 and HAC1; MPD1 and MPD2; MPD1 and EPS1; MPD1 and PDI1; MPD1 and DER1; MPD1 and DER3; MPD1 and HRD3; MPD1 and UBC7; MPD1 and DOA4; MPD1 and HAC1; MPD2 and EPS1; MPD2 and PDI1; MPD2 and DER1; MPD2 and DER3; MPD2 and HRD3; MPD2 and UBC7; MPD2 and DOA4; MPD2 and HAC1; EPS1 and PDI1; EPS1 and DER1; EPS1 and DER3; EPS1 and HRD3; EPS1 and UBC7; EPS1 and DOA4; EPS1 and HAC1; PDI1 and DER1; PDI1 and DER3; PDI1 and HRD3; PDI1 and UBC7; PDI1 and DOA4; PDI1 and HAC1; DER1 and DER3; DER1 and HRD3; DER1 and UBC7; DER1 and DOA4; DER1 and HAC1; DER3 and HRD3; DER3 and UBC7; DER3 and DOA4; DER3 and HAC1; HRD3 and UBC7; HRD3 and DOA4; HRD3 and HAC1; UBC7 and DOA4; UBC7 and HAC1; or DOA4 and HAC1.

SSA1 in combination with any one of the following combinations: JEM1 and LHS1; JEM1 and SCJ1; JEM1 and KAR2; JEM1 and SIL1; JEM1 and FKB2; JEM1 and SSA2; JEM1 and SSA3; JEM1 and SSA4; JEM1 and SSE1; JEM1 and SSE2; JEM1 and SSB1; JEM1 and SSB2; JEM1 and ECM10; JEM1 and MDJ1; JEM1 and MDJ2; JEM1 and ERO1; JEM1 and ERV2; JEM1 and EUG1; JEM1 and MPD1; JEM1 and MPD2; JEM1 and EPS1; JEM1 and PDI1; JEM1 and DER1; JEM1 and DER3; JEM1 and HRD3; JEM1 and UBC7; JEM1 and DOA4; JEM1 and HAC1; LHS1 and SCJ1; LHS1 and KAR2; LHS1 and SIL1; LHS1 and FKB2; LHS1 and SSA2; LHS1 and SSA3; LHS1 and SSA4; LHS1 and SSE1; LHS1 and SSE2; LHS1 and SSB1; LHS1 and SSB2; LHS1 and ECM10; LHS1 and MDJ1; LHS1 and MDJ2; LHS1 and ERO1; LHS1 and ERV2; LHS1 and EUG1; LHS1 and MPD1; LHS1 and MPD2; LHS1 and EPS1; LHS1 and PDI1; LHS1 and DER1; LHS1 and DER3; LHS1 and HRD3; LHS1 and UBC7; LHS1 and DOA4; LHS1 and HAC1; SCJ1 and KAR2; SCJ1 and SIL1; SCJ1 and FKB2; SCJ1 and SSA2; SCJ1 and SSA3; SCJ1 and SSA4; SCJ1 and SSE1; SCJ1 and SSE2; SCJ1 and SSB1; SCJ1 and SSB2; SCJ1 and ECM10; SCJ1 and MDJ1; SCJ1 and MDJ2; SCJ1 and ERO1; SCJ1 and ERV2; SCJ1 and EUG1; SCJ1 and MPD1; SCJ1 and MPD2; SCJ1 and EPS1; SCJ1 and PDI1; SCJ1 and DER1; SCJ1 and DER3; SCJ1 and HRD3; SCJ1 and UBC7; SCJ1 and DOA4; SCJ1 and HAC1; KAR2 and SIL1; KAR2 and FKB2; KAR2 and SSA2; KAR2 and SSA3; KAR2 and SSA4; KAR2 and SSE1; KAR2 and SSE2; KAR2 and SSB1; KAR2 and SSB2; KAR2 and ECM10; KAR2 and MDJ1; KAR2 and MDJ2; KAR2 and ERO1; KAR2 and ERV2; KAR2 and EUG1; KAR2 and MPD1; KAR2 and MPD2; KAR2 and EPS1; KAR2 and PDI1; KAR2 and DER1; KAR2 and DER3; KAR2 and HRD3; KAR2 and UBC7; KAR2 and DOA4; KAR2 and HAC1; SIL1 and FKB2; SIL1 and SSA2; SIL1 and SSA3; SEM and SSA4; SIL1 and SSE1; SIL1 and SSE2; SIL1 and SSB1; SIL1 and SSB2; SIL1 and ECM10; SIL1 and MDJ1; SIL1 and MDJ2; SIL1 and ERO1; SIL1 and ERV2; SIL1 and EUG1; SIL1 and MPD1; SIL1 and MPD2; SIL1 and EPS1; SIL1 and PDI1; SIL1 and DER1; SIL1 and DER3; SIL1 and HRD3; SIL1 and UBC7; SIL1 and DOA4; SIL1 and HAC1; FKB2 and SSA2; FKB2 and SSA3; FKB2 and SSA4; FKB2 and SSE1; FKB2 and SSE2; FKB2 and SSB1; FKB2 and SSB2; FKB2 and ECM10; FKB2 and MDJ1; FKB2 and MDJ2; FKB2 and ERO1; FKB2 and ERV2; FKB2 and EUG1; FKB2 and MPD1; FKB2 and MPD2; FKB2 and EPS1; FKB2 and PDI1; FKB2 and DER1; FKB2 and DER3; FKB2 and HRD3; FKB2 and UBC7; FKB2 and DOA4; FKB2 and HAC1; SSA2 and SSA3; SSA2 and SSA4; SSA2 and SSE1; SSA2 and SSE2; SSA2 and SSB1; SSA2 and SSB2; SSA2 and ECM10; SSA2 and MDJ1; SSA2 and MDJ2; SSA2 and ERO1; SSA2 and ERV2; SSA2 and EUG1; SSA2 and MPD1; SSA2 and MPD2; SSA2 and EPS1; SSA2 and PDI1; SSA2 and DER1; SSA2 and DER3; SSA2 and HRD3; SSA2 and UBC7; SSA2 and DOA4; SSA2 and HAC1; SSA3 and SSA4; SSA3 and SSE1; SSA3 and SSE2; SSA3 and SSB1; SSA3 and SSB2; SSA3 and ECM10; SSA3 and MDJ1; SSA3 and MDJ2; SSA3 and ERO1; SSA3 and ERV2; SSA3 and EUG1; SSA3 and MPD1; SSA3 and MPD2; SSA3 and EPS1; SSA3 and PDI1; SSA3 and DER1; SSA3 and DER3; SSA3 and HRD3; SSA3 and UBC7; SSA3 DOA4; SSA3 and HAC1; SSA4 and SSE1; SSA4 acid SSE2; SSA4 and SSB1; SSA4 and SSB2; SSA4 and ECM10; SSA4 and MDJ1; SSA4 and MDJ2; SSA4 and ERO1; SSA4 and ERV2; SSA4 and EUG1; SSA4 and MPD1; SSA4 and MPD2; SSA4 and EPS1; SSA4 and PDI1; SSA4 and DER1; SSA4 and DER3; SSA4 and HRD3; SSA4 and UBC7; SSA4 and DOA4; SSA4 and HAC1; SSE1 and SSE2; SSE1 and SSB1; SSE1 and SSB2; SSE1 and ECM10; SSE1 and MDJ1; SSE1 and MDJ2; SSE1 and ERO1; SSE1 and ERV2; SSE1 and EUG1; SSE1 and MPD1; SSE1 and MPD2; SSE1 and EPS1; SSE1 and PDI1; SSE1 and DER1; SSE1 and DER3; SSE1 and HRD3; SSE1 and UBC7; SSE1 and DOA4; SSE1 and HAC1; SSE2 and SSB1; SSE2 and SSB2; SSE2 and ECM10; SSE2 and MDJ1; SSE2 and MDJ2; SSE2 and ERO1; SSE2 and ERV2; SSE2 and EUG1; SSE2 and MPD1; SSE2 and MPD2; SSE2 and EPS1; SSE2 and PDI1; SSE2 and DER1; SSE2 and DER3; SSE2 and HRD3; SSE2 and UBC7; SSE2 and DOA4; SSE2 and HAC1; SSB1 and SSB2; SSB1 and ECM10; SSB1 and MDJ1; SSB1 and MDJ2; SSB1 and ERO1; SSB1 and ERV2; SSB1 and EUG1; SSB1 and MPD1; SSB1 and MPD2; SSB1 and EPS1; SSB1 and PDI1; SSB1 and DER1; SSB1 and DER3; SSB1 and HRD3; SSB1 and UBC7; SSB1 and DOA4; SSB1 and HAC1; SSB2 and ECM10; SSB2 and MDJ1; SSB2 and MDJ2; SSB2 and ERO1; SSB2 and ERV2; SSB2 and EUG1; SSB2 and MPD1; SSB2 and MPD2; SSB2 and EPS1; SSB2 and PDI1; SSB2 and DER1; SSB2 and DER3; SSB2 and HRD3; SSB2 and UBC7; SSB2 and DOA4; SSB2 and HAC1; ECM10 and MDJ1; ECM10 and MDJ2; ECM10 and ERO1; ECM10 and ERV2; ECM10 and EUG1; ECM10 and MPD1; ECM10 and MPD2; ECM10 and EPS1; ECM10 and PDI1; ECM10 and DER1; ECM10 and DER3; ECM10 and HRD3; ECM10 and UBC7; ECM10 and DOA4; ECM10 and HAC1; MDJ1 and MDJ2; MDJ1 and ERO1; MDJ1 and ERV2; MDJ1 and EUG1; MDJ1 and MPD1; MDJ1 and MPD2; MDJ1 and EPS1; MDJ1 and PDI1; MDJ1 and DER1; MDJ1 and DER3; MDJ1 and HRD3; MDJ1 and UBC7; MDJ1 and DOA4; MDJ1 and HAC1; MDJ2 and ERO1; MDJ2 and ERV2; MDJ2 and EUG1; MDJ2 and MPD1; MDJ2 and MPD2; MDJ2 and EPS1; MDJ2 and PDI1; MDJ2 and DER1; MDJ2 and DER3; MDJ2 and HRD3; MDJ2 and UBC7; MDJ2 and DOA4; MDJ2 and HAC1; ERO1 and ERV2; ERO1 and EUG1; ERO1 and MPD1; ERO1 and MPD2; ERO1 and EPS1; ERO1 and PDI1; ERO1 and DER1; ERO1 and DER3; ERO1 and HRD3; ERO1 and UBC7; ERO1 and DOA4; ERO1 and HAC1; ERV2 and EUG1; ERV2 and MPD1; ERV2 and MPD2; ERV2 and EPS1; ERV2 and PDI1; ERV2 and DER1; ERV2 and DER3; ERV2 and HRD3; ERV2 and UBC7; ERV2 and DOA4; ERV2 and HAC1; EUG1 and MPD1; EUG1 and MPD2; EUG1 and EPS1; EUG1 and PDI1; EUG1 and DER1; EUG1 and DER3; EUG1 and HRD3; EUG1 and UBC7; EUG1 and DOA4; EUG1 and HAC1; MPD1 and MPD2; MPD1 and EPS1; MPD1 and PDI1; MPD1 and DER1; MPD1 and DER3; MPD1 and HRD3; MPD1 and UBC7; MPD1 and DOA4; MPD1 and HAC1; MPD2 and EPS1; MPD2 and PDI1; MPD2 and DER1; MPD2 and DER3; MPD2 and HRD3; MPD2 and UBC7; MPD2 and DOA4; MPD2 and HAC1; EPS1 and PDI1; EPS1 and DER1; EPS1 and DER3; EPS1 and HRD3; EPS1 and UBC7; EPS1 and DOA4; EPS1 and HAC1; PDI1 and DER1; PDI1 and DER3; PDI1 and HRD3; PDI1 and UBC7; PDI1 and DOA4; PDI1 and HAC1; DER1 and DER3; DER1 and HRD3; DER1 and UBC7; DER1 and DOA4; DER1 and HAC1; DER3 and HRD3; DER3 and UBC7; DER3 and DOA4; DER3 and HAC1; HRD3 and UBC7; HRD3 and DOA4; HRD3 and HAC1; UBC7 and DOA4; UBC7 and HAC1; or DOA4 and HAC1.

SSA2 in combination with any one of the following combinations: JEM1 and LHS1; JEM1 and SCJ1; JEM1 and KAR2; JEM1 and SIL1; JEM1 and FKB2; JEM1 and SSA1; JEM1 and SSA3; JEM1 and SSA4; JEM1 and SSE1; JEM1 and SSE2; JEM1 and SSB1; JEM1 and SSB2; JEM1 and ECM10; JEM1 and MDJ1; JEM1 and MDJ2; JEM1 and ERO1; JEM1 and ERV2; JEM1 and EUG1; JEM1 and MPD1; JEM1 and MPD2; JEM1 and EPS1; JEM1 and PDI1; JEM1 and DER1; JEM1 and DER3; JEM1 and HRD3; JEM1 and UBC7; JEM1 and DOA4; JEM1 and HAC1; LHS1 and SCJ1; LHS1 and KAR2; LHS1 and SIL1 LHS1 and FKB2; LHS1 and SSA1; LHS1 and SSA3; LHS1 and SSA4; LHS1 and SSE1; LHS1 and SSE2; LHS1 and SSB1; LHS1 and SSB2; LHS1 and ECM10; LHS1 and MDJ1; LHS1 and MDJ2; LHS1 and ERO1; LHS1 and ERV2; LHS1 and EUG1; LHS1 and MPD1; LHS1 and MPD2; LHS1 and EPS1; LHS1 and PDI1; LHS1 and DER1; LHS1 and DER3; LHS1 and HRD3; LHS1 and UBC7; LHS1 and DOA4; LHS1 and HAC1; SCJ1 and KAR2; SCJ1 and SIL1; SCJ1 and FKB2; SCJ1 and SSA1; SCJ1 and SSA3; SCJ1 and SSA4; SCJ1 and SSE1; SCJ1 and SSE2; SCJ1 and SSB1; SCJ1 and SSB2; SCJ1 and ECM10; SCJ1 and MDJ1; SCJ1 and MDJ2; SCJ1 and ERO1; SCJ1 and ERV2; SCJ1 and EUG1; SCJ1 and MPD1; SCJ1 and MPD2; SCJ1 and EPS1; SCJ1 and PDI1; SCJ1 and DER1; SCJ1 and DER3; SCJ1 and HRD3; SCJ1 and UBC7; SCJ1 and DOA4; SCJ1 and HAC1; KAR2 and SIL1; KAR2 and FKB2; KAR2 and SSA1; KAR2 and SSA3; KAR2 and SSA4; KAR2 and SSE1; KAR2 and SSE2; KAR2 and SSB1; KAR2 and SSB2; KAR2 and ECM10; KAR2 and MDJ1; KAR2 and MDJ2; KAR2 and ERO1; KAR2 and ERV2; KAR2 and EUG1; KAR2 and MPD1; KAR2 and MPD2; KAR2 and EPS1; KAR2 and PDI1; KAR2 and DER1; KAR2 and DER3; KAR2 and HRD3; KAR2 and UBC7; KAR2 and DOA4; KAR2 and HAC1; SIL1 and FKB2; SIL1 and SSA1; SIL1 and SSA3; SIL1 and SSA4; SIL1 and SSE1; SIL1 and SSE2; Sad and SSB1; SIL1 and SSB2; SIL1 and ECM10; SIL1 and MDJ1; SIL1 and MDJ2; SIL1 and ERO1; SIL1 and ERV2; SIL1 and EUG1; SIL1 and MPD1; SIL1 and MPD2; SIL1 and EPS1; SIL1 and PDI1; SIL1 and DER1; SIL1 and DER3; SIL1 and HRD3; SIL1 and UBC7; SIL1 and DOA4; SIL1 and HAC1; FKB2 and SSA1; FKB2 and SSA3; FKB2 and SSA4; FKB2 and SSE1; FKB2 and SSE2; FKB2 and SSB1; FKB2 and SSB2; FKB2 and ECM10; FKB2 and MDJ1; FKB2 and MDJ2; FKB2 and ERO1; FKB2 and ERV2; FKB2 and EUG1; FKB2 and MPD1; FKB2 and MPD2; FKB2 and EPS1; FKB2 and PDI1; FKB2 and DER1; FKB2 and DER3; FKB2 and HRD3; FKB2 and UBC7; FKB2 and DOA4; FKB2 and HAC1; SSA1 and SSA3; SSA1 and SSA4; SSA1 and SSE1; SSA1 and SSE2; SSA1 and SSB1; SSA1 and SSB2; SSA1 and ECM10; SSA1 and MDJ1; SSA1 and MDJ2; SSA1 and ERO1; SSA1 and ERV2; SSA1 and EUG1; SSA1 and MPD1; SSA1 and MPD2; SSA1 and EPS1; SSA1 and PDI1; SSA1 and DER1; SSA1 and DER3; SSA1 and HRD3; SSA1 and UBC7; SSA1 and DOA4; SSA1 and HAC1; SSA3 and SSA4; SSA3 and SSE1; SSA3 and SSE2; SSA3 and SSB1; SSA3 and SSB2; SSA3 and ECM10; SSA3 and MDJ1; SSA3 and MDJ2; SSA3 and ERO1; SSA3 and ERV2; SSA3 and EUG1; SSA3 and MPD1; SSA3 and MPD2; SSA3 and EPS1; SSA3 and PDI1; SSA3 and DER1; SSA3 and DER3; SSA3 and HRD3; SSA3 and UBC7; SSA3 and DOA4; SSA3 and HAC1; SSA4 and SSE1; SSA4 and SSE2; SSA4 and SSB1; SSA4 and SSB2; SSA4 and ECM10; SSA4 and MDJ1; SSA4 and MDJ2; SSA4 and ERO1; SSA4 and ERV2; SSA4 and EUG1; SSA4 and MPD1; SSA4 and MPD2; SSA4 and EPS1; SSA4 and PDI1; SSA4 and DER1; SSA4 and DER3; SSA4 and HRD3; SSA4 and UBC7; SSA4 and DOA4; SSA4 and HAC1; SSE1 and SSE2; SSE1 and SSB1; SSE1 and SSB2; SSE1 and ECM10; SSE1 and MDJ1; SSE1 and MDJ2; SSE1 and ERO1; SSE1 and ERV2; SSE1 and EUG1; SSE1 and MPD1; SSE1 and MPD2; SSE1 and EPS1; SSE1 and PDI1; SSE1 and DER1; SSE1 and DER3; SSE1 and HRD3; SSE1 and UBC7; SSE1 and DOA4; SSE1 and HAC1; SSE2 and SSB1; SSE2 and SSB2; SSE2 and ECM10; SSE2 and MDJ1; SSE2 and MDJ2; SSE2 and ERO1; SSE2 and ERV2; SSE2 and EUG1; SSE2 and MPD1; SSE2 and MPD2; SSE2 and EPS1; SSE2 and PDI1; SSE2 and DER1; SSE2 and DER3; SSE2 and HRD3; SSE2 and UBC7; SSE2 and DOA4; SSE2 and HAC1; SSB1 and SSB2; SSB1 and ECM10; SSB1 and MDJ1; SSB1 and MDJ2; SSB1 and ERO1; SSB1 and ERV2; SSB1 and EUG1; SSB1 and MPD1; SSB1 and MPD2; SSB1 and EPS1; SSB1 and PDI1; SSB1 and DER1; SSB1 and DER3; SSB1 and HRD3; SSB1 and UBC7; SSB1 and DOA4; SSB1 and HAC1; SSB2 and ECM10; SSB2 and MDJ1; SSB2 and MDJ2; SSB2 and ERO1; SSB2 and ERV2; SSB2 and EUG1; SSB2 and MPD1; SSB2 and MPD2; SSB2 and EPS1; SSB2 and PDI1; SSB2 and DER1; SSB2 and DER3; SSB2 and HRD3; SSB2 and UBC7; SSB2 and DOA4; SSB2 and HAC1; ECM10 and MDJ1; ECM10 and MDJ2; ECM10 and ERO1; ECM10 and ERV2; ECM10 and EUG1; ECM10 and MPD1; ECM10 and MPD2; ECM10 and EPS1; ECM10 and PDI1; ECM10 and DER1; ECM10 and DER3; ECM10 and HRD3; ECM10 and UBC7; ECM10 and DOA4; ECM10 and HAC1; MDJ1 and MDJ2; MDJ1 and ERO1; MDJ1 and ERV2; MDJ1 and EUG1; MDJ1 and MPD1; MDJ1 and MPD2; MDJ1 and EPS1; MDJ1 and PDI1; MDJ1 and DER1; MDJ1 and DER3; MDJ1 and HRD3; MDJ1 and UBC7; MDJ1 and DOA4; MDJ1 and HAC1; MDJ2 and ERO1; MDJ2 and ERV2; MDJ2 and EUG1; MDJ2 and MPD1; MDJ2 and MPD2; MDJ2 and EPS1; MDJ2 and PDI1; MDJ2 and DER1; MDJ2 and DER3; MDJ2 and HRD3; MDJ2 and UBC7; MDJ2 and DOA4; MDJ2 and HAC1; ERO1 and ERV2; ERO1 and EUG1; ERO1 and MPD1; ERO1 and MPD2; ERO1 and EPS1; ERO1 and PDI1; ERO1 and DER1; ERO1 and DER3; ERO1 and HRD3; ERO1 and UBC7; ERO1 and DOA4; ERO1 and HAC1; ERV2 and EUG1; ERV2 and MPD1; ERV2 and MPD2; ERV2 and EPS1; ERV2 and PDI1; ERV2 and DER1; ERV2 and DER3; ERV2 and HRD3; ERV2 and UBC7; ERV2 and DOA4; ERV2 and HAC1; EUG1 and MPD1; EUG1 and MPD2; EUG1 and EPS1; EUG1 and PDI1; EUG1 and DER1; EUG1 and DER3; EUG1 and HRD3; EUG1 and UBC7; EUG1 and DOA4; EUG1 and HAC1; MPD1 and MPD2; MPD1 and EPS1; MPD1 and PDI1; MPD1 and DER1; MPD1 and DER3; MPD1 and HRD3; MPD1 and UBC7; MPD1 and DOA4; MPD1 and HAC1; MPD2 and EPS1; MPD2 and PDI1; MPD2 and DER1; MPD2 and DER3; MPD2 and HRD3; MPD2 and UBC7; MPD2 and DOA4; MPD2 and HAC1; EPS1 and PDI1; EPS1 and DER1; EPS1 and DER3; EPS1 and HRD3; EPS1 and UBC7; EPS1 and DOA4; EPS1 and HAC1; PDI1 and DER1; PDI1 and DER3; PDI1 and HRD3; PDI1 and UBC7; PDI1 and DOA4; PDI1 and HAC1; DER1 and DER3; DER1 and HRD3; DER1 and UBC7; DER1 and DOA4; DER1 and HAC1; DER3 and HRD3; DER3 and UBC7; DER3 and DOA4; DER3 and HAC1; HRD3 and UBC7; HRD3 and DOA4; HRD3 and HAC1; UBC7 and DOA4; UBC7 and HAC1; or DOA4 and HAC1.

SSA3 in combination with any one of the following combinations: JEM1 and LHS1; JEM1 and SCJ1; JEM1 and KAR2; JEM1 and SIL1; JEM1 and FKB2; JEM1 and SSA1; JEM1 and SSA2; JEM1 and SSA4; JEM1 and SSE1; JEM1 and SSE2; JEM1 and SSB1; JEM1 and SSB2; JEM1 and ECM10; JEM1 and MDJ1; JEM1 and MDJ2; JEM1 and ERO1; JEM1 and ERV2; JEM1 and EUG1; JEM1 and MPD1; JEM1 and MPD2; JEM1 and EPS1; JEM1 and PDI1; JEM1 and DER1; JEM1 and DER3; JEM1 and HRD3; JEM1 and UBC7; JEM1 and DOA4; JEM1 and HAC1; LHS1 and SCJ1; LHS1 and KAR2; LHS1 and SIL1; LHS1 and FKB2; LHS1 and SSA1; LHS1 and SSA2; LHS1 and SSA4; LHS1 and SSE1; LHS1 and SSE2; LHS1 and SSB1; LHS1 and SSB2; LHS1 and ECM10; LHS1 and MDJ1; LHS1 and MDJ2; LHS1 and ERO1; LHS1 and ERV2; LHS1 and EUG1; LHS1 and MPD1; LHS1 and MPD2; LHS1 and EPS1; LHS1 and PDI1; LHS1 and DER1; LHS1 and DER3; LHS1 and HRD3; LHS1 and UBC7; LHS1 and DOA4; LHS1 and HAC1; SCJ1 and KAR2; SCJ1 and SIL1; SCH and FKB2; SCJ1 and SSA1; SCJ1 and SSA2; SCJ1 and SSA4; SCJ1 and SSE1; SCJ1 and SSE2; SCJ1 and SSB1; SCJ1 and SSB2; SCJ1 and ECM10; SCJ1 and MDJ1; SCJ1 and MDJ2; SCJ1 and ERO1; SCJ1 and ERV2; SCJ1 and EUG1; SCJ1 and MPD1; SCJ1 and MPD2; SCJ1 and EPS1; SCH and PDI1; SCJ1 and DER1; SCJ1 and DER3; SCJ1 and HRD3; SCJ1 and UBC7; SCJ1 and DOA4; SCJ1 and HAC1; KAR2 and SIL1; KAR2 and FKB2; KAR2 and SSA1; KAR2 and SSA2; KAR2 and SSA4; KAR2 and SSE1; KAR2 and SSE2; KAR2 and SSB1; KAR2 and SSB2; KAR2 and ECM10; KAR2 and MDJ1; KAR2 and MDJ2; KAR2 and ERO1; KAR2 and ERV2; KAR2 and EUG1; KAR2 and MPD1; KAR2 and MPD2; KAR2 and EPS1; KAR2 and PDI1; KAR2 and DER1; KAR2 and DER3; KAR2 and HRD3; KAR2 and UBC7; KAR2 and DOA4; KAR2 and HAC1; SIL1 and FKB2; SIL1 and SSA1; SIL1 and SSA2; SIL1 and SSA4; SIL1 and SSE1; SIL1 and SSE2; SIL1 and SSB1; SIL1 and SSB2; SIL1 and ECM10; SIL1 and MDJ1; SIL1 and MDJ2; SIL1 and ERO1; SILT and ERV2; SIL1 and EUG1; SIL1 and MPD1; SIL1 and MPD2; SIL1 and EPS1; SIL1 and PDI1; SIL1 and DER1; SIL1 and DER3; SIL1 and HRD3; SIL1 and UBC7; SIL1 and DOA4; SIL1 and HAC1; FKB2 and SSA1; FKB2 and SSA2; FKB2 and SSA4; FKB2 and SSE1; FKB2 and SSE2; FKB2 and SSB1; FKB2 and SSB2; FKB2 and ECM10; FKB2 and MDJ1; FKB2 and MDJ2; FKB2 and ERO1; FKB2 and ERV2; FKB2 and EUG1; FKB2 and MPD1; FKB2 and MPD2; FKB2 and EPS1; FKB2 and PDI1; FKB2 and DER1; FKB2 and DER3; FKB2 and HRD3; FKB2 and UBC7; FKB2 and DOA4; FKB2 and HAC1; SSA1 and SSA2; SSA1 and SSA4; SSA1 and SSE1; SSA1 and SSE2; SSA1 and SSB1; SSA1 and SSB2; SSA1 and ECM10; SSA1 and MDJ1; SSA1 and MDJ2; SSA1 and ERO1; SSA1 and ERV2; SSA1 and EUG1; SSA1 and MPD1; SSA1 and MPD2; SSA1 and EPS1; SSA1 and PDI1; SSA1 and DER1; SSA1 and DER3; SSA1 and HRD3; SSA1 and UBC7; SSA1 and DOA4; SSA1 and HAC1; SSA2 and SSA4; SSA2 and SSE1; SSA2 and SSE2; SSA2 and SSB1; SSA2 and SSB2; SSA2 and ECM10; SSA2 and MDJ1; SSA2 and MDJ2; SSA2 and ERO1; SSA2 and ERV2; SSA2 and EUG1; SSA2 and MPD1; SSA2 and MPD2; SSA2 and EPS1; SSA2 and PDI1; SSA2 and DER1; SSA2 and DER3; SSA2 and HRD3; SSA2 and UBC7; SSA2 and DOA4; SSA2 and HAC1; SSA4 and SSE1; SSA4 and SSE2; SSA4 and SSB1; SSA4 and SSB2; SSA4 and ECM10; SSA4 and MDJ1; SSA4 and MDJ2; SSA4 and ERO1; SSA4 and ERV2; SSA4 and EUG1; SSA4 and MPD1; SSA4 and MPD2; SSA4 and EPS1; SSA4 and PDI1; SSA4 and DER1; SSA4 and DER3; SSA4 and HRD3; SSA4 and UBC7; SSA4 and DOA4; SSA4 and HAC1; SSE1 and SSE2; SSE1 and SSB1; SSE1 and SSB2; SSE1 and ECM10; SSE1 and MDJ1; SSE1 and MDJ2; SSE1 and ERO1; SSE1 and ERV2; SSE1 and EUG1; SSE1 and MPD1; SSE1 and MPD2; SSE1 and EPS1; SSE1 and PDI1; SSE1 and DER1; SSE1 and DER3; SSE1 and HRD3; SSE1 and UBC7; SSE1 and DOA4; SSE1 and HAC1; SSE2 and SSB1; SSE2 and SSB2; SSE2 and ECM10; SSE2 and MDJ1; SSE2 and MDJ2; SSE2 and ERO1; SSE2 and ERV2; SSE2 and EUG1; SSE2 and MPD1; SSE2 and MPD2; SSE2 and EPS1; SSE2 and PDI1; SSE2 and DER1; SSE2 and DER3; SSE2 and HRD3; SSE2 and UBC7; SSE2 and DOA4; SSE2 and HAC1; SSB1 and SSB2; SSB1 and ECM10; SSB1 and MDJ1; SSB1 and MDJ2; SSB1 and ERO1; SSB1 and ERV2; SSB1 and EUG1; SSB1 and MPD1; SSB1 and MPD2; SSB1 and EPS1; SSB1 and PDI1; SSB1 and DER1; SSB1 and DER3; SSB1 and HRD3; SSB1 and UBC7; SSB1 and DOA4; SSB1 and HAC1; SSB2 and ECM10; SSB2 and MDJ1; SSB2 and MDJ2; SSB2 and ERO1; SSB2 and ERV2; SSB2 and EUG1; SSB2 and MPD1; SSB2 and MPD2; SSB2 and EPS1; SSB2 and PDI1; SSB2 and DER1; SSB2 and DER3; SSB2 and HRD3; SSB2 and UBC7; SSB2 and DOA4; SSB2 and HAC1; ECM10 and MDJ1; ECM10 and MDJ2; ECM10 and ERO1; ECM10 and ERV2; ECM10 and EUG1; ECM10 and MPD1; ECM10 and MPD2; ECM10 and EPS1; ECM10 and PDI1; ECM10 and DER1; ECM10 and DER3; ECM10 and HRD3; ECM10 and UBC7; ECM10 and DOA4; ECM10 and HAC1; MDJ1 and MDJ2; MDJ1 and ERO1; MDJ1 and ERV2; MDJ1 and EUG1; MDJ1 and MPD1; MDJ1 and MPD2; MDJ1 and EPS1; MDJ1 and PDI1; MDJ1 and DER1; MDJ1 and DER3; MDJ1 and HRD3; MDJ1 and UBC7; MDJ1 and DOA4; MDJ1 and HAC1; MDJ2 and ERO1; MDJ2 and ERV2; MDJ2 and EUG1; MDJ2 and MPD1; MDJ2 and MPD2; MDJ2 and EPS1; MDJ2 and PDI1; MDJ2 and DER1; MDJ2 and DER3; MDJ2 and HRD3; MDJ2 and UBC7; MDJ2 and DOA4; MDJ2 and HAC1; ERO1 and ERV2; ERO1 and EUG1; ERO1 and MPD1; ERO1 and MPD2; ERO1 and EPS1; ERO1 and PDI1; ERO1 and DER1; ERO1 and DER3; ERO1 and HRD3; ERO1 and UBC7; ERO1 and DOA4; ERO1 and HAC1; ERV2 and EUG1; ERV2 and MPD1; ERV2 and MPD2; ERV2 and EPS1; ERV2 and PDI1; ERV2 and DER1; ERV2 and DER3; ERV2 and HRD3; ERV2 and UBC7; ERV2 and DOA4; ERV2 and HAC1; EUG1 and MPD1; EUG1 and MPD2; EUG1 and EPS1; EUG1 and PDI1; EUG1 and DER1; EUG1 and DER3; EUG1 and HRD3; EUG1 and UBC7; EUG1 and DOA4; EUG1 and HAC1; MPD1 and MPD2; MPD1 and EPS1; MPD1 and PDI1; MPD1 and DER1; MPD1 and DER3; MPD1 and HRD3; MPD1 and UBC7; MPD1 and DOA4; MPD1 and HAC1; MPD2 and EPS1; MPD2 and PDI1; MPD2 and DER1; MPD2 and DER3; MPD2 and HRD3; MPD2 and UBC7; MPD2 and DOA4; MPD2 and HAC1; EPS1 and PDI1; EPS1 and DER1; EPS1 and DER3; EPS1 and HRD3; EPS1 and UBC7; EPS1 and DOA4; EPS1 and HAC1; PDI1 and DER1; PDI1 and DER3; PDI1 and HRD3; PDI1 and UBC7; PDI1 and DOA4; PDI1 and HAC1; DER1 and DER3; DER1 and HRD3; DER1 and UBC7; DER1 and DOA4; DER1 and HAC1; DER3 and HRD3; DER3 and UBC7; DER3 and DOA4; DER3 and HAC1; HRD3 and UBC7; HRD3 and DOA4; HRD3 and HAC1; UBC7 and DOA4; UBC7 and HAC1; or DOA4 and HAC1.

SSA4 in combination with any one of the following combinations: JEM1 and LHS1; JEM1 and SCJ1; JEM1 and KAR2; JEM1 and SIL1; JEM1 and FKB2; JEM1 and SSA1; JEM1 and SSA2; JEM1 and SSA3; JEM1 and SSE1; JEM1 and SSE2; JEM1 and SSB1; JEM1 and SSB2; JEM1 and ECM10; JEM1 and MDJ1; JEM1 and MDJ2; JEM1 and ERO1; JEM1 and ERV2; JEM1 and EUG1; JEM1 and MPD1; JEM1 and MPD2; JEM1 and EPS1; JEM1 and PDI1; JEM1 and DER1; JEM1 and DER3; JEM1 and HRD3; JEM1 and UBC7; JEM1 and DOA4; JEM1 and HAC1; LHS1 and SCJ1; LHS1 and KAR2; LHS1 and SIL1; LHS1 and FKB2; LHS1 and SSA1; LHS1 and SSA2; LHS1 and SSA3; LHS1 and SSE1; LHS1 and SSE2; LHS1 and SSB1; LHS1 and SSB2; LHS1 and ECM10; LHS1 and MDJ1; LHS1 and MDJ2; LHS1 and ERO1; LHS1 and ERV2; LHS1 and EUG1; LHS1 and MPD1; LHS1 and MPD2; LHS1 and EPS1; LHS1 and PDI1; LHS1 and DER1; LHS1 and DER3; LHS1 and HRD3; LHS1 and UBC7; LHS1 and DOA4; LHS1 and HAC1; SCJ1 and KAR2; SCJ1 and SIL1; SCJ1 and FKB2; SCJ1 and SSA1; SCJ1 and SSA2; SCJ1 and SSA3; SCJ1 and SSE1; SCJ1 and SSE2; SCJ1 and SSB1; SCJ1 and SSB2; SCJ1 and ECM10; SCJ1 and MDJ1; SCJ1 and MDJ2; SCJ1 and ERO1; SCJ1 and ERV2; SCJ1 and EUG1; SCJ1 and MPD1; SCJ1 and MPD2; SCJ1 and EPS1; SCJ1 and PDI1; SCJ1 and DER1; SCJ1 and DER3; SCJ1 and HRD3; SCJ1 and UBC7; SCJ1 and DOA4; SCJ1 and HAC1; KAR2 and SIL1; KAR2 and FKB2; KAR2 and SSA1; KAR2 and SSA2; KAR2 and SSA3; KAR2 and SSE1; KAR2 and SSE2; KAR2 and SSB1; KAR2 and SSB2; KAR2 and ECM10; KAR2 and MDJ1; KAR2 and MDJ2; KAR2 and ERO1; KAR2 and ERV2; KAR2 and EUG1; KAR2 and MPD1; KAR2 and MPD2; KAR2 and EPS1; KAR2 and PDI1; KAR2 and DER1; KAR2 and DER3; KAR2 and HRD3; KAR2 and UBC7; KAR2 and DOA4; KAR2 and HAC1; SIL1 and FKB2; SIL1 and SSA1; SIL1 and SSA2; SIL1 and SSA3; SIL1 and SSE1; SIL1 and SSE2; SIL1 and SSB1; SIL1 and SSB2; SIL1 and ECM10; SIL1 and MDJ1; SIL1 and MDJ2; SIL1 and ERO1; SIL1 and ERV2; SIL1 and EUG1; SIL1 and MPD1; SIL1 and MPD2; SIL1 and EPS1; SIL1 and PDI1; SIL1 and DER1; SIL1 and DER3; SIL1 and HRD3; SIL1 and UBC7; KU and DOA4; SIL1 and HAC1; FKB2 and SSA1; FKB2 and SSA2; FKB2 and SSA3; FKB2 and SSE1; FKB2 and SSE2; FKB2 and SSB1; FKB2 and SSB2; FKB2 and ECM10; FKB2 and MDJ1; FKB2 and MDJ2; FKB2 and ERO1; FKB2 and ERV2; FKB2 and EUG1; FKB2 and MPD1; FKB2 and MPD2; FKB2 and EPS1; FKB2 and PDI1; FKB2 and DER1; FKB2 and DER3; FKB2 and HRD3; FKB2 and UBC7; FKB2 and DOA4; FKB2 and HAC1; SSA1 and SSA2; SSA1 and SSA3; SSA1 and SSE1; SSA1 and SSE2; SSA1 and SSB1; SSA1 and SSB2; SSA1 and ECM10; SSA1 and MDJ1; SSA1 and MDJ2; SSA1 and ERO1; SSA1 and ERV2; SSA1 and EUG1; SSA1 and MPD1; SSA1 and MPD2; SSA1 and EPS1; SSA1 and PDI1; SSA1 and DER1; SSA1 and DER3; SSA1 and HRD3; SSA1 and UBC7; SSA1 and DOA4; SSA1 and HAC1; SSA2 and SSA3; SSA2 and SSE1; SSA2 and SSE2; SSA2 and SSB1; SSA2 and SSB2; SSA2 and ECM10; SSA2 and MDJ1; SSA2 and MDJ2; SSA2 and ERO1; SSA2 and ERV2; SSA2 and EUG1; SSA2 and MPD1; SSA2 and MPD2; SSA2 and EPS1; SSA2 and PDI1; SSA2 and DER1; SSA2 and DER3; SSA2 and HRD3; SSA2 and UBC7; SSA2 and DOA4; SSA2 and HAC1; SSA3 and SSE1; SSA3 and SSE2; SSA3 and SSB1; SSA3 and SSB2; SSA3 and ECM10; SSA3 and MDJ1; SSA3 and MDJ2; SSA3 and ERO1; SSA3 and ERV2; SSA3 and EUG1; SSA3 and MPD1; SSA3 and MPD2; SSA3 and EPS1; SSA3 and PDI1; SSA3 and DER1; SSA3 and DER3; SSA3 and HRD3; SSA3 and UBC7; SSA3 and DOA4; SSA3 and HAC1; SSE1 and SSE2; SSE1 and SSB1; SSE1 and SSB2; SSE1 and ECM10; SSE1 and MDJ1; SSE1 and MDJ2; SSE1 and ERO1; SSE1 and ERV2; SSE1 and EUG1; SSE1 and MPD1; SSE1 and MPD2; SSE1 and EPS1; SSE1 and PDI1; SSE1 and DER1; SSE1 and DER3; SSE1 and HRD3; SSE1 and UBC7; SSE1 and DOA4; SSE1 and HAC1; SSE2 and SSB1; SSE2 and SSB2; SSE2 and ECM10; SSE2 and MDJ1; SSE2 and MDJ2; SSE2 and ERO1; SSE2 and ERV2; SSE2 and EUG1; SSE2 and MPD1; SSE2 and MPD2; SSE2 and EPS1; SSE2 and PDI1; SSE2 and DER1; SSE2 and DER3; SSE2 and HRD3; SSE2 and UBC7; SSE2 and DOA4; SSE2 and HAC1; SSB1 and SSB2; SSB1 and ECM10; SSB1 and MDJ1; SSB1 and MDJ2; SSB1 and ERO1; SSB1 and ERV2; SSB1 and EUG1; SSB1 and MPD1; SSB1 and MPD2; SSB1 and EPS1; SSB1 and PDI1; SSB1 and DER1; SSB1 and DER3; SSB1 and HRD3; SSB1 and UBC7; SSB1 and DOA4; SSB1 and HAC1; SSB2 and ECM10; SSB2 and MDJ1; SSB2 and MDJ2; SSB2 and ERO1; SSB2 and ERV2; SSB2 and EUG1; SSB2 and MPD1; SSB2 and MPD2; SSB2 and EPS1; SSB2 and PDI1; SSB2 and DER1; SSB2 and DER3; SSB2 and HRD3; SSB2 and UBC7; SSB2 and DOA4; SSB2 and HAC1; ECM10 and MDJ1; ECM10 and MDJ2; ECM10 and ERO1; ECM10 and ERV2; ECM10 and EUG1; ECM10 and MPD1; ECM10 and MPD2; ECM10 and EPS1; ECM10 and PDI1; ECM10 and DER1; ECM10 and DER3; ECM10 and HRD3; ECM10 and UBC7; ECM10 and DOA4; ECM10 and HAC1; MDJ1 and MDJ2; MDJ1 and ERO1; MDJ1 and ERV2; MDJ1 and EUG1; MDJ1 and MPD1; MDJ1 and MPD2; MDJ1 and EPS1; MDJ1 and PDI1; MDJ1 and DER1; MDJ1 and DER3; MDJ1 and HRD3; MDJ1 and UBC7; MDJ1 and DOA4; MDJ1 and HAC1; MDJ2 and ERO1; MDJ2 and ERV2; MDJ2 and EUG1; MDJ2 and MPD1; MDJ2 and MPD2; MDJ2 and EPS1; MDJ2 and PDI1; MDJ2 and DER1; MDJ2 and DER3; MDJ2 and HRD3; MDJ2 and UBC7; MDJ2 and DOA4; MDJ2 and HAC1; ERO1 and ERV2; ERO1 and EUG1; ERO1 and MPD1; ERO1 and MPD2; ERO1 and EPS1; ERO1 and PDI1; ERO1 and DER1; ERO1 and DER3; ERO1 and HRD3; ERO1 and UBC7; ERO1 and DOA4; ERO1 and HAC1; ERV2 and EUG1; ERV2 and MPD1; ERV2 and MPD2; ERV2 and EPS1; ERV2 and PDI1; ERV2 and DER1; ERV2 and DER3; ERV2 and HRD3; ERV2 and UBC7; ERV2 and DOA4; ERV2 and HAC1; EUG1 and MPD1; EUG1 and MPD2; EUG1 and EPS1; EUG1 and PDI1; EUG1 and DER1; EUG1 and DER3; EUG1 and HRD3; EUG1 and UBC7; EUG1 and DOA4; EUG1 and HAC1; MPD1 and MPD2; MPD1 and EPS1; MPD1 and PDI1; MPD1 and DER1; MPD1 and DER3; MPD1 and HRD3; MPD1 and UBC7; MPD1 and DOA4; MPD1 and HAC1; MPD2 and EPS1; MPD2 and PDI1; MPD2 and DER1; MPD2 and DER3; MPD2 and HRD3; MPD2 and UBC7; MPD2 and DOA4; MPD2 and HAC1; EPS I and PDI1; EPS1 and DER1; EPS1 and DER3; EPS1 and HRD3; EPS1 and UBC7; EPS1 and DOA4; EPS1 and HAC1; PDI1 and DER1; PDI1 and DER3; PDI1 and HRD3; PDI1 and UBC7; PDI1 and DOA4; PDI1 and HAC1; DER1 and DER3; DER1 and HRD3; DER1 and UBC7; DER1 and DOA4; DER1 and HAC1; DER3 and HRD3; DER3 and UBC7; DER3 and DOA4; DER3 and HAC1; HRD3 and UBC7; HRD3 and DOA4; HRD3 and HAC1; UBC7 and DOA4; UBC7 and HAC1; or DOA4 and HAC1.

SSE1 in combination with any one of the following combinations: JEM1 and LHS1; JEM1 and SCJ1; JEM1 and KAR2; JEM1 and SIL1; JEM1 and FKB2; JEM1 and SSA1; JEM1 and SSA2; JEM1 and SSA3; JEM1 and SSA4; JEM1 and SSE2; JEM1 and SSB1; JEM1 and SSB2; JEM1 and ECM10; JEM1 and MDJ1; JEM1 and MDJ2; JEM1 and ERO1; JEM1 and ERV2; JEM1 and EUG1; JEM1 and MPD1; JEM1 and MPD2; JEM1 and EPS1; JEM1 and PDI1; JEM1 and DER1; JEM1 and DER3; JEM1 and HRD3; JEM1 and UBC7; JEM1 and DOA4; JEM1 and HAC1; LHS1 and SCJ1; LHS1 and KAR2; LHS1 and SIL1; LHS1 and FKB2; LHS1 and SSA1; LHS1 and SSA2; LHS1 and SSA3; LHS1 and SSA4; LHS1 and SSE2; LHS1 and SSB1; LHS1 and SSB2; LHS1 and ECM10; LHS1 and MDJ1; LHS1 and MDJ2; LHS1 and ERO1; LHS1 and ERV2; LHS1 and EUG1; LHS1 and MPD1; LHS1 and MPD2; LHS1 and EPS1; LHS1 and PDI1; LHS1 and DER1; LHS1 and DER3; LHS1 and HRD3; LHS1 and UBC7; LHS1 and DOA4; LHS1 and HAC1; SCJ1 and KAR2; SCJ1 and SIL1; SCJ1 and FKB2; SCJ1 and SSA1; SCJ1 and SSA2; SCJ1 and SSA3; SCJ1 and SSA4; SCJ1 and SSE2; SCJ1 and SSB1; SCJ1 and SSB2; SCJ1 and ECM10; SCJ1 and MDJ1; SCJ1 and MDJ2; SCJ1 and ERO1; SCJ1 and ERV2; SCJ1 and EUG1; SCJ1 and MPD1; SCJ1 and MPD2; SCJ1 and EPS1; SCJ1 and PDI1; SCJ1 and DER1; SCJ1 and DER3; SCJ1 and HRD3; SCJ1 and UBC7; SCJ1 and DOA4; SCJ1 and HAC1; KAR2 and SIL1; KAR2 and FKB2; KAR2 and SSA1; KAR2 and SSA2; KAR2 and SSA3; KAR2 and SSA4; KAR2 and SSE2; KAR2 and SSB1; KAR2 and SSB2; KAR2 and ECM10; KAR2 and MDJ1; KAR2 and MDJ2; KAR2 and ERO1; KAR2 and ERV2; KAR2 and EUG1; KAR2 and MPD1; KAR2 and MPD2; KAR2 and EPS1; KAR2 and PDI1; KAR2 and DER1; KAR2 and DER3; KAR2 and HRD3; KAR2 and UBC7; KAR2 and DOA4; KAR2 and HAC1; SIL1 and FKB2; SIL1 and SSA1; SIL1 and SSA2; SIL1 and SSA3; SIL1 and SSA4; SIL1 and SSE2; SIL1 and SSB1; SIL1 and SSB2; SIL1 and ECM10; SIL1 and MDJ1; SIL1 and MDJ2; SIL1 and ERO1; SIL1 and ERV2; SIL1 and EUG1; SIL1 and MPD1; SIL1 and MPD2; SIL1 and EPS1; SIL1 and PDI1; SIL1 and DER1; SIL1 and DER3; SIL1 and HRD3; SIL1 and UBC7; SIL1 and DOA4; SIL1 and HAC1; FKB2 and SSA1; FKB2 and SSA2; FKB2 and SSA3; FKB2 and SSA4; FKB2 and SSE2; FKB2 and SSB1; FKB2 and SSB2; FKB2 and ECM10; FKB2 and MDJ1; FKB2 and MDJ2; FKB2 and ERO1; FKB2 and ERV2; FKB2 and EUG1; FKB2 and MPD1; FKB2 and MPD2; FKB2 and EPS1; FKB2 and PDI1; FKB2 and DER1; FKB2 and DER3; FKB2 and HRD3; FKB2 and UBC7; FKB2 and DOA4; FKB2 and HAC1; SSA1 and SSA2; SSA1 and SSA3; SSA1 and SSA4; SSA1 and SSE2; SSA1 and SSB1; SSA1 and SSB2; SSA1 and ECM10; SSA1 and MDJ1; SSA1 and MDJ2; SSA1 and ERO1; SSA1 and ERV2; SSA1 and EUG1; SSA1 and MPD1; SSA1 and MPD2; SSA1 and EPS1; SSA1 and PDI1; SSA1 and DER1; SSA1 and DER3; SSA1 and HRD3; SSA1 and UBC7; SSA1 and DOA4; SSA1 and HAC1; SSA2 and SSA3; SSA2 and SSA4; SSA2 and SSE2; SSA2 and SSB1; SSA2 and SSB2; SSA2 and ECM10; SSA2 and MDJ1; SSA2 and MDJ2; SSA2 and ERO1; SSA2 and ERV2; SSA2 and EUG1; SSA2 and MPD1; SSA2 and MPD2; SSA2 and EPS1; SSA2 and PDI1; SSA2 and DER1; SSA2 and DER3; SSA2 and HRD3; SSA2 and UBC7; SSA2 and DOA4; SSA2 and HAC1; SSA3 and SSA4; SSA3 and SSE2; SSA3 and SSB1; SSA3 and SSB2; SSA3 and ECM10; SSA3 and MDJ1; SSA3 and MDJ2; SSA3 and ERO1; SSA3 and ERV2; SSA3 and EUG1; SSA3 and MPD1; SSA3 and MPD2; SSA3 and EPS1; SSA3 and PDI1; SSA3 and DER1; SSA3 and DER3; SSA3 and HRD3; SSA3 and UBC7; SSA3 and DOA4; SSA3 and HAC1; SSA4 and SSE2; SSA4 and SSB1; SSA4 and SSB2; SSA4 and ECM10; SSA4 and MDJ1; SSA4 and MDJ2; SSA4 and ERO1; SSA4 and ERV2; SSA4 and EUG1; SSA4 and MPD1; SSA4 and MPD2; SSA4 and EPS1; SSA4 and PDI1; SSA4 and DER1; SSA4 and DER3; SSA4 and HRD3; SSA4 and UBC7; SSA4 and DOA4; SSA4 and HAC1; SSE2 and SSB1; SSE2 and SSB2; SSE2 and ECM10; SSE2 and MDJ1; SSE2 and MDJ2; SSE2 and ERO1; SSE2 and ERV2; SSE2 and EUG1; SSE2 and MPD1; SSE2 and MPD2; SSE2 and EPS1; SSE2 and PDI1; SSE2 and DER1; SSE2 and DER3; SSE2 and HRD3; SSE2 and UBC7; SSE2 and DOA4; SSE2 and HAC1; SSB1 and SSB2; SSB1 and ECM10; SSB1 and MDJ1; SSB1 and MDJ2; SSB1 and ERO1; SSB1 and ERV2; SSB1 and EUG1; SSB1 and MPD1; SSB1 and MPD2; SSB1 and EPS1; SSB1 and PDI1; SSB1 and DER1; SSB1 and DER3; SSB1 and HRD3; SSB1 and UBC7; SSB1 and DOA4; SSB1 and HAC1; SSB2 and ECM10; SSB2 and MDJ1; SSB2 and MDJ2; SSB2 and ERO1; SSB2 and ERV2; SSB2 and EUG1; SSB2 and MPD1; SSB2 and MPD2; SSB2 and EPS1; SSB2 and PDI1; SSB2 and DER1; SSB2 and DER3; SSB2 and HRD3; SSB2 and UBC7; SSB2 and DOA4; SSB2 and HAC1; ECM10 and MDJ1; ECM10 and MDJ2; ECM10 and ERO1; ECM10 and ERV2; ECM10 and EUG1; ECM10 and MPD1; ECM10 and MPD2; ECM10 and EPS1; ECM10 and PDI1; ECM10 and DER1; ECM10 and DER3; ECM10 and HRD3; ECM10 and UBC7; ECM10 and DOA4; ECM10 and HAC1; MDJ1 and MDJ2; MDJ1 and ERO1; MDJ1 and ERV2; MDJ1 and EUG1; MDJ1 and MPD1; MDJ1 and MPD2; MDJ1 and EPS1; MDJ1 and PDI1; MDJ1 and DER1; MDJ1 and DER3; MDJ1 and HRD3; MDJ1 and UBC7; MDJ1 and DOA4; MDJ1 and HAC1; MDJ2 and ERO1; MDJ2 and ERV2; MDJ2 and EUG1; MDJ2 and MPD1; MDJ2 and MPD2; MDJ2 and EPS1; MDJ2 and PDI1; MDJ2 and DER1; MDJ2 and DER3; MDJ2 and HRD3; MDJ2 and UBC7; MDJ2 and DOA4; MDJ2 and HAC1; ERO1 and ERV2; ERO1 and EUG1; ERO1 and MPD1; ERO1 and MPD2; ERO1 and EPS1; ERO1 and PDI1; ERO1 and DER1; ERO1 and DER3; ERO1 and HRD3; ERO1 and UBC7; ERO1 and DOA4; ERO1 and HAC1; ERV2 and EUG1; ERV2 and MPD1; ERV2 and MPD2; ERV2 and EPS1; ERV2 and PDI1; ERV2 and DER1; ERV2 and DER3; ERV2 and HRD3; ERV2 and UBC7; ERV2 and DOA4; ERV2 and HAC1; EUG1 and MPD1; EUG1 and MPD2; EUG1 and EPS1; EUG1 and PDI1; EUG1 and DER1; EUG1 and DER3; EUG1 and HRD3; EUG1 and UBC7; EUG1 and DOA4; EUG1 and HAC1; MPD1 and MPD2; MPD1 and EPS1; MPD1 and PDI1; MPD1 and DER1; MPD1 and DER3; MPD1 and HRD3; MPD1 and UBC7; MPD1 and DOA4; MPD1 and HAC1; MPD2 and EPS1; MPD2 and PDI1; MPD2 and DER1; MPD2 and DER3; MPD2 and HRD3; MPD2 and UBC7; MPD2 and DOA4; MPD2 and HAC1; EPS1 and PDI1; EPS1 and DER1; EPS-1 and DER3; EPS1 and HRD3; EPS1 and UBC7; EPS1 and DOA4; EPS1 and HAC1; PDI1 and DER1; PDI1 and DER3; PDI1 and HRD3; PDI1 and UBC7; PDI1 and DOA4; PDI1 and HAC1; DER1 and DER3; DER1 and HRD3; DER1 and UBC7; DER1 and DOA4; DER1 and HAC1; DER3 and HRD3; DER3 and UBC7; DER3 and DOA4; DER3 and HAC1; HRD3 and UBC7; HRD3 and DOA4; HRD3 and HAC1; UBC7 and DOA4; UBC7 and HAC1; or DOA4 and HAC1.

SSE2 in combination with any one of the following combinations: JEM1 and LHS1; JEM1 and SCJ1; JEM1 and KAR2; JEM1 and SIL1; JEM1 and FKB2; JEM1 and SSA1; JEM1 and SSA2; JEM1 and SSA3; JEM1 and SSA4; JEM1 and SSE1; JEM1 and SSB1; JEM1 and SSB2; JEM1 and ECM10; JEM1 and MDJ1; JEM1 and MDJ2; JEM1 and ERO1; JEM1 and ERV2; JEM1 and EUG1; JEM1 and MPD1; JEM1 and MPD2; JEM1 and EPS1; JEM1 and PDI1; JEM1 and DER1; JEM1 and DER3; JEM1 and HRD3; JEM1 and UBC7; JEM1 and DOA4; JEM1 and HAC1; LHS1 and SCJ1; LHS1 and KAR2; LHS1 and SIL1 LHS1 and FKB2; LHS1 and SSA1; LHS1 and SSA2; LHS1 and SSA3; LHS1 and SSA4; LHS1 and SSE1; LHS1 and SSB1; LHS1 and SSB2; LHS1 and ECM10; LHS1 and MDJ1; LHS1 and MDJ2; LHS1 and ERO1; LHS1 and ERV2; LHS1 and EUG1; LHS1 and MPD1; LHS1 and MPD2; LHS1 and EPS1; LHS1 and PDI1; LHS1 and DER1; LHS1 and DER3; LHS1 and HRD3; LHS1 and UBC7; LHS1 and DOA4; LHS1 and HAC1; SCJ1 and KAR2; SCJ1 and SIL1; SCJ1 and FKB2; SCJ1 and SSA1; SCJ1 and SSA2; SCJ1 and SSA3; SCJ1 and SSA4; SCJ1 and SSE1; SCJ1 and SSB1; SCJ1 and SSB2; SCJ1 and ECM10; SCJ1 and MDJ1; SCJ1 and MDJ2; SCJ1 and ERO1; SCJ1 and ERV2; SCJ1 and EUG1; SCJ1 and MPD1; SCJ1 and MPD2; SCJ1 and EPS1; SCJ1 and PDI1; SCJ1 and DER1; SCJ1 and DER3; SCJ1 and HRD3; SCJ1 and UBC7; SCJ1 and DOA4; SCJ1 and HAC1; KAR2 and SIL1; KAR2 and FKB2; KAR2 and SSA1; KAR2 and SSA2; KAR2 and SSA3; KAR2 and SSA4; KAR2 and SSE1; KAR2 and SSB1; KAR2 and SSB2; KAR2 and ECM10; KAR2 and MDJ1; KAR2 and MDJ2; KAR2 and ERO1; KAR2 and ERV2; KAR2 and EUG1; KAR2 and MPD1; KAR2 and MPD2; KAR2 and EPS1; KAR2 and PDI1; KAR2 and DER1; KAR2 and DER3; KAR2 and HRD3; KAR2 and UBC7; KAR2 and DOA4; KAR2 and HAC1; SIL1 and FKB2; SIL1 and SSA1; SIL1 and SSA2; SIL1 and SSA3; SIL1 and SSA4; SIL1 and SSE1; SIL1 and SSB1; SIL1 and SSB2; SIL1 and ECM10; SIL1 and MDJ1; SIL1 and MDJ2; SIL1 and ERO1; SIL1 and ERV2; SIL1 and EUG1; SIL1 and MPD1; Sail and MPD2; SIL1 and EPS1; SIL1 and PDI1; SIL1 and DER1; SIL1 and DER3; SIL1 and HRD3; SIL1 and UBC7; SIL1 and DOA4; SIL1 and HAC1; FKB2 and SSA1; FKB2 and SSA2; FKB2 and SSA3; FKB2 and SSA4; FKB2 and SSE1; FKB2 and SSB1; FKB2 and SSB2; FKB2 and ECM10; FKB2 and MDJ1; FKB2 and MDJ2; FKB2 and ERO1; FKB2 and ERV2; FKB2 and EUG1; FKB2 and MPD1; FKB2 and MPD2; FKB2 and EPS1; FKB2 and PDI1; FKB2 and DER1; FKB2 and DER3; FKB2 and HRD3; FKB2 and UBC7; FKB2 and DOA4; FKB2 and HAC1; SSA1 and SSA2; SSA1 and SSA3; SSA1 and SSA4; SSA1 and SSE1; SSA1 and SSB1; SSA1 and SSB2; SSA1 and ECM10; SSA1 and MDJ1; SSA1 and MDJ2; SSA1 and ERO1; SSA1 and ERV2; SSA1 and EUG1; SSA1 and MPD1; SSA1 and MPD2; SSA1 and EPS1; SSA1 and PDI1; SSA1 and DER1; SSA1 and DER3; SSA1 and HRD3; SSA1 and UBC7; SSA1 and DOA4; SSA1 and HAC1; SSA2 and SSA3; SSA2 and SSA4; SSA2 and SSE1; SSA2 and SSB1; SSA2 and SSB2; SSA2 and ECM10; SSA2 and MDJ1; SSA2 and MDJ2; SSA2 and ERO1; SSA2 and ERV2; SSA2 and EUG1; SSA2 and MPD1; SSA2 and MPD2; SSA2 and EPS1; SSA2 and PDI1; SSA2 and DER1; SSA2 and DER3; SSA2 and HRD3; SSA2 and UBC7; SSA2 and DOA4; SSA2 and HAC1; SSA3 and SSA4; SSA3 and SSE1; SSA3 and SSB1; SSA3 and SSB2; SSA3 and ECM10; SSA3 and MDJ1; SSA3 and MDJ2; SSA3 and ERO1; SSA3 and ERV2; SSA3 and EUG1; SSA3 and MPD1; SSA3 and MPD2; SSA3 and EPS1; SSA3 and PDI1; SSA3 and DER1; SSA3 and DER3; SSA3 and HRD3; SSA3 and UBC7; SSA3 and DOA4; SSA3 and HAC1; SSA4 and SSE1; SSA4 and SSB1; SSA4 and SSB2; SSA4 and ECM10; SSA4 and MDJ1; SSA4 and MDJ2; SSA4 and ERO1; SSA4 and ERV2; SSA4 and EUG1; SSA4 and MPD1; SSA4 and MPD2; SSA4 and EPS1; SSA4 and PDI1; SSA4 and DER1; SSA4 and DER3; SSA4 and HRD3; SSA4 and UBC7; SSA4 and DOA4; SSA4 and HAC1; SSE1 and SSB1; SSE1 and SSB2; SSE1 and ECM10; SSE1 and MDJ1; SSE1 and MDJ2; SSE1 and ERO1; SSE1 and ERV2; SSE1 and EUG1; SSE1 and MPD1; SSE1 and MPD2; SSE1 and EPS1; SSE1 and PDI1; SSE1 and DER1; SSE1 and DER3; SSE1 and HRD3; SSE1 and UBC7; SSE1 and DOA4; SSE1 and HAC1; SSB1 and SSB2; SSB1 and ECM10; SSB1 and MDJ1; SSB1 and MDJ2; SSB1 and ERO1; SSB1 and ERV2; SSB1 and EUG1; SSB1 and MPD1; SSB1 and MPD2; SSB1 and EPS1; SSB1 and PDI1; SSB1 and DER1; SSB1 and DER3; SSB1 and HRD3; SSB1 and UBC7; SSB1 and DOA4; SSB1 and HAC1; SSB2 and ECM10; SSB2 and MDJ1; SSB2 and MDJ2; SSB2 and ERO1; SSB2 and ERV2; SSB2 and EUG1; SSB2 and MPD1; SSB2 and MPD2; SSB2 and EPS1; SSB2 and PDI1; SSB2 and DER1; SSB2 and DER3; SSB2 and HRD3; SSB2 and UBC7; SSB2 and DOA4; SSB2 and HAC1; ECM10 and MDJ1; ECM10 and MDJ2; ECM10 and ERO1; ECM10 and ERV2; ECM10 and EUG1; ECM10 and MPD1; ECM10 and MPD2; ECM10 and EPS1; ECM10 and PDI1; ECM10 and DER1; ECM10 and DER3; ECM10 and HRD3; ECM10 and UBC7; ECM10 and DOA4; ECM10 and HAC1; MDJ1 and MDJ2; MDJ1 and ERO1; MDJ1 and ERV2; MDJ1 and EUG1; MDJ1 and MPD1; MDJ1 and MPD2; MDJ1 and EPS1; MDJ1 and PDI1; MDJ1 and DER1; MDJ1 and DER3; MDJ1 and HRD3; MDJ1 and UBC7; MDJ1 and DOA4; MDJ1 and HAC1; MDJ2 and ERO1; MDJ2 and ERV2; MDJ2 and EUG1; MDJ2 and MPD1; MDJ2 and MPD2; MDJ2 and EPS1; MDJ2 and PDI1; MDJ2 and DER1; MDJ2 and DER3; MDJ2 and HRD3; MDJ2 and UBC7; MDJ2 and DOA4; MDJ2 and HAC1; ERO1 and ERV2; ERO1 and EUG1; ERO1 and MPD1; ERO1 and MPD2; ERO1 and EPS1; ERO1 and PDI1; ERO1 and DER1; ERO1 and DER3; ERO1 and HRD3; ERO1 and UBC7; ERO1 and DOA4; ERO1 and HAC1; ERV2 and EUG1; ERV2 and MPD1; ERV2 and MPD2; ERV2 and EPS1; ERV2 and PDI1; ERV2 and DER1; ERV2 and DER3; ERV2 and HRD3; ERV2 and UBC7; ERV2 and DOA4; ERV2 and HAC1; EUG1 and MPD1; EUG1 and MPD2; EUG1 and EPS1; EUG1 and PDI1; EUG1 and DER1; EUG1 and DER3; EUG1 and HRD3; EUG1 and UBC7; EUG1 and DOA4; EUG1 and HAC1; MPD1 and MPD2; MPD1 and EPS1; MPD1 and PDI1; MPD1 and DER1; MPD1 and DER3; MPD1 and HRD3; MPD1 and UBC7; MPD1 and DOA4; MPD1 and HAC1; MPD2 and EPS1; MPD2 and PDI1; MPD2 and DER1; MPD2 and DER3; MPD2 and HRD3; MPD2 and UBC7; MPD2 and DOA4; MPD2 and HAC1; EPS1 and PDI1; EPS1 and DER1; EPS1 and DER3; EPS1 and HRD3; EPS1 and UBC7; EPS1 and DOA4; EPS1 and HAC1; PDI1 and DER1; PDI1 and DER3; PDI1 and HRD3; PDI1 and UBC7; PDI1 and DOA4; PDI1 and HAC1; DER1 and DER3; DER1 and HRD3; DER1 and UBC7; DER1 and DOA4; DER1 and HAC1; DER3 and HRD3; DER3 and UBC7; DER3 and DOA4; DER3 and HAC1; HRD3 and UBC7; HRD3 and DOA4; HRD3 and HAC1; UBC7 and DOA4; UBC7 and HAC1; or DOA4 and HAC1.

SSB1 in combination with any one of the following combinations: JEM1 and LHS1; JEM1 and SCJ1; JEM1 and KAR2; JEM1 and SIL1; JEM1 and FKB2; JEM1 and SSA1; JEM1 and SSA2; JEM1 and SSA3; JEM1 and SSA4; JEM1 and SSE1; JEM1 and SSE2; JEM1 and SSB2; JEM1 and ECM10; JEM1 and MDJ1; JEM1 and MDJ2; JEM1 and ERO1; JEM1 and ERV2; JEM1 and EUG1; JEM1 and MPD1; JEM1 and MPD2; JEM1 and EPS1; JEM1 and PDI1; JEM1 and DER1; JEM1 and DER3; JEM1 and HRD3; JEM1 and UBC7; JEM1 and DOA4; JEM1 and HAC1; LHS1 and SCJ1; LHS1 and KAR2; LHS1 and SIL1; LHS1 and FKB2; LHS1 and SSA1; LHS1 and SSA2; LHS1 and SSA3; LHS1 and SSA4; LHS1 and SSE1; LHS1 and SSE2; LHS1 and SSB2; LHS1 and ECM10; LHS1 and MDJ1; LHS1 and MDJ2; LHS1 and ERO1; LHS1 and ERV2; LHS1 and EUG1; LHS1 and MPD1; LHS1 and MPD2; LHS1 and EPS1; LHS1 and PDI1; LHS1 and DER1; LHS1 and DER3; LHS1 and HRD3; LHS1 and UBC7; LHS1 and DOA4; LHS1 and HAC1; SCJ1 and KAR2; SCJ1 and SIL1; SCJ1 and FKB2; SCJ1 and SSA1; SCJ1 and SSA2; SCJ1 and SSA3; SCJ1 and SSA4; SCJ1 and SSE1; SCJ1 and SSE2; SCJ1 and SSB2; SCJ1 and ECM10; SCJ1 and MDJ1; SCJ1 and MDJ2; SCJ1 and ERO1; SCJ1 and ERV2; SCJ1 and EUG1; SCJ1 and MPD1; SCJ1 and MPD2; SCJ1 and EPS1; SCJ1 and PDI1; SCJ1 and DER1; SCJ1 and DER3; SCJ1 and HRD3; SCJ1 and UBC7; SCJ1 and DOA4; SCJ1 and HAC1; KAR2 and SIL1; KAR2 and FKB2; KAR2 and SSA1; KAR2 and SSA2; KAR2 and SSA3; KAR2 and SSA4; KAR2 and SSE1; KAR2 and SSE2; KAR2 and SSB2; KAR2 and ECM10; KAR2 and MDJ1; KAR2 and MDJ2; KAR2 and ERO1; KAR2 and ERV2; KAR2 and EUG1; KAR2 and MPD1; KAR2 and MPD2; KAR2 and EPS1; KAR2 and PDI1; KAR2 and DER1; KAR2 and DER3; KAR2 and HRD3; KAR2 and UBC7; KAR2 and DOA4; KAR2 and HAC1; SIL1 and FKB2; SIL1 and SSA1; SIL1 and SSA2; SIL1 and SSA3; SIL1 and SSA4; SIL1 and SSE1; SIL1 and SSE2; SIL1 and SSB2; SIL1 and ECM10; SIL1 and MDJ1; SIL1 and MDJ2; SIL1 and ERO1; SIL1 and ERV2; SIL1 and EUG1; SIL1 and MPD1; SIL1 and MPD2; SIL1 and EPS1; SIL1 and PDI1; SIL1 and DER1; SIL1 and DER3; SIL1 and HRD3; SIL1 and UBC7; SIL1 and DOA4; SIL1 and HAC1; FKB2 and SSA1; FKB2 and SSA2; FKB2 and SSA3; FKB2 and SSA4; FKB2 and SSE1; FKB2 and SSE2; FKB2 and SSB2; FKB2 and ECM10; FKB2 and MDJ1; FKB2 and MDJ2; FKB2 and ERO1; FKB2 and ERV2; FKB2 and EUG1; FKB2 and MPD1; FKB2 and MPD2; FKB2 and EPS1; FKB2 and PDI1; FKB2 and DER1; FKB2 and DER3; FKB2 and HRD3; FKB2 and UBC7; FKB2 and DOA4; FKB2 and HAC1; SSA1 and SSA2; SSA1 and SSA3; SSA1 and SSA4; SSA1 and SSE1; SSA1 and SSE2; SSA1 and SSB2; SSA1 and ECM10; SSA1 and MDJ1; SSA1 and MDJ2; SSA1 and ERO1; SSA1 and ERV2; SSA1 and EUG1; SSA1 and MPD1; SSA1 and MPD2; SSA1 and EPS1; SSA1 and PDI1; SSA1 and DER1; SSA1 and DER3; SSA1 and HRD3; SSA1 and UBC7; SSA1 and DOA4; SSA1 and HAC1; SSA2 and SSA3; SSA2 and SSA4; SSA2 and SSE1; SSA2 and SSE2; SSA2 and SSB2; SSA2 and ECM10; SSA2 and MDJ1; SSA2 and MDJ2; SSA2 and ERO1; SSA2 and ERV2; SSA2 and EUG1; SSA2 and MPD1; SSA2 and MPD2; SSA2 and EPS1; SSA2 and PDI1; SSA2 and DER1; SSA2 and DER3; SSA2 and HRD3; SSA2 and UBC7; SSA2 and DOA4; SSA2 and HAC1; SSA3 and SSA4; SSA3 and SSE1; SSA3 and SSE2; SSA3 and SSB2; SSA3 and ECM10; SSA3 and MDJ1; SSA3 and MDJ2; SSA3 and ERO1; SSA3 and ERV2; SSA3 and EUG1; SSA3 and MPD1; SSA3 and MPD2; SSA3 and EPS1; SSA3 and PDI1; SSA3 and DER1; SSA3 and DER3; SSA3 and HRD3; SSA3 and UBC7; SSA3 and DOA4; SSA3 and HAC1; SSA4 and SSE1; SSA4 and SSE2; SSA4 and SSB2; SSA4 and ECM10; SSA4 and MDJ1; SSA4 and MDJ2; SSA4 and ERO1; SSA4 and ERV2; SSA4 and EUG1; SSA4 and MPD1; SSA4 and MPD2; SSA4 and EPS1; SSA4 and PDI1; SSA4 and DER1; SSA4 and DER3; SSA4 and HRD3; SSA4 and UBC7; SSA4 and DOA4; SSA4 and HAC1; SSE1 and SSE2; SSE1 and SSB2; SSE1 and ECM10; SSE1 and MDJ1; SSE1 and MDJ2; SSE1 and ERO1; SSE1 and ERV2; SSE1 and EUG1; SSE1 and MPD1; SSE1 and MPD2; SSE1 and EPS1; SSE1 and PDI1; SSE1 and DER1; SSE1 and DER3; SSE1 and HRD3; SSE1 and UBC7; SSE1 and DOA4; SSE1 and HAC1; SSE2 and SSB2; SSE2 and ECM10; SSE2 and MDJ1; SSE2 and MDJ2; SSE2 and ERO1; SSE2 and ERV2; SSE2 and EUG1; SSE2 and MPD1; SSE2 and MPD2; SSE2 and EPS1; SSE2 and PDI1; SSE2 and DER1; SSE2 and DER3; SSE2 and HRD3; SSE2 and UBC7; SSE2 and DOA4; SSE2 and HAC1; SSB2 and ECM10; SSB2 and MDJ1; SSB2 and MDJ2; SSB2 and ERO1; SSB2 and ERV2; SSB2 and EUG1; SSB2 and MPD1; SSB2 and MPD2; SSB2 and EPS1; SSB2 and PDI1; SSB2 and DER1; SSB2 and DER3; SSB2 and HRD3; SSB2 and UBC7; SSB2 and DOA4; SSB2 and HAC1; ECM10 and MDJ1; ECM10 and MDJ2; ECM10 and ERO1; ECM10 and ERV2; ECM10 and EUG1; ECM10 and MPD1; ECM10 and MPD2; ECM10 and EPS1; ECM10 and PDI1; ECM10 and DER1; ECM10 and DER3; ECM10 and HRD3; ECM10 and UBC7; ECM10 and DOA4; ECM10 and HAC1; MDJ1 and MDJ2; MDJ1 and ERO1; MDJ1 and ERV2; MDJ1 and EUG1; MDJ1 and MPD1; MDJ1 and MPD2; MDJ1 and EPS1; MDJ1 and PDI1; MDJ1 and DER1; MDJ1 and DER3; MDJ1 and HRD3; MDJ1 and UBC7; MDJ1 and DOA4; MDJ1 and HAC1; MDJ2 and ERO1; MDJ2 and ERV2; MDJ2 and EUG1; MDJ2 and MPD1; MDJ2 and MPD2; MDJ2 and EPS1; MDJ2 and PDI1; MDJ2 and DER1; MDJ2 and DER3; MDJ2 and HRD3; MDJ2 and UBC7; MDJ2 and DOA4; MDJ2 and HAC1; ERO1 and ERV2; ERO1 and EUG1; ERO1 and MPD1; ERO1 and MPD2; ERO1 and EPS1; ERO1 and PDI1; ERO1 and DER1; ERO1 and DER3; ERO1 and HRD3; ERO1 and UBC7; ERO1 and DOA4; ERO1 and HAC1; ERV2 and EUG1; ERV2 and MPD1; ERV2 and MPD2; ERV2 and EPS1; ERV2 and PDI1; ERV2 and DER1; ERV2 and DER3; ERV2 and HRD3; ERV2 and UBC7; ERV2 and DOA4; ERV2 and HAC1; EUG1 and MPD1; EUG1 and MPD2; EUG1 and EPS1; EUG1 and PDI1; EUG1 and DER1; EUG1 and DER3; EUG1 and HRD3; EUG1 and UBC7; EUG1 and DOA4; EUG1 and HAC1; MPD1 and MPD2; MPD1 and EPS1; MPD1 and PDI1; MPD1 and DER1; MPD1 and DER3; MPD1 and HRD3; MPD1 and UBC7; MPD1 and DOA4; MPD1 and HAC1; MPD2 and EPS1; MPD2 and PDI1; MPD2 and DER1; MPD2 and DER3; MPD2 and HRD3; MPD2 and UBC7; MPD2 and DOA4; MPD2 and HAC1; EPS1 and PDI1; EPS1 and DER1; EPS1 and DER3; EPS1 and HRD3; EPS1 and UBC7; EPS1 and DOA4; EPS1 and HAC1; PDI1 and DER1; PDI1 and DER3; PDI1 and HRD3; PDI1 and UBC7; PDI1 and DOA4; PDI1 and HAC1; DER1 and DER3; DER1 and HRD3; DER1 and UBC7; DER1 and DOA4; DER1 and HAC1; DER3 and HRD3; DER3 and UBC7; DER3 and DOA4; DER3 and HAC1; HRD3 and UBC7; HRD3 and DOA4; HRD3 and HAC1; UBC7 and DOA4; UBC7 and HAC1; or DOA4 and HAC1.

SSB2 in combination with any one of the following combinations: JEM1 and LHS1; JEM1 and SCJ1; JEM1 and KAR2; JEM1 and SIL1; JEM1 and FKB2; JEM1 and SSA1; JEM1 and SSA2; JEM1 and SSA3; JEM1 and SSA4; JEM1 and SSE1; JEM1 and SSE2; JEM1 and SSB1; JEM1 and ECM10; JEM1 and MDJ1; JEM1 and MDJ2; JEM1 and ERO1; JEM1 and ERV2; JEM1 and EUG1; JEM1 and MPD1; JEM1 and MPD2; JEM1 and EPS1; JEM1 and PDI1; JEM1 and DER1; JEM1 and DER3; JEM1 and HRD3; JEM1 and UBC7; JEM1 and DOA4; JEM1 and HAC1; LHS1 and SCJ1; LHS1 and KAR2; LHS1 and SIL1; LHS1 and FKB2; LHS1 and SSA1; LHS1 and SSA2; LHS1 and SSA3; LHS1 and SSA4; LHS1 and SSE1; LHS1 and SSE2; LHS1 and SSB1; LHS1 and ECM10; LHS1 and MDJ1; LHS1 and MDJ2; LHS1 and ERO1; LHS1 and ERV2; LHS1 and EUG1; LHS1 and MPD1; LHS1 and MPD2; LHS1 and EPS1; LHS1 and PDI1; LHS1 and DER1; LHS1 and DER3; LHS1 and HRD3; LHS1 and UBC7; LHS1 and DOA4; LHS1 and HAC1; SCJ1 and KAR2; SCJ1 and SIL1; SCJ1 and FKB2; SCJ1 and SSA1; SCJ1 and SSA2; SCJ1 and SSA3; SCJ1 and SSA4; SCJ1 and SSE1; SCJ1 and SSE2; SCJ1 and SSB1; SCJ1 and ECM10; SCJ1 and MDJ1; SCJ1 and MDJ2; SCJ1 and ERO1; SCJ1 and ERV2; SCJ1 and EUG1; SCJ1 and MPD1; SCJ1 and MPD2; SCJ1 and EPS1; SCJ1 and PDI1; SCJ1 and DER1; SCJ1 and DER3; SCJ1 and HRD3; SCJ1 and UBC7; SCJ1 and DOA4; SCJ1 and HAC1; KAR2 and SIL1; KAR2 and FKB2; KAR2 and SSA1; KAR2 and SSA2; KAR2 and SSA3; KAR2 and SSA4; KAR2 and SSE1; KAR2 and SSE2; KAR2 and SSB1; KAR2 and ECM10; KAR2 and MDJ1; KAR2 and MDJ2; KAR2 and ERO1; KAR2 and ERV2; KAR2 and EUG1; KAR2 and MPD1; KAR2 and MPD2; KAR2 and EPS1; KAR2 and PDI1; KAR2 and DER1; KAR2 and DER3; KAR2 and HRD3; KAR2 and UBC7; KAR2 and DOA4; KAR2 and HAC1; SIL1 and FKB2; SIL1 and SSA1; SIL1 and SSA2; SIL1 and SSA3; SIL1 and SSA4; SIL1 and SSE1; SIL1 and SSE2; SIL1 and SSB1; SIL1 and ECM10; SIL1 and MDJ1; SIL1 and MDJ2; SIL1 and ERO1; SIL1 and ERV2; SIL1 and EUG1; SILT and MPD1; SIL1 and MPD2; SIL1 and EPS1; SIL1 and PDI1; SIL1 and DER1; SIL1 and DER3; SIL1 and HRD3; SIL1 and UBC7; SIL1 and DOA4; SIL1 and HAC1; FKB2 and SSA1; FKB2 and SSA2; FKB2 and SSA3; FKB2 and SSA4; FKB2 and SSE1; FKB2 and SSE2; FKB2 and SSB1; FKB2 and ECM10; FKB2 and MDJ1; FKB2 and MDJ2; FKB2 and ERO1; FKB2 and ERV2; FKB2 and EUG1; FKB2 and MPD1; FKB2 and MPD2; FKB2 and EPS1; FKB2 and PDI1; FKB2 and DER1; FKB2 and DER3; FKB2 and HRD3; FKB2 and UBC7; FKB2 and DOA4; FKB2 and HAC1; SSA1 and SSA2; SSA1 and SSA3; SSA1 and SSA4; SSA1 and SSE1; SSA1 and SSE2; SSA1 and SSB1; SSA1 and ECM10; SSA1 and MDJ1; SSA1 and MDJ2; SSA1 and ERO1; SSA1 and ERV2; SSA1 and EUG1; SSA1 and MPD1; SSA1 and MPD2; SSA1 and EPS1; SSA1 and PDI1; SSA1 and DER1; SSA1 and DER3; SSA1 and HRD3; SSA1 and UBC7; SSA1 and DOA4; SSA1 and HAC1; SSA2 and SSA3; SSA2 and SSA4; SSA2 and SSE1; SSA2 and SSE2; SSA2 and SSB1; SSA2 and ECM10; SSA2 and MDJ1; SSA2 and MDJ2; SSA2 and ERO1; SSA2 and ERV2; SSA2 and EUG1; SSA2 and MPD1; SSA2 and MPD2; SSA2 and EPS1; SSA2 and PDI1; SSA2 and DER1; SSA2 and DER3; SSA2 and HRD3; SSA2 and UBC7; SSA2 and DOA4; SSA2 and HAC1; SSA3 and SSA4; SSA3 and SSE1; SSA3 and SSE2; SSA3 and SSB1; SSA3 and ECM10; SSA3 and MDJ1; SSA3 and MDJ2; SSA3 and ERO1; SSA3 and ERV2; SSA3 and EUG1; SSA3 and MPD1; SSA3 and MPD2; SSA3 and EPS1; SSA3 and PDI1; SSA3 and DER1; SSA3 and DER3; SSA3 and HRD3; SSA3 and UBC7; SSA3 and DOA4; SSA3 and HAC1; SSA4 and SSE1; SSA4 and SSE2; SSA4 and SSB1; SSA4 and ECM10; SSA4 and MDJ1; SSA4 and MDJ2; SSA4 and ERO1; SSA4 and ERV2; SSA4 and EUG1; SSA4 and MPD1; SSA4 and MPD2; SSA4 and EPS1; SSA4 and PDI1; SSA4 and DER1; SSA4 and DER3; SSA4 and HRD3; SSA4 and UBC7; SSA4 and DOA4; SSA4 and HAC1; SSE1 and SSE2; SSE1 and SSB1; SSE1 and ECM10; SSE1 and MDJ1; SSE1 and MDJ2; SSE1 and ERO1; SSE1 and ERV2; SSE1 and EUG1; SSE1 and MPD1; SSE1 and MPD2; SSE1 and EPS1; SSE1 and PDI1; SSE1 and DER1; SSE1 and DER3; SSE1 and HRD3; SSE1 and UBC7; SSE1 and DOA4; SSE1 and HAC1; SSE2 and SSB1; SSE2 and ECM10; SSE2 and MDJ1; SSE2 and MDJ2; SSE2 and ERO1; SSE2 and ERV2; SSE2 and EUG1; SSE2 and MPD1; SSE2 and MPD2; SSE2 and EPS1; SSE2 and PDI1; SSE2 and DER1; SSE2 and DER3; SSE2 and HRD3; SSE2 and UBC7; SSE2 and DOA4; SSE2 and HAC1; SSB1 and ECM10; SSB1 and MDJ1; SSB1 and MDJ2; SSB1 and ERO1; SSB1 and ERV2; SSB1 and EUG1; SSB1 and MPD1; SSB1 and MPD2; SSB1 and EPS1; SSB1 and PDI1; SSB1 and DER1; SSB1 and DER3; SSB1 and HRD3; SSB1 and UBC7; SSB1 and DOA4; SSB1 and HAC1; ECM10 and MDJ1; ECM10 and MDJ2; ECM10 and ERO1; ECM10 and ERV2; ECM10 and EUG1; ECM10 and MPD1; ECM10 and MPD2; ECM10 and EPS1; ECM10 and PDI1; ECM10 and DER1; ECM10 and DER3; ECM10 and HRD3; ECM10 and UBC7; ECM10 and DOA4; ECM10 and HAC1; MDJ1 and MDJ2; MDJ1 and ERO1; MDJ1 and ERV2; MDJ1 and EUG1; MDJ1 and MPD1; MDJ1 and MPD2; MDJ1 and EPS1; MDJ1 and PDI1; MDJ1 and DER1; MDJ1 and DER3; MDJ1 and HRD3; MDJ1 and UBC7; MDJ1 and DOA4; MDJ1 and HAC1; MDJ2 and ERO1; MDJ2 and ERV2; MDJ2 and EUG1; MDJ2 and MPD1; MDJ2 and MPD2; MDJ2 and EPS1; MDJ2 and PDI1; MDJ2 and DER1; MDJ2 and DER3; MDJ2 and HRD3; MDJ2 and UBC7; MDJ2 and DOA4; MDJ2 and HAC1; ERO1 and ERV2; ERO1 and EUG1; ERO1 and MPD1; ERO1 and MPD2; ERO1 and EPS1; ERO1 and PDI1; ERO1 and DER1; ERO1 and DER3; ERO1 and HRD3; ERO1 and UBC7; ERO1 and DOA4; ERO1 and HAC1; ERV2 and EUG1; ERV2 and MPD1; ERV2 and MPD2; ERV2 and EPS1; ERV2 and PDI1; ERV2 and DER1; ERV2 and DER3; ERV2 and HRD3; ERV2 and UBC7; ERV2 and DOA4; ERV2 and HAC1; EUG1 and MPD1; EUG1 and MPD2; EUG1 and EPS1; EUG1 and PDI1; EUG1 and DER1; EUG1 and DER3; EUG1 and HRD3; EUG1 and UBC7; EUG1 and DOA4; EUG1 and HAC1; MPD1 and MPD2; MPD1 and EPS1; MPD1 and PDI1; MPD1 and DER1; MPD1 and DER3; MPD1 and HRD3; MPD1 and UBC7; MPD1 and DOA4; MPD1 and HAC1; MPD2 and EPS1; MPD2 and PDI1; MPD2 and DER1; MPD2 and DER3; MPD2 and HRD3; MPD2 and UBC7; MPD2 and DOA4; MPD2 and HAC1; EPS1 and PDI1; EPS1 and DER1; EPS1 and DER3; EPS1 and HRD3; EPS1 and UBC7; EPS1 and DOA4; EPS1 and HAC1; PDI1 and DER1; PDI1 and DER3; PDI1 and HRD3; PDI1 and UBC7; PDI1 and DOA4; PDI1 and HAC1; DER1 and DER3; DER1 and HRD3; DER1 and UBC7; DER1 and DOA4; DER1 and HAC1; DER3 and HRD3; DER3 and UBC7; DER3 and DOA4; DER3 and HAC1; HRD3 and UBC7; HRD3 and DOA4; HRD3 and HAC1; UBC7 and DOA4; UBC7 and HAC1; or DOA4 and HAC1.

ECM10 in combination with any one of the following combinations: JEM1 and LHS1; JEM1 and SCJ1; JEM1 and KAR2; JEM1 and SIL1; JEM1 and FKB2; JEM1 and SSA1; EMI and SSA2; JEM1 and SSA3; JEM1 and SSA4; JEM1 and SSE1; JEM1 and SSE2; JEM1 and SSB1; JEM1 and SSB2; JEM1 and MDJ1; JEM1 and MDJ2; JEM1 and ERO1; JEM1 and ERV2; JEM1 and EUG1; JEM1 and MPD1; JEM1 and MPD2; JEM1 and EPS1; JEM1 and PDI1; JEM1 and DER1; JEM1 and DER3; JEM1 and HRD3; JEM1 and UBC7; JEW and DOA4; JEM1 and HAC1; LHS1 and SCJ1; LHS1 and KAR2; LHS1 and SIL1 LHS1 and FKB2; LHS1 and SSA1; LHS1 and SSA2; LHS1 and SSA3; LHS1 and SSA4; LHS1 and SSE1; LHS1 and SSE2; LHS1 and SSB1; LHS1 and SSB2; LHS1 and MDJ1; LHS1 and MDJ2; LHS1 and ERO1; LHS1 and ERV2; LHS1 and EUG1; LHS1 and MPD1; LHS1 and MPD2; LHS1 and EPS1; LHS1 and PDI1; LHS1 and DER1; LHS1 and DER3; LHS1 and HRD3; LHS1 and UBC7; LHS1 and DOA4; LHS1 and HAC1; SCJ1 and KAR2; SCJ1 and SID; SCJ1 and FKB2; SCJ1 and SSA1; SCJ1 and SSA2; SCJ1 and SSA3; SCJ1 and SSA4; SCJ1 and SSE1; SCJ1 and SSE2; SCJ1 and SSB1; SCJ1 and SSB2; SCJ1 and MDJ1; SCJ1 and MDJ2; SCJ1 and ERO1; SCJ1 and ERV2; SCJ1 and EUG1; SCJ1 and MPD1; SCJ1 and MPD2; SCJ1 and EPS1; SCJ1 and PDI1; SCJ1 and DER1; SCJ1 and DER3; SCJ1 and HRD3; SCJ1 and UBC7; SCJ1 and DOA4; SCJ1 and HAC1; KAR2 and SIL1; KAR2 and FKB2; KAR2 and SSA1; KAR2 and SSA2; KAR2 and SSA3; KAR2 and SSA4; KAR2 and SSE1; KAR2 and SSE2; KAR2 and SSB1; KAR2 and SSB2; KAR2 and MDJ1; KAR2 and MDJ2; KAR2 and ERO1; KAR2 and ERV2; KAR2 and EUG1; KAR2 and MPD1; KAR2 and MPD2; KAR2 and EPS1; KAR2 and PDI1; KAR2 and DER1; KAR2 and DER3; KAR2 and HRD3; KAR2 and UBC7; KAR2 and DOA4; KAR2 and HAC1; SIL1 and FKB2; SIL1 and SSA1; SIL1 and SSA2; SIL1 and SSA3; SIL1 and SSA4; SIL1 and SSE1; SIL1 and SSE2; SILT and SSB1; SIL1 and SSB2; SIL1 and MDJ1; SIL1 and MDJ2; SIL1 and ERO1; SIL1 and ERV2; SIL1 and EUG1; SIL1 and MPD1; SIL1 and MPD2; SIL1 and EPS1; SIL1 and PDI1; SIL1 and DER1; SIL1 and DER3; SIL1 and HRD3; SIL1 and UBC7; SIL1 and DOA4; SIL1 and HAC1; FKB2 and SSA1; FKB2 and SSA2; FKB2 and SSA3; FKB2 and SSA4; FKB2 and SSE1; FKB2 and SSE2; FKB2 and SSB1; FKB2 and SSB2; FKB2 and MDJ1; FKB2 and MDJ2; FKB2 and ERO1; FKB2 and ERV2; FKB2 and EUG1; FKB2 and MPD1; FKB2 and MPD2; FKB2 and EPS1; FKB2 and PDI1; FKB2 and DER1; FKB2 and DER3; FKB2 and HRD3; FKB2 and UBC7; FKB2 and DOA4; FKB2 and HAC1; SSA1 and SSA2; SSA1 and SSA3; SSA1 and SSA4; SSA1 and SSE1; SSA1 and SSE2; SSA1 and SSB1; SSA1 and SSB2; SSA1 and MDJ1; SSA1 and MDJ2; SSA1 and ERO1; SSA1 and ERV2; SSA1 and EUG1; SSA1 and MPD1; SSA1 and MPD2; SSA1 and EPS1; SSA1 and PDI1; SSA1 and DER1; SSA1 and DER3; SSA1 and HRD3; SSA1 and UBC7; SSA1 and DOA4; SSA1 and HAC1; SSA2 and SSA3; SSA2 and SSA4; SSA2 and SSE1; SSA2 and SSE2; SSA2 and SSB1; SSA2 and SSB2; SSA2 and MDJ1; SSA2 and MDJ2; SSA2 and ERO1; SSA2 and ERV2; SSA2 and EUG1; SSA2 and MPD1; SSA2 and MPD2; SSA2 and EPS1; SSA2 and PDI1; SSA2 and DER1; SSA2 and DER3; SSA2 and HRD3; SSA2 and UBC7; SSA2 and DOA4; SSA2 and HAC1; SSA3 and SSA4; SSA3 and SSE1; SSA3 and SSE2; SSA3 and SSB1; SSA3 and SSB2; SSA3 and MDJ1; SSA3 and MDJ2; SSA3 and ERO1; SSA3 and ERV2; SSA3 and EUG1; SSA3 and MPD1; SSA3 and MPD2; SSA3 and EPS1; SSA3 and PDI1; SSA3 and DER1; SSA3 and DER3; SSA3 and HRD3; SSA3 and UBC7; SSA3 and DOA4; SSA3 and HAC1; SSA4 and SSE1; SSA4 and SSE2; SSA4 and SSB1; SSA4 and SSB2; SSA4 and MDJ1; SSA4 and MDJ2; SSA4 and ERO1; SSA4 and ERV2; SSA4 and EUG1; SSA4 and MPD1; SSA4 and MPD2; SSA4 and EPS1; SSA4 and PDI1; SSA4 and DER1; SSA4 and DER3; SSA4 and HRD3; SSA4 and UBC7; SSA4 and DOA4; SSA4 and HAC1; SSE1 and SSE2; SSE1 and SSB1; SSE1 and SSB2; SSE1 and MDJ1; SSE1 and MDJ2; SSE1 and ERO1; SSE1 and ERV2; SSE1 and EUG1; SSE1 and MPD1; SSE1 and MPD2; SSE1 and EPS1; SSE1 and PDI1; SSE1 and DER1; SSE1 and DER3; SSE1 and HRD3; SSE1 and UBC7; SSE1 and DOA4; SSE1 and HAC1; SSE2 and SSB1; SSE2 and SSB2; SSE2 and MDJ1; SSE2 and MDJ2; SSE2 and ERO1; SSE2 and ERV2; SSE2 and EUG1; SSE2 and MPD1; SSE2 and MPD2; SSE2 and EPS1; SSE2 and PDI1; SSE2 and DER1; SSE2 and DER3; SSE2 and HRD3; SSE2 and UBC7; SSE2 and DOA4; SSE2 and HAC1; SSB1 and SSB2; SSB1 and MDJ1; SSB1 and MDJ2; SSB1 and ERO1; SSB1 and ERV2; SSB1 and EUG1; SSB1 and MPD1; SSB1 and MPD2; SSB1 and EPS1; SSB1 and PDI1; SSB1 and DER1; SSB1 and DER3; SSB1 and HRD3; SSB1 and UBC7; SSB1 and DOA4; SSB1 and HAC1; SSB2 and MDJ1; SSB2 and MDJ2; SSB2 and ERO1; SSB2 and ERV2; SSB2 and EUG1; SSB2 and MPD1; SSB2 and MPD2; SSB2 and EPS1; SSB2 and PDI1; SSB2 and DER1; SSB2 and DER3; SSB2 and HRD3; SSB2 and UBC7; SSB2 and DOA4; SSB2 and HAC1; MDJ1 and MDJ2; MDJ1 and ERO1; MDJ1 and ERV2; MDJ1 and EUG1; MDJ1 and MPD1; MDJ1 and MPD2; MDJ1 and EPS1; MDJ1 and PDI1; MDJ1 and DER1; MDJ1 and DER3; MDJ1 and HRD3; MDJ1 and UBC7; MDJ1 and DOA4; MDJ1 and HAC1; MDJ2 and ERO1; MDJ2 and ERV2; MDJ2 and EUG1; MDJ2 and MPD1; MDJ2 and MPD2; MDJ2 and EPS1; MDJ2 and PDI1; MDJ2 and DER1; MDJ2 and DER3; MDJ2 and HRD3; MDJ2 and UBC7; MDJ2 and DOA4; MDJ2 and HAC1; ERO1 and ERV2; ERO1 and EUG1; ERO1 and MPD1; ERO1 and MPD2; ERO1 and EPS1; ERO1 and PDI1; ERO1 and DER1; ERO1 and DER3; ERO1 and HRD3; ERO1 and UBC7; ERO1 and DOA4; ERO1 and HAC1; ERV2 and EUG1; ERV2 and MPD1; ERV2 and MPD2; ERV2 and EPS1; ERV2 and PDI1; ERV2 and DER1; ERV2 and DER3; ERV2 and HRD3; ERV2 and UBC7; ERV2 and DOA4; ERV2 and HAC1; EUG1 and MPD1; EUG1 and MPD2; EUG1 and EPS1; EUG1 and PDI1; EUG1 and DER1; EUG1 and DER3; EUG1 and HRD3; EUG1 and UBC7; EUG1 and DOA4; EUG1 and HAC1; MPD1 and MPD2; MPD1 and EPS1; MPD1 and PDI1; MPD1 and DER1; MPD1 and DER3; MPD1 and HRD3; MPD1 and UBC7; MPD1 and DOA4; MPD1 and HAC1; MPD2 and EPS1; MPD2 and PDI1; MPD2 and DER1; MPD2 and DER3; MPD2 and HRD3; MPD2 and UBC7; MPD2 and DOA4; MPD2 and HAC1; EPS1 and PDI1; EPS1 and DER1; EPS1 and DER3; EPS1 and HRD3; EPS1 and UBC7; EPS1 and DOA4; EPS1 and HAC1; PDI1 and DER1; PDI1 and DER3; PDI1 and HRD3; PDI1 and UBC7; PDI1 and DOA4; PDI1 and HAC1; DER1 and DER3; DER1 and HRD3; DER1 and UBC7; DER1 and DOA4; DER1 and HAC1; DER3 and HRD3; DER3 and UBC7; DER3 and DOA4; DER3 and HAC1; HRD3 and UBC7; HRD3 and DOA4; HRD3 and HAC1; UBC7 and DOA4; UBC7 and HAC1; or DOA4 and HAC1.

MDJ1 in combination with any one of the following combinations: JEM1 and LHS1; JEM1 and SCJ1; JEM1 and KAR2; JEM1 and SIL1; JEM1 and FKB2; JEM1 and SSA1; JEM1 and SSA2; JEM1 and SSA3; JEM1 and SSA4; JEM1 and SSE1; JEM1 and SSE2; JEM1 and SSB1; JEM1 and SSB2; JEM1 and ECM10; JEM1 and MDJ2; JEM1 and ERO1; JEM1 and ERV2; JEM1 and EUG1; JEM1 and MPD1; JEM1 and MPD2; JEM1 and EPS1; JEM1 and PDI1; JEM1 and DER1; JEM1 and DER3; JEM1 and HRD3; JEM1 and UBC7; JEM1 and DOA4; JEM1 and HAC1; LHS1 and SCJ1; LHS1 and KAR2; LHS1 and SIL1; LHS1 and FKB2; LHS1 and SSA1; LHS1 and SSA2; LHS1 and SSA3; LHS1 and SSA4; LHS1 and SSE1; LHS1 and SSE2; LHS1 and SSB1; LHS1 and SSB2; LHS1 and ECM10; LHS1 and MDJ2; LHS1 and ERO1; LHS1 and ERV2; LHS1 and EUG1; LHS1 and MPD1; LHS1 and MPD2; LHS1 and EPS1; LHS1 and PDI1; LHS1 and DER1; LHS1 and DER3; LHS1 and HRD3; LHS1 and UBC7; LHS1 and DOA4; LHS1 and HAC1; SCJ1 and KAR2; SCJ1 and SIL1; SCJ1 and FKB2; SCJ1 and SSA1; SCJ1 and SSA2; SCJ1 and SSA3; SCJ1 and SSA4; SCJ1 and SSE1; SCJ1 and SSE2; SCJ1 and SSB1; SCJ1 and SSB2; SCJ1 and ECM10; SCJ1 and MDJ2; SCJ1 and ERO1; SCJ1 and ERV2; SCJ1 and EUG1; SCJ1 and MPD1; SCJ1 and MPD2; SCJ1 and EPS1; SCJ1 and PDI1; SCJ1 and DER1; SCJ1 and DER3; SCJ1 and HRD3; SCJ1 and UBC7; SCJ1 and DOA4; SCJ1 and HAC1; KAR2 and SIL1; KAR2 and FKB2; KAR2 and SSA1; KAR2 and SSA2; KAR2 and SSA3; KAR2 and SSA4; KAR2 and SSE1; KAR2 and SSE2; KAR2 and SSB1; KAR2 and SSE2; KAR2 and ECM10; KAR2 and MDJ2; KAR2 and ERO1; KAR2 and ERV2; KAR2 and EUG1; KAR2 and MPD1; KAR2 and MPD2; KAR2 and EPS1; KAR2 and PDI1; KAR2 and DER1; KAR2 and DER3; KAR2 and HRD3; KAR2 and UBC7; KAR2 and DOA4; KAR2 and HAC1; SIL1 and FKB2; SIL1 and SSA1; SIL1 and SSA2; SIL1 and SSA3; SIL1 and SSA4; SIL1 and SSE1; SIL1 and SSE2; SIL1 and SSB1; SIL1 and SSB2; SIL1 and ECM10; SIL1 and MDJ2; SIL1 and ERO1; SIL1 and ERV2; SIL1 and EUG1; SIL1 and MPD1; SIL1 and MPD2; SIL1 and EPS1; SIL1 and PDI1; SIL1 and DER1; SIL1 and DER3; SIL1 and HRD3; SIL1 and UBC7; SIL1 and DOA4; SIL1 and HAC1; FKB2 and SSA1; FKB2 and SSA2; FKB2 and SSA3; FKB2 and SSA4; FKB2 and SSE1; FKB2 and SSE2; FKB2 and SSB1; FKB2 and SSB2; FKB2 and ECM10; FKB2 and MDJ2; FKB2 and ERO1; FKB2 and ERV2; FKB2 and EUG1; FKB2 and MPD1; FKB2 and MPD2; FKB2 and EPS1; FKB2 and PDI1; FKB2 and DER1; FKB2 and DER3; FKB2 and HRD3; FKB2 and UBC7; FKB2 and DOA4; FKB2 and HAC1; SSA1 and SSA2; SSA1 and SSA3; SSA1 and SSA4; SSA1 and SSE1; SSA1 and SSE2; SSA1 and SSB1; SSA1 and SSB2; SSA1 and ECM10; SSA1 and MDJ2; SSA1 and ERO1; SSA1 and ERV2; SSA1 and EUG1; SSA1 and MPD1; SSA1 and MPD2; SSA1 and EPS1; SSA1 and PDI1; SSA1 and DER1; SSA1 and DER3; SSA1 and HRD3; SSA1 and UBC7; SSA1 and DOA4; SSA1 and HAC1; SSA2 and SSA3; SSA2 and SSA4; SSA2 and SSE1; SSA2 and SSE2; SSA2 and SSB1; SSA2 and SSB2; SSA2 and ECM10; SSA2 and MDJ2; SSA2 and ERO1; SSA2 and ERV2; SSA2 and EUG1; SSA2 and MPD1; SSA2 and MPD2; SSA2 and EPS1; SSA2 and PDI1; SSA2 and DER1; SSA2 and DER3; SSA2 and HRD3; SSA2 and UBC7; SSA2 and DOA4; SSA2 and HAC1; SSA3 and SSA4; SSA3 and SSE1; SSA3 and SSE2; SSA3 and SSB1; SSA3 and SSB2; SSA3 and ECM10; SSA3 and MDJ2; SSA3 and ERO1; SSA3 and ERV2; SSA3 and EUG1; SSA3 and MPD1; SSA3 and MPD2; SSA3 and EPS1; SSA3 and PDI1; SSA3 and DER1; SSA3 and DER3; SSA3 and HRD3; SSA3 and UBC7; SSA3 and DOA4; SSA3 and HAC1; SSA4 and SSE1; SSA4 and SSE2; SSA4 and SSB1; SSA4 and SSB2; SSA4 and ECM10; SSA4 and MDJ2; SSA4 and ERO1; SSA4 and ERV2; SSA4 and EUG1; SSA4 and MPD1; SSA4 and MPD2; SSA4 and EPS1; SSA4 and PDI1; SSA4 and DER1; SSA4 and DER3; SSA4 and HRD3; SSA4 and UBC7; SSA4 and DOA4; SSA4 and HAC1; SSE1 and SSE2; SSE1 and SSB1; SSE1 and SSB2; SSE1 and ECM10; SSE1 and MDJ2; SSE1 and ERO1; SSE1 and ERV2; SSE1 and EUG1; SSE1 and MPD1; SSE1 and MPD2; SSE1 and EPS1; SSE1 and PDI1; SSE1 and DER1; SSE1 and DER3; SSE1 and HRD3; SSE1 and UBC7; SSE1 and DOA4; SSE1 and HAC1; SSE2 and SSB1; SSE2 and SSB2; SSE2 and ECM10; SSE2 and MDJ2; SSE2 and ERO1; SSE2 and ERV2; SSE2 and EUG1; SSE2 and MPD1; SSE2 and MPD2; SSE2 and EPS1; SSE2 and PDI1; SSE2 and DER1; SSE2 and DER3; SSE2 and HRD3; SSE2 and UBC7; SSE2 and DOA4; SSE2 and HAC1; SSB1 and SSB2; SSB1 and ECM10; SSB1 and MDJ2; SSB1 and ERO1; SSB1 and ERV2; SSB1 and EUG1; SSB1 and MPD1; SSB1 and MPD2; SSB1 and EPS1; SSB1 and PDI1; SSB1 and DER1; SSB1 and DER3; SSB1 and HRD3; SSB1 and UBC7; SSB1 and DOA4; SSB1 and HAC1; SSB2 and ECM10; SSB2 and MDJ2; SSB2 and ERO1; SSB2 and ERV2; SSB2 and EUG1; SSB2 and MPD1; SSB2 and MPD2; SSB2 and EPS1; SSB2 and PDI1; SSB2 and DER1; SSB2 and DER3; SSB2 and HRD3; SSB2 and UBC7; SSB2 and DOA4; SSB2 and HAC1; ECM10 and MDJ2; ECM10 and ERO1; ECM10 and ERV2; ECM10 and EUG1; ECM10 and MPD1; ECM10 and MPD2; ECM10 and EPS1; ECM10 and PDI1; ECM10 and DER1; ECM10 and DER3; ECM10 and HRD3; ECM10 and UBC7; ECM10 and DOA4; ECM10 and HAC1; MDJ2 and ERO1; MDJ2 and ERV2; MDJ2 and EUG1; MDJ2 and MPD1; MDJ2 and MPD2; MDJ2 and EPS1; MDJ2 and PDI1; MDJ2 and DER1; MDJ2 and DER3; MDJ2 and HRD3; MDJ2 and UBC7; MDJ2 and DOA4; MDJ2 and HAC1; ERO1 and ERV2; ERO1 and EUG1; ERO1 and MPD1; ERO1 and MPD2; ERO1 and EPS1; ERO1 and PDI1; ERO1 and DER1; ERO1 and DER3; ERO1 and HRD3; ERO1 and UBC7; ERO1 and DOA4; ERO1 and HAC1; ERV2 and EUG1; ERV2 and MPD1; ERV2 and MPD2; ERV2 and EPS1; ERV2 and PDI1; ERV2 and DER1; ERV2 and DER3; ERV2 and HRD3; ERV2 and UBC7; ERV2 and DOA4; ERV2 and HAC1; EUG1 and MPD1; EUG1 and MPD2; EUG1 and EPS1; EUG1 and PDI1; EUG1 and DER1; EUG1 and DER3; EUG1 and HRD3; EUG1 and UBC7; EUG1 and DOA4; EUG1 and HAC1; MPD1 and MPD2; MPD1 and EPS1; MPD1 and PDI1; MPD1 and DER1; MPD1 and DER3; MPD1 and HRD3; MPD1 and UBC7; MPD1 and DOA4; MPD1 and HAC1; MPD2 and EPS1; MPD2 and PDI1; MPD2 and DER1; MPD2 and DER3; MPD2 and HRD3; MPD2 and UBC7; MPD2 and DOA4; MPD2 and HAC1; EPS1 and PDI1; EPS1 and DER1; EPS1 and DER3; EPS1 and HRD3; EPS1 and UBC7; EPS1 and DOA4; EPS1 and HAC1; PDI1 and DER1; PDI1 and DER3; PDI1 and HRD3; PDI1 and UBC7; PDI1 and DOA4; PDI1 and HAC1; DER1 and DER3; DER1 and HRD3; DER1 and UBC7; DER1 and DOA4; DER1 and HAC1; DER3 and HRD3; DER3 and UBC7; DER3 and DOA4; DER3 and HAC1; HRD3 and UBC7; HRD3 and DOA4; HRD3 and HAC1; UBC7 and DOA4; UBC7 and HAC1; or DOA4 and HAC1.

MDJ2 in combination with any one of the following combinations: JEM1 and LHS1; JEM1 and SCJ1; JEM1 and KAR2; JEM1 and SIL1; JEM1 and FKB2; JEM1 and SSA1; JEM1 and SSA2; JEM1 and SSA3; JEM1 and SSA4; JEM1 and SSE1; JEM1 and SSE2; JEM1 and SSB1; JEM1 and SSB2; JEM1 and ECM10; JEM1 and MDJ1; JEM1 and ERO1; JEM1 and ERV2; JEM1 and EUG1; JEM1 and MPD1; JEM1 and MPD2; JEM1 and EPS1; JEM1 and PDI1; JEM1 and DER1; JEM1 and DER3; JEM1 and HRD3; JEM1 and UBC7; JEM1 and DOA4; JEM1 and HAC1; LHS1 and SCJ1; LHS1 and KAR2; LHS1 and SIL1; LHS1 and FKB2; LHS1 and SSA1; LHS1 and SSA2; LHS1 and SSA3; LHS1 and SSA4; LHS1 and SSE1; LHS1 and SSE2; LHS1 and SSB1; LHS1 and SSB2; LHS1 and ECM10; LHS1 and MDJ1; LHS1 and ERO1; LHS1 and ERV2; LHS1 and EUG1; LHS1 and MPD1; LHS1 and MPD2; LHS1 and EPS1; LHS1 and PDI1; LHS1 and DER1; LHS1 and DER3; LHS1 and HRD3; LHS1 and UBC7; LHS1 and DOA4; LHS1 and HAC1; SCJ1 and KAR2; SCJ1 and SIL1; SCJ1 and FKB2; SCJ1 and SSA1; SCJ1 and SSA2; SCJ1 and SSA3; SCJ1 and SSA4; SCJ1 and SSE1; SCJ1 and SSE2; SCJ1 and SSB1; SCJ1 and SSB2; SCJ1 and ECM10; SCJ1 and MDJ1; SCJ1 and ERO1; SCJ1 and ERV2; SCJ1 and EUG1; SCJ1 and MPD1; SCJ1 and MPD2; SCJ1 and EPS1; SCJ1 and PDI1; SCJ1 and DER1; SCJ1 and DER3; SCJ1 and HRD3; SCJ1 and UBC7; SCJ1 and DOA4; SCJ1 and HAC1; KAR2 and SIL1; KAR2 and FKB2; KAR2 and SSA1; KAR2 and SSA2; KAR2 and SSA3; KAR2 and SSA4; KAR2 and SSE1; KAR2 and SSE2; KAR2 and SSB1; KAR2 and SSB2; KAR2 and ECM10; KAR2 and MDJ1; KAR2 and ERO1; KAR2 and ERV2; KAR2 and EUG1; KAR2 and MPD1; KAR2 and MPD2; KAR2 and EPS1; KAR2 and PDI1; KAR2 and DER1; KAR2 and DER3; KAR2 and HRD3; KAR2 and UBC7; KAR2 and DOA4; KAR2 and HAC1; SIL1 and FKB2; SIL1 and SSA1; SIL1 and SSA2; SIL1 and SSA3; SIL1 and SSA4; SIL1 and SSE1; SIL1 and SSE2; SIL1 and SSB1; SIL1 and SSB2; SIL1 and ECM10; SIL1 and MDJ1; SIL1 and ERO1; SIL1 and ERV2; SIL1 and EUG1; SIL1 and MPD1; SIL1 and MPD2; SIL1 and EPS1; SIL1 and PDI1; SIL1 and DER1; SIL1 and DER3; SIL1 and HRD3; SIL1 and UBC7; SIL1 and DOA4; SIL1 and HAC1; FKB2 and SSA1; FKB2 and SSA2; FKB2 and SSA3; FKB2 and SSA4; FKB2 and SSE1; FKB2 and SSE2; FKB2 and SSB1; FKB2 and SSB2; FKB2 and ECM10; FKB2 and MDJ1; FKB2 and ERO1; FKB2 and ERV2; FKB2 and EUG1; FKB2 and MPD1; FKB2 and MPD2; FKB2 and EPS1; FKB2 and PDI1; FKB2 and DER1; FKB2 and DER3; FKB2 and HRD3; FKB2 and UBC7; FKB2 and DOA4; FKB2 and HAC1; SSA1 and SSA2; SSA1 and SSA3; SSA1 and SSA4; SSA1 and SSE1; SSA1 and SSE2; SSA1 and SSB1; SSA1 and SSB2; SSA1 and ECM10; SSA1 and MDJ1; SSA1 and ERO1; SSA1 and ERV2; SSA1 and EUG1; SSA1 and MPD1; SSA1 and MPD2; SSA1 and EPS1; SSA1 and PDI1; SSA1 and DER1; SSA1 and DER3; SSA1 and HRD3; SSA1 and UBC7; SSA1 and DOA4; SSA1 and HAC1; SSA2 and SSA3; SSA2 and SSA4; SSA2 and SSE1; SSA2 and SSE2; SSA2 and SSB1; SSA2 and SSB2; SSA2 and ECM10; SSA2 and MDJ1; SSA2 and ERO1; SSA2 and ERV2; SSA2 and EUG1; SSA2 and MPD1; SSA2 and MPD2; SSA2 and EPS1; SSA2 and PDI1; SSA2 and DER1; SSA2 and DER3; SSA2 and HRD3; SSA2 and UBC7; SSA2 and DOA4; SSA2 and HAC1; SSA3 and SSA4; SSA3 and SSE1; SSA3 and SSE2; SSA3 and SSB1; SSA3 and SSB2; SSA3 and ECM10; SSA3 and MDJ1; SSA3 and ERO1; SSA3 and ERV2; SSA3 and EUG1; SSA3 and MPD1; SSA3 and MPD2; SSA3 and EPS1; SSA3 and PDI1; SSA3 and DER1; SSA3 and DER3; SSA3 and HRD3; SSA3 and UBC7; SSA3 and DOA4; SSA3 and HAC1; SSA4 and SSE1; SSA4 and SSE2; SSA4 and SSB1; SSA4 and SSB2; SSA4 and ECM10; SSA4 and MDJ1; SSA4 and ERO1; SSA4 and ERV2; SSA4 and EUG1; SSA4 and MPD1; SSA4 and MPD2; SSA4 and EPS1; SSA4 and PDI1; SSA4 and DER1; SSA4 and DER3; SSA4 and HRD3; SSA4 and UBC7; SSA4 and DOA4; SSA4 and HAC1; SSE1 and SSE2; SSE1 and SSB1; SSE1 and SSB2; SSE1 and ECM10; SSE1 and MDJ1; SSE1 and ERO1; SSE1 and ERV2; SSE1 and EUG1; SSE1 and MPD1; SSE1 and MPD2; SSE1 and EPS1; SSE1 and PDI1; SSE1 and DER1; SSE1 and DER3; SSE1 and HRD3; SSE1 and UBC7; SSE1 and DOA4; SSE1 and HAC1; SSE2 and SSB1; SSE2 and SSB2; SSE2 and ECM10; SSE2 and MDJ1; SSE2 and ERO1; SSE2 and ERV2; SSE2 and EUG1; SSE2 and MPD1; SSE2 and MPD2; SSE2 and EPS1; SSE2 and PDI1; SSE2 and DER1; SSE2 and DER3; SSE2 and HRD3; SSE2 and UBC7; SSE2 and DOA4; SSE2 and HAC1; SSB1 and SSB2; SSB1 and ECM10; SSB1 and MDJ1; SSB1 and ERO1; SSB1 and ERV2; SSB1 and EUG1; SSB1 and MPD1; SSB1 and MPD2; SSB1 and EPS1; SSB1 and PDI1; SSB1 and DER1; SSB1 and DER3; SSB1 and HRD3; SSB1 and UBC7; SSB1 and DOA4; SSB1 and HAC1; SSB2 and ECM10; SSB2 and MDJ1; SSB2 and ERO1; SSB2 and ERV2; SSB2 and EUG1; SSB2 and MPD1; SSB2 and MPD2; SSB2 and EPS1; SSB2 and PDI1; SSB2 and DER1; SSB2 and DER3; SSB2 and HRD3; SSB2 and UBC7; SSB2 and DOA4; SSB2 and HAC1; ECM10 and MDJ1; ECM10 and ERO1; ECM10 and ERV2; ECM10 and EUG1; ECM10 and MPD1; ECM10 and MPD2; ECM10 and EPS1; ECM10 and PDI1; ECM10 and DER1; ECM10 and DER3; ECM10 and HRD3; ECM10 and UBC7; ECM10 and DOA4; ECM10 and HAC1; MDJ1 and ERO1; MDJ1 and ERV2; MDJ1 and EUG1; MDJ1 and MPD1; MDJ1 and MPD2; MDJ1 and EPS1; MDJ1 and PDI1; MDJ1 and DER1; MDJ1 and DER3; MDJ1 and HRD3; MDJ1 and UBC7; MDJ1 and DOA4; MDJ1 and HAC1; ERO1 and ERV2; ERO1 and EUG1; ERO1 and MPD1; ERO1 and MPD2; ERO1 and EPS1; ERO1 and PDI1; ERO1 and DER1; ERO1 and DER3; ERO1 and HRD3; ERO1 and UBC7; ERO1 and DOA4; ERO1 and HAC1; ERV2 and EUG1; ERV2 and MPD1; ERV2 and MPD2; ERV2 and EPS1; ERV2 and PDI1; ERV2 and DER1; ERV2 and DER3; ERV2 and HRD3; ERV2 and UBC7; ERV2 and DOA4; ERV2 and HAC1; EUG1 and MPD1; EUG1 and MPD2; EUG1 and EPS1; EUG1 and PDI1; EUG1 and DER1; EUG1 and DER3; EUG1 and HRD3; EUG1 and UBC7; EUG1 and DOA4; EUG1 and HAC1; MPD1 and MPD2; MPD1 and EPS1; MPD1 and PDI1; MPD1 and DER1; MPD1 and DER3; MPD1 and HRD3′; MPD1 and UBC7; MPD1 and DOA4; MPD1 and HAC1; MPD2 and EPS1; MPD2 and PDI1; MPD2 and DER1; MPD2 and DER3; MPD2 and HRD3; MPD2 and UBC7; MPD2 and DOA4; MPD2 and HAC1; EPS1 and PDI1; EPS1 and DER1; EPS1 and DER3; EPS1 and HRD3; EPS1 and UBC7; EPS1 and DOA4; EPS1 and HAC1; PDI1 and DER1; PDI1 and DER3; PDI1 and HRD3; PDI1 and UBC7; PDI1 and DOA4; PDI1 and HAC1; DER1 and DER3; DER1 and HRD3; DER1 and UBC7; DER1 and DOA4; DER1 and HAC1; DER3 and HRD3; DER3 and UBC7; DER3 and DOA4; DER3 and HAC1; HRD3 and UBC7; HRD3 and DOA4; HRD3 and HAC1; UBC7 and DOA4; UBC7 and HAC1; or DOA4 and HAC1.

ERO1 in combination with any one of the following combinations: JEM1 and LHS1; JEM1 and SCJ1; JEM1 and KAR2; JEM1 and SIL1; JEM1 and FKB2; JEM1 and SSA1; JEM1 and SSA2; JEM1 and SSA3; JEM1 and SSA4; JEM1 and SSE1; JEM1 and SSE2; JEM1 and SSB1; JEM1 and SSB2; JEM1 and ECM10; JEM1 and MDJ1; JEM1 and MDJ2; JEM1 and ERV2; JEM1 and EUG1; JEM1 and MPD1; JEM1 and MPD2; JEM1 and EPS1; JEM1 and PDI1; JEM1 and DER1; JEM1 and DER3; JEM1 and HRD3; JEM1 and UBC7; JEM1 and DOA4; JEM1 and HAC1; LHS1 and SCJ1; LHS1 and KAR2; LHS1 and SIL1; LHS1 and FKB2; LHS1 and SSA1; LHS1 and SSA2; LHS1 and SSA3; LHS1 and SSA4; LHS1 and SSE1; LHS1 and SSE2; LHS1 and SSB1; LHS1 and SSB2; LHS1 and ECM10; LHS1 and MDJ1; LHS1 and MDJ2; LHS1 and ERV2; LHS1 and EUG1; LHS1 and MPD1; LHS1 and MPD2; LHS1 and EPS1; LHS1 and PDI1; LHS1 and DER1; LHS1 and DER3; LHS1 and HRD3; LHS1 and UBC7; LHS1 and DOA4; LHS1 and HAC1; SCJ1 and KAR2; SCJ1 and SIL1; SCJ1 and FKB2; SCJ1 and SSA1; SCJ1 and SSA2; SCJ1 and SSA3; SCJ1 and SSA4; SCJ1 and SSE1; SCJ1 and SSE2; SCJ1 and SSB1; SCJ1 and SSB2; SCJ1 and ECM10; SCJ1 and MDJ1; SCJ1 and MDJ2; SCJ1 and ERV2; SCJ1 and EUG1; SCJ1 and MPD1; SCJ1 and MPD2; SCJ1 and EPS1; SCJ1 and PDI1; SCJ1 and DER1; SCJ1 and DER3; SCJ1 and HRD3; SCJ1 and UBC7; SCJ1 and DOA4; SCJ1 and HAC1; KAR2 and SIL1; KAR2 and FKB2; KAR2 and SSA1; KAR2 and SSA2; KAR2 and SSA3; KAR2 and SSA4; KAR2 and SSE1; KAR2 and SSE2; KAR2 and SSB1; KAR2 and SSB2; KAR2 and ECM10; KAR2 and MDJ1; KAR2 and MDJ2; KAR2 and ERV2; KAR2 and EUG1; KAR2 and MPD1; KAR2 and MPD2; KAR2 and EPS1; KAR2 and PDI1; KAR2 and DER1; KAR2 and DER3; KAR2 and HRD3; KAR2 and UBC7; KAR2 and DOA4; KAR2 and HAC1; SIL1 and FKB2; SIL1 and SSA1; SIL1 and, SSA2; SIL1 and SSA3; SIL1 and SSA4; SIL1 and SSE1; SIL1 and SSE2; SIL1 and SSB1; Slid and SSB2; SIL1 and ECM10; SIL1 and MDJ1; SIL1 and MDJ2; SIL1 and ERV2; SIL1 and EUG1; SIL1 and MPD1; SIL1 and MPD2; SIL1 and EPS1; SIL1 and PDI1; SIL1 and DER1; SIL1 and DER3; SIL1 and HRD3; SIL1 and UBC7; SIL1 and DOA4; SIL1 and HAC1; FKB2 and SSA1; FKB2 and SSA2; FKB2 and SSA3; FKB2 and SSA4; FKB2 and SSE1; FKB2 and SSE2; FKB2 and SSB1; FKB2 and SSB2; FKB2 and ECM10; FKB2 and MDJ1; FKB2 and MDJ2; FKB2 and ERV2; FKB2 and EUG1; FKB2 and MPD1; FKB2 and MPD2; FKB2 and EPS1; FKB2 and PDI1; FKB2 and DER1; FKB2 and DER3; FKB2 and HRD3; FKB2 and UBC7; FKB2 and DOA4; FKB2 and HAC1; SSA1 and SSA2; SSA1 and SSA3; SSA1 and SSA4; SSA1 and SSE1; SSA1 and SSE2; SSA1 and SSB1; SSA1 and SSB2; SSA1 and ECM10; SSA1 and MDJ1; SSA1 and MDJ2; SSA1 and ERV2; SSA1 and EUG1; SSA1 and MPD1; SSA1 and MPD2; SSA1 and EPS1; SSA1 and PDI1; SSA1 and DER1; SSA1 and DER3; SSA1 and HRD3; SSA1 and UBC7; SSA1 and DOA4; SSA1 and HAC1; SSA2 and SSA3; SSA2 and SSA4; SSA2 and SSE1; SSA2 and SSE2; SSA2 and SSB1; SSA2 and SSB2; SSA2 and ECM10; SSA2 and MDJ1; SSA2 and MDJ2; SSA2 and ERV2; SSA2 and EUG1; SSA2 and MPD1; SSA2 and MPD2; SSA2 and EPS1; SSA2 and PDI1; SSA2 and DER1; SSA2 and DER3; SSA2 and HRD3; SSA2 and UBC7; SSA2 and DOA4; SSA2 and HAC1; SSA3 and SSA4; SSA3 and SSE1; SSA3 and SSE2; SSA3 and SSB1; SSA3 and SSB2; SSA3 and ECM10; SSA3 and MDJ1; SSA3 MDJ2; SSA3 and ERV2; SSA3 and EUG1; SSA3 and MPD1; SSA3 and MPD2; SSA3 and EPS1; SSA3 and PDI1; SSA3 and DER1; SSA3 and DER3; SSA3 and HRD3; SSA3 and UBC7; SSA3 and DOA4; SSA3 and HAC1; SSA4 and SSE1; SSA4 and SSE2; SSA4 and SSB1; SSA4 and SSB2; SSA4 and ECM10; SSA4 and MDJ1; SSA4 and MDJ2; SSA4 and ERV2; SSA4 and EUG1; SSA4 and MPD1; SSA4 and MPD2; SSA4 and EPS1; SSA4 and PDI1; SSA4 and DER1; SSA4 and DER3; SSA4 and HRD3; SSA4 and UBC7; SSA4 and DOA4; SSA4 and HAC1; SSE1 and SSE2; SSE1 and SSB1; SSE1 and SSB2; SSE1 and ECM10; SSE1 and MDJ1; SSE1 and MDJ2; SSE1 and ERV2; SSE1 and EUG1; SSE1 and MPD1; SSE1 and MPD2; SSE1 and EPS1; SSE1 and PDI1; SSE1 and DER1; SSE1 and DER3; SSE1 and HRD3; SSE1 and UBC7; SSE1 and DOA4; SSE1 and HAC1; SSE2 and SSB1; SSE2 and SSB2; SSE2 and ECM10; SSE2 and MDJ1; SSE2 and MDJ2; SSE2 and ERV2; SSE2 and EUG1; SSE2 and MPD1; SSE2 and MPD2; SSE2 and EPS1; SSE2 and PDI1; SSE2 and DER1; SSE2 and DER3; SSE2 and HRD3; SSE2 and UBC7; SSE2 and DOA4; SSE2 and HAC1; SSB1 and SSB2; SSB1 and ECM10; SSB1 and MDJ1; SSB1 and MDJ2; SSB1 and ERV2; SSB1 and EUG1; SSB1 and MPD1; SSB1 and MPD2; SSB1 and EPS1; SSB1 and PDI1; SSB1 and DER1; SSB1 and DER3; SSB1 and HRD3; SSB1 and UBC7; SSB1 and DOA4; SSB1 and HAC1; SSB2 and ECM10; SSB2 and MDJ1; SSB2 and MDJ2; SSB2 and ERV2; SSB2 and EUG1; SSB2 and MPD1; SSB2 and MPD2; SSB2 and EPS1; SSB2 and PDI1; SSB2 and DER1; SSB2 and DER3; SSB2 and HRD3; SSB2 and UBC7; SSB2 and DOA4; SSB2 and HAC1; ECM10 and MDJ1; ECM10 and MDJ2; ECM10 and ERV2; ECM10 and EUG1; ECM10 and MPD1; ECM10 and MPD2; ECM10 and EPS1; ECM10 and PDI1; ECM10 and DER1; ECM10 and DER3; ECM10 and HRD3; ECM10 and UBC7; ECM10 and DOA4; ECM10 and HAC1; MDJ1 and MDJ2; MDJ1 and ERV2; MDJ1 and EUG1; MDJ1 and MPD1; MDJ1 and MPD2; MDJ1 and EPS1; MDJ1 and PDI1; MDJ1 and DER1; MDJ1 and DER3; MDJ1 and HRD3; MDJ1 and UBC7; MDJ1 and DOA4; MDJ1 and HAC1; MDJ2 and ERV2; MDJ2 and EUG1; MDJ2 and MPD1; MDJ2 and MPD2; MDJ2 and EPS1; MDJ2 and PDI1; MDJ2 and DER1; MDJ2 and DER3; MDJ2 and HRD3; MDJ2 and UBC7; MDJ2 and DOA4; MDJ2 and HAC1; ERV2 and EUG1; ERV2 and MPD1; ERV2 and MPD2; ERV2 and EPS1; ERV2 and PDI1; ERV2 and DER1; ERV2 and DER3; ERV2 and HRD3; ERV2 and UBC7; ERV2 and DOA4; ERV2 and HAC1; EUG1 and MPD1; EUG1 and MPD2; EUG1 and EPS1; EUG1 and PDI1; EUG1 and DER1; EUG1 and DER3; EUG1 and HRD3; EUG1 and UBC7; EUG1 and DOA4; EUG1 and HAC1; MPD1 and MPD2; MPD1 and EPS1; MPD1 and PDI1; MPD1 and DER1; MPD1 and DER3; MPD1 and HRD3; MPD1 and UBC7; MPD1 and DOA4; MPD1 and HAC1; MPD2 and EPS1; MPD2 and PDI1; MPD2 and DER1; MPD2 and DER3; MPD2 and HRD3; MPD2 and UBC7; MPD2 and DOA4; MPD2 and HAC1; EPS1 and PDI1; EPS1 and DER1; EPS1 and DER3; EPS1 and HRD3; EPS1 and UBC7; EPS1 and DOA4; EPS1 and HAC1; PDI1 and DER1; PDI1 and DER3; PDI1 and HRD3; PDI1 and UBC7; PDI1 and DOA4; PDI1 and HAC1; DER1 and DER3; DER1 and HRD3; DER1 and UBC7; DER1 and DOA4; DER1 and HAC1; DER3 and HRD3; DER3 and UBC7; DER3 and DOA4; DER3 and HAC1; HRD3 and UBC7; HRD3 and DOA4; HRD3 and HAC1; UBC7 and DOA4; UBC7 and HAC1; or DOA4 and HAC1.

ERV2 in combination with any one of the following combinations: JEM1 and LHS1; JEM1 and SCJ1; JEM1 and KAR2; JEM1 and SIL1; JEM1 and FKB2; JEM1 and SSA1; JEM1 and SSA2; JEM1 and SSA3; JEM1 and SSA4; JEM1 and SSE1; JEM1 and SSE2; JEM1 and SSB1; JEM1 and SSB2; JEM1 and ECM10; JEM1 and MDJ1; JEM1 and MDJ2; JEM1 and ERO1; JEM1 and EUG1; JEM1 and MPD1; JEM1 and MPD2; JEM1 and EPS1; JEM1 and PDI1; JEM1 and DER1; JEM1 and DER3; JEM1 and HRD3; JEM1 and UBC7; JEM1 and DOA4; JEM1 and HAC1; LHS1 and SCJ1; LHS1 and KAR2; LHS1 and SIL1; LHS1 and FKB2; LHS1 and SSA1; LHS1 and SSA2; LHS1 and SSA3; LHS1 and SSA4; LHS1 and SSE1; LHS1 and SSE2; LHS1 and SSB1; LHS1 and SSB2; LHS1 and ECM10; LHS1 and MDJ1; LHS1 and MDJ2; LHS1 and ERO1; LHS1 and EUG1; LHS1 and MPD1; LHS1 and MPD2; LHS1 and EPS1; LHS1 and PDI1; LHS1 and DER1; LHS1 and DER3; LHS1 and HRD3; LHS1 and UBC7; LHS1 and DOA4; LHS1 and HAC1; SCJ1 and KAR2; SCJ1 and SIL1; SCJ1 and FKB2; SCJ1 and SSA1; SCJ1 and SSA2; SCJ1 and SSA3; SCJ1 and SSA4; SCJ1 and SSE1; SCJ1 and SSE2; SCJ1 and SSB1; SCJ1 and SSB2; SCJ1 and ECM10; SCJ1 and MDJ1; SCJ1 and MDJ2; SCJ1 and ERO1; SCJ1 and EUG1; SCJ1 and MPD1; SCJ1 and MPD2; SCJ1 and EPS1; SCJ1 and PDI1; SCJ1 and DER1; SCJ1 and DER3; SCJ1 and HRD3; SCJ1 and UBC7; SCJ1 and DOA4; SCJ1 and HAC1; KAR2 and SIL1; KAR2 and FKB2; KAR2 and SSA1; KAR2 and SSA2; KAR2 and SSA3; KAR2 and SSA4; KAR2 and SSE1; KAR2 and SSE2; KAR2 and SSB1; KAR2 and SSB2; KAR2 and ECM10; KAR2 and MDJ1; KAR2 and MDJ2; KAR2 and ERO1; KAR2 and EUG1; KAR2 and MPD1; KAR2 and MPD2; KAR2 and EPS1; KAR2 and PDI1; KAR2 and DER1; KAR2 and DER3; KAR2 and HRD3; KAR2 and UBC7; KAR2 and DOA4; KAR2 and HAC1; SIL1 and FKB2; SIL1 and SSA1; SIL1 and SSA2; SIL1 and SSA3; SIL1 and SSA4; SIL1 and SSE1; SIL1 and SSE2; SIL1 and SSB1; SIL1 and SSB2; SIL1 and ECM10; SIL1 and MDJ1; SIL1 and MDJ2; SIL1 and ERO1; SIL1 and EUG1; SIL1 and MPD1; SIL1 and MPD2; SIL1 and EPS1; SIL1 and PDI1; SIL1 and DER1; SIL1 and DER3; SIL1 and HRD3; SIL1 and UBC7; SIL1 and DOA4; SIL1 and HAC1; FKB2 and SSA1; FKB2 and SSA2; FKB2 and SSA3; FKB2 and SSA4; FKB2 and SSE1; FKB2 and SSE2; FKB2 and SSB1; FKB2 and SSB2; FKB2 and ECM10; FKB2 and MDJ1; FKB2 and MDJ2; FKB2 and ERO1; FKB2 and EUG1; FKB2 and MPD1; FKB2 and MPD2; FKB2 and EPS1; FKB2 and PDI1; FKB2 and DER1; FKB2 and DER3; FKB2 and HRD3; FKB2 and UBC7; FKB2 and DOA4; FKB2 and HAC1; SSA1 and SSA2; SSA1 and SSA3; SSA1 and SSA4; SSA1 and SSE1; SSA1 and SSE2; SSA1 and SSB1; SSA1 and SSB2; SSA1 and ECM10; SSA1 and MDJ1; SSA1 and MDJ2; SSA1 and ERO1; SSA1 and EUG1; SSA1 and MPD1; SSA1 and MPD2; SSA1 and EPS1; SSA1 and PDI1; SSA1 and DER1; SSA1 and DER3; SSA1 and HRD3; SSA1 and UBC7; SSA1 and DOA4; SSA1 and HAC1; SSA2 and SSA3; SSA2 and SSA4; SSA2 and SSE1; SSA2 and SSE2; SSA2 and SSB1; SSA2 and SSB2; SSA2 and ECM10; SSA2 and MDJ1; SSA2 and MDJ2; SSA2 and ERO1; SSA2 and EUG1; SSA2 and MPD1; SSA2 and MPD2; SSA2 and EPS1; SSA2 and PDI1; SSA2 and DER1; SSA2 and DER3; SSA2 and HRD3; SSA2 and UBC7; SSA2 and DOA4; SSA2 and HAC1; SSA3 and SSA4; SSA3 and SSE1; SSA3 and SSE2; SSA3 and SSB1; SSA3 and SSB2; SSA3 and ECM10; SSA3 and MDJ1; SSA3 and MDJ2; SSA3 and ERO1; SSA3 and EUG1; SSA3 and MPD1; SSA3 and MPD2; SSA3 and EPS1; SSA3 and PDI1; SSA3 and DER1; SSA3 and DER3; SSA3 and HRD3; SSA3 and UBC7; SSA3 and DOA4; SSA3 and HAC1; SSA4 and SSE1; SSA4 and SSE2; SSA4 and SSB1; SSA4 and SSB2; SSA4 and ECM10; SSA4 and MDJ1; SSA4 and MDJ2; SSA4 and ERO1; SSA4 and EUG1; SSA4 and MPD1; SSA4 and MPD2; SSA4 and EPS1; SSA4 and PDI1; SSA4 and DER1; SSA4 and DER3; SSA4 and HRD3; SSA4 and UBC7; SSA4 and DOA4; SSA4 and HAC1; SSE1 and SSE2; SSE1 and SSB1; SSE1 and SSB2; SSE1 and ECM10; SSE1 and MDJ1; SSE1 and MDJ2; SSE1 and ERO1; SSE1 and EUG1; SSE1 and MPD1; SSE1 and MPD2; SSE1 and EPS1; SSE1 and PDI1; SSE1 and DER1; SSE1 and DER3; SSE1 and HRD3; SSE1 and UBC7; SSE1 and DOA4; SSE1 and HAC1; SSE2 and SSB1; SSE2 and SSB2; SSE2 and ECM10; SSE2 and MDJ1; SSE2 and MDJ2; SSE2 and ERO1; SSE2 and EUG1; SSE2 and MPD1; SSE2 and MPD2; SSE2 and EPS1; SSE2 and PDI1; SSE2 and DER1; SSE2 and DER3; SSE2 and HRD3; SSE2 and UBC7; SSE2 and DOA4; SSE2 and HAC1; SSB1 and SSB2; SSB1 and ECM10; SSB1 and MDJ1; SSB1 and MDJ2; SSB1 and ERO1; SSB1 and EUG1; SSB1 and MPD1; SSB1 and MPD2; SSB1 and EPS1; SSB1 and PDI1; SSB1 and DER1; SSB1 and DER3; SSB1 and HRD3; SSB1 and UBC7; SSB1 and DOA4; SSB1 and HAC1; SSB2 and ECM10; SSB2 and MDJ1; SSB2 and MDJ2; SSB2 and ERO1; SSB2 and EUG1; SSB2 and MPD1; SSB2 and MPD2; SSB2 and EPS1; SSB2 and PDI1; SSB2 and DER1; SSB2 and DER3; SSB2 and HRD3; SSB2 and UBC7; SSB2 and DOA4; SSB2 and HAC1; ECM10 and MDJ1; ECM10 and MDJ2; ECM10 and ERO1; ECM10 and EUG1; ECM10 and MPD1; ECM10 and MPD2; ECM10 and EPS1; ECM10 and PDI1; ECM10 and DER1; ECM10 and DER3; ECM10 and HRD3; ECM10 and UBC7; ECM10 and DOA4; ECM10 and MeI; MDJ1 and MDJ2; MDJ1 and ERO1; MDJ1 and EUG1; MDJ1 and MPD1; MDJ1 and MPD2; MDJ1 and EPS1; MDJ1 and PDI1; MDJ1 and DER1; MDJ1 and DER3; MDJ1 and HRD3; MDJ1 and UBC7; MDJ1 and DOA4; MDJ1 and HAC1; MDJ2 and ERO1; MDJ2 and EUG1; MDJ2 and MPD1; MDJ2 and MPD2; MDJ2 and EPS1; MDJ2 and PDI1; MDJ2 and DER1; MDJ2 and DER3; MDJ2 and HRD3; MDJ2 and UBC7; MDJ2 and DOA4; MDJ2 and HAC1; ERO1 and EUG1; ERO1 and MPD1; ERO1 and MPD2; ERO1 and EPS1; ERO1 and PDI1; ERO1 and DER1; ERO1 and DER3; ERO1 and HRD3; ERO1 and UBC7; ERO1 and DOA4; ERO1 and HAC1; EUG1 and MPD1; EUG1 and MPD2; EUG1 and EPS1; EUG1 and PDI1; EUG1 and DER1; EUG1 and DER3; EUG1 and HRD3; EUG1 and UBC7; EUG1 and DOA4; EUG1 and HAC1; MPD1 and MPD2; MPD1 and EPS1; MPD1 and PDI1; MPD1 and DER1; MPD1 and DER3; MPD1 and HRD3; MPD1 and UBC7; MPD1 and DOA4; MPD1 and HAC1; MPD2 and EPS1; MPD2 and PDI1; MPD2 and DER1; MPD2 and DER3; MPD2 and HRD3; MPD2 and UBC7; MPD2 and DOA4; MPD2 and HAC1; EPS1 and PDI1; EPS1 and DER1; EPS1 and DER3; EPS1 and HRD3; EPS1 and UBC7; EPS1 and DOA4; EPS1 and HAC1; PDI1 and DER1; PDI1 and DER3; PDI1 and HRD3; PDI1 and UBC7; PDI1 and DOA4; PDI1 and HAC1; DER1 and DER3; DER1 and HRD3; DER1 and UBC7; DER1 and DOA4; DER1 and HAC1; DER3 and HRD3; DER3 and UBC7; DER3 and DOA4; DER3 and HAC1; HRD3 and UBC7; HRD3 and DOA4; HRD3 and HAC1; UBC7 and DOA4; UBC7 and HAC1; or DOA4 and HAC1.

EUG1 in combination with any one of the following combinations: JEM1 and LHS1; JEM1 and SCJ1; JEM1 and KAR2; JEM1 and SIL1; JEM1 and FKB2; JEM1 and SSA1; JEM1 and SSA2; JEM1 and SSA3; JEM1 and SSA4; JEM1 and SSE1; JEM1 and SSE2; JEM1 and SSB1; JEM1 and SSB2; JEM1 and ECM10; JEM1 and MDJ1; JEM1 and MDJ2; JEM1 and ERO1; JEM1 and ERV2; JEM1 and MPD1; JEM1 and MPD2; JEM1 and EPS1; JEM1 and PDI1; JEM1 and DER1; JEM1 and DER3; JEM1 and HRD3; JEM1 and UBC7; JEM1 and DOA4; JEM1 and HAC1; LHS1 and SCJ1; LHS1 and KAR2; LHS1 and SIL1; LHS1 and FKB2; LHS1 and SSA1; LHS1 and SSA2; LHS1 and SSA3; LHS1 and SSA4; LHS1 and SSE1; LHS1 and SSE2; LHS1 and SSB1; LHS1 and SSB2; LHS1 and ECM10; LHS1 and MDJ1; LHS1 and MDJ2; LHS1 and ERO1; LHS1 and ERV2; LHS1 and MPD1; LHS1 and MPD2; LHS1 and EPS1; LHS1 and PDI1; LHS1 and DER1; LHS1 and DER3; LHS1 and HRD3; LHS1 and UBC7; LHS1 and DOA4; LHS1 and HAC1; SCJ1 and KAR2; SCJ1 and SIL1; SCJ1 and FKB2; SCJ1 and SSA1; SCJ1 and SSA2; SCJ1 and SSA3; SCJ1 and SSA4; SCJ1 and SSE1; SCJ1 and SSE2; SCJ1 and SSB1; SCJ1 and SSB2; SCJ1 and ECM10; SCJ1 and MDJ1; SCJ1 and MDJ2; SCJ1 and ERO1; SCJ1 and ERV2; SCJ1 and MPD1; SCJ1 and MPD2; SCJ1 and EPS1; SCJ1 and PDI1; SCJ1 and DER1; SCJ1 and DER3; SCJ1 and HRD3; SCJ1 and UBC7; SCJ1 and DOA4; SCJ1 and HAC1; KAR2 and SIL1; KAR2 and FKB2; KAR2 and SSA1; KAR2 and SSA2; KAR2 and SSA3; KAR2 and SSA4; KAR2 and SSE1; KAR2 and SSE2; KAR2 and SSB1; KAR2 and SSB2; KAR2 and ECM10; KAR2 and MDJ1; KAR2 and MDJ2; KAR2 and ERO1; KAR2 and ERV2; KAR2 and MPD1; KAR2 and MPD2; KAR2 and EPS1; KAR2 and PDI1; KAR2 and DER1; KAR2 and DER3; KAR2 and HRD3; KAR2 and UBC7; KAR2 and DOA4; KAR2 and HAC1; SIL1 and FKB2; SIL1 and SSA1; SIL1 and SSA2; SIL1 and SSA3; SIL1 and SSA4; SIL1 and SSE1; SIL1 and SSE2; SIL1 and SSB1; SIL1 and SSB2; SIL1 and ECM10; SIL1 and MDJ1; SIL1 and MDJ2; SIL1 and ERO1; SIL1 and ERV2; SIL1 and MPD1; SIL1 and MPD2; SIL1 and EPS1; SIL1 and PDI1; SIL1 and DER1; SIL1 and DER3; SIL1 and HRD3; SIL1 and UBC7; SIL1 and DOA4; SIL1 and HAC1; FKB2 and SSA1; FKB2 and SSA2; FKB2 and SSA3; FKB2 and SSA4; FKB2 and SSE1; FKB2 and SSE2; FKB2 and SSB1; FKB2 and SSB2; FKB2 and ECM10; FKB2 and MDJ1; FKB2 and MDJ2; FKB2 and ERO1; FKB2 and ERV2; FKB2 and MPD1; FKB2 and MPD2; FKB2 and EPS1; FKB2 and PDI1; FKB2 and DER1; FKB2 and DER3; FKB2 and HRD3; FKB2 and UBC7; FKB2 and DOA4; FKB2 and HAC1; SSA1 and SSA2; SSA1 and SSA3; SSA1 and SSA4; SSA1 and SSE1; SSA1 and SSE2; SSA1 and SSB1; SSA1 and SSB2; SSA1 and ECM10; SSA1 and MDJ1; SSA1 and MDJ2; SSA1 and ERO1; SSA1 and ERV2; SSA1 and MPD1; SSA1 and MPD2; SSA1 and EPS1; SSA1 and PDI1; SSA1 and DER1; SSA1 and DER3; SSA1 and HRD3; SSA1 and UBC7; SSA1 and DOA4; SSA1 and HAC1; SSA2 and SSA3; SSA2 and SSA4; SSA2 and SSE1; SSA2 and SSE2; SSA2 and SSB1; SSA2 and SSB2; SSA2 and ECM10; SSA2 and MDJ1; SSA2 and MDJ2; SSA2 and ERO1; SSA2 and ERV2; SSA2 and MPD1; SSA2 and MPD2; SSA2 and EPS1; SSA2 and PDI1; SSA2 and DER1; SSA2 and DER3; SSA2 and HRD3; SSA2 and UBC7; SSA2 and DOA4; SSA2 and HAC1; SSA3 and SSA4; SSA3 and SSE1; SSA3 and SSE2; SSA3 and SSB1; SSA3 and SSB2; SSA3 and ECM10; SSA3 and MDJ1; SSA3 and MDJ2; SSA3 and ERO1; SSA3 and ERV2; SSA3 and MPD1; SSA3 and MPD2; SSA3 and EPS1; SSA3 and PDI1; SSA3 and DER1; SSA3 and DER3; SSA3 and HRD3; SSA3 and UBC7; SSA3 and DOA4; SSA3 and HAC1; SSA4 and SSE1; SSA4 and SSE2; SSA4 and SSB1; SSA4 and SSB2; SSA4 and ECM10; SSA4 and MDJ1; SSA4 and MDJ2; SSA4 and ERO1; SSA4 and ERV2; SSA4 and MPD1; SSA4 and MPD2; SSA4 and EPS1; SSA4 and PDI1; SSA4 and DER1; SSA4 and DER3; SSA4 and HRD3; SSA4 and UBC7; SSA4 and DOA4; SSA4 and HAC1; SSE1 and SSE2; SSE1 and SSB1; SSE1 and SSB2; SSE1 and ECM10; SSE1 and MDJ1; SSE1 and MDJ2; SSE1 and ERO1; SSE1 and ERV2; SSE1 and MPD1; SSE1 and MPD2; SSE1 and EPS1; SSE1 and PDI1; SSE1 and DER1; SSE1 and DER3; SSE1 and HRD3; SSE1 and UBC7; SSE1 and DOA4; SSE1 and HAC1; SSE2 and SSB1; SSE2 and SSB2; SSE2 and ECM10; SSE2 and MDJ1; SSE2 and MDJ2; SSE2 and ERO1; SSE2 and ERV2; SSE2 and MPD1; SSE2 and MPD2; SSE2 and EPS1; SSE2 and PDI1; SSE2 and DER1; SSE2 and DER3; SSE2 and HRD3; SSE2 and UBC7; SSE2 and DOA4; SSE2 and HAC1; SSB1 and SSB2; SSB1 and ECM10; SSB1 and MDJ1; SSB1 and MDJ2; SSB1 and ERO1; SSB1 and ERV2; SSB1 and MPD1; SSB1 and MPD2; SSB1 and EPS1; SSB1 and PDI1; SSB1 and DER1; SSB1 and DER3; SSB1 and HRD3; SSB1 and UBC7; SSB1 and DOA4; SSB1 and HAC1; SSB2 and ECM10; SSB2 and MDJ1; SSB2 and MDJ2; SSB2 and ERO1; SSB2 and ERV2; SSB2 and MPD1; SSB2 and MPD2; SSB2 and EPS1; SSB2 and PDI1; SSB2 and DER1; SSB2 and DER3; SSB2 and HRD3; SSB2 and UBC7; SSB2 and DOA4; SSB2 and HAC1; ECM10 and MDJ1; ECM10 and MDJ2; ECM10 and ERO1; ECM10 and ERV2; ECM10 and MPD1; ECM10 and MPD2; ECM10 and EPS1; ECM10 and PDI1; ECM10 and DER1; ECM10 and DER3; ECM10 and HRD3; ECM10 and UBC7; ECM10 and DOA4; ECM10 and HAC1; MDJ1 and MDJ2; MDJ1 and ERO1; MDJ1 and ERV2; MDJ1 and MPD1; MDJ1 and MPD2; MDJ1 and EPS1; MDJ1 and PDI1; MDJ1 and DER1; MDJ1 and DER3; MDJ1 and HRD3; MDJ1 and UBC7; MDJ1 and DOA4; MDJ1 and HAC1; MDJ2 and ERO1; MDJ2 and ERV2; MDJ2 and MPD1; MDJ2 and MPD2; MDJ2 and EPS1; MDJ2 and PDI1; MDJ2 and DER1; MDJ2 and DER3; MDJ2 and HRD3; MDJ2 and UBC7; MDJ2 and DOA4; MDJ2 and HAC1; ERO1 and ERV2; ERO1 and MPD1; ERO1 and MPD2; ERO1 and EPS1; ERO1 and PDI1; ERO1 and DER1; ERO1 and DER3; ERO1 and HRD3; ERO1 and UBC7; ERO1 and DOA4; ERO1 and HAC1; ERV2 and MPD1; ERV2 and MPD2; ERV2 and EPS1; ERV2 and PDI1; ERV2 and DER1; ERV2 and DER3; ERV2 and HRD3; ERV2 and UBC7; ERV2 and DOA4; ERV2 and HAC1; MPD1 and MPD2; MPD1 and EPS1; MPD1 and PDI1; MPD1 and DER1; MPD1 and DER3; MPD1 and HRD3; MPD1 and UBC7; MPD1 and DOA4; MPD1 and HAC1; MPD2 and EPS1; MPD2 and PDI1; MPD2 and DER1; MPD2 and DER3; MPD2 and HRD3; MPD2 and UBC7; MPD2 and DOA4; MPD2 and HAC1; EPS1 and PDI1; EPS1 and DER1; EPS1 and DER3; EPS1 and HRD3; EPS1 and UBC7; EPS1 and DOA4; EPS1 and HAC1; PDI1 and DER1; PDI1 and DER3; PDI1 and HRD3; PDI1 and UBC7; PDI1 and DOA4; PDI1 and HAC1; DER1 and DER3; DER1 and HRD3; DER1 and UBC7; DER1 and DOA4; DER1 and HAC1; DER3 and HRD3; DER3 and UBC7; DER3 and DOA4; DER3 and HAC1; HRD3 and UBC7; HRD3 and DOA4; HRD3 and HAC1; UBC7 and DOA4; UBC7 and HAC1; or DOA4 and HAC1.

MPD1 in combination with any one of the following combinations: JEM1 and LHS1; JEM1 and SCJ1; JEM1 and KAR2; JEM1 and SIL1; JEM1 and FKB2; JEM1 and SSA1; JEM1 and SSA2; JEM1 and SSA3; JEM1 and SSA4; JEM1 and SSE1; JEM1 and SSE2; JEM1 and SSB1; JEM1 and SSB2; JEM1 and ECM10; JEM1 and MDJ1; JEM1 and MDJ2; JEM1 and ERO1; JEW and ERV2; JEM1 and EUG1; JEM1 and MPD2; JEM1 and EPS1; JEM1 and PDI1; JEM1 and DER1; JEM1 and DER3; JEM1 and HRD3; JEM1 and UBC7; JEM1 and DOA4; JEM1 and HAC1; LHS1 and SCJ1; LHS1 and KAR2; LHS1 and SIL1 LHS1 and FKB2; LHS1 and SSA1; LHS1 and SSA2; LHS1 and SSA3; LHS1 and SSA4; LHS1 and SSE1; LHS1 and SSE2; LHS1 and SSB1; LHS1 and SSB2; LHS1 and ECM10; LHS1 and MDJ1; LHS1 and MDJ2; LHS1 and ERO1; LHS1 and ERV2; LHS1 and EUG1; LHS1 and MPD2; LHS1 and EPS1; LHS1 and PDI1; LHS1 and DER1; LHS1 and DER3; LHS1 and HRD3; LHS1 and UBC7; LHS1 and DOA4; LHS1 and HAC1; SCJ1 and KAR2; SCJ1 and SIL1; SCJ1 and FKB2; SCJ1 and SSA1; SCJ1 and SSA2; SCJ1 and SSA3; SCJ1 and SSA4; SCJ1 and SSE1; SCJ1 and SSE2; SCJ1 and SSB1; SCJ1 and SSB2; SCJ1 and ECM10; SCJ1 and MDJ1; SCJ1 and MDJ2; SCJ1 and ERO1; SCJ1 and ERV2; SCJ1 and EUG1; SCJ1 and MPD2; SCJ1 and EPS1; SCJ1 and PDI1; SCJ1 and DER1; SCJ1 and DER3; SCJ1 and HRD3; SCJ1 and UBC7; SCJ1 and DOA4; SCJ1 and HAC1; KAR2 and SIL1; KAR2 and FKB2; KAR2 and SSA1; KAR2 and SSA2; KAR2 and SSA3; KAR2 and SSA4; KAR2 and SSE1; KAR2 and SSE2; KAR2 and SSB1; KAR2 and SSB2; KAR2 and ECM10; KAR2 and MDJ1; KAR2 and MDJ2; KAR2 and ERO1; KAR2 and ERV2; KAR2 and EUG1; KAR2 and MPD2; KAR2 and EPS1; KAR2 and PDI1; KAR2 and DER1; KAR2 and DER3; KAR2 and HRD3; KAR2 and UBC7; KAR2 and DOA4; KAR2 and HAC1; SIL1 and FKB2; SIL1 and SSA1; SIL1 and SSA2; SIL1 and SSA3; SIL1 and SSA4; SIL1 and SSE1; SIL1 and SSE2; SIL1 and SSB1; SIL1 and SSB2; SIL1 and ECM10; SIL1 and MDJ1; SIL1 and MDJ2; SIL1 and ERO1; SIL1 and ERV2; SIL1 and EUG1; SIL1 and MPD2; SIL1 and EPS1; SIL1 and PDI1; SIL1 and DER1; SEM and DER3; SIL1 and HRD3; SIL1 and UBC7; SIL1 and DOA4; SIL1 and HAC1; FKB2 and SSA1; FKB2 and SSA2; FKB2 and SSA3; FKB2 and SSA4; FKB2 and SSE1; FKB2 and SSE2; FKB2 and SSB1; FKB2 and SSB2; FKB2 and ECM10; FKB2 and MDJ1; FKB2 and MDJ2; FKB2 and ERO1; FKB2 and ERV2; FKB2 and EUG1; FKB2 and MPD2; FKB2 and EPS1; FKB2 and PDI1; FKB2 and DER1; FKB2 and DER3; FKB2 and HRD3; FKB2 and UBC7; FKB2 and DOA4; FKB2 and HAC1; SSA1 and SSA2; SSA1 and SSA3; SSA1 and SSA4; SSA1 and SSE1; SSA1 and SSE2; SSA1 and SSB1; SSA1 and SSB2; SSA1 and ECM10; SSA1 and MDJ1; SSA1 and MDJ2; SSA1 and ERO1; SSA1 and ERV2; SSA1 and EUG1; SSA1 and MPD2; SSA1 and EPS1; SSA1 and PDI1; SSA1 and DER1; SSA1 and DER3; SSA1 and HRD3; SSA1 and UBC7; SSA1 and DOA4; SSA1 and HAC1; SSA2 and SSA3; SSA2 and SSA4; SSA2 and SSE1; SSA2 and SSE2; SSA2 and SSB1; SSA2 and SSB2; SSA2 and ECM10; SSA2 and MDJ1; SSA2 and MDJ2; SSA2 and ERO1; SSA2 and ERV2; SSA2 and EUG1; SSA2 and MPD2; SSA2 and EPS1; SSA2 and PDI1; SSA2 and DER1; SSA2 and DER3; SSA2 and HRD3; SSA2 and UBC7; SSA2 and DOA4; SSA2 and HAC1; SSA3 and SSA4; SSA3 and SSE1; SSA3 and SSE2; SSA3 and SSB1; SSA3 and SSB2; SSA3 and ECM10; SSA3 and MDJ1; SSA3 and MDJ2; SSA3 and ERO1; SSA3 and ERV2; SSA3 and EUG1; SSA3 and MPD2; SSA3 and EPS1; SSA3 and PDI1; SSA3 and DER1; SSA3 and DER3; SSA3 and HRD3; SSA3 and UBC7; SSA3 and DOA4; SSA3 and HAC1; SSA4 and SSE1; SSA4 and SSE2; SSA4 and SSB1; SSA4 and SSB2; SSA4 and ECM10; SSA4 and MDJ1; SSA4 and MDJ2; SSA4 and ERO1; SSA4 and ERV2; SSA4 and EUG1; SSA4 and MPD2; SSA4 and EPS1; SSA4 and PDI1; SSA4 and DER1; SSA4 and DER3; SSA4 and HRD3; SSA4 and UBC7; SSA4 and DOA4; SSA4 and HAC1; SSE1 and SSE2; SSE1 and SSB1; SSE1 and SSB2; SSE1 and ECM10; SSE1 and MDJ1; SSE1 and MDJ2; SSE1 and ERO1; SSE1 and ERV2; SSE1 and EUG1; SSE1 and MPD2; SSE1 and EPS1; SSE1 and PDI1; SSE1 and DER1; SSE1 and DER3; SSE1 and HRD3; SSE1 and UBC7; SSE1 and DOA4; SSE1 and HAC1; SSE2 and SSB1; SSE2 and SSB2; SSE2 and ECM10; SSE2 and MDJ1; SSE2 and MDJ2; SSE2 and ERO1; SSE2 and ERV2; SSE2 and EUG1; SSE2 and MPD2; SSE2 and EPS1; SSE2 and PDI1; SSE2 and DER1; SSE2 and DER3; SSE2 and HRD3; SSE2 and UBC7; SSE2 and DOA4; SSE2 and HAC1; SSB1 and SSB2; SSB1 and ECM10; SSB1 and MDJ1; SSB1 and MDJ2; SSB1 and ERO1; SSB1 and ERV2; SSB1 and EUG1; SSB1 and MPD2; SSB1 and EPS1; SSB1 and PDI1; SSB1 and DER1; SSB1 and DER3; SSB1 and HRD3; SSB1 and UBC7; SSB1 and DOA4; SSB1 and HAC1; SSB2 and ECM10; SSB2 and MDJ1; SSB2 and MDJ2; SSB2 and ERO1; SSB2 and ERV2; SSB2 and EUG1; SSB2 and MPD2; SSB2 and EPS1; SSB2 and PDI1; SSB2 and DER1; SSB2 and DER3; SSB2 and HRD3; SSB2 and UBC7; SSB2 and DOA4; SSB2 and HAC1; ECM10 and MDJ1; ECM10 and MDJ2; ECM10 and ERO1; ECM10 and ERV2; ECM10 and EUG1; ECM10 and MPD2; ECM10 and EPS1; ECM10 and PDI1; ECM10 and DER1; ECM10 and DER3; ECM10 and HRD3; ECM10 and UBC7; ECM10 and DOA4; ECM10 and HAC1; MDJ1 and MDJ2; MDJ1 and ERO1; MDJ1 and ERV2; MDJ1 and EUG1; MDJ1 and MPD2; MDJ1 and EPS1; MDJ1 and PDI1; MDJ1 and DER1; MDJ1 and DER3; MDJ1 and HRD3; MDJ1 and UBC7; MDJ1 and DOA4; MDJ1 and HAC1; MDJ2 and ERO1; MDJ2 and ERV2; MDJ2 and EUG1; MDJ2 and MPD2; MDJ2 and EPS1; MDJ2 and PDI1; MDJ2 and DER1; MDJ2 and DER3; MDJ2 and HRD3; MDJ2 and UBC7; MDJ2 and DOA4; MDJ2 and HAC1; ERO1 and ERV2; ERO1 and EUG1; ERO1 and MPD2; ERO1 and EPS1; ERO1 and PDI1; ERO1 and DER1; ERO1 and DER3; ERO1 and HRD3; ERO1 and UBC7; ERO1 and DOA4; ERO1 and HAC1; ERV2 and EUG1; ERV2 and MPD2; ERV2 and EPS1; ERV2 and PDI1; ERV2 and DER1; ERV2 and DER3; ERV2 and HRD3; ERV2 and UBC7; ERV2 and DOA4; ERV2 and HAC1; EUG1 and MPD2; EUG1 and EPS1; EUG1 and PDI1; EUG1 and DER1; EUG1 and DER3; EUG1 and HRD3; EUG1 and UBC7; EUG1 and DOA4; EUG1 and HAC1; MPD2 and EPS1; MPD2 and PDI1; MPD2 and DER1; MPD2 and DER3; MPD2 and HRD3; MPD2 and UBC7; MPD2 and DOA4; MPD2 and HAC1; EPS1 and PDI1; EPS1 and DER1; EPS1 and DER3; EPS1 and HRD3; EPS1 and UBC7; EPS1 and DOA4; EPS1 and HAC1; PDI1 and DER1; PDI1 and DER3; PDI1 and HRD3; PDI1 and UBC7; PDI1 and DOA4; PDI1 and HAC1; DER1 and DER3; DER1 and HRD3; DER1 and UBC7; DER1 and DOA4; DER1 and HAC1; DER3 and HRD3; DER3 and UBC7; DER3 and DOA4; DER3 and HAC1; HRD3 and UBC7; HRD3 and DOA4; HRD3 and HAC1; UBC7 and DOA4; UBC7 and HAC1; or DOA4 and HAC1.

MPD2 in combination with any one of the following combinations: JEM1 and LHS1; JEM1 and SCJ1; JEM1 and KAR2; JEM1 and SIL1; JEM1 and FKB2; JEM1 and SSA1; JEM1 and SSA2; JEM1 and SSA3; JEM1 and SSA4; JEM1 and SSE1; JEM1 and SSE2; JEM1 and SSB1; JEM1 and SSB2; JEM1 and ECM10; JEM1 and MDJ1; JEM1 and MDJ2; JEM1 and ERO1; JEM1 and ERV2; JEM1 and EUG1; JEM1 and MPD1; JEM1 and EPS1; JEM1 and PDI1; JEM1 and DER1; JEM1 and DER3; JEM1 and HRD3; JEM1 and UBC7; JEM1 and DOA4; JEM1 and HAC1; LHS1 and SCJ1; LHS1 and KAR2; LHS1 and MIA; LHS1 and FKB2; LHS1 and SSA1; LHS1 and SSA2; LHS1 and SSA3; LHS1 and SSA4; LHS1 and SSE1; LHS1 and SSE2; LHS1 and SSB1; LHS1 and SSB2; LHS1 and ECM10; LHS1 and MDJ1; LHS1 and MDJ2; LHS1 and ERO1; LHS1 and ERV2; LHS1 and EUG1; LHS1 and MPD1; LHS1 and EPS1; LHS1 and PDI1; LHS1 and DER1; LHS1 and DER3; LHS1 and HRD3; LHS1 and UBC7; LHS1 and DOA4; LHS1 and HAC1; SCJ1 and KAR2; SCJ1 and SIL1; SCJ1 and FKB2; SCJ1 and SSA1; SCJ1 and SSA2; SCJ1 and SSA3; SCJ1 and SSA4; SCJ1 and SSE1; SCJ1 and SSE2; SCJ1 and SSB1; SCJ1 and SSB2; SCJ1 and ECM10; SCJ1 and MDJ1; SCJ1 and MDJ2; SCJ1 and ERO1; SCJ1 and ERV2; SCJ1 and EUG1; SCJ1 and MPD1; SCJ1 and EPS1; SCJ1 and PDI1; SCJ1 and DER1; SCJ1 and DER3; SCJ1 and HRD3; SCJ1 and UBC7; SCJ1 and DOA4; SCJ1 and HAC1; KAR2 and SIL1; KAR2 and FKB2; KAR2 and SSA1; KAR2 and SSA2; KAR2 and SSA3; KAR2 and SSA4; KAR2 and SSE1; KAR2 and SSE2; KAR2 and SSB1; KAR2 and SSB2; KAR2 and ECM10; KAR2 and MDJ1; KAR2 and MDJ2; KAR2 and ERO1; KAR2 and ERV2; KAR2 and EUG1; KAR2 and MPD1; KAR2 and EPS1; KAR2 and PDI1; KAR2 and DER1; KAR2 and DER3; KAR2 and HRD3; KAR2 and UBC7; KAR2 and DOA4; KAR2 and HAC1; SIL1 and FKB2; SIL1 and SSA1; SIL1 and SSA2; SIL1 and SSA3; SIL1 and SSA4; SIL1 and SSE1; SIL1 and SSE2; SIL1 and SSB1; SIL1 and SSB2; SIL1 and ECM10; SIL1 and MDJ1; SIL1 and MDJ2; SIL1 and ERO1; SIL1 and ERV2; SIL1 and EUG1; SIL1 and MPD1; SIL1 and EPS1; SIL1 and PDI1; SIL1 and DER1; SIL1 and DER3; SIL1 and HRD3; SIL1 and UBC7; SIL1 and DOA4; SIL1 and HAC1; FKB2 and SSA1; FKB2 and SSA2; FKB2 and SSA3; FKB2 and SSA4; FKB2 and SSE1; FKB2 and SSE2; FKB2 and SSB1; FKB2 and SSB2; FKB2 and ECM10; FKB2 and MDJ1; FKB2 and MDJ2; FKB2 and ERO1; FKB2 and ERV2; FKB2 and EUG1; FKB2 and MPD1; FKB2 and EPS1; FKB2 and PDI1; FKB2 and DER1; FKB2 and DER3; FKB2 and HRD3; FKB2 and UBC7; FKB2 and DOA4; FKB2 and HAC1; SSA1 and SSA2; SSA1 and SSA3; SSA1 and SSA4; SSA1 and SSE1; SSA1 and SSE2; SSA1 and SSB1; SSA1 and SSB2; SSA1 and ECM10; SSA1 and MDJ1; SSA1 and MDJ2; SSA1 and ERO1; SSA1 and ERV2; SSA1 and EUG1; SSA1 and MPD1; SSA1 and EPS1; SSA1 and PDI1; SSA1 and DER1; SSA1 and DER3; SSA1 and HRD3; SSA1 and UBC7; SSA1 and DOA4; SSA1 and HAC1; SSA2 and SSA3; SSA2 and SSA4; SSA2 and SSE1; SSA2 and SSE2; SSA2 and SSB1; SSA2 and SSB2; SSA2 and ECM10; SSA2 and MDJ1; SSA2 and MDJ2; SSA2 and ERO1; SSA2 and ERV2; SSA2 and EUG1; SSA2 and MPD1; SSA2 and EPS1; SSA2 and PDI1; SSA2 and DER1; SSA2 and DER3; SSA2 and HRD3; SSA2 and UBC7; SSA2 and DOA4; SSA2 and HAC1; SSA3 and SSA4; SSA3 and SSE1; SSA3 and SSE2; SSA3 and SSB1; SSA3 and SSB2; SSA3 and ECM10; SSA3 and MDJ1; SSA3 and MDJ2; SSA3 and ERO1; SSA3 and ERV2; SSA3 and EUG1; SSA3 and MPD1; SSA3 and EPS1; SSA3 and PDI1; SSA3 and DER1; SSA3 and DER3; SSA3 and HRD3; SSA3 and UBC7; SSA3 and DOA4; SSA3 and HAC1; SSA4 and SSE1; SSA4 and SSE2; SSA4 and SSB1; SSA4 and SSB2; SSA4 and ECM10; SSA4 and MDJ1; SSA4 and MDJ2; SSA4 and ERO1; SSA4 and ERV2; SSA4 and EUG1; SSA4 and MPD1; SSA4 and EPS1; SSA4 and PDI1; SSA4 and DER1; SSA4 and DER3; SSA4 and HRD3; SSA4 and UBC7; SSA4 and DOA4; SSA4 and HAC1; SSE1 and SSE2; SSE1 and SSB1; SSE1 and SSB2; SSE1 and ECM10; SSE1 and MDJ1; SSE1 and MDJ2; SSE1 and ERO1; SSE1 and ERV2; SSE1 and EUG1; SSE1 and MPD1; SSE1 and EPS1; SSE1 and PDI1; SSE1 and DER1; SSE1 and DER3; SSE1 and HRD3; SSE1 and UBC7; SSE1 and DOA4; SSE1 and HAC1; SSE2 and SSB1; SSE2 and SSB2; SSE2 and ECM10; SSE2 and MDJ1; SSE2 and MDJ2; SSE2 and ERO1; SSE2 and ERV2; SSE2 and EUG1; SSE2 and MPD1; SSE2 and EPS1; SSE2 and PDI1; SSE2 and DER1; SSE2 and DER3; SSE2 and HRD3; SSE2 and UBC7; SSE2 and DOA4; SSE2 and HAC1; SSB1 and SSB2; SSB1 and ECM10; SSB1 and MDJ1; SSB1 and MDJ2; SSB1 and ERO1; SSB1 and ERV2; SSB1 and EUG1; SSB1 and MPD1; SSB1 and EPS1; SSB1 and PDI1; SSB1 and DER1; SSB1 and DER3; SSB1 and HRD3; SSB1 and UBC7; SSB1 and DOA4; SSB1 and HAC1; SSB2 and ECM10; SSB2 and MDJ1; SSB2 and MDJ2; SSB2 and ERO1; SSB2 and ERV2; SSB2 and EUG1; SSB2 and MPD1; SSB2 and EPS1; SSB2 and PDI1; SSB2 and DER1; SSB2 and DER3; SSB2 and HRD3; SSB2 and UBC7; SSB2 and DOA4; SSB2 and HAC1; ECM10 and MDJ1; ECM10 and MDJ2; ECM10 and ERO1; ECM10 and ERV2; ECM10 and EUG1; ECM10 and MPD1; ECM10 and EPS1; ECM10 and PDI1; ECM10 and DER1; ECM10 and DER3; ECM10 and HRD3; ECM10 and UBC7; ECM10 and DOA4; ECM10 and HAC1; MDJ1 and MDJ2; MDJ1 and ERO1; MDJ1 and ERV2; MDJ1 and EUG1; MDJ1 and MPD1; MDJ1 and EPS1; MDJ1 and PDI1; MDJ1 and DER1; MDJ1 and DER3; MDJ1 and HRD3; MDJ1 and UBC7; MDJ1 and DOA4; MDJ1 and HAC1; MDJ2 and ERO1; MDJ2 and ERV2; MDJ2 and EUG1; MDJ2 and MPD1; MDJ2 and EPS1; MDJ2 and PDI1; MDJ2 and DER1; MDJ2 and DER3; MDJ2 and HRD3; MDJ2 and UBC7; MDJ2 and DOA4; MDJ2 and HAC1; ERO1 and ERV2; ERO1 and EUG1; ERO1 and MPD1; ERO1 and EPS1; ERO1 and PDI1; ERO1 and DER1; ERO1 and DER3; ERO1 and HRD3; ERO1 and UBC7; ERO1 and DOA4; ERO1 and HAC1; ERV2 and EUG1; ERV2 and MPD1; ERV2 and EPS1; ERV2 and PDI1; ERV2 and DER1; ERV2 and DER3; ERV2 and HRD3; ERV2 and UBC7; ERV2 and DOA4; ERV2 and HAC1; EUG1 and MPD1; EUG1 and EPS1; EUG1 and PDI1; EUG1 and DER1; EUG1 and DER3; EUG1 and HRD3; EUG1 and UBC7; EUG1 and DOA4; EUG1 and HAC1; MPD1 and EPS1; MPD1 and PDI1; MPD1 and DER1; MPD1 and DER3; MPD1 and HRD3; MPD1 and UBC7; MPD1 and DOA4; MPD1 and HAC1; EPS1 and PDI1; EPS1 and DER1; EPS1 and DER3; EPS1 and HRD3; EPS1 and UBC7; EPS1 and DOA4; EPS1 and HAC1; PDI1 and DER1; PDI1 and DER3; PDI1 and HRD3; PDI1 and UBC7; PDI1 and DOA4; PDI1 and HAC1; DER1 and DER3; DER1 and HRD3; DER1 and UBC7; DER1 and DOA4; DER1 and HAC1; DER3 and HRD3; DER3 and UBC7; DER3 and DOA4; DER3 and HAC1; HRD3 and UBC7; HRD3 and DOA4; HRD3 and HAC1; UBC7 and DOA4; UBC7 and HAC1; or DOA4 and HAC1.

EPS1 in combination with any one of the following combinations: JEM1 and LHS1; JEM1 and SCJ1; JEM1 and KAR2; JEM1 and SIL1; JEM1 and FKB2; JEM1 and SSA1; JEM1 and SSA2; JEM1 and SSA3; JEM1 and SSA4; JEM1 and SSE1; JEM1 and SSE2; JEM1 and SSB1; JEM1 and SSB2; JEM1 and ECM10; JEM1 and MDJ1; JEM1 and MDJ2; JEM1 and ERO1; JEM1 and ERV2; JEM1 and EUG1; JEM1 and MPD1; JEM1 and MPD2; JEM1 and PDI1; JEM1 and DER1; JEM1 and DER3; JEM1 and HRD3; JEM1 and UBC7; JEM1 and DOA4; JEM1 and HAC1; LHS1 and SCJ1; LHS1 and KAR2; LHS1 and SIL1; LHS1 and FKB2; LHS1 and SSA1; LHS1 and SSA2; LHS1 and SSA3; LHS1 and SSA4; LHS1 and SSE1; LHS1 and SSE2; LHS1 and SSB1; LHS1 and SSB2; LHS1 and ECM10; LHS1 and MDJ1; LHS1 and MDJ2; LHS1 and ERO1; LHS1 and ERV2; LHS1 and EUG1; LHS1 and MPD1; LHS1 and MPD2; LHS1 and PDI1; LHS1 and DER1; LHS1 and DER3; LHS1 and HRD3; LHS1 and UBC7; LHS1 and DOA4; LHS1 and HAC1; SCJ1 and KAR2; SCJ1 and SIL1; SCJ1 and FKB2; SCJ1 and SSA1; SCJ1 and SSA2; SCJ1 and SSA3; SCJ1 and SSA4; SCJ1 and SSE1; SCJ1 and SSE2; SCJ1 and SSB1; SCJ1 and SSB2; SCJ1 and ECM10; SCJ1 and MDJ1; SCJ1 and MDJ2; SCJ1 and ERO1; SCJ1 and ERV2; SCJ1 and EUG1; SCJ1 and MPD1; SCJ1 and MPD2; SCJ1 and PDI1; SCJ1 and DER1; SCJ1 and DER3; SCJ1 and HRD3; SCJ1 and UBC7; SCJ1 and DOA4; SCJ1 and HAC1; KAR2 and SIL1; KAR2 and FKB2; KAR2 and SSA1; KAR2 and SSA2; KAR2 and SSA3; KAR2 and SSA4; KAR2 and SSE1; KAR2 and SSE2; KAR2 and SSB1; KAR2 and SSB2; KAR2 and ECM10; KAR2 and MDJ1; KAR2 and MDJ2; KAR2 and ERO1; KAR2 and ERV2; KAR2 and EUG1; KAR2 and MPD1; KAR2 and MPD2; KAR2 and PDI1; KAR2 and DER1; KAR2 and DER3; KAR2 and HRD3; KAR2 and UBC7; KAR2 and DOA4; KAR2 and HAC1; SIL1 and FKB2; SIL1 and SSA1; SIL1 and SSA2; SIL1 and SSA3; SIL1 and SSA4; SIL1 and SSE1; SIL1 and SSE2; SIL1 and SSB1; SIL1 and SSB2; SIL1 and ECM10; SIL1 and MDJ1; SIL1 and MDJ2; SIL1 and ERO1; SIL1 and ERV2; SIL1 and EUG1; SIL1 and MPD1; SIL1 and MPD2; SIL1 and PDI1; SIL1 and DER1; SIL1 and DER3; SIL1 and HRD3; SIL1 and UBC7; SIL1 and DOA4; SIL1 and HAC1; FKB2 and SSA1; FKB2 and SSA2; FKB2 and SSA3; FKB2 and SSA4; FKB2 and SSE1; FKB2 and SSE2; FKB2 and SSB1; FKB2 and SSB2; FKB2 and ECM10; FKB2 and MDJ1; FKB2 and MDJ2; FKB2 and ERO1; FKB2 and ERV2; FKB2 and EUG1; FKB2 and MPD1; FKB2 and MPD2; FKB2 and PDI1; FKB2 and DER1; FKB2 and DER3; FKB2 and HRD3; FKB2 and UBC7; FKB2 and DOA4; FKB2 and HAC1; SSA1 and SSA2; SSA1 and SSA3; SSA1 and SSA4; SSA1 and SSE1; SSA1 and SSE2; SSA1 and SSB1; SSA1 and SSB2; SSA1 and ECM10; SSA1 and MDJ1; SSA1 and MDJ2; SSA1 and ERO1; SSA1 and ERV2; SSA1 and EUG1; SSA1 and MPD1; SSA1 and MPD2; SSA1 and PDI1; SSA1 and DER1; SSA1 and DER3; SSA1 and HRD3; SSA1 and UBC7; SSA1 and DOA4; SSA1 and HAC1; SSA2 and SSA3; SSA2 and SSA4; SSA2 and SSE1; SSA2 and SSE2; SSA2 and SSB1; SSA2 and SSB2; SSA2 and ECM10; SSA2 and MDJ1; SSA2 and MDJ2; SSA2 and ERO1; SSA2 and ERV2; SSA2 and EUG1; SSA2 and MPD1; SSA2 and MPD2; SSA2 and PDI1; SSA2 and DER1; SSA2 and DER3; SSA2 and HRD3; SSA2 and UBC7; SSA2 and DOA4; SSA2 and HAC1; SSA3 and SSA4; SSA3 and SSE1; SSA3 and SSE2; SSA3 and SSB1; SSA3 and SSB2; SSA3 and ECM10; SSA3 and MDJ1; SSA3 and MDJ2; SSA3 and ERO1; SSA3 and ERV2; SSA3 and EUG1; SSA3 and MPD1; SSA3 and MPD2; SSA3 and PDI1; SSA3 and DER1; SSA3 and DER3; SSA3 and HRD3; SSA3 and UBC7; SSA3 and DOA4; SSA3 and HAC1; SSA4 and SSE1; SSA4 and SSE2; SSA4 and SSB1; SSA4 and SSB2; SSA4 and ECM10; SSA4 and MDJ1; SSA4 and MDJ2; SSA4 and ERO1; SSA4 and ERV2; SSA4 and EUG1; SSA4 and MPD1; SSA4 and MPD2; SSA4 and PDI1; SSA4 and DER1; SSA4 and DER3; SSA4 and HRD3; SSA4 and UBC7; SSA4 and DOA4; SSA4 and HAC1; SSE1 and SSE2; SSE1 and SSB1; SSE1 and SSB2; SSE1 and ECM10; SSE1 and MDJ1; SSE1 and MDJ2; SSE1 and ERO1; SSE1 and ERV2; SSE1 and EUG1; SSE1 and MPD1; SSE1 and MPD2; SSE1 and PDI1; SSE1 and DER1; SSE1 and DER3; SSE1 and HRD3; SSE1 and UBC7; SSE1 and DOA4; SSE1 and HAC1; SSE2 and SSB1; SSE2 and SSB2; SSE2 and ECM10; SSE2 and MDJ1; SSE2 and MDJ2; SSE2 and ERO1; SSE2 and ERV2; SSE2 and EUG1; SSE2 and MPD1; SSE2 and MPD2; SSE2 and PDI1; SSE2 and DER1; SSE2 and DER3; SSE2 and HRD3; SSE2 and UBC7; SSE2 and DOA4; SSE2 and HAC1; SSB1 and SSB2; SSB1 and ECM10; SSB1 and MDJ1; SSB1 and MDJ2; SSB1 and ERO1; SSB1 and ERV2; SSB1 and EUG1; SSB1 and MPD1; SSB1 and MPD2; SSB1 and PDI1; SSB1 and DER1; SSB1 and DER3; SSB1 and HRD3; SSB1 and UBC7; SSB1 and DOA4; SSB1 and HAC1; SSB2 and ECM10; SSB2 and MDJ1; SSB2 and MDJ2; SSB2 and ERO1; SSB2 and ERV2; SSB2 and EUG1; SSB2 and MPD1; SSB2 and MPD2; SSB2 and PDI1; SSB2 and DER1; SSB2 and DER3; SSB2 and HRD3; SSB2 and UBC7; SSB2 and DOA4; SSB2 and HAC1; ECM10 and MDJ1; ECM10 and MDJ2; ECM10 and ERO1; ECM10 and ERV2; ECM10 and EUG1; ECM10 and MPD1; ECM10 and MPD2; ECM10 and PDI1; ECM10 and DER1; ECM10 and DER3; ECM10 and HRD3; ECM10 and UBC7; ECM10 and DOA4; ECM10 and HAM; MDJ1 and MDJ2; MDJ1 and ERO1; MDJ1 and ERV2; MDJ1 and EUG1; MDJ1 and MPD1; MDJ1 and MPD2; MDJ1 and PDI1; MDJ1 and DER1; MDJ1 and DER3; MDJ1 and HRD3; MDJ1 and UBC7; MDJ1 and DOA4; MDJ1 and HAC1; MDJ2 and ERO1; MDJ2 and ERV2; MDJ2 and EUG1; MDJ2 and MPD1; MDJ2 and MPD2; MDJ2 and PDI1; MDJ2 and DER1; MDJ2 and DER3; MDJ2 and HRD3; MDJ2 and UBC7; MDJ2 and DOA4; MDJ2 and HAC1; ERO1 and ERV2; ERO1 and EUG1; ERO1 and MPD1; ERO1 and MPD2; ERO1 and PDI1; ERO1 and DER1; ERO1 and DER3; ERO1 and HRD3; ERO1 and UBC7; ERO1 and DOA4; ERO1 and HAC1; ERV2 and EUG1; ERV2 and MPD1; ERV2 and MPD2; ERV2 and PDI1; ERV2 and DER1; ERV2 and DER3; ERV2 and HRD3; ERV2 and UBC7; ERV2 and DOA4; ERV2 and HAC1; EUG1 and MPD1; EUG1 and MPD2; EUG1 and PDI1; EUG1 and DER1; EUG1 and DER3; EUG1 and HRD3; EUG1 and UBC7; EUG1 and DOA4; EUG1 and HAC1; MPD1 and MPD2; MPD1 and PDI1; MPD1 and DER1; MPD1 and DER3; MPD1 and HRD3; MPD1 and UBC7; MPD1 and DOA4; MPD1 and HAC1; MPD2 and PDI1; MPD2 and DER1; MPD2 and DER3; MPD2 and HRD3; MPD2 and UBC7; MPD2 and DOA4; MPD2 and HAC1; PDI1 and DER1; PDI1 and DER3; PDI1 and HRD3; PDI1 and UBC7; PDI1 and DOA4; PDI1 and HAC1; DER1 and DER3; DER1 and HRD3; DER1 and UBC7; DER1 and DOA4; DER1 and HAC1; DER3 and HRD3; DER3 and UBC7; DER3 and DOA4; DER3 and HAC1; HRD3 and UBC7; HRD3 and DOA4; HRD3 and HAC1; UBC7 and DOA4; UBC7 and HAC1; or DOA4 and HAC1.

PDI1 in combination with any one of the following combinations: JEM1 and LHS1; JEM1 and SCJ1; JEM1 and KAR2; JEM1 and SIL1; JEM1 and FKB2; JEM1 and SSA1; JEM1 and SSA2; JEM1 and SSA3; JEM1 and SSA4; JEM1 and SSE1; JEM1 and SSE2; JEM1 and SSB1; JEM1 and SSB2; JEM1 and ECM10; JEM1 and MDJ1; JEM1 and MDJ2; JEM1 and ERO1; JEM1 and ERV2; JEM1 and EUG1; JEM1 and MPD1; JEM1 and MPD2; JEM1 and EPS1; JEM1 and DER1; JEM1 and DER3; JEM1 and HRD3; JEM1 and UBC7; JEM1 and DOA4; JEM1 and HAC1; LHS1 and SCJ1; LHS1 and KAR2; LHS1 and SIL1 LHS1 and FKB2; LHS1 and SSA1; LHS1 and SSA2; LHS1 and SSA3; LHS1 and SSA4; LHS1 and SSE1; LHS1 and SSE2; LHS1 and SSB1; LHS1 and SSB2; LHS1 and ECM10; LHS1 and MDJ1; LHS1 and MDJ2; LHS1 and ERO1; LHS1 and ERV2; LHS1 and EUG1; LHS1 and MPD1; LHS1 and MPD2; LHS1 and EPS1; LHS1 and DER1; LHS1 and DER3; LHS1 and HRD3; LHS1 and UBC7; LHS1 and DOA4; LHS1 and HAC1; SCJ1 and KAR2; SCJ1 and SIL1; SCJ1 and FKB2; SCJ1 and SSA1; SCJ1 and SSA2; SCJ1 and SSA3; SCJ1 and SSA4; SCJ1 and SSE1; SCJ1 and SSE2; SCJ1 and SSB1; SCJ1 and SSB2; SCJ1 and ECM10; SCJ1 and MDJ1; SCJ1 and MDJ2; SCJ1 and ERO1; SCJ1 and ERV2; SCJ1 and EUG1; SCJ1 and MPD1; SCJ1 and MPD2; SCJ1 and EPS1; SCJ1 and DER1; SCJ1 and DER3; SCJ1 and HRD3; SCJ1 and UBC7; SCJ1 and DOA4; SCJ1 and HAC1; KAR2 and SIL1; KAR2 and FKB2; KAR2 and SSA1; KAR2 and SSA2; KAR2 and SSA3; KAR2 and SSA4; KAR2 and SSE1; KAR2 and SSE2; KAR2 and SSB1; KAR2 and SSB2; KAR2 and ECM10; KAR2 and MDJ1; KAR2 and MDJ2; KAR2 and ERO1; KAR2 and ERV2; KAR2 and EUG1; KAR2 and MPD1; KAR2 and MPD2; KAR2 and EPS1; KAR2 and DER1; KAR2 and DER3; KAR2 and HRD3; KAR2 and UBC7; KAR2 and DOA4; KAR2 and HAC1; SIL1 and FKB2; SIL1 and SSA1; SIL1 and SSA2; SIL1 and SSA3; SIL1 and SSA4; SIL1 and SSE1; SIL1 and SSE2; SIL1 and SSB1; SIL1 and SSB2; SIL1 and ECM10; SIL1 and MDJ1; SIL1 and MDJ2; SIL1 and ERO1; SIL1 and ERV2; SIL1 and EUG1; SIL1 and MPD1; SIL1 and MPD2; SIL1 and EPS1; SIL1 and DER1; SIL1 and DER3; SIL1 and HRD3; SIL1 and UBC7; SIL1 and DOA4; SIL1 and HAC1; FKB2 and SSA1; FKB2 and SSA2; FKB2 and SSA3; FKB2 and SSA4; FKB2 and SSE1; FKB2 and SSE2; FKB2 and SSB1; FKB2 and SSB2; FKB2 and ECM10; FKB2 and MDJ1; FKB2 and MDJ2; FKB2 and ERO1; FKB2 and ERV2; FKB2 and EUG1; FKB2 and MPD1; FKB2 and MPD2; FKB2 and EPS1; FKB2 and DER1; FKB2 and DER3; FKB2 and HRD3; FKB2 and UBC7; FKB2 and DOA4; FKB2 and HAC1; SSA1 and SSA2; SSA1 and SSA3; SSA1 and SSA4; SSA1 and SSE1; SSA1 and SSE2; SSA1 and SSB1; SSA1 and SSB2; SSA1 and ECM10; SSA1 and MDJ1; SSA1 and MDJ2; SSA1 and ERO1; SSA1 and ERV2; SSA1 and EUG1; SSA1 and MPD1; SSA1 and MPD2; SSA1 and EPS1; SSA1 and DER1; SSA1 and DER3; SSA1 and HRD3; SSA1 and UBC7; SSA1 and DOA4; SSA1 and HAC1; SSA2 and SSA3; SSA2 and SSA4; SSA2 and SSE1; SSA2 and SSE2; SSA2 and SSB1; SSA2 and SSB2; SSA2 and ECM10; SSA2 and MDJ1; SSA2 and MDJ2; SSA2 and ERO1; SSA2 and ERV2; SSA2 and EUG1; SSA2 and MPD1; SSA2 and MPD2; SSA2 and EPS1; SSA2, and DER1; SSA2 and DER3; SSA2 and HRD3; SSA2 and UBC7; SSA2 and DOA4; SSA2 and HAC1; SSA3 and SSA4; SSA3 and SSE1; SSA3 and SSE2; SSA3 and SSB1; SSA3 and SSB2; SSA3 and ECM10; SSA3 and MDJ1; SSA3 and MDJ2; SSA3 and ERO1; SSA3 and ERV2; SSA3 and EUG1; SSA3 and MPD1; SSA3 and MPD2; SSA3 and EPS1; SSA3 and DER1; SSA3 and DER3; SSA3 and HRD3; SSA3 and UBC7; SSA3 and DOA4; SSA3 and HAC1; SSA4 and SSE1; SSA4 and SSE2; SSA4 and SSB1; SSA4 and SSB2; SSA4 and ECM10; SSA4 and MDJ1; SSA4 and MDJ2; SSA4 and ERO1; SSA4 and ERV2; SSA4 and EUG1; SSA4 and MPD1; SSA4 and MPD2; SSA4 and EPS1; SSA4 and DER1; SSA4 and DER3; SSA4 and HRD3; SSA4 and UBC7; SSA4 and DOA4; SSA4 and HAC1; SSE1 and SSE2; SSE1 and SSB1; SSE1 and SSB2; SSE1 and ECM10; SSE1 and MDJ1; SSE1 and MDJ2; SSE1 and ERO1; SSE1 and ERV2; SSE1 and EUG1; SSE1 and MPD1; SSE1 and MPD2; SSE1 and EPS1; SSE1 and DER1; SSE1 and DER3; SSE1 and HRD3; SSE1 and UBC7; SSE1 and DOA4; SSE1 and HAC1; SSE2 and SSB1; SSE2 and SSB2; SSE2 and ECM10; SSE2 and MDJ1; SSE2 and MDJ2; SSE2 and ERO1; SSE2 and ERV2; SSE2 and EUG1; SSE2 and MPD1; SSE2 and MPD2; SSE2 and EPS1; SSE2 and DER1; SSE2 and DER3; SSE2 and HRD3; SSE2 and UBC7; SSE2 and DOA4; SSE2 and HAC1; SSB1 and SSB2; SSB1 and ECM10; SSB1 and MDJ1; SSB1 and MDJ2; SSB1 and ERO1; SSB1 and ERV2; SSB1 and EUG1; SSB1 and MPD1; SSB1 and MPD2; SSB1 and EPS1; SSB1 and DER1; SSB1 and DER3; SSB1 and HRD3; SSB1 and UBC7; SSB1 and DOA4; SSB1 and HAC1; SSB2 and ECM10; SSB2 and MDJ1; SSB2 and MDJ2; SSB2 and ERO1; SSB2 and ERV2; SSB2 and EUG1; SSB2 and MPD1; SSB2 and MPD2; SSB2 and EPS1; SSB2 and DER1; SSB2 and DER3; SSB2 and HRD3; SSB2 and UBC7; SSB2 and DOA4; SSB2 and HAC1; ECM10 and MDJ1; ECM10 and MDJ2; ECM10 and ERO1; ECM10 and ERV2; ECM10 and EUG1; ECM10 and MPD1; ECM10 and MPD2; ECM10 and EPS1; ECM10 and DER1; ECM10 and DER3; ECM10 and HRD3; ECM10 and UBC7; ECM10 and DOA4; ECM10 and HAC1; MDJ1 and MDJ2; MDJ1 and ERO1; MDJ1 and ERV2; MDJ1 and EUG1; MDJ1 and MPD1; MDJ1 and MPD2; MDJ1 and EPS1; MDJ1 and DER1; MDJ1 and DER3; MDJ1 and HRD3; MDJ1 and UBC7; MDJ1 and DOA4; MDJ1 and HAC1; MDJ2 and ERO1; MDJ2 and ERV2; MDJ2 and EUG1; MDJ2 and MPD1; MDJ2 and MPD2; MDJ2 and EPS1; MDJ2 and DER1; MDJ2 and DER3; MDJ2 and HRD3; MDJ2 and UBC7; MDJ2 and DOA4; MDJ2 and HAC1; ERO1 and ERV2; ERO1 and EUG1; ERO1 and MPD1; ERO1 and MPD2; ERO1 and EPS1; ERO1 and DER1; ERO1 and DER3; ERO1 and HRD3; ERO1 and UBC7; ERO1 and DOA4; ERO1 and HAC1; ERV2 and EUG1; ERV2 and MPD1; ERV2 and MPD2; ERV2 and EPS1; ERV2 and DER1; ERV2 and DER3; ERV2 and HRD3; ERV2 and UBC7; ERV2 and DOA4; ERV2 and HAC1; EUG1 and MPD1; EUG1 and MPD2; EUG1 and EPS1; EUG1 and DER1; EUG1 and DER3; EUG1 and HRD3; EUG1 and UBC7; EUG1 and DOA4; EUG1 and HAC1; MPD1 and MPD2; MPD1 and EPS1; MPD1 and DER1; MPD1 and DER3; MPD1 and HRD3; MPD1 and UBC7; MPD1 and DOA4; MPD1 and HAC1; MPD2 and EPS1; MPD2 and DER1; MPD2 and DER3; MPD2 and HRD3; MPD2 and UBC7; MPD2 and DOA4; MPD2 and HAC1; EPS1 and DER1; EPS1 and DER3; EPS1 and HRD3; EPS1 and UBC7; EPS1 and DOA4; EPS1 and HAC1; DER1 and DER3; DER1 and HRD3; DER1 and UBC7; DER1 and DOA4; DER1 and HAC1; DER3 and HRD3; DER3 and UBC7; DER3 and DOA4; DER3 and HAC1; HRD3 and UBC7; HRD3 and DOA4; HRD3 and HAC1; UBC7 and DOA4; UBC7 and HAC1; or DOA4 and HAC1.

DER1 in combination with any one of the following combinations: JEM1 and LHS1; JEM1 and SCJ1; JEM1 and KAR2; JEM1 and SIL1; JEM1 and FKB2; JEM1 and SSA1; JEM1 and SSA2; JEM1 and SSA3; JEM1 and SSA4; JEM1 and SSE1; JEM1 and SSE2; JEM1 and SSB1; JEM1 and SSB2; JEM1 and ECM10; JEM1 and MDJ1; JEM1 and MDJ2; JEM1 and ERO1; JEM1 and ERV2; JEM1 and EUG1; JEM1 and MPD1; JEM1 and MPD2; JEM1 and EPS1; JEM1 and DER1; JEM1 and DER3; JEM1 and HRD3; JEM1 and UBC7; JEM1 and DOA4; JEM1 and HAC1; LHS1 and SCJ1; LHS1 and KAR2; LHS1 and SIL1 LHS1 and FKB2; LHS1 and SSA1; LHS1 and SSA2; LHS1 and SSA3; LHS1 and SSA4; LHS1 and SSE1; LHS1 and SSE2; LHS1 and SSB1; LHS1 and SSB2; LHS1 and ECM10; LHS1 and MDJ1; LHS1 and MDJ2; LHS1 and ERO1; LHS1 and ERV2; LHS1 and EUG1; LHS1 and MPD1; LHS1 and MPD2; LHS1 and EPS1; LHS1 and DER1; LHS1 and DER3; LHS1 and HRD3; LHS1 and UBC7; LHS1 and DOA4; LHS1 and HAC1; SCJ1 and KAR2; SCJ1 and SIL1; SCJ1 and FKB2; SCJ1 and SSA1; SCJ1 and SSA2; SCJ1 and SSA3; SCJ1 and SSA4; SCJ1 and SSE1; SCJ1 and SSE2; SCJ1 and SSB1; SCJ1 and SSB2; SCJ1 and ECM10; SCJ1 and MDJ1; SCJ1 and MDJ2; SCJ1 and ERO1; SCJ1 and ERV2; SCJ1 and EUG1; SCJ1 and MPD1; SCJ1 and MPD2; SCJ1 and EPS1; SCJ1 and DER1; SCJ1 and DER3; SCJ1 and HRD3; SCJ1 and UBC7; SCJ1 and DOA4; SCJ1 and HAC1; KAR2 and SIL1; KAR2 and FKB2; KAR2 and SSA1; KAR2 and SSA2; KAR2 and SSA3; KAR2 and SSA4; KAR2 and SSE1; KAR2 and SSE2; KAR2 and SSB1; KAR2 and SSB2; KAR2 and ECM10; KAR2 and MDJ1; KAR2 and MDJ2; KAR2 and ERO1; KAR2 and ERV2; KAR2 and EUG1; KAR2 and MPD1; KAR2 and MPD2; KAR2 and EPS1; KAR2 and DER1; KAR2 and DER3; KAR2 and HRD3; KAR2 and UBC7; KAR2 and DOA4; KAR2 and HAC1; SIL1 and FKB2; SIL1 and SSA1; SIL1 and SSA2; SIL1 and SSA3; SIL1 and SSA4; SIL1 and SSE1; SIL1 and SSE2; SIL1 and SSB1; SIL1 and SSB2; SIL1 and ECM10; SIL1 and MDJ1; SIL1 and MDJ2; SIL1 and ERO1; SIL1 and ERV2; SIL1 and EUG1; SIL1 and MPD1; SIL1 and MPD2; SIL1 and EPS1; SIL1 and DER1; SIL1 and DER3; SIL1 and HRD3; SIL1 and UBC7; SIL1 and DOA4; SIL1 and HAC1; FKB2 and SSA1; FKB2 and SSA2; FKB2 and SSA3; FKB2 and SSA4; FKB2 and SSE1; FKB2 and SSE2; FKB2 and SSB1; FKB2 and SSB2; FKB2 and ECM10; FKB2 and MDJ1; FKB2 and MDJ2; FKB2 and ERO1; FKB2 and ERV2; FKB2 and EUG1; FKB2 and MPD1; FKB2 and MPD2; FKB2 and EPS1; FKB2 and DER1; FKB2 and DER3; FKB2 and HRD3; FKB2 and UBC7; FKB2 and DOA4; FKB2 and HAC1; SSA1 and SSA2; SSA1 and SSA3; SSA1 and SSA4; SSA1 and SSE1; SSA1 and SSE2; SSA1 and SSB1; SSA1 and SSB2; SSA1 and ECM10; SSA1 and MDJ1; SSA1 and MDJ2; SSA1 and ERO1; SSA1 and ERV2; SSA1 and EUG1; SSA1 and MPD1; SSA1 and MPD2; SSA1 and EPS1; SSA1 and DER1; SSA1 and DER3; SSA1 and HRD3; SSA1 and UBC7; SSA1 and DOA4; SSA1 and HAC1; SSA2 and SSA3; SSA2 and SSA4; SSA2 and SSE1; SSA2 and SSE2; SSA2 and SSB1; SSA2 and SSB2; SSA2 and ECM10; SSA2 and MDJ1; SSA2 and MDJ2; SSA2 and ERO1; SSA2 and ERV2; SSA2 and EUG1; SSA2 and MPD1; SSA2 and MPD2; SSA2 and EPS1; SSA2 and DER1; SSA2 and DER3; SSA2 and HRD3; SSA2 and UBC7; SSA2 and DOA4; SSA2 and HAC1; SSA3 and SSA4; SSA3 and SSE1; SSA3 and SSE2; SSA3 and SSB1; SSA3 and SSB2; SSA3 and ECM10; SSA3 and MDJ1; SSA3 and MDJ2; SSA3 and ERO1; SSA3 and ERV2; SSA3 and EUG1; SSA3 and MPD1; SSA3 and MPD2; SSA3 and EPS1; SSA3 and DER1; SSA3 and DER3; SSA3 and HRD3; SSA3 and UBC7; SSA3 and DOA4; SSA3 and HAC1; SSA4 and SSE1; SSA4 and SSE2; SSA4 and SSB1; SSA4 and SSB2; SSA4 and ECM10; SSA4 and MDJ1; SSA4 and MDJ2; SSA4 and ERO1; SSA4 and ERV2; SSA4 and EUG1; SSA4 and MPD1; SSA4 and MPD2; SSA4 and EPS1; SSA4 and DER1; SSA4 and DER3; SSA4 and HRD3; SSA4 and UBC7; SSA4 and DOA4; SSA4 and HAC1; SSE1 and SSE2; SSE1 and SSB1; SSE1 and SSB2; SSE1 and ECM10; SSE1 and MDJ1; SSE1 and MDJ2; SSE1 and ERO1; SSE1 and ERV2; SSE1 and EUG1; SSE1 and MPD1; SSE1 and MPD2; SSE1 and EPS1; SSE1 and DER1; SSE1 and DER3; SSE1 and HRD3; SSE1 and UBC7; SSE1 and DOA4; SSE1 and HAC1; SSE2 and SSB1; SSE2 and SSB2; SSE2 and ECM10; SSE2 and MDJ1; SSE2 and MDJ2; SSE2 and ERO1; SSE2 and ERV2; SSE2 and EUG1; SSE2 and MPD1; SSE2 and MPD2; SSE2 and EPS1; SSE2 and DER1; SSE2 and DER3; SSE2 and HRD3; SSE2 and UBC7; SSE2 and DOA4; SSE2 and HAC1; SSB1 and SSB2; SSB1 and ECM10; SSB1 and MDJ1; SSB1 and MDJ2; SSB1 and ERO1; SSB1 and ERV2; SSB1 and EUG1; SSB1 and MPD1; SSB1 and MPD2; SSB1 and EPS1; SSB1 and DER1; SSB1 and DER3; SSB1 and HRD3; SSB1 and UBC7; SSB1 and DOA4; SSB1 and HAC1; SSB2 and ECM10; SSB2 and MDJ1; SSB2 and MDJ2; SSB2 and ERO1; SSB2 and ERV2; SSB2 and EUG1; SSB2 and MPD1; SSB2 and MPD2; SSB2 and EPS1; SSB2 and DER1; SSB2 and DER3; SSB2 and HRD3; SSB2 and UBC7; SSB2 and DOA4; SSB2 and HAC1; ECM10 and MDJ1; ECM10 and MDJ2; ECM10 and ERO1; ECM10 and ERV2; ECM10 and EUG1; ECM10 and MPD1; ECM10 and MPD2; ECM10 and EPS1; ECM10 and DER1; ECM10 and DER3; ECM10 and HRD3; ECM10 and UBC7; ECM10 and DOA4; ECM10 and HAC1; MDJ1 and MDJ2; MDJ1 and ERO1; MDJ1 and ERV2; MDJ1 and EUG1; MDJ1 and MPD1; MDJ1 and MPD2; MDJ1 and EPS1; MDJ1 and DER1; MDJ1 and DER3; MDJ1 and HRD3; MDJ1 and UBC7; MDJ1 and DOA4; MDJ1 and HAC1; MDJ2 and ERO1; MDJ2 and ERV2; MDJ2 and EUG1; MDJ2 and MPD1; MDJ2 and MPD2; MDJ2 and EPS1; MDJ2 and DER1; MDJ2 and DER3; MDJ2 and HRD3; MDJ2 and UBC7; MDJ2 and DOA4; MDJ2 and HAC1; ERO1 and ERV2; ERO1 and EUG1; ERO1 and MPD1; ERO1 and MPD2; ERO1 and EPS1; ERO1 and DER1; ERO1 and DER3; ERO1 and HRD3; ERO1 and UBC7; ERO1 and DOA4; ERO1 and HAC1; ERV2 and EUG1; ERV2 and MPD1; ERV2 and MPD2; ERV2 and EPS1; ERV2 and DER1; ERV2 and DER3; ERV2 and HRD3; ERV2 and UBC7; ERV2 and DOA4; ERV2 and HAC1; EUG1 and MPD1; EUG1 and MPD2; EUG1 and EPS1; EUG1 and DER1; EUG1 and DER3; EUG1 and HRD3; EUG1 and UBC7; EUG1 and DOA4; EUG1 and HAC1; MPD1 and MPD2; MPD1 and EPS1; MPD1 and DER1; MPD1 and DER3; MPD1 and HRD3; MPD1 and UBC7; MPD1 and DOA4; MPD1 and HAC1; MPD2 and EPS1; MPD2 and DER1; MPD2 and DER3; MPD2 and HRD3; MPD2 and UBC7; MPD2 and DOA4; MPD2 and HAC1; EPS1 and DER1; EPS1 and DER3; EPS1 and HRD3; EPS1 and UBC7; EPS1 and DOA4; EPS1 and HAC1; DER1 and DER3; DER1 and HRD3; DER1 and UBC7; DER1 and DOA4; DER1 and HAC1; DER3 and HRD3; DER3 and UBC7; DER3 and DOA4; DER3 and HAC1; HRD3 and UBC7; HRD3 and DOA4; HRD3 and HAC1; UBC7 and DOA4; UBC7 and HAC1; or DOA4 and HAC1.

DER3 in combination with any one of the following combinations: JEM1 and LHS1; JEM1 and SCJ1; JEM1 and KAR2; JEM1 and SIL1; JEM1 and FKB2; JEM1 and SSA1; JEM1 and SSA2; JEM1 and SSA3; JEM1 and SSA4; JEM1 and SSE1; JEM1 and SSE2; JEM1 and SSB1; JEM1 and SSB2; JEM1 and ECM10; JEM1 and MDJ1; JEM1 and MDJ2; JEM1 and ERO1; JEM1 and ERV2; JEM1 and EUG1; JEM1 and MPD1; JEM1 and MPD2; JEM1 and EPS1; JEM1 and PDI1; JEM1 and DER1; JEM1 and HRD3; JEM1 and UBC7; JEM1 and DOA4; JEM1 and HAC1; LHS1 and SCJ1; LHS1 and KAR2; LHS1 and SIL1; LHS1 and FKB2; LHS1 and SSA1; LHS1 and SSA2; LHS1 and SSA3; LHS1 and SSA4; LHS1 and SSE1; LHS1 and SSE2; LHS1 and SSB1; LHS1 and SSB2; LHS1 and ECM10; LHS1 and MDJ1; LHS1 and MDJ2; LHS1 and ERO1; LHS1 and ERV2; LHS1 and EUG1; LHS1 and MPD1; LHS1 and MPD2; LHS1 and EPS1; LHS1 and PDI1; LHS1 and DER1; LHS1 and HRD3; LHS1 and UBC7; LHS1 and DOA4; LHS1 and HAC1; SCJ1 and KAR2; SCJ1 and SIL1; SCJ1 and FKB2; SCJ1 and SSA1; SCJ1 and SSA2; SCJ1 and SSA3; SCJ1 and SSA4; SCJ1 and SSE1; SCJ1 and SSE2; SCJ1 and SSB1; SCJ1 and SSB2; SCJ1 and ECM10; SCJ1 and MDJ1; SCJ1 and MDJ2; SCJ1 and ERO1; SCJ1 and ERV2; SCJ1 and EUG1; SCJ1 and MPD1; SCJ1 and MPD2; SCJ1 and EPS1; SCJ1 and PDI1; SCJ1 and DER1; SCJ1 and HRD3; SCJ1 and UBC7; SCJ1 and DOA4; SCJ1 and HAC1; KAR2 and SIL1; KAR2 and FKB2; KAR2 and SSA1; KAR2 and SSA2; KAR2 and SSA3; KAR2 and SSA4; KAR2 and SSE1; KAR2 and SSE2; KAR2 and SSB1; KAR2 and SSB2; KAR2 and ECM10; KAR2 and MDJ1; KAR2 and MDJ2; KAR2 and ERO1; KAR2 and ERV2; KAR2 and EUG1; KAR2 and MPD1; KAR2 and MPD2; KAR2 and EPS1; KAR2 and PDI1; KAR2 and DER1; KAR2 and HRD3; KAR2 and UBC7; KAR2 and DOA4; KAR2 and HAC1; and FKB2; SIL1 and SSA1; SIL1 and SSA2; SIL1 and SSA3; SIL1 and SSA4; SIL1 and SSE1; SIL1 and SSE2; SIL1 and SSB1; SIL1 and SSB2; SIL1 and ECM10; SIL1 and MDJ1; SIL1 and MDJ2; SIL1 and ERO1; SIL1 and ERV2; SIL1 and EUG1; SIL1 and MPD1; SIL1 and MPD2; SIL1 and EPS1; SIL1 and PDI1; SIL1 and DER1; SIL1 and HRD3; SIL1 and UBC7; SIL1 and DOA4; SIL1 and HAC1; FKB2 and SSA1; FKB2 and SSA2; FKB2 and SSA3; FKB2 and SSA4; FKB2 and SSE1; FKB2 and SSE2; FKB2 and SSB1; FKB2 and SSB2; FKB2 and ECM10; FKB2 and MDJ1; FKB2 and MDJ2; FKB2 and ERO1; FKB2 and ERV2; FKB2 and EUG1; FKB2 and MPD1; FKB2 and MPD2; FKB2 and EPS1; FKB2 and PDI1; FKB2 and DER1; FKB2 and HRD3; FKB2 and UBC7; FKB2 and DOA4; FKB2 and HAC1; SSA1 and SSA2; SSA1 and SSA3; SSA1 and SSA4; SSA1 and SSE1; SSA1 and SSE2; SSA1 and SSB1; SSA1 and SSB2; SSA1 and ECM10; SSA1 and MDJ1; SSA1 and MDJ2; SSA1 and ERO1; SSA1 and ERV2; SSA1 and EUG1; SSA1 and MPD1; SSA1 and MPD2; SSA1 and EPS1; SSA1 and PDI1; SSA1 and DER1; SSA1 and HRD3; SSA1 and UBC7; SSA1 and DOA4; SSA1 and HAC1; SSA2 and SSA3; SSA2 and SSA4; SSA2 and SSE1; SSA2 and SSE2; SSA2 and SSB1; SSA2 and SSB2; SSA2 and ECM10; SSA2 and MDJ1; SSA2 and MDJ2; SSA2 and ERO1; SSA2 and ERV2; SSA2 and EUG1; SSA2 and MPD1; SSA2 and MPD2; SSA2 and EPS1; SSA2 and PDI1; SSA2 and DER1; SSA2 and HRD3; SSA2 and UBC7; SSA2 and DOA4; SSA2 and HAC1; SSA3 and SSA4; SSA3 and SSE1; SSA3 and SSE2; SSA3 and SSB1; SSA3 and SSB2; SSA3 and ECM10; SSA3 and MDJ1; SSA3 and MDJ2; SSA3 and ERO1; SSA3 and ERV2; SSA3 and EUG1; SSA3 and MPD1; SSA3 and MPD2; SSA3 and EPS1; SSA3 and PDI1; SSA3 and DER1; SSA3 and HRD3; SSA3 and UBC7; SSA3 and DOA4; SSA3 and HAC1; SSA4 and SSE1; SSA4 and SSE2; SSA4 and SSB1; SSA4 and SSB2; SSA4 and ECM10; SSA4 and MDJ1; SSA4 and MDJ2; SSA4 and ERO1; SSA4 and ERV2; SSA4 and EUG1; SSA4 and MPD1; SSA4 and MPD2; SSA4 and EPS1; SSA4 and PDI1; SSA4 and DER1; SSA4 and HRD3; SSA4 and UBC7; SSA4 and DOA4; SSA4 and HAC1; SSE1 and SSE2; SSE1 and SSB1; SSE1 and SSB2; SSE1 and ECM10; SSE1 and MDJ1; SSE1 and MDJ2; SSE1 and ERO1; SSE1 and ERV2; SSE1 and EUG1; SSE1 and MPD1; SSE1 and MPD2; SSE1 and EPS1; SSE1 and PDI1; SSE1 and DER1; SSE1 and HRD3; SSE1 and UBC7; SSE1 and DOA4; SSE1 and HAC1; SSE2 and SSB1; SSE2 and SSB2; SSE2 and ECM10; SSE2 and MDJ1; SSE2 and MDJ2; SSE2 and ERO1; SSE2 and ERV2; SSE2 and EUG1; SSE2 and MPD1; SSE2 and MPD2; SSE2 and EPS1; SSE2 and PDI1; SSE2 and DER1; SSE2 and HRD3; SSE2 and UBC7; SSE2 and DOA4; SSE2 and HAC1; SSB1 and SSB2; SSB1 and ECM10; SSB1 and MDJ1; SSB1 and MDJ2; SSB1 and ERO1; SSB1 and ERV2; SSB1 and EUG1; SSB1 and MPD1; SSB1 and MPD2; SSB1 and EPS1; SSB1 and PDI1; SSB1 and DER1; SSB1 and HRD3; SSB1 and UBC7; SSB1 and DOA4; SSB1 and HAC1, SSB2 and ECM10; SSB2 and MDJ1; SSB2 and MDJ2; SSB2 and ERO1; SSB2 and ERV2; SSB2 and EUG1; SSB2 and MPD1; SSB2 and MPD2; SSB2 and EPS1; SSB2 and PDI1; SSB2 and DER1; SSB2 and HRD3; SSB2 and UBC7; SSB2 and DOA4; SSB2 and HAC1; ECM10 and MDJ1; ECM10 and MDJ2; ECM10 and ERO1; ECM10 and ERV2; ECM10 and EUG1; ECM10 and MPD1; ECM10 and MPD2; ECM10 and EPS1; ECM10 and PDI1; ECM10 and DER1; ECM10 and HRD3; ECM10 and UBC7; ECM10 and DOA4; ECM10 and HAC1; MDJ1 and MDJ2; MDJ1 and ERO1; MDJ1 and ERV2; MDJ1 and EUG1; MDJ1 and MPD1; MDJ1 and MPD2; MDJ1 and EPS1; MDJ1 and PDI1; MDJ1 and DER1; MDJ1 and HRD3; MDJ1 and UBC7; MDJ1 and DOA4; MDJ1 and HAC1; MDJ2 and ERO1; MDJ2 and ERV2; MDJ2 and EUG1; MDJ2 and MPD1; MDJ2 and MPD2; MDJ2 and EPS1; MDJ2 and PDI1; MDJ2 and DER1; MDJ2 and HRD3; MDJ2 and UBC7; MDJ2 and DOA4; MDJ2 and HAC1; ERO1 and ERV2; ERO1 and EUG1; ERO1 and MPD1; ERO1 and MPD2; ERO1 and EPS1; ERO1 and PDI1; ERO1 and DER1; ERO1 and HRD3; ERO1 and UBC7; ERO1 and DOA4; ERO1 and HAC1; ERV2 and EUG1; ERV2 and MPD1; ERV2 and MPD2; ERV2 and EPS1; ERV2 and PDI1; ERV2 and DER1; ERV2 and HRD3; ERV2 and UBC7; ERV2 and DOA4; ERV2 and HAC1; EUG1 and MPD1; EUG1 and MPD2; EUG1 and EPS1; EUG1 and PDI1; EUG1 and DER1; EUG1 and HRD3; EUG1 and UBC7; EUG1 and DOA4; EUG1 and HAC1; MPD1 and MPD2; MPD1 and EPS1; MPD1 and PDI1; MPD1 and DER1; MPD1 and HRD3; MPD1 and UBC7; MPD1 and DOA4; MPD1 and HAC1; MPD2 and EPS1; MPD2 and PDI1; MPD2 and DER1; MPD2 and HRD3; MPD2 and UBC7; MPD2 and DOA4; MPD2 and HAC1; EPS1 and PDI1; EPS1 and DER1; EPS1 and HRD3; EPS1 and UBC7; EPS1 and DOA4; EPS1 and HAC1; PDI1 and DER1; PDI1 and HRD3; PDI1 and UBC7; PDI1 and DOA4; PDI1 and HAC1; DER1 and HRD3; DER1 and UBC7; DER1 and DOA4; DER1 and HAC1; HRD3 and UBC7; HRD3 and DOA4; HRD3 and HAC1; UBC7 and DOA4; UBC7 and HAC1; or DOA4 and HAC1.

HRD3 in combination with any one of the following combinations: JEM1 and LHS1; JEM1 and SCJ1; JEM1 and KAR2; JEM1 and SIL1; JEM1 and FKB2; JEM1 and SSA1; JEM1 and SSA2; JEM1 and SSA3; JEM1 and SSA4; JEM1 and SSE1; JEM1 and SSE2; JEM1 and SSB1; JEM1 and SSB2; JEM1 and ECM10; JEM1 and MDJ1; JEM1 and MDJ2; JEM1 and ERO1; JEM1 and ERV2; JEM1 and EUG1; JEM1 and MPD1; JEM1 and MPD2; JEM1 and EPS1; JEM1 and PDI1; JEM1 and DER1; JEM1 and DER3; JEM1 and UBC7; JEM1 and DOA4; JEM1 and HAC1; LHS1 and SCJ1; LHS1 and KAR2; LHS1 and SIL1 LHS1 and FKB2; LHS1 and SSA1; LHS1 and SSA2; LHS1 and SSA3; LHS1 and SSA4; LHS1 and SSE1; LHS1 and SSE2; LHS1 and SSB1; LHS1 and SSB2; LHS1 and ECM10; LHS1 and MDJ1; LHS1 and MDJ2; LHS1 and ERO1; LHS1 and ERV2; LHS1 and EUG1; LHS1 and MPD1; LHS1 and MPD2; LHS1 and EPS1; LHS1 and PDI1; LHS1 and DER1; LHS1 and DER3; LHS1 and UBC7; LHS1 and DOA4; LHS1 and HAC1; SCJ1 and KAR2; SCJ1 and SIL1; SCJ1 and FKB2; SCJ1 and SSA1; SCJ1 and SSA2; SCJ1 and SSA3; SCJ1 and SSA4; SCJ1 and SSE1; SCJ1 and SSE2; SCJ1 and SSB1; SCJ1 and SSB2; SCJ1 and ECM10; SCJ1 and MDJ1; SCJ1 and MDJ2; SCJ1 and ERO1; SCJ1 and ERV2; SCJ1 and EUG1; SCJ1 and MPD1; SCJ1 and MPD2; SCJ1 and EPS1; SCJ1 and PDI1; SCJ1 and DER1; SCJ1 and DER3; SCJ1 and UBC7; SCJ1 and DOA4; SCJ1 and HAC1; KAR2 and SIL1; KAR2 and FKB2; KAR2 and SSA1; KAR2 and SSA2; KAR2 and SSA3; KAR2 and SSA4; KAR2 and SSE1; KAR2 and SSE2; KAR2 and SSB1; KAR2 and SSB2; KAR2 and ECM10; KAR2 and MDJ1; KAR2 and MDJ2; KAR2 and ERO1; KAR2 and ERV2; KAR2 and EUG1; KAR2 and MPD1; KAR2 and MPD2; KAR2 and EPS1; KAR2 and PDI1; KAR2 and DER1; KAR2 and DER3; KAR2 and UBC7; KAR2 and DOA4; KAR2 and HAC1; SIL1 and FKB2; SIL1 and SSA1; SIL1 and SSA2; SIL1 and SSA3; SIL1 and SSA4; SIL1 and SSE1; SIL1 and SSE2; SEM and SSB1; SIL1 and SSB2; SIL1 and ECM10; SIL1 and MDJ1; SIL1 and MDJ2; SIL1 and ERO1; SIL1 and ERV2; SIL1 and EUG1; SIL1 and MPD1; SIL1 and MPD2; SIL1 and EPS1; SIL1 and PDI1; SIL1 and DER1; SIL1 and DER3; SIL1 and UBC7; SIL1 and DOA4; SIL1 and HAC1; FKB2 and SSA1; FKB2 and SSA2; FKB2 and SSA3; FKB2 and SSA4; FKB2 and SSE1; FKB2 and SSE2; FKB2 and SSB1; FKB2 and SSB2; FKB2 and ECM10; FKB2 and MDJ1; FKB2 and MDJ2; FKB2 and ERO1; FKB2 and ERV2; FKB2 and EUG1; FKB2 and MPD1; FKB2 and MPD2; FKB2 and EPS1; FKB2 and PDI1; FKB2 and DER1; FKB2 and DER3; FKB2 and UBC7; FKB2 and DOA4; FKB2 and HAC1; SSA1 and SSA2; SSA1 and SSA3; SSA1 and SSA4; SSA1 and SSE1; SSA1 and SSE2; SSA1 and SSB1; SSA1 and SSB2; to SSA1 and ECM10; SSA1 and MDJ1; SSA1 and MDJ2; SSA1 and ERO1; SSA1 and ERV2; SSA1 and EUG1; SSA1 and MPD1; SSA1 and MPD2; SSA1 and EPS1; SSA1 and PDI1; SSA1 and DER1; SSA1 and DER3; SSA1 and UBC7; SSA1 and DOA4; SSA1 and HAC1; SSA2 and SSA3; SSA2 and SSA4; SSA2 and SSE1; SSA2 and SSE2; SSA2 and SSB1; SSA2 and SSB2; SSA2 and ECM10; SSA2 and MDJ1; SSA2 and MDJ2; SSA2 and ERO1; SSA2 and ERV2; SSA2 and EUG1; SSA2 and MPD1; SSA2 and MPD2; SSA2 and EPS1; SSA2 and PDI1; SSA2 and DER1; SSA2 and DER3; SSA2 and UBC7; SSA2 and DOA4; SSA2 and HAC1; SSA3 and SSA4; SSA3 and SSE1; SSA3 and SSE2; SSA3 and SSB1; SSA3 and SSB2; SSA3 and ECM10; SSA3 and MDJ1; SSA3 and MDJ2; SSA3 and ERO1; SSA3 and ERV2; SSA3 and EUG1; SSA3 and MPD1; SSA3 and MPD2; SSA3 and EPS1; SSA3 and PDI1; SSA3 and DER1; SSA3 and DER3; SSA3 and UBC7; SSA3 and DOA4; SSA3 and HAC1; SSA4 and SSE1; SSA4 and SSE2; SSA4 and SSB1; SSA4 and SSB2; SSA4 and ECM10; SSA4 and MDJ1; SSA4 and MDJ2; SSA4 and ERO1; SSA4 and ERV2; SSA4 and EUG1; SSA4 and MPD1; SSA4 and MPD2; SSA4 and EPS1; SSA4 and PDI1; SSA4 and DER1; SSA4 and DER3; SSA4 and UBC7; SSA4 and DOA4; SSA4 and HAC1; SSE1 and SSE2; SSE1 and SSB1; SSE1 and SSB2; SSE1 and ECM10; SSE1 and MDJ1; SSE1 and MDJ2; SSE1 and ERO1; SSE1 and ERV2; SSE1 and EUG1; SSE1 and MPD1; SSE1 and MPD2; SSE1 and EPS1; SSE1 and PDI1; SSE1 and DER1; SSE1 and DER3; SSE1 and UBC7; SSE1 and DOA4; SSE1 and HAC1; SSE2 and SSB1; SSE2 and SSB2; SSE2 and ECM10; SSE2 and MDJ1; SSE2 and MDJ2; SSE2 and ERO1; SSE2 and ERV2; SSE2 and EUG1; SSE2 and MPD1; SSE2 and MPD2; SSE2 and EPS1; SSE2 and PDI1; SSE2 and DER1; SSE2 and DER3; SSE2 and UBC7; SSE2 and DOA4; SSE2 and HAC1; SSB1 and SSB2; SSB1 and ECM10; SSB1 and MDJ1; SSB1 and MDJ2; SSB1 and ERO1; SSB1 and ERV2; SSB1 and EUG1; SSB1 and MPD1; SSB1 and MPD2; SSB1 and EPS1; SSB1 and PDI1; SSB1 and DER1; SSB1 and DER3; SSB1 and UBC7; SSB1 and DOA4; SSB1 and HAC1; SSB2 and ECM10; SSB2 and MDJ1; SSB2 and MDJ2; SSB2 and ERO1; SSB2 and ERV2; SSB2 and EUG1; SSB2 and MPD1; SSB2 and MPD2; SSB2 and EPS1; SSB2 and PDI1; SSB2 and DER1; SSB2 and DER3; SSB2 and UBC7; SSB2 and DOA4; SSB2 and HAC1; ECM10 and MDJ1; ECM10 and MDJ2; ECM10 and ERO1; ECM10 and ERV2; ECM10 and EUG1; ECM10 and MPD1; ECM10 and MPD2; ECM10 and EPS1; ECM10 and PDI1; ECM10 and DER1; ECM10 and DER3; ECM10 and UBC7; ECM10 and DOA4; ECM10 and HAC1; MDJ1 and MDJ2; MDJ1 and ERO1; MDJ1 and ERV2; MDJ1 and EUG1; MDJ1 and MPD1; MDJ1 and MPD2; MDJ1 and EPS1; MDJ1 and PDI1; MDJ1 and DER1; MDJ1 and DER3; MDJ1 and UBC7; MDJ1 and DOA4; MDJ1 and HAC1; MDJ2 and ERO1; MDJ2 and ERV2; MDJ2 and EUG1; MDJ2 and MPD1; MDJ2 and MPD2; MDJ2 and EPS1; MDJ2 and PDI1; MDJ2 and DER1; MDJ2 and DER3; MDJ2 and UBC7; MDJ2 and DOA4; MDJ2 and HAC1; ERO1 and ERV2; ERO1 and EUG1; ERO1 and MPD1; ERO1 and MPD2; ERO1 and EPS1; ERO1 and PDI1; ERO1 and DER1; ERO1 and DER3; ERO1 and UBC7; ERO1 and DOA4; ERO1 and HAC1; ERV2 and EUG1; ERV2 and MPD1; ERV2 and MPD2; ERV2 and EPS1; ERV2 and PDI1; ERV2 and DER1; ERV2 and DER3; ERV2 and UBC7; ERV2 and DOA4; ERV2 and HAC1; EUG1 and MPD1; EUG1 and MPD2; EUG1 and EPS1; EUG1 and PDI1; EUG1 and DER1; EUG1 and DER3; EUG1 and UBC7; EUG1 and DOA4; EUG1 and HAC1; MPD1 and MPD2; MPD1 and EPS1; MPD1 and PDI1; MPD1 and DER1; MPD1 and DER3; MPD1 and UBC7; MPD1 and DOA4; MPD1 and HAC1; MPD2 and EPS1; MPD2 and PDI1; MPD2 and DER1; MPD2 and DER3; MPD2 and UBC7; MPD2 and DOA4; MPD2 and HAC1; EPS1 and PDI1; EPS1 and DER1; EPS1 and DER3; EPS1 and UBC7; EPS1 and DOA4; EPS1 and HAC1; PDI1 and DER1; PDI1 and DER3; PDI1 and UBC7; PDI1 and DOA4; PDI1 and HAC1; DER1 and DER3; DER1 and UBC7; DER1 and DOA4; DER1 and HAC1; DER3 and UBC7; DER3 and DOA4; DER3 and HAC1; UBC7 and DOA4; UBC7 and HAC1; or DOA4 and HAC1.

UBC7 in combination with any one of the following combinations: JEM1 and LHS1; JEM1 and SCJ1; JEM1 and KAR2; JEM1 and SIL1; JEM1 and FKB2; JEM1 and SSA1; JEM1 and SSA2; JEM1 and SSA3; JEM1 and SSA4; JEM1 and SSE1; JEM1 and SSE2; JEM1 and SSB1; JEM1 and SSB2; JEM1 and ECM10; JEM1 and MDJ1; JEM1 and MDJ2; JEM1 and ERO1; JEM1 and ERV2; JEM1 and EUG1; JEM1 and MPD1; JEM1 and MPD2; JEM1 and EPS1; JEM1 and PDI1; JEM1 and DER1; JEM1 and DER3; JEM1 and HRD3; JEM1 and DOA4; JEM1 and HAC1; LHS1 and SCJ1; LHS1 and KAR2; LHS1 and SIL1 LHS1 and FKB2; LHS1 and SSA1; LHS1 and SSA2; LHS1 and SSA3; LHS1 and SSA4; LHS1 and SSE1; LHS1 and SSE2; LHS1 and SSB1; LHS1 and SSB2; LHS1 and ECM10; LHS1 and MDJ1; LHS1 and MDJ2; LHS1 and ERO1; LHS1 and ERV2; LHS1 and EUG1; LHS1 and MPD1; LHS1 and MPD2; LHS1 and EPS1; LHS1 and PDI1; LHS1 and DER1; LHS1 and DER3; LHS1 and HRD3; LHS1 and DOA4; LHS1 and HAC1; SCJ1 and KAR2; SCJ1 and SIL1; SCJ1 and FKB2; SCJ1 and SSA1; SCJ1 and SSA2; SCJ1 and SSA3; SCJ1 and SSA4; SCJ1 and SSE1; SCJ1 and SSE2; SCJ1 and SSB1; SCJ1 and SSB2; SCJ1 and ECM10; SCJ1 and MDJ1; SCJ1 and MDJ2; SCJ1 and ERO1; SCJ1 and ERV2; SCJ1 and EUG1; SCJ1 and MPD1; SCJ1 and MPD2; SCJ1 and EPS1; SCJ1 and PDI1; SCJ1 and DER1; SCJ1 and DER3; SCJ1 and HRD3; SCJ1 and DOA4; SCJ1 and HAC1; KAR2 and SIL1; KAR2 and FKB2; KAR2 and SSA1; KAR2 and SSA2; KAR2 and SSA3; KAR2 and SSA4; KAR2 and SSE1; KAR2 and SSE2; KAR2 and SSB1; KAR2 and SSB2; KAR2 and ECM10; KAR2 and MDJ1; KAR2 and MDJ2; KAR2 and ERO1; KAR2 and ERV2; KAR2 and EUG1; KAR2 and MPD1; KAR2 and MPD2; KAR2 and EPS1; KAR2 and PDI1; KAR2 and DER1; KAR2 and DER3; KAR2 and HRD3; KAR2 and DOA4; KAR2 and HAC1; SIL1 and FKB2; SIL1 and SSA1; SIL1 and SSA2; SIL1 and SSA3; SIL1 and SSA4; SIL1 and SSE1; SIL1 and SSE2; SIL1 and SSB1; SIL1 and SSB2; SIL1 and ECM10; SIL1 and MDJ1; SIL1 and MDJ2; SIL1 and ERO1; SIL1 and ERV2; SIL1 and EUG1; SIL1 and MPD1; SIL1 and MPD2; SIL1 and EPS1; SIL1 and PDI1; SIL1 and DER1; SIL1 and DER3; SIL1 and HRD3; SIL1 and DOA4; SIL1 and HAC1; FKB2 and SSA1; FKB2 and SSA2; FKB2 and SSA3; FKB2 and SSA4; FKB2 and SSE1; FKB2 and SSE2; FKB2 and SSB1; FKB2 and SSB2; FKB2 and ECM10; FKB2 and MDJ1; FKB2 and MDJ2; FKB2 and ERO1; FKB2 and ERV2; FKB2 and EUG1; FKB2 and MPD1; FKB2 and MPD2; FKB2 and EPS1; FKB2 and PDI1; FKB2 and DER1; FKB2 and DER3; FKB2 and HRD3; FKB2 and DOA4; FKB2 and HAC1; SSA1 and SSA2; SSA1 and SSA3; SSA1 and SSA4; SSA1 and SSE1; SSA1 and SSE2; SSA1 and SSB1; SSA1 and SSB2; SSA1 and ECM10; SSA1 and MDJ1; SSA1 and MDJ2; SSA1 and ERO1; SSA1 and ERV2; SSA1 and EUG1; SSA1 and MPD1; SSA1 and MPD2; SSA1 and EPS1; SSA1 and PDI1; SSA1 and DER1; SSA1 and DER3; SSA1 and HRD3; SSA1 and DOA4; SSA1 and HAC1; SSA2 and SSA3; SSA2 and SSA4; SSA2 and SSE1; SSA2 and SSE2; SSA2 and SSB1; SSA2 and SSB2; SSA2 and ECM10; SSA2 and MDJ1; SSA2 and MDJ2; SSA2 and ERO1; SSA2 and ERV2; SSA2 and EUG1; SSA2 and MPD1; SSA2 and MPD2; SSA2 and EPS1; SSA2 and PDI1; SSA2 and DER1; SSA2 and DER3; SSA2 and HRD3; SSA2 and DOA4; SSA2 and HAC1; SSA3 and SSA4; SSA3 and SSE1; SSA3 and SSE2; SSA3 and SSB1; SSA3 and SSB2; SSA3 and ECM10; SSA3 and MDJ1; SSA3 and MDJ2; SSA3 and ERO1; SSA3 and ERV2; SSA3 and EUG1; SSA3 and MPD1; SSA3 and MPD2; SSA3 and EPS1; SSA3 and PDI1; SSA3 and DER1; SSA3 and DER3; SSA3 and HRD3; SSA3 and DOA4; SSA3 and HAC1; SSA4 and SSE1; SSA4 and SSE2; SSA4 and SSB1; SSA4 and SSB2; SSA4 and ECM10; SSA4 and MDJ1; SSA4 and MDJ2; SSA4 and ERO1; SSA4 and ERV2; SSA4 and EUG1; SSA4 and MPD1; SSA4 and MPD2; SSA4 and EPS1; SSA4 and PDI1; SSA4 and DER1; SSA4 and DER3; SSA4 and HRD3; SSA4 and DOA4; SSA4 and HAC1; SSE1 and SSE2; SSE1 and SSB1; SSE1 and SSB2; SSE1 and ECM10; SSE1 and MDJ1; SSE1 and MDJ2; SSE1 and ERO1; SSE1 and ERV2; SSE1 and EUG1; SSE1 and MPD1; SSE1 and MPD2; SSE1 and EPS1; SSE1 and PDI1; SSE1 and DER1; SSE1 and DER3; SSE1 and HRD3; SSE1 and DOA4; SSE1 and HAC1; SSE2 and SSB1; SSE2 and SSB2; SSE2 and ECM10; SSE2 and MDJ1; SSE2 and MDJ2; SSE2 and ERO1; SSE2 and ERV2; SSE2 and EUG1; SSE2 and MPD1; SSE2 and MPD2; SSE2 and EPS1; SSE2 and PDI1; SSE2 and DER1; SSE2 and DER3; SSE2 and HRD3; SSE2 and DOA4; SSE2 and HAC1; SSB1 and SSB2; SSB1 and ECM10; SSB1 and MDJ1; SSB1 and MDJ2; SSB1 and ERO1; SSB1 and ERV2; SSB1 and EUG1; SSB1 and MPD1; SSB1 and MPD2; SSB1 and EPS1; SSB1 and PDI1; SSB1 and DER1; SSB1 and DER3; SSB1 and HRD3; SSB1 and DOA4; SSB1 and HAC1; SSB2 and ECM10; SSB2 and MDJ1; SSB2 and MDJ2; SSB2 and ERO1; SSB2 and ERV2; SSB2 and EUG1; SSB2 and MPD1; SSB2 and MPD2; SSB2 and EPS1; SSB2 and PDI1; SSB2 and DER1; SSB2 and DER3; SSB2 and HRD3; SSB2 and DOA4; SSB2 and HAC1; ECM10 and MDJ1; ECM10 and MDJ2; ECM10 and ERO1; ECM10 and ERV2; ECM10 and EUG1; ECM10 and MPD1; ECM10 and MPD2; ECM10 and EPS1; ECM10 and PDI1; ECM10 and DER1; ECM10 and DER3; ECM10 and HRD3; ECM10 and DOA4; ECM10 and HAC1; MDJ1 and MDJ2; MDJ1 and ERO1; MDJ1 and ERV2; MDJ1 and EUG1; MDJ1 and MPD1; MDJ1 and MPD2; MDJ1 and EPS1; MDJ1 and PDI1; MDJ1 and DER1; MDJ1 and DER3; MDJ1 and HRD3; MDJ1 and DOA4; MDJ1 and HAC1; MDJ2 and ERO1; MDJ2 and ERV2; MDJ2 and EUG1; MDJ2 and MPD1; MDJ2 and MPD2; MDJ2 and EPS1; MDJ2 and PDI1; MDJ2 and DER1; MDJ2 and DER3; MDJ2 and HRD3; MDJ2 and DOA4; MDJ2 and HAC1; ERO1 and ERV2; ERO1 and EUG1; ERO1 and MPD1; ERO1 and MPD2; ERO1 and EPS1; ERO1 and PDI1; ERO1 and DER1; ERO1 and DER3; ERO1 and HRD3; ERO1 and DOA4; ERO1 and HAC1; ERV2 and EUG1; ERV2 and MPD1; ERV2 and MPD2; ERV2 and EPS1; ERV2 and PDI1; ERV2 and DER1; ERV2 and DER3; ERV2 and HRD3; ERV2 and DOA4; ERV2 and HAC1; EUG1 and MPD1; EUG1 and MPD2; EUG1 and EPS1; EUG1 and PDI1; EUG1 and DER1; EUG1 and DER3; EUG1 and HRD3; EUG1 and DOA4; EUG1 and HAC1; MPD1 and MPD2; MPD1 and EPS1; MPD1 and PDI1; MPD1 and DER1; MPD1 and DER3; MPD1 and HRD3; MPD1 and DOA4; MPD1 and HAC1; MPD2 and EPS1; MPD2 and PDI1; MPD2 and DER1; MPD2 and DER3; MPD2 and HRD3; MPD2 and DOA4; MPD2 and HAC1; EPS1 and PDI1; EPS1 and DER1; EPS1 and DER3; EPS1 and HRD3; EPS1 and DOA4; EPS1 and HAC1; PDI1 and DER1; PDI1 and DER3; PDI1 and HRD3; PDI1 and DOA4; PDI1 and HAC1; DER1 and DER3; DER1 and HRD3; DER1 and DOA4; DER1 and HAC1; DER3 and HRD3; DER3 and DOA4; DER3 and HAC1; HRD3 and DOA4; HRD3 and HAC1; or DOA4 and HAC1.

DOA4 in combination with any one of the following combinations: JEM1 and LHS1; JEM1 and SCJ1; JEM1 and KAR2; JEM1 and SIL1; JEM1 and FKB2; JEM1 and SSA1; JEM1 and SSA2; JEM1 and SSA3; JEM1 and SSA4; JEM1 and SSE1; JEM1 and SSE2; JEM1 and SSB1; JEM1 and SSB2; JEM1 and ECM10; JEM1 and MDJ1; JEM1 and MDJ2; JEM1 and ERO1; JEM1 and ERV2; JEM1 and EUG1; JEM1 and MPD1; JEM1 and MPD2; JEM1 and EPS1; JEM1 and PDI1; JEM1 and DER1; JEM1 and DER3; JEM1 and HRD3; JEM1 and UBC7; JEM1 and HAC1; LHS1 and SCJ1; LHS1 and KAR2; LHS1 and SIL1; LHS1 and FKB2; LHS1 and SSA1; LHS1 and SSA2; LHS1 and SSA3; LHS1 and SSA4; LHS1 and SSE1; LHS1 and SSE2; LHS1 and SSB1; LHS1 and SSB2; LHS1 and ECM10; LHS1 and MDJ1; LHS1 and MDJ2; LHS1 and ERO1; LHS1 and ERV2; LHS1 and EUG1; LHS1 and MPD1; LHS1 and MPD2; LHS1 and EPS1; LHS1 and PDI1; LHS1 and DER1; LHS1 and DER3; LHS1 and HRD3; LHS1 and UBC7; LHS1 and HAC1; SCJ1 and KAR2; SCJ1 and SIL1; SCJ1 and FKB2; SCJ1 and SSA1; SCJ1 and SSA2; SCJ1 and SSA3; SCJ1 and SSA4; SCJ1 and SSE1; SCJ1 and SSE2; SCJ1 and SSB1; SCJ1 and SSB2; SCJ1 and ECM10; SCJ1 and MDJ1; SCJ1 and MDJ2; SCJ1 and ERO1; SCJ1 and ERV2; SCJ1 and EUG1; SCJ1 and MPD1; SCJ1 and MPD2; SCJ1 and EPS1; SCJ1 and PDI1; SCJ1 and DER1; SCJ1 and DER3; SCJ1 and HRD3; SCJ1 and UBC7; SCJ1 and HAC1; KAR2 and SIL1; KAR2 and FKB2; KAR2 and SSA1; KAR2 and SSA2; KAR2 and SSA3; KAR2 and SSA4; KAR2 and SSE1; KAR2 and SSE2; KAR2 and SSB1; KAR2 and SSB2; KAR2 and ECM10; KAR2 and MDJ1; KAR2 and MDJ2; KAR2 and ERO1; KAR2 and ERV2; KAR2 and EUG1; KAR2 and MPD1; KAR2 and MPD2; KAR2 and EPS1; KAR2 and PDI1; KAR2 and DER1; KAR2 and DER3; KAR2 and HRD3; KAR2 and UBC7; KAR2 and HAC1; SIL1 and FKB2; SIL1 and SSA1; SIL1 and SSA2; SIL1 and SSA3; SIL1 and SSA4; SIL1 and SSE1; SIL1 and SSE2; SIL1 and SSB1; SIL1 and SSB2; SIL1 and ECM10; SIL1 and MDJ1; SIL1 and MDJ2; SIL1 and ERO1; SIL1 and ERV2; SIL1 and EUG1; SIL1 and MPD1; SIL1 and MPD2; SIL1 and EPS1; SIL1 and PDI1; SIL1 and DER1; SIL1 and DER3; SIL1 and HRD3; SIL1 and UBC7; SIL1 and HAC1; FKB2 and SSA1; FKB2 and SSA2; FKB2 and SSA3; FKB2 and SSA4; FKB2 and SSE1; FKB2 and SSE2; FKB2 and SSB1; FKB2 and SSB2; FKB2 and ECM10; FKB2 and MDJ1; FKB2 and MDJ2; FKB2 and ERO1; FKB2 and ERV2; FKB2 and EUG1; FKB2 and MPD1; FKB2 and MPD2; FKB2 and EPS1; FKB2 and PDI1; FKB2 and DER1; FKB2 and DER3; FKB2 and HRD3; FKB2 and UBC7; FKB2 and HAC1; SSA1 and SSA2; SSA1 and SSA3; SSA1 and SSA4; SSA1 and SSE1; SSA1 and SSE2; SSA1 and SSB1; SSA1 and SSB2; SSA1 and ECM10; SSA1 and MDJ1; SSA1 and MDJ2; SSA1 and ERO1; SSA1 and ERV2; SSA1 and EUG1; SSA1 and MPD1; SSA1 and MPD2; SSA1 and EPS1; SSA1 and PDI1; SSA1 and DER1; SSA1 and DER3; SSA1 and HRD3; SSA1 and UBC7; SSA1 and HAC1; SSA2 and SSA3; SSA2 and SSA4; SSA2 and SSE1; SSA2 and SSE2; SSA2 and SSB1; SSA2 and SSB2; SSA2 and ECM10; SSA2 and MDJ1; SSA2 and MDJ2; SSA2 and ERO1; SSA2 and ERV2; SSA2 and EUG1; SSA2 and MPD1; SSA2 and MPD2; SSA2 and EPS1; SSA2 and PDI1; SSA2 and DER1; SSA2 and DER3; SSA2 and HRD3; SSA2 and UBC7; SSA2 and HAC1; SSA3 and SSA4; SSA3 and SSE1; SSA3 and SSE2; SSA3 and SSB1; SSA3 and SSB2; SSA3 and ECM10; SSA3 and MDJ1; SSA3 and MDJ2; SSA3 and ERO1; SSA3 and ERV2; SSA3 and EUG1; SSA3 and MPD1; SSA3 and MPD2; SSA3 and EPS1; SSA3 and PDI1; SSA3 and DER1; SSA3 and DER3; SSA3 and HRD3; SSA3 and UBC7; SSA3 and HAC1; SSA4 and SSE1; SSA4 and SSE2; SSA4 and SSB1; SSA4 and SSB2; SSA4 and ECM10; SSA4 and MDJ1; SSA4 and MDJ2; SSA4 and ERO1; SSA4 and ERV2; SSA4 and EUG1; SSA4 and MPD1; SSA4 and MPD2; SSA4 and EPS1; SSA4 and PDI1; SSA4 and DER1; SSA4 and DER3; SSA4 and HRD3; SSA4 and UBC7; SSA4 and HAC1; SSE1 and SSE2; SSE1 and SSB1; SSE1 and SSB2; SSE1 and ECM10; SSE1 and MDJ1; SSE1 and MDJ2; SSE1 and ERO1; SSE1 and ERV2; SSE1 and EUG1; SSE1 and MPD1; SSE1 and MPD2; SSE1 and EPS1; SSE1 and PDI1; SSE1 and DER1; SSE1 and DER3; SSE1 and HRD3; SSE1 and UBC7; SSE1 and HAC1; SSE2 and SSB1; SSE2 and SSB2; SSE2 and ECM10; SSE2 and MDJ1; SSE2 and MDJ2; SSE2 and ERO1; SSE2 and ERV2; SSE2 and EUG1; SSE2 and MPD1; SSE2 and MPD2; SSE2 and EPS1; SSE2 and PDI1; SSE2 and DER1; SSE2 and DER3; SSE2 and HRD3; SSE2 and UBC7; SSE2 and HAC1; SSB1 and SSB2; SSB1 and ECM10; SSB1 and MDJ1; SSB1 and MDJ2; SSB1 and ERO1; SSB1 and ERV2; SSB1 and EUG1; SSB1 and MPD1; SSB1 and MPD2; SSB1 and EPS1; SSB1 and PDI1; SSB1 and DER1; SSB1 and DER3; SSB1 and HRD3; SSB1 and UBC7; SSB1 and HAC1; SSB2 and ECM10; SSB2 and MDJ1; SSB2 and MDJ2; SSB2 and ERO1; SSB2 and ERV2; SSB2 and EUG1; SSB2 and MPD1; SSB2 and MPD2; SSB2 and EPS1; SSB2 and PDI1; SSB2 and DER1; SSB2 and DER3; SSB2 and HRD3; SSB2 and UBC7; SSB2 and HAC1; ECM10 and MDJ1; ECM10 and MDJ2; ECM10 and ERO1; ECM10 and ERV2; ECM10 and EUG1; ECM10 and MPD1; ECM10 and MPD2; ECM10 and EPS1; ECM10 and PDI1; ECM10 and DER1; ECM10 and DER3; ECM10 and HRD3; ECM10 and UBC7; ECM10 and HAC1; MDJ1 and MDJ2; MDJ1 and ERO1; MDJ1 and ERV2; MDJ1 and EUG1; MDJ1 and MPD1; MDJ1 and MPD2; MDJ1 and EPS1; MDJ1 and PDI1; MDJ1 and DER1; MDJ1 and DER3; MDJ1 and HRD3; MDJ1 and UBC7; MDJ1 and HAC1; MDJ2 and ERO1; MDJ2 and ERV2; MDJ2 and EUG1; MDJ2 and MPD1; MDJ2 and MPD2; MDJ2 and EPS1; MDJ2 and PDI1; MDJ2 and DER1; MDJ2 and DER3; MDJ2 and HRD3; MDJ2 and UBC7; MDJ2 and HAC1; ERO1 and ERV2; ERO1 and EUG1; ERO1 and MPD1; ERO1 and MPD2; ERO1 and EPS1; ERO1 and PDI1; ERO1 and DER1; ERO1 and DER3; ERO1 and HRD3; ERO1 and UBC7; ERO1 and HAC1; ERV2 and EUG1; ERV2 and MPD1; ERV2 and MPD2; ERV2 and EPS1; ERV2 and PDI1; ERV2 and DER1; ERV2 and DER3; ERV2 and HRD3; ERV2 and UBC7; ERV2 and HAC1; EUG1 and MPD1; EUG1 and MPD2; EUG1 and EPS1; EUG1 and PDI1; EUG1 and DER1; EUG1 and DER3; EUG1 and HRD3; EUG1 and UBC7; EUG1 and HAC1; MPD1 and MPD2; MPD1 and EPS1; MPD1 and PDI1; MPD1 and DER1; MPD1 and DER3; MPD1 and HRD3; MPD1 and UBC7; MPD1 and HAC1; MPD2 and EPS1; MPD2 and PDI1; MPD2 and DER1; MPD2 and DER3; MPD2 and HRD3; MPD2 and UBC7; MPD2 and HAC1; EPS1 and PDI1; EPS1 and DER1; EPS1 and DER3; EPS1 and HRD3; EPS1 and UBC7; EPS1 and HAC1; PDI1 and DER1; PDI1 and DER3; PDI1 and HRD3; PDI1 and UBC7; PDI1 and HAC1; DER1 and DER3; DER1 and HRD3; DER1 and UBC7; DER1 and HAC1; DER3 and HRD3; DER3 and UBC7; DER3 and HAC1; HRD3 and UBC7; HRD3 and HAC1; or UBC7 and HAC1.

HAC1 in combination with any one of the following combinations: JEM1 and LHS1; JEM1 and SCJ1; JEM1 and KAR2; JEM1 and SELL JEM1 and FKB2; JEM1 and SSA1; JEM1 and SSA2; JEM1 and SSA3; JEM1 and SSA4; JEM1 and SSE1; JEM1 and SSE2; JEM1 and SSB1; JEM1 and SSB2; JEM1 and ECM10; JEM1 and MDJ1; JEM1 and MDJ2; JEM1 and ERO1; JEM1 and ERV2; JEM1 and EUG1; JEM1 and MPD1; JEM1 and MPD2; JEM1 and EPS1; JEM1 and PDI1; JEM1 and DER1; JEM1 and DER3; JEM1 and HRD3; JEM1 and UBC7; JEM1 and DOA4; LHS1 and SCJ1; LHS1 and KAR2; LHS1 and SIL1 LHS1 and FKB2; LHS1 and SSA1; LHS1 and SSA2; LHS1 and SSA3; LHS1 and SSA4; LHS1 and SSE1; LHS1 and SSE2; LHS1 and SSB1; LHS1 and SSB2; LHS1 and ECM10; LHS1 and MDJ1; LHS1 and MDJ2; LHS1 and ERO1; LHS1 and ERV2; LHS1 and EUG1; LHS1 and MPD1; LHS1 and MPD2; LHS1 and EPS1; LHS1 and PDI1; LHS1 and DER1; LHS1 and DER3; LHS1 and HRD3; LHS1 and UBC7; LHS1 and DOA4; SCJ1 and KAR2; SCJ1 and SIL1; SCJ1 and FKB2; SCJ1 and SSA1; SCJ1 and SSA2; SCJ1 and SSA3; SCJ1 and SSA4; SCJ1 and SSE1; SCJ1 and SSE2; SCJ1 and SSB1; SCJ1 and SSB2; SCJ1 and ECM10; SCJ1 and MDJ1; SCJ1 and MDJ2; SCJ1 and ERO1; SCJ1 and ERV2; SCJ1 and EUG1; SCJ1 and MPD1; SCJ1 and MPD2; SCJ1 and EPS1; SCJ1 and PDI1; SCJ1 and DER1; SCJ1 and DER3; SCJ1 and HRD3; SCJ1 and UBC7; SCJ1 and DOA4; KAR2 and SIL1; KAR2 and FKB2; KAR2 and SSA1; KAR2 and SSA2; KAR2 and SSA3; KAR2 and SSA4; KAR2 and SSE1; KAR2 and SSE2; KAR2 and SSB1; KAR2 and SSB2; KAR2 and ECM10; KAR2 and MDJ1; KAR2 and MDJ2; KAR2 and ERO1; KAR2 and ERV2; KAR2 and EUG1; KAR2 and MPD1; KAR2 and MPD2; KAR2 and EPS1; KAR2 and PDI1; KAR2 and DER1; KAR2 and DER3; KAR2 and HRD3; KAR2 and UBC7; KAR2 and DOA4; SIL1 and FKB2; SIL1 and SSA1; SIL1 and SSA2; SIL1 and SSA3; SIL1 and SSA4; SIL1 and SSE1; SIL1 and SSE2; SIL1 and SSB1; SIL1 and SSB2; SIL1 and ECM10; SIL1 and MDJ1; SIL1 and MDJ2; SIL1 and ERO1; SIL1 and ERV2; SIL1 and EUG1; SIL1 and MPD1; SIL1 and MPD2; SIL1 and EPS1; SIL1 and PDI1; SIL1 and DER1; SIL1 and DER3; SIL1 and HRD3; SIL1 and UBC7; SIL1 and DOA4; FKB2 and SSA1; FKB2 and SSA2; FKB2 and SSA3; FKB2 and SSA4; FKB2 and SSE1; FKB2 and SSE2; FKB2 and SSB1; FKB2 and SSB2; FKB2 and ECM10; FKB2 and MDJ1; FKB2 and MDJ2; FKB2 and ERO1; FKB2 and ERV2; FKB2 and EUG1; FKB2 and MPD1; FKB2 and MPD2; FKB2 and EPS1; FKB2 and PDI1; FKB2 and DER1; FKB2 and DER3; FKB2 and HRD3; FKB2 and UBC7; FKB2 and DOA4; SSA1 and SSA2; SSA1 and SSA3; SSA1 and SSA4; SSA1 and SSE1; SSA1 and SSE2; SSA1 and SSB1; SSA1 and SSB2; SSA1 and ECM10; SSA1 and MDJ1; SSA1 and MDJ2; SSA1 and ERO1; SSA1 and ERV2; SSA1 and EUG1; SSA1 and MPD1; SSA1 and MPD2; SSA1 and EPS1; SSA1 and PDI1; SSA1 and DER1; SSA1 and DER3; SSA1 and HRD3; SSA1 and UBC7; SSA1 and DOA4; SSA2 and SSA3; SSA2 and SSA4; SSA2 and SSE1; SSA2 and SSE2; SSA2 and SSB1; SSA2 and SSB2; SSA2 and ECM10; SSA2 and MDJ1; SSA2 and MDJ2; SSA2 and ERO1; SSA2 and ERV2; SSA2 and EUG1; SSA2 and MPD1; SSA2 and MPD2; SSA2 and EPS1; SSA2 and PDI1; SSA2 and DER1; SSA2 and DER3; SSA2 and HRD3; SSA2 and UBC7; SSA2 and DOA4; SSA3 and SSA4; SSA3 and SSE1; SSA3 and SSE2; SSA3 and SSB1; SSA3 and SSB2; SSA3 and ECM10; SSA3 and MDJ1; SSA3 and MDJ2; SSA3 and ERO1; SSA3 and ERV2; SSA3 and EUG1; SSA3 and MPD1; SSA3 and MPD2; SSA3 and EPS1; SSA3 and PDI1; SSA3 and DER1; SSA3 and DER3; SSA3 and HRD3; SSA3 and UBC7; SSA3 and DOA4; SSA4 and SSE1; SSA4 and SSE2; SSA4 and SSB1; SSA4 and SSB2; SSA4 and ECM10; SSA4 and MDJ1; SSA4 and MDJ2; SSA4 and ERO1; SSA4 and ERV2; SSA4 and EUG1; SSA4 and MPD1; SSA4 and MPD2; SSA4 and EPS1; SSA4 and PDI1; SSA4 and DER1; SSA4 and DER3; SSA4 and HRD3; SSA4 and UBC7; SSA4 and DOA4; SSE1 and SSE2; SSE1 and SSB1; SSE1 and SSB2; SSE1 and ECM10; SSE1 and MDJ1; SSE1 and MDJ2; SSE1 and ERO1; SSE1 and ERV2; SSE1 and EUG1; SSE1 and MPD1; SSE1 and MPD2; SSE1 and EPS1; SSE1 and PDI1; SSE1 and DER1; SSE1 and DER3; SSE1 and HRD3; SSE1 and UBC7; SSE1 and DOA4; SSE2 and SSB1; SSE2 and SSB2; SSE2 and ECM10; SSE2 and MDJ1; SSE2 and MDJ2; SSE2 and ERO1; SSE2 and ERV2; SSE2 and EUG1; SSE2 and MPD1; SSE2 and MPD2; SSE2 and EPS1; SSE2 and PDI1; SSE2 and DER1; SSE2 and DER3; SSE2 and HRD3; SSE2 and UBC7; SSE2 and DOA4; SSB1 and SSB2; SSB1 and ECM10; SSB1 and MDJ1; SSB1 and MDJ2; SSB1 and ERO1; SSB1 and ERV2; SSB1 and EUG1; SSB1 and MPD1; SSB1 and MPD2; SSB1 and EPS1; SSB1 and PDI1; SSB1 and DER1; SSB1 and DER3; SSB1 and HRD3; SSB1 and UBC7; SSB1 and DOA4; SSB2 and ECM10; SSB2 and MDJ1; SSB2 and MDJ2; SSB2 and ERO1; SSB2 and ERV2; SSB2 and EUG1; SSB2 and MPD1; SSB2 and MPD2; SSB2 and EPS1; SSB2 and PDI1; SSB2 and DER1; SSB2 and DER3; SSB2 and HRD3; SSB2 and UBC7; SSB2 and DOA4; ECM10 and MDJ1; ECM10 and MDJ2; ECM10 and ERO1; ECM10 and ERV2; ECM10 and EUG1; ECM10 and MPD1; ECM10 and MPD2; ECM10 and EPS1; ECM10 and PDI1; ECM10 and DER1; ECM10 and DER3; ECM10 and HRD3; ECM10 and UBC7; ECM10 and DOA4; MDJ1 and MDJ2; MDJ1 and ERO1; MDJ1 and ERV2; MDJ1 and EUG1; MDJ1 and MPD1; MDJ1 and MPD2; MDJ1 and EPS1; MDJ1 and PDI1; MDJ1 and DER1; MDJ1 and DER3; MDJ1 and HRD3; MDJ1 and UBC7; MDJ1 and DOA4; MDJ2 and ERO1; MDJ2 and ERV2; MDJ2 and EUG1; MDJ2 and MPD1; MDJ2 and MPD2; MDJ2 and EPS1; MDJ2 and PDI1; MDJ2 and DER1; MDJ2 and DER3; MDJ2 and HRD3; MDJ2 and UBC7; MDJ2 and DOA4; ERO1 and ERV2; ERO1 and EUG1; ERO1 and MPD1; ERO1 and MPD2; ERO1 and EPS1; ERO1 and PDI1; ERO1 and DER1; ERO1 and DER3; ERO1 and HRD3; ERO1 and UBC7; ERO1 and DOA4; ERV2 and EUG1; ERV2 and MPD1; ERV2 and MPD2; ERV2 and EPS1; ERV2 and PDI1; ERV2 and DER1; ERV2 and DER3; ERV2 and HRD3; ERV2 and UBC7; ERV2 and DOA4; EUG1 and MPD1; EUG1 and MPD2; EUG1 and EPS1; EUG1 and PDI1; EUG1 and DER1; EUG1 and DER3; EUG1 and HRD3; EUG1 and UBC7; EUG1 and DOA4; MPD1 and MPD2; MPD1 and EPS1; MPD1 and PDI1; MPD1 and DER1; MPD1 and DER3; MPD1 and HRD3; MPD1 and UBC7; MPD1 and DOA4; MPD2 and EPS1; MPD2 and PDI1; MPD2 and DER1; MPD2 and DER3; MPD2 and HRD3; MPD2 and UBC7; MPD2 and DOA4; EPS1 and PDI1; EPS1 and DER1; EPS1 and DER3; EPS1 and HRD3; EPS1 and UBC7; EPS1 and DOA4; PDI1 and DER1; PDI1 and DER3; PDI1 and HRD3; PDI1 and UBC7; PDI1 and DOA4; DER1 and DER3; DER1 and HRD3; DER1 and UBC7; DER1 and DOA4; DER3 and HRD3; DER3 and UBC7; DER3 and DOA4; HRD3 and UBC7; HRD3 and DOA4; or UBC7 and DOA4.

### Protein Product of Choice

In principle, any protein can be expressed as the protein product of choice.

As discussed above, the protein product of choice may or may not be a protein that is naturally produced by the host cell, in which case the protein may or may not be encoded by the host cell's endogenous gene for that protein or the protein may or may not be encoded (fully, or in part) by an exogenous polynucleotide sequence.

Thus, it is possible to produce enhanced levels of naturally produced proteins by transforming the host cell with a polynucleotide encoding a further, or replacement, copy of an endogenous gene, or otherwise genetically modifying the host cell to increase the expression of a naturally produced protein. In one embodiment, a recombinant or genetically modified endogenous gene has a sequence that is different to the endogenous genetic material of the host cell.

The protein product of choice may or may not be a heterologous protein, by which we mean that the protein is one that is not naturally produced by the host cell. In the case of a heterologous protein product of choice, the protein may or may not be encoded by an exogenous polynucleotide sequence.

In one embodiment, the protein product of choice is secreted. In that case, a sequence encoding a secretion leader sequence which, for example, comprises most of the natural HSA secretion leader, plus a small portion of the S. cerevisiae α-mating factor secretion leader as taught in WO 90/01063 may or may not be included in the open reading frame that encodes the protein product of choice.

Alternatively, the protein product of choice may or may not be intracellular.

It is known in the prior art that enhanced protein production can be achieved by co-expression of a protein product and a chaperone in different compartments of the cell. For example, WO 2005/061718 (Example 12) describes the co-over-expression of the cytoplasmic chaperone SSA1 and a secreted recombinant transferrin, in order to increase the production of the secreted recombinant transferrin.

In another preferred embodiment, the protein product of choice comprises the sequence of a eukaryotic protein, or a fragment or variant thereof. Suitable eukaryotes include fungi, plants and animals. In one preferred embodiment the protein product of choice is a fungal protein, such as a yeast protein. In another preferred embodiment the protein product of choice is an animal protein. Exemplary animals include vertebrates and invertebrates. Exemplary vertebrates include mammals, such as humans, and non-human mammals.

Thus the protein product of choice may or may not comprise the sequence of a yeast protein.

The protein product of choice may or may not comprise albumin, a monoclonal antibody, an etoposide, a serum protein (such as a blood clotting factor), antistasin, a tick anticoagulant peptide, transferrin, lactoferrin, endostatin, angiostatin, collagens, immunoglobulins or immunoglobulin-based-molecules or fragment of either (e.g. a Small Modular ImmunoPharmaceutical™ (“SMTP”) or dAb, Fab′ fragments, F(ab′)₂, scAb, scFv or scFv fragment), a Kunitz domain protein (such as aprotinin, amyloid precursor protein and those described in WO 03/066824, with or without albumin fusions), interferons (such as interferon α species and sub-species, interferon (3 species and sub-species, interferon γ species and sub-species), interleukins (such as IL10, IL11 and IL2), leptin, CNTF and fragment thereof (such as CNTF_{Ax15} (Axokine™)), IL1-receptor antagonist, erythropoietin (EPO) and EPO mimics, thrombopoietin (TPO) and TPO mimics, prosaptide, cyanovirin-N, 5-helix, T20 peptide, T1249 peptide, HIV gp41, HIV gp120, urokinase, prourokinase, tPA, hirudin, platelet derived growth factor, parathyroid hormone, proinsulin, insulin, glucagon, glucagon-like peptides, insulin-like growth factor, calcitonin, growth hormone, transforming growth factor β, tumour necrosis factor, G-CSF, GM-CSF, M-CSF, FGF, coagulation factors in both pre and active forms, including but not limited to plasminogen, fibrinogen, thrombin, pre-thrombin, pro-thrombin, von Willebrand's factor, α₁-antitrypsin, plasminogen activators, Factor VII, Factor Vac Factor IX, Factor X and Factor Mil, nerve growth factor, LACI, platelet-derived endothelial cell growth factor (PD-ECGF), glucose oxidase, serum cholinesterase, inter-alpha trypsin inhibitor, antithrombin III, apo-lipoprotein species, Protein C, Protein S, or a variant or fragment of any of the above.

A “variant”, in the context of the above-listed proteins, refers to a protein wherein at one or more positions there have been amino acid insertions, deletions, or substitutions, either conservative or non-conservative, provided that such changes result in a protein whose basic properties, for example enzymatic activity or receptor binding (type of and specific activity), thermostability, activity in a certain pH-range (pH-stability) have not significantly been changed. “Significantly” in this context means that one skilled in the art would say that the properties of the variant may still be different but would not be unobvious over the ones of the original protein.

By “conservative substitutions” is intended combinations such as Val, Ile, Leu, Ala, Met; Asp, Glu; Asn, Gln; Ser, Thr, Gly, Ala; Lys, Arg, His; and Phe, Tyr, Trp. Preferred conservative substitutions include Gly, Ala; Val, Ile, Leu; Asp, Glu; Asn, Gln; Ser, Thr; Lys, Arg; and Phe, Tyr.

A “variant” typically has at least 25%, at least 50%, at least 60% or at least 70%, preferably at least 80%, more preferably at least 90%, even more preferably at least 95%, yet more preferably at least 99%, most preferably at least 99.5% sequence identity to the polypeptide from which it is derived.

The percent sequence identity between two polypeptides may be determined using suitable computer programs, for example the GAP program of the University of Wisconsin Genetic Computing Group and it will be appreciated that percent identity is calculated in relation to polypeptides whose sequence has been aligned optimally.

The alignment may alternatively be carried out using the Clustal W program (Thompson et al., (1994) Nucleic Acids Res., 22(22), 4673-80). The parameters used may be as follows:
Fast pairwise alignment parameters: K-tuple(word) size; 1, window size; 5, gap penalty; 3, number of top diagonals; 5. Scoring method: x percent.
Multiple alignment parameters: gap open penalty; 10, gap extension penalty; 0.05.
Scoring matrix: BLOSUM.

Such variants may or may not be natural or made using the methods of protein engineering and site-directed mutagenesis as are well known in the art.

A “fragment”, in the context of the above-listed proteins, refers to a protein wherein at one or more positions there have been deletions. Thus the fragment may comprise at most 5, 10, 20, 30, 40 or 50% of the complete sequence of the full mature polypeptide. Typically a fragment comprises up to 60%, more typically up to 70%, preferably up to 80%, more preferably up to 90%, even more preferably up to 95%, yet more preferably up to 99% of the complete sequence of the full desired protein. Particularly preferred fragments of a protein comprise one or more whole domains of the protein.

In one particularly preferred embodiment the protein product of choice comprises the sequence of albumin or a variant or fragment thereof.

By “albumin” we include a protein comprising the sequence of an albumin protein obtained from any source. Typically the source is mammalian. In one preferred embodiment the serum albumin is human serum albumin (“HSA”). The term “human serum albumin” includes the meaning of a serum albumin having an amino acid sequence naturally occurring in humans, and variants thereof. Preferably the albumin has the amino acid sequence disclosed in WO 90/13653 or a variant thereof. The HSA coding sequence is obtainable by known methods for isolating cDNA corresponding to human genes, and is also disclosed in, for example, EP 73 646 and EP 286 424.

In another preferred embodiment the “albumin” comprises the sequence of bovine serum albumin. The term “bovine serum albumin” includes the meaning of a serum albumin having an amino acid sequence naturally occurring in cows, for example as taken from Swissprot accession number P02769, and variants thereof as defined below. The term “bovine serum albumin” also includes the meaning of fragments, of full-length bovine serum albumin or variants thereof, as defined below.

In another preferred embodiment the albumin comprises the sequence of an albumin derived from one of serum albumin from dog (e.g. see Swissprot accession number P49822), pig (e.g. see Swissprot accession number P08835), goat (e.g. as available from Sigma as product no. A2514 or A4164), turkey (e.g. see Swissprot accession number O73860), baboon (e.g. as available from Sigma as product no. A1516), cat (e.g. see Swissprot accession number P49064), chicken (e.g. see Swissprot accession number P19121), ovalbumin (e.g. chicken ovalbumin) (e.g. see Swissprot accession number P01012), donkey (e.g. see Swissprot accession number P39090), guinea pig (e.g. as available from Sigma as product no. A3060, A2639, O5483 or A6539), hamster (e.g. as available from Sigma as product no. A5409), horse (e.g. see Swissprot accession number P35747), rhesus monkey (e.g. see Swissprot accession number Q28522), mouse (e.g. see Swissprot accession number O89020), pigeon (e.g. as defined by Khan et al, 2002, Int. J. Biol. Macromol., 30(3-4)371-8), rabbit (e.g. see Swissprot accession number P49065), rat (e.g. see Swissprot accession number P36953) and sheep (e.g. see Swissprot accession number P14639) and includes variants and fragments thereof as defined below.

Many naturally occurring mutant forms of albumin are known. Many are described in Peters, (1996, All About Albumin: Biochemistry, Genetics and Medical Applications, Academic Press, Inc., San Diego, Calif., p. 170-181). A variant as defined above may or may not be one of these naturally occurring mutants.

A “variant albumin” refers to an albumin protein wherein at one or more positions there have been amino acid insertions, deletions, or substitutions, either conservative or non-conservative, provided that such changes result in an albumin protein for which at least one basic property, for example binding activity (type of and specific activity e.g. binding to bilirubin), osmolarity (oncotic pressure, colloid osmotic pressure), behaviour in a certain pH-range (pH-stability) has not significantly been changed. “Significantly” in this context means that one skilled in the art would say that the properties of the variant may still be different but would not be unobvious over the ones of the original protein.

By “conservative substitutions” is intended combinations such as Gly, Ala; Val, Ile, Leu; Asp, Glu; Asn, Gln; Ser, Thr, Lys, Arg; and Phe, Tyr. Such variants may be made by techniques well known in the art, such as by site-directed mutagenesis as disclosed in U.S. Pat. No. 4,302,386 issued 24 Nov. 1981 to Stevens, incorporated herein by reference.

Typically an albumin variant will have more than 40%, usually at least 50%, more typically at least 60%, preferably at least 70%, more preferably at least 80%, yet more preferably at least 90%, even more preferably at least 95%, most preferably at least 98% or more sequence identity with naturally occurring albumin. The percent sequence identity between two polypeptides may be determined using suitable computer programs, for example the GAP program of the University of Wisconsin Genetic Computing Group and it will, be appreciated that percent identity is calculated in relation to polypeptides whose sequence has been aligned optimally. The alignment may alternatively be carried out using the Clustal W program (Thompson et al., 1994). The parameters used may be as follows:

Fast pairwise alignment parameters: K-tuple(word) size; 1, window size; 5, gap penalty; 3, number of top diagonals; 5. Scoring method: x percent. Multiple alignment parameters: gap open penalty; 10, gap extension penalty; 0.05. Scoring matrix: BLOSUM.

The term “fragment” as used above includes any fragment of full-length albumin or a variant thereof, so long as at least one basic property, for example binding activity (type of and specific activity e.g. binding to bilirubin), osmolarity (oncotic pressure, colloid osmotic pressure), behaviour in a certain pH-range (pH-stability) has not significantly been changed. “Significantly” in this context means that one skilled in the art would say that the properties of the variant may still be different but would not be unobvious over the ones of the original protein. A fragment will typically be at least 50 amino acids long. A fragment may or may not comprise at least one whole sub-domain of albumin Domains of HSA have been expressed as recombinant proteins (Dockal, M. et al., 1999, J. Biol. Chem., 274, 29303-29310), where domain I was defined as consisting of amino acids 1-197, domain II was defined as consisting of amino acids 189-385 and domain III was defined as consisting of amino acids 381-585. Partial overlap of the domains occurs because of the extended α-helix structure (h10-h1) which exists between domains I and II, and between domains II and III (Peters, 1996, op. cit., Table 2-4). HSA also comprises six sub-domains (sub-domains IA, IB, IIA, IIB, IIIA and IIIB). Sub-domain IA comprises amino acids 6-105, sub-domain IB comprises amino acids 120-177, sub-domain IIA comprises amino acids 200-291, sub-domain IIB comprises amino acids 316-369, sub-domain IIIA comprises amino acids 392-491 and sub-domain IIIB comprises amino acids 512-583. A fragment may or may not comprise a whole or part of one or more domains or sub-domains as defined above, or any combination of those domains and/or sub-domains.

In another particularly preferred embodiment the protein product of choice comprises the sequence of transferrin or a variant or fragment thereof. The term “transferrin” as used herein includes all members of the transferrin family (Testa, Proteins of iron metabolism, CRC Press, 2002; Harris & Aisen, Iron carriers and iron proteins, Vol. 5, Physical Bioinorganic Chemistry, VCH, 1991) and their derivatives, such as transferrin, mutant transferrins (Mason et al, 1993, Biochemistry, 32, 5472; Mason et al, 1998, Biochem. J., 330(1), 35), truncated transferrins, transferrin lobes (Mason et al, 1996, Protein Expr. Purif., 8, 119; Mason et al, 1991, Protein Expr. Purif., 2, 214), lactoferrin, mutant lactoferrins, truncated lactoferrins, lactoferrin lobes or fusions of any of the above to other peptides, polypeptides or proteins (Shin et al, 1995, Proc. Natl. Acad Sci. USA, 92, 2820; Ali et al, 1999, J. Biol. Chem., 274, 24066; Mason et al, 2002, Biochemistry, 41, 9448).

The transferrin may or may not be human transferrin. The term “human transferrin” is used herein to denote material which is indistinguishable from transferrin derived from a human or which is a variant or fragment thereof. A “variant” includes insertions, deletions and substitutions, either conservative or non-conservative, where such changes do not substantially alter the useful ligand-binding or immunogenic properties of transferrin.

Mutants of transferrin are included in the invention. Such mutants may or may not have altered immunogenicity. For example, transferrin mutants may or may not display modified (e.g. reduced) glycosylation. The N-linked glycosylation pattern of a transferrin molecule can be modified by adding/removing amino acid glycosylation consensus sequences such as N-X-S/T, at any or all of the N, X, or S/T position. Transferrin mutants may or may not be altered in their natural binding to metal ions and/or other proteins, such as transferrin receptor. An example of a transferrin mutant modified in this manner is exemplified below.

We also include naturally-occurring polymorphic variants of human transferrin or human transferrin analogues. Generally, variants or fragments of human transferrin will have at least 5%, 10%, 15%, 20%, 30%, 40% or 50% (preferably at least 80%, 90% or 95%) of human transferrin's ligand binding activity (for example iron-binding), weight for weight. The iron binding activity of transferrin or a test sample can be determined spectrophotometrically by 470 nm:280 nm absorbance ratios for the proteins in their iron-free and fully iron-loaded states. Reagents should be iron-free unless stated otherwise. Iron can be removed from transferrin or the test sample by dialysis against 0.1M citrate, 0.1M acetate, 10 mM EDTA pH4.5. Protein should be at approximately 20 mg/mL in 100 mM HEPES, 10 mM NaHCO₃ pH8.0. Measure the 470 nm:280 nm absorbance ratio of apo-transferrin (Calbiochem, CN Biosciences, Nottingham, UK) diluted in water so that absorbance at 280 nm can be accurately determined spectrophotometrically (0% iron binding). Prepare 20 mM iron-nitrilotriacetate (FeNTA) solution by dissolving 191 mg nitrotriacetic acid in 2 mT, 1M NaOH, then add 2 mL 0.5M ferric chloride. Dilute to 50 mL with deionised water. Fully load apo-transferrin with iron (100% iron binding) by adding a sufficient excess of freshly prepared 20 mM FeNTA, then dialyse the holo-transferrin preparation completely against 100 mM HEPES, 10 mM NaHCO₃ pH8.0 to remove remaining FeNTA before measuring the absorbance ratio at 470 nm:280 nm. Repeat the procedure using test sample, which should initially be free from iron, and compare final ratios to the control. Additionally, single or multiple heterologous fusions comprising any of the above; or single or multiple heterologous fusions to albumin, transferrin or immunoglobulins or a variant or fragment of any of these may be used. Such fusions include albumin N-terminal fusions, albumin C-terminal fusions and co-N-terminal and C-terminal albumin fusions as exemplified by WO 01/79271, and transferrin N-terminal fusions, transferrin C-terminal fusions, and co-N-terminal and C-terminal transferrin fusions.

Examples of transferrin fusions are given in US patent applications US2003/0221201 and US2003/0226155, Shin, et al., 1995, Proc Natl Acad Sci USA, 92, 2820, Ali, et al., 1999, J Biol Chem, 274, 24066, Mason, et al., 2002, Biochemistry, 41, 9448, the contents of which are incorporated herein by reference.

The skilled person will also appreciate that the open reading frame of any other gene or variant, or part or either, can be utilised as an open reading frame for use with the present invention. For example, the open reading frame may encode a protein comprising any sequence, be it a natural protein (including a zymogen), or a variant, or a fragment (which may or may not, for example, be a domain) of a natural protein; or a totally synthetic protein; or a single or multiple fusion of different proteins (natural or synthetic). Such proteins can be taken, but not exclusively, from the lists provided in WO 01/79258, WO 01/79271, WO 01/79442, WO 01/79443, WO 01/79444 and WO 01/79480, or a variant or fragment thereof; the disclosures of which are incorporated herein by reference. Although these patent applications present the list of proteins in the context of fusion partners for albumin, the present invention is not so limited and, for the purposes of the present invention, any of the proteins listed therein may be presented alone or as fusion partners for albumin, the Fc region of immunoglobulin, transferrin, lactoferrin or any other protein or fragment or variant of any of the above, as a desired polypeptide.

The protein product of choice may or may not be a therapeutically active protein. In other words, it may or may not have a recognised medical effect on individuals, such as humans. Many different types of therapeutically active protein are well known in the art.

As discussed above, the protein product of choice may or may not comprise a leader sequence effective to cause secretion in the host cell (such as in a yeast host cell).

Numerous natural or artificial polypeptide signal sequences (also called secretion pre regions) have been used or developed for secreting proteins from host cells. The signal sequence directs the nascent protein towards the machinery of the cell that exports proteins from the cell into the surrounding medium or, in some cases, into the periplasmic space. The signal sequence is usually, although not necessarily, located at the N-terminus of the primary translation product and is generally, although not necessarily, cleaved off the protein during the secretion process, to yield the “mature” protein.

In the case of some proteins the entity that is initially secreted, after the removal of the signal sequence, includes additional amino acids at its N-terminus called a “pro” sequence, the intermediate entity being called a “pro-protein”. These pro sequences may assist the final protein to fold and become functional, and are usually then cleaved off. In other instances, the pro region simply provides a cleavage site for an enzyme to cleave off the pre-pro region and is not known to have another function.

The pro sequence can be removed either during the secretion of the protein from the cell or after export from the cell into the surrounding medium or periplasmic space.

Polypeptide sequences which direct the secretion of proteins, whether they resemble signal (i.e. pre) sequences or pre-pro secretion sequences, are referred to as leader sequences. The secretion of proteins is a dynamic process involving translation, translocation and post-translational processing, and one or more of these steps may not necessarily be completed before another is either initiated or completed.

For production of proteins in eukaryotic species such as the yeasts Saccharomyces cerevisiae, Zygosaccharomyces species, Kluyveromyces lactis and Pichia pastoris, known leader sequences include those from the S. cerevisiae acid phosphatase protein (Pho5p) (see EP 366 400), the invertase protein (Suc2p) (see Smith et al. (1985) Science, 229, 1219-1224) and heat-shock protein-150 (Hsp150p) (see WO 95/33833). Additionally, leader sequences from the S. cerevisiae mating factor alpha-1 protein (MFα-1) and from the human lysozyme and human serum albumin (HSA) protein have been used, the latter having been used especially, although not exclusively, for secreting human albumin. WO 90/01063 discloses a fusion of the MFα-1 and HSA leader sequences, which advantageously reduces the production of a contaminating fragment of human albumin relative to the use of the MFα-1 leader sequence. Modified leader sequences are also disclosed in the examples of this application and the reader will appreciate that those leader sequences can be used with proteins other than transferrin. In addition, the natural transferrin leader sequence may or may not be used to direct secretion of transferrin and other protein products of choice.

Where a helper protein is a chaperone involved in the formation of disulphide bonds, then in one embodiment the protein product of choice comprises disulphide bonds in its mature form. The disulphide bonds may be intramolecular and/or intermolecular.

The protein product of choice may or may not be a commercially useful protein. Some heterologously expressed proteins are intended to interact with the cell in which they are expressed in order to bring about a beneficial effect on the cell's activities. These proteins are not, in their own right, commercially useful. Commercially useful proteins are proteins that have a utility ex vivo of the cell in which they are expressed. Nevertheless, the skilled reader will appreciate that a commercially useful protein may or may not also have a biological effect on the host cell expressing it as a protein, but that that effect is not the main or sole reason for expressing the protein therein.

### Suitable Host Cells for the Practice of the Present Invention

The host cell may be any type of cell. The host cell may or may not be an animal (such as mammalian, avian, insect, etc.), plant, fungal or bacterial cell. Bacterial and fungal, such as yeast, host cells may or may not be preferred.

Thus, the host cell may or may not be an animal (such as mammalian, avian, insect, etc.) cell. Suitable methods for transformation of animal cells are well known in the art and include, for example the use of retrovirus vectors (such as lentivirus vectors). Wolkowicz et al, 2004, Methods Mol. Biol., 246, 391-411 describes the use of lentivirus vectors for delivery of recombinant nucleic acid sequences to mammalian cells for use in cell culture techniques. Fassler, 2004, EMBO Rep., 5(1), 28-9 reviews lentiviral transgene vectors and their use in the production of transgenic systems.

In one embodiment the host cell is a yeast cell, such as a member of the Saccharomyces, Kluyveromyces, or Pichia genus, such as Saccharomyces cerevisiae, Kluyveromyces lactis, Pichia pastoris and Pichia membranaefaciens, or Zygosaccharomyces rouxii, Zygosaccharomyces bailii, Zygosaccharomyces fermentati, Hansenula polymorpha (also known as Pichia angusta) or Kluyveromyces drosophilarum are preferred.

It may be particularly advantageous to use a yeast deficient in one or more protein mannosyl transferases involved in O-glycosylation of proteins, for instance by disruption of the gene coding sequence.

Recombinantly expressed proteins can be subject to undesirable post-translational modifications by the producing host cell. For example, the albumin protein sequence does not contain any sites for N-linked glycosylation and has not been reported to be modified, in nature, by O-linked glycosylation. However, it has been found that recombinant human albumin (“rHA”) produced in a number of yeast species can be modified by O-linked glycosylation, generally involving mannose. The mannosylated albumin is able to bind to the lectin Concanavalin A. The amount of mannosylated albumin produced by the yeast can be reduced by using a yeast strain deficient in one or more of the PMT genes (WO 94/04687). The most convenient way of achieving this is to create a yeast which has a defect in its genome such that a reduced level of one of the Pmt proteins is produced. For example, there may or may not be a deletion, insertion or transposition in the coding sequence or the regulatory regions (or in another gene regulating the expression of one of the PMT genes) such that little or no Pmt protein is produced. Alternatively, the yeast could be transformed to produce an anti-Pmt agent, such as an anti-Pmt antibody. Alternatively, the yeast could be cultured in the presence of a compound that inhibits the activity of one of the PMT genes (Duffy et al, “Inhibition of protein mannosyltransferase 1 (PMT1) activity in the pathogenic yeast Candida albicans”, International Conference on Molecular Mechanisms of Fungal Cell Wall Biogenesis, 26-31 Aug. 2001, Monte Verita, Switzerland, Poster Abstract P38; the poster abstract may be viewed at http://www.micro.biol.ethz.ch/cellwall/).

If a yeast other than S. cerevisiae is used, disruption of one or more of the genes equivalent to the PMT genes of S. cerevisiae is also beneficial, e.g. in Pichia pastoris or Kluyveromyces lactis. The sequence of PMT1 (or any other PMT gene) isolated from S. cerevisiae may be used for the identification or disruption of genes encoding similar enzymatic activities in other fungal species. The cloning of the PMT1 homologue of Kluyveromyces lactis is described in WO 94/04687.

The yeast may or may not also have a deletion of the HSP150 and/or YAP3 genes as taught respectively in WO 95/33833 and WO 95/23857.

Where one or more of the helper protein(s) and/or protein product of choice are encoded by a plasmid-borne polynucleotide sequence, the host cell type may be selected for compatibility with the plasmid type being used.

The skilled person will appreciate that any suitable plasmid may be used, such as a centromeric plasmid. The examples provide suitable plasmids (centromeric YCplac33-based vectors) for use to transform yeast host cells of the present invention. Alternatively, any other suitable plasmid may be used, such as a yeast-compatible 2 μm-based plasmid.

Plasmids obtained from one yeast type can be maintained in other yeast types (Irie et al, 1991, Gene, 108(1), 139-144; Irie et al, 1991, Mol. Gen. Genet., 225(2), 257-265). For example, pSR1 from Zygosaccharomyces rouxii can be maintained in Saccharomyces cerevisiae. In one embodiment the plasmid may or may not be a 2 μm-family plasmid and the host cell will be compatible with the 2 μm-family plasmid used (see below for a full description of the following plasmids). For example, where the plasmid is based on pSR1, pSB3 or pSB4 then a suitable yeast cell is Zygosaccharomyces rouxii; where the plasmid is based on pSB1 or pSB2 then a suitable yeast cell is Zygosaccharomyces bailli; where the plasmid is based on pSM1 then a suitable yeast cell is Zygosaccharomyces fermentati; where the plasmid is based on pKD1 then a suitable yeast cell is Kluyveromyces drosophilarum; where the plasmid is based on pPM1 then a suitable yeast cell is Pichia membranaefaciens; where the plasmid is based on the 2 μm plasmid then a suitable yeast cell is Saccharomyces cerevisiae or Saccharomyces carlsbergensis. Thus, the plasmid may be based on the 2 μm plasmid and the yeast cell may be Saccharomyces cerevisiae. A 2 μm-family plasmid can be said to be “based on” a naturally occurring plasmid if it comprises one, two or preferably three of the genes FLP, REP1 and REP2 having sequences derived from that naturally occurring plasmid.

A plasmid as defined above, may be introduced into a host through standard techniques. With regard to transformation of prokaryotic host cells, see, for example, Cohen et al (1972) Proc. Natl. Acad. Sci. USA 69, 2110 and Sambrook et al (2001) Molecular Cloning, A Laboratory Manual, 3^{rd} Ed. Cold Spring Harbor Laboratory, Cold Spring Harbor, N.Y. Transformation of yeast cells is described in Sherman et al (1986) Methods In Yeast Genetics, A Laboratory Manual, Cold Spring Harbor, N.Y. The method of Beggs (1978) Nature 275, 104-109 is also useful. Methods for the transformation of S. cerevisiae are taught generally in EP 251 744, EP 258 067 and WO 90/01063, all of which are incorporated herein by reference. With regard to vertebrate cells, reagents useful in transfecting such cells, for example calcium phosphate and DEAF-dextran or liposome formulations, are available from Stratagene Cloning Systems, or Life Technologies Inc., Gaithersburg, Md. 20877, USA.

Electroporation is also useful for transforming cells and is well known in the art for transforming fungal (including yeast) cell, plant cells, bacterial cells and animal (including vertebrate) cells. Methods for transformation of yeast by electroporation are disclosed in Becker & Guarente (1990) Methods Enzymol. 194, 182.

Generally, the plasmid will transform not all of the hosts and it will therefore be necessary to select for transformed host cells. Thus, a plasmid may comprise a selectable marker, including but not limited to bacterial selectable marker and/or a yeast selectable marker. A typical bacterial selectable marker is the β-lactamase gene although many others are known in the art. Typical yeast selectable marker include LEU2, TRP1, HIS3, HIS4, URA3, URA5, SFA1, ADE2, MET15, LYS5, LYS2, ILV2, FBA1, PSE1, PDI1 and PGK1. Those skilled in the art will appreciate that any gene whose chromosomal deletion or inactivation results in an unviable host, so called essential genes, can be used as a selective marker if a functional gene is provided on the plasmid, as demonstrated for PGK1 in a pgk1 yeast strain (Piper and Curran, 1990, Curr. Genet. 17, 119). Suitable essential genes can be found within the Stanford Genome Database (SGD), (http:://db.yeastgenome.org). Any essential gene product (e.g. PDI1, PSE1, PGK1 or FBA1) which, when deleted or inactivated, does not result in an auxotrophic (biosynthetic) requirement, can be used as a selectable marker on a plasmid in a host cell that, in the absence of the plasmid, is unable to produce that gene product, to achieve increased plasmid stability without the disadvantage of requiring the cell to be cultured under specific selective conditions. By “auxotrophic (biosynthetic) requirement” we include a deficiency which can be complemented by additions or modifications to the growth medium. Therefore, preferred “essential marker genes” in the context of the present application are those that, when deleted or inactivated in a host cell, result in a deficiency which cannot be complemented by additions or modifications to the growth medium. Additionally, a plasmid may comprise more than one selectable marker.

One selection technique involves incorporating into the expression vector a DNA sequence marker, with any necessary control elements, that codes for a selectable trait in the transformed cell. These markers include dihydrofolate reductase, G418, neomycin or zeocin resistance for eukaryotic cell culture, and tetracycline, kanamycin, ampicillin (i.e. β-lactamase) or zeocin resistance genes for culturing in E. coli and other bacteria. Zeocin resistance vectors are available from Invitrogen. Alternatively, the gene for such selectable trait can be on another vector, which is used to co-transform the desired host cell.

Another method of identifying successfully transformed cells involves growing the cells resulting from the introduction of a plasmid, optionally to allow the expression of a recombinant polypeptide (i.e. a polypeptide which is encoded by a polynucleotide sequence on the plasmid and is heterologous to the host cell, in the sense that that polypeptide is not naturally produced by the host). Cells can be harvested and lysed and their DNA or RNA content examined for the presence of the recombinant sequence using a method such as that described by Southern (1975) J. Mol. Biol. 98, 503 or Berent et al (1985) Biotech. 3, 208 or other methods of DNA and RNA analysis common in the art. Alternatively, the presence of a polypeptide in the supernatant of a culture of a transformed cell can be detected using antibodies.

In addition to directly assaying for the presence of recombinant DNA, successful transformation can be confirmed by well known immunological methods when the recombinant DNA is capable of directing the expression of the protein. For example, cells successfully transformed with an expression vector produce proteins displaying appropriate antigenicity. Samples of cells suspected of being transformed are harvested and assayed for the protein using suitable antibodies.

Thus, in addition to the transformed host cells themselves, the present invention also contemplates a culture of those cells, preferably a monoclonal (clonally homogeneous) culture, or a culture derived from a monoclonal culture, in a nutrient medium. Alternatively, transformed cells may represent an industrially/commercially or pharmaceutically useful product and can be used without further purification or can be purified from a culture medium and optionally formulated with a carrier or diluent in a manner appropriate to their intended industrial/commercial or pharmaceutical use, and optionally packaged and presented in a manner suitable for that use. For example, whole cells could be immobilised; or used to spray a cell culture directly on to/into a process, crop or other desired target. Similarly; whole cell, such as yeast cells can be used as capsules for a huge variety of applications, such as fragrances, flavours and pharmaceuticals.

Transformed host cells may be cultured for a sufficient time and under appropriate conditions known to those skilled in the art, and in view of the teachings disclosed herein, to permit the expression of the helper protein(s) and the protein product of choice.

The culture medium may be non-selective or place a selective pressure on the maintenance of a plasmid.

The thus produced protein product of choice may be present intracellularly or, if secreted, in the culture medium and/or periplasmic space of the host cell.

Accordingly, the present invention, also provides a method for producing a protein product of choice, the method comprising:

(a) providing a host cell of the invention comprising a polynucleotide encoding protein product of choice as defined above; and
(b) growing the host cell (for example, culturing the host cell in a culture medium);
thereby to produce a cell culture or recombinant organism comprising an increased level of the protein product of choice compared to the level of production of the protein product of choice achieved by growing (for example, culturing), under the same conditions, the same host cell that has not been genetically modified to cause over-expression of one or more helper proteins.

The step of growing the host cell may or may not involve allowing a host cell derived from a multicellular organism to be regrown into a multicellular recombinant organism (such as a plant or animal) and, optionally, producing one or more generations of progeny therefrom.

The method may or may not further comprise the step of purifying the thus expressed protein product of choice from the cultured host cell, recombinant organism or culture medium.

The step of “purifying the thus expressed protein product of choice from the cultured host cell, recombinant organism or culture medium” optionally comprises cell immobilisation, cell separation and/or cell breakage, but always comprises at least one other purification step different from the step or steps of cell immobilisation, separation and/or breakage.

Cell immobilisation techniques, such as encasing the cells using calcium alginate bead, are well known in the art. Similarly, cell separation techniques, such as centrifugation, filtration.(e.g. cross-flow filtration, expanded bed chromatography and the like) are well known in the art. Likewise, methods of cell breakage, including beadmilling, sonication, enzymatic exposure and the like are well known in the art.

The “at least one other purification step” may be any other step suitable for protein purification known in the art. For example purification techniques for the recovery of recombinantly expressed albumin have been disclosed in: WO 92/04367, removal of matrix-derived dye; EP 464 590, removal of yeast-derived colorants; EP 319 067, alkaline precipitation and subsequent application of the albumin to a lipophilic phase; and WO 96/37515, U.S. Pat. No. 5,728,553 and WO 00/44772, which describe complete purification processes; all of which are incorporated herein by reference.

Proteins other than albumin may be purified from the culture medium by any technique that has been found to be useful for purifying such proteins.

Suitable methods include ammonium sulphate or ethanol precipitation, acid or solvent extraction, anion or cation exchange chromatography, phosphocellulose chromatography, hydrophobic interaction chromatography, affinity chromatography, hydroxyapatite chromatography, lectin chromatography, concentration, dilution, pH adjustment, diafiltration, ultrafiltration, high performance liquid chromatography (“HPLC”), reverse phase HPLC, conductivity adjustment and the like.

In one embodiment, any one or more of the above mentioned techniques may or may not be used to further purifying the thus isolated protein to a commercially or industrially acceptable level of purity. By commercially or industrially acceptable level of purity, we include the provision of the protein at a concentration of at least 10⁴ g·L⁻¹, 10⁻³ g·L⁻¹, 0.01 g·L⁻¹, 0.02 g·L⁻¹, 0.03 g·L⁻¹, 0.04 g·L⁻¹, 0.05 g·L⁻¹, 0.06 g·L⁻¹, 0.07 g·L⁻¹, 0.08 g·L⁻¹, 0.09 g·L⁻¹, 0.1 g·L⁻¹, 0.2 g·L⁻¹, 0.3 g·L⁻¹, 0.4 g·L⁻¹, 0.5 g·L⁻¹, 0.6 g·L⁻¹, 0.7 g·L⁻¹, 0.8 g·L⁻¹, 0.9 g·L⁻¹, 1 g·L⁻¹, 2 g·L⁻¹, 3 g·L⁻¹, 4 g·L⁻¹, 5 g·L⁻¹, 6 g·L⁻¹, 7 g·L⁻¹, 8 g·L⁻¹, 9 g·L⁻¹, 10 g·L⁻¹, 15 g·L⁻¹, 20 g·L⁻¹, 25 g·L⁻¹, 30 g·L⁻¹, 40 g·L⁻¹, 50 g·L⁻¹, 60 g·L⁻¹, 70 g·L⁻¹, 70 g·L⁻¹, 90 g·L⁻¹, 100 g·L⁻¹, 150 g·L⁻¹, 200 g·L⁻¹, 250 g·L⁻¹, 300 g·L⁻¹, 350 g·L⁻¹, 400 g·L⁻¹, 500 g·L⁻¹, 600 g·L⁻¹, 700 g·L⁻¹, 800 g·L⁻¹, 900 g·L⁻¹, 1000 g·L⁻¹, or more.

A commercially or industrially acceptable level of purity may be obtained by a relatively crude purification method by which the protein product of choice is put into a form suitable for its intended purpose. A protein preparation that has been purified to a commercially or industrially acceptable level of purity may, in addition to the protein product of choice, also comprise, for example, cell culture components such as host cells or debris derived therefrom. Alternatively, high molecular weight components (such as host cells or debris derived therefrom) may or may not be removed (such as by filtration or centrifugation) to obtain a composition comprising the protein product of choice and, optionally, a functionally acceptable level of low molecular weight contaminants derived from the cell culture process.

The protein may or may not be purified to achieve a pharmaceutically acceptable level of purity. A protein has a pharmaceutically acceptable level of purity if it is essentially pyrogen free and can be administered in a pharmaceutically efficacious amount without causing medical effects not associated with the activity of the protein.

The resulting protein may be used for any of its known utilities, which, in the case of albumin, include i.v. administration to patients to treat severe burns, shock and blood loss, supplementing culture media, and as an excipient in formulations of other proteins.

A method of the present invention may or may not further comprise the step of formulating the purified protein product of choice with a carrier or diluent and optionally presenting the thus formulated protein in a unit dosage form.

Although it is possible for a therapeutically useful protein obtained by a process of the invention to be administered alone, it is preferable to present it as a pharmaceutical formulation, together with one or more acceptable carriers or diluents. The carrier(s) or diluent(s) must be “acceptable” in the sense of being compatible with the desired protein and not deleterious to the recipients thereof. Typically, the carriers or diluents will be water or saline which will be sterile and pyrogen free.

Optionally the thus formulated protein will be presented in a unit dosage form, such as in the form of a tablet, capsule, injectable solution or the like.

Alternatively, a method of the present invention may or may not further comprise the step of lyophilising the thus purified protein product of choice.

### Detailed Description of Helper Proteins

JEM1 is one S. cerevisiae helper protein of interest for the present invention. It is also known as KAR8, and its gene is a non-essential gene located on chromosome X. It is a DnaJ-like chaperone and is thought to be required for nuclear membrane fusion during mating. It localises to the ER membrane and exhibits genetic interactions with Kar2p (described further below). A published protein sequence for the protein Jem1p is as follows:

MILISGYCLLVYSVILPVLISASKLCDLAELQRLNKNLKVDTESLPKYQWIAGQLEQNCM
TADPASENMSDVIQLANQIYYKIGLIQLSNDQHLRAINTFEKIVFNETYKGSFGKLAEKR
LQELYVDFGMWDKVHQKDDQYAKYLSLNETIRNKISSKDVSVEEDISELLRITPYDVNVL
STHIDVLFHKLAEEIDVSLAAAIILDYETILDKHLASLSIDTRLSIHYVISVLQTFVLNS
DASFNIRKCLSIDMDYDKCKKLSLTISKLNKVNPSKRQILDPATYAFENKKFRSWDRIIE
FYLKDKKPFITPMKILNKDTNFKNNYFFLEEIIKQLIEDVQLSRPLAKNLFEDPPITDGF
VKPKSYYHTDYLVYIDSILCQASSMSPDVKRAKLAAPFCKKSLRHSLTLETWKHYQDAKS
EQKPLPETVLSDVWNSNPHLLMYMVNSILNKSRSKPHSQFKKQLYDQINKFFQDNGLSES
TNPYVMKNFRLLQKQLQTYKEHKHRNFNQQYFQQQQQQQQHQRHQAPPAAPNYDPKKDYY
KILGVSPSASSKEIRKAYLNLTKKYHPDKIKANHNDKQESIHETMSQINEAYETLSDDDK
RKEYDLSRSNPRRNTFPQGPRQNNMFKNPGSGFPFGNGFKMNFGL*

The ORF of the JEM1 gene is 1.938 kbp in size. A published nucleotide coding sequence of JEM1 is as follows, although it will be appreciated that the sequence on be modified by degenerate substitutions to obtain alternative nucleotide sequences which encode an identical protein product:

ATGATACTGATCTCGGGATACTGTCTTTTAGTGTATAGCGTTATTTTGCCAGTACTGATA
TCGGCTTCTAAGTTATGTGATTTGGCTGAGTTACAACGATTGAACAAGAATTTAAAAGTA
GACACTGAATCCTTGCCAAAATACCAATGGATCGCTGGGCAGTTGGAACAAAACTGCATG
ACTGCGGATCCAGCAAGTGAAAATATGTCAGACGTAATTCAACTAGCCAATCAAATATAC
TACAAAATTGGGCTGATCCAATTATCCAACGATCAACATCTAAGAGCTATTAACACATTT
GAAAAAATCGTTTTTAATGAAACTTACAAAGGTTCTTTTGGGAAGCTGGCGGAAAAGAGG
CTACAAGAGCTGTATGTCGATTTTGGGATGTGGGACAAGGTGCATCAGAAGGATGATCAG
TATGCGAAATATCTGTCCTTGAATGAAACCATCAGAAACAAAATATCATCCAAAGACGTT
TCTGTGGAGGAAGATATTTCTGAGCTGCTACGCATAACGCCGTACGATGTTAACGTCCTC
TCCACGCACATCGATGTTCTTTTTCACAAACTAGCTGAAGAAATTGACGTTTCGTTAGCT
GCTGCTATCATTTTGGATTACGAAACAATCCTCGACAAGCATTTGGCTAGCTTAAGCATA
GATACAAGACTTTCGATTCATTATGTCATATCTGTTTTACAGACCTTTGTACTTAACTCA
GATGCGTCGTTCAATATAAGAAAATGCCTTTCCATTGATATGGACTATGATAAATGTAAA
AAACTAAGCCTGACTATTTCCAAATTGAACAAGGTGAATCCATCAAAAAGACAGATCCTG
GATCCAGCAACATATGCATTTGAGAACAAAAAGTTTAGAAGTTGGGATAGAATTATTGAA
TTTTATTTGAAGGATAAGAAGCCATTTATTACACCAATGAAAATTCTTAACAAAGATACA
AACTTTAAAAACAACTACTTCTTTTTAGAGGAAATTATCAAACAATTGATAGAAGACGTT
CAACTGTCGAGACCTTTGGCAAAAAATTTATTCGAAGATCCCCCAATAACCGATGGTTTT
GTCAAACCAAAATCATACTATCATACCGATTATCTAGTATACATTGATTCCATTCTTTGT
CAGGCTTCTAGCATGAGTCCGGACGTCAAGAGAGCTAAACTGGCTGCGCCGTTCTGTAAA
AAGAGTTTGAGGCATTCACTAACACTAGAAACATGGAAACACTATCAGGATGCTAAGTCC
GAGCAAAAACCTTTACCTGAGACGGTATTGAGTGATGTATGGAATTCCAATCCTCATTTG
CTGATGTATATGGTAAACTCAATACTTAATAAAAGTAGGTCTAAACCTCATTCACAGTTC
AAAAAGCAATTATATGACCAGATAAACAAATTTTTCCAAGATAACGGCCTCTCAGAGTCG
ACCAATCCATACGTGATGAAGAACTTCCGATTATTACAGAAACAATTACAAACCTATAAA
GAGCATAAACATCGGAATTTCAACCAGCAATATTTCCAACAACAACAACAGCAGCAACAA
CACCAACGACATCAAGCACCCCCAGCAGCGCCTAACTACGACCCAAAAAAGGACTATTAT
AAAATTCTTGGAGTATCGCCTAGTGCTAGTTCGAAAGAAATAAGGAAAGCATATTTAAAT
TTAACCAAAAAATACCACCCAGACAAAATAAAGGCCAACCATAACGACAAACAAGAATCA
ATTCACGAAACTATGTCACAAATCAATGAAGCGTACGAAACATTAAGTGATGACGATAAA
AGGAAGGAATACGATCTTTCCAGATCAAACCCCCGCCGCAACACTTTTCCTCAGGGGCCT
AGGCAAAATAACATGTTCAAAAATCCAGGAAGTGGCTTCCCATTCGGAAATGGCTTTAAA
ATGAATTTTGGGCTTTGA

Further information concerning JEM1 can be seen at the following URL address http://db.yeastgenome.org/cgi-bin/singlepageformat?sgdid=S000003609.

It will be appreciated that, by “JEM1”, we include fragments or variants thereof having equivalent JEM1-like activity. Such variants may or may not include bacterial DnaJ proteins and/or may or may not include eukaryotic DnaJ type proteins, such as other members of the Hsp40 family. In one embodiment, a variant of JEM1 may not be SCJ1.

LHS1 is another S. cerevisiae helper protein of interest for the present invention. It is also known as CER1 or SSI1, is encoded by a non-essential gene which is located on chromosome XI. It is thought to be a molecular chaperone of the endoplasmic reticulum lumen, involved in polypeptide translocation and folding. It is a member of the HSP70 family, localizes to the lumen of the ER, and is thought to be regulated by the unfolded protein response pathway.

A published protein sequence for the protein Lhs1p is as follows:

MRNVLRLLFLTAFVAIGSLAAVLGVDYGQQNIKAIVVSPQAPLELVLTPEAKRKEISGLS
IKRLPGYGKDDPNGIERIYGSAVGSLATRFPQNTLLHLKPLLGKSLEDETTVTLYSKQHP
GLEMVSTNRSTIAFLVDNVEYPLEELVAMNVQEIANRANSLLKDRDARTEDFVNKMSFTI
PDFFDQHQRKALLDASSITTGIEETYLVSEGMSVAVNFVLKQRQFPPGEQQHYIVYDMGS
GSIKASMFSILQPEDTTQPVTIEFEGYGYNPHLGGAKFTMDIGSLIENKFLETHPAIRTD
ELHANPKALAKINQAAEKAKLILSANSEASINIESLINDIDFRTSITRQEFEEFIADSLL
DIVKPINDAVTKQFGGYGTNLPEINGVILAGGSSRIPIVQDQLIKLVSEEKVLRNVNADE
SAVNGVVMRGIKLSNSFKTKPLNVVDRSVNTYSFKLSNESELYDVFTRGSAYPNKTSILT
NTTDSIPNNFTIDLFENGKLFETITVNSGAIKNSYSSDKCSSGVAYNITFDLSSDRLFSI
QEVNCICQSENDIGNSKQIKNKGSRLAFTSEDVEIKRLSPSERSRLHEHIKLLDKQDKER
FQFQENLNVLESNLYDARNLLMDDEVMQNGPKSQVEELSEMVKVYLDWLEDASFDTDPED
IVSRIREIGILKKKIELYMDSAKEPLNSQQFKGMLEEGHKLLQAIETHKNTVEEFLSQFE
TEFADTIDNVREEFKKIKQPAYVSKALSTWEETLTSFKNSISEIEKFLAKNLFGEDLREH
LFEIKLQFDMYRTKLEEKLRLIKSGDESRLNEIKKLHLRNFRLQKRKEEKLKRKLEQEKS
RNNNETESTVINSADDKTTIVNDKTTESNPSSEEDILHDEL*

The ORF of the LHS1 gene is 2.646 kbp in size. A published nucleotide coding sequence of LHS1 is as follows, although it will be appreciated that the sequence can be modified by degenerate substitutions to obtain alternative nucleotide sequences which encode an identical protein product:

ATGCGAAACGTTTTAAGGCTTTTATTTTTAACAGCTTTTGTTGCTATAGGGTCTTTAGCA
GCCGTTTTAGGTGTTGATTACGGTCAGCAAAATATCAAGGCCATTGTGGTTTCTCCGCAA
GCCCCATTAGAACTTGTGCTCACACCAGAGGCAAAACGGAAGGAGATATCTGGTCTTTCG
ATAAAAAGATTACCAGGTTATGGAAAGGATGATCCGAATGGGATTGAAAGAATCTACGGT
TCCGCTGTTGGCAGTTTAGCAACAAGGTTTCCCCAAAACACATTGTTGCATTTGAAACCG
CTACTTGGGAAATCACTAGAAGATGAAACCACTGTAACTTTGTATTCAAAACAACACCCC
GGTTTAGAAATGGTATCAACAAATAGAAGTACCATAGCCTTTTTAGTTGATAATGTGGAA
TATCCATTGGAAGAGTTAGTGGCAATGAATGTCCAAGAGATTGCCAATAGAGCCAATTCA
CTGTTGAAGGATAGAGATGCAAGAACTGAGGACTTTGTAAACAAGATGAGTTTTACAATT
CCTGACTTTTTTGACCAACATCAAAGGAAAGCACTTTTAGATGCCAGTTCAATAACCACA
GGAATCGAAGAGACATATCTGGTTAGTGAAGGGATGTCTGTTGCAGTTAACTTTGTATTA
AAGCAGCGCCAATTTCCACCAGGTGAACAGCAGCATTATATCGTATATGACATGGGGAGC
GGTTCTATTAAGGCCTCAATGTTCTCTATATTGCAGCCGGAGGACACTACTCAGCCCGTT
ACAATAGAATTTGAAGGATATGGGTATAATCCACATCTAGGTGGTGCAAAGTTTACAATG
GATATTGGCAGTTTGATAGAGAATAAGTTTTTGGAAACACACCCAGCCATAAGAACTGAT
GAATTGCACGCTAATCCCAAGGCCTTAGCAAAAATCAACCAAGCAGCAGAGAAGGCAAAG
TTAATTTTAAGCGCCAATTCTGAGGCAAGTATTAACATAGAATCACTGATCAACGATATT
GATTTCCGTACTTCTATAACTAGACAGGAATTCGAAGAATTTATTGCAGACTCGTTATTG
GACATTGTCAAACCCATAAATGACGCTGTTACAAAACAATTCGGTGGCTATGGAACAAAT
TTACCTGAGATAAATGGGGTCATTTTGGCGGGAGGCTCTTCCCGAATTCCCATTGTGCAG
GATCAATTAATCAAACTCGTATCCGAAGAAAAAGTGTTGAGAAATGTCAATGCTGATGAA
TCAGCTGTGAATGGTGTTGTTATGAGAGGGATCAAGTTATCTAATTCGTTTAAGACCAAG
CCGTTAAATGTTGTTGACCGTTCTGTAAATACTTATTCATTCAAATTATCAAACGAATCT
GAACTGTATGATGTGTTCACGCGCGGAAGTGCTTATCCAAACAAAACATCTATTTTGACA
AACACGACTGATTCGATTCCTAATAATTTTACCATTGACTTATTTGAGAATGGTAAATTG
TTCGAAACTATCACAGTTAATTCAGGAGCTATAAAGAATTCATATTCCTCTGATAAGTGC
TCGTCAGGAGTTGCGTATAACATTACTTTCGACTTGTCCAGTGATAGATTATTCTCTATT
CAAGAGGTTAACTGCATTTGTCAGAGCGAAAATGACATAGGTAACTCCAAGCAAATTAAG
AACAAAGGCAGCCGTTTGGCTTTTACTTCTGAGGATGTTGAGATCAAAAGGCTTTCTCCT
TCAGAACGTTCGCGTTTGCATGAGCATATCAAGTTGCTCGATAAACAGGATAAGGAAAGA
TTTTCAATTCCAAGAAAATTTAAACGTTCTTGAAAGTAACTTGTATGATGCTAGAAACCTG
CTAATGGATGATGAAGTTATGCAAAATGGACCAAAATCCCAAGTAGAAGAGTTATCGGAG
ATGGTTAAAGTATATTTGGATTGGCTCGAAGATGCATCCTTTGATACTGACCCTGAGGAT
ATAGTTAGCAGAATTAGAGAAATTGGAATATTAAAAAAGAAAATAGAACTTTACATGGAT
TCTGCAAAGGAACCTTTGAACTCTCAACAATTTAAAGGAATGCTTGAAGAAGGCCATAAG
TTACTTCAGGCTATAGAAACCCATAAGAATACCGTTGAAGAATTTTTGAGTCAATTTGAA
ACCGAGTTTGCGGATACCATAGATAATGTTAGAGAAGAATTTAAAAAGATTAAGCAACCA
GCGTATGTGTCGAAGGCGTTATCTACATGGGAGGAAACCTTAACCTCTTTTAAAAATTCC
ATTAGCGAAATAGAGAAGTTCCTGGCAAAAAACCTATTTGGCGAAGACCTTCGTGAACAT
TTATTTGAAATCAAATTACAATTTGATATGTATCGTACGAAACTAGAGGAAAAACTGCGT
TTAATAAAAAGCGGTGATGAAAGTCGCTTAAATGAAATAAAGAAGTTACATTTAAGAAAC
TTCCGCCTACAAAAGAGAAAGGAGGAAAAGTTGAAAAGAAAGCTTGAACAGGAAAAAAGC
AGAAACAACAATGAAACAGAATCGACAGTAATCAACTCGGCTGACGATAAAACTACTATT
GTCAATGACAAGACCACCGAGTCGAATCCAAGTTCTGAGGAAGACATTTTGCATGATGAA
TTATAG

Further information on LHS1 can be obtained from the URL address http://db.yeastgenome.org/cgi-bin/singlepageformat?sgdid=S000001556.

It will be appreciated that, by “LHS1”, we include fragments or variants thereof having equivalent LHS1-like activity. Such variants may or may not include bacterial DnaK proteins and/or eukaryotic DnaK type proteins, such as other members of the Hsp70 family.

SCJ1 is another S. cerevisiae helper protein of interest for the present invention. It is one of several homologs of bacterial chaperone DnaJ, located in the ER lumen where it cooperates with Kar2p (described below) to mediate maturation of proteins.

A published protein sequence for the protein Scj1p is as follows:

MIPKLYIHLILSLLLLPLILAQDYYAILEIDKDATEKEIKSAYRQLSKKY
HPDKNAGSEEAHQKFIEVGEAYDVLSDPEKKKIYDQFGADAVKNGGGGGG
PGGPGAGGFHDPFDIFERMFQGGHGGPGGGFGQRQRQRGPMIKVQEKLSL
KQFYSGSSIEFTLNLNDECDACHGSGSADGKLAQCPDCQGRGVIIQVLRM
GIMTQQIQQMCGRCGGTGQIIKNECKTCHGKKVTKKNKFFHVDVPPGAPR
NYMDTRVGEAEKGPDFDAGDLVIEFKEKDTENMGYRRRGDNLYRTEVLSA
AEALYGGWQRTIEFLDENKPVKLSRPAHVVVSNGEVEVVKGFGMPKGSKG
YGDLYIDYVVVMPKTFKSGQNMLKDEL*

SCJ1 is encoded by a non-essential gene comprising an ORF of 1.134 kbp. The gene is located on chromosome XIII. A published nucleotide coding sequence of SCJ1 is as follows, although it will be appreciated that the sequence can be modified by degenerate substitutions to obtain alternative nucleotide sequences which encode an identical protein product:

ATGATTCCAAAATTATATATACATTTGATACTATCTTTATTGTTGTTGCCGCTAATTTTG
GCGCAGGATTATTATGCAATACTAGAGATAGACAAAGATGCCACTGAGAAGGAAATCAAA
TCAGCGTACAGACAATTGTCTAAGAAGTACCATCCGGATAAAAATGCTGGGAGCGAAGAA
GCCCATCAAAAATTCATTGAAGTCGGCGAGGCATACGATGTATTGAGCGATCCTGAAAAG
AAAAAGATTTATGACCAGTTTGGTGCAGATGCTGTAAAGAATGGCGGTGGCGGTGGCGGT
CCAGGAGGCCCTGGCGCAGGTGGATTCCACGATCCGTTTGACATATTCGAACGGATGTTT
CAAGGAGGTCATGGAGGTCCTGGCGGCGGATTTGGCCAGAGACAGAGGCAGCGTGGTCCA
ATGATCAAGGTCCAGGAAAAACTATCTTTAAAGCAGTTTTATTCCGGGTCCTCGATAGAA
TTTACTTTAAACCTAAACGATGAATGTGATGCATGCCATGGTAGTGGCTCTGCAGATGGT
AAGCTGGCCCAATGTCCCGATTGTCAAGGTCGTGGGGTTATAATACAAGTGCTGCGCATG
GGTATTATGACGCAGCAGATTCAACAGATGTGTGGTAGGTGTGGTGGTACGGGACAAATT
ATCAAAAATGAATGCAAAACATGTCACGGCAAAAAAGTTACCAAAAAGAACAAGTTCTTC
CACGTTGACGTTCCACCAGGCGCACCAAGAAACTACATGGACACAAGAGTCGGCGAGGCT
GAAAAAGGGCCTGACTTTGACGCCGGTGACTTGGTCATAGAATTCAAGGAAAAGGATACT
GAGAACATGGGTTACAGAAGAAGAGGCGACAATCTGTACAGAACAGAAGTTCTTTCTGCT
GCGGAAGCGCTATACGGCGGATGGCAAAGAACGATAGAATTCCTTGATGAGAACAAGCCC
GTTAAGTTATCTAGACCCGCTCATGTAGTTGTCTCCAATGGCGAAGTTGAAGTCGTGAAG
GGATTCGGCATGCCCAAGGGTAGCAAGGGTTACGGTGATTTGTACATAGACTACGTCGTT
GTCATGCCAAAGACTTTCAAATCTGGGCAAAATATGCTCAAAGATGAGTTGTAG

Further information on SCJ1 can be obtained from the URL address http://db.yeastgenome.org/cgi-bin/singlepageformat?sgdid=S006004827.

It will be appreciated that, by “SCJ1”, we include fragments or variants thereof having equivalent SCJ1-like activity.

KAR2 is another S. cerevisiae helper protein of interest for the present invention. KAR2 is also known as BIP or GRP78. Kar2p, is an ATPase involved in protein import into the ER. Kar2p also acts as a chaperone to mediate protein folding in the ER and may play a role in ER export of soluble proteins. It is also thought to regulate the unfolded protein response via interaction with kelp. A published protein sequence for the protein Kar2p is as follows:

MFFNRLSAGKLLVPLSVVLYALFVVILPLQNSFHSSNVLVRGADDVENYG
TVIGIDLGTTYSCVAVMKNGKTEILANEQGNRITPSYVAFTDDERLIGDA
AKNQVAANPQNTIFDIKRLIGLKYNDRSVQKDIKHLPFNVVNKDGKPAVE
VSVKGEKKVFTPEEISGMILGKMKQIAEDYLGTKVTHAVVTVPAYFNDAQ
RQATKDAGTIAGLNVLRIVNEPTAAAIAYGLDKSDKEHQIIVYDLGGGTF
DVSLLSIENGVFEVQATSGDTHLGGEDFDYKIVRQLIKAFKKKHGIDVSD
NNKALAKLKREAEKAKRALSSQMSTRIEIDSFVDGIDLSETLTRAKFEEL
NLDLFKKTLKPVEKVLQDSGLEKKDVDDIVLVGGSTRIPKVQQLLESYFD
GKKASKGINPDEAVAYGAAVQAGVLSGEEGVEDIVLLDVNALTLGIETTG
GVMTPLIKRNTAIPTKKSQIFSTAVDNQPTVMIKVYEGERAMSKDNNLLG
KFELTGIPPAPRGVPQIEVTFALDANGILKVSATDKGTGKSESITITNDK
GRLTQEEIDRMVEEAEKFASEDASIKAKVESRNKLENYAHSLKNQVNGDL
GEKLEEEDKETLLDAANDVLEWLDDNFETAIAEDFDEKFESLSKVAYPIT
SKLYGGADGSGAADYDDEDEDDDGDYFEHDEL*

KAR2 is encoded by an essential gene comprising an ORF that is 2.049 kbp in size and located on chromosome X. A published nucleotide coding sequence of KAR2 is as follows, although it will be appreciated that the sequence can be modified by degenerate substitutions to obtain alternative nucleotide sequences which encode an identical protein product:

ATGTTTTTCAACAGACTAAGCGCTGGCAAGCTGCTGGTACCACTCTCCGTGGTCCTGTAC
GCCCTTTTCGTGGTAATATTACCTTTACAGAATTCTTTCCACTCCTCCAATGTTTTAGTT
AGAGGTGCCGATGATGTAGAAAACTACGGAACTGTTATCGGTATTGACTTAGGTACTACT
TATTCCTGTGTTGCTGTGATGAAAAATGGTAAGACTGAAATTCTTGCTAATGAGCAAGGT
AACAGAATCACCCCATCTTACGTGGCATTCACCGATGATGAAAGATTGATTGGTGATGCT
GCAAAGAACCAAGTTGCTGCCAATCCTCAAAACACCATCTTCGACATTAAGAGATTGATC
GGTTTGAAATATAACGACAGATCTGTTCAGAAGGATATCAAGCACTTGCCATTTAATGTG
GTTAATAAAGATGGGAAGCCCGCTGTAGAAGTAAGTGTCAAAGGAGAAAAGAAGGTTTTT
ACTCCAGAAGAAATTTCTGGTATGATCTTGGGTAAGATGAAACAAATTGCCGAAGATTAT
TTAGGCACTAAGGTTACCCATGCTGTCGTTACTGTTCCTGCTTATTTCAATGACGCGCAA
AGACAAGCCACCAAGGATGCTGGTACCATCGCTGGTTTGAACGTTTTGAGAATTGTTAAT
GAACCAACCGCAGCCGCCATTGCCTACGGTTTGGATAAATCTGATAAGGAACATCAAATT
ATTGTTTATGATTTGGGTGGTGGTACTTTCGATGTCTCTCTATTGTCTATTGAAAACGGT
GTTTTCGAAGTCCAAGCCACTTCTGGTGATACTCATTTAGGTGGTGAAGATTTTGACTAT
AAGATCGTTCGTCAATTGATAAAAGCTTTCAAGAAGAAGCATGGTATTGATGTGTCTGAC
AACAACAAGGCCCTAGCTAAATTGAAGAGAGAAGCTGAAAAGGCTAAACGTGCCTTGTCC
AGCCAAATGTCCACCCGTATTGAAATTGACTCCTTCGTTGATGGTATCGACTTAAGTGAA
ACCTTGACCAGAGCTAAGTTTGAGGAATTAAACCTAGATCTATTCAAGAAGACCTTGAAG
CCTGTCGAGAAGGTTTTGCAAGATTCTGGTTTGGAAAAGAAGGATGTTGATGATATCGTT
TTGGTTGGTGGTTCTACTAGAATTCCAAAGGTCCAACAATTGTTAGAATCATACTTTGAT
GGTAAGAAGGCCTCCAAGGGTATTAACCCAGATGAAGCTGTTGCATACGGTGCAGCCGTT
CAAGCTGGTGTCTTATCCGGTGAAGAAGGTGTCGAAGATATTGTTTTATTGGATGTCAAC
GCTTTGACTCTTGGTATTGAAACCACTGGTGGTGTCATGACTCCATTAATTAAGAGAAAT
ACTGCTATTCCTACAAAGAAATCCCAAATTTTCTCTACTGCCGTTGACAACCAACCAACC
GTTATGATCAAGGTATACGAGGGTGAAAGAGCCATGTCTAAGGACAACAATCTATTAGGT
AAGTTTGAATTAACCGGCATTCCACCAGCACCAAGAGGTGTACCTCAAATTGAAGTCACA
TTTGCACTTGACGCTAATGGTATTCTGAAGGTGTCTGCCACAGATAAGGGAACTGGTAAA
TCCGAATCTATCACCATCACTAACGATAAAGGTAGATTAACCCAAGAAGAGATTGATAGA
ATGGTTGAAGAGGCTGAAAAATTCGCTTCTGAAGACGCTTCTATCAAGGCCAAGGTTGAA
TCTAGAAACAAATTAGAAAACTACGCTCACTCTTTGAAAAACCAAGTTAATGGTGACCTA
GGTGAAAAATTGGAAGAAGAAGACAAGGAAACCTTATTAGATGCTGCTAACGATGTTTTA
GAATGGTTAGATGATAACTTTGAAACCGCCATTGCTGAAGACTTTGATGAAAAGTTCGAA
TCTTTGTCCAAGGTCGCTTATCCAATTACTTCTAAGTTGTACGGAGGTGCTGATGGTTCT
GGTGCCGCTGATTATGACGACGAAGATGAAGATGACGATGGTGATTATTTCGAACACGAC
GAATTGTAG

Further information on KAR2 can be obtained from the URL address http://db.yeastgenome.org/cgi-bin/singlepageformat?sgdid=S000003571.

It will be appreciated that, by “KAR2”, we include fragments or variants thereof having equivalent KAR2-like activity.

SIL1 is another S. cerevisiae helper protein of interest for the present invention and is also known as SLS1. In particular, this helper protein was generally referred to as SLS1 in UK patent application no. 0512707.1, from which this application claims priority; it will be understood by the person skilled in the art that reference in UK patent application no. 0512707.1 to SLS1 and reference in this application to SIL1 should be taken to be reference to the same helper protein. SIL1p is an ER-localized protein required for protein translocation into the ER, which interacts with the ATPase domain of the Kar2p chaperone suggesting some role in modulating its activity. It is also thought to be a homolog of Yarrowia lipolytica SIL1 and a GrpE-like protein in the ER. A published protein sequence for the protein SIL1p is as follows:

MVRILPIILSALSSKLVASTILHSSIHSVPSGGEIISAEDLKELEISGNS
ICVDNRCYPKIFEPRHDWQPILPGQELPGGLDIRINMDTGLKEAKLNDEK
NVGDNGSHELIVSSEDMKASPGDYEFSSDFKEMRNIIDSNPTLSSQDIAR
LEDSFDRIMEFAHDYKHGYKIITHEFALLANLSLNENLPLTLRELSTRVI
TSCLRNNPPVVEFINESFPNFKSKIMAALSNLNDSNHRSSNILIKRYLSI
LNELPVTSEDLPIYSTVVLQNVYERNNKDKQLQIKVLELISKILKADMYE
NDDTNLILFKRNAENWSSNLQEWANEFQEMVQNKSIDELHTRTFFDTLYN
LKKIEFSDITINKGFLNWLAQQCKARQSNLDNGLQERDTEQDSFDKKLID
SRHLIFGNPMAHRIKNFRDEL*

SIL1 is encoded by a non-essential gene comprising an ORF that is 1.226 kbp in size and is located on chromosome XV. A published nucleotide coding sequence of SIL1 is as follows, although it will be appreciated that the sequence can be modified by degenerate substitutions to obtain alternative nucleotide sequences which encode an identical protein product:

ATGGTCCGGATTCTTCCCATAATTTTGAGCGCCCTATCTTCGAAATTAGTGGCGAGTACA
ATATTGCATTCATCCATACACTCAGTGCCATCTGGAGGCGAAATCATATCTGCAGAAGAT
CTTAAAGAACTTGAAATTTCAGGGAATTCGATCTGCGTTGATAATCGTTGCTATCCTAAG
ATATTTGAACCAAGACACGATTGGCAGCCCATACTGCCAGGTCAAGAACTCCCCGGTGGT
TTGGACATTAGAATAAACATGGACACAGGTTTAAAAGAGGCAAAACTAAATGATGAGAAG
AATGTCGGTGATAATGGTAGCCATGAGTTAATTGTATCTTCAGAAGACATGAAAGCATCG
CCTGGTGACTATGAATTTTCCAGTGATTTCAAAGAAATGAGAAACATCATAGATTCTAAC
CCGACTTTATCTTCACAGGACATTGCCAGATTGGAGGATAGTTTTGATAGAATAATGGAA
TTTGCGCATGATTACAAGCACGGCTACAAAATTATTACCCATGAATTCGCCCTCTTGGCC
AACCTTAGTCTCAATGAAAATTTGCCGTTAACATTGAGAGAGCTCAGTACTAGAGTCATT
ACCAGCTGCTTGAGAAACAATCCTCCTGTAGTCGAGTTCATTAATGAAAGTTTTCCAAAT
TTTAAAAGCAAAATCATGGCCGCTCTGTCAAATTTGAATGATTCTAACCACAGATCCTCT
AATATCCTAATAAAAAGATACTTGTCCATTTTAAACGAATTACCTGTCACATCCGAAGAT
CTTCCTATATACTCTACGGTTGTTTTACAAAATGTATATGAAAGAAACAACAAGGACAAA
CAGTTACAAATAAAAGTCCTGGAGTTGATCAGCAAAATTTTGAAGGCCGACATGTACGAA
AATGACGATACAAATCTAATTTTGTTCAAAAGAAATGCTGAGAATTGGTCGTCAAATCTG
CAAGAGTGGGCAAACGAGTTCCAAGAGATGGTCCAGAACAAAAGTATAGATGAACTACAT
ACAAGAACGTTTTTTGACACCCTTTACAACTTGAAGAAAATTTTCAAAAGTGACATCACG
ATCAACAAAGGGTTTTTGAATTGGTTAGCGCAACAATGTAAAGCCAGGCAATCTAACTTG
GACAATGGGCTCCAAGAGAGAGATACTGAACAAGACTCATTTGATAAGAAACTTATCGAC
AGCAGACACTTGATCTTTGGCAACCCCATGGCTCATAGAATAAAAAATTTCAGAGATGAA
CTCTGA

Further information on SIL1 can be obtained from the URL address http://db.yeastgenome.org/cgi-bin/singlepageformat?sgdid=S000005391.

It will be appreciated that, by “SIL1”, we include fragments or variants (including homologues) thereof having equivalent SIL1-like activity. In one embodiment, variants of SIL1 may or may not include bacterial GrpE type proteins and/or animal (such as mammalian) GrpE-like proteins. Variants of SIL1 may be a nucleotide exchange factor for an Hsp70 family protein, which nucleotide exchange factor is optionally not an Hsp70 family protein in itself. Suitable variants of SIL1 may or may not be FES1 and/or MGE1. A variant of SIL1 may or may not be localised to the lumen of the ER (such as SIL1 itself) to the mitochondria (such as MGE1) or to the cytosol (such as FES1). A variant of SIL1 may or may not include proteins such as members so of the mammalian GrpE-like protein family, the NEF family or BAG-1 (such as described in Hohfeld and Jentsch (1997) EMBO J. 16, 6209), mammalian BiP-associated protein (BAP) (Chung et al (2002) J. Biol. Chem. 277, 47557), a human GrpE-like protein (e.g. the protein defined by accession number AAG31605) (Choglay et al (2001) Gene 267, 125), an Arabidopsis thaliana GrpE-like protein (for example, accession numbers AAK68792 and BAB08589) (Sato et al (1998) DNA Res. 5, 41), a Chlamydia trachomatis Protein grpE (HSP-70 cofactor) (e.g. accession number P36424), a Pongo pygmaeus adenine nucleotide exchange factor (e.g. accession number CAH89792), a Mus musculus mitochondrial GrpE-like 2 protein (e.g. accession number NP_{—}067271), a Mus musculus mitochondrial GrpE-like 1 protein (e.g. accession number NP_{—}077798), a Gallus gallus GrpE protein homolog 2, mitochondrial precursor (Mt-GrpE#2) (e.g. accession number XP_{—}425191), a Gallus gallus BiP-associated protein (e.g. accession number XP_{—}414514), an Haemophilus influenzae 86-028NP GrpE protein (e.g. as defined by accession number YP_{—}247735) (Harrison et al (2005) J. Bacteriol. 187, 4627), an Escherichia coli GrpE heat shock protein (e.g. as defined by accession number NP_{—}417104) (Riley et al (1997) Science 277, 1453), a Streptococcus pneumoniae GrpE heat shock protein (e.g. as defined by accession number AAD23453), a Bacillus subtilis GrpE protein accession number (e.g. as defined by BAA12463) (Mizuno et al (1996) Microbiology (Reading, Engl.) 142, 3103) and/or a Nicotiana tabacum chaperone GrpE type 1 or GrpE type 2 protein (e.g. as defined by accession numbers AAC72386 or AAC72387) (Padidam et al (1999) Plant Mol. Biol. 39, 871).

Variants of SIL1 may have an activity equivalent to SIL1, when co-expressed with one or both of JEM1 and LHS1, for example in the manner as set out in the present examples. Thus, a host cell of the present invention, when genetically modified to cause simultaneous over-expression of a variant of SILT with one or both of JEM1 and LHS1, will provide at least substantially the same increase in the production of a protein product and/or at least substantially the same reduction of fragmentation of a protein product, as is observed in the same host cell when genetically modified to cause simultaneous over-expression of SIL1 with one or both of JEM1 and LHS1, the increase being compared to the level of production of the same protein product, and/or the level of fragmentation of the same protein product, in the same host cell that has not been genetically modified to cause overexpression of any of LHS1, JEM1 or SIL1.

By “substantially the same increase in the production of a protein product”, we mean at least 10%, 20%, 30%, 40%, 50%, 60%, 70%, 80%, 90%, 95%, 96%, 97%, 98%, 99%, substantially 100% or greater than 100% of the increase in production of a protein product that is observed when the host cell is genetically modified to cause simultaneous over-expression of SIL1 with one or both of JEM1 and LHS1 (the increased being compared to the level of production of the same protein product in the same host cell that has not been genetically modified to cause overexpression of any of LHS1, JEM1 or SIL1).

By “substantially the same reduction of fragmentation of a protein product”, we mean at least 10%, 20%, 30%, 40%, 50%, 60%, 70%, 80%, 90%, 95%, 96%, 97%, 98%, 99%, substantially 100% or greater than 100% of the reduction of fragmentation of a protein product that is observed when the host cell is genetically modified to cause simultaneous over-expression of SIL1 with one or both of JEM1 and LHS1 (the reduction of fragmentation of a protein product being compared to the level of fragmentation of the same protein product in the same host cell that has not been genetically modified to cause overexpression of any of LHS1, JEM1 or SIL1).

FKB2 is another S. cerevisiae helper protein of interest for the present invention and is also known as FPR2 and FKBP13. Fkb2p is a membrane bound peptidyl-prolyl cis-trans isomerase (PPIase) that binds to the drugs FK506 and rapamycin. The expression pattern of Fkb2p suggests possible involvement in ER protein trafficking. A published protein sequence for the protein Fkb2p is as follows:

MMFNIYLFVTFFSTILAGSLSDLEIGIIKRIPVEDCLIKAMPGDKVKVHY
TGSLLESGTVFDSSYSRGSPIAFELGVGRVIKGWDQGVAGMCVGEKRKLQ
IPSSLAYGERGVPGVIPPSADLVFDVELVDVKSAA*

FKB2 is encoded by a non-essential gene comprising an ORF that is 0.408 kbp in size and is located on chromosome IV. A published nucleotide coding sequence of FKB2 is as follows, although it will be appreciated that the sequence can be modified by degenerate substitutions to obtain alternative nucleotide sequences which encode an identical protein product:

ATGATGTTTAATATTTACCTTTTCGTCACTTTTTTTTCCACCATTCTTGC
AGGTTCCCTGTCAGATTTGGAAATCGGTATTATCAAGAGAATACCGGTAG
AAGATTGCTTAATTAAGGCAATGCCAGGTGATAAAGTTAAGGTTCATTAT
ACAGGATCTTTATTAGAATCGGGAACTGTATTTGACTCAAGTTATTCAAG
AGGCTCTCCTATCGCTTTTGAACTTGGCGTTGGCAGAGTAATTAAAGGTT
GGGATCAAGGTGTTGCCGGCATGTGCGTTGGCGAAAAAAGAAAGCTGCAA
ATTCCAAGTTCTTTGGCCTACGGAGAAAGAGGTGTCCCAGGCGTCATTCC
TCCAAGTGCTGATTTGGTGTTTGATGTCGAATTGGTAGACGTGAAATCAG
CCGCCTAG

Further information on FKB2 can be obtained from the URL address http://db.yeastgenome.org/cgi-bin/singlepageformat?sgdid=S000002927.

It will be appreciated that, by “FKB2”, we include fragments or variants thereof having equivalent FKB2-like activity.

SSA1 is another S. cerevisiae helper protein of interest for the present invention and is also known as YG100. Ssa1p is an ATPase that is involved in protein folding and nuclear localization signal (NLS)-directed nuclear transport. It is a member of heat shock protein 70 (HSP70) family. It forms a chaperone complex with Ydj1p and is localized to the nucleus, cytoplasm, and cell wall A published protein sequence for the protein Ssa1p is as follows:

MSKAVGIDLGTTYSCVAHFANDRVDIIANDQGNRTTPSFVAFTDTERLIG
DAAKNQAAMNPSNTVFDAKRLIGRNFNDPEVQADMKHFPFKLIDVDGKPQ
IQVEFKGETKNFTPEQISSMVLGKMKETAESYLGAKVNDAVVTVPAYFND
SQRQATKDAGTIAGLNVLRIINEPTAAAIAYGLDKKGKEEHVLIFDLGGG
TFDVSLLFIEDGIFEVKATAGDTHLGGEDFDNRLVNHFIQEFKRKNKKDL
STNQRALRRLRTACERAKRTLSSSAQTSVEIDSLFEGIDFYTSITRARFE
ELCADLERSTLDPVEKVLRDAKLDKSQVDEIVLVGGSTRIPKVQKLVTDY
FNGKEPNRSINPDEAVAYGAAVQAAILTGDESSKTQDLLLLDVAPLSLGI
ETAGGVMTKLIPRNSTISTKKFEIFSTYADNQPGVLIQVFEGERAKTKDN
NLLGKFELSGIPPAPRGVPQIEVTEDVDSNGILNVSAVEKGTGKSNKITI
TNDKGRLSKEDIEKMVAEAEKFKEEDEKESQRIASKNQLESIAYSLKNTI
SEAGDKLEQADKDTVTKKAEETISWLDSNTTASKEEFDDKLKELQDIANP
IMSKLYQAGGAPGGAAGGAPGGFPGGAPPAPEAEGPTVEEVD*

SSA1 is encoded by a non-essential gene comprising an ORF that is 1.929 kbp in size and is located on chromosome I. A published nucleotide coding sequence of SSA1 is as follows, although it will be appreciated that the sequence can be modified by degenerate substitutions to obtain alternative nucleotide sequences which encode an identical protein product:

ATGTCAAAAGCTGTCGGTATTGATTTAGGTACAACATACTCGTGTGTTGCTCACTTTGCT
AATGATCGTGTGGACATTATTGCCAACGATCAAGGTAACAGAACCACTCCATCTTTTGTC
GCTTTCACTGACACTGAAAGATTGATTGGTGATGCTGCTAAGAATCAAGCTGCTATGAAT
CCTTCGAATACCGTTTTCGACGCTAAGCGTTTGATCGGTAGAAACTTCAACGACCCAGAA
GTGCAGGCTGACATGAAGCACTTCCCATTCAAGTTGATCGATGTTGACGGTAAGCCTCAA
ATTCAAGTTGAATTTAAGGGTGAAACCAAGAACTTTACCCCAGAACAAATCTCCTCCATG
GTCTTGGGTAAGATGAAGGAAACTGCCGAATCTTACTTGGGAGCCAAGGTCAATGACGCT
GTCGTCACTGTCCCAGCTTACTTCAACGATTCTCAAAGACAAGCTACCAAGGATGCTGGT
ACCATTGCTGGTTTGAATGTCTTGCGTATTATTAACGAACCTACCGCCGCTGCCATTGCT
TACGGTTTGGACAAGAAGGGTAAGGAAGAACACGTCTTGATTTTCGACTTGGGTGGTGGT
ACTTTCGATGTCTCTTTGTTGTTCATTGAAGACGGTATCTTTGAAGTTAAGGCCACCGCT
GGTGACACCCATTTGGGTGGTGAAGATTTTGACAACAGATTGGTCAACCACTTCATCCAA
GAATTCAAGAGAAAGAACAAGAAGGACTTGTCTACCAACCAAAGAGCTTTGAGAAGATTA
AGAACCGCTTGTGAAAGAGCCAAGAGAACTTTGTCTTCCTCCGCTCAAACTTCCGTTGAA
ATTGACTCTTTGTTCGAAGGTATCGATTTCTACACTTCCATCACCAGAGCCAGATTCGAA
GAATTGTGTGCTGACTTGTTCAGATCTACTTTGGACCCAGTTGAAAAGGTCTTGAGAGAT
GCTAAATTGGACAAATCTCAAGTCGATGAAATTGTCTTGGTCGGTGGTTCTACCAGAATT
CCAAAGGTCCAAAAATTGGTCACTGACTACTTCAACGGTAAGGAACCAAACAGATCTATC
AACCCAGATGAAGCTGTTGCTTACGGTGCTGCTGTTCAAGCTGCTATTTTGACTGGTGAC
GAATCTTCCAAGACTCAAGATCTATTGTTGTTGGATGTCGCTCCATTATCCTTGGGTATT
GAAACTGCTGGTGGTGTCATGACCAAGTTGATTCCAAGAAACTCTACCATTTCAACAAAG
AAGTTCGAGATCTTTTCCACTTATGCTGATAACCAACCAGGTGTCTTGATTCAAGTCTTT
GAAGGTGAAAGAGCCAAGACTAAGGACAACAACTTGTTGGGTAAGTTCGAATTGAGTGGT
ATTCCACCAGCTCCAAGAGGTGTCCCACAAATTGAAGTCACTTTCGATGTCGACTCTAAC
GGTATTTTGAATGTTTCCGCCGTCGAAAAGGGTACTGGTAAGTCTAACAAGATCACTATT
ACCAACGACAAGGGTAGATTGTCCAAGGAAGATATCGAAAAGATGGTTGCTGAAGCCGAA
AAATTCAAGGAAGAAGATGAAAAGGAATCTCAAAGAATTGCTTCCAAGAACCAATTGGAA
TCCATTGCTTACTCTTTGAAGAACACCATTTCTGAAGCTGGTGACAAATTGGAACAAGCT
GACAAGGACACCGTCACCAAGAAGGCTGAAGAGACTATTTCTTGGTTAGACAGCAACACC
ACTGCCAGCAAGGAAGAATTCGATGACAAGTTGAAGGAGTTGCAAGACATTGCCAACCCA
ATCATGTCTAAGTTGTACCAAGCTGGTGGTGCTCCAGGTGGCGCTGCAGGTGGTGCTCCA
GGCGGTTTCCCAGGTGGTGCTCCTCCAGCTCCAGAGGCTGAAGGTCCAACCGTTGAAGAA
GTTGATTAA

Further information on SSA1 can be obtained from the URL address http://db.yeastgenome.org/cgi-bin/singlepageformat?sgdid=S000000004.

It will be appreciated that, by “SSA1”, we include fragments or variants thereof having equivalent SSA1-like activity.

SSA2 is another S. cerevisiae helper protein of interest for the present invention. Ssa2p is an ATP binding protein that is involved in protein folding and vacuolar import of proteins; member of heat shock protein 70 (HSP70) family. It is associated with the chaperonin-containing T-complex. It is present in the cytoplasm, vacuolar membrane and cell wall. A published protein sequence for the protein Ssa2p is as follows:

MSKAVGIDLGTTYSCVAHFSNDRVDIIANDQGNRTTPSFVGFTDTERLIG
DAAKNQAAMNPANTVFDAKRLIGRNFNDPEVQGDMKHFPFKLIDVDGKPQ
IQVEFKGETKNFTPEQISSMVLGKMKETAESYLGAKVNDAVVTVPAYFND
SQRQATKDAGTIAGLNVLRIINEPTAAAIAYGLDKKGKEEHVLIFDLGGG
TFDVSLLSIEDGIFEVKATAGDTHLGGEDFDNRLVNHFIQEFKRKNKKDL
STNQRALRRLRTACERAKRTLSSSAQTSVEIDSLFEGIDFYTSITRARFE
ELCADLFRSTLDPVEKVLRDAKLDKSQVDEIVLVGGSTRIPKVQKLVTDY
FNGKEPNRSINPDEAVAYGAAVQAAILTGDESSKTQDLLLLDVAPLSLGI
ETAGGVMTKLIPRNSTIPTKKSEVFSTYADNQPGVLIQVFEGERAKTKDN
NLLGKFELSGIPPAPRGVPQIEVTFDVDSNGILNVSAVEKGTGKSNKITI
TNDKGRLSKEDIEKMVAEAEKFKEEDEKESQRIASKNQLESIAYSLKNTI
SEAGDKLEQADKDAVTKKAEETIAWLDSNTTATKEEFDDQLKELQEVANP
IMSKLYQAGGAPEGAAPGGFPGGAPPAPEAEGPTVEEVD*

SSA2 is encoded by a non-essential gene comprising an ORF that is 1.920 kbp in size and is located on chromosome XII. A published nucleotide coding sequence of SSA2 is as follows, although it will be appreciated that the sequence can be modified by degenerate substitutions to obtain alternative nucleotide sequences which encode an identical protein product:

ATGTCTAAAGCTGTCGGTATTGATTTAGGTACTACCTACTCCTGTGTTGCTCACTTCTCT
AATGATCGTGTTGACATTATTGCCAACGACCAAGGTAACAGAACCACTCCATCTTTCGTT
GGTTTCACTGATACTGAAAGATTGATTGGTGACGCTGCTAAGAACCAAGCTGCTATGAAC
CCAGCTAACACTGTTTTCGACGCTAAGCGTTTGATCGGTAGAAACTTCAATGACCCAGAA
GTCCAAGGTGATATGAAGCACTTCCCATTCAAGTTGATCGATGTTGACGGTAAGCCACAA
ATTCAAGTTGAATTTAAGGGTGAAACCAAGAACTTTACCCCAGAACAAATCTCCTCCATG
GTCTTGGGTAAGATGAAGGAAACTGCCGAATCTTACTTGGGTGCCAAGGTCAATGACGCT
GTCGTCACTGTCCCAGCTTACTTCAACGATTCTCAAAGACAAGCTACCAAGGATGCTGGT
ACCATTGCTGGTTTGAATGTCTTGCGTATTATTAACGAACCTACCGCCGCTGCCATTGCT
TACGGTTTGGACAAGAAGGGTAAGGAAGAACACGTCTTGATTTTCGACTTGGGTGGTGGT
ACTTTCGATGTCTCTTTGTTGTCCATTGAAGACGGTATCTTTGAAGTTAAGGCCACCGCT
GGTGACACCCATTTGGGTGGTGAAGATTTTGACAACAGATTGGTCAACCACTTCATCCAA
GAATTCAAGAGAAAGAACAAGAAGGACTTGTCTACCAACCAAAGAGCTTTGAGAAGATTA
AGAACTGCTTGTGAAAGAGCCAAGAGAACTTTGTCTTCCTCCGCTCAAACTTCCGTTGAA
ATTGACTCTTTGTTCGAAGGTATCGATTTCTACACTTCCATCACCAGAGCCAGATTCGAA
GAATTGTGTGCTGACTTGTTCAGATCTACTTTGGACCCAGTTGAAAAGGTCTTGAGAGAT
GCTAAATTGGATAAATCTCAAGTCGATGAAATTGTCTTGGTCGGTGGTTCTACCAGAATT
CCAAAGGTCCAAAAATTGGTCACTGACTACTTCAACGGTAAGGAACCAAACAGATCTATC
AACCCAGATGAAGCTGTTGCTTACGGTGCTGCTGTTCAAGCTGCTATTTTGACTGGTGAC
GAATCTTCCAAGACTCAAGATCTATTGTTGTTGGATGTCGCTCCATTATCCTTGGGTATT
GAAACTGCTGGTGGTGTCATGACCAAGTTGATTCCAAGAAACTCTACCATTCCAACTAAG
AAATCCGAAGTTTTCTCTACTTATGCTGACAACCAACCAGGTGTCTTGATTCAAGTCTTT
GAAGGTGAAAGAGCCAAGACTAAGGACAACAACTTGTTGGGTAAGTTCGAATTGAGTGGT
ATTCCACCAGCTCCAAGAGGTGTCCCACAAATTGAAGTCACTTTCGATGTCGACTCTAAC
GGTATTTTGAATGTTTCCGCCGTCGAAAAGGGTACTGGTAAGTCTAACAAGATCACTATT
ACCAACGACAAGGGTAGATTGTCCAAGGAAGATATCGAAAAGATGGTTGCTGAAGCCGAA
AAATTCAAGGAAGAAGATGAAAAGGAATCTCAAAGAATTGCTTCCAAGAACCAATTGGAA
TCCATTGCTTACTCTTTGAAGAACACCATTTCTGAAGCTGGTGACAAGCTAGAGCAAGCT
GACAAGGACGCTGTCACTAAGAAGGCTGAAGAAACTATTGCTTGGTTAGACAGCAACACC
ACTGCTACCAAGGAAGAATTCGATGACCAATTGAAGGAATTGCAAGAGGTTGCCAACCCA
ATCATGTCTAAATTGTACCAAGCTGGTGGTGCTCCAGAAGGCGCAGCTCCAGGTGGTTTC
CCAGGTGGTGCTCCTCCAGCTCCAGAAGCTGAAGGTCCAACTGTCGAAGAAGTTGATTAA

Further information on SSA2 can be obtained from the URL address http://db.yeastgenome.org/cgi-bin/singlepageformat?sgdid=S000003947.

It will be appreciated that, by “SSA2”, we include fragments or variants thereof having equivalent SSA2-like activity.

SSA3 is another S. cerevisiae helper protein of interest for the present invention, which is also known as HSP70. Ssa3p is an ATPase involved in protein folding and the response to stress. It plays a role in SRP-dependent cotranslational protein-membrane targeting and translocation and is a member of the heat shock protein 70 (HSP70) family. SSA3 is localized to the cytoplasm. A published protein sequence for the protein Ssa3p is as follows:

MSRAVGIDLGTTYSCVAHFSNDRVEIIANDQGNRTTPSYVAFTDTERLIG
DAAKNQAAINPHNTVFDAKRLIGRKFDDPEVTTDAKHFPFKVISRDGKPV
VQVEYKGETKTFTPEEISSMVLSKMKETAENYLGTTVNDAVVTVPAYFND
SQRQATKDAGTIAGMNVLRIINEPTAAAIAYGLDKKGRAEHNVLIFDLGG
GTFDVSLLSIDEGVFEVKATAGDTHLGGEDFDNRLVNHLATEFKRKTKKD
ISNNQRSLRRLRTAAERAKRALSSSSQTSIEIDSLFEGMDFYTSLTRARF
EELCADLFRSTLEPVEKVLKDSKLDKSQIDEIVLVGGSTRIPKIQKLVSD
FFNGKEPNRSINPDEAVAYGAAVQAAILTGDQSTKTQDLLLLDVAPLSLG
IETAGGIMTKLIPRNSTIPTKKSETFSTYADNQPGVLIQVFEGERTRTKD
NNLLGKFELSGIPPAPRGVPQIDVTFDIDANGILNVSALEKGTGKSNKIT
ITNDKGRLSKDDIDRMVSEAEKYRADDEREAERVQAKNQLESYAFTLKNT
INEASFKEKVGEDDAKRLETASQETIDWLDASQAASTDEYKDRQKELEGI
ANPIMTKFYGAGAGAGPGAGESGGFPGSMPNSGATGGGEDTGPTVEEVD*

SSA3 is encoded by a non-essential gene comprising an ORF that is 1.950 kbp in size and is located on chromosome II. A published nucleotide coding sequence of SSA3 is as follows, although it will be appreciated that the sequence can be modified by degenerate substitutions to obtain alternative nucleotide sequences which encode an identical protein product:

ATGTCTAGAGCAGTTGGTATTGATTTGGGAACAACTTACTCGTGTGTTGCTCATTTTTCC
AATGATAGGGTAGAGATAATTGCAAATGATCAAGGTAATAGGACCACTCCATCGTATGTG
GCTTTCACAGACACCGAAAGATTAATTGGTGACGCCGCCAAAAATCAAGCTGCAATCAAT
CCTCATAATACAGTTTTTGATGCAAAGCGGTTAATTGGTCGTAAATTTGATGATCCTGAA
GTGACGACAGATGCCAAGCACTTCCCTTTCAAAGTTATATCCAGAGATGGTAAACCTGTA
GTGCAAGTAGAATATAAGGGTGAAACGAAAACATTTACGCCTGAGGAAATTTCTTCCATG
GTTTTAAGCAAAATGAAGGAAACTGCTGAGAACTATTTGGGAACTACGGTCAATGATGCT
GTTGTAACTGTTCCTGCATATTTCAATGATTCTCAAAGACAAGCCACTAAGGATGCAGGA
ACTATTGCAGGGATGAACGTTTTACGTATTATCAATGAACCCACTGCAGCAGCAATTGCT
TATGGCTTGGATAAGAAAGGCAGGGCTGAGCACAATGTCCTGATTTTTGATTTGGGTGGT
GGTACTTTTGACGTCTCTTTACTTTCAATTGATGAGGGTGTTTTTGAGGTTAAGGCTACC
GCAGGAGACACTCATTTAGGTGGTGAAGATTTTGATAATAGGTTGGTGAACCATTTAGCC
ACTGAATTCAAAAGGAAAACGAAAAAGGACATCTCTAATAATCAAAGATCGTTAAGAAGA
TTGAGAACTGCGGCAGAAAGAGCTAAGAGAGCGCTTTCTTCCTCATCTCAAACCTCGATC
GAGATCGATTCTTTATTTGAAGGTATGGATTTCTACACTTCGTTAACAAGGGCAAGGTTT
GAAGAGCTATGTGCTGATTTATTCAGATCCACATTGGAACCAGTAGAAAAGGTTCTTAAA
GATTCGAAGCTGGACAAGTCCCAAATTGATGAGATTGTGTTAGTCGGTGGATCTACCAGA
ATCCCAAAGATTCAGAAATTAGTTTCTGACTTCTTCAATGGCAAAGAGCCTAATCGTTCT
ATCAACCCGGATGAGGCTGTTGCTTATGGTGCAGCCGTTCAAGCTGCCATTTTAACCGGC
GATCAATCAACAAAGACACAAGATTTACTATTATTGGATGTTGCGCCATTGTCCCTAGGA
ATTGAAACTGCAGGCGGCATAATGACTAAGCTAATTCCTAGAAACTCAACGATTCCAACA
AAGAAATCGGAAACCTTCTCTACCTATGCAGATAATCAACCTGGTGTTTTAATTCAAGTC
TTTGAAGGTGAAAGAACAAGAACAAAGGATAATAACTTACTTGGTAAATTCGAATTAAGT
GGCATTCCGCCTGCTCCCAGAGGTGTGCCTCAAATTGATGTTACCTTTGATATCGACGCT
AATGGTATTCTTAATGTGTCTGCTTTGGAAAAGGGTACTGGTAAGAGTAACAAAATCACG
ATCACTAACGATAAAGGTAGGCTCTCGAAGGATGATATTGATAGGATGGTTTCTGAAGCT
GAAAAATATAGGGCTGACGATGAAAGGGAGGCAGAACGAGTTCAGGCTAAGAATCAGCTT
GAATCGTATGCATTTACTTTGAAGAATACCATAAACGAAGCAAGTTTCAAAGAGAAAGTA
GGTGAAGATGATGCAAAGAGATTAGAAACAGCGTCTCAGGAAACCATTGACTGGTTAGAT
GCATCGCAGGCAGCCTCTACGGACGAATATAAGGATAGACAAAAGGAGTTGGAAGGCATT
GCCAATCCAATAATGACGAAATTTTACGGTGCTGGTGCCGGCGCAGGTCCTGGAGCGGGG
GAATCCGGTGGATTCCCCGGATCCATGCCCAACTCGGGTGCTACGGGAGGTGGAGAAGAT
ACAGGTCCAACAGTGGAAGAGGTTGATTGA

Further information on SSA3 can be obtained from the URL address http://db.yeastgenome.org/cgi-bin/singlepageformat?sgdid=S000000171.

It will be appreciated that, by “SSA3”, we include fragments or variants thereof having equivalent SSA3-like activity.

SSA4 is another S. cerevisiae helper protein of interest for the present invention. Ssa4p is a heat shock protein that is highly induced upon stress. It plays a role in SRP-dependent cotranslational protein-membrane targeting and translocation; member of the HSP70 family. It is a cytoplasmic protein that concentrates in nuclei upon starvation. A published protein sequence for the protein Ssa4p is as follows:

MSKAVGIDLGTTYSCVAHFANDRVEIIANDQGNRTTPSYVAFTDTERLIG
DAAKNQAAMNPHNTVFDAKRLIGRKFDDPEVTNDAKHYPFKVIDKGGKPV
VQVEYKGETKTFTPEEISSMILTKMKETAENFLGTEVKDAVVTVPAYFND
SQRQATKDAGTIAGLNVLRIINEPTAAAIAYGLDKKSQKEHNVLIFDLGG
GTFDVSLLSIDEGVFEVKATAGDTHLGGEDFDSRLVNFLAEEFKRKNKKD
LTTNQRSLRRLRTAAERAKRTLSSSAQTSIEIDSLFEGIDFYTSITRARF
EELCADLFRSTLEPVEKVLADSKLDKSQIDEIVLVGGSTRIPKVQKLVSD
FFNGKEPNRSINPDEAVAYGAAVQAAILTGDQSSTTQDLLLLDVAPLSLG
IETAGGIMTKLIPRNSTIPTKKSEVFSTYADNQPGVLIQVFEGERTRTKD
NNLLGKFELSGIPPAPRGVPQIEVTFDIDANGILNVSAVEKGTGKSNKIT
ITNDKGRLSKEDIDKMVAEAEKFKAEDEQEAQRVQAKNQLESYAFTLKNS
VSENNFKEKVGEEDARKLEAAAQDAINWLDASQAASTEEYKERQKELEGV
ANPIMSKFYGAAGGAPGAGPVPGAGAGPTGAPDNGPTVEEVD*

SSA4 is encoded by a non-essential gene comprising an ORF that is 1.929 kbp in size and is located on chromosome V. A published nucleotide coding sequence of SSA4 is as follows, although it will be appreciated that the sequence can be modified by degenerate substitutions to obtain alternative nucleotide sequences which encode an identical protein product:

ATGTCAAAAGCTGTTGGTATTGATTTAGGTACAACCTATTCATGTGTTGCTCATTTTGCA
AACGATAGGGTTGAAATTATCGCTAACGATCAAGGTAATAGAACGACGCCTTCTTATGTG
GCTTTTACTGACACAGAAAGGCTAATTGGTGACGCTGCGAAGAATCAAGCTGCGATGAAC
CCACATAATACAGTATTCGATGCTAAGCGTCTGATCGGACGTAAATTCGATGATCCAGAA
GTGACGAACGATGCTAAGCATTACCCATTCAAAGTGATTGACAAGGGAGGTAAACCGGTA
GTGCAAGTGGAATATAAAGGCGAGACAAAGACATTTACTCCAGAAGAAATTTCCTCAATG
ATCTTGACAAAGATGAAGGAGACTGCTGAGAACTTTTTAGGAACAGAAGTGAAAGATGCT
GTAGTAACGGTTCCAGCCTATTTCAACGATTCACAAAGGCAAGCAACAAAAGATGCCGGT
ACAATCGCGGGCTTGAACGTTCTTCGTATCATTAATGAACCTACAGCTGCCGCTATTGCG
TATGGGCTGGACAAGAAATCGCAGAAGGAGCACAACGTCTTGATCTTTGATTTAGGTGGT
GGTACTTTTGATGTCTCTCTGCTATCCATAGATGAAGGTGTCTTTGAGGTTAAGGCTACT
GCTGGTGACACTCACTTGGGTGGTGAAGATTTCGATAGTAGGCTGGTTAACTTTCTAGCC
GAGGAGTTCAAAAGAAAAAATAAAAAGGATCTAACAACTAACCAAAGGTCCCTAAGGAGG
TTAAGGACCGCCGCTGAAAGGGCCAAGAGAACTCTGTCTTCGTCTGCTCAGACATCTATA
GAAATAGATTCATTATTTGAGGGTATCGATTTCTATACTTCCATTACAAGGGCAAGATTT
GAAGAATTATGTGCTGATTTGTTTAGATCTACATTGGAGCCAGTGGAAAAAGTTTTGGCT
GATTCAAAATTAGATAAGTCACAAATTGATGAAATTGTACTTGTTGGTGGTTCAACAAGA
ATTCCAAAAGTACAAAAACTGGTTTCTGATTTTTTCAATGGTAAAGAACCAAACCGTTCG
ATTAACCCTGATGAGGCCGTCGCTTATGGTGCTGCCGTACAGGCTGCCATCTTAACGGGT
GACCAGTCGTCGACGACCCAAGATTTACTGTTGCTGGATGTTGCACCATTATCTCTAGGT
ATTGAAACTGCAGGTGGTATTATGACAAAGTTGATCCCAAGAAATTCGACTATCCCAACA
AAAAAATCGGAAGTGTTTTCCACCTACGCTGACAACCAACCTGGTGTGTTGATACAAGTT
TTTGAGGGTGAAAGGACAAGGACAAAAGACAACAATCTACTGGGTAAATTTGAGTTGAGC
GGTATTCCACCCGCTCCAAGAGGCGTACCACAAATTGAAGTTACATTTGATATCGATGCA
AATGGTATTCTGAACGTATCTGCCGTTGAAAAAGGTACTGGTAAATCTAACAAGATTACA
ATTACTAACGATAAGGGAAGATTATCGAAGGAAGATATCGATAAAATGGTTGCTGAGGCA
GAAAAGTTCAAGGCCGAAGATGAACAAGAAGCTCAACGTGTTCAAGCTAAGAATCAGCTA
GAATCGTACGCGTTTACTTTGAAAAATTCTGTGAGCGAAAATAACTTCAAGGAGAAGGTG
GGTGAAGAGGATGCCAGGAAATTGGAAGCCGCCGCCCAAGATGCTATAAATTGGTTAGAT
GCTTCGCAAGCGGCCTCCACCGAGGAATACAAGGAAAGGCAAAAGGAACTAGAAGGTGTT
GCAAACCCCATTATGAGTAAATTTTACGGAGCTGCAGGTGGTGCCCCAGGAGCAGGCCCA
GTTCCGGGTGCTGGAGCAGGCCCCACTGGAGCACCAGACAACGGCCCAACGGTTGAAGAG
GTTGATTAG

Further information on SSA4 can be obtained from the URL address http://db.yeastgenome.org/cgi-bin/singlepageformat?sgdid=S000000905.

It will be appreciated that, by “SSA4”, we include fragments or variants thereof having equivalent SSA4-like activity.

SSE1 is another S. cerevisiae helper protein of interest for the present invention and is also known as LPG3 and MSI3. Sse1p is an ATPase that is a component of the heat shock protein Hsp90 chaperone complex. It binds unfolded proteins and is a member of the heat shock protein 70 (HSP70) family. It is localized to the cytoplasm. A published protein sequence for the protein Sse1p is as follows:

MSTPFGLDLGNNNSVLAVARNRGIDIVVNEVSNRSTPSVVGFGPKNRYLGETGKNKQTSN
IKNTVANLKRIIGLDYHHPDFEQESKHFTSKLVELDDKKTGAEVRFAGEKHVFSATQLAA
MFIDKVKDTVKQDTKANITDVCIAVPPWYTEEQRYNIADAARIAGLNPVRIVNDVTAAGV
SYGIFKTDLPEGEEKPRIVAFVDIGHSSYTCSIMAFKKGQLKVLGTACDKHFGGRDFDLA
ITEHFADEFKTKYKIDIRENPKAYNRILTAAEKLKKVLSANTNAPFSVESVMNDVDVSSQ
LSREELEELVKPLLERVTEPVTKALAQAKLSAEEVDFVEIIGGTTRIPTLKQSISEAFGK
PLSTTLNQDEAIAKGAAFICAIHSPTLRVRPFKFEDIHPYSVSYSWDKQVEDEDHMEVFP
AGSSFPSTKLITLNRTGDFSMAASYTDITQLPPNTPEQIANWEITGVQLPEGQDSVPVKL
KLRCDPSGLHTIEEAYTIEDIEVEEPIPLPEDAPEDAEQEFKKVTKTVKKDDLTIVAHTF
GLDAKKLNELIEKENEMLAQDKLVAETEDRKNTLEEYIYTLRGKLEEEYAPFASDAEKTK
LQGMLNKAEEWLYDEGFDSIKAKYIAKYEELASLGNIIRGRYLAKEEEKKQAIRSKQEAS
QMAAMAEKLAAQRKAEAEKKEEKKDTEGDVDMD*

SSE1 is encoded by a non-essential gene comprising an ORF that is 2.082 kbp in size and is located on chromosome XVI. A published nucleotide coding sequence of SSE1 is as follows, although it will be appreciated that the sequence can be modified by degenerate substitutions to obtain alternative nucleotide sequences which encode an identical protein product:

ATGAGTACTCCATTTGGTTTAGATTTAGGTAACAATAACTCTGTCCTTGCCGTTGCTAGA
AACAGAGGTATCGACATTGTCGTTAATGAAGTCTCTAACCGTTCCACCCCATCTGTTGTT
GGTTTTGGTCCAAAGAACAGATACTTGGGTGAAACTGGTAAGAACAAGCAGACTTCCAAC
ATCAAGAACACTGTCGCCAACTTGAAAAGAATTATTGGTTTGGATTACCACCATCCAGAT
TTCGAGCAAGAATCTAAGCACTTCACCTCTAAGTTGGTTGAATTGGATGACAAGAAGACT
GGTGCCGAAGTTAGATTCGCTGGTGAGAAACATGTTTTTTCAGCTACTCAACTAGCTGCC
ATGTTCATCGACAAAGTCAAGGACACCGTCAAGCAGGACACAAAGGCAAATATTACCGAT
GTTTGTATTGCTGTCCCACCTTGGTACACCGAAGAACAACGTTACAACATTGCTGATGCT
GCTAGAATTGCTGGTTTGAACCCTGTTAGAATTGTCAACGACGTTACTGCTGCCGGTGTT
TCTTACGGTATCTTCAAGACTGATTTGCCTGAAGGCGAAGAAAAGCCAAGAATTGTTGCC
TTTGTTGATATTGGTCACTCTTCCTACACCTGTTCTATCATGGCCTTCAAGAAGGGTCAA
TTGAAAGTCTTAGGAACTGCCTGCGACAAGCATTTTGGTGGTAGGGACTTCGATTTGGCT
ATAACAGAACATTTCGCCGATGAGTTCAAAACTAAATACAAGATTGACATCAGAGAAAAT
CCAAAGGCTTACAACAGAATTCTAACTGCTGCTGAAAAGTTGAAGAAAGTTTTGTCTGCT
AATACTAATGCCCCATTCTCTGTTGAATCCGTCATGAACGACGTTGATGTTTCCTCTCAA
TTATCTCGTGAAGAATTAGAAGAATTGGTCAAGCCATTGTTGGAACGTGTTACTGAACCA
GTTACCAAAGCTTTAGCTCAAGCCAAATTATCTGCTGAAGAAGTTGATTTTGTTGAAATT
ATTGGTGGTACTACTCGTATCCCAACATTGAAACAATCCATTTCTGAAGCCTTCGGCAAG
CCATTGTCCACCACTTTGAACCAAGATGAAGCCATCGCCAAGGGTGCCGCCTTTATTTGC
GCCATTCACTCTCCAACTCTAAGAGTTAGACCATTCAAGTTTGAGGATATCCATCCTTAC
TCTGTCTCTTACTCTTGGGACAAGCAAGTTGAGGACGAAGACCACATGGAAGTTTTCCCA
GCTGGTTCATCCTTCCCATCTACTAAATTGATCACTTTGAACCGTACGGGTGACTTTTCA
ATGGCTGCTAGCTACACTGACATCACACAGTTACCACCAAACACTCCAGAACAAATCGCT
AACTGGGAGATCACTGGTGTTCAATTACCAGAAGGTCAAGACTCTGTTCCTGTTAAGTTA
AAGTTGAGATGCGACCCCTCTGGTTTACACACAATTGAAGAGGCTTACACTATTGAAGAT
ATTGAAGTTGAAGAACCTATTCCATTACCAGAAGATGCTCCAGAAGATGCTGAGCAAGAA
TTTAAGAAGGTTACTAAAACTGTAAAGAAGGATGACTTAACCATCGTTGCACACACCTTT
GGCCTAGACGCTAAAAAGTTGAATGAATTAATTGAAAAAGAAAATGAAATGCTTGCTCAA
GATAAGCTAGTTGCTGAGACAGAAGACCGTAAGAACACTCTTGAAGAGTACATCTACACA
TTGCGTGGTAAGTTGGAAGAAGAGTATGCTCCATTTGCTTCCGATGCTGAAAAGACGAAG
TTACAAGGTATGTTAAACAAGGCCGAAGAGTGGTTATACGATGAAGGTTTCGATTCCATC
AAAGCTAAGTACATTGCCAAATACGAAGAATTGGCTTCTCTAGGTAACATTATTAGAGGT
AGATACTTGGCTAAAGAAGAAGAAAAGAAGCAAGCTATAAGATCTAAGCAAGAAGCATCC
CAAATGGCTGCTATGGCTGAAAAGTTGGCTGCTCAAAGAAAGGCAGAAGCTGAAAAGAAG
GAAGAAAAGAAGGACACTGAAGGTGATGTTGACATGGACTAA

Further information on SSE1 can be obtained from the URL address http://db.yeastgenome.org/cgi-bin/singlepageformat?sgdid=S000006027.

It will be appreciated that, by “SSE1”, we include fragments or variants thereof having equivalent SSE1-like activity.

SSE2 is another S. cerevisiae helper protein of interest for the present invention. Sse2p is a member of the heat shock protein 70 (HSP70) family. It may be involved in protein folding and is localised to the cytoplasm. It is highly homologous to the heat shock protein Sse1p. A published protein sequence for the protein Sse2p is as follows:

MSTPFGLDLGNNNSVLAVARNRGIDVVVNEVSNRSTPSLVGFGPRNRYLGESGKTKQTSN
VKNTVENLKRIIGLKFKDPEFDIENKFFTSKLVQLKNGKVGVEVEFGGKTHVFSATQLTA
MFIDKVKHTVQEETKSSITDVCLAVPVWYSEEQRYNIADAARIAGLNPVRIVNDVTAAAV
SYGVFKNDLPGPEEKPRIIGLVDIGHSTYTCSIMAFRKGEMKVLGTAYDKHFGGRDFDRA
ITEHFADQFKDKYKIDIRKNPKAYNRILIAAEKLKKVLSANTTAPFSVESVMDDIDVSSQ
LSREELEELVEPLLKRVTYPITNALAQAKLTVNDIDFVEIIGGTTRIPVLKKSISDVFGK
PLSSTLNQDEAVAKGAAFICAIHSPTLRVRPFKFEDIDPYSVSYTWDKQVDDEDRLEVFP
ANSSYPSTKLITLHRTGDFSMKAVYTHPSKLPKGTSTTIAKWSFTGVKVPKDQDFIPVKV
KLRCDPSGLHIIENAYTTEDITVQEPVPLPEDAPEDAEPQFKEVTKTIKKDVLGMTAKTF
ALNPVELNDLIEKENELRNQDKLVAETEDRKNALEEYIYTLRAKLDDEYSDFASDAEKEK
LKNMLATTENWLYGDGDDSTKAKYIAKYEELASLGNIIRGRYLAKEEEKRQALRANQETS
KMNDIAEKLAEQRRARAASDDSDDNNDENMDLD*

SSE2 is encoded by a non-essential gene comprising an ORF that is 2.082 kbp in size and is located on chromosome II. A published nucleotide coding sequence of SSE2 is as follows, although it will be appreciated that the sequence can be modified by degenerate substitutions to obtain alternative nucleotide sequences which encode an identical protein product:

ATGAGCACTCCATTTGGCTTAGATTTAGGTAACAATAACTCAGTACTAGCAGTTGCCAGA
AATAGGGGTATTGATGTCGTTGTCAATGAAGTTTCTAATAGGTCTACACCATCCTTGGTC
GGCTTTGGCCCCAGAAATAGGTACTTAGGTGAATCTGGTAAAACTAAGCAAACATCGAAT
GTTAAAAACACTGTGGAAAACTTGAAAAGAATCATTGGACTAAAGTTCAAAGACCCTGAA
TTTGATATCGAGAATAAGTTCTTCACTTCGAAATTGGTACAGCTAAAAAATGGTAAAGTT
GGTGTGGAAGTGGAGTTCGGCGGTAAAACACACGTATTTTCAGCTACTCAACTGACTGCT
ATGTTCATTGATAAGGTGAAGCACACCGTTCAAGAGGAAACGAAGTCATCAATTACCGAT
GTCTGCCTCGCAGTTCCTGTATGGTATTCGGAAGAACAACGTTATAACATAGCCGATGCT
GCCAGAATTGCAGGATTAAATCCTGTAAGGATTGTCAACGATGTGACTGCAGCCGCCGTT
TCGTACGGCGTCTTCAAGAATGATCTGCCAGGTCCTGAAGAAAAGCCAAGAATCATTGGC
TTAGTGGACATTGGGCATTCTACCTACACCTGTTCTATTATGGCTTTCCGCAAAGGCGAA
ATGAAAGTATTAGGTACTGCTTATGACAAGCACTTTGGTGGTAGAGATTTCGATCGCGCA
ATCACAGAACATTTTGCTGATCAGTTTAAGGACAAGTACAAGATTGACATTAGGAAAAAT
CCGAAAGCTTATAACAGAATTTTAATCGCTGCTGAAAAATTAAATAAAGTGCTTTCTGCG
AACACTACTGCCCCCTTCTCCGTTGAATCTGTTATGGATGATATCGACGTTTCCTCTCAA
TTGAGCCGTGAAGAGCTGGAAGAATTAGTAGAGCCCTTGTTGAAGCGTGTGACGTATCCA
ATCACCAATGCATTGGCTCAAGCTAAATTAACTGTCAATGATATTGACTTCGTAGAAATA
ATTGGTGGTACAACCCGTATCCCAGTTTTAAAGAAGTCAATTTCTGATGTTTTTGGAAAA
CCTTTGTCATCTACTTTAAATCAAGACGAAGCTGTGGCCAAGGGGGCCGCTTTCATATGT
GCCATTCACTCTCCAACTTTAAGGGTCAGGCCGTTTAAATTTGAAGATATTGATCCGTAT
TCAGTGTCATACACTTGGGATAAGCAGGTCGATGACGAAGACCGTTTGGAAGTATTCCCT
GCTAATTCATCATATCCATCAACTAAACTAATTACTTTACATCGTACTGGAGATTTCAGC
ATGAAAGCGGTGTACACTCATCCTTCGAAACTGCCAAAAGGTACTTCCACCACTATTGCA
AAATGGAGCTTCACTGGGGTCAAGGTTCCTAAAGATCAAGATTTTATTCCTGTAAAGGTC
AAGTTAAGATGCGATCCTTCCGGCTTGCATATTATCGAGAACGCTTACACAACGGAAGAT
ATTACGGTTCAAGAGCCAGTGCCTTTACCGGAAGACGCACCAGAAGATGCCGAGCCCCAG
TTTAAAGAAGTTACTAAAACAATTAAGAAAGATGTGCTAGGTATGACTGCAAAAACATTC
GCGCTAAACCCGGTTGAGTTGAACGATCTAATTGAAAAAGAGAATGAATTAAGAAACCAG
GATAAGTTAGTTGCCGAAACCGAGGATCGCAAAAATGCCCTTGAAGAGTATATTTATACC
CTTCGTGCCAAACTCGATGATGAATACTCCGATTTTGCGTCTGACGCAGAAAAAGAAAAG
CTAAAAAACATGTTAGCCACTACTGAAAATTGGTTATATGGTGATGGTGACGATTCTACC
AAGGCAAAATACATTGCTAAATATGAGGAGCTGGCATCGTTGGGGAATATTATTAGAGGT
AGATATTTAGCAAAGGAGGAAGAAAAAAGACAAGCACTCAGAGCGAATCAAGAAACTTCT
AAAATGAATGATATTGCTGAAAAATTGGCTGAGCAAAGAAGGGCACGCGCTGCAAGTGAT
GATAGCGATGACAACAATGATGAAAACATGGACCTTGATTAA

Further information on SSE2 can be obtained from the URL address http://db.yeastgenome.org/cgi-bin/singlepageformat?sgdid=S000000373.

It will be appreciated that, by “SSE2”, we include fragments or variants thereof having equivalent SSE2-like activity.

SSB1 is another S. cerevisiae helper protein of interest for the present invention and is also known as YG101. Ssb1p is a cytoplasmic ATPase that is a ribosome-associated molecular chaperone. It may be involved in the folding of newly-synthesized polypeptide chains and is a member of the heat shock protein 70 (HSP70) family. It interacts with the phosphatase subunit Reg1p. A published protein sequence for the protein Ssb1p is as follows:

MAEGVFQGAIGIDLGTTYSCVATYESSVEIIANEQGNRVTPSFVAFTPEERLIGDAAKNQ
AALNPRNTVFDAKRLIGRRFDDESVQKDMKTWPFKVIDVDGNPVIEVQYLEETKTFSPQE
ISAMVLTKMKEIAEAKIGKKVEKAVITVPAYFNDAQRQATKDAGAISGLNVLRIINEPTA
AAIAYGLGAGKSEKERHVLIFDLGGGTFDVSLLHIAGGVYTVKSTSGNTHLGGQDFDTNL
LEHFKAEFKKKTGLDISDDARALRRLRTAAERAKRTLSSVTQTTVEVDSLFDGEDFESSL
TRARFEDLNAALFKSTLEPVEQVLKDAKISKSQIDEVVLVGGSTRIPKVQKLLSDFFDGK
QLEKSINPDEAVAYGAAVQGAILTGQSTSDETKDLLLLDVAPLSLGVGMQGDMFGIVVPR
NTTVPTIKRRTFTTCADNQTTVQFPVYQGERVNCKENTLLGEFDLKNIPMMPAGEPVLEA
IFEVDANGILKVTAVEKSTGKSSNITISNAVGRLSSEEIEKMVNQAEEFKAADEAFAKKH
EARQRLESYVASIEQTVTDPVLSSKLKRGSKSKIEAALSDALAALQIEDPSADELRKAEV
GLKRVVTKAMSSR*

SSB1 is encoded by a non-essential gene comprising an ORF that is 1.842 kbp in size and is located on chromosome IV. A published nucleotide coding sequence of SSB1 is as follows, although it will be appreciated that the sequence can be modified by degenerate substitutions to obtain alternative nucleotide sequences which encode an identical protein product:

ATGGCTGAAGGTGTTTTCCAAGGTGCTATCGGTATCGATTTAGGTACAACCTACTCTTGT
GTTGCTACTTACGAATCCTCCGTTGAAATTATTGCCAACGAACAAGGTAACAGAGTCACC
CCATCTTTCGTTGCTTTCACTCCAGAAGAAAGATTGATTGGTGATGCTGCCAAGAACCAA
GCTGCTTTGAACCCAAGAAACACTGTCTTCGATGCTAAGCGTTTGATTGGTAGAAGATTC
GACGACGAATCTGTTCAAAAGGACATGAAGACCTGGCCTTTCAAGGTTATCGACGTCGAT
GGTAACCCAGTCATCGAAGTCCAATACTTGGAAGAAACCAAGACTTTCTCCCCACAAGAA
ATTTCCGCTATGGTTTTGACCAAGATGAAGGAAATTGCTGAAGCTAAGATTGGTAAGAAG
GTTGAAAAGGCCGTCATTACTGTCCCAGCTTACTTTAACGACGCTCAAAGACAAGCTACC
AAGGATGCCGGTGCCATTTCTGGTTTGAACGTTTTGCGTATCATCAACGAACCTACTGCC
GCTGCTATTGCTTACGGTCTAGGTGCTGGTAAGTCCGAAAAGGAAAGACATGTTTTGATT
TTCGATTTGGGTGGTGGTACTTTCGATGTTTCCTTGTTGCACATTGCTGGTGGTGTTTAC
ACTGTTAAATCTACTTCCGGTAACACTCACTTGGGTGGTCAAGATTTCGACACCAACTTG
TTGGAACACTTCAAGGCTGAATTCAAGAAGAAGACTGGTTTGGACATCTCCGACGATGCC
AGAGCTTTGAGAAGATTGAGAACTGCTGCTGAAAGAGCTAAGAGAACCTTATCTTCTGTC
ACTCAAACTACCGTTGAAGTTGACTCTTTGTTTGACGGTGAAGATTTCGAATCCTCTTTG
ACTAGAGCTAGATTTGAAGACTTGAACGCCGCATTGTTCAAGTCTACTTTGGAACCTGTT
GAACAAGTTTTGAAGGATGCTAAGATCTCTAAGTCTCAAATCGACGAAGTTGTCTTGGTT
GGTGGTTCCACCAGAATTCCAAAGGTCCAAAAGTTGTTGTCTGACTTCTTTGACGGTAAG
CAATTGGAAAAATCTATTAACCCAGATGAAGCTGTTGCTTACGGTGCTGCTGTTCAAGGT
GCTATCTTGACCGGCCAATCCACATCTGACGAAACCAAGGACTTGTTGTTGTTAGATGTT
GCTCCATTATCTCTAGGTGTTGGTATGCAAGGTGACATGTTCGGTATCGTTGTTCCAAGA
AACACTACTGTTCCAACCATCAAGAGAAGAACCTTTACTACATGTGCTGACAACCAAACC
ACCGTTCAATTCCCAGTCTACCAAGGTGAACGTGTTAACTGTAAAGAAAACACTTTGTTG
GGTGAATTCGACTTGAAGAACATCCCAATGATGCCAGCTGGTGAACCAGTCTTGGAAGCT
ATCTTCGAAGTTGATGCTAACGGTATCTTGAAGGTTACTGCCGTCGAAAAGTCTACCGGT
AAGTCTTCTAACATCACTATCTCTAACGCTGTTGGTAGATTGTCTTCTGAAGAAATTGAA
AAGATGGTTAACCAAGCTGAAGAGTTCAAGGCTGCCGATGAAGCTTTTGCCAAGAAGCAC
GAAGCTAGACAAAGATTGGAATCCTACGTTGCCTCCATCGAACAAACTGTCACTGACCCA
GTCTTGTCTTCTAAATTGAAGAGAGGTTCCAAGTCCAAGATTGAAGCTGCTTTGTCCGAT
GCTTTGGCTGCTTTGCAAATCGAAGACCCATCTGCTGATGAATTGAGAAAGGCTGAAGTT
GGTTTGAAGAGAGTTGTCACCAAGGCCATGTCTTCTCGTTAA

Further information on SSB1 can be obtained from the URL address http://db.yeastgenome.org/cgi-bin/singlepageformat?sgdid=S000002388.

It will be appreciated that, by “SSB1”, we include fragments or variants thereof having equivalent SSB1-like activity.

SSB2 is another S. cerevisiae helper protein of interest for the present invention. Ssb2p is a cytoplasmic ATPase that is a ribosome-associated molecular chaperone. It may be involved in the folding of newly-synthesized polypeptide chains. It is a member of the heat shock protein 70 (HSP70) family and is a homolog of SSB1. A published protein sequence for the protein Ssb2p is as follows:

MAEGVFQGAIGIDLGTTYSCVATYESSVEIIANEQGNRVTPSFVAFTPQERLIGDAAKNQ
AALNPRNTVFDAKRLIGRRFDDESVQKDMKTWPFKVIDVDGNPVIEVQYLEETKTFSPQE
ISAMVLTKMKEIAEAKIGKKVEKAVITVPAYFNDAQRQATKDAGAISGLNVLRIINEPTA
AAIAYGLGAGKSEKERHVLIFDLGGGTFDVSLLHIAGGVYTVKSTSGNTHLGGQDFDTNL
LEHFKAEFKKKTGLDISDDARALRRLRTAAERAKRTLSSVTQTTVEVDSLFDGEDFESSL
TRARFEDLNAALFKSTLEPVEQVLKDAKISKSQIDEVVLVGGSTRIPKVQKLLSDFFDGK
QLEKSINPDEAVAYGAAVQGAILTGQSTSDETKDLLLLDVAPLSLGVGMQGDIFGIVVPR
NTTVPTIKRRTFTTVSDNQTTVQFPVYQGERVNCKENTLLGEFDLKNIPMMPAGEPVLEA
IFEVDANGILKVTAVEKSTGKSSNITISNAVGRLSSEEIEKMVNQAEEFKAADEAFAKKH
EARQRLESYVASIEQTVTDPVLSSKLKRGSKSKIEAALSDALAALQIEDPSADELRKAEV
GLKRVVTKAMSSR*

SSB2 is encoded by a non-essential gene comprising an ORF that is 1.842 kbp in size and is located on chromosome XIV. A published nucleotide coding sequence of SSB2 is as follows, although it will be appreciated that the sequence can be modified by degenerate substitutions to obtain alternative nucleotide sequences which encode an identical protein product:

ATGGCTGAAGGTGTTTTCCAAGGTGCTATCGGTATCGATTTAGGTACAACATACTCTTGT
GTTGCTACTTATGAATCTTCCGTTGAAATTATTGCCAACGAACAAGGTAACAGAGTTACT
CCATCTTTCGTTGCCTTCACCCCACAGGAAAGATTGATCGGTGATGCTGCCAAGAACCAA
GCTGCTTTGAACCCAAGAAACACTGTTTTTGATGCTAAGCGTTTGATTGGTAGAAGATTC
GACGACGAGTCTGTCCAAAAGGACATGAAGACCTGGCCTTTCAAGGTTATCGACGTCGAT
GGTAACCCAGTCATTGAAGTCCAATACTTGGAAGAAACCAAGACTTTCTCCCCACAAGAA
ATTTCCGCTATGGTCTTGACCAAGATGAAGGAAATTGCTGAAGCTAAGATTGGTAAGAAG
GTTGAAAAGGCTGTCATTACTGTCCCAGCTTACTTTAACGATGCCCAAAGACAAGCTACC
AAGGATGCCGGTGCCATTTCTGGTTTGAACGTTTTGCGTATCATCAACGAACCTACTGCC
GCTGCTATTGCTTACGGTCTAGGTGCTGGTAAGTCCGAAAAGGAAAGACATGTTTTGATT
TTCGATTTGGGTGGTGGTACTTTCGATGTTTCCTTGTTGCACATTGCTGGTGGTGTTTAC
ACTGTTAAATCTACTTCCGGTAACACTCACTTGGGTGGTCAAGATTTCGACACCAACTTG
TTGGAACACTTCAAGGCTGAATTCAAGAAGAAGACTGGTTTGGACATCTCCGACGATGCC
AGAGCTTTGAGAAGATTGAGAACTGCTGCTGAAAGAGCTAAGAGAACCTTATCTTCTGTC
ACTCAAACTACCGTTGAAGTTGACTCTTTGTTTGACGGTGAAGATTTCGAATCCTCTTTG
ACTAGAGCTAGATTTGAAGACTTGAACGCCGCATTGTTCAAGTCTACTTTGGAACCTGTT
GAACAAGTTTTGAAGGATGCTAAGATCTCTAAGTCTCAAATCGACGAAGTTGTCTTGGTT
GGTGGTTCTACCAGAATTCCAAAGGTCCAAAAGTTGTTGTCTGACTTCTTTGACGGTAAG
CAATTGGAAAAATCTATTAACCCAGATGAAGCTGTTGCTTACGGTGCTGCTGTTCAAGGT
GCTATCTTGACTGGCCAATCCACATCTGACGAAACCAAGGACTTGTTGTTGTTAGATGTT
GCTCCATTATCTCTAGGTGTTGGTATGCAAGGTGACATTTTCGGTATTGTTGTCCCAAGA
AACACAACTGTTCCAACCATCAAGAGAAGAACCTTCACAACTGTCAGTGACAACCAAACC
ACCGTTCAATTCCCAGTCTACCAAGGTGAACGTGTCAACTGTAAAGAAAACACTTTGTTG
GGTGAATTCGACTTGAAGAACATCCCAATGATGCCAGCTGGTGAACCAGTCTTGGAAGCT
ATCTTCGAAGTTGATGCTAACGGTATCTTGAAGGTTACTGCCGTCGAAAAGTCTACCGGT
AAGTCTTCTAACATCACTATCTCCAACGCTGTCGGTAGATTGTCTTCTGAAGAAATTGAA
AAGATGGTTAACCAAGCCGAAGAGTTCAAGGCTGCTGATGAAGCTTTTGCTAAGAAGCAC
GAAGCTAGACAAAGACTAGAATCCTACGTCGCTTCCATCGAACAAACCGTCACTGACCCA
GTCTTGTCTTCTAAATTGAAGAGAGGTTCCAAGTCCAAGATCGAAGCTGCTTTGTCCGAT
GCTTTGGCTGCTTTGCAAATCGAAGACCCATCCGCTGATGAGTTGAGAAAGGCAGAAGTT
GGTTTGAAGAGAGTTGTCACCAAGGCCATGTCTTCTCGTTAA

Further information on SSB2 can be obtained from the URL address http://db.yeastgenome.org/cgi-bin/singlepageformat?sgdid=S000005153.

It will be appreciated that, by “SSB2”, we include fragments or variants thereof having equivalent SSB2-like activity.

ECM10 is another S. cerevisiae helper protein of interest for the present invention and is also known as SSC3. Ecm10p is a heat shock protein of the Hsp70 family, which is localised in mitochondrial nucleoids. It is thought to play a role in protein translocation. It interacts with Mge1p in an ATP-dependent manner. Over-expression has been shown to induce extensive mitochondrial DNA aggregations. A published protein sequence for the protein Ecm10p is as follows:

MLPSWKAFKAHNILRILTRFQSTKIPDAVIGIDLGTTNSAVAIMEGKVPRIIENAEGSRT
TPSVVAFTKDGERLVGEPAKRQSVINSENTLFATKRLIGRRFEDAEVQRDINQVPFKIVK
HSNGDAWVEARNRTYSPAQIGGFILNKMKETAEAYLAKSVKNAVVTVPAYFNDAQRQATK
DAGQIIGLNVLRVVNEPTAAALAYGLDKSEPKVIAVFDLGGGTFDISILDIDNGIFEVKS
TNGDTHLGGEDFDIYLLQEIISHFKKETGIDLSNDRMAVQRIREAAEKAKIELSSTLSTE
INLPFITADAAGPKHIRMPFSRVQLENITAPLIDRTVDPVKKALKDARITASDISDVLLV
GGMSRMPKVADTVKKLFGKDASKAVNPDEAVALGAAIQAAVLSGEVTDVLLLDVTPLSLG
IETLGGVFTKLIPRNSTIPNKKSQIFSTAASGQTSVEVKVFQGERELVKDNKLIGNFTLA
GIPPAPKGTPQIEVTFDIDANGIINVSAKDLASHKDSSITVAGASGLSDTEIDRMVNEAE
RYKNQDRARRNAIETANKADQLANDTENSIKEFEGKLDKTDSQRLKDQISSLRELVSRSQ
AGDEVNDDDVGTKIDNLRTSSMKLFEQLYKNSDNPETKNGRENK*

ECM10 is encoded by a non-essential gene comprising an ORF that is 1.935 kbp in size and is located on chromosome V. A published nucleotide coding sequence of ECM10 is as follows, although it will be appreciated that the sequence can be modified by degenerate substitutions to obtain alternative nucleotide sequences which encode an identical protein product:

ATGTTACCATCATGGAAAGCCTTTAAAGCACATAATATACTTCGTATTCTGACCCGTTTC
CAGTCAACCAAAATTCCAGATGCAGTTATCGGTATTGATTTAGGTACTACCAATTCTGCG
GTAGCTATTATGGAAGGTAAAGTTCCGAGAATTATCGAAAATGCAGAAGGCTCAAGAACT
ACTCCGTCTGTAGTGGCTTTCACTAAAGACGGAGAACGTTTAGTTGGTGAGCCAGCCAAA
CGACAATCCGTCATAAACTCAGAAAACACTTTGTTTGCTACTAAGCGTTTAATCGGCCGC
CGTTTCGAGGACGCTGAAGTCCAAAGAGATATTAATCAGGTTCCTTTCAAAATCGTCAAG
CATTCTAATGGAGATGCCTGGGTAGAGGCTAGAAACAGAACGTACTCCCCCGCCCAAATA
GGAGGTTTTATCTTAAATAAAATGAAGGAAACAGCGGAGGCTTACTTAGCGAAGAGCGTC
AAAAATGCTGTTGTCACCGTTCCTGCTTACTTCAATGATGCCCAAAGACAAGCTACTAAA
GACGCAGGACAAATTATTGGGCTTAATGTATTACGTGTTGTCAACGAACCAACAGCTGCT
GCCCTAGCTTACGGTCTAGATAAATCAGAGCCAAAAGTCATTGCTGTTTTCGACTTGGGC
GGTGGTACTTTCGATATTTCAATCCTGGACATCGATAACGGTATCTTTGAGGTTAAATCT
ACCAATGGTGACACCCATTTGGGTGGCGAAGATTTTGACATTTATTTGTTGCAAGAAATT
ATTTCTCATTTCAAGAAAGAAACCGGTATCGATTTGAGTAATGACCGTATGGCTGTCCAA
AGAATAAGAGAAGCCGCTGAAAAGGCTAAAATCGAACTGTCTTCTACACTCTCTACAGAA
ATAAACTTGCCTTTCATAACTGCTGATGCTGCAGGCCCAAAGCATATTCGTATGCCCTTT
TCTAGGGTTCAGCTTGAGAATATAACCGCCCCATTGATTGATAGAACGGTTGATCCTGTC
AAAAAAGCACTGAAAGACGCAAGAATTACCGCCTCAGATATATCGGATGTTTTATTAGTT
GGTGGTATGTCAAGGATGCCCAAGGTTGCAGATACTGTAAAGAAATTATTCGGTAAGGAT
GCATCAAAAGCTGTTAACCCTGATGAAGCAGTCGCTTTAGGGGCCGCTATACAGGCTGCG
GTCTTGTCTGGTGAAGTTACCGATGTTTTGTTGCTAGATGTCACTCCCCTATCATTGGGT
ATTGAAACTTTAGGAGGAGTTTTTACAAAATTAATCCCAAGAAATTCTACAATTCCCAAT
AAGAAATCTCAAATTTTTTCAACTGCGGCATCAGGTCAAACATCGGTGGAAGTTAAAGTT
TTCCAAGGTGAGAGGGAGTTAGTCAAGGATAACAAATTAATAGGTAATTTTACTCTTGCG
GGCATTCCTCCAGCTCCAAAAGGTACCCCACAAATTGAAGTCACTTTTGATATCGATGCG
AACGGCATCATCAACGTTTCAGCAAAAGATCTCGCCAGCCACAAAGACTCTTCCATCACT
GTTGCCGGAGCGTCTGGGCTATCTGATACGGAGATTGATCGAATGGTTAATGAAGCGGAA
AGATATAAAAATCAGGATAGAGCCAGAAGGAATGCCATCGAAACCGCTAACAAAGCTGAC
CAGCTAGCTAATGACACAGAAAATTCCATTAAGGAATTCGAAGGTAAGCTAGATAAAACT
GATTCTCAAAGACTAAAAGATCAAATTTCATCCTTAAGGGAATTGGTTTCTCGGAGTCAA
GCTGGAGATGAGGTTAATGATGACGATGTTGGAACAAAAATTGACAATTTGCGAACTTCA
TCGATGAAACTTTTTGAACAGTTATACAAGAACAGTGACAATCCTGAAACTAAGAACGGG
AGAGAAAATAAATAA

Further information on ECM10 can be obtained from the URL address http://db.yeastgenome.org/cgi-bin/singlepageformat?sgdid=S000000756.

It will be appreciated that, by “ECM10”, we include fragments or variants thereof having equivalent ECM10-like activity.

MDJ1 is another S. cerevisiae helper protein of interest for the present invention. Mdj1p is a protein involved in folding of mitochondrially synthesised proteins in the mitochondrial matrix. It localises to the mitochondrial inner membrane and is a member of the DnaJ family of molecular chaperones. A published protein sequence for the protein Mdj1p is as follows:

MAFQQGVLSRCSGVFRHHVGHSRHINNILYRHAIAFASIAPRIPKSSFHTSAIRNNEAFK
DPYDTLGLKKSATGAEIKKAYYKLAKKYHPDINKEPDAEKKFHDLQNAYEILSDETKRQQ
YDQFGPAAFGGGGAAGGAGGGSGSPFGSQFHDFSGFTSAGGSPFGGINFEDLFGAAFGGG
GRGSGGASRSSSMFRQYRGDPIEIVHKVSFKDAVFGSKNVQLRFSALDPCSTCSGTGMKP
NTHKVSCSTCHGTGTTVHIRGGFQMMSTCPTCNGEGTMKRPQDNCTKCHGEGVQVNRAKT
ITVDLPHGLQDGDVVRIPGQGSYPDIAVEADLKDSVKLSRGDILVRIRVDKDPNFSIKNK
YDIWYDKEIPITTAALGGTVTIPTVEGQKIRIKVAPGTQYNQVISIPNMGVPKTSTIRGD
MKVQYKIVVKKPQSLAEKCLWEALADVTNDDMAKKTMQPGTAAGTAINEEILKKQKQEEE
KHAKKDDDNTLKRLENFITNTFRKIKGDKKN*

MDJ1 is encoded by a non-essential gene comprising an ORF that is 1.536 kbp in size and is located on chromosome VI. A published nucleotide coding sequence of MDJ1 is as follows, although it will be appreciated that the sequence can be modified by degenerate substitutions to obtain alternative nucleotide sequences which encode an identical protein product:

ATGGCTTTCCAACAAGGTGTATTGTCAAGGTGTTCCGGTGTCTTTAGACACCATGTGGGA
CATTCTCGCCATATCAATAATATTCTTTATAGACATGCCATCGCGTTTGCATCCATCGCT
CCACGAATACCAAAATCTAGCTTCCATACTTCTGCAATCAGAAACAACGAAGCATTCAAG
GACCCGTACGATACTTTAGGCTTGAAGAAATCTGCTACAGGTGCGGAAATCAAAAAAGCA
TACTACAAACTGGCAAAGAAGTACCACCCGGATATCAACAAGGAACCGGATGCTGAGAAG
AAATTCCACGATTTACAGAACGCTTATGAAATTCTGTCAGACGAAACGAAGAGGCAGCAG
TACGATCAATTTGGGCCCGCTGCCTTCGGCGGCGGCGGTGCCGCTGGAGGTGCCGGTGGT
GGTAGTGGCTCTCCCTTTGGTTCCCAATTTCATGATTTCTCAGGATTCACCAGTGCAGGC
GGCTCGCCATTTGGCGGTATCAATTTTGAAGACCTGTTTGGTGCTGCATTTGGTGGTGGT
GGCCGCGGTAGCGGTGGCGCAAGCAGGTCGTCATCTATGTTCAGACAATATAGGGGCGAC
CCAATCGAGATTGTCCATAAAGTGTCTTTCAAGGACGCAGTGTTTGGGTCCAAGAACGTT
CAGTTAAGATTCTCTGCGCTGGACCCTTGTAGTACCTGTTCAGGGACGGGAATGAAACCA
AACACGCATAAGGTCAGTTGTAGCACTTGTCACGGAACAGGAACCACTGTTCACATTAGG
GGCGGATTTCAGATGATGTCGACTTGTCCTACTTGCAACGGTGAAGGTACCATGAAACGG
CCTCAGGACAATTGTACCAAGTGCCATGGTGAGGGTGTTCAGGTCAACAGGGCAAAGACA
ATTACGGTGGACTTGCCACATGGATTACAGGACGGCGACGTGGTCAGGATCCCTGGCCAA
GGCTCATACCCTGACATCGCTGTAGAGGCGGACTTGAAAGATTCAGTCAAGTTATCAAGA
GGTGATATTTTGGTGAGAATTCGTGTCGACAAGGATCCCAACTTTTCGATAAAGAACAAG
TACGATATTTGGTACGACAAGGAGATTCCTATAACCACAGCTGCACTTGGTGGTACTGTC
ACTATCCCCACTGTGGAGGGACAAAAGATCAGGATAAAGGTCGCTCCAGGGACTCAATAC
AATCAAGTGATATCCATTCCTAACATGGGTGTTCCTAAAACATCAACCATTCGCGGTGAT
ATGAAAGTCCAGTACAAGATCGTTGTTAAGAAACCGCAATCGCTGGCAGAAAAATGCTTG
TGGGAGGCACTGGCAGATGTCACCAACGATGACATGGCCAAGAAAACCATGCAACCGGGC
ACAGCCGCGGGTACAGCCATTAATGAAGAGATACTGAAGAAACAAAAACAAGAAGAGGAA
AAACACGCAAAAAAGGATGACGACAACACTTTGAAGAGACTAGAAAATTTCATTACCAAC
ACATTCAGGAAGATCAAAGGTGACAAAAAAAATTAA

Further information on MDJ1 can be obtained from the URL address http://db.yeastgenome.org/cgi-bin/singlepageformat?sgdid=S000001878.

It will be appreciated that, by “MDJ1”, we include fragments or variants thereof having equivalent MDJ1-like activity.

MDJ2 is another S. cerevisiae helper protein of interest for the present invention. Mdj2p is a protein of the mitochondrial inner membrane. Its function partially overlaps that of Mdj1p, which is a chaperone involved in folding of mitochondrially synthesised proteins in the mitochondrial matrix. It is a member of the DnaJ family. A published protein sequence for the protein Mdj2p is as follows:

MVLPIIIGLGVTMVALSVKSGLNAWTVYKTLSPLTIAKLNNIRIENPTA
GYRDALKFKSSLIDEELKNRLNQYQGGFAPRMTEPEALLILDISAREIN
HLDEKLLKKKHRKAMVRNHPDRGGSPYMAAKINEAKEVLERSVLLRKR*

MDJ2 is encoded by a non-essential gene comprising an ORF that is 0.441 kbp in size and is located on chromosome XIV. A published nucleotide coding sequence of MDJ2 is as follows, although it will be appreciated that the sequence can be modified by degenerate substitutions to obtain alternative nucleotide sequences which encode an identical protein product:

ATGGTTTTGCCTATAATAATTGGTTTGGGCGTGACAATGGTTGCTCTAAGTGTCAAGTCT
GGTCTCAATGCATGGACCGTCTACAAGACCCTGTCCCCTTTAACTATTGCAAAACTAAAT
AACATTCGCATAGAAAACCCGACGGCGGGCTACCGCGATGCACTTAAGTTCAAAAGCTCA
CTGATAGACGAAGAACTGAAAAATAGATTAAACCAGTACCAGGGAGGCTTTGCACCGCGA
ATGACAGAGCCCGAAGCCTTGCTCATCTTGGATATCTCCGCCAGAGAGATTAATCACTTG
GATGAAAAATTACTGAAAAAAAAGCACAGGAAGGCTATGGTTCGTAACCACCCAGACAGA
GGAGGGAGTCCCTACATGGCGGCCAAGATAAATGAGGCGAAAGAAGTTCTCGAAAGAAGT
GTTTTACTAAGAAAGAGATAA

Further information on MDJ2 can be obtained from the URL address http://db.yeastgenome.org/cgi-bin/singlepageformat?sgdid=S000005272.

It will be appreciated that, by “MDJ2”, we include fragments or variants thereof having equivalent MDJ2-like activity.

ERO1 is another S. cerevisiae helper protein of interest for the present invention. EroIp is a glycoprotein required for oxidative protein folding in the endoplasmic reticulum. A published protein sequence for the protein Ero1p is as follows:

MRLRTAIATLCLTAFTSATSNNSYIATDQTQNAFNDTHFCKVDRNDHVSPSCNVTFNELN
AINENIRDDLSALLKSDFFKYFRLDLYKQCSFWDANDGLCLNRACSVDVVEDWDTLPEYW
QPEILGSFNNDTMKEADDSDDECKFLDQLCQTSKKPVDIEDTINYCDVNDFNGKNAVLID
LTANPERFTGYGGKQAGQIWSTIYQDNCFTIGETGESLAKDAFYRLVSGFHASIGTHLSK
EYLNTKTGKWEPNLDLFMARIGNFPDRVTNMYFNYAVVAKALWKIQPYLPEFSFCDLVNK
EIKNKMDNVISQLDTKIFNEDLVFANDLSLTLKDEFRSRFKNVTKIMDCVQCDRCRLWGK
IQTTGYATALKILFEINDADEFTKQHIVGKLTKYELIALLQTFGRLSESIESVNMFEKMY
GKRLNGSENRLSSFFQNNFFNILKEAGKSIRYTIENINSTKEGKKKTNNSQSHVFDDLKM
PKAEIVPRPSNGTVNKWKKAWNTEVNNVLEAFRFIYRSYLDLPRNIWELSLMKVYKFWNK
FIGVADYVSEETREPISYKLDIQ*

ERO1 is encoded by an essential gene comprising an ORF that is 1.692 kbp in size and is located on chromosome XIII. A published nucleotide coding sequence of ERO1 is as follows, although it will be appreciated that the sequence can be modified by degenerate substitutions to obtain alternative nucleotide sequences which encode an identical protein product:

ATGAGATTAAGAACCGCCATTGCCACACTGTGCCTCACGGCTTTTACATCTGCAACTTCA
AACAATAGCTACATCGCCACCGACCAAACACAAAATGCCTTTAATGACACTCACTTTTGT
AAGGTCGACAGGAATGATCACGTTAGTCCCAGTTGTAACGTAACATTCAATGAATTAAAT
GCCATAAATGAAAACATTAGAGATGATCTTTCGGCGTTATTAAAATCTGATTTCTTCAAA
TACTTTCGGCTGGATTTATACAAGCAATGTTCATTTTGGGACGCCAACGATGGTCTGTGC
TTAAACCGCGCTTGCTCTGTTGATGTCGTAGAGGACTGGGATACACTGCCTGAGTACTGG
CAGCCTGAGATCTTGGGTAGTTTCAATAATGATACAATGAAGGAAGCGGATGATAGCGAT
GACGAATGTAAGTTCTTAGATCAACTATGTCAAACCAGTAAAAAACCTGTAGATATCGAA
GACACCATCAACTACTGTGATGTAAATGACTTTAACGGTAAAAACGCCGTTCTGATTGAT
TTAACAGCAAATCCGGAACGATTTACAGGTTATGGTGGTAAGCAAGCTGGTCAAATTTGG
TCTACTATCTACCAAGACAACTGTTTTACAATTGGCGAAACTGGTGAATCATTGGCCAAA
GATGCATTTTATAGACTTGTATCCGGTTTCCATGCCTCTATCGGTACTCACTTATCAAAG
GAATATTTGAACACGAAAACTGGTAAATGGGAGCCCAATCTGGATTTGTTTATGGCAAGA
ATCGGGAACTTTCCTGATAGAGTGACAAACATGTATTTCAATTATGCTGTTGTAGCTAAG
GCTCTCTGGAAAATTCAACCATATTTACCAGAATTTTCATTCTGTGATCTAGTCAATAAA
GAAATCAAAAACAAAATGGATAACGTTATTTCCCAGCTGGACACAAAAATTTTTAACGAA
GACTTAGTTTTTGCCAACGACCTAAGTTTGACTTTGAAGGACGAATTCAGATCTCGCTTC
AAGAATGTCACGAAGATTATGGATTGTGTGCAATGTGATAGATGTAGATTGTGGGGCAAA
ATTCAAACTACCGGTTACGCAACTGCCTTGAAAATTTTGTTTGAAATCAACGACGCTGAT
GAATTCACCAAACAACATATTGTTGGTAAGTTAACCAAATATGAGTTGATTGCACTATTA
CAGACTTTCGGTAGATTATCTGAATCTATTGAATCTGTTAACATGTTCGAAAAAATGTAC
GGGAAAAGGTTAAACGGTTCTGAAAACAGGTTAAGCTCATTCTTCCAAAATAACTTCTTC
AACATTTTGAAGGAGGCAGGCAAATCGATTCGTTACACCATAGAGAACATCAATTCCACT
AAAGAAGGAAAGAAAAAGACTAACAATTCTCAATCACATGTATTTGATGATTTAAAAATG
CCCAAAGCAGAAATAGTTCCAAGGCCCTCTAACGGTACAGTAAATAAATGGAAGAAAGCT
TGGAATACTGAAGTTAACAACGTTTTAGAAGCATTCAGATTTATTTATAGAAGCTATTTG
GATTTACCCAGGAACATCTGGGAATTATCTTTGATGAAGGTATACAAATTTTGGAATAAA
TTCATCGGTGTTGCTGATTACGTTAGTGAGGAGACACGAGAGCCTATTTCCTATAAGCTA
GATATACAATAA

Further information on ERO1 can be obtained from the URL address http://db.yeastgenome.org/cgi-bin/singlepageformat?sgdid=S000004599.

It will be appreciated that, by “ERO1”, we include fragments or variants thereof having equivalent ERO1-like activity.

ERV2 is another S. cerevisiae helper protein of interest for the present invention. Erv2p is a flavin-linked sulfhydryl oxidase localised to the endoplasmic reticulum lumen, involved in disulphide bond formation within the ER. A published protein sequence for the protein Erv2p is as follows:

MKQIVKRSHAIRIVAALGIIGLWMFFSSNELSIATPGLIKAKSGIDEVQG
AAAEKNDARLKEIEKQTIMPLMGDDKVKKEVGRASWKYFHTLLARFPDEP
TPEEREKLHTFIGLYAELYPCGECSYHFVKLIEKYPVQTSSRTAAMWA
GCHIHNKVNEYLKKDIYDCATILEDYDCGCSDSDGKRVSLEKEAKQHG*

ERV2 is encoded by a non-essential gene comprising an ORF that is 0.591 kbp in size, located on chromosome XVI. A published nucleotide coding sequence of ERV2 is as follows, although it will be appreciated that the sequence can be modified by degenerate substitutions to obtain alternative nucleotide sequences which encode an identical protein product:

ATGAAACAGATAGTCAAAAGAAGCCATGCCATCAGAATAGTTGCAGCATTAGGAATCATA
GGCCTGTGGATGTTTTTCTCGTCTAATGAACTATCCATCGCTACGCCGGGCCTAATCAAG
GCGAAGTCTGGTATAGATGAAGTGCAAGGGGCGGCTGCTGAGAAGAACGACGCTCGGTTG
AAAGAGATCGAGAAGCAAACCATTATGCCATTGATGGGCGATGACAAGGTGAAGAAGGAA
GTGGGCAGGGCGTCGTGGAAGTACTTCCATACCCTGCTGGCCCGTTTTCCGGACGAGCCT
ACTCCTGAAGAAAGAGAGAAACTGCACACGTTTATTGGGTTGTATGCAGAACTCTATCCA
TGCGGGGAATGTTCATATCACTTTGTAAAGTTGATTGAGAAGTATCCCGTACAGACATCT
AGCAGGACGGCTGCCGCAATGTGGGGATGCCACATTCACAACAAGGTGAACGAATACCTA
AAGAAAGACATATATGACTGTGCTACCATCCTGGAGGACTACGATTGTGGATGTAGTGAC
AGCGACGGTAAACGCGTGTCTCTCGAGAAGGAGGCTAAACAGCACGGTTGA

Further information on ERV2 can be obtained from the URL address http://db.yeastgenome.org/cgi-bin/singlepageformat?sgdid=S000006241.

It will be appreciated that, by “ERV2”, we include fragments or variants thereof having equivalent ERV2-like activity.

EUG1 is another S. cerevisiae helper protein of interest for the present invention. Eug1p is a protein disulphide isomerase of the endoplasmic reticulum lumen, with an overlapping function with Pdi1p. It may interact with nascent polypeptides in the ER. A published protein sequence for the protein Eug1p is as follows:

MQVTTRFISAIVSFCLFASFTLAENSARATPGSDLLVLTEKKFKSFIESHPLVLVEFFAP
WCLHSQILRPHLEEAASILKEHNVPVVQIDCEANSMVCLQQTINTYPTLKIFKNGRIFDG
QVYRGVKITDEITQYMIQLYEASVIYLNSEDEIQPYLENATLPVVINRGLTGLNETYQEV
ALDLAEDYVFLSLLDSEDKSLSIHLPNTTEPILFDGNVDSLVGNSVALTQWLKVVILPYF
TDIEPDLFPKYISSNLPLAYFFYTSEEELEDYTDLFTQLGKENRGQINFIALNSTMFPHH
VRFLNMREQFPLFAIHNMINNLKYGLPQLPEEEYAKLEKPQPLDRDMIVQLVKDYREGTA
KPIVKSEEIPKEQKSNVYKIVGKTHDDIVHDDDKDVLVKYYATWCIHSKRFAPIYEEIAN
VLASDESVRDKILIAEVDSGANDILSFPVTGYPTIALYPAGNNSKPIIFNKIRNLEDVFE
FIKESGTHHIDGQAIYDKLHQAKDSEVSTEDTVHDEL*

EUG1 is encoded by a non-essential gene comprising an ORF that is 1.554 kbp in size and is located on chromosome IV. A published nucleotide coding sequence of EUG1 is as follows, although it will be appreciated that the sequence can be modified by degenerate substitutions to obtain alternative nucleotide sequences which encode an identical protein product:

ATGCAAGTGACCACAAGATTTATATCTGCGATAGTCTCGTTTTGCCTGTTTGCTTCTTTC
ACGTTGGCTGAAAACAGCGCAAGAGCTACGCCGGGATCAGATTTACTCGTTCTAACAGAG
AAGAAATTTAAATCATTCATCGAATCTCATCCGTTAGTCCTCGTCGAGTTTTTTGCTCCA
TGGTGTTTGCATTCTCAGATCTTACGCCCTCACTTAGAAGAGGCCGCCTCTATTTTAAAG
GAGCATAACGTCCCAGTTGTTCAAATTGATTGTGAGGCTAACAGTATGGTTTGCCTGCAA
CAAACTATAAATACCTACCCAACCTTGAAAATCTTTAAAAATGGTCGTATTTTTGATGGT
CAAGTCTATCGCGGTGTCAAGATCACCGATGAAATCACTCAGTACATGATTCAGCTATAC
GAGGCTTCTGTCATTTATTTAAATTCCGAAGATGAAATCCAACCATACTTGGAAAATGCA
ACTTTACCAGTAGTAATAAACAGAGGCTTGACAGGCTTGAATGAAACGTATCAAGAAGTC
GCACTGGACCTTGCTGAGGATTACGTCTTTTTATCCCTTCTAGATTCAGAAGATAAGTCA
TTATCAATCCACTTGCCAAACACTACAGAACCAATTCTGTTTGATGGAAATGTAGACTCT
TTGGTCGGAAATTCCGTTGCTCTAACTCAGTGGTTAAAAGTGGTAATTTTACCTTACTTT
ACCGACATCGAACCTGATCTCTTCCCCAAGTACATTTCTAGCAATTTGCCGTTGGCTTAC
TTCTTTTATACTTCTGAGGAAGAATTGGAAGATTACACTGATCTTTTCACGCAGTTAGGT
AAGGAAAATCGTGGCCAAATAAATTTCATTGCATTAAACTCTACAATGTTCCCACACCAC
GTTAGATTCCTAAATATGAGAGAACAGTTCCCATTATTTGCTATCCATAATATGATCAAT
AATCTGAAATATGGTTTACCACAACTACCAGAAGAAGAGTACGCGAAATTAGAAAAACCA
CAACCACTAGACAGAGATATGATCGTTCAGTTGGTAAAAGATTACCGTGAAGGTACTGCC
AAGCCAATTGTTAAGTCAGAAGAGATTCCAAAAGAACAAAAGTCCAATGTTTATAAAATA
GTTGGGAAGACACATGACGACATTGTTCATGATGATGACAAGGATGTCCTTGTCAAATAT
TACGCGACATGGTGTATTCATAGTAAAAGGTTTGCGCCTATTTACGAAGAAATTGCAAAT
GTCTTAGCATCTGATGAATCTGTTCGCGATAAAATCTTGATCGCCGAAGTAGATTCAGGG
GCAAATGATATCTTAAGTTTTCCTGTGACAGGATATCCAACCATTGCTTTGTATCCTGCC
GGAAATAACTCTAAGCCTATTATCTTCAATAAAATTAGAAATTTGGAAGATGTTTTCGAA
TTTATCAAGGAATCAGGTACACATCACATTGACGGCCAGGCAATTTATGATAAATTGCAC
CAGGCCAAGGATTCTGAAGTGTCTACTGAAGATACCGTACATGATGAATTATAA

Further information on EUG1 can be obtained from the URL address http://db.yeastgenome.org/cgi-bin/singlepageformat?sgdid=S000002926.

It will be appreciated that, by “EUG1”, we include fragments or variants thereof having equivalent EUG1-like activity.

MPD1 is another S. cerevisiae helper protein of interest for the present invention. Mpd1p is a member of the protein disulphide isomerase (PDI) family. Its over-expression suppresses the defect in maturation of carboxypeptidase Y, and defects in other essential Pdi1p functions that can be caused by PDI1 deletion. A published protein sequence for the protein Mpd1p is as follows:

MLFLNIIKLLLGLFIMNEVKAQNFYDSDPHISELTPKSFDKAIHNTNYTS
LVEFYAPWCGHCKKLSSTFRKAAKRLDGVVQVAAVNCDLNKNKALCAKYD
VNGFPTLMVFRPPKIDLSKPIDNAKKSFSAHANEVYSGARTLAPIVDFSL
SRIRSYVKKFVRIDTLGSLLRKSPKLSVVLFSKQDKISPVYKSIALDWLG
KFDFYSISNKKLKQLTDMNPTYEKTPEIFKYLQKVIPEQRQSDKSKLVVF
DADKDKFWEYEGNSINKNDISKFLRDTFSITPNEGPFSRRSEYIAYLKTG
KKPIKKNHSSSGNKHDEL*

MPD1 is encoded by a non-essential gene comprising an ORF that is 0.957 kbp in size and is located on chromosome XV. A published nucleotide coding sequence of MPD1 is as follows, although it will be appreciated that the sequence can be modified by degenerate substitutions to obtain alternative nucleotide sequences which encode an identical protein product:

ATGTTATTTCTTAATATTATTAAGCTCCTTTTGGGACTTTTTATTATGAATGAAGTAAAG
GCGCAAAACTTTTACGATTCCGATCCTCATATATCAGAGTTAACGCCAAAAAGCTTCGAT
AAAGCGATCCATAACACAAATTACACATCATTAGTGGAATTTTATGCTCCGTGGTGCGGC
CATTGTAAGAAGCTCTCTAGTACGTTCCGCAAGGCAGCAAAAAGATTGGATGGTGTAGTC
CAAGTTGCTGCTGTAAACTGTGACCTTAACAAGAATAAGGCTTTGTGTGCTAAATACGAC
GTAAACGGATTTCCCACGTTAATGGTATTTAGGCCCCCAAAAATTGACCTATCTAAGCCA
ATAGATAACGCCAAAAAAAGTTTCAGCGCTCATGCCAATGAAGTGTACTCAGGTGCAAGA
ACTCTCGCGCCTATTGTTGATTTTTCTCTTTCAAGAATAAGGTCATATGTCAAAAAGTTT
GTCCGTATAGATACACTTGGCTCTTTACTTAGAAAGTCACCCAAACTTTCCGTGGTGTTG
TTTTCCAAACAAGACAAAATTTCACCGGTTTATAAAAGCATTGCCCTTGATTGGTTAGGA
AAGTTCGATTTTTATTCAATTTCAAACAAAAAACTCAAGCAACTAACCGATATGAACCCA
ACATATGAAAAAACTCCTGAGATTTTCAAATATTTGCAGAAGGTCATTCCTGAACAGCGA
CAGAGCGATAAAAGTAAGCTTGTCGTTTTTGATGCAGACAAAGATAAATTTTGGGAGTAT
GAAGGGAACTCAATCAACAAAAATGACATTTCCAAATTTCTGCGGGACACTTTTAGTATT
ACCCCCAATGAGGGTCCTTTTAGTAGACGTTCTGAATATATTGCTTACTTAAAAACTGGC
AAGAAGCCAATTAAAAAGAACCATTCCTCCTCAGGAAACAAGCACGACGAATTGTAG

Further information on MPD1 can be obtained from the URL address http://db.yeastgenome.org/cgi-bin/singlepageformat?sgdid=S000005814.

It will be appreciated that, by “MPD1”, we include fragments or variants thereof having equivalent MPD1-like activity.

MPD2 is another S. cerevisiae helper protein of interest for the present invention. Mpd2p is a member of the protein disulphide isomerase (PDI) family. It exhibits chaperone activity. Its overexpression suppresses the lethality of a PDI1 deletion but does not complement all Pdi1p functions. It undergoes oxidation by Ero1p. A published protein sequence for the protein Mpd2p is as follows:

MKLHGFLFSVLSTCVVILPALAYSEAVTMVKSIEQYFDICNRNDSYTMIK
YYTSWCQHCKTLAPVYEELGELYAKKANKDDTPINFLEVNCEFFGPTLCT
DLPGFPIIELVKPRTKPLVLPKLDWSSMKFHERLWQRIKTWFNNPKYQLD
TSRVVRFEGSRNLKSLSNFIDTVRSKDTEERFIEHIFDDSRNCNEELRSQ
QLLCKAGKEYYSDTLSKLYGDVNGLEKERRRLEALIKQNGDDLSKEVKEK
LKIIRLQLSLLSHIEDQLEDTSSHDEL*

MPD2 is encoded by a non-essential gene comprising an ORF that is 0.834 kbp in size and is located on chromosome XV. A published nucleotide coding sequence of MPD2 is as follows, although it will be appreciated that the sequence can be modified by degenerate substitutions to obtain alternative nucleotide sequences which encode an identical protein product:

ATGAAATTGCACGGCTTTTTATTTTCCGTATTATCAACATGCGTCGTCATTTTACCAGCG
TTGGCCTACAGTGAAGCTGTCACGATGGTCAAGTCGATTGAGCAGTACTTCGATATCTGC
AATAGGAATGATTCTTACACAATGATAAAATACTACACTTCTTGGTGCCAACATTGTAAA
ACTCTGGCCCCAGTATACGAAGAGCTTGGTGAGCTATACGCCAAAAAAGCTAATAAAGAT
GATACCCCAATTAACTTCCTTGAAGTTAACTGTGAATTCTTCGGGCCAACTTTATGTACC
GACTTGCCTGGATTTCCAATAATTGAACTGGTCAAACCTCGTACTAAGCCCTTAGTTCTT
CCGAAGCTCGATTGGTCGTCTATGAAATTTCATGAAAGACTATGGCAAAGAATCAAGACG
TGGTTCAACAATCCTAAGTACCAACTGGATACGTCTAGGGTTGTTCGTTTTGAAGGGAGT
AGGAACCTAAAGAGTTTAAGCAACTTTATCGATACTGTAAGAAGTAAAGATACAGAAGAA
AGATTCATAGAACATATTTTCGATGATTCTAGGAATTGCAATGAAGAATTACGTTCTCAA
CAGCTTCTGTGTAAAGCTGGTAAAGAATACTACTCTGATACTTTATCTAAATTATACGGT
GACGTGAATGGGCTGGAAAAGGAAAGGCGAAGACTAGAAGCTTTAATTAAGCAAAATGGA
GATGACTTGAGTAAAGAAGTTAAAGAAAAACTGAAAATCATTCGTCTACAATTGAGCCTA
TTATCACACATAGAAGACCAGTTAGAAGATACCAGTAGTCATGACGAGCTTTGA

Further information on MPD2 can be obtained from the URL address http://db.yeastgenome.org/cgi-bin/singlepageformat?sgdid=S000005448.

It will be appreciated that, by “MPD2”, we include fragments or variants thereof having equivalent MPD2-like activity.

EPS1 is another S. cerevisiae helper protein of interest for the present invention. Eps1p is a Pdi1p (protein disulphide isomerase)-related protein involved in endoplasmic reticulum retention of resident ER proteins. A published protein sequence for the protein Eps1p is as follows:

MKMNLKRLVVTFFSCITFLLKFTIAAAEPPEGFPEPLNPTNFKEELSKGLHIIDFYSPYC
PHCKHLAPVWMETWEEFKEESKTLNITFSQVNCIESADLCGDENIEYFPEIRLYNPSGYI
KSFTETPRTKESLIAFARRESMDPNNLDTDLDSAKSESQYLEGFDFLELIAGKATRPHLV
SFWPTKDMKNSDDSLEFKNCDKCHEFQRTWKIISRQLAVDDINTGHVNCESNPTICEELG
FGDLVKITNHRADREPKVALVLPNKTSNNLFDYPNGYSAKSDGYVDFARRTFTNSKFPNI
TEGELEKKANRDIDFLQERGRVTNNDIHLVFSYDPETVVIEDFDILEYLIEPLSKIPNIY
LHQIDKNLINLSRNLFGRMYEKINYDASQTQKVFNKEYFTMNTVTQLPTFFMFKDGDPIS
YVFPGYSTTEMRNIDAIMDWVKKYSNPLVTEVDSSNLKKLISFQTKSYSDLAIQLISSTD
HKHIKGSNKLIKNLLLASWEYEHIRMENNFEEINERRARKADGIKKIKEKKAPANKIVDK
MREEIPHMDQKKLLLGYLDISKEKNFFRKYGITGEYKIGDVIIIDKSNNYYYNKDNFGNS
LTSNNPQLLREAFVSLNIPSKALYSSKLKGRLINSPFHNVLSFLDIIHGNGMPGYLIVIV
LFIAILKGPSIYRRYKVRKHYRAKRNAVGILGNMEKKKNQD*

EPS1 is a non-essential gene comprising an ORF that is 2.106 kbp in size and is located on chromosome IX. A published nucleotide coding sequence of EPS1 is as follows, although it will be appreciated that the sequence can be modified by degenerate substitutions to obtain alternative nucleotide sequences which encode an identical protein product:

ATGAAAATGAATCTGAAAAGGCTCGTAGTTACCTTCTTCTCATGCATCACCTTTCTGCTG
AAATTCACTATAGCCGCCGCTGAACCACCAGAGGGCTTTCCAGAGCCCTTAAATCCAACA
AACTTCAAAGAAGAGCTATCTAAGGGGCTGCATATTATTGACTTCTATAGTCCATACTGT
CCGCACTGCAAACATTTAGCACCTGTTTGGATGGAAACATGGGAGGAGTTTAAAGAGGAG
AGCAAAACACTGAACATAACATTTTCACAGGTTAACTGCATCGAGAGCGCCGATTTGTGT
GGAGATGAAAATATTGAATACTTCCCTGAAATTAGACTTTATAACCCCTCAGGATACATC
AAATCGTTCACTGAAACACCGAGGACCAAAGAATCATTAATTGCATTTGCACGCAGGGAG
TCTATGGACCCAAATAACCTCGATACTGATCTGGATTCTGCTAAAAGTGAGAGCCAGTAT
CTCGAAGGCTTTGATTTTCTCGAGCTGATCGCTGGTAAGGCGACTAGGCCACATTTGGTT
TCCTTCTGGCCAACAAAAGATATGAAAAATAGCGATGATTCACTAGAATTCAAAAACTGT
GACAAATGCCATGAATTCCAAAGGACTTGGAAGATCATTTCAAGACAGTTAGCCGTGGAT
GATATCAACACGGGCCACGTTAATTGCGAATCTAATCCAACAATCTGTGAAGAACTGGGC
TTTGGCGACTTGGTGAAAATAACCAACCACAGAGCCGATAGAGAACCCAAGGTAGCATTA
GTCCTACCCAATAAAACCTCAAATAATTTGTTCGACTATCCCAATGGCTACTCAGCGAAG
TCAGATGGCTATGTAGATTTTGCCAGGAGGACTTTTACAAACAGTAAATTTCCCAATATA
ACAGAAGGGGAGCTCGAAAAAAAAGCAAACAGAGACATTGATTTTCTGCAAGAAAGGGGA
CGAGTAACTAATAATGATATCCATTTAGTTTTTTCATATGACCCCGAAACTGTTGTTATT
GAAGATTTTGACATTTTGGAGTATTTAATCGAGCCTTTGTCAAAAATTCCAAACATATAT
TTGCACCAAATTGACAAGAATCTAATAAATTTGTCACGTAATCTTTTTGGAAGAATGTAT
GAAAAGATCAACTACGACGCCAGCCAAACTCAAAAGGTTTTTAACAAAGAATACTTTACT
ATGAATACGGTTACGCAACTCCCAACTTTTTTCATGTTTAAAGATGGTGATCCCATATCC
TATGTTTTCCCCGGATACTCCACAACAGAAATGAGAAATATTGATGCCATTATGGATTGG
GTAAAAAAGTATTCTAATCCCTTAGTTACCGAAGTTGACTCTTCTAATTTGAAAAAATTA
ATTTCCTTCCAAACCAAGAGCTACAGTGATTTAGCAATTCAGTTAATAAGTAGCACTGAC
CACAAACATATCAAAGGAAGCAACAAGCTTATTAAAAACTTGCTCCTCGCAAGTTGGGAG
TATGAACATATTCGGATGGAAAATAACTTCGAAGAAATTAATGAGAGAAGGGCAAGGAAA
GCAGACGGGATCAAGAAAATAAAGGAAAAAAAGGCTCCGGCTAACAAAATTGTTGATAAA
ATGCGTGAAGAGATTCCCCATATGGATCAAAAAAAATTGTTATTAGGATATTTAGATATT
TCAAAGGAGAAGAATTTTTTTAGAAAATATGGTATTACTGGAGAATATAAAATTGGTGAT
GTGATTATCATTGATAAATCAAATAATTACTACTACAATAAAGATAATTTTGGCAACTCC
TTGACTTCTAACAACCCTCAATTGCTGAGAGAAGCATTCGTGTCCTTAAATATTCCATCA
AAAGCTCTATACAGCTCTAAGTTGAAGGGGAGATTGATAAATTCTCCATTCCATAATGTC
CTCAGTTTCCTAGACATAATCCACGGGAACGGCATGCCCGGTTACTTAATTGTTATTGTT
TTGTTTATCGCAATACTCAAAGGTCCATCTATTTACAGAAGATACAAAGTAAGGAAACAC
TATAGGGCGAAAAGGAACGCTGTCGGTATCCTAGGAAATATGGAGAAAAAAAAAAATCAA
GATTAA

Further information on EPS1 can be obtained from the URL address http://db.yeastgenome.org/cgi-bin/singlepageformat?sgdid=S000001267.

It will be appreciated that, by “EPS1”, we include fragments or variants thereof having equivalent EPS1-like activity.

PDI, or a fragment or variant thereof having an equivalent ability to catalyse the formation of disulphide bonds within the lumen of the endoplasmic reticulum (ER), is another S. cerevisiae helper protein of interest for the present invention. By “PDI” we include any protein having the ability to reactivate the ribonuclease activity against RNA of scrambled ribonuclease as described in EP 0 746 611 and Hillson et al, 1984, Methods Enzymol., 107, 281-292.

PDI is an enzyme which typically catalyses thiol:disulphide interchange reactions, and is a major resident protein component of the ER lumen in secretory cells. A body of evidence suggests that it plays a role in secretory protein biosynthesis (Freedman, 1984, Trends Biochem. Sci., 9, 438-41) and this is supported by direct cross-linking studies in situ (Roth and Pierce, 1987, Biochemistry, 26, 4179-82). The finding that microsomal membranes deficient in PDI show a specific defect in cotranslational protein disulphide (Bulleid and Freedman, 1988, Nature, 335, 649-51) implies that the enzyme functions as a catalyst of native disulphide bond formation during the biosynthesis of secretory and cell surface proteins. This role is consistent with what is known of the enzyme's catalytic properties in vitro; it catalyzes thiol: disulphide interchange reactions leading to net protein disulphide formation, breakage or isomerisation, and can typically catalyze protein folding and the formation of native disulphide bonds in a wide variety of reduced, unfolded protein substrates (Freedman et al., 1989, Biochem. Soc. Symp., 55, 167-192). PDI also functions as a chaperone since mutant PDI lacking isomerase activity accelerates protein folding (Hayano et al, 1995, FEBS Letters, 377, 505-511). Recently, sulphydryl oxidation, not disulphide isomerisation was reported to be the principal function of Protein Disulphide Isomerase in S. cerevisiae (Solovyov et al., 2004, J. Biol. Chem., 279 (33) 34095-34100). The DNA and amino acid sequence of the enzyme is known for several species (Scherens et al, 1991, Yeast, 7, 185-193; Farquhar et al, 1991, Gene, 108, 81-89; EP074661; EP0293793; EP0509841) and there is increasing information on the mechanism of action of the enzyme purified to homogeneity from mammalian liver (Creighton et al, 1980, J. Mol. Biol., 142, 43-62; Freedman et al, 1988, Biochem. Soc. Trans., 16, 96-9; Gilbert, 1989, Biochemistry, 28, 7298-7305; Lundstrom and Holmgren, 1990, J. Biol. Chem., 265, 9114-9120; Hawkins and Freedman, 1990, Biochem. J., 275, 335-339). Of the many protein factors currently implicated as mediators of protein folding, assembly and translocation in the cell (Rothman, 1989, Cell, 59, 591-601), PDI has a well-defined catalytic activity.

The deletion or inactivation of the endogenous PDI gene in a host results in the production of an inviable host. In other words, the endogenous PDI gene is an “essential” gene.

PDI is readily isolated from mammalian tissues and the homogeneous enzyme is a homodimer (2×57 kD) with characteristically acidic pI (4.0-4.5) (Hinson et al, 1984, op. cit.). The enzyme has also been purified from wheat and from the alga Chlamydomonas reinhardii (Kaska et al, 1990, Biochem. J., 268, 63-68), rat (Edman et al, 1985, Nature, 317, 267-270), bovine (Yamauchi et al, 1987, Biochem. Biophys. Res. Comm., 146, 1485-1492), human (Pihlajaniemi et al, 1987, EMBO J., 6, 643-9), yeast (Scherens et al, supra; Farquhar et al, op. cit.) and chick (Parkkonen et al, 1988, Biochem. J., 256, 1005-1011). The proteins from these vertebrate species show a high degree of sequence conservation throughout and all show several overall features first noted in the rat PDI sequence (Edman et al., 1985, op. cit.).

Preferred PDI sequences include those from humans and those from yeast species, such as S. cerevisiae.

A yeast protein disulphide isomerase precursor, PDI1, can be found as Genbank accession no. CAA42373 or BAA00723. It has the following sequence of 522 amino acids:

| | |
|---|---|
| 1 | mkfsagavls wsslllassv faqqeavape dsavvklatd sfneyiqshd lvlaeffapw |
| 61 | cghcknmape yvkaaetlve knitlaqidc tenqdlcmeh nipgfpslki fknsdvnnsi |
| 121 | dyegprtaea ivqfmikqsq pavavvadlp aylanetfvt pvivqsgkid adfnatfysm |
| 181 | ankhfndydf vsaenadddf klsiylpsam depvvyngkk adiadadvfe kwlqvealpy |
| 241 | fgeidgsvfa qyvesglplg ylfyndeeel eeykplftel akknrglmnf vsidarkfgr |
| 301 | hagnlnmkeq fplfaihdmt edlkyglpql seeafdelsd kivleskaie slvkdflkgd |
| 361 | aspivksqei fenqdssvfq lvgknhdeiv ndpkkdvlvl yyapwcghck rlaptyqela |
| 421 | dtyanatsdv liakldhten dvrgvviegy ptivlypggk ksesvvyqgs rsldslfdfi |
| 481 | kenghfdvdg kalyeeaqek aaeeadadae ladeedaihd el |

An alternative yeast protein disulphide isomerase sequence can be found as Genbank accession no. CAA38402. It has the following sequence of 530 amino acids

| | |
|---|---|
| 1 | mkfsagavls wsslllassv faqqeavape dsavvklatd sfneyiqshd lvlaeffapw |
| 61 | cghcknmape yvkaaetlve knitlaqidc tenqdlcmeh nipgfpslki fknrdvnnsi |
| 121 | dyegprtaea ivqfmikqsq pavavvadlp aylanetfvt pvivqsgkid adfnatfysm |
| 181 | ankhfndydf vsaenadddf klsiylpsam depvvyngkk adiadadvfe kwlqvealpy |
| 241 | fgeidgsvfa qyvesglplg ylfyndeeel eeykplftel akknrglmnf vsidarkfgr |
| 301 | hagnlnmkeq fplfaihdmt edlkyglpql seeafdelsd kivleskaie slvkdflkgd |
| 361 | aspivksqei fenqdssvfq lvgknhdeiv ndpkkdvlvl yyapwcghck rlaptyqela |
| 421 | dtyanatsdv liakldhten dvrgvviegy ptivlypggk ksesvvyqgs rsldslfdfi |
| 481 | kenghfdvdg kalyeeaqek aaeeaeadae aeadadaela deedaihdel |

The following alignment of these sequences (the sequence of Genbank accession no. CAA42373 or BAA00723 first, the sequence of Genbank accession no. CAA38402 second) shows that the differences between these two sequences are a single amino acid difference at position 114 (highlighted in bold) and that the sequence defined by Genbank accession no. CAA38402 contains the additional amino acids EADAEAEA at positions 506-513.

| | |
|---|---|
| 1 | mkfsagavls wsslllassv faqqeavape dsavvklatd sfneyiqshd lvlaeffapw |
| 1 | mkfsagavls wsslllassv faqqeavape dsavvklatd sfneyiqshd lvlaeffapw |
| 61 | cghcknmape yvkaaetlve knitlaqidc tenqdlcmeh nipgfpslki fknsdvnnsi |
| 61 | cghcknmape yvkaaetlve knitlaqidc tenqdlcmeh nipgfpslki fknrdvnnsi |
| 121 | dyegprtaea ivqfmikqsq pavavvadlp aylanetfvt pvivqsgkid adfnatfysm |
| 181 | dyegprtaea ivqfmikqsq pavavvadlp aylanetfvt pvivqsgkid adfnatfysm |
| 181 | ankhfndydf vsaenadddf klsiylpsam depvvyngkk adiadadvfe kwlqvealpy |
| 181 | ankhfndydf vsaenadddf klsiylpsam depvvyngkk adiadadvfe kwlqvealpy |
| 241 | fgeidgsvfa qyvesglplg ylfyndeeel eeykplftel akknrglmnf vsidarkfgr |
| 241 | fgeidgsvfa qyvesglplg ylfyndeeel eeykplftel akknrglmnf vsidarkfgr |
| 301 | hagnlnmkeq fplfaihdmt edlkyglpql seeafdelsd kivleskaie slvkdflkgd |
| 301 | hagnlnmkeq fplfaihdmt edlkyglpql seeafdelsd kivleskaie slvkdflkgd |
| 361 | aspivksqei fenqdssvfq lvgknhdeiv ndpkkdvlvl yyapwcghck rlaptyqela |
| 361 | aspivksqei fenqdssvfq lvgknhdeiv ndpkkdvlvl yyapwcghck rlaptyqela |
| 421 | dtyanatsdv liakldhten dvrgvviegy ptivlypggk ksesvvyqgs rsldslfdfi |
| 421 | dtyanatsdv liakldhten dvrgvviegy ptivlypggk ksesvvyqgs rsldslfdfi |
| 481 | kenghfdvdg kalyeeaqek aaeea***** ***dadaela deedaihdel |
| 481 | kenghfdvdg kalyeeaqek aaeeaeadae aeadadaela deedaihdel |

It will be appreciated that, by “PDI” and “PDI1”, we include fragments or variants thereof having equivalent PDI-like activity and PDI1-like activity, respectively.

DER1 is another S. cerevisiae helper protein of interest for the present invention. Der1p is an endoplasmic reticulum membrane protein, required for the protein degradation process associated with the ER, and is involved in the retrograde transport of misfolded or unassembled proteins. A published protein sequence for the protein Der1p is as follows:

MDAVILNLLGDIPLVTRLWTIGCLVLSGLTSLRIVDPGKVVYSYDLVF
KKGQYGRLLYSIFDYGAFNWISMINIFVSANHLSTLENSFNLRRKFC
WIIFLLLVILVKMTSIEQPAASLGVLLHENLVYYELKKNGNQMNVRFF
GAIDVSPSIFPIYMNAVMYFVYKRSWLEIAMNFMPGHVIYYMDDIIGKIY
GIDLCKSPYDWFRNTETP*

DER1 is encoded by a non-essential gene comprising an ORF that is 0.636 kbp in size and is located on chromosome II. A published nucleotide coding sequence of DER1 is as follows, although it will be appreciated that the sequence can be modified by degenerate substitutions to obtain alternative nucleotide sequences which encode an identical protein product:

ATGGATGCTGTAATACTGAATCTCTTAGGCGACATTCCTTTGGTCACA
AGATTATGGACAATTGGCTGTCTTGTACTATCAGGTCTCACAAGTCTCC
GGATTGTGGATCCAGGGAAGGTAGTGTACAGTTATGATTTAGTATTCAA
AAAGGGACAATATGGAAGACTACTTTATTCGATATTCGATTACGGCGCA
TTTAATTGGATATCCATGATAAACATCTTTGTCAGCGCTAATCACTTATC
AACTTTGGAAAACTCATTCAATCTGAGAAGAAAATTCTGTTGGATAATAT
TTTTACTGTTGGTGATACTGGTAAAGATGACCAGCATTGAACAACCTGC
AGCATCACTCGGTGTGTTATTGCATGAGAATCTCGTGTACTACGAACTG
AAAAAGAACGGAAACCAAATGAACGTACGATTCTTCGGTGCCATTGAT
GTTTCACCATCTATATTCCCAATCTACATGAATGCAGTAATGTATTTTGT
ATATAAGCGTAGCTGGTTAGAAATTGCCATGAATTTCATGCCAGGTCAC
GTAATTTACTACATGGATGATATAATAGGGAAGATTTATGGCATCGATTT
GTGTAAATCTCCGTACGACTGGTTCCGCAACACTGAAACACCCTAA

Further information on DER1 can be obtained from the URL address http://db.yeastgenome.org/cgi-bin/singlepageformat?sgdid=S000000405.

It will be appreciated that, by “DER1”, we include fragments or variants thereof having equivalent DER1-like activity.

DER3 is another S. cerevisiae helper protein of interest for the present invention and is also known as HRD1. Der3p is a ubiquitin-protein ligase required for endoplasmic reticulum-associated degradation (BRAD) of misfolded proteins. It is genetically linked to the unfolded protein response (UPR) and is thought to be regulated through association with Hrd3p. It contains an H2 ring finger. A published protein sequence for the protein Der3p is as follows:

MVPENRRKQLAIFVVVTYLLTFYCVYSATKTSVSFLQVTLKLNEGF
NLMVLSIFILLNSTLLWQLLTKLLFGELRLIEHEHIFERLPFTIINTLFM
SSLFHERYFFTVAFFGLLLLYLKVFHWILKDRLEALLQSINDSTTMK
TLIFSRFSFNLVLLAVVDYQIITRCISSIYTNQKSDIESTSLYLIQVME
FTMLLIDLLNLFLQTCLNFWEFYRSQQSLSNENNHIVHGDPTDENTVE
SDQSQPVLNDDDDDDDDDRQFTGLEGKFMYEKAIDVFTRFLKTAL
HLSMLIPFRMPMMLLKDVVWDILALYQSGTSLWKIWRNNKQLDDTL
VTVTVEQLQNSANDDNICIICMDELIHSPNQQTWKNKNKKPKRLPCG
HILHLSCLKNWMERSQTCPICRLPVFDEKGNVVQTTFTSNSDITTQTT
VTDSTGIATDQQGFANEVDLLPTRTTSPDIRIVPTQNIDTLAMRTRSTS
TPSPTWYTFPLHKTGDNSVGSSRSAYEFLITNSDEKENGIPVKLTIEN
HEVNSLHGDGGEQIAKKIVIPDKFIQHI*

DER3 is encoded by a non-essential gene comprising an ORF that is 1.656 kbp in size and is located on chromosome XV. A published nucleotide coding sequence of DER3 is as follows, although it will be appreciated that the sequence can be modified by degenerate substitutions to obtain alternative nucleotide sequences which encode an identical protein product:

ATGGTGCCAGAAAATAGAAGGAAACAGTTGGCAATTTTTGTAGTTGTCACATATTTGCTC
ACATTTTATTGCGTGTATTCAGCCACCAAGACAAGCGTTTCCTTTTTGCAAGTAACACTG
AAGCTAAATGAAGGCTTCAATCTAATGGTTTTGTCGATATTCATCTTATTAAATTCTACC
TTACTATGGCAACTCCTAACGAAACTATTATTTGGTGAACTGAGGCTTATTGAGCATGAG
CACATTTTTGAAAGGTTACCATTTACCATTATAAACACCTTGTTTATGTCCTCACTGTTC
CACGAACGGTATTTTTTCACAGTGGCATTTTTTGGACTATTACTACTCTATCTGAAAGTT
TTCCATTGGATTTTAAAGGATAGGCTGGAGGCCTTATTACAGTCAATAAATGATTCCACC
ACAATGAAAACCCTTATCTTTAGTAGATTCTCATTTAACCTCGTACTATTGGCGGTTGTA
GACTACCAGATAATAACACGATGCATCTCCTCCATATATACAAACCAAAAGAGTGATATT
GAATCCACATCCCTTTACCTGATACAAGTAATGGAGTTTACCATGCTTTTGATTGATTTG
CTAAATTTATTCCTACAGACTTGTTTGAATTTCTGGGAATTTTATCGCTCACAACAAAGT
CTGTCTAATGAGAACAACCATATTGTCCATGGCGATCCTACAGATGAAAACACGGTTGAG
TCTGATCAATCTCAGCCAGTGCTGAATGACGACGACGATGACGACGATGATGATAGACAA
TTTACCGGCCTGGAGGGTAAATTCATGTATGAAAAAGCAATTGACGTATTCACAAGATTC
TTAAAAACGGCACTTCATTTGTCTATGCTAATACCATTTAGGATGCCTATGATGCTTTTG
AAAGATGTGGTGTGGGATATCTTGGCACTATATCAAAGTGGCACAAGTTTGTGGAAAATC
TGGAGAAATAACAAACAGCTCGACGACACTCTTGTCACTGTCACCGTAGAACAGCTACAA
AATTCTGCAAATGATGACAATATTTGTATCATTTGTATGGATGAGTTAATACATTCTCCA
AACCAGCAGACGTGGAAGAATAAAAACAAGAAACCCAAAAGGTTACCTTGTGGCCACATA
CTTCATTTGTCGTGTTTAAAGAATTGGATGGAACGTTCTCAGACTTGTCCTATTTGTAGA
TTGCCTGTCTTTGATGAAAAAGGTAATGTTGTGCAAACGACTTTCACTTCCAATAGTGAT
ATCACGACACAGACCACCGTAACAGATAGCACTGGGATAGCGACAGATCAACAAGGTTTC
GCAAACGAAGTAGATCTACTTCCCACAAGAACAACTTCCCCTGATATAAGGATAGTGCCT
ACTCAAAATATAGACACATTAGCAATGAGAACAAGGTCAACCTCTACACCATCTCCTACG
TGGTATACGTTCCCATTACATAAAACTGGTGATAATTCTGTTGGGTCAAGCCGATCAGCC
TACGAATTTTTGATCACAAATTCAGATGAGAAAGAAAATGGTATTCCTGTCAAATTAACA
ATAGAAAATCACGAAGTAAATTCTCTGCATGGAGACGGGGGCGAGCAAATTGCCAAGAAA
ATTGTCATACCAGATAAATTTATCCAGCATATCTAG

Further information on DER3 can be obtained from the URL address http://db.yeastgenome.org/cgi-bin/singlepageformat?sgdid=S000005373.

It will be appreciated that, by “DER3”, we include fragments or variants thereof having equivalent DER3-like activity.

HRD3 is another S. cerevisiae helper protein of interest for the present invention. Hrd3p is a resident protein of the ER membrane that plays a central role in ER-associated protein degradation (ERAD). It forms an HRD complex with Hrd1p and ERAD determinants that engage in lumen to cytosol communication and coordination of ERAD events. A published protein sequence for the protein Hrd3p is as follows:

MITLLLYLCVICNAIVLIRADSIADPWPEARHLLNTIAKSRDPMKEAAMEPNADEFVGFY
VPMDYSPRNEEKNYQSIWQNEITDSQRHIYELLVQSSEQFNNSEATYTLSQIHLWSQYNF
PHNMTLAHKYLEKFNDLTHFTNHSAIFDLAVMYATGGCASGNDQTVIPQDSAKALLYYQR
AAQLGNLKAKQVLAYKYYSGFNVPRNFHKSLVLYRDIAEQLRKSYSRDEWDIVFPYWESY
NVRISDFESGLLGKGLNSVPSSTVRKRTTRPDIGSPFIAQVNGVQMTLQIEPMGRFAFNG
NDGNINGDEDDEDASERRIIRIYYAALNDYKGTYSQSRNCERAKNLLELTYKEFQPHVDN
LDPLQVFYYVRCLQLLGHMYFTGEGSSKPNIHMAEEILTTSLEISRRAQGPIGRACIDLG
LINQYITNNISQAISYYMKAMKTQANNGIVEFQLSKLATSFPEEKIGDPFNLMETAYLNG
FIPAIYEFAVMIESGMNSKSSVENTAYLFKTFVDKNEAIMAPKLRTAFAALINDRSEVAL
WAYSQLAEQGYETAQVSAAYLMYQLPYEFEDPPRTTDQRKTLAISYYTRAFKQGNIDAGV
VAGDIYFQMQNYSKAMALYQGAALKYSIQAIWNLGYMHEHGLGVNRDFHLAKRYYDQVSE
HDHRFYLASKLSVLKLHLKSWLTWITREKVNYWKPSSPLNPNEDTQHSKTSWYKQLTKIL
QRMRHKEDSDKAAEDSHKHRTVVQNGANHRGDDQEEASEILGFQMEDLVTMGCILGIFLL
SILMSTLAARRGWNVRFNGAQLNANGNRQQEQQQQQQAQGPPGWDFNVQIFAI*

HRD3 is encoded by a non-essential gene comprising an ORF that is 2.502 kbp in size and is located on chromosome XII. A published nucleotide coding sequence of HRD3 is as follows, although it will be appreciated that the sequence can be modified by degenerate substitutions to obtain alternative nucleotide sequences which encode an identical protein product:

ATGATAACACTCTTATTATACCTGTGCGTAATATGTAACGCAATAGTGTTAATAAGGGCT
GATTCGATAGCGGACCCTTGGCCTGAAGCGCGACATCTACTAAATACCATAGCTAAGTCC
AGAGACCCAATGAAAGAAGCTGCTATGGAACCCAATGCAGATGAATTTGTTGGATTCTAT
GTACCGATGGATTATTCCCCACGTAATGAGGAAAAAAACTACCAGAGCATTTGGCAAAAC
GAAATCACAGATTCTCAACGTCATATTTATGAATTACTTGTACAATCAAGTGAACAATTC
AACAACTCAGAAGCAACATATACACTTAGCCAGATTCACCTTTGGAGTCAATATAATTTC
CCGCATAATATGACTTTGGCACACAAATACTTAGAAAAATTCAATGATCTAACCCACTTC
ACCAATCATTCGGCCATCTTCGACTTAGCTGTGATGTATGCCACTGGGGGATGTGCTTCT
GGTAATGATCAAACCGTGATCCCTCAGGATTCTGCTAAAGCACTGCTATATTACCAAAGG
GCTGCCCAACTAGGGAATTTAAAGGCTAAGCAAGTGCTAGCTTATAAATACTATTCTGGC
TTCAATGTCCCACGAAATTTTCATAAATCTTTAGTATTGTACAGGGACATTGCTGAACAG
CTGAGAAAGTCGTACTCCAGGGACGAATGGGATATTGTCTTCCCCTATTGGGAAAGTTAC
AACGTGAGAATATCGGATTTTGAGAGTGGCCTATTAGGTAAAGGTTTGAATTCCGTTCCA
TCTTCTACAGTAAGGAAAAGAACTACGAGACCAGATATTGGTTCACCCTTTATTGCGCAA
GTTAACGGTGTACAGATGACCTTGCAAATCGAACCGATGGGTAGGTTCGCTTTCAACGGT
AACGATGGCAACATAAATGGCGACGAAGATGACGAGGATGCCAGTGAAAGACGAATCATT
CGGATATATTATGCAGCTTTGAATGATTATAAAGGAACATATTCACAAAGCAGAAATTGT
GAGCGCGCCAAAAACTTGTTGGAATTAACGTACAAGGAATTTCAGCCTCATGTCGACAAT
TTGGATCCTTTGCAAGTATTTTACTACGTCCGTTGCTTACAATTATTGGGGCACATGTAT
TTCACCGGCGAAGGCTCCTCGAAGCCTAATATTCATATGGCCGAAGAGATCCTGACCACG
TCGCTAGAAATAAGCAGAAGGGCACAGGGACCTATAGGTAGAGCGTGCATAGATCTGGGC
TTAATAAATCAATACATCACAAACAATATTTCTCAAGCAATTTCGTATTATATGAAAGCT
ATGAAAACACAAGCTAACAATGGAATCGTAGAATTCCAATTATCCAAATTGGCCACTTCA
TTCCCTGAAGAAAAAATCGGCGACCCATTTAACTTAATGGAAACTGCCTACTTGAATGGA
TTCATTCCAGCCATATATGAGTTTGCAGTAATGATCGAATCTGGAATGAACAGTAAGAGT
AGTGTGGAAAACACTGCTTACCTGTTCAAAACATTCGTTGACAAAAACGAAGCTATTATG
GCACCTAAACTGAGGACAGCATTTGCCGCATTAATCAACGATCGTTCAGAAGTGGCTTTA
TGGGCTTATTCCCAACTAGCCGAGCAAGGCTACGAGACTGCTCAAGTCTCTGCCGCCTAC
TTAATGTACCAGTTGCCATATGAGTTTGAGGATCCTCCAAGAACCACAGATCAGAGAAAA
ACTTTGGCAATTTCCTACTATACAAGAGCGTTTAAACAGGGAAATATAGATGCTGGTGTT
GTCGCGGGAGATATCTATTTTCAGATGCAGAATTACAGTAAAGCTATGGCTCTTTATCAG
GGTGCAGCTTTGAAGTACTCTATACAGGCTATCTGGAACTTAGGGTACATGCATGAGCAT
GGGCTAGGTGTAAACAGAGATTTCCATCTTGCTAAACGTTACTACGACCAAGTTTCAGAA
CACGATCATAGATTTTACTTGGCTTCCAAATTGAGTGTTTTAAAATTACACCTAAAGTCA
TGGTTGACTTGGATCACCAGAGAAAAAGTAAACTACTGGAAACCTTCCTCGCCACTTAAC
CCTAACGAAGATACTCAGCACTCGAAGACTTCATGGTACAAGCAATTGACGAAGATTCTA
CAAAGAATGAGACATAAGGAGGATAGTGACAAAGCTGCGGAAGATTCTCACAAACACAGA
ACTGTAGTGCAGAATGGAGCTAACCATAGGGGTGACGACCAAGAGGAGGCTTCCGAGATT
TTGGGCTTCCAAATGGAGGATCTTGTTACGATGGGATGTATCTTGGGGATATTCCTATTA
AGTATATTAATGAGTACACTGGCGGCCCGTAGAGGCTGGAATGTCCGTTTCAATGGAGCA
CAATTAAATGCAAATGGTAACCGGCAGCAAGAGCAACAACAACAACAACAAGCACAAGGT
CCCCCGGGCTGGGACTTCAATGTTCAGATATTCGCCATATGA

Further information on HRD3 can be obtained from the URL address http://db.yeastgenome.org/cgi-bin/singlepageformat?sgdid=S000004197.

It will be appreciated that, by “HRD3”, we include fragments or variants thereof having equivalent HRD3-like activity.

UBC7 is another S. cerevisiae helper protein of interest for the present invention and is also known as QRI8. Ubc7p is a ubiquitin conjugating enzyme, involved in the ER-associated protein degradation pathway. It requires Cue1p for recruitment to the ER membrane and is proposed to be involved in chromatin assembly. A published protein sequence for the protein Ubc7p is as follows:

MSKTAQKRLLKELQQLIKDSPPGIVAGPKSENNIFIWDCLIQGPPDTP
YADGVFNAKLEFPKDYPLSPPKLTFTPSILHPNIYPNGEVCISILHSPG
DDPNMYELAEERWSPVQSVEKILLSVMSMLSEPNIESGANIDACILW
RDNRPEFERQVKLSILKSLGF*

UBC7 is encoded by a non-essential gene comprising an ORF that is 0.498 kbp in size and is located on chromosome XIII. A published nucleotide coding sequence of UBC7 is as follows, although it will be appreciated that the sequence can be modified by degenerate substitutions to obtain alternative nucleotide sequences which encode an identical protein product:

ATGTCGAAAACCGCTCAGAAACGTCTCCTCAAGGAGCTTCAACAGTTA
ATTAAAGATTCTCCACCTGGTATAGTGGCTGGTCCCAAATCGGAGAATA
ACATATTCATTTGGGACTGCCTAATTCAAGGGCCTCCAGATACGCCATA
CGCTGATGGTGTTTTTAATGCTAAGCTAGAGTTTCCTAAAGACTATCCGT
TATCTCCACCTAAACTTACTTTCACACCCAGCATACTACATCCAAATATT
TATCCAAATGGGGAAGTGTGCATATCCATTCTACACTCCCCTGGTGATG
ATCCTAACATGTACGAATTAGCGGAAGAAAGATGGTCGCCAGTGCAAA
GTGTAGAAAAAATTCTATTAAGTGTTATGAGCATGTTGAGTGAGCCCAAT
ATCGAAAGTGGTGCCAACATTGATGCTTGCATCTTGTGGAGAGATAATA
GACCTGAATTTGAGAGACAGGTAAAGTTATCCATTTTGAAATCATTAGGA
TTCTGA

Further information on UBC7 can be obtained from the URL address http://db.yeastgenome.org/cgi-bin/singlepageformat?sgdid=S000004624.

It will be appreciated that, by “UBC7”, we include fragments or variants thereof having equivalent UBC7-like activity.

DOA4 is another S. cerevisiae helper protein of interest for the present invention and is also known as DOS1, MUT4, NPI2, SSV7, and UBP4. Doa4p is a ubiquitin hydrolase, required for recycling ubiquitin from proteasome-bound ubiquitinated intermediates, which acts at the late endosome/prevacuolar compartment to recover ubiquitin from ubiquitinated membrane proteins en route to the vacuole. A published protein sequence for the protein Doa4p is as follows:

MEQNIISTIRDECIRHRSKYLTIAQLTAIAEAKINEFIITGKAKDQDLSSLLDKCIDILS
IYKKNSKDIKNIISCKNKGAMISSNSVMIIQLNYVYYKVIHIIVTTNIPHLSEFAKIKLH
KSTSDEGNGNNNNNEFQLMNIYNTLLETLLKDENIAKIKSFIKSSIKQTKLNHEQEECNL
MRTGSYITSNQLNSLISSSANSASSQMEILLIDIRSRLEFNKSHIDTKNIICLEPISFKM
SYSDHDLEKKSLITSPNSEIKMFQSRNLFKFIILYTDANEYNVKQQSVLLDILVNHSFEK
PISDDFTKIFILESGFPGWLKSNYGRQVSSSFPSNNNIKDDSVYINGNTSGLSLQHLPKM
SPSIRHSMDDSMKEMLVAPTPLNHLQQQQQQQSDNDHVLKRSSSFKKLFSNYTSPNPKNS
NSNLYSISSLSISSSPSPLPLHSPDPVKGNSLPINYPETPHLWKNSETDFMTNQREQLNH
NSFAHIAPINTKAITSPSRTATPKLQRFPQTISMNLNMNSNGHSSATSTIQPSCLSLSNN
DSLDHTDVTPTSSHNYDLDFAVGLENLGNSCYMNCIIQCILGTHELTQIFLDDSYAKHIN
INSKLGSKGILAKYFARLVHMMYKEQVDGSKKISISPIKFKLACGSVNSLFKTASQQDCQ
EFCQFLLDGLHEDLNQCGSNPPLKELSQEAEARREKLSLRIASSIEWERFLTTDFSVIVD
LFQGQYASRLKCKVCSHTSTTYQPFTVLSIPIPKKNSRNNITIEDCFREFTKCENLEVDE
QWLCPHCEKRQPSTKQLTITRLPRNLIVHLKRFDNLLNKNNDFVIYPFLLDLTPFWANDF
DGVFPPGVNDDELPIRGQIPPFKYELYGVACHFGTLYGGHYTAYVKKGLKKGWLYFDDTK
YKPVKNKADAINSNAYVLFYHRVYGV*

DOA4 is encoded by a non-essential gene comprising an ORF that is 2.781 kbp in size and is located on chromosome IV. A published nucleotide coding sequence of DOA4 is as follows, although it will be appreciated that the sequence can be modified by degenerate substitutions to obtain alternative nucleotide sequences which encode an identical protein product:

ATGGAGCAGAATATTATTAGTACCATAAGGGATGAGTGTATTCGTCACCGGTCGAAGTAC
CTTACGATAGCACAACTAACCGCTATTGCAGAGGCTAAAATTAACGAATTCATCATAACT
GGTAAGGCAAAAGATCAAGATTTGAGCAGTCTTCTAGATAAATGCATCGATATTTTATCT
ATTTACAAGAAGAACTCGAAAGATATCAAAAATATTATATCGTGCAAAAATAAGGGTGCA
ATGATTAGTTCAAATTCCGTAATGATTATTCAATTAAATTATGTTTACTACAAGGTAATT
CACATTATTGTAACAACCAATATTCCTCATTTAAGTGAATTCGCCAAGATTAAATTACAT
AAGAGCACGAGTGATGAGGGCAACGGTAATAACAACAATAATGAATTTCAACTCATGAAC
ATTTACAACACTTTGCTGGAAACCTTATTAAAAGATGAAAACATTGCAAAAATAAAAAGT
TTCATTAAGTCTTCCATAAAACAAACAAAATTGAACCATGAGCAAGAAGAATGTAACCTG
ATGAGAACGGGTTCCTATATCACTTCCAATCAATTAAACTCCCTAATAAGTTCATCAGCA
AATTCTGCTTCCTCCCAAATGGAGATACTACTGATAGATATACGATCAAGGTTGGAATTC
AACAAGTCACATATTGATACAAAAAATATTATATGCCTGGAGCCTATTTCTTTTAAAATG
TCATATTCAGATCATGATTTGGAGAAAAAATCATTAATTACTTCTCCTAATAGTGAGATT
AAAATGTTTCAAAGTAGAAATCTTTTCAAGTTTATCATTCTCTATACAGACGCAAACGAA
TACAATGTTAAACAGCAGTCTGTCCTGTTGGACATTCTGGTGAATCATTCCTTTGAAAAA
CCAATATCCGATGACTTTACCAAAATTTTCATTCTGGAATCTGGTTTTCCAGGTTGGCTT
AAGTCAAATTATGGGAGGCAAGTATCATCATCTTTTCCATCAAATAACAATATTAAAGAT
GATAGTGTTTATATTAATGGTAACACTTCTGGCCTAAGTTTACAACATTTACCTAAGATG
TCTCCCAGTATAAGACATTCAATGGACGACTCTATGAAAGAAATGCTAGTTGCGCCTACT
CCATTAAATCATCTTCAACAACAGCAACAACAGCAATCAGACAATGATCATGTGCTAAAA
AGATCTTCAAGTTTCAAAAAATTATTCTCAAATTATACGTCTCCTAATCCGAAGAATTCA
AATTCAAACTTATATTCTATATCTTCGTTGTCCATATCTAGTTCACCATCGCCTTTACCT
CTACATTCGCCTGACCCAGTTAAGGGCAATTCATTGCCAATCAATTATCCGGAAACGCCA
CATCTTTGGAAAAACAGTGAGACAGATTTTATGACAAATCAAAGAGAACAGTTGAATCAC
AACTCTTTTGCTCACATAGCTCCTATCAACACGAAGGCCATCACTTCTCCATCAAGAACT
GCCACACCGAAGTTACAACGCTTCCCGCAAACAATTAGTATGAACCTTAATATGAACTCC
AATGGACACAGTTCTGCCACCTCTACCATTCAACCTTCGTGTCTATCCTTGTCTAATAAT
GACTCTTTAGATCATACAGATGTTACACCAACTTCTTCTCATAATTATGACCTTGATTTC
GCGGTTGGTTTGGAAAATCTAGGAAATTCGTGTTACATGAACTGTATCATTCAGTGTATC
TTAGGTACACACGAATTAACCCAAATCTTTTTGGACGATTCATATGCTAAACACATCAAT
ATTAATAGTAAGTTGGGATCGAAAGGTATTCTGGCAAAATATTTTGCAAGGTTGGTTCAT
ATGATGTATAAGGAACAGGTTGATGGTTCAAAGAAAATTTCCATATCACCGATAAAATTT
AAATTGGCATGTGGATCTGTTAACTCATTATTTAAGACTGCATCCCAACAGGACTGCCAA
GAGTTTTGCCAATTCCTTCTAGATGGTCTTCATGAAGACTTGAACCAATGCGGTTCAAAC
CCACCTTTGAAGGAGCTTTCTCAAGAAGCTGAGGCGAGAAGAGAAAAACTGTCTTTGCGA
ATTGCCTCGTCAATTGAGTGGGAACGATTCTTGACTACTGATTTCAGTGTTATTGTCGAC
TTATTTCAGGGACAATACGCCTCACGACTAAAATGTAAAGTCTGTAGTCATACCTCGACA
ACATACCAACCTTTTACGGTGCTGTCAATCCCTATTCCTAAAAAAAATTCCCGAAATAAT
ATTACCATTGAAGATTGTTTCAGAGAGTTCACCAAATGTGAGAACTTGGAAGTGGATGAG
CAATGGTTGTGCCCACATTGTGAAAAAAGGCAGCCCTCCACGAAACAATTGACAATAACG
AGATTACCGAGGAATCTGATAGTCCATTTAAAGAGATTTGATAATTTATTAAACAAAAAT
AATGACTTCGTCATATACCCTTTTTTGTTGGACTTGACTCCATTTTGGGCCAATGATTTT
GACGGGGTTTTTCCTCCAGGTGTTAATGACGATGAACTACCAATAAGGGGACAAATACCA
CCTTTTAAGTATGAATTATATGGTGTAGCATGCCACTTTGGTACTTTGTATGGTGGTCAT
TATACAGCCTATGTGAAAAAGGGATTAAAGAAGGGATGGCTATATTTTGATGATACCAAA
TATAAACCTGTCAAAAACAAAGCCGATGCAATTAACTCTAATGCATACGTTTTGTTTTAT
CACCGCGTCTACGGTGTTTGA

Further information on DOA4 can be obtained from the URL address http://db.yeastgenome.org/cgi-bin/singlepageformat?sgdid=S000002476.

It will be appreciated that, by “DOA4”, we include fragments or variants thereof having equivalent DOA4-like activity.

HAC1 is another S. cerevisiae helper protein of interest for the present invention, and is also known as ERN4 and IRE15. Hac1p, is a bZIP transcription factor (ATF/CREB1 homolog) that regulates the unfolded protein response, via UPRE binding, and membrane biogenesis. ER stress-induced splicing pathway utilising Ire1p, Trl1p and Ada5p facilitates efficient Hac1p synthesis. A published protein sequence for the protein Hac1p is as follows:

MEMTDFELTSNSQSNLAIPTNFKSTLPPRKRAKTKEEKEQRRIERILRNR
RAAHQSREKKRLHLQYLERKCSLLENLLNSVNLEKLADHEDALTCSHD
AFVASLDEYRDFQSTRGASLDTRASSHSSSDTFTPSPLNCTMEPATLS
PKSMRDSASDQETSWELQMFKTENVPESTTLPAVDNNNLFDAVASPL
ADPLCDDIAGNSLPFDNSIDLDNWRNPEAQSGLNSFELNDFFITS*

HAC1 is encoded by a non-essential gene that is located on chromosome VI. A published nucleotide coding sequence of HAC1, that has been processed to remove introns, is 0.717 kbp in size and is as follows (although it will be appreciated that the sequence can be modified by degenerate substitutions to obtain alternative nucleotide sequences which encode an identical protein product):

ATGGAAATGACTGATTTTGAACTAACTAGTAATTCGCAATCGAACTTGGCTATCCCTACC
AACTTCAAGTCGACTCTGCCTCCAAGGAAAAGAGCCAAGACAAAAGAGGAAAAGGAACAG
CGAAGGATCGAGCGTATTTTGAGAAACAGAAGAGCTGCTCACCAGAGCAGAGAGAAAAAA
AGACTACATCTGCAGTATCTCGAGAGAAAATGTTCTCTTTTGGAAAATTTACTGAACAGC
GTCAACCTTGAAAAACTGGCTGACCACGAAGACGCGTTGACTTGCAGCCACGACGCTTTT
GTTGCTTCTCTTGACGAGTACAGGGATTTCCAGAGCACGAGGGGCGCTTCACTGGACACC
AGGGCCAGTTCGCACTCGTCGTCTGATACGTTCACACCTTCACCTCTGAACTGTACAATG
GAGCCTGCGACTTTGTCGCCCAAGAGTATGCGCGATTCCGCGTCGGACCAAGAGACTTCA
TGGGAGCTGCAGATGTTTAAGACGGAAAATGTACCAGAGTCGACGACGCTACCTGCCGTA
GACAACAACAATTTGTTTGATGCGGTGGCCTCGCCGTTGGCAGACCCACTCTGCGACGAT
ATAGCGGGAAACAGTCTACCCTTTGACAATTCAATTGATCTTGACAATTGGCGTAATCCA
GAAGCGCAGTCAGGTTTGAATTCATTTGAATTGAATGATTTCTTCATCACTTCATGA

Further information on HAC1 can be obtained from the URL address http://db.yeastgenome.org/cgi-bin/singlepageformat?sgdid=S000001863.

It will be appreciated that, by “HAC1”, we include fragments or variants thereof having equivalent HAC1-like activity.

SEC63 is another S. cerevisiae helper protein of interest for the present invention. It is also known as PTL1. It is an essential subunit of the Sec63 complex (Sec63p, Sec62p, Sec66p and Sec72p); with Sec61 complex, Kar2p/BiP and Lhs1p it forms a channel competent for SRP-dependent and post-translational SRP-independent protein targeting and import into the ER. A published protein sequence for the protein Sec63p is as follows:

MPTNYEYDEASETWPSFILTGLLMVVGPMTLLQIYQIFFGANAEDGNSGKSKEFNEEVFK
NLNEEYTSDEIKQFRRKFDKNSNKKSKIWSRRNIIIIVGWILVAILLQRINSNDAIKDAA
TKLFDPYEILGISTSASDRDIKSAYRKLSVKFHPDKLAKGLTPDEKSVMEETYVQITKAY
ESLTDELVRQNYLKYGHPDGPQSTSHGIALPRFLVDGSASPLLVVCYVALLGLILPYFVS
RWWARTQSYTKKGIHNVTASNFVSNLVNYKPSEIVTTDLILHWLSFAHEFKQFFPDLQPT
DFEKLLQDHINRRDSGKLNNAKFRIVAKCHSLLHGLLDIACGFRNLDIALGAINTFKCIV
QAVPLTPNCQILQLPNVDKEHFITKTGDIHTLGKLFTLEDAKIGEVLGIKDQAKLNETLR
VASHIPNLKIIKADFLVPGENQVTPSSTPYISLKVLVRSAKQPLIPTSLIPEENLTEPQD
FESQRDPFAMMSKQPLVPYSFAPFFPTKRRGSWCCLVSSQKDGKILQTPIIIEKLSYKNL
NDDKDFFDKRIKMDLTKHEKFDINDWEIGTIKIPLGQPAPETVGDFFFRVIVKSTDYFTT
DLDITMNMKVRDSPAVEQVEVYSEEDDEYSTDDDETESDDESDASDYTDIDTDTEAEDDE
SPE*

SEC63 is encoded by an essential gene comprising an ORF that is 1.192 kbp in size and is located on chromosome XV. A published nucleotide coding sequence of SEC63 is as follows, although it will be appreciated that the sequence can be modified by degenerate substitutions to obtain alternative nucleotide sequences which encode an identical protein product:

ATGCCTACAAATTACGAGTATGATGAGGCTAGTGAGACGTGGCCGTCCTTCATTTTAACG
GGGCTCTTGATGGTCGTCGGGCCTATGACACTGCTTCAAATATACCAAATTTTTTTTGGG
GCCAATGCTGAAGATGGGAATTCAGGGAAGAGTAAGGAGTTTAATGAGGAAGTTTTCAAG
AACTTGAATGAAGAATACACCAGTGATGAAATCAAACAATTTAGAAGGAAGTTTGATAAA
AATAGTAATAAGAAGTCCAAAATATGGAGCAGGAGAAATATTATAATTATTGTGGGTTGG
ATCTTAGTTGCAATTCTTCTGCAAAGGATTAATAGTAATGACGCGATTAAAGACGCTGCT
ACAAAATTATTTGATCCTTATGAAATCCTTGGTATCTCTACTAGTGCTTCCGATAGAGAC
ATCAAATCTGCTTATAGAAAATTATCTGTTAAATTTCATCCAGATAAATTAGCAAAGGGC
CTAACACCTGATGAGAAAAGTGTGATGGAAGAAACTTATGTTCAGATTACGAAGGCTTAC
GAATCCCTTACTGACGAATTGGTTAGGCAAAACTATTTGAAATACGGTCATCCAGATGGC
CCACAATCTACTTCACATGGTATCGCTCTACCAAGATTTTTGGTAGATGGAAGTGCATCT
CCATTATTAGTGGTTTGTTATGTTGCGCTACTAGGTTTAATCTTGCCATATTTTGTTAGT
AGATGGTGGGCAAGAACACAATCGTATACTAAGAAGGGAATACATAATGTGACGGCTTCT
AATTTTGTTAGTAACTTAGTCAATTACAAGCCATCTGAGATTGTCACCACAGATTTGATC
TTACACTGGTTATCATTTGCTCATGAATTTAAACAATTCTTCCCGGATTTGCAACCAACG
GATTTTGAAAAACTTTTGCAAGATCATATTAACCGCAGAGATAGTGGTAAACTTAACAAT
GCGAAATTTAGAATAGTGGCCAAATGTCACTCTTTGTTACACGGTTTATTGGATATTGCT
TGTGGATTCAGAAATTTAGATATTGCATTGGGTGCAATCAATACTTTCAAGTGTATTGTT
CAGGCTGTACCATTAACACCAAACTGTCAAATCCTTCAATTGCCGAACGTAGATAAAGAG
CACTTTATTACCAAAACCGGAGATATTCATACATTAGGTAAATTGTTTACTTTAGAAGAT
GCCAAGATTGGTGAGGTTCTTGGAATAAAGGATCAAGCAAAGTTAAACGAAACTTTGAGA
GTTGCATCGCATATTCCAAATCTAAAGATCATCAAGGCAGACTTCCTTGTCCCAGGTGAG
AACCAAGTAACACCATCATCTACCCCATACATTTCTTTGAAAGTACTGGTTCGTTCTGCT
AAACAGCCATTGATACCAACTAGCTTAATTCCTGAAGAAAATTTAACAGAACCTCAAGAT
TTTGAATCTCAAAGAGATCCATTTGCTATGATGAGTAAACAGCCACTCGTCCCATATTCC
TTTGCACCATTTTTCCCTACAAAGAGACGTGGGAGTTGGTGCTGTCTGGTAAGTTCTCAA
AAAGATGGTAAAATACTTCAAACGCCAATTATCATTGAAAAGCTATCTTACAAGAACTTG
AACGATGACAAAGATTTCTTTGATAAGAGGATAAAAATGGATTTAACCAAACACGAAAAA
TTCGATATAAATGATTGGGAAATCGGGACCATAAAAATTCCATTAGGTCAGCCTGCACCT
GAAACTGTTGGTGATTTCTTTTTTAGAGTAATCGTTAAATCCACAGATTATTTCACTACA
GATTTGGATATTACCATGAATATGAAAGTTCGTGATTCTCCTGCAGTGGAACAAGTAGAG
GTGTATTCTGAGGAGGATGATGAGTACTCTACTGATGACGACGAAACCGAAAGTGATGAT
GAAAGTGATGCTAGCGATTATACTGATATCGATACGGATACAGAAGCTGAAGATGATGAA
TCACCAGAATAG

Further information on SEC63 can be obtained from the URL address http://db.yeastgenome.org/cgi-bin/singlepageformat?sgdid=S000005780

It will be appreciated that, by “SEC63”, we include fragments or variants thereof having equivalent SEC63-like activity.

YDJ1 is another S. cerevisiae helper protein of interest for the present invention. It is also known as MASS and HSP40. It is a protein chaperone involved in regulation of the HSP90 and HSP70 functions; involved in protein translocation across membranes; member of the DnaJ family, and is located in the cytoplasm. A published protein sequence for the protein Ydj1p is as follows:

MVKETKFYDILGVPVTATDVEIKKAYRKCALKYHPDKNPSEEAAEKFK
EASAAYEILSDPEKRDIYDQFGEDGLSGAGGAGGFPGGGFGFGDDIF
SQFFGAGGAQRPRGPQRGKDIKHEISASLEELYKGRTAKLALNKQILC
KECEGRGGKKGAVKKCTSCNGQGIKFVTRQMGPMIQRFQTECDVCH
GTGDIIDPKDRCKSCNGKKVENERKILEVHVEPGMKDGQRIVFKGEAD
QAPDVIPGDVVFIVSERPHKSFKRDGDDLVYEAEIDLLTAIAGGEFALE
HVSGDWLKVGIVPGEVIAPGMRKVIEGKGMPIPKYGGYGNLIIKFTIKFP
ENHFTSEENLKKLEEILPPRIVPAIPKKATVDECVLADFDPAKYNRTRA
SRGGANYDSDEEEQGGEGVQCASQ*

YDJ1 is encoded by a non-essential gene comprising an ORF that is 1.230 kbp in size and is located on chromosome XIV. A published nucleotide coding sequence of YDJ1 is as follows, although it will be appreciated that the sequence can be modified by degenerate substitutions to obtain alternative nucleotide sequences which encode an identical protein product:

ATGGTTAAAGAAACTAAGTTTTACGATATTCTAGGTGTTCCAGTAACTGCCACTGATGTC
GAAATTAAGAAAGCTTATAGAAAATGCGCCTTAAAATACCATCCAGATAAGAATCCAAGT
GAGGAAGCTGCAGAAAAGTTCAAAGAAGCTTCAGCAGCCTATGAAATTTTATCAGATCCT
GAAAAGAGAGATATATATGACCAATTTGGTGAAGATGGTCTAAGTGGTGCTGGTGGCGCT
GGCGGATTCCCAGGTGGTGGATTCGGTTTTGGTGACGATATCTTTTCCCAATTCTTTGGT
GCTGGTGGCGCACAAAGACCAAGAGGTCCCCAAAGAGGTAAAGATATCAAGCATGAAATT
TCTGCCTCACTTGAAGAATTATATAAGGGTAGGACAGCTAAGTTAGCCCTTAACAAACAG
ATCCTATGTAAAGAATGTGAAGGTCGTGGTGGTAAGAAAGGCGCCGTCAAGAAGTGTACC
AGCTGTAATGGTCAAGGTATTAAATTTGTAACAAGACAAATGGGTCCAATGATCCAAAGA
TTCCAAACAGAGTGTGATGTCTGTCACGGTACTGGTGATATCATTGATCCTAAGGATCGT
TGTAAATCTTGTAACGGTAAGAAAGTTGAAAACGAAAGGAAGATCCTAGAAGTCCATGTC
GAACCAGGTATGAAAGATGGTCAAAGAATCGTTTTCAAAGGTGAAGCTGACCAAGCCCCA
GATGTCATTCCAGGTGATGTTGTCTTCATAGTTTCTGAGAGACCACACAAGAGCTTCAAG
AGAGATGGTGATGATTTAGTATATGAGGCTGAAATTGATCTATTGACTGCTATCGCTGGT
GGTGAATTTGCATTGGAACATGTTTCTGGTGATTGGTTAAAGGTCGGTATTGTTCCAGGT
GAAGTTATTGCCCCAGGTATGCGTAAGGTCATCGAAGGTAAAGGTATGCCAATTCCAAAA
TACGGTGGCTATGGTAATTTAATCATCAAATTTACTATCAAGTTCCCAGAAAACCATTTC
ACATCAGAAGAAAACTTGAAGAAGTTAGAAGAAATTTTGCCTCCAAGAATTGTCCCAGCC
ATTCCAAAGAAAGCTACTGTGGACGAATGTGTACTCGCAGACTTTGACCCAGCCAAATAC
AACAGAACACGGGCCTCCAGGGGTGGTGCAAACTATGATTCCGATGAAGAAGAACAAGGT
GGCGAAGGTGTTCAATGTGCATCTCAATGA

Further information on YDJ1 can be obtained from the URL address http://db.yeastgenome.org/cgi-bin/singlepageformat?sgdid=S000005008

It will be appreciated that, by “YDJ1”, we include fragments or variants thereof having equivalent YDJ1-like activity.

XDJ1 is another S. cerevisiae helper protein of interest for the present invention. It is a putative chaperone, a homolog of E. coli DnaJ, and is closely related to Ydj1p. A published protein sequence for the protein Xdj1p is as follows:

MSGSDRGDRLYDVLGVTRDATVQEIKTAYRKLALKHHPDKYVDQDSKEVN
EIKFKEITAAYEILSDPEKKSHYDLYGDDNGAASSGGANGFGDEDFMNFF
NNFFNNGSHDGNNFPGEYDAYEEGNSTSSKDIDIDISLTLKDLYMGKKLK
FDLKRQVICIKCHGSGWKPKRKIHVTHDVECESCAGKGSKERLKRFGPGL
VASQWVVCEKCNGKGKYTKRPKNPKNFCPDCAGLGLLSKKEIITVNVAPG
HHFNDVITVKGMADEEIDKTTCGDLKFHLTEKQENLEQKQIFLKNFDDGA
GEDLYTSITISLSEALTGFEKFLTKTFDDRLLTLSVKPGRVVRPGDTIKI
ANEGWPILDNPHGRCGDLYVFVHIEFPPDNWFNEKSELLAIKTNLPSSSS
CASHATVNTEDDSNLTNNETISNFRIIHTDDLPEGIRPFKPEAQDSAHQK
ARSSYCCIQ*

XDJ1 is encoded by a non-essential gene comprising an ORF that is 1.380 kbp in size and is located on chromosome XII. A published nucleotide coding sequence of XDJ1 is as follows, although it will be appreciated that the sequence can be modified by degenerate substitutions to obtain alternative nucleotide sequences which encode an identical protein product:

ATGAGTGGCAGTGATAGAGGAGACCGGTTATACGATGTGTTGGGGGTGACGAGAGATGCG
ACCGTGCAAGAGATTAAAACTGCTTACAGAAAGCTTGCCCTGAAACATCATCCGGACAAG
TATGTGGATCAAGACTCAAAGGAGGTAAATGAAATCAAATTCAAAGAGATCACTGCCGCT
TACGAGATCTTGAGCGATCCGGAGAAGAAATCACATTACGACTTGTATGGTGATGATAAT
GGTGCCGCTAGCAGCGGTGGCGCTAATGGCTTTGGAGATGAAGATTTTATGAACTTCTTT
AACAATTTCTTCAATAATGGAAGTCACGATGGAAATAATTTCCCTGGCGAGTATGATGCG
TACGAAGAGGGCAACTCTACAAGCTCTAAGGATATCGATATCGATATATCTCTTACTTTG
AAGGATTTGTACATGGGCAAGAAGCTGAAGTTTGATTTAAAGAGACAGGTCATCTGTATA
AAGTGCCACGGTTCTGGCTGGAAACCAAAGAGGAAAATTCACGTTACACACGATGTGGAA
TGTGAATCATGCGCTGGAAAGGGTTCAAAGGAACGTCTGAAGAGGTTTGGTCCCGGTTTG
GTAGCTTCGCAATGGGTGGTCTGTGAGAAATGTAATGGTAAGGGGAAGTACACTAAAAGA
CCCAAGAATCCAAAGAACTTTTGCCCCGATTGCGCAGGCTTGGGGCTCCTGTCAAAGAAG
GAAATCATCACAGTGAACGTGGCTCCGGGACACCACTTTAACGACGTAATTACAGTCAAG
GGGATGGCGGACGAGGAAATCGATAAGACCACATGTGGTGATTTAAAGTTCCATCTCACT
GAAAAACAAGAAAACTTGGAGCAGAAGCAAATCTTTTTGAAGAACTTTGACGACGGCGCC
GGGGAAGATTTGTATACAAGCATTACCATATCGTTAAGCGAGGCCTTGACGGGATTTGAG
AAATTTTTGACAAAAACCTTCGACGACAGGTTACTAACATTGAGCGTTAAACCTGGCAGA
GTAGTAAGACCTGGTGACACCATCAAAATCGCCAATGAAGGTTGGCCCATTTTAGATAAC
CCTCATGGCCGGTGCGGCGATCTGTATGTTTTCGTTCATATTGAATTTCCACCAGATAAC
TGGTTCAATGAAAAATCAGAACTACTAGCAATAAAAACGAATCTGCCGTCATCTTCATCT
TGTGCCTCACATGCGACTGTAAATACTGAAGATGACAGCAACCTGACTAACAACGAAACT
ATATCAAATTTCCGGATCATTCACACGGACGATCTTCCAGAAGGGATAAGGCCGTTCAAG
CCAGAAGCACAGGATTCAGCGCATCAGAAAGCAAGAAGTTCGTACTGCTGTATCCAATGA

Further information on XDJ1 can be obtained from the URL address http://db.yeastgenome.org/cgi-bin/singlepageformat?sgdid=S000004080

It will be appreciated that, by “XDJ1”, we include fragments or variants thereof having equivalent XDJ1-like activity.

APJ1 is another S. cerevisiae helper protein of interest for the present invention. It is a putative chaperone of the HSP40 (DnaJ) family; over expression of which interferes with propagation of the [Psi+] prion. A published protein sequence for the protein Apj1p is as follows:

MQQNTSLYDSLNVTAAASTSEIKKAYRNAALKYHPDKNNHTEESKRKFQEICQAYEILKD
NRLRALYDQYGTTDEVLIQEQQAQAQRQQAGPFSSSSNFDTEAMSFPDLSPGDLFAQFFN
SSATPSSNGSKSSFNFSFNNSSTPSFSFVNGSGVNNLYSSSAKYNSNDEDHHLDRGPDIK
HNLKCTLKELYMGKTAKLGLNRTRICSVCDGHGGLKKCTCKTCKGQGIQTQTRRMGPLVQ
SWSQTCADCGGAGVFVKNKDICQQCQGLGFIKERKILQVTVQPGSCHNQLIVLTGEGDEV
ISTKGGGHEKVIPGDVVITILRLKDPNFQVINYSNLICKKCKIDFMTSLCGGVVYIEGHP
SGKLIKLDIIPGEILKPGCFKTVEDMGMPKFINGVRSGFGHLYVKFDVTYPERLEPENAK
KIQNILANDKYIKAERSTMETADSDCYCDLEKSYDSVEEHVLSSFEAPNLNNEVIEDDDL
GDLINERDSRKRNNRRFDESNINNNNETKRNKYSSPVSGFYDHDINGY*

APJ1 is encoded by a non-essential gene comprising an ORF that is 1.587 kbp in size and is located on chromosome XIV. A published nucleotide coding sequence of APJ1 is as follows, although it will be appreciated that the sequence can be modified by degenerate substitutions to obtain alternative nucleotide sequences which encode an identical protein product:

ATGCAACAAAACACGTCTTTATATGACTCTTTGAACGTTACTGCCGCTGCATCCACATCT
GAGATTAAGAAAGCTTACAGGAACGCTGCATTAAAATATCATCCTGATAAAAACAATCAT
ACAGAAGAATCCAAGCGAAAGTTTCAAGAGATATGCCAGGCATACGAAATACTTAAAGAC
AATCGTTTAAGAGCTTTGTATGACCAGTACGGTACCACAGATGAAGTCCTGATTCAAGAG
CAGCAGGCGCAGGCGCAACGCCAACAAGCCGGGCCGTTCAGTTCATCCTCAAATTTCGAT
ACGGAAGCAATGTCATTCCCGGATCTATCTCCAGGTGATCTTTTCGCGCAGTTTTTTAAT
AGTTCTGCTACCCCCTCTTCTAATGGCTCCAAAAGCAGTTTTAATTTTAGCTTCAATAAT
AGCTCTACGCCGAGCTTCTCCTTTGTTAATGGCAGTGGCGTGAACAATCTGTACTCCTCG
TCAGCAAAATACAACTCCAACGATGAGGACCATCATTTGGATAGAGGCCCTGATATCAAA
CATAATCTAAAGTGCACATTGAAGGAACTCTACATGGGTAAGACTGCAAAGTTGGGTTTG
AATAGGACAAGGATTTGCAGTGTTTGTGATGGGCACGGTGGTCTAAAGAAATGCACTTGT
AAAACATGCAAAGGGCAAGGTATTCAAACCCAAACTAGGCGTATGGGACCTCTAGTACAA
AGTTGGTCTCAAACTTGTGCAGATTGCGGGGGTGCCGGGGTTTTTGTCAAAAATAAAGAT
ATTTGCCAACAGTGCCAAGGTCTTGGCTTCATTAAGGAGAGGAAGATTCTACAAGTCACC
GTTCAACCGGGATCGTGTCATAACCAACTTATAGTACTTACGGGCGAAGGTGACGAAGTT
ATTAGTACTAAGGGAGGCGGTCACGAAAAGGTAATACCTGGTGACGTCGTTATCACCATT
TTACGTTTAAAAGATCCGAATTTCCAGGTTATCAACTACTCCAATTTGATATGTAAGAAG
TGCAAAATCGACTTCATGACCAGTTTATGTGGAGGCGTAGTTTATATTGAAGGGCACCCT
AGCGGTAAGTTGATCAAACTTGATATTATACCTGGCGAGATACTGAAGCCTGGTTGTTTC
AAGACTGTTGAGGACATGGGGATGCCCAAGTTTATCAACGGTGTTCGGAGCGGTTTCGGT
CATCTATATGTCAAATTCGATGTGACGTATCCAGAGAGACTGGAACCTGAAAATGCTAAG
AAAATACAAAATATTCTGGCTAATGATAAATACATTAAAGCAGAACGTTCCACCATGGAA
ACCGCAGATTCAGACTGCTATTGCGATTTGGAGAAGTCATATGACAGTGTGGAAGAGCAT
GTGTTAAGTAGCTTTGAGGCCCCTAATTTAAACAATGAAGTTATTGAAGACGACGACCTT
GGTGATTTGATTAATGAAAGAGATTCTCGGAAAAGGAACAACCGTCGATTCGACGAAAGT
AATATTAATAATAATAATGAAACGAAACGAAATAAATATTCTTCACCGGTAAGCGGTTTT
TATGACCATGATATTAATGGATATTGA

Further information on APJ1 can be obtained from the URL address http://db.yeastgenome.org/cgi-bin/singlepageformat?sgdid=S000005021

It will be appreciated that, by “APJ1”, we include fragments or variants thereof having equivalent APJ1-like activity.

SIS1 is another S. cerevisiae helper protein of interest for the present invention. It is a type II HSP40 co-chaperone that interacts with the HSP70 protein Ssa1p; not functionally redundant with Ydj1p due to due to substrate specificity; shares similarity with bacterial DnaJ proteins. A published protein sequence for the protein Sis1p is as follows:

MVKETKLYDLLGVSPSANEQELKKGYRKAALKYHPDKPTGDTEKFKEISE
AFEILNDPQKREIYDQYGLEAARSGGPSFGPGGPGGAGGAGGFPGGAGGF
SGGHAFSNEDAFNIFSQFFGGSSPFGGADDSGFSFSSYPSGGGAGMGGMP
GGMGGMHGGMGGMPGGFRSASSSPTYPEEETVQVNLPVSLEDLFVGKKKS
FKIGRKGPHGASEKTQIDIQLKPGWKAGTKITYKNQGDYNPQTGRRKTLQ
FVIQEKSHPNFKRDGDDLIYTLPLSFKESLLGFSKTIQTIDGRTLPLSRV
QPVQPSQTSTYPGQGMPTPKNPSQRGNLIVKYKVDYPISLNDAQKRAI
DENF*

SIS1 is encoded by a non-essential gene comprising an ORF that is 1.059 kbp in size and is located on chromosome XIV. A published nucleotide coding sequence of SIS1 is as follows, although it will be appreciated that the sequence can be modified by degenerate substitutions to obtain alternative nucleotide sequences which encode an identical protein product:

ATGGTCAAGGAGACAAAACTTTATGATTTACTTGGAGTATCTCCAAGTGCTAATGAGCAA
GAACTGAAAAAGGGTTATAGAAAAGCAGCTCTAAAATATCATCCAGATAAGCCAACAGGT
GACACAGAAAAGTTTAAGGAGATATCAGAGGCCTTTGAAATTTTAAATGATCCTCAAAAA
AGGGAAATATATGATCAATACGGTCTCGAGGCTGCTAGATCTGGTGGTCCAAGCTTTGGT
CCTGGTGGTCCTGGCGGTGCTGGAGGTGCTGGAGGCTTCCCTGGCGGTGCGGGCGGATTC
TCCGGAGGACATGCGTTCAGTAATGAGGATGCTTTCAATATTTTTTCACAATTCTTTGGC
GGCAGTTCCCCATTCGGTGGTGCTGATGACAGTGGCTTCAGTTTCTCTAGTTATCCATCT
GGCGGCGGTGCTGGTATGGGAGGTATGCCTGGAGGAATGGGAGGAATGCATGGCGGCATG
GGAGGTATGCCTGGCGGCTTTAGATCAGCATCAAGCTCTCCCACGTATCCAGAGGAAGAA
ACAGTTCAAGTTAATTTACCAGTTAGTCTAGAAGATTTGTTTGTTGGTAAAAAGAAGTCA
TTTAAAATTGGAAGAAAGGGCCCACATGGGGCCTCTGAAAAGACACAAATTGACATTCAA
TTAAAACCGGGTTGGAAAGCTGGTACCAAAATAACATACAAGAACCAGGGTGATTACAAT
CCTCAAACGGGCCGTAGAAAGACTTTGCAGTTTGTCATCCAGGAAAAGAGCCATCCAAAC
TTTAAAAGAGACGGTGATGACCTAATTTACACTCTGCCACTATCTTTCAAGGAATCATTG
TTAGGTTTTTCAAAAACTATCCAAACAATTGATGGCAGAACCTTACCTTTGTCGAGAGTA
CAGCCTGTCCAACCCTCACAAACTTCTACTTATCCTGGTCAAGGTATGCCAACTCCAAAG
AACCCATCTCAGAGAGGTAATTTGATTGTAAAATATAAAGTGGACTATCCAATATCACTA
AACGACGCTCAAAAACGTGCTATAGATGAAAATTTTTAA

Further information on SIS1 can be obtained from the URL address http://db.yeastgenome.org/cgi-bin/singlepageformat?sgdid=S000004952

It will be appreciated that, by “SIS1”, we include fragments or variants thereof having equivalent SIS1-like activity.

DJP1 is another S. cerevisiae helper protein of interest for the present invention. It is also known as ICS1 and PAS22. It is a J-domain-containing protein, required for peroxisomal protein import and involved in peroxisome assembly, homologous to E. coli DnaJ and is located in the cytoplasm. A published protein sequence for the protein Djp1p is as follows:

MVVDTEYYDLLGVSTTASSIEIKKAYRKKSIQEHPDKNPNDPTATERFQAISEAYQVLGD
DDLRAKYDKYGRKEAIPQGGFEDAAEQFSVIFGGDAFASYIGELMLLKNLQKTEELNAED
EAEKEKENVETMEESPADGKTNGTTNAVDAALGNTNEKDDKNKARTTSGNLTVHDGNKKN
EQVGAEAKKKKTKLEQFEEEQEVEKQKRVDQLSKTLIERLSILTESVYDDACKDSFKKKF
EEEANLLKMESFGLDILHTIGDVYYEKAEIFLASQNLFGMGGIFHSMKAKGGVFMDTLRT
VSAAIDAQNTMKELEKMKEASTNNEPLFDKDGNEQIKPTTEELAQQEQLLMGKVLSAAWH
GSKYEITSTLRGVCKKVLEDDSVSKKTLIRRAEAMKLLGEVFKKTFRTKVEQEEAQIFEE
LVAEATKKKRHT*

DJP1 is encoded by a non-essential gene comprising an ORF that is 1.299 kbp in size and is located on chromosome IX. A published nucleotide coding sequence of DJP1 is as follows, although it will be appreciated that the sequence can be modified by degenerate substitutions to obtain alternative nucleotide sequences which encode an identical protein product:

ATGGTTGTTGATACTGAGTATTACGATTTGTTAGGTGTGTCTACCACTGCATCTTCCATT
GAAATAAAAAAGGCCTATAGAAAGAAATCTATTCAAGAGCATCCTGATAAGAATCCCAAT
GACCCCACGGCTACCGAAAGGTTTCAAGCAATATCCGAAGCTTATCAAGTTTTAGGTGAC
GATGATCTTCGCGCAAAGTATGATAAGTATGGAAGAAAAGAAGCTATTCCTCAGGGCGGC
TTTGAAGATGCAGCTGAACAGTTCTCTGTCATCTTTGGTGGAGATGCGTTTGCCTCATAT
ATTGGCGAACTGATGCTATTAAAGAACCTACAGAAAACTGAGGAGCTAAATGCTGAAGAC
GAAGCTGAAAAGGAGAAGGAGAATGTGGAAACAATGGAAGAATCACCTGCAGACGGTAAG
ACGAATGGCACCACTAACGCTGTTGATGCAGCATTGGGCAATACTAACGAAAAAGATGAC
AAAAATAAGGCGAGGACAACTTCTGGTAATTTAACTGTACACGATGGAAACAAGAAAAAT
GAGCAGGTAGGAGCAGAAGCTAAGAAGAAGAAGACAAAATTAGAGCAGTTTGAGGAAGAA
CAAGAGGTAGAAAAGCAAAAAAGAGTAGACCAATTAAGCAAAACATTGATTGAAAGATTA
TCGATATTAACAGAAAGTGTCTATGATGATGCATGTAAAGATTCCTTTAAAAAAAAGTTC
GAAGAGGAAGCCAATCTTTTAAAGATGGAATCATTTGGTCTGGACATATTACACACAATA
GGCGACGTTTACTACGAAAAAGCTGAAATTTTTCTTGCATCCCAGAACCTGTTCGGAATG
GGTGGTATATTTCATTCTATGAAGGCTAAAGGGGGAGTATTTATGGATACACTAAGAACT
GTTTCGGCAGCCATAGACGCTCAGAATACTATGAAGGAGCTTGAAAAAATGAAAGAAGCT
AGCACGAATAATGAGCCTTTGTTTGACAAAGACGGAAATGAGCAAATTAAGCCAACCACT
GAGGAACTGGCGCAGCAAGAGCAGCTATTGATGGGCAAAGTATTGTCGGCTGCTTGGCAT
GGTTCTAAATATGAAATAACATCCACTTTACGTGGCGTTTGTAAAAAAGTACTAGAAGAT
GACTCGGTAAGTAAGAAAACGCTTATCAGAAGAGCTGAAGCAATGAAACTATTGGGTGAA
GTCTTTAAGAAAACTTTCAGAACCAAAGTCGAACAAGAAGAGGCACAGATCTTTGAAGAA
CTTGTAGCAGAAGCTACAAAAAAGAAGAGACATACATGA

Further information on DJP1 can be obtained from the URL address http://db.yeastgenome.org/cgi-bin/singlepageformat?sgdid=S000001443

It will be appreciated that, by “DJP1”, we include fragments or variants thereof having equivalent DJP1-like activity.

ZUO1 is another S. cerevisiae helper protein of interest for the present invention. It is a cytosolic ribosome-associated chaperone that acts, together with Ssz1p and the Ssb proteins, as a chaperone for nascent polypeptide chains; contains a DnaJ domain and functions as a J-protein partner for Ssb1p and Ssb2p. A published protein sequence for the protein Zuo1p is as follows:

MFSLPTLTSDITVEVNSSATKTPFVRRPVEPVGKFFLQHAQRTLRNHTWSEFERIEAEKN
VKTVDESNVDPDELLFDTELADEDLLTHDARDWKTADLYAAMGLSKLRFRATESQIIKAH
RKQVVKYHPDKQSAAGGSLDQDGFFKIIQKAFETLTDSNKRAQYDSCDFVADVPPPKKGT
DYDFYEAWGPVFEAEARFSKKTPIPSLGNKDSSKKEVEQFYAFWHRFDSWRTFEFLDEDV
PDDSSNRDHKRYIERKNKAARDKKKTADNARLVKLVERAVSEDPRIKMFKEEEKKEKERR
KWEREAGARAEAEAKAKAEAEAKAKAESEAKANASAKADKKKAKEAAKAAKKKNKRAIRN
SAKEADYFGDADKATTIDEQVGLIVDSLNDEELVSTADKIKANAAGAKEVLKESAKTIVD
SGKLPSSLLSYFV*

ZUO1 is encoded by a non-essential gene comprising an ORF that is 1.302 kbp in size and is located on chromosome VII. A published nucleotide coding sequence of ZUO1 is as follows, although it will be appreciated that the sequence can be modified by degenerate substitutions to obtain alternative nucleotide sequences which encode an identical protein product:

ATGTTTTCTTTACCTACCCTAACCTCAGACATCACTGTTGAAGTCAACAGTTCCGCTACC
AAAACCCCATTCGTCCGTCGTCCGGTCGAACCGGTTGGTAAGTTCTTTTTGCAACATGCT
CAAAGAACTTTGAGAAACCACACCTGGTCTGAATTTGAAAGAATTGAAGCTGAAAAGAAC
GTCAAAACCGTTGATGAATCCAATGTCGACCCAGATGAGTTGTTATTCGACACTGAATTG
GCCGATGAAGATTTACTGACTCATGATGCTAGAGACTGGAAAACTGCCGATTTGTATGCT
GCTATGGGTTTGTCTAAGTTGCGTTTCAGAGCTACTGAAAGTCAAATCATCAAGGCTCAC
AGAAAACAAGTTGTCAAGTACCATCCAGACAAGCAATCTGCTGCTGGTGGTAGTTTGGAC
CAAGATGGCTTTTTCAAGATTATTCAAAAGGCCTTTGAAACTTTGACTGATTCCAACAAG
AGAGCTCAGTACGACTCATGTGATTTTGTTGCCGATGTTCCTCCTCCAAAGAAGGGTACC
GATTATGACTTTTATGAAGCTTGGGGCCCCGTTTTCGAAGCTGAAGCTCGTTTTTCTAAG
AAGACTCCTATTCCTTCTCTAGGTAACAAAGATTCTTCCAAGAAGGAAGTTGAACAATTC
TATGCTTTCTGGCACAGATTTGACTCCTGGAGAACCTTTGAGTTCTTGGACGAAGATGTC
CCAGATGACTCTTCTAACAGAGACCACAAGCGTTACATTGAAAGAAAGAACAAGGCCGCA
AGAGACAAGAAGAAGACTGCTGATAACGCTAGATTGGTCAAACTTGTTGAAAGAGCTGTC
AGTGAAGATCCCCGTATCAAAATGTTCAAAGAAGAAGAGAAGAAGGAAAAGGAAAGAAGA
AAATGGGAAAGAGAAGCCGGTGCCAGAGCTGAAGCTGAAGCTAAGGCCAAGGCCGAAGCT
GAAGCGAAGGCTAAAGCTGAATCTGAAGCCAAGGCTAACGCCTCCGCAAAAGCTGACAAA
AAGAAGGCTAAGGAAGCTGCTAAGGCCGCCAAGAAAAAGAACAAGAGAGCCATCCGTAAC
TCTGCTAAGGAAGCTGACTACTTTGGTGATGCTGACAAGGCCACCACGATTGACGAACAA
GTTGGTTTGATCGTTGACAGTTTGAATGACGAAGAGTTAGTGTCCACCGCCGATAAGATC
AAGGCCAATGCTGCTGGTGCCAAGGAAGTTTTGAAGGAATCTGCAAAGACTATTGTCGAT
TCTGGCAAACTACCATCCAGCTTGTTGTCCTACTTCGTGTGA

Further information on ZUO1 can be obtained from the URL address http://db.yeastgenome.org/cgi-bin/singlepageformat?sgdid=S000003517

It will be appreciated that, by “ZUO1”, we include fragments or variants thereof having equivalent ZUO1-like activity.

SWA2 is another S. cerevisiae helper protein of interest for the present invention. It is also known as AUX1 and BUD24. It is an auxilin-like protein involved in vesicular transport; clathrin-binding protein required for uncoating of clathrin-coated vesicles. A published protein sequence for the protein Swa2p is as follows:

MSDPFAHLLTSLKNKDSASASKETTPQSSNSPSITGSAVADVARTDKSPNDSLHSISAPP
LIPSPKVDFSAPPLVPTNSTTKSNTANNTPPSALANTDDDFNQLFGMGTVTTTDTIQKPD
EDYYGSKEDHLYNGDDALVDEVKDMEIARLMSLGLSIEEATEFYENDVTYERYLEILKSK
QKERNDLAIRKKESGIKMEKSGLSNIVGTDSNNLFSMATDFFNKGKKLVDQWTSFPPEAN
DRLNNYSKTHDKVEDYDLPQVNDSPNRILFEDNEVVENLPPADNPDQDLLTDFETKIDIT
KRTAPDVSHSSSPTSGILIEENSRRNEPLIEDSLLDFSEGNLTNSKSNEDSTLFNENSNT
DSTIPISDIELSGYNEFKAKGTSLFKNGDYINSLQEYEKSLNTLPLNHPLRIIALSNIIA
SQLKIGEYSKSIENSSMALELFPSSKAKWKNKISNSDPERSFNDIWPKIMIRRAESFEHL
ESFKKALETYQELIKKNFFDDKIMQGKRRCQDFINPPPVKKSMPVKKKTTTTSPATKKQN
LTASSSNSPISVDSTSEIKKRELENAKLALYDKVFEKISSWKDGKDDDIRHLLANLSSLL
TWCNWKDVSMQDLVMPKRVKITYMKAVAKTHPDKIPESLSLENKMIAENIFSTLSIAWDK
FKLQNDIN*

SWA2 is encoded by a non-essential gene comprising an ORF that is 2.007 kbp in size and is located on chromosome IV. A published nucleotide coding sequence of SWA2 is as follows, although it will be appreciated that the sequence can be modified by degenerate substitutions to obtain alternative nucleotide sequences which encode an identical protein product:

ATGTCAGATCCATTTGCACATTTACTGACTTCTTTGAAGAATAAGGACTCTGCATCTGCA
TCCAAGGAAACAACTCCTCAGAGCAGCAATTCGCCTTCCATTACTGGTTCCGCTGTTGCA
GATGTTGCAAGGACGGATAAAAGCCCCAATGATAGTCTGCATTCAATTTCAGCTCCTCCG
CTGATACCGTCACCGAAGGTAGATTTTTCTGCACCTCCTTTGGTCCCAACTAATAGCACC
ACTAAATCTAATACTGCCAACAACACACCTCCCTCGGCTCTTGCCAATACCGATGACGAC
TTCAATCAACTATTTGGTATGGGCACAGTAACTACAACGGATACGATCCAAAAACCGGAT
GAGGATTACTATGGAAGCAAGGAAGACCACCTTTACAATGGTGATGACGCCTTAGTTGAT
GAAGTTAAGGATATGGAAATAGCAAGATTGATGTCTCTAGGTTTATCAATTGAAGAAGCC
ACTGAGTTTTACGAAAATGACGTAACTTATGAAAGATATTTGGAGATTTTAAAGTCAAAG
CAAAAGGAGCGCAACGATCTAGCTATAAGAAAGAAAGAAAGTGGTATAAAAATGGAAAAG
TCAGGATTATCCAACATTGTTGGTACAGATAGCAATAATTTATTCAGCATGGCCACTGAT
TTTTTCAATAAGGGTAAGAAACTGGTAGACCAATGGACCTCCTTCCCACCTGAGGCAAAT
GATAGACTGAATAATTACTCAAAAACTCATGATAAGGTTGAGGATTATGATTTGCCTCAA
GTAAACGACTCACCCAATAGAATTTTGTTTGAAGATAATGAAGTCGTAGAGAACTTACCA
CCTGCCGATAATCCGGATCAAGATCTTTTAACTGATTTCGAAACAAAGATTGATATAACA
AAGAGGACAGCGCCTGATGTCTCCCACTCCTCCTCACCGACTTCTGGTATACTAATTGAA
GAAAATTCGCGAAGAAATGAGCCCCTGATAGAGGATAGTCTTCTCGACTTTTCAGAAGGA
AATCTCACCAATAGTAAAAGCAATGAAGATAGCACCCTCTTCAATGAAAACAGCAACACT
GACTCTACAATACCCATCTCAGATATTGAATTATCGGGGTATAACGAATTTAAGGCGAAA
GGTACTAGTTTGTTCAAGAACGGGGATTATATTAACTCATTACAAGAATATGAAAAGTCT
TTAAATACATTGCCTTTAAATCATCCATTGAGGATCATTGCATTATCAAACATTATTGCC
TCGCAACTGAAAATCGGTGAGTACTCTAAGTCCATAGAAAACTCCAGCATGGCTTTGGAA
TTATTCCCATCAAGCAAAGCTAAGTGGAAGAATAAAATCTCAAATAGTGACCCTGAAAGA
TCATTTAACGACATCTGGCCAAAGATTATGATTAGGCGTGCTGAGTCTTTTGAACATTTA
GAAAGTTTCAAAAAAGCACTAGAAACATACCAAGAGCTGATTAAGAAGAATTTTTTTGAT
GATAAAATCATGCAGGGAAAAAGAAGATGCCAAGACTTTATTAATCCTCCCCCTGTTAAA
AAATCCATGCCCGTTAAGAAGAAGACAACGACAACCTCGCCTGCAACAAAAAAACAGAAC
TTAACCGCTTCTTCTTCAAATTCTCCAATTTCTGTTGATAGCACTTCAGAAATAAAAAAA
CGGGAGCTAGAAAACGCTAAACTGGCGCTATATGATAAAGTATTTGAGAAAATTAGCTCC
TGGAAGGATGGCAAAGACGATGACATTCGTCATCTGTTAGCAAATTTATCCAGCTTACTA
ACATGGTGCAATTGGAAGGATGTCTCTATGCAAGATTTGGTTATGCCTAAGAGGGTCAAA
ATTACATACATGAAAGCTGTAGCCAAGACACATCCTGATAAGATACCAGAGTCCTTGTCC
CTGGAAAATAAGATGATTGCAGAGAATATTTTCAGTACTTTAAGTATTGCTTGGGATAAG
TTCAAACTGCAGAATGACATTAACTGA

Further information on SWA2 can be obtained from the URL address http://db.yeastgenome.org/cgi-bin/singlepageformat?sgdid=S000002728

It will be appreciated that, by “SWA2”, we include fragments or variants thereof having equivalent SWA2-like activity.

JJJ1 is another S. cerevisiae helper protein of interest for the present invention. It contains a 70 amino acid J-domain, may function as a co-chaperone to recruit Hsp70-like activity to specific sites; mutation of it causes defects in fluid-phase endocytosis. A published protein sequence for the protein Jjj1p is as follows:

MKTCYYELLGVETHASDLELKKAYRKKALQYHPDKNPDNVEEATQKFAVIRAAYEVLSDP
QERAWYDSHKEQILNDTPPSTDDYYDYEVDATVTGVTTDELLLFFNSALYTKIDNSAAGI
YQIAGKIFAKLAKDEILSGKRLGKFSEYQDDVFEQDINSIGYLKACDNFINKTDKLLYPL
FGYSPTDYEYLKHFYKTWSAFNTLKSFSWKDEYMYSKNYDRRTKREVNRRNEKARQQARN
EYNKTVKRFVVFIKKLDKRMKEGAKIAEEQRKLKEQQRKNELNNRRKFGNDNNDEEKFHL
QSWQTVKEENWDELEKVYDNFGEFENSKNDKEGEVLIYECFICNKTFKSEKQLKNHINTK
LHKKNMEEIRKEMEEENITLGLDNLSDLEKFDSADESVKEKEDIDLQALQAELAEIERKL
AESSSEDESEDDNLNIEMDIEVEDVSSDENVHVNTKNKKKRKKKKKAKVDTETEESESFD
DTKDKRSNELDDLLASLGDKGLQTDDDEDWSTKAKKKKGKQPKKNSKSTKSTPSLSTLPS
SMSPTSAIEVCTTCGESFDSRNKLFNHVKIAGHAAVKNVVKRKKVKTKRI*

JJJ1 is encoded by a non-essential gene comprising an ORF that is 1.773 kbp in size and is located on chromosome XIV. A published nucleotide coding sequence of JJJ1 is as follows, although it will be appreciated that the sequence can be modified by degenerate substitutions to obtain alternative nucleotide sequences which encode an identical protein product:

ATGAAGACCTGCTACTATGAGCTTTTAGGGGTCGAAACGCATGCTTCTGATCTTGAGTTA
AAAAAAGCTTACCGTAAAAAGGCCCTACAATATCACCCAGATAAAAACCCAGATAATGTT
GAAGAAGCCACACAAAAATTTGCTGTGATTCGAGCCGCTTATGAAGTACTGTCTGACCCC
CAGGAAAGAGCATGGTATGACTCACATAAGGAACAAATTTTAAATGATACTCCACCAAGC
ACTGATGATTACTATGATTATGAGGTAGACGCTACAGTCACAGGTGTCACAACTGATGAA
TTACTCTTATTTTTTAACTCTGCTCTTTATACTAAAATAGACAACTCAGCTGCTGGGATA
TATCAAATTGCAGGAAAAATATTTGCCAAGTTAGCTAAAGATGAGATTTTAAGTGGTAAG
CGACTGGGGAAATTTTCCGAGTATCAAGATGATGTATTCGAACAGGATATTAATAGTATT
GGCTATTTGAAAGCCTGCGATAACTTTATTAACAAGACGGATAAACTTTTATATCCTTTA
TTTGGATATTCGCCAACGGATTATGAATATTTGAAACATTTCTATAAGACTTGGTCAGCG
TTCAATACCTTGAAAAGTTTTAGCTGGAAAGACGAGTACATGTACTCTAAAAACTATGAC
AGAAGAACCAAGAGGGAAGTTAATAGAAGAAATGAGAAGGCTAGGCAACAAGCTCGAAAT
GAATACAACAAAACCGTGAAAAGGTTTGTAGTTTTCATAAAAAAGCTCGATAAAAGAATG
AAAGAAGGTGCAAAAATTGCAGAAGAACAGCGTAAACTAAAAGAACAACAGAGGAAAAAT
GAGTTAAATAACAGAAGAAAGTTTGGGAACGACAACAATGACGAAGAAAAATTTCATTTA
CAAAGCTGGCAAACGGTAAAAGAAGAAAACTGGGATGAACTGGAAAAGGTATATGATAAT
TTTGGAGAATTCGAAAATTCTAAGAATGATAAGGAAGGTGAAGTATTGATTTACGAGTGT
TTTATCTGCAACAAGACATTTAAGTCGGAAAAGCAATTGAAAAACCACATAAACACTAAA
CTGCATAAGAAAAATATGGAAGAGATACGGAAAGAAATGGAAGAGGAAAACATAACGCTT
GGGTTGGATAATCTCTCCGATCTCGAGAAATTTGATTCAGCAGATGAAAGTGTTAAAGAA
AAAGAAGATATTGATCTGCAAGCATTGCAAGCTGAACTCGCTGAAATTGAAAGAAAACTG
GCAGAATCGTCTTCTGAAGACGAAAGTGAAGATGACAATCTCAACATAGAAATGGATATA
GAGGTAGAAGACGTCAGTTCGGATGAAAATGTACATGTGAATACGAAGAATAAAAAGAAA
AGAAAAAAGAAAAAAAAAGCAAAGGTTGACACAGAAACAGAGGAATCTGAATCGTTCGAT
GATACTAAAGACAAACGGAGTAATGAGTTGGATGATCTTTTGGCATCACTAGGAGACAAG
GGCTTACAAACGGATGACGATGAAGATTGGTCTACTAAAGCGAAAAAGAAAAAGGGCAAA
CAACCTAAAAAGAATTCTAAATCCACAAAAAGCACTCCGTCCTTGTCGACTCTACCGTCC
TCTATGTCTCCAACCTCCGCGATCGAGGTGTGCACTACATGCGGAGAATCATTTGATAGT
CGAAATAAGCTATTCAACCACGTGAAGATAGCAGGGCATGCGGCAGTGAAAAACGTAGTG
AAAAGAAAGAAAGTCAAGACCAAAAGAATATAG

Further information on JJJ1 can be obtained from the URL address http://db.yeastgenome.org/cgi-bin/singlepageformat?sgdid=S000005171

It will be appreciated that, by “JJJ1”, we include fragments or variants thereof having equivalent JJJ1-like activity.

JJJ2 is another S. cerevisiae helper protein of interest for the present invention. It is one of several homologs of the bacterial chaperone DnaJ, and is located in the cytoplasm. A published protein sequence for the protein Jjj2p is as follows:

MSQVIEPQLDRTTYYSILGLTSNATSSEVHKSYLKLARLLHPDKTKSDKSEELFKAVVHA
HSILTDEDQKLRYDRDLKIKGLHTYQPKKNCHIFKTKAKESQGASPTLGQSEAYHRQNKP
YEQQPYGFGVGKKMTSSSKSKVPIFKSFNLKSYQRNHYYSSKKERKHGSPDIDSLFHETN
GASKVRMTDAGKMDTNSQFQEIWEILGKNAYTHKSYSEDPNSCLGSALSDHEEEEEAGKQ
QQQQQQQQQQQQHYGMTSKSSSPDEEKKNNKEPKRESRVSPEENGEEETGHKQFKLPKTS
TFSSGSHDSNLQSPFYNHEYRHYARSKFECKNQFRKSVSPIKEIPATTSANEGWNILRDI
IEKLNISNVDDRNKDLLFRRDEIGDKNHSDSIDIENLSIKEPKGMKRRKKDDISLEELFQ
SLPREKDYFMMDAINDSLESINLFKKPKTTQSHEQGGTFAQAESNRAKFKPLLEQCGITP
EILDLEIPEIPEFDAVADLETLKLNVQLFNNQCNKLKETIHQVSLQRLRADTQFSDMLTQ
KQSIMVWKTYLEFDKSLMDKLNILQERQMQVIKIFSERCDGKV*

JJJ2 is encoded by a non-essential gene comprising an ORF that is 1.752 kbp in size and is located on chromosome 10. A published nucleotide coding sequence of JJJ2 is as follows, although it will be appreciated that the sequence can be modified by degenerate substitutions to obtain alternative nucleotide sequences which encode an identical protein product:

ATGTCACAGGTAATAGAACCACAATTAGATAGAACAACCTATTATTCCATATTAGGCTTG
ACATCAAATGCGACTTCCTCCGAAGTACATAAATCATATCTAAAACTGGCCAGATTACTT
CACCCAGATAAAACAAAATCTGATAAGTCTGAGGAATTATTCAAAGCTGTGGTGCATGCA
CATTCAATTTTAACTGATGAAGATCAAAAACTTCGATATGATCGAGATTTGAAAATCAAA
GGTTTACACACTTACCAGCCGAAGAAAAACTGTCATATTTTCAAGACCAAGGCAAAGGAA
TCACAAGGGGCTAGTCCCACACTTGGTCAATCAGAAGCTTATCATAGGCAAAATAAACCT
TATGAGCAACAGCCCTACGGTTTCGGTGTAGGCAAAAAAATGACCTCAAGCTCTAAGAGT
AAGGTTCCGATATTCAAGTCCTTCAATTTAAAAAGCTACCAACGAAACCACTATTATTCA
TCCAAAAAGGAAAGGAAACATGGAAGTCCTGATATTGATTCTTTGTTCCATGAAACCAAT
GGAGCCTCAAAAGTAAGAATGACTGATGCCGGTAAAATGGATACGAACTCTCAGTTCCAA
GAAATATGGGAAATATTGGGTAAAAATGCGTACACACATAAATCTTACTCTGAAGATCCA
AATTCATGTTTGGGATCAGCACTAAGCGATCATGAAGAAGAAGAAGAAGCAGGAAAACAA
CAACAGCAACAGCAGCAACAACAGCAACAGCAGCAACATTATGGAATGACGTCGAAGTCT
AGCAGTCCTGATGAAGAAAAAAAAAATAATAAAGAACCGAAAAGGGAAAGCAGAGTCTCT
CCAGAGGAAAATGGCGAAGAAGAAACGGGACACAAACAATTTAAATTGCCCAAGACCAGT
ACTTTTTCTAGTGGATCCCATGATTCAAATTTGCAATCTCCTTTTTACAATCATGAGTAT
CGACATTACGCAAGAAGTAAATTCGAATGCAAGAATCAGTTTAGAAAGTCAGTTTCTCCC
ATTAAAGAGATACCTGCAACAACTAGTGCCAATGAAGGATGGAACATTTTGAGAGACATT
ATTGAAAAACTCAATATAAGCAATGTAGACGATCGAAATAAAGACTTGCTGTTTCGTCGG
GATGAAATAGGTGATAAAAATCACAGCGACTCAATCGACATAGAAAATTTATCTATCAAA
GAACCTAAAGGGATGAAAAGGAGAAAGAAAGATGATATATCTTTAGAAGAATTGTTCCAA
TCTTTACCAAGAGAAAAAGATTATTTTATGATGGATGCAATTAATGACTCGTTAGAATCA
ATCAATCTTTTTAAAAAGCCGAAGACCACTCAGAGTCACGAACAAGGTGGAACTTTTGCC
CAAGCAGAAAGTAATCGTGCAAAATTCAAACCGTTACTAGAACAGTGTGGAATTACACCC
GAGATCTTAGATTTGGAAATACCAGAGATTCCGGAATTTGATGCAGTGGCTGACCTTGAA
ACATTGAAGCTTAACGTGCAGCTGTTTAATAACCAATGTAACAAACTTAAAGAAACAATA
CATCAAGTATCATTACAGCGCCTGAGAGCAGATACGCAGTTCAGTGATATGTTAACCCAA
AAGCAAAGTATTATGGTTTGGAAAACATACCTAGAATTTGATAAAAGTTTAATGGACAAA
TTGAACATCTTACAAGAAAGACAGATGCAGGTCATTAAAATTTTTTCCGAAAGATGTGAC
GGTAAAGTATAA

Further information on JJJ2 can be obtained from the URL address http://db.yeastgenome.org/cgi-bin/singlepageformat?sgdid=S000003698

It will be appreciated that, by “JJJ2”, we include fragments or variants thereof having equivalent JJJ2-like activity.

JJJ3 is another S. cerevisiae helper protein of interest for the present invention and is also known as DPH4. It is one of several homologs of the bacterial chaperone DnaJ, and is located in the cytoplasm. A published protein sequence for the protein Jjj3p is as follows:

MSLVNSLTHYEILRIPSDATQDEIKKAYRNRLLNTHPDKLSKSIHDTVSN
VTINKIQDAYKILSNIKTRREYDRLILENYKRQGFHNCGDGLDEFSLDDF
SFDEDKLEFMMNCPRCQFVGGFHFSESLLDECIDNVDAMERSHSGYQ
LLTQCSACSLWLKVNFDIEEEQEGQ

JJJ3 is encoded by a non-essential gene comprising an ORF that is 0.519 kbp in size and is located on chromosome X. A published nucleotide coding sequence of JJJ3 is as follows, although it will be appreciated that the sequence can be modified by degenerate substitutions to obtain alternative nucleotide sequences which encode an identical protein product:

ATGTCATTGGTGAATTCGTTAACACACTACGAAATTTTAAGAATTCCATCGGATGCAACA
CAAGATGAAATCAAAAAGGCATATAGGAATCGGTTACTAAATACGCACCCCGATAAACTT
TCTAAAAGCATACATGATACGGTTAGCAACGTCACAATCAATAAGATTCAAGATGCTTAT
AAAATACTATCGAATATAAAAACTCGTCGCGAATATGATAGGTTGATCCTTGAAAACTAT
AAACGCCAAGGATTTCATAATTGTGGTGATGGGCTGGATGAATTTTCCTTAGACGATTTC
TCATTTGATGAAGATAAGCTGGAGTTTATGATGAATTGTCCTCGCTGTCAATTTGTTGGT
GGTTTTCATTTTAGTGAGAGTTTGTTAGATGAATGCATTGATAATGTAGACGCTATGGAA
CGGAGTCATTCTGGTTATCAATTATTAACCCAATGTAGCGCATGCAGCTTATGGCTGAAG
GTTAATTTTGACATCGAGGAAGAGCAAGAAGGACAATAA

Further information on JJJ3 can be obtained from the URL address http://db.yeastgenome.org/cgi-bin/singlepageformat?sgdid=S000003858

It will be appreciated that, by “JJJ3”, we include fragments or variants thereof having equivalent JJJ3-like activity.

CAJ1 is another S. cerevisiae helper protein of interest for the present invention. It is one of several homologs of the bacterial chaperone DnaJ, and is located in the nucleus. A published protein sequence for the protein Caj1p is as follows:

MVKETEYYDILGIKPEATPTEIKKAYRRKAMETHPDKHPDDPDAQAKFQA
VGEAYQVLSDPGLRSKYDQFGKEDAVPQQGFEDASEYFTAIFGGDGFKDW
IGEFSLFKELNEATEMFGKEDEEGTAATETEKADESTDGGMVKHDTNKAE
SLKKDKLSKEQREKLMEMEKKRREDMMKQVDELAEKLNEKISRYLIAVK
SNNLEEFTRKLDQEIEDLKLESFGLELLYLLARVYKTKANNFIMSKKTY
GISKIFTGTRDNARSVKSAYNLLSTGLEAQKAMEKMSEVNTDELDQYERA
KFESTMAGKALGVMWAMSKFELERKLKDVCNKILNDKKVPSKERIAKAK
AMLFIAHKFASARRSPEEAEEARVFEELILGEQEKEHKKHTVAR

CAJ1 is encoded by a non-essential gene comprising an ORF that is 1.176 kbp in size and is located on chromosome V. A published nucleotide coding sequence of CAJ1 is as follows, although it will be appreciated that the sequence can be modified by degenerate substitutions to obtain alternative nucleotide sequences which encode an identical protein product:

ATGGTAAAGGAGACGGAGTATTATGATATTTTGGGCATCAAGCCTGAGGCCACGCCCACT
GAAATCAAAAAGGCCTATCGTAGAAAGGCTATGGAAACACATCCGGACAAGCATCCTGAT
GACCCAGATGCTCAAGCAAAGTTTCAAGCCGTAGGCGAGGCCTACCAAGTCTTAAGTGAT
CCAGGGCTTCGTTCCAAGTATGACCAGTTTGGTAAGGAGGATGCTGTTCCTCAGCAAGGA
TTTGAAGATGCTTCTGAATACTTTACAGCAATATTCGGTGGTGATGGCTTCAAAGATTGG
ATTGGAGAATTTTCTTTGTTCAAAGAGCTAAACGAGGCAACAGAAATGTTTGGAAAGGAA
GATGAGGAGGGTACAGCAGCCACTGAAACCGAAAAAGCAGATGAGAGCACTGATGGTGGA
ATGGTTAAGCATGACACTAATAAAGCTGAATCTTTGAAAAAAGATAAATTATCGAAGGAG
CAAAGAGAGAAGCTAATGGAAATGGAGAAAAAAAGACGGGAAGATATGATGAAACAAGTC
GACGAGTTGGCAGAAAAACTGAACGAAAAAATCTCTAGGTACTTAATTGCTGTGAAGTCC
AATAACTTGGAGGAATTTACGCGAAAACTAGATCAAGAAATCGAGGATTTGAAATTAGAA
AGTTTTGGTCTAGAGTTATTGTATTTATTGGCCAGGGTTTACAAGACAAAAGCGAATAAT
TTTATCATGTCCAAGAAGACTTACGGAATTTCTAAAATATTCACTGGTACACGCGACAAT
GCTAGATCTGTTAAATCAGCATACAATTTATTGTCTACAGGCTTAGAAGCTCAAAAAGCC
ATGGAAAAAATGAGTGAAGTCAATACTGACGAACTAGACCAATATGAACGTGCCAAATTT
GAGTCCACAATGGCTGGTAAGGCACTTGGTGTCATGTGGGCTATGTCGAAATTTGAACTG
GAAAGAAAACTAAAAGACGTTTGCAATAAGATTCTAAACGATAAAAAGGTCCCTTCCAAG
GAACGTATTGCAAAGGCAAAAGCAATGCTGTTTATTGCCCACAAGTTTGCCAGTGCTAGA
AGGTCACCAGAAGAAGCTGAAGAAGCTAGAGTTTTTGAAGAGCTAATCCTAGGTGAGCAG
GAGAAGGAACACAAAAAACATACTGTGGCCAGATAA

Further information on CAJ1 can be obtained from the URL address http://db.yeastgenome.org/cgi-bin/singlepageformat?sgdid=S000000850

It will be appreciated that, by “CAJ1”, we include fragments or variants thereof having equivalent CAJ1-like activity.

CWC23 is another S. cerevisiae helper protein of interest for the present invention. It is one of several homologs of the bacterial chaperone DnaJ, and is located in the nucleus. A published protein sequence for the protein Cwc23p is as follows:

MPGHELEDVINQRLNLYDVLELPTPLDVHTIYDDLPQIKRKYRTLALKYH
PDKHPDNPSIIHKFHLLSTATNILTNADVRPHYDRWLIEFLRKTNDIERN
KLIQKLEESESSTIPTTTPHPDLLQIQRHGELLRKLKHFNLPYGDWKHLN
TQDQENASQHPYYDCSTLRIVLDNFLQSNNKSNCLSHLRNQVFITLSANE
IYDIYFSERNNYSKDDSIIIYTVFDTPITAQHVFRNWSSGNLIPTVKDIS
PLIPLHYYSDFNLETELNDDIARLVSNEPILLD

CWC23 is encoded by an essential gene comprising an ORF that is 0.852 kbp in size and is located on chromosome VII. A published nucleotide coding sequence of CWC23 is as follows, although it will be appreciated that the sequence can be modified by degenerate substitutions to obtain alternative nucleotide sequences which encode an identical protein product:

ATGCCAGGACACGAATTGGAAGACGTAATAAATCAACGTTTGAACCTATATGATGTATTA
GAATTACCGACCCCCCTGGACGTCCATACCATCTACGATGATTTGCCCCAAATTAAACGC
AAATACAGGACCCTTGCCCTGAAGTATCATCCTGACAAACACCCGGACAATCCATCAATT
ATACACAAATTCCACTTATTATCGACCGCAACTAATATCCTCACCAATGCAGACGTGAGA
CCCCATTACGACCGCTGGTTAATTGAGTTCCTACGGAAAACAAACGACATTGAAAGAAAT
AAACTTATACAAAAGCTGGAAGAATCTGAATCGAGTACGATACCCACCACCACACCACAT
CCTGATTTATTGCAAATCCAACGCCACGGCGAGCTACTCAGGAAACTAAAACATTTCAAC
TTGCCCTATGGTGACTGGAAACATCTCAACACACAAGACCAAGAAAATGCTTCGCAACAT
CCGTATTACGATTGCTCTACTTTGAGAATTGTCCTTGACAACTTCCTGCAATCAAATAAT
AAATCAAACTGCTTATCTCATTTGCGCAATCAAGTATTCATCACGCTAAGTGCTAATGAA
ATCTACGACATCTACTTCTCTGAAAGAAACAACTACTCGAAGGATGATTCAATCATCATA
TATACTGTATTCGATACTCCCATCACAGCGCAGCACGTATTCCGAAACTGGTCAAGTGGG
AACCTCATACCCACGGTCAAGGATATTTCGCCCTTGATCCCGCTACATTACTACTCTGAT
TTTAATTTGGAGACGGAACTGAATGACGATATTGCAAGACTGGTCTCTAATGAACCTATC
CTACTCGACTAG

Further information on CWC23 can be obtained from the URL address http://db.yeastgenome.org/cgi-bin/singlepageformat?sgdid=S000003096

It will be appreciated that, by “CWC23”, we include fragments or variants thereof having equivalent CWC23-like activity.

PAM18 is another S. cerevisiae helper protein of interest for the present invention and is also known as TIM14. It is one of several homologs of the bacterial chaperone DnaJ, and is located in the mitochondria. A published protein sequence for the protein Pam18p is as follows:

MSSQSNTGNSIEAPQLPIPGQTNGSANVTVDGAGVNVGIQNGSQGQKTGM
DLYFDQALNYMGEHPVITGFGAFLTLYFTAGAYKSISKGLNGGKSTTAFL
KGGFDPKMNSKEALQILNLTENTLTKKKLKEVHRKIMLANHPDKGGSPFL
ATKINEAKDFLEKRGISK

PAM18 is encoded by an essential gene comprising an ORF that is 0.507 kbp in size and is located on chromosome XII. A published nucleotide coding sequence of PAM18 is as follows, although it will be appreciated that the sequence can be modified by degenerate substitutions to obtain alternative nucleotide sequences which encode an identical protein product:

ATGAGTTCTCAAAGTAATACTGGTAATTCTATTGAGGCACCACAACTACCCATTCCTGGT
CAAACTAATGGCTCTGCGAACGTTACTGTTGATGGAGCTGGTGTTAATGTCGGTATCCAG
AATGGTTCGCAGGGTCAAAAGACCGGAATGGACCTTTATTTTGATCAAGCTTTGAACTAC
ATGGGAGAACATCCTGTGATAACAGGTTTTGGGGCCTTTTTAACTTTATATTTTACAGCC
GGTGCATATAAATCAATATCGAAGGGACTTAACGGTGGAAAATCCACTACTGCCTTCTTG
AAAGGCGGATTTGACCCGAAAATGAATTCTAAAGAGGCTCTACAGATTTTGAATTTGACA
GAAAATACATTGACTAAAAAAAAGTTGAAAGAGGTTCATAGGAAAATTATGTTAGCTAAT
CATCCTGACAAAGGTGGTTCTCCATTTTTGGCCACTAAGATAAACGAAGCTAAGGACTTT
TTGGAAAAAAGGGGTATTAGCAAATAA

Further information on PAM18 can be obtained from the URL address http://db.yeastgenome.org/cgi-bin/singlepageformat?sgdid=S000003998

It will be appreciated that, by “PAM18”, we include fragments or variants thereof having equivalent PAM18-like activity.

JAC1 is another S. cerevisiae helper protein of interest for the present invention. It is one of several homologs of the bacterial chaperone DnaJ, and is located in the mitochondria. A published protein sequence for the protein Jac1p is as follows:

MLKYLVQRRFTSTFYELFPKTFPKKLPIWTIDQSRLRKEYRQLQAQHHPD
MAQQGSEQSSTLNQAYHTLKDPLRRSQYMLKLLRNIDLTQEQTSNEVTTS
DPQLLLKVLDIHDELSQMDDEAGVKLLEKQNKERIQDIEAQLGQCYNDKD
YAAAVKLTVELKYWYNLAKAFKDWAPGKQLEMNH

JAC1 is encoded by an essential gene comprising an ORF that is 0.555 kbp in size and is located on chromosome VII. A published nucleotide coding sequence of JAC1 is as follows, although it will be appreciated that the sequence can be modified by degenerate substitutions to obtain alternative nucleotide sequences which encode an identical protein product:

ATGTTGAAATACTTGGTTCAACGAAGATTCACTTCTACATTTTACGAGCTGTTCCCAAAG
ACCTTCCCCAAAAAGCTACCCATTTGGACTATCGATCAATCCAGATTAAGGAAGGAGTAT
AGGCAATTACAAGCACAGCACCATCCAGACATGGCCCAACAAGGTAGTGAACAGTCATCA
ACTCTTAATCAAGCTTACCATACTCTCAAAGATCCCCTTAGAAGGTCACAATATATGCTA
AAACTCTTGCGCAATATCGATTTGACGCAAGAACAGACCTCAAATGAAGTAACTACCAGT
GATCCACAGTTACTATTGAAAGTTCTAGACATCCATGATGAATTATCCCAGATGGACGAC
GAAGCTGGTGTGAAGCTGCTTGAAAAGCAAAACAAGGAAAGAATTCAAGATATTGAAGCC
CAGTTGGGACAATGCTACAATGACAAGGATTACGCCGCCGCAGTGAAGTTGACCGTGGAG
CTAAAGTACTGGTACAACTTGGCCAAGGCATTCAAAGACTGGGCTCCAGGAAAACAATTG
GAAATGAATCACTAA

Further information on JAC1 can be obtained from the URL address http://db.yeastgenome.org/cgi-bin/singlepageformat?sgdid=S0000002986

It will be appreciated that, by “JAC1”, we include fragments or variants thereof having equivalent JAC1-like activity.

JID1 is another S. cerevisiae helper protein of interest for the present invention. It is one of several homologs of the bacterial chaperone DnaJ, and is located in the mitochondria. A published protein sequence for the protein Jid1p is as follows:

MLHHKFVYPFLFKWHLSCVEKCPPQITFIAKYATANDKNGNRKLTIRDEQ
WPELADPTPYDIFGIPKAGSGNPKLDKKSLKKKYHRYVKLYHPDHSDNIQ
IFSSEKVTNSDSKSPLLLTSSEKLHRFKVISQAYDILCDPKKKIVYDTTR
QGWTTSYSPRSNVNTENYQYAGSYGYHSNAQYEYWNAGTWEDANSMKN
ERIQENINPWTVIGIICGLAICIEGTALLAKIQESLSKAEFTHDESGLHL
IQSYTNYGLDTDKFSRLRRFLWFRTWGLYKSKEDLDREAKINEEMIRKLK
AAK

JID1 is encoded by a non-essential gene comprising an ORF that is 0.906 kbp in size and is located on chromosome XVI. A published nucleotide coding sequence of JID1 is as follows, although it will be appreciated that the sequence can be modified by degenerate substitutions to obtain alternative nucleotide sequences which encode an identical protein product:

ATGCTACACCATAAGTTCGTATACCCATTTTTATTCAAGTGGCACTTATCATGTGTAGAA
AAGTGTCCCCCACAAATCACTTTTATAGCTAAGTATGCTACAGCGAACGATAAAAATGGC
AATAGAAAACTTACGATAAGGGATGAACAATGGCCTGAGTTGGCAGATCCAACTCCCTAT
GATATTTTTGGCATTCCAAAGGCCGGATCTGGAAATCCTAAACTGGACAAGAAGTCGTTA
AAAAAAAAATATCATCGTTATGTAAAATTGTACCACCCTGACCATTCCGATAACATTCAA
ATATTTAGCTCAGAAAAGGTTACCAACAGTGATAGTAAATCACCGCTGCTGCTAACATCA
AGCGAAAAACTACATAGATTTAAAGTCATCTCTCAAGCATATGATATTCTTTGTGACCCA
AAGAAAAAGATCGTATATGACACAACGAGGCAAGGCTGGACCACATCGTATTCACCACGT
TCTAACGTTAATACTGAAAATTACCAATATGCCGGCTCTTATGGCTACCACTCTAACGCG
CAGTATGAATACTGGAACGCTGGGACTTGGGAAGACGCAAATAGCATGAAAAACGAAAGA
ATTCAAGAAAACATCAACCCATGGACCGTTATTGGCATAATTTGTGGCCTAGCTATATGC
ATCGAAGGGACTGCGTTGTTAGCCAAAATCCAGGAGTCTCTGAGCAAGGCCGAATTTACT
CATGACGAAAGTGGATTACATTTGATTCAGTCATACACGAATTATGGTCTTGATACTGAC
AAATTTTCCAGATTGAGGCGGTTCTTATGGTTTAGAACTTGGGGACTTTACAAGTCGAAA
GAGGATTTAGATAGAGAAGCCAAGATCAATGAAGAAATGATACGCAAACTGAAAGCAGCT
AAATGA

Further information on JID1 can be obtained from the URL address http://db.yeastgenome.org/cgi-bin/singlepageformat?sgdid=S000006265

It will be appreciated that, by “JID1”, we include fragments or variants thereof having equivalent JID1-like activity.

HLJ1 is another S. cerevisiae helper protein of interest for the present invention. It is one of several homologs of the bacterial chaperone DnaJ, and is located in the endoplasmic reticulum membrane. A published protein sequence for the protein Hlj1p is as follows:

MSFTEDQEKIALEILSKDKHEFYEILKVDRKATDSEIKKAYRKLAIKLHP
DKNSHPKAGEAFKVINRAFEVLSNEEKRSIYDRIGRDPDDRQMPSRGAAS
GFRGSAGGSPMGGGFEDMFFNSRFGGQRAGPPEDIFDFLFNAGGSPFGA
SPFGPSASTFSFGGPGGFRVYTNNRGGSPFMRQQPRSRQQQQQAEENA
VNSQLKNMLVLFIIFIVLPMIKDYLFS

HLJ1 is encoded by a non-essential gene comprising an ORF that is 0.675 kbp in size and is located on chromosome XIII. A published nucleotide coding sequence of HLJ1 is as follows, although it will be appreciated that the sequence can be modified by degenerate substitutions to obtain alternative nucleotide sequences which encode an identical protein product:

ATGTCTTTCACTGAGGATCAAGAAAAAATCGCGCTAGAAATACTGTCAAA
AGACAAGCATGAGTTTTACGAAATTTTGAAGGTAGATAGGAAAGCCACAG
ATAGTGAGATCAAGAAGGCATACAGAAAACTAGCAATCAAATTGCATCCT
GATAAAAACTCTCATCCAAAAGCGGGAGAAGCTTTCAAAGTAATTAATAG
GGCATTTGAAGTACTAAGCAATGAGGAAAAGCGCAGTATTTATGACAGGA
TAGGTAGGGATCCTGACGATAGACAAATGCCATCCAGAGGTGCTGCTTCA
GGGTTCCGAGGAAGTGCAGGTGGGTCTCCAATGGGTGGCGGATTTGAAGA
CATGTTTTTCAATTCACGTTTCGGTGGTCAAAGAGCTGGACCACCAGAGG
ACATATTCGACTTTTTGTTCAACGCAGGCGGCAGCCCATTCGGCGCTTCA
CCATTTGGGCCTTCTGCTTCCACTTTTTCATTTGGAGGCCCCGGTGGTTT
CAGAGTTTATACTAATAATCGTGGTGGCTCACCGTTCATGCGTCAACAAC
CCCGCTCAAGACAGCAGCAACAACAAGCAGAAGAAAATGCAGTGAATTCG
CAATTAAAAAATATGCTCGTTCTTTTCATCATCTTTATTGTTCTTCCTAT
GATTAAAGATTACCTGTTTAGTTAA

Further information HLJ1 can be obtained from the URL address http://db.yeastgenome.org/cgi-bin/singlepageformat?sgdid=S000004771

It will be appreciated that, by “HLJ1”, we include fragments or variants thereof having equivalent HLJ1-like activity.

ERJ5 is another S. cerevisiae helper protein of interest for the present invention. It is one of several homologs of the bacterial chaperone DnaJ, and is located in the endoplasmic reticulum. A published protein sequence for the protein Erj5p is as follows:

MNGYWKPALVVLGLVSLSYAFTTIETEIFQLQNEISTKYGPDMNFYKFLK
LPKLQNSSTKEITKNLRKLSKKYHPDKNPKYRKLYERLNLATQILSNSSN
RKIYDYYLQNGFPNYDFHKGGFYFSRMKPKTWFLLAFIWIVVNIGQYIIS
IIQYRSQRSRIENFISQCKQQDDTNGLGVKQLTFKQHEKDEGKSLVVRFS
DVYVVEPDGSETLISPDTLDKPSVKNCLFWRIPASVWNMTFGKSVGSAGK
EEIITDSKKYDGNQTKKGNKVKKGSAKKGQKKMELPNGKVIYSRK

ERJ5 is encoded by a non-essential gene comprising an ORF that is 0.888 kbp in size and is located on chromosome VI. A published nucleotide coding sequence of ERJ5 is as follows, although it will be appreciated that the sequence can be modified by degenerate substitutions to obtain alternative nucleotide sequences which encode an identical protein product:

ATGAACGGTTACTGGAAACCTGCGTTGGTTGTCCTGGGATTGGTATCTCT
ATCATATGCTTTTACCACCATTGAAACAGAAATTTTCCAATTACAAAATG
AAATAAGTACGAAATATGGCCCAGATATGAACTTCTACAAGTTCTTGAAG
TTACCTAAACTGCAGAATTCTAGTACAAAGGAGATTACAAAAAACTTAAG
AAAGCTATCCAAGAAGTACCATCCGGATAAGAACCCTAAATACCGTAAAT
TGTATGAAAGGTTAAACCTCGCTACTCAAATTCTTTCAAACAGCTCTAAT
CGTAAGATTTATGATTATTATCTACAGAATGGCTTTCCAAACTATGATTT
CCATAAGGGTGGTTTTTATTTTTCCAGAATGAAGCCTAAGACTTGGTTCC
TGCTGGCCTTTATTTGGATAGTCGTTAATATTGGGCAGTATATCATTTCT
ATTATTCAATATCGTTCTCAAAGATCAAGAATTGAAAACTTCATCAGTCA
GTGTAAACAACAGGATGATACCAATGGACTAGGCGTAAAACAACTAACGT
TTAAACAACATGAAAAGGATGAGGGTAAAAGTTTGGTTGTAAGGTTTAGC
GATGTCTATGTTGTAGAGCCTGATGGAAGTGAAACACTAATTTCGCCAGA
TACCTTGGATAAACCTTCAGTAAAGAACTGTTTGTTTTGGAGAATACCTG
CTTCGGTTTGGAACATGACGTTTGGCAAATCTGTTGGTAGCGCAGGAAAA
GAAGAAATAATAACGGATAGTAAAAAGTATGATGGTAACCAAACAAAAAA
GGGGAACAAAGTAAAAAAGGGTTCTGCAAAGAAAGGCCAAAAGAAAATGG
AATTGCCTAACGGTAAAGTGATCTATTCACGTAAATGA

Further information ERJ5 can be obtained from the URL address http://db.yeastgenome.org/cgi-bin/singlepageformat?sgdid=S000001937

It will be appreciated that, by “ERJ5”, we include fragments or variants thereof having equivalent ERJ5-like activity.

MGE1 is another S. cerevisiae helper protein of interest for the present invention and is also known as YGE1. It is one of several homologs of the bacterial GrpE and is located in the mitochondria. A published protein sequence for the protein Mge1p is as follows:

MRAFSAATVRATTRKSFIPMAPRTPFVTPSFTKNVGSMRRMRFYSDEAKS
EESKENNEDLTEEQSEIKKLESQLSAKTKEASELKDRLLRSVADFRNLQQ
VTKKDIQKAKDFALQKFAKDLLESVDNFGHALNAFKEEDLQKSKEISDLY
TGVRMTRDVFENTLRKHGIEKLDPLGEPFDPNKHEATFELPQPDKEPGTV
FHVQQLGFTLNDRVIRPAKVGIVKGEEN

MGE1 is encoded by an essential gene comprising an ORF that is 0.687 kbp in size and is located on chromosome XV. A published nucleotide coding sequence of MGE1 is as follows, although it will be appreciated that the sequence can be modified by degenerate substitutions to obtain alternative nucleotide sequences which encode an identical protein product:

ATGAGAGCTTTTTCAGCAGCCACCGTTAGGGCCACAACTAGGAAGTCGTT
CATCCCAATGGCACCAAGAACTCCTTTTGTGACTCCATCATTTACAAAGA
ATGTAGGCTCAATGAGAAGAATGAGATTTTATTCTGATGAAGCCAAAAGT
GAAGAATCCAAAGAAAACAATGAAGATTTGACTGAAGAGCAATCAGAAAT
CAAGAAATTAGAGAGCCAGTTAAGCGCGAAGACTAAAGAAGCTTCTGAAC
TCAAGGACAGATTATTAAGATCTGTGGCAGATTTCAGAAATTTACAACAA
GTCACAAAGAAGGATATTCAGAAAGCTAAGGACTTTGCTTTACAGAAGTT
TGCAAAGGATTTATTGGAATCTGTAGATAACTTTGGTCATGCTTTGAATG
CTTTTAAAGAGGAAGACTTACAAAAGTCCAAGGAAATTAGTGATTTGTAT
ACAGGGGTTAGAATGACAAGAGATGTTTTTGAAAACACCCTAAGAAAGCA
CGGTATTGAAAAATTAGACCCATTGGGAGAACCATTTGATCCAAATAAAC
ACGAAGCAACGTTCGAGTTGCCACAACCTGATAAGGAACCGGGTACTGTT
TTCCATGTACAACAATTAGGTTTCACCTTGAATGACAGAGTTATCAGACC
AGCAAAAGTCGGAATTGTTAAGGGCGAAGAGAACTAA

Further information MGE1 can be obtained from the URL address http://db.yeastgenome.org/cgi-bin/singlepageformat?sgdid=S000005758

It will be appreciated that, by “MGE1”, we include fragments or variants thereof having equivalent MGE1-like activity.

FES1 is another S. cerevisiae helper protein of interest for the present invention. It is one of several homologs of the bacterial GrpE and is located in the cytoplasm. A published protein sequence for the protein Fes1p is as follows:

MEKLLQWSIANSQGDKEAMARAGQPDPKLLQQLFGGGGPDDPTLMKESMA
VIMNPEVDLETKLVAFDNFEMLIENLDNANNIENLKLWEPLLDVLVQTKD
EELRAAALSIIGTAVQNNLDSQNNFMKYDNGLRSLIEIASDKTKPLDVRT
KAFYALSNLIRNHKDISEKFFKLNGLDCIAPVLSDNTAKPKLKMRAIALL
TAYLSSVKIDENIISVLRKDGVIESTIECLSDESNLNIIDRVLSFLSHLI
SSGIKFNEQELHKLNEGYKHIEPLKDRLNEDDYLAVKYVL

FES1 is encoded by a non-essential gene comprising an ORF that is 0.873 kbp in size and is located on chromosome II. A published nucleotide coding sequence of FES1 is as follows, although it will be appreciated that the sequence can be modified by degenerate substitutions to obtain alternative nucleotide sequences which encode an identical protein product:

ATGGAAAAGCTATTACAGTGGTCTATTGCGAATTCTCAAGGGGACAAAGA
AGCTATGGCTAGGGCCGGCCAACCTGATCCTAAATTGCTACAGCAGTTAT
TCGGTGGTGGTGGTCCTGACGATCCAACCTTAATGAAAGAATCCATGGCT
GTTATTATGAATCCGGAGGTTGACTTAGAAACAAAACTCGTTGCATTTGA
CAACTTTGAAATGTTGATTGAGAACTTAGATAATGCTAATAATATCGAAA
ATTTAAAACTGTGGGAGCCATTGTTGGATGTTCTTGTTCAGACGAAGGAT
GAAGAACTACGTGCTGCTGCTTTATCCATTATTGGAACGGCTGTGCAAAA
CAACTTGGATTCGCAAAATAATTTCATGAAATACGACAATGGTCTGCGAA
GCCTTATCGAAATAGCTAGTGACAAGACAAAGCCACTCGACGTGAGAACA
AAAGCTTTTTACGCACTATCTAATCTAATAAGAAACCACAAAGATATCTC
AGAAAAGTTTTTCAAATTAAATGGGCTCGACTGCATAGCACCTGTATTAA
GTGATAACACCGCCAAACCAAAACTGAAAATGAGAGCCATTGCCTTATTG
ACCGCATATTTGTCATCTGTTAAGATTGATGAAAATATAATCAGTGTGCT
GAGAAAGGATGGAGTAATTGAAAGTACGATTGAGTGCTTGTCTGACGAGA
GTAACTTGAACATCATAGATAGAGTTCTGTCTTTTCTCTCTCACCTGATA
TCTTCCGGAATAAAATTTAATGAACAGGAATTGCACAAATTGAACGAAGG
TTACAAACATATCGAGCCTCTAAAGGACAGACTTAATGAAGACGATTATT
TAGCCGTAAAGTATGTATTATGA

Further information FES1 can be obtained from the URL address http://db.yeastgenome.org/cgi-bin/singlepageformat?sgdid=S000000305

It will be appreciated that, by “FES1”, we include fragments or variants thereof having equivalent FES1-like activity.

Variants and fragments of the above JEM1, LHS1, SCJ1, KAR2, SIL1, FKB2, SSA1, SSA2, SSA3, SSA4, SSE1, SSE2, SSB1, SSB2, ECM10, MDJ1, MDJ2, ERO1, ERV2, EUG1, MPD1, MPD2, EPS1, PDI1, DER1, DER3, HRD3, UBC7, DOA4, HAC1, SEC63, YDJ1, XDJ1, APJ1, SIS1, DJP1, ZUO1, SWA2, JJJ1, JJJ2, JJJ3, CAJ1, CWC23, PAM18, JAC1, JID1, HLJ1, ERJ5, MGE1 and FES1 proteins and encoding polynucleotide sequences, and variants of other naturally occurring JEM1, LHS1, SCJ1, KAR2, SIL1, FKB2, SSA1, SSA2, SSA3, SSA4, SSE1, SSE2, SSB1, SSB2, ECM10, MDJ1, MDJ2, ERO1, ERV2, EUG1, MPD1, MPD2, EPS1, PDI1, DER1, DER3, HRD3, UBC7, DOA4, HAC1, SEC63, YDJ1, XDJ1, APJ1, SIS1, DJP1, ZUO1, SWA2, JJJ1, JJJ2, JJJ3, CAJ1, CWC23, PAM18, JAC1, JID1, HLJ1, ERJ5, MGE1 and FES1 proteins and encoding polynucleotide sequences are also included in the present invention.

A “variant”, in the context of a JEM1, LHS1, SCJ1, KAR2, SIL1, FKB2, SSA1, SSA2, SSA3, SSA4, SSE1, SSE2, SSB1, SSB2, ECM10, MDJ1, MDJ2, ERV2, EUG1, MPD1, MPD2, EPS1, PDI1, DER1, DER3, HRD3, UBC7, DOA4, HAC1, SEC63, YDJ1, XDJ1, APJ1, SIS1, DJP1, ZUO1, SWA2, JJJ1, JJJ2, JJJ3, CAJ1, CWC23, PAM18, JAC1, JID1, HLJ1, ERJ5, MGE1 or FES1 protein, refers to a protein having a sequence as defined above by the present application wherein at one or more positions there have been amino acid insertions, deletions, or substitutions, either conservative or non-conservative, provided that such changes result in a protein whose basic properties, for example enzymatic activity (type of and specific activity), thermostability, activity in a certain pH-range (pH-stability) have not significantly been changed. “Significantly” in this context means that one skilled in the art would say that the properties of the variant may still be different but would not be unobvious over the ones of the original protein.

By “conservative substitutions” is intended combinations such as Val, Ile, Leu, Ala, Met; Asp, Glu; Asn, Gln; Ser, Thr, Gly, Ala; Lys, Arg, His; and Phe, Tyr, Trp. Preferred conservative substitutions include Gly, Ala; Val, Ile, Leu; Asp, Glu; Asn, Gln; Ser, Thr; Lys, Arg; and Phe, Tyr.

A “variant” typically has at least 25%, at least 50%, at least 60% or at least 70%, preferably at least 80%, more preferably at least 90%, even more preferably at least 95%, yet more preferably at least 99%, most preferably at least 99.5% sequence identity to the polypeptide from which it is derived.

The percent sequence identity between two polypeptides may be determined using suitable computer programs, as discussed below. Such variants may be natural or made using the methods of protein engineering and site-directed mutagenesis as are well known in the art.

A “fragment”, in the context of JEM1, LHS1, SCJ1, KAR2, SIL1, FKB2, SSA1, SSA2, SSA3, SSA4, SSE1, SSE2, SSB1, SSB2, ECM10, MDJ1, MDJ2, ERO1, ERV2, EUG1, MPD1, MPD2, EPS1, PDI1, DER1, DER3, HRD3, UBC7, DOA4, HAC1, SEC63, YDJ1, XDJ1, APJ1, SIS1, DJP1, ZUO1, SWA2, JJJ1, JJJ2, JJJ3, CAJ1, CWC23, PAM18, JAC1, JID1, HLJ1, ERJ5, MGE1 and FES1 proteins, refers to a protein wherein at one or more positions there have been deletions. Thus the fragment may comprise at most 5, 10, 20, 30, 40 or 50%, typically up to 60%, more typically up to 70%, preferably up to 80%, more preferably up to 90%, even more preferably up to 95%, yet more preferably up to 99% of the complete sequence of the full mature protein as defined above. Particularly preferred fragments of a protein comprise one or more whole domains of the desired protein.

A fragment or variant of a JEM1, LHS1, SCJ1, KAR2, SIL1, FKB2, SSA1, SSA2, SSA3, SSA4, SSE1, SSE2, SSB1, SSB2, ECM10, MDJ1, MDJ2, ERO1, ERV2, EUG1, MPD1, MPD2, EPS1, PDI1, DER1, DER3, HRD3, UBC7, DOA4, HAC1, SEC63, YDJ1, XDJ1, APJ1, SIS1, DJP1, ZUO1, SWA2, JJJ1, JJJ2, JJJ3, CAJ1, CWC23, PAM18, JAC1, JID1, HLJ1, ERJ5, MGE1 or FES1 protein may be a protein that, when expressed recombinantly in a host cell, can complement the deletion of the same endogenously encoded gene in the host cell, such as S. cerevisiae, and may or may not, for example, be a naturally occurring homolog of the protein upon which it is based, such as a homolog encoded by another organism, such as another yeast or other fungi, or another eukaryote such as a human or other vertebrate, or animal or by a plant.

A fragment or a variant of a polynucleotide encoding a JEM1, LHS1, SCJ1, KAR2, SIL1, FKB2, SSA1, SSA2, SSA3, SSA4, SSE1, SSE2, SSB1, SSB2, ECM10, MDJ1, MDJ2, ERO1, ERV2, EUG1, MPD1, MPD2, EPS1, PDI1, DER1, DER3, HRD3, UBC7, DOA4, HAC1, SEC63, YDJ1, XDJ1, APJ1, SIS1, DJP1, ZUO1, SWA2, JJJ1, JJJ2, JJJ3, CAJ1, CWC23, PAM18, JAC1, JID1, HLJ1, ERJ5, MGE1 or FES1 protein may be a polynucleotide that comprises a sequence that encodes a fragment or variant of the protein as defined above.

The present invention will now be exemplified with reference to the following non-limiting examples and figures.

### BRIEF DESCRIPTION OF FIGURES

FIGS. 1 to 9, 11 to 16, 21, 23-25 and 28 show various plasmid maps as described in the following examples.

FIG. 10 shows analysis of HAC1 splicing at log phase by qRT-PCR in the strain AH22 (ura3) [pAYE329]. Helper protein overexpression plasmids are shown on the x-axis. Data are normalised to ACT1 transcript levels and presented as fold changes from AH22 (ura3) [pAYE329, YCplac33]. All values shown represent duplicate analysis of mRNA levels from single experimental cultures.

FIG. 17 shows SDS-PAGE gels for quantification of rHA production in overexpression strains. Sample labels shown indicate overexpression plasmids transformed into the strain AH22 (ura3) [pAYE329]. Duplicate samples represent two independent shake flasks from the same transformant.

FIG. 18 shows quantification of main rHA band in transformed and control strains, by analysis of SDS-PAGE gel of FIG. 17 using densitometry. Values are normalised (based on culture optical density readings) to account for different growth rates observed between strains.

FIG. 19 shows quantification of main rHA band in transformed and control strains, by analysis of SDS-PAGE gel of FIG. 17 using densitometry, expressed as a percentage of determined rHA production by the negative control YCplac33. Values are normalised (based on culture optical density readings) to account for different growth rates observed between strains.

FIG. 20 shows quantification of rHA fragments relative to total rHA, by analysis of SDS-PAGE gel of FIG. 17 using densitometry, expressed as a percentage of detected rHA fragments relative to total rHA levels detected (total rHA=full length rHA+degradation products). Values are normalised (based on culture optical density readings) to account for different growth rates observed between strains.

FIG. 22 shows a comparison of recombinant transferrin titres by rocket immunoelectrophoresis. A=Control Strain [pDB3213]; B=Control Strain (ura3). [pTPC17 pDB3213]. Duplicate 10 mL shake flasks cultures were inoculated with yeast and incubated with shaking at 200 rpm for 4-days at 30° C. 5 μL culture supernatant loaded per well of a rocket immunoelectrophoresis gel. Plasma Tf standards concentrations are in μg/mL. 20 μL goat anti-Tf/50 mL agarose. Precipin was stained with Coomassie blue.

FIG. 26 shows the effect of LHS1, JEM1 and SIL1 co-expression on rHA production, when rHA is fused to different leader sequences. Two separate transformants for each strain were inoculated into 50 mL shake flasks containing 10 mL BMMD and incubated with shaking at 200 rpm for 4-days at 30° C. 20 μL of culture supernatant was loaded per well of a 4-12% SDS-PAGE gel and run for 50 mins in MOPS buffer. Gel A shows the results obtained with plasmid pDB2244, which encodes a HSA/MFα-1 fusion leader sequence (A=AH22 (ura3) [pDB2244 YCplac33]; B=AH22 (ura3) [pDB2244 pTPC17]). Gel B shows the results obtained with plasmid pDB2286, which encodes an invertase leader sequence (C=AH22 (ura3) [pDB2286 YCplac33]; D=AH22 (ura3) [pDB2286 pTPC17]). Gel C shows the results obtained with plasmid pDB2287, which encodes the MFα-1 leader sequence (E=AH22 (ura3) [pDB2287 YCplac33]; F=AH22 (ura3) [pDB2287 pTPC17]).

FIG. 26, part D, shows densitometric quantification of rHA secretion. Gels shown in FIG. 26 A-C were analysed by densitometry and comparison to rHA standard curves. Data presented above represents quantification of single rHA bands. For each strain two transformants were analysed (samples A and B in FIG. 26D).

FIG. 27 shows the DNA sequence of the human GM-CSF cDNA with an incorporated N-terminal Met codon.

FIG. 29 A shows an SDS-PAGE gel for quantification of GM-CSF production. Lanes 2-5 show GM-CSF production in the control strain (ura3) [pDB2109 YCplac33]. Lanes 6-9 show GM-CSF production in the control strain (ura3) [pDB2109 pTPC17.

FIG. 29 B shows the results of densitometric analysis of the SDS-PAGE gel shown in FIG. 29 A, as further given in Table 9, below.

### EXAMPLE 1

A strain of S. cerevisiae that possesses increased production of a recombinant protein was produced by the following methodology.

Strains. The S. cerevisiae strain used was a histidine revertant of AH22 (cir^{o} a leu2-3 leu2-112 his4 canR). AH22 is further described in Mead at al, 1986, Mol. Gen. Genet., 205, 417-421. A polynucleotide encoding a recombinant heterologous protein expression cassette was introduced by S. cerevisiae transformation performed according to Ito, H., at al. (Transformation of intact yeast cells treated with alkali cations. J. Bacterial. 153, 163-168, (1983)).

Media. Yeast strains were grown in rich broth medium, YEP (1% yeast extract 2% w/v Bactopeptone).

Protein assays. Yeast cells were grown in 10 ml cultures for 72 hours to a density of 5×10⁷ cells/mL at 30° C. in YEP 2% (w/v) sucrose. In order to analyse the soluble heterologous protein fraction of yeast, cells were harvested by centrifugation and disrupted in phosphate buffered saline by vortexing with 40 mesh glass beads. The soluble fraction was collected as the supernatant of a 10,000×g centrifugation. The fraction was assayed for the presence of heterologous protein by polyacrylamide gel electrophoresis and Western blot, using appropriate commercially available antibodies.

Mutagenesis. Yeast cells to be mutated were grown in 100 ml defined medium (0.65% (w/v) YNB; 2% (w/v) sucrose; Na₂HPO₄/citric acid pH 6.5) to OD₆₅₀=0.5. Cells were harvested by centrifugation and resuspended in 100 ml defined medium. To 2 ml of washed cells was added 10 microlitres, 20 microlitres, 40 microlitres, 80 microlitres or 160 microlitres of the mutagen stock solution. The cells were then incubated at 30° C., 200 rpm for 30 rain. One ml of mutated cells was washed twice with 1 ml sterile distilled water and finally resuspended in 1 ml YEP. The percentage of cells that survived the mutagenic treatment was assessed by spreading an aliquot of each mutagenic reaction onto YEP, 2% (w/v) sucrose plates. Mutagen stock solutions were prepared as follows. N-methyl-N-nitro-N-nitrosoguanidine (NTG) was dissolved in ethanol at 5 mg/mL; 4 nitroquinoline N-oxide (NQO) was resuspended in acetone at 10 mg/mL and then diluted 1 in 100 to 0.1 mg/mL with K₂HPO₄/KH₂PO₄ (pH 7.0); 1,2,7,8-diepoxyoctane (DEO) and ethyl methanesulphonate (EMS) were both supplied as liquids (Sigma) and were used without dilution.

After mutagenesis, a S. cerevisiae strain was identified with a higher level of production of a recombinant protein, compared to its ancestral strain (data not shown).

### EXAMPLE 2

The expression of genes in the strain identified in Example 1 was compared to the expression of genes in the ancestral strain from which it was derived (i.e. the ancestral strain displays lower levels of production of a recombinant protein).

The comparison was made by using microarray analysis. Yeast cells to be analysed were grown in 100 ml defined medium (0.65% (w/v) YNB; 2% (w/v) dextrose; Na₂HPO₄/citric acid pH 6.5) to OD₆₀₀=2.0. The cells were immediately harvested by centrifugation and frozen by immersion in liquid nitrogen. RNA suitable for microarray analysis was prepared by disruption of the cells using a micro dismembrator (Braun Melsungen, Germany) all as described by Jones et al, 2003, Physiol. Genomics, 16, 107-118. cDNA synthesis, labelling, hybridisation to high-density oligonucleotide arrays (Affymetrix-Yeast S98) and scanning were carried out as described by protocols provided by the manufacturer (Affymetrix Inc, USA). The subsequent data was analysed using the MAS 5.1 and DTM 3.0 software programs (Affymetrix Inc, USA).

Genes identified as being up-regulated in the strain identified in Example 1, compared to the ancestral strain, include—

TABLE 1

| Gene | Fold change | Gene | Fold change |
|---|---|---|---|
| JEM1 | 2.63 | EUG1 | 3.68 |
| LHS1 | 2.40 | MPD1 | 2.37 |
| SCJ1 | 1.81 | MPD2 | 1.51 |
| KAR2 | 1.24 | EPS1 | 1.10 |
| SIL1 | 4.5 | PDI1 | 1.22 |
| FKB2 | 1.62 | DER1 | 2.64 |
| SSA3 | 2.61 | DER3 | 1.67 |
| SSA4 | 1.83 | HRD3 | 1.82 |
| SSE2 | 2.31 | UBC7 | 1.33 |
| ECM10 | 5.65 | DOA4 | 1.91 |
| ERO1 | 2.66 | HAC1 | 2.05 |
| ERV2 | 1.73 | | |

It will be recognised that none of SSA1, SSA2, SSE1, SSB1, SSB2, MDJ1 or MDJ2 were identified as being over-expressed in the strain identified in Example 1. However, these helper proteins have been included in the present invention as a result of their functional association to the helper proteins whose genes have been identified as being upregulated in the strain isolated in Example 1. For example, the genes encoding SSA3, SSA4 and SSB2 have all been identified as being over-expressed; SSA1, SSA2, SSE1, SSB1 and SSB2 are functional equivalents of these helper proteins and so it is anticipated that over-expression of the genes encoding any of SSA1, SSA2, SSE1, SSB1 or SSB2 would cause the same phenotype as the over-expression of the genes encoding any of SSA3, SSA4 or SSB2. Similarly the gene encoding ECM10 has been identified as being over-expressed; MDJ1 and MDJ2 are functional equivalents of ECM10 and so it is anticipated that the over-expression of either of the genes encoding MDJ1 or MDJ2 would cause the same phenotype as the over-expression of the gene encoding ECM10.

### EXAMPLE 3

The example describes the vector construction and yeast transformation for the overexpression of the representative helper proteins LHS1, SLS1, JEM1 and SCJ1.

TABLE 2
Primers used

| | |
|---|---|
| Primer name | Sequence (5′-3′) |

| | |
|---|---|
| HO 5′ ForNotIBbsI | GCATGCGGCCGCCCGAAGACCCTACACAGGGCTTAAGGGC |
| HO 5′ RevBsiWIMluI | CCACGCGTCGTACGGGATTGCTGCTTATGAGGATA |
| HO 3′ ForMluIEcoRI | ACGCGTGAATTCAAAAAGGGAACCCGTATATTTCAGC |
| HO 3′ RevBbsIClaI | TATCGATAGTCTTCCTAATATACACATTTTAGCAGATGC |
| pBST HO Poly For | GCATGCATACGCGTCACGCATGTGCCTCAGCGGCCGGCCGGCGCCGGGCCCC |
| | GGACCGCCTGCAGGCTCGAGTTAATTAAGTTTAAACGAATTCGCATGCAT |
| pBST HO Poly Rev | ATGCATGCGAATTCGTTTAAACTTAATTAACTCGAGCCTGCAGGCGGTCCGG |
| | GGCCCGGCGCCGGCCGGCCGCTGAGGCACATGCGTGACGCGTATGCATGC |
| Ycplac33 Poly For | CTAGATTGGATCCCTAGTCTAGGTTTAAACTAGCGATTCACCTAGGTGCTAG |
| | GAATTCTAGC |
| Ycplac33 Poly Rev | GCTAGAATTCCTAGCACCTAGGTGAATCGCTAGTTTAAACCTAGACTAGGGA |
| | TCCAATCTAG |
| LHS1forOverlap | CACAATATTTCAAGCTATACCAAGCATACAATCAACTATCTCATATA |
| | CAATGCGAAACGTTTTAAGGCT |
| LHS1revBbvCI | GCATGCTGAGGGTGCCACTATAATATTAATGTGC |
| SLS1forOverlap | CACCAACACACACAAAAAACAGTACTTCACTAAATTTACACACAAA |
| | ACAAAATGGTCCGGATTCTTCCCAT |
| SLS1revNarI | GCATGGCGCCCCACGGCAGGGCAGTTGGCAC |
| JEM1forOverlap | CAGATCATCAAGGAAGTAATTATCTACTTTTTACAACAAATATAAAA |
| | CAATGATACTGATCTCGGGATAC |
| JEM1revRsrII | CGATCGGTCCGAGGGAAATAAGGCAGATCAAAG |
| SCJ1forOverlap | CACGCTTACTGCTTTTTTCTTCCCAAGATCGAAAATTTACTGAATTAA |
| | CAATGATTCCAAAATTATATATAC |
| SCJ1revXhoI | GCATCTCGAGGACTTTGAGACCTGTGATC |
| ADH1promForAleI | CGATCACCGATGTGGTTGTTTCCGGGTGTACAATATGG |
| ADH1promRevOverlap | CCTATAGCAACAAAAGCTGTTAAAAATAAAAGCCTTAAAACGTTTCG |
| | CATTGTATATGAGATAGTTGATTG |
| PGK1promForPspOMI | GCATGGGCCCAGATTCCTGACTTCAACTCAAG |
| PGK1promRevOverlap | GGCAAAATAACGCTATACACTAAAAGACAGTATCCCGAGATCAGTAT |
| | CATTGTTTTATATTTGTTGTAAAAAC |
| TDH1promForFseI | GCATGGCCGGCCACCATATGGAGGATAAGTTGG |
| TDH1promRevOverlap | CTAATTTCGAAGATAGGGCGCTCAAAATTATGGGAAGAATCCGGACC |
| | ATTTTGTTTTGTGTGTTTTAAATC |
| TEF1promForSbfI | CGGTAGTACCTGCAGGAAGCAACAGGCGCGTTGGAC |
| TEF1promRevOverlap | GGCAACAACAATAAAGATAGTATCAAATGTATATATAATTTTGGAAT |
| | CATTTTGTAATTAAAACTTAGATTAGATTGC |
| URA3forPac1 | CTAGAGTTAATTAAGTTTCAATTCAATTCATC |
| URA3revPme1 | GCCTGAGTTTAAACGTTTTCTTTCCAATTTTT |

pBST HO regions: HO regions were amplified by PCR from BY4741 (Brachmann et al., 1998, Yeast, 30; 14(2):115-32) genomic DNA using the primers shown in Table 2. Fast Start High Fidelity PCR system (Roche) was used with the conditions as recommended: 50 μL final volume containing 0.2 mM dNTPs, 1.8 mM MgCl₂, 0.4 μM forward and reverse primers, 100 ng template genomic DNA, 2.5 U polymerase and H₂O to volume. Cycling conditions: 95° C. for 2 mins followed by 35 cycles of 95° C. 30 s, 60° C. 30 s, 72° C. 1 min and 72° C. 7 reins for final elongation.

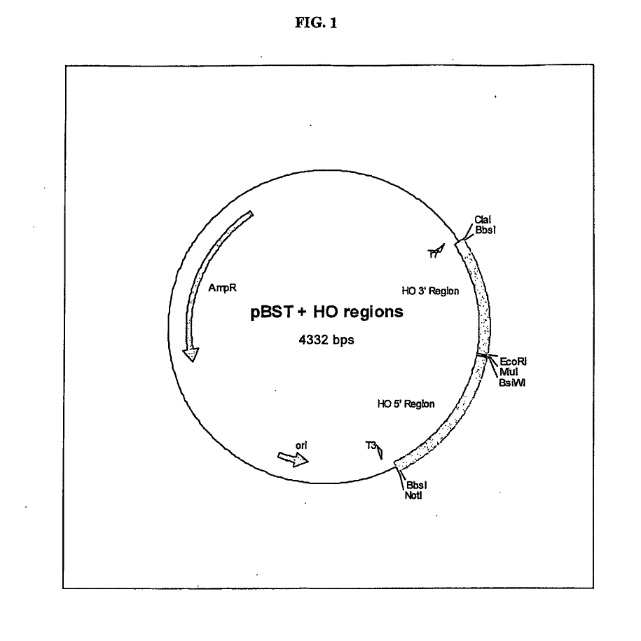

Fragments were gel extracted from a 1% (w/v) agarose TAE gel using the GeneClean III kit (Q-bio Gene). Purified DNA was digested with the appropriate enzymes, NotI and MluI for HO 5′ region, MluI and ClaI for HO 3′ region. pBST+ (WO99/00504) was digested with NotI and ClaI. Fragments were purified as above. A three way ligation was performed using a Rapid Ligation Kit (Roche) as per manufacturers instructions. Ligations were transformed into the E. coli strain DH5α. Diagnostic restriction digests were performed on mini-prep DNA to confirm the ligation was successful. The plasmid map is shown in FIG. 1.

Polylinkers: To facilitate the cloning of the helper genes a polynucleotide linkers were incorporated into pBST+HO regions (FIG. 1) and into YCplac33 (Gietz and Sugino, 1988, Gene, 74, 527-534).

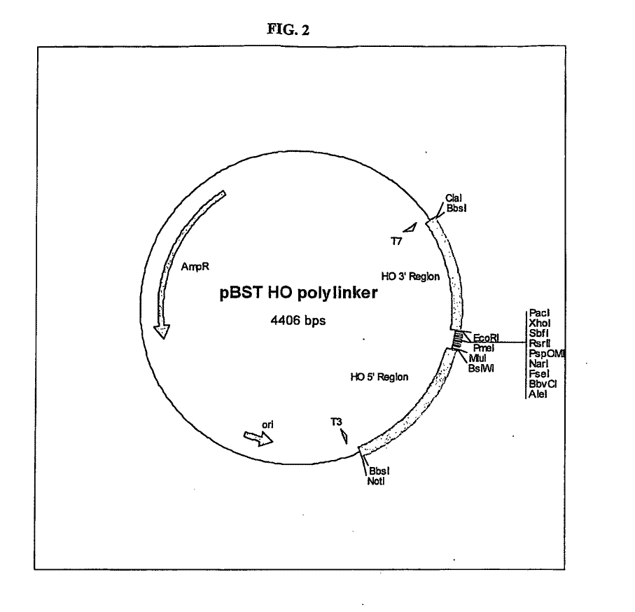

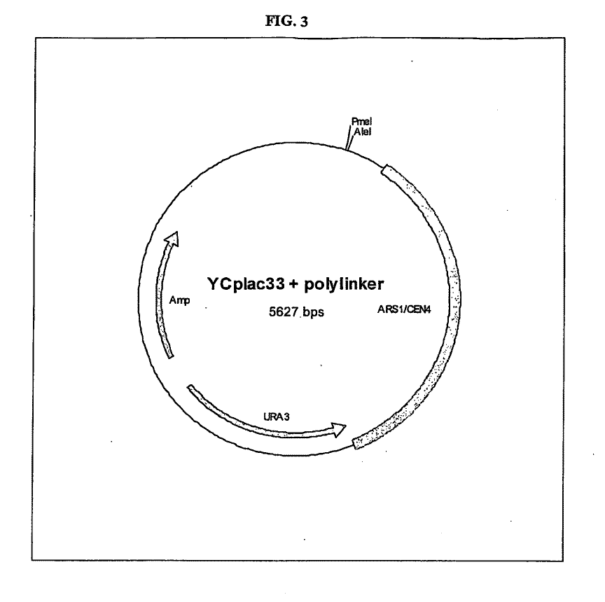

Complementary single stranded oligonucleotides were annealed as follows: 1 μL of a 100 μM solution of each oligo (Poly For and Poly Rev, Table 2) was added into a 50 μL total volume containing 10× restriction buffer (Roche Buffer H for pBST HO polylinker, Buffer B for YCplac33 polylinker). Samples were placed into a PCR machine and heated to 98° C. for 4 mins. Samples were then held for 1 min with the temperature dropping 1° C. every cycle down to 30° C. The annealed polylinkers were then digested by addition of the appropriate restriction enzyme (MluI, EcoRI for pBST HO polylinker, BamHI, EcoRI for YCplac33 polylinker). Digested polylinkers were gel extracted as previously and ligated into the corresponding vector digests. Incorporation of polylinkers was confirmed by linearising plasmids with all restriction sites present in polylinkers. Vectors produced are shown as FIGS. 2 and 3 respectively.

Production of promoter/open reading frame constructs: All four open reading frames (ORFs) and promoters were amplified by PCR, from the genomic DNA of an AH22 derivative, using Vent polymerase (NEB). Reactions were setup as per manufacturers instructions with an annealing temperature of 50° C. All fragments were gel extracted and resuspended in 5 μL of water. 1 μL was run on gel to check fragment presence and quantity.

Promoters and ORFs were joined according to the method of Shevchuk et al. (Nucleic Acids Res., 2004, 32(2), e19.). 100 ng of ORF and an equimolar amount of promoter was used in the first PCR stage. 10 μL from this was used in the second PCR stage. Primers were added to a final concentration of 0.4 μM.

Second stage PCRs were run on a 1% (w/v) agarose TAE gel and bands extracted of the expected size (promoter+ORF length). Extracted fragments were A-tailed using Fast Start High Fidelity polymerase (Roche) and cloned into the Topo pCR2.1 vector (Invitrogen). Plasmid DNA was restriction digested to confirm the correct insert and subsequently sequenced.

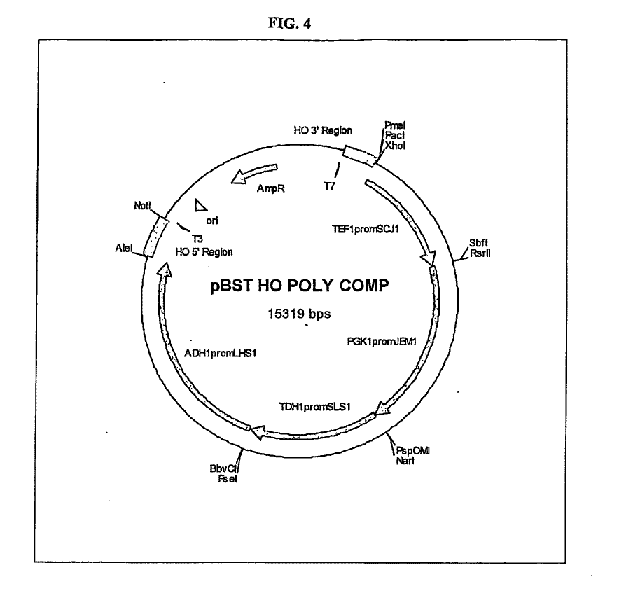

Assembly of overexpression constructs: Restriction digests were performed to release promoter/ORF constructs from Topo pCR2.1 vectors. Fragments were gel extracted and ligated into the pBST HO polylinker vector, digested accordingly. In the first instance, constructs were produced containing each individual promoter/ORF and containing all four. This required subsequent rounds of plasmid transformation, digestion and ligation. The vector containing all four promoter/ORFs is shown in FIG. 4.

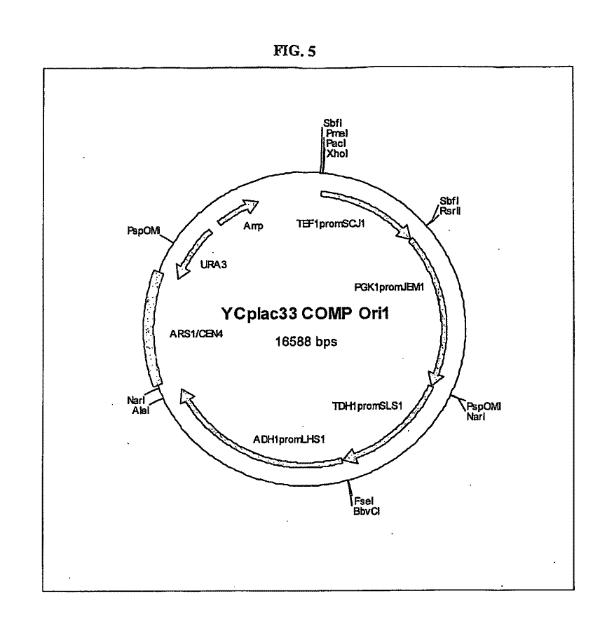

For insertion of promoter/ORF constructs into the centromeric vector, YCplac33 polylinker, a PmeI/AleI digest was performed on pBST HO POLY (FIG. 4) containing the required promoter/ORFs, and YCplac33 polylinker. The fragment released from pBST HO POLY was ligated with the digested YCplac33 polylinker vector. The vector containing all four promoter/ORFs is shown in FIG. 5.

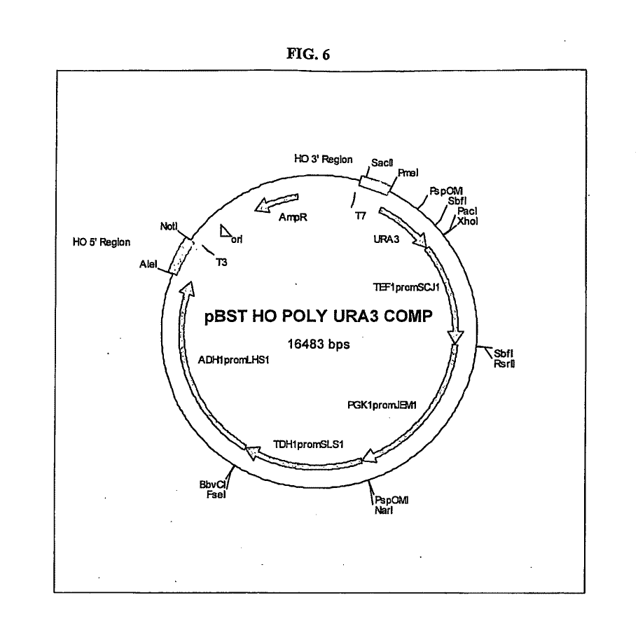

Insertion of URA3 marker into pBST HO POLY: The URA3 marker was amplified by PCR from the vector YCp50 (Rose et al., 1987, Gene, 60, 237-243) using Fast Start High Fidelity polymerase (Roche) with an annealing temperature of 50° C. The fragment was gel extracted, digested with PacI/PmeI and ligated into each pBST HO POLY vector containing the required promoter/ORFs (also PacI/PmeI digested). It is important the URA3 fragment be introduced last as it contains sites for restriction enzymes used elsewhere in construction of the plasmid. The vector produced containing all four promoter/ORFs is shown in FIG. 6.

Chromosomal integration: The helper gene constructs were integrated into the genome of a S. cerevisiae host cell as follows. The vector pBST HO POLY URA3 COMP (FIG. 6) was digested with NotI and SacII. Approximately 2-3 μg of the required fragment was gel extracted and used to transform a ura3 derivative of AH22 [pAYE329] using a yeast transformation kit (Sigma). Transformations were plated onto minimal media and incubated at 30° C. until colonies appeared. The construction of plasmid pAYE329 is described in Sleep et al., 1990, Gene, 101, 89-96. A ura3 auxotrophic mutant of the AH22 derivative was created by 5-fluoro-orotic acid selection as described by Boeke et al, 1987, Methods Enzymol., 154, 164-175.

Alternatively, the helper gene constructs may be introduced on a centromeric vector. For the YCplac33 based-vectors, 500 ng of plasmid DNA may be used to transform a S. cerevisiae host cell as above.

### EXAMPLE 4

This example describes a modified protocol for vector construction and yeast transformation for the overexpression of the representative helper proteins LHS1, SIL1; JEM1 and SCJ1.

TABLE 3
Primers used

| Primer | | Sequence (5′-3′) - Regions underlined indicate restriction enzyme |
|---|---|---|
| name | Product | cleavage sites and are followed by the name of the cleaving enzyme. |
| A01 | H0 5′ region | GCATGCGGCCGC(NotI)CCGAAGAC(BbsI)CCTACACAGGGCTTAAGGGC |
| A02 | | CCACGCGT(MluI)CGTACG(BsiWI)GGATTGCTGCTTATGAGGATA |
| A03 | HO 3′ region | ACGCGT(MluI)GAATTC(EcoRI)AAAAAGGGAACCCGTATATTTCAGC |
| A04 | | TATCGAT(ClaI)AGTCTTC(BbsI)CTAATATACACATTTTAGCAGATGC |
| A05 | pTPA02 | GCATGCATACGCGT(MluI)CACGCATGTGCCTCAGC(BbvCI)GGCCGGCC |
| | poly-linker | (FseI)GGCGCC(NarI)GGGCCC(PspOMI)CGGACCG(RsrII)CCTGCAGG(SbfI) |
| | | CTCGAG(XhoI)TTAATTAA(PacI)GTTTAAAC(PmeI)GAATTC(EcoRI)GCA |
| | | TGCAT |
| A06 | | ATGCATGCGAATTC(EcoRI)GTTTAAAC(PmeI)TTAATTAA(PacI)CTCGA |
| | | G(XhoI)CCTGCAGG(SbfI)CGGTCCG(RsrII)GGGCCC(PspOMI)GGCGCC(NarI) |
| | | GGCCGGCC(FseI)GCTGAGG(BbvCI)CACATGCGTGACGCGT(MluI)AT |
| | | GCATGC |
| A07 | ACT1 | CTAGGTAACTTAATTAA(PacI)GGGTAAGCTGCCACAGCA |
| A08 | promoter | CTACGTACTCTAGA(XbaI)TGTTAATTCAGTAAATTTTC |
| A09 | ACT1 | CTAGACTCTAGA(XbaI)TCTCTGCTTTTGTGCGCG |
| A10 | terminator | CATGCTACGTTTAAAC(PmeI)GATGATCATATGATACAC |
| A11 | URA3 region | CTAGAGTTAATTAA(PacI)GTTTCAATTCAATTCATC |
| A12 | | GCCTGAGTTTAAAC(PmeI)GTTTTCTTTCCAATTTTT |
| A13 | pTPA05 | CTAGATTGGATCCCTAGTCTAGGTTTAAACTAGCGATTCACCTAGGTG |
| | poly-linker | (AleI)CTAGGAATTCTAGC |
| A14 | | GCTAGAATTCCTAGCACCTAGGTG(AleI)AATCGCTAGTTTAAACCTAG |
| | | ACTAGGGATCCAATCTAG |
| C01 | LHS1 ORF/ | CACAATATTTCAAGCTATACCAAGCATACAATCAACTATCTCATATAC |
| | terminator | AATGCGAAACGTTTTAAGGCT |
| C02 | | GCATGCTGAGG(BbvCI)GTGCCACTATAATATTAATGTGC |
| C03 | SIL1 ORF/ | CTAGATCTCTAGA(XbaI)ATGGTCCGGATTCTTCC |
| C04 | terminator | GCATGGCGCC(NarI)CCACGGCAGGGCAGTTGGCAC |
| C05 | JEM1 ORF/ | CTAGATCTCTAGA(XbaI)ATGATACTGATCTCGGG |
| C06 | terminator | CGATCGGTCCG(RsrII)AGGGAAATAAGGCAGATCAAAG |
| C07 | SCJ1 ORF/ | CACGCTTACTGCTTTTTTCTTCCCAAGATCGAAAATTTACTGAATTAA |
| | terminator | CAATGATTCCAAAATTATATATAC |
| C08 | | GCATCTCGAG(XhoI)GACTTTGAGACCTGTGATC |
| C09 | ADH1 | CGATCACCGATGTG(AleI)GTTGTTTCCGGGTGTACAATATGG |
| C10 | promoter | CCTATAGCAACAAAAGCTGTTAAAAATAAAAGCCTTAAAACGTTTCG |
| | | CATTGTATATGAGATAGTTGATTG |
| C11 | PGK1 | GCATGGGCCC(PspOMI)AGATTCCTGACTTCAACTCAAG |
| C12 | promoter | GATCTAGTCTAGA(XbaI)TGTTTTATATTTGTTGTAA |
| C13 | TDH1 | GCATGGCCGGCC(FseI)ACCATATGGAGGATAAGTTGG |
| C14 | promoter | ACCTAGTCTAGA(XbaI)TTTGTTTTGTGTGTAAATTTAG |
| C15 | TEF1 | CGGTAGTACCTGCAGG(SbfI)AAGCAACAGGCGCGTTGGAC |
| C16 | promoter | GGCAACAACAATAAAGATAGTATCAAATGTATATATAATTTTGGAAT |
| | | CATTTTGTAATTAAAACTTAGATTAGATTGC |
| C17 | HAC1 ORF | CTAGTCTCTAGA(XbaI)ATGGAAATGACTGATTTTGAAC |
| C18 | | CTAGTCTAGA(XbaI)TCATGAAGTGATGAAGAAATC |

Construction of pTPA01: 5′ and 3′ regions of the HO open reading frame were amplified by PCR from BY4741 (Brachmann et al., 1998, Yeast, 30; 14(2):115-32) genomic DNA using the primers A01-02 (5′) and A03-04 (3′). Fast Start High Fidelity PCR system (Roche) was used with the conditions as recommended, as defined in Example 3, above.

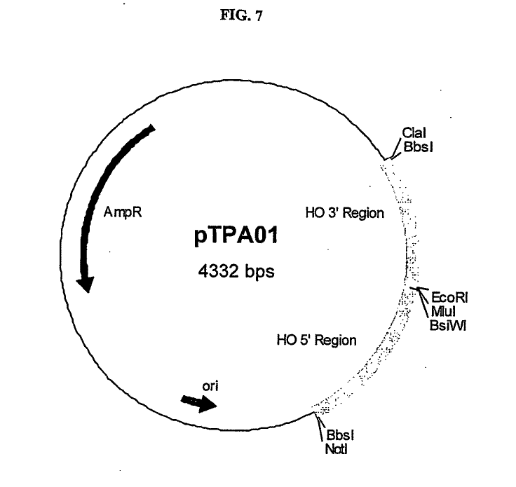

Fragments were gel extracted from a 1% (w/v) agarose TAE gel using the GeneClean III kit (Q-bio Gene). Purified DNA was digested with the appropriate enzymes, NotI and MluI for HO 5′ region, MluI and ClaI for HO 3′ region. pBST+ (WO99/00504) was digested with NotI and ClaI. Fragments were purified as above. A three-way ligation was performed using a Rapid Ligation Kit (Roche) as per manufacturers instructions. Ligations were transformed into the E. coli strain DH5α. Diagnostic restriction digests were performed on mini-prep DNA to confirm the ligation was successful. The plasmid map of TPA01 is shown in FIG. 7.

Polylinkers: To facilitate the cloning of the helper genes a polynucleotide linker was incorporated into pTPA01 (FIG. 7) and into YCplac33 (Gietz and Sugino, 1988, Gene, 74, 527-534).

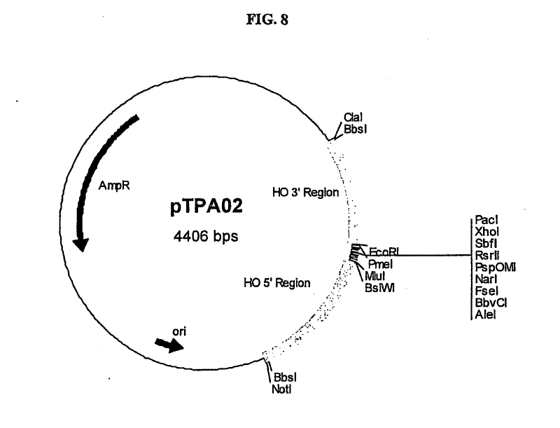

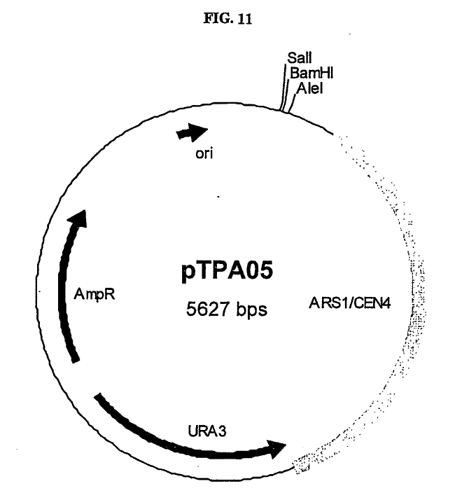

Complementary single stranded oligonucleotides were annealed as follows: 1 μL of a 100 μM solution of each oligo (A05-06 and A13-14) was added into a 50 μL, total volume containing 10× restriction buffer (Roche Buffer H for pTPA01 polylinker, Buffer B for YCplac33 polylinker). Samples were placed into a PCR machine and heated to 98° C. for 4 mins. Samples were then held for 1 min with the temperature dropping 1° C. every cycle down to 30° C. The annealed polylinkers were then digested by addition of the appropriate restriction enzyme (MluI, EcoRI for pTPA01 polylinker, BamHI, EcoRI for YCplac33 polylinker). Digested polylinkers were gel extracted as previously and ligated into the corresponding vector digests. Incorporation of polylinkers was confirmed by linearising plasmids with all restriction sites present in polylinkers. Vectors produced are shown as FIGS. 8 and 11 respectively.

Production of promoter/oven reading frame constructs: LHS1, SIL1, JEM1 and SCJ1 open reading frames (ORFs) plus approximately 300 bp of terminator sequence (3′ of ORF) and promoters were amplified by PCR, from the genomic DNA of an AH22 derivative, using Vent polymerase (NEB) (see Table 3 for primers used). Reactions were setup as per manufacturers instructions with an annealing temperature of 50° C. All fragments were gel extracted and resuspended in 5 μL of water. 1 μL was run on a gel to check fragment presence and quantity.

Promoters and ORFs for LHS1 and SCJ1 were joined according to the method of Shevchuk et al. (Nucleic Acids Res., 2004, 32(2), e19.). 100 ng of the ORF fragment and an equimolar amount of promoter fragment was used in the first PCR stage. 10 μL from this was used in the second PCR stage. Primers were added to a final concentration of 0.4 μM.

Second stage PCRs were run on a 1% (w/v) agarose TAE gel and bands extracted of the expected size (promoter+ORF+terminator). Extracted fragments were A-tailed using Fast Start High Fidelity polymerase (Roche) and cloned into the TOPO pCR2.1 vector (Invitrogen). Plasmid DNA was restriction digested to confirm the correct insert.

Promoters and ORFs for SIL1 and JEM1 were digested with restriction enzymes corresponding to sites incorporated into primers used for PCR (see Table 3). Promoter and ORF fragments were then joined by three way ligation with digested pTPA02.

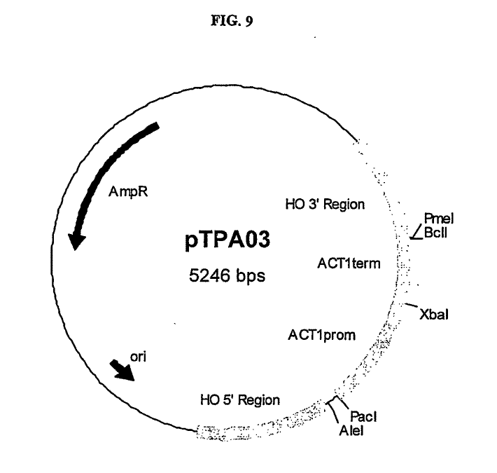

The ACT1 promoter and terminator were amplified by PCR from the genomic DNA of an AH22 derivative and gel extracted. Purified fragments were digested with restriction enzymes corresponding to sites incorporated into primers used for PCR and ligated in a three way ligation with PacI/PmeI digested pTPA02 to create pTPA03 (FIG. 9).

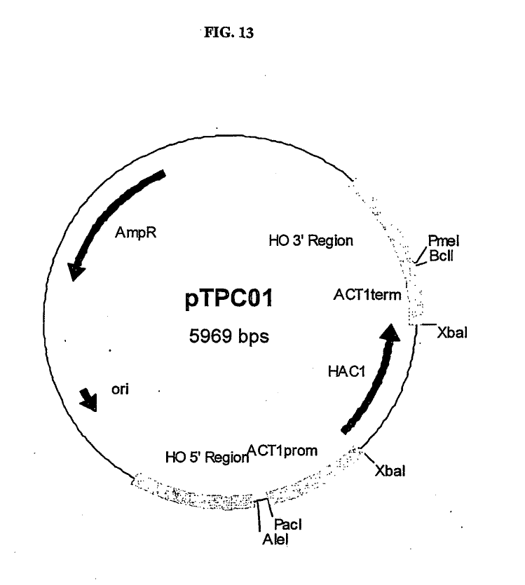

The HAC1 ORF was amplified by PCR from cDNA derived from RNA from an AH22 derivative treated with the reducing agent dithiothreitol (DTT). The spliced form of HAC1 (HAC1ⁱ) was identified as a 717 bp fragment and gel extracted. The extracted fragment was then digested with XbaI and ligated into pTPA03 digested with the same enzyme. Diagnostic restriction digests were used to confirm that the HAC1 ORF was present in the correct orientation relative to the ACT1 promoter and terminator sequences. The resultant plasmid pTPC01 is shown in FIG. 13.

All ORFs were sequenced and, with exception of LHS1, were shown to contain the same sequence as that published for the strain S288C. Repeat sequencing of multiple cloned PCR products for LHS1 confirmed that the AH22 derived clones contained a single base change from the S288C sequence. The base change at position 1215 (relative to the first base of the start codon) results in a change from A to C, which produces a Lys to Asn substitution at position 405.

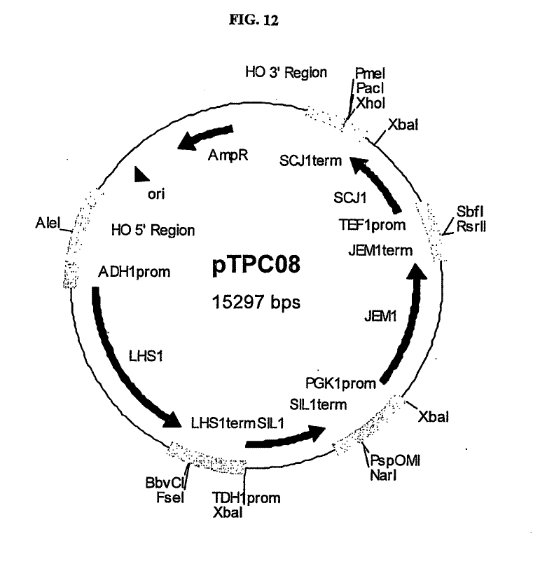

Assembly of overexpression constructs: Restriction digests (see Table 3) were performed to release promoter/ORF constructs from TOPO pCR2.1 vectors. Fragments were gel extracted and ligated into the pTPA02 vector, digested accordingly. In the first instance, constructs were produced containing each individual promoter/ORF and then containing all four. This required subsequent rounds of plasmid transformation, digestion and ligation. The vector containing all four promoter/ORFs is shown in FIG. 12.

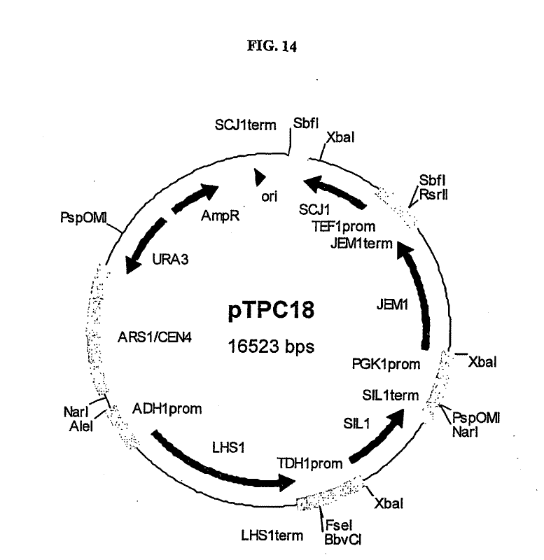

For insertion of the various promoter/ORF constructs (with the exception of HAC1) into the centromeric vector, pTPA05 (FIG. 11), an AleI/XhoI digest was performed on the various pTPA02 based vectors containing the required promoter/ORFs (e.g. pTPC08 (FIG. 12) for LHS1, SIL1, JEM1 and SCJ1), and an AleI/SalI digest on pTPA05 (FIG. 11). The various promoter/ORF fragments released were ligated into AleI/Sa/I digested pTPA05 to create a series of vectors including pTPC18 (FIG. 14) containing all four promoter/ORFs.

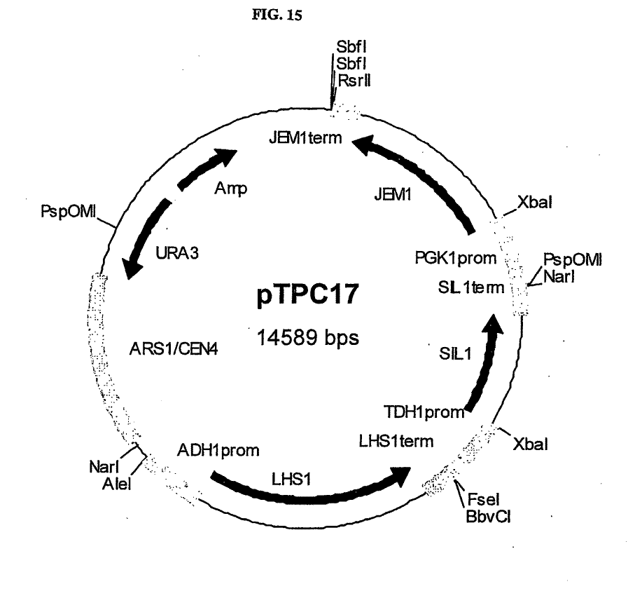

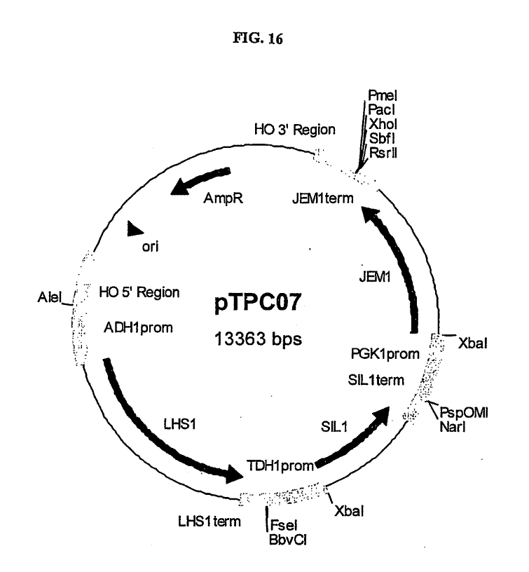

Plasmid pTPC17 (Example 4, FIG. 15) contained the LHS1, SIL1 and JEM1 ORFs expressed from YCplac33. pTPC17 was constructed by cloning an approximately 9.0-kb AleI-XhoI DNA fragment from pTPC07 (FIG. 16) that contained the expression cassette for the LHS1, SIL1 and JEM1 ORFs, into pTPA05 (FIG. 11) which had been digested with AleI and SalI. The expression cassette for the LHS1, SIL1 and JEM1 ORFs was assembled in pTPA05 in a similar method to that described for pTPC08 (FIG. 12), but using the promoter/ORF constructs from TOPO pCR2.1 vectors for LHS1, SIL1 and JEM1 expression.

For insertion of the HAC1 promoter/ORF (FIG. 13) into the centromeric vector pTPA05, an AleI/BclI digest was performed on pTPC01 (FIG. 13) and an AleI/BamHI digest was performed on pTPA05 (FIG. 11). The HAC1 AleI/BclI fragment released from pTPC01 was ligated into the AleI/BamHI digested pTPA05.

The various promoter/ORF constructs comprising the YCplac33 based plasmids pTPC11, pTPC12, pTPC13, pTPC14, pTPC15, pTPC17 and pTPC18 are shown in Table 4.

TABLE 4
Plasmid compositions

| Name | Helper genes overexpressed | Promoter used |
|---|---|---|
| YCplac33 | — | — |
| pTPC11 | HAC1ⁱ | ACT1 |
| pTPC12 | SIL1 | TDH1 |
| pTPC13 | LHS1 | ADH1 |
| pTPC14 | JEM1 | PGK1 |
| pTPC15 | SCJ1 | TEF1 |
| pTPC17 | LHS1, JEM1, SIL1 | As shown individually above |
| pTPC18 | LHS1, JEM1, SIL1, SCJ1 | As shown individually above |

Insertion of URA3 marker into pTPA02: The URA3 marker was amplified by PCR from the vector YCp50 as described above in Example 3. The fragment was gel extracted, digested with PacI/PmeI and ligated into each pTPA02 based vector containing the required promoter/ORFs (also PacI/PmeI digested). It is important the URA3 fragment be introduced last as it contains sites for restriction enzymes used elsewhere in construction of the plasmid.

Chromosomal integration: The helper gene constructs were integrated into the genome of a S. cerevisiae host cell by digestion of the vector pTPC08 (FIG. 12) with Nod and SacII and transformation of a ura3 derivative of AH22 [pAYE329] as described in Example 3, above.

Alternatively, the helper gene constructs may be introduced on a centromeric vector. For the YCplac33 based-vectors, 500 ng of plasmid DNA may be used to transform a S. cerevisiae host cell as above.

### EXAMPLE 5

Plasmids constructs were produced for the overexpression of the genes LHS1, JEM1, SCJ1 and SIL1 as described in Example 4, above.

The spliced form of the transcription factor HAC1 (referred to as HAC1ⁱ) was also overexpressed using the vector series produced. Due to the regulatory role of HAC1 within the unfolded protein response, HAC1s was overexpressed alone, not in conjunction with the other chaperone genes described here.

All genes were overexpressed from YCplac33 based vectors (Table 4) and transformed into the ura3 auxotrophic mutant of the ancestral S. cerevisiae strain (a histidine revertant of AH22) [pAYE329] defined in Example 4, above.

Overexpression was confirmed using real time PCR. Taqman hybridisation probes were designed to bind specifically to each gene under investigation plus ACT1, used here as an endogenous control. An additional probe was designed for the gene HAC1 to bind across the exon-exon junction—resulting in binding only to the spliced form. The proportion of HAC1ⁱ relative to total HAC1 can thus be determined.

TABLE 5
Taqman probe/primer sequences and binding co-ordinates

| | | |
|---|---|---|
| Gene Name | Feature | Sequence/Coordinates |

| | | |
|---|---|---|
| ACT1 | Forward primer (5′-3′) | CCCAGAAGCTTTGTTCCATCCTT |
| | Reverse primer (5′-3′) | ATGATGGAGTTGTAAGTAGTTTGGTCAA |
| | Probe (5′-3′) | CAGATTCCAAACCCAAAACA |
| | Coordinates* | 795-814 |
| LHS1 | Forward primer (5′-3′) | ACACTACTCAGCCCGTTACAATAGA |
| | Reverse primer (5′-3′) | GTAAACTTTGCACCACCTAGATGTG |
| | Probe (5′-3′) | ATTTGAAGGATATGGGTATAATC |
| | Coordinates* | 789-811 |
| SIL1 | Forward primer (5′-3′) | GACATGTACGAAAATGACGATACAAATCT |
| | Reverse primer (5′-3′) | TCGTTTGCCCACTCTTGCA |
| | Probe (5′-3′) | TTTGACGACCAATTCTC |
| | Coordinates* | 940-956 |
| SCJ1 | Forward primer (5′-3′) | GGCGCAGGTGGATTCCA |
| | Reverse primer (5′-3′) | CGCCAGGACCTCCATGAC |
| | Probe (5′-3′) | CATATTCGAACGGATGTTTC |
| | Coordinates* | 342-361 |
| JEM1 | Forward primer (5′-3′) | CCTCTCCACGCACATCGA |
| | Reverse primer (5′-3′) | TGCTTGTCGAGGATTGTTTCGTAAT |
| | Probe (5′-3′) | TCGTTAGCTGCTGCTATCA |
| | Coordinates* | 592-610 |
| HAC1 | Forward primer (5′-3′) | GAAGACGCGTTGACTTGCA |
| | Reverse primer (5′-3′) | GAAATCCCTGTACTCGTCAAGAGAA |
| | Probe (5′-3′) | CCACGACGCTTTTGTTGC |
| | Coordinates* | 288-305 |
| HAC1ⁱ | Forward primer (5′-3′) | ACAATTCAATTGATCTTGACAATTGG |
| | Reverse primer (5′-3′) | TCAATTCAAATGAATCAAACCTGAC |
| | Probe (5′-3′) | CGTAATCCAGAAGCGCA |
| | Coordinates* | 652-668 |

*means probe binding coordinates, relative to start codon

The relative standard curve method of transcript quantification was used as described by Applied Biosystems in the ‘ABI PRISM 770 Sequence Detection System: User Bulletin #2’ document. This can be downloaded from the Applied Biosystems' website (www.appliedbiosystems.com). Equivalent technical disclosure of a suitable quantitative RT-PCR method can be found in Bustin, 2000, Journal of Molecular Endocrinology, 25, 169-193. This method allows quantification of the gene of interest relative to an endogenous control gene that is known to exhibit constant expression across experimental conditions.

All real time PCR was carried out on cDNA derived from RNA extracted from log phase (OD₆₀₀=2) BMMD yeast cultures. Overexpression was assessed by comparison of strains with a control yeast strain transformed with the base vector YCplac33 and are expressed as fold changes.

TABLE 6
Summary of overexpression levels achieved

| | Overexpression in | |
|---|---|---|
| | single gene construct | Overexpression in multiple gene |
| | (Fold change vs. | construct pTPC18 |
| Gene | YCplac33 control) | (Fold change vs. YCplac33 control) |
| HAC1ⁱ | 3.51 | — |
| LHS1 | 22.63 | 23.52 |
| JEM1 | 10.16 | 11.48 |
| SIL1 | 2.03 | 2.36 |
| SCJ1 | 15.81 | 16.71 |

As shown below in Table 6, overexpression levels vary between the different constructs. Levels achieved range from 2.03 fold for SIL1 to 22.63 fold for LHS1.

The effect of overexpression of HAC1ⁱ, LHS1, JEM1, SIL1 and SCJ1 on the induction of the stress-related unfolded protein response (UPR) in a host cell was investigated by measuring the levels of HAC1ⁱ and total HAC1 transcript levels in AH22 (ura3) [pAYE329] host cells transformed with Ycplac33 (as a negative control), pTPC11, pTPC12, pTPC13, pTPC14, pTPC15 or pTPC18. Total HAC1 transcript levels are the sum of HAC1ⁱ transcript levels and unspliced HAC1 transcript levels. A reduced proportion of the level of HAC1ⁱ transcript levels compared to total HAC1 transcript levels is indicative of reduced stress and reduced UPR signalling.

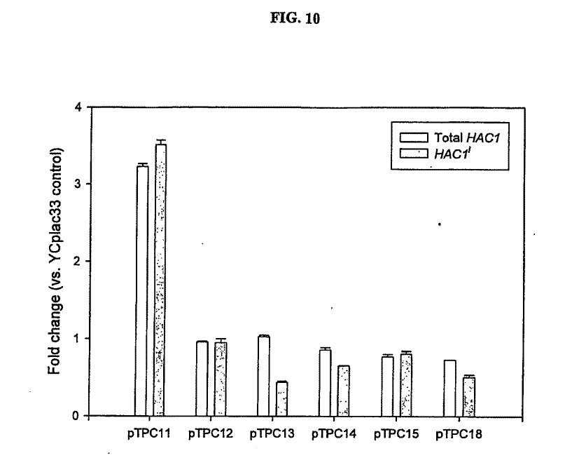

FIG. 10 shows that individual over-expression of LHS1 (pTPC13) or JEW (pTPC14) or simultaneous over-expression of all of LHS1, JEM1, SIL1 and SCJ1 (pTPC18) resulted a reduced proportion of the level of HAC1ⁱ transcript levels (compared to total HAC1 transcript levels) compared to the control. This indicates that over-expression of the above-identified helper proteins can help to reduce stress in cultured cells and avoid the unnecessary induction of the UPR.

### EXAMPLE 6

The levels of recombinant protein production achieved by the transformed strains described in Examples 4 and 5 (see Table 4), above, were analysed. In this case, the recombinant protein was recombinant human albumin (“rHA”) expressed from the plasmid pAYE329, described in Sleep et al.; 1990, Gene, 101, 89-96.

All analysis was performed on cultures grown for 5 days at 30° C., 200 rpm.

Culture supernatants were run immediately on gels to prevent any rHA proteolysis/degradation that could otherwise occur during freezing and overnight storage at −20° C. Each of the three bands (main rHA band plus two degradation products) were quantified by densitometry. This gives an indication of rHA production levels and the level of proteolysis occurring in each strain. The mutagenised strain identified in Example 1 was also included as a positive control.

Results of the analysis are shown in FIG. 17. It is apparent from a comparison of the results for the ancestral strain expressing recombinant albumin from pAYE329/YCplac33 (“YCplac33”) and the mutagenised strain identified in. Example 1 as possessing increased recombinant protein production (“+ve control”) that the mutagenised strain is not only capable of producing increased levels of rHA, but additionally displays reduced levels of rHA degradation compared to the ancestral strain. Moreover, FIG. 17 is particularly clear in demonstrating that strain transformed with pTPC17 (i.e. the ancestral strain transformed to over-express LHS1, JEM1 and SIL1) also displays reduced levels of rHA degradation compared to the untransformed ancestral strain.

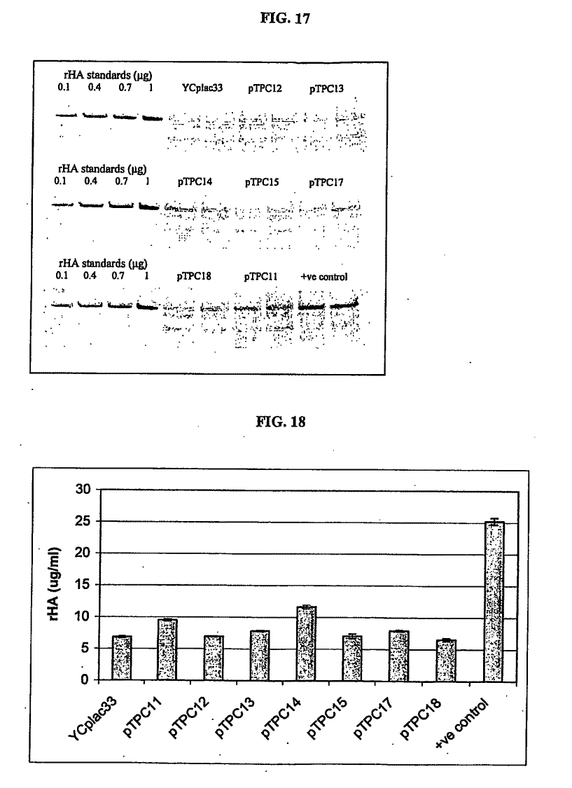

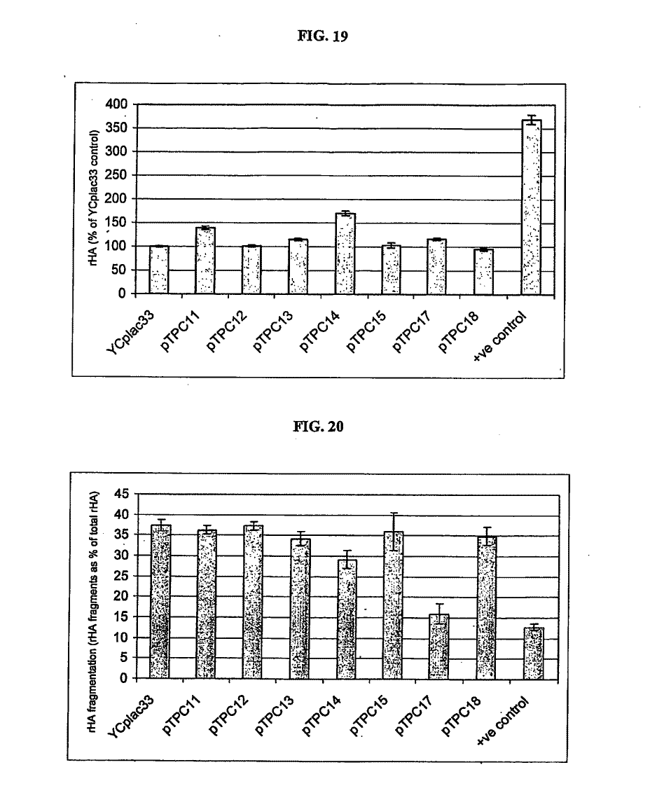

Further characterisation of the effect of the defined transformations is possible in view of the analysis of the SDS-PAGE gel by densitometry, the results of which are present in Table 7, below, and FIGS. 18 and 19.

TABLE 7
Comparison of rHA levels, as percentage of YCplac33 control production
levels. In the third column, the rHA production levels have been
normalised (based on culture optical density readings) to account for
different growth rates observed between transformants.

| | |
|---|---|
| | rHA production, |
| Overexpression | as % of YCplac33 control |

| plasmid | Not normalised by OD | Normalised by OD |
|---|---|---|
| pTPC11 | 164.26 | 139.2 |
| pTPC12 | 102.51 | 101.7 |
| pTPC13 | 122.42 | 115.1 |
| pTPC14 | 177.85 | 170.4 |
| pTPC15 | 86.37 | 103.4 |
| pTPC17 | 132.85 | 116.3 |
| pTPC18 | 102.65 | 96.0 |
| +ve control | 383.44 | 369.0 |

Table 7, above, and FIGS. 18 and 19, show that the individual overexpression of HAC1, LHS7, JEM1, SIL1 and SCJ1 results in an increase in rHA production, on a per cell basis (i.e. when results are normalised by culture OD). However, the negative growth effect of SCJ1 overexpression resulted in an overall reduction of rHA production on a per culture basis (i.e. when results are not normalised by culture OD).

The overexpression of JEM1 alone had the largest measured effect on rHA production.

However, as will be apparent from FIG. 17, the strains that individually expressed HAC1, LHS1, JEM1, SIL1 and SCJ1 still demonstrated relatively high levels of rHA degradation, comparable to the ancestral strain and higher than the to mutagenised strain identified in Example 1. By contrast, cells that simultaneously over-express LHS1, JEM1 and SIL1 demonstrate increased rHA productivity and a concomitant reduction in rHA degradation, comparable with the mutagenised strain identified in Example 1. This is further demonstrated in FIG. 20. In fact, FIG. 20 shows that several of the strains tested show lower levels of degradation compared to the ancestral strain, but this reduction is particularly pronounced in strain transformed with pTPC17.

### EXAMPLE 7

This example describes the increased secretion of a recombinant transferrin mutant by over-expression of LHS1, JEM1 and SIL1 from the centromeric vector pTPC17 in a Saccharomyces cerevisiae strain containing a 2-micron plasmid encoding the PDI1 gene.

A S. cerevisiae strain, the “control strain” as used in WO 2005/061718 and WO 2005/061719 was used to generate a ura3 mutant derivative, referred to herein as “control strain (ura3)” by random mutagenesis and selection on 5-fluoro-orotic acid plates (Boeke et al., 1984, op. cit.).

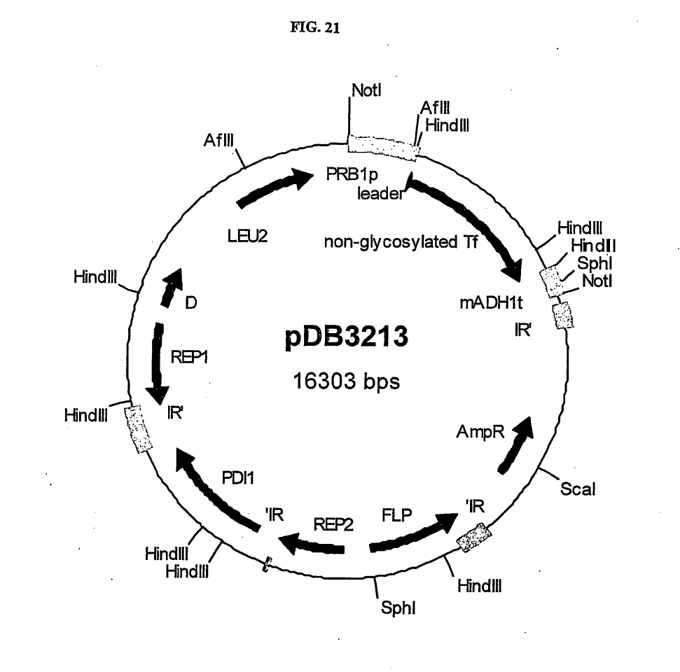

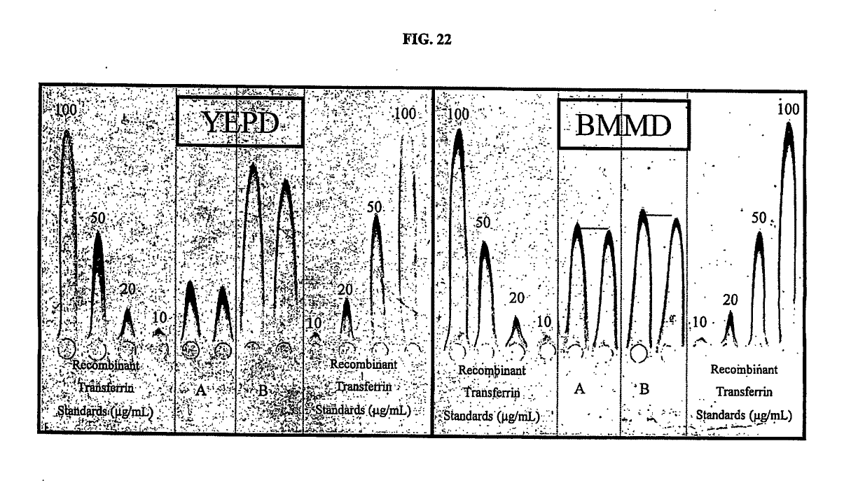

The S. cerevisiae control strain was transformed to leucine prototrophy with pDB3213 (FIG. 21) and the control strain (ura3) was co-transformed to both leucine and uracil prototrophy with plasmids pTPC17 (FIG. 15) and pDB3213. Transformation was by a modified lithium acetate method (Sigma yeast transformation kit, YEAST-1, protocol 2 (Elble, R, 1992, Biotechniques, 13, 18-20; Ito et al., 1983, op. cit.). Transformants were selected on BMMD-agar plates, and subsequently patched out on BMMD-agar plates.

The construction of pTPC17 is described in Example 4.

Plasmid pDB3213 is similar to pDB2929 (WO 2005/061718, Example 1 and FIG. 12), and contains a NotI expression cassette for a non-glycosylated transferrin cloned into pDB2690 (WO 2005/061718, Example 1 and FIG. 6). The NotI expression cassette of pDB3213 contains an alternative codon for Leucine-505 in mature transferrin that is the CTG codon (11% codon usage in S. cerevisiae) compared to the CTG codon (6% codon usage in S. cerevisiae) present in pDB2929, a KEX2-independent leader sequence (derived from the HSA-pre leader sequence) and mutations within the N-linked glycosylation sites (-N-X-S/T-) that prevent glycosylation of residues N413 and N611.

Transformants of each strain were inoculated into 10 mL BMMD and 10 mL YEPD in 50 mL shake flasks and incubated in an orbital shaker at 30° C., 200 rpm for 4-days. Culture supernatants were harvested and the recombinant transferrin titres compared by rocket immunoelectrophoresis (FIG. 22). The results indicated that the recombinant transferrin titres in supernatants of both the YEPD and BMMD shake flask cultures were higher when pTPC17 was present. Furthermore, in high cell density fed batch fermentation the recombinant transferrin titres from control strain (ura3) [pTPC17 pDB3213] was 1.7 g/L compared to only 0.9 g/L for control strain [pDB3213]. Therefore, over-expression of LHS1, JEM1 and SIL1 from the centromeric plasmid pTPC17 had approximately doubled the quantity of the recombinant transferrin product secreted from the S. cerevisiae strain during fermentation.

It is to be noted that pDB3213 encodes an additional copy of PDI1, and these results suggest that over-expression of PDI1 (and variants thereof) in conjunction with one, two or all three of LHS1, JEM1, and SIL1 (e.g. LHS1 alone; JEM1 alone; SIL1 alone; LHS1 and JEM1; LHS1, and SIL1; JEM1, and SIL1; or LHS1, JEM1, and SIL1) provide unexpected benefits to the production of a desired protein product.

### EXAMPLE 8

This example shows increased secretion of recombinant albumin (“rHA”) by over-expression of LHS1, JEM1 and SIL1 from the centromeric vector pTPC17 in a Saccharomyces cerevisiae strain.

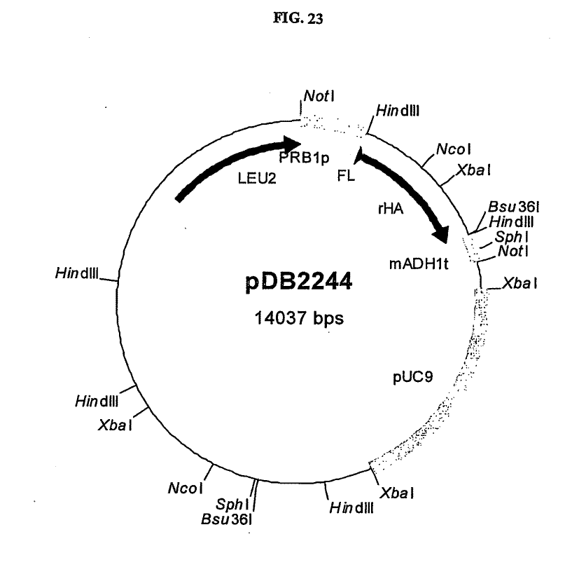

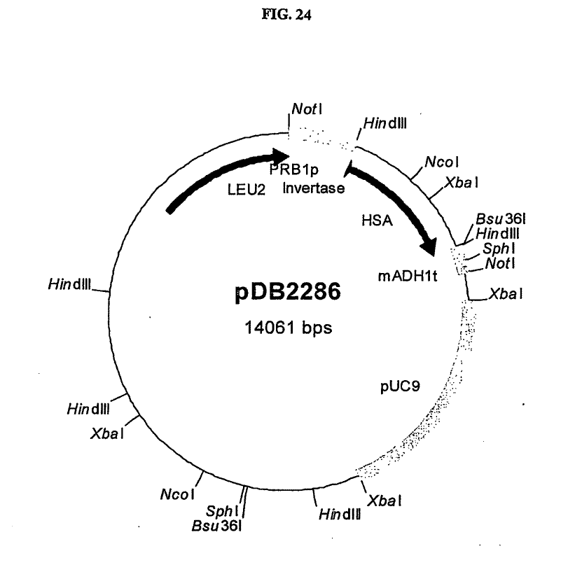

Construction of plasmid pDB2243 containing the NotI rHA expression cassette, incorporating the HSA/MFα-1 fusion leader sequence, as taught in WO 90/01063, is described in WO 00/44772 (see WO 00/44772, FIG. 6). The rHA expression disintegration vector pDB2244 (FIG. 23) was created by ligating the NotI expression cassette from pDB2243 into NotI cut pSAC35 (Sleep et al, 1991, Bio/Technology 9, 183-187 and EP 431 880) to generate the plasmid pDB2244 in which the direction of rHA transcription is in the same orientation as that of the LEU2 gene as described in WO 00/44772.

Construction of plasmid pDB2283 containing a NotI rHA expression cassette, incorporating the invertase leader sequence, was accomplished by replacing the 1.21-kb BfrI-XbaI fragment in pDB2243, comprising the HSA/MFα-1 fusion leader sequence and part of the human albumin cDNA, with a 1.07-kb blunt end-XbaI fragment from mp 19.7 (EP-A-248 637) and a synthetic double stranded oligonucleotide linker of the following structure—

| | |
|---|---|
| 1 | gagtccaatt agcttcatcg ccaataaaaa aacaagctaa acctaattct |
| | ctcaggttaa tcgaagtagc ggttattttt ttgttcgatt tggattaaga |
| | HindIII |
| | -+---- |
| 51 | aacaagcaaa gatgaagtgg gtaagcttaa cctaattcta acaagcaaag |
| | ttgttcgttt ctacttcacc cattcgaatt ggattaagat tgttcgtttc |
| 101 | atgcttttgc aagccttcct tttccttttg gctggttttg cagccaaaat |
| | tacgaaaacg ttcggaagga aaaggaaaac cgaccaaaac gtcggtttta |
| | >>......................Invertase......................> |
| | m l l q a f l f l l a g f a a k |
| 151 | atctgca |
| | tagacgt |
| | >....>> Invertase |
| | i s a |

which was formed by annealing two complementary single stranded oligonucleotides with the sequences

5′TTAAGAGTCCAATTAGCTTCATCGCCAATAAAAAAACAAGCTAAACCT
AATTCTAACAAGCAAAGATGAAGTGGGTAAGCTTAACCTAATTCTAACAA
GCAAAGATGCTTTTGCAAGCCTTCCTTTTCCTTTTGGCTGGTTTTGCAGC
CAAAATATCTGCA3′;
and
5′TGCAGATATTTTGGCTGCAAAACCAGCCAAAAGGAAAAGGAAGGCTTG
CAAAAGCATCTTTGCTTGTTAGAATTAGGTTAAGCTTACCCACTTCATCT
TTGCTTGTTAGAATTAGGTTTAGCTTGTTTTTTTATTGGCGATGAAGCTA
ATTGGACTC3′.

Plasmid mp 19.7 (EP-A-248 637) was digested to completion with XhoI, phenol/chloroform extracted and ethanol precipitated. The recovered DNA was then blunt ended with the Klenow fragment of E. coli DNA polymerase I to remove the XhoI overhang, phenol/chloroform extracted, and ethanol precipitated. The recovered DNA was digested to completion with XbaI. The digestion products were resolved by agarose gel electrophoresis and the 1.07-kb blunt end-XbaI mp 19.7 fragment recovered using the GeneClean III kit (Q-bio Gene).

The rHA expression disintegration vector pDB2286 (FIG. 24) was created by ligating the Nod expression cassette from pDB2283 into NotI cut pSAC35 (Sleep et al, 1991, Bio/Technology 9, 183-187 and EP 431 880).

Construction of plasmid pDB2284 containing a Nod rHA expression cassette, incorporating the MFα-1 leader sequence, was accomplished by replacing the 1.21-kb BrfI-XbaI fragment in pDB2243, comprising the HSA/MFα-1 fusion leader sequence and part of the human albumin cDNA, with a 1.07-kb blunt end-XbaI fragment from mp 19.7 (EP-A-248 637) and a synthetic double stranded phosphorylated oligonucleotide linker of the structure—

| | |
|---|---|
| 1 | ttaagagtcc aattagcttc atcgccaata aaaaaacaaa ctaaacctaa |
| | ctcagg ttaatcgaag tagcggttat ttttttgttt gatttggatt |
| | PstI |
| | ------+ |
| 51 | ttctaacaag caaagatgag atttccttca atttttactg cagttttatt |
| | aagattgttc gtttctactc taaaggaagt taaaaatgac gtcaaaataa |
| | >>.............MFalpha..............> |
| | m r f p s i f t a v l |
| 101 | cgcagcatcc tccgcattag ctgctccagt caacactaca acagaagatg |
| | gcgtcgtagg aggcgtaatc gacgaggtca gttgtgatgt tgtcttctac |
| | > MFalpha > |
| | f a a s s a l a a p v n t t t e d |
| 151 | aaacggcaca aattccggct gaagctgtca tcggttactc agatttagaa |
| | tttgccgtgt ttaaggccga cttcgacagt agccaatgag tctaaatctt |
| | > MFalpha > |
| | e t a q i p a e a v i g y s d l e |
| 201 | ggggatttcg atgttgctgt tttgccattt tccaacagca caaataacgg |
| | cccctaaagc tacaacgaca aaacggtaaa aggttgtcgt gtttattgcc |
| | > MFalpha > |
| | g d f d v a v l p f s n s t n n |
| 251 | gttattgttt ataaatacta ctattgccag cattgctgct aaagaagaag |
| | caataacaaa tatttatgat gataacggtc gtaacgacga tttcttcttc |
| | > MFalpha > |
| | g l l f i n t t i a s i a a k e e |
| | HindIII |
| | -+---- |
| 301 | gggtaagctt ggataaaaga |
| | cccattcgaa cctattttct |
| | >......MFalpha.....>> |
| | g v s l d k r |

formed by annealing complementary six single stranded oligonucleotides with the sequences

5′TTAAGAGTCCAATTAGCTTCATCGCCAATAAAAAAACAAACTAAACCT
AATTCTAACAAGCAAAGATGAGATTTCCTTCAATTTTTACTGCAGTTTTA
3′;
5′TTCGCAGCATCCTCCGCATTAGCTGCTCCAGTCAACACTACAACAGAA
GATGAAACGGCACAAATTCCGGCTGAAGCTGTCATCGGTTACTCAGATTT
AGAAGGGGATTT3′;
5′CGATGTTGCTGTTTTGCCATTTTCCAACAGCACAAATAACGGGTTATT
GTTTATAAATACTACTATTGCCAGCATTGCTGCTAAAGAAGAAGGGGTAA
GCTTGGATAAAAGA3′;
5′TCTTTTATCCAAGCTTACCCCTTCTTCTTTAGCAGCAATGCTGGCAAT
AGTAGTATTTATAAACAATAACCCGTTATTTGTGCTGTTGGAAAATGGCA
AAAC3′;
5′AGCAACATCGAAATCCCCTTCTAAATCTGAGTAACCGATGACAGCTTC
AGCCGGAATTTGTGCCGTTTCATCTTCTGTTGTAGTGTTGACTGGAGCAG
CTAATGCGGAGG3′;
and
5′ATGCTGCGAATAAAACTGCAGTAAAAATTGAAGGAAATCTCATCTTTG
CTTGTTAGAATTAGGTTTAGTTTGTTTTTTTATTGGCGATGAAGCTAATT
GGACTC3′.

Plasmid mp 19.7 (EP-A-248 637) was digested to completion with XhoI, phenol/chloroform extracted and ethanol precipitated. The recovered DNA was then blunt ended with the Klenow fragment of E. coli DNA polymerase I to remove the XhoI overhang, phenol/chloroform extracted, and ethanol precipitated. The recovered DNA was digested to completion with XbaI. The digestion products were resolved by agarose gel electrophoresis and the 1.07-kb blunt end-XbaI mp 19.7 fragment recovered using the GeneClean III kit (Q-bio Gene).

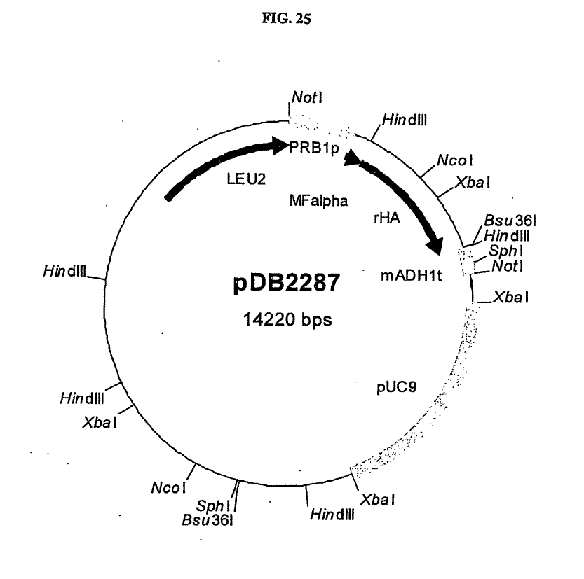

The rHA expression disintegration vector pDB2287 (FIG. 25) was created by ligating the NotI expression cassette from pDB2284 into NotI cut pSAC35 (Sleep et al, 1991, Bio/Technology 9, 183-187 and EP 431 880).

The ura3 auxotrophic mutant of the AH22 histidine revertant described in Example 4 was co-transformed to both leucine and uracil prototrophy with plasmids pDB2244 and YCplac33, or pDB2244 and pTPC17, or pDB2286 and YCplac33, or pDB2286 and pTPC17, or pDB2287 and YCplac33, or pDB2287 and pTPC17. Transformation was by a modified lithium acetate method (Sigma yeast transformation kit, YEAST-1, protocol 2 (Elble, 1992, op. cit.; Ito et al, 1983, op. cit.). Transformants were selected on BMMD-agar plates, and subsequently patched out on BMMD-agar plates.

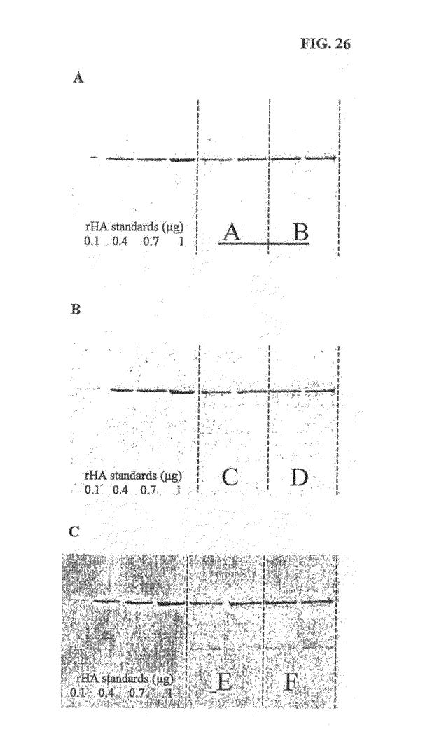

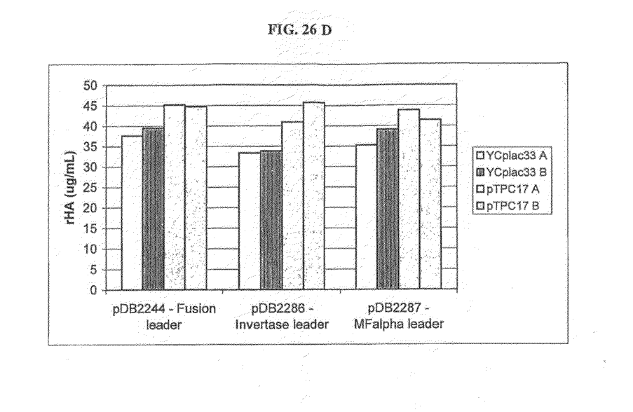

Two transformants for each strain were inoculated into 10 mL BMMD in 50 mL shake flasks and incubated in an orbital shaker at 30° C., 200 rpm for 4-days. Culture supernatants were harvested and the recombinant human albumin (rHA) titres compared by SDS-PAGE (FIG. 26 A-C) and densitometric analysis (FIG. 26 D). The results are summarised in Table 8, below.

TABLE 8
Increased rHA secretion by overexpression of SIL1, LHS1 and JEM1
(pTPC17) from three distinct leader sequences.

| | Average percentage increase |
|---|---|
| | in rHA secretion by pTPC17 |
| Expression plasmid | versus YCplac33 transformation |
| pDB2244 | 29.1 |
| pDB2286 | 16.7 |
| pDB2287 | 14.5 |

The results indicated that the rHA titres were increased by transformation with pTPC017 relative to the control plasmid YCplac33. Increases in rHA titres varied between the different expression constructs in the range of 14.5-29.1% demonstrating the beneficial effect of LHS1, JEM1 and SIL1 on rHA secretion was not restricted to a specific secretory leader sequence. Thus, for example, it is clear that the beneficial effect of LHS1, JEM1 and SIL1 on rHA secretion was not restricted by features of the leader sequence at the amino acid or DNA sequence level, or by configuration (pre or pre-pro) or whether or not the secretory leader sequence contained N-linked glycosylation sites.

### EXAMPLE 9

This example describes the increased secretion of recombinant granulocyte macrophage colony stimulating factor (GM-CSF) from a 2-micron based plasmid by over-expression of LHS1, JEM1 and SIL1 from the centromeric vector pTPC17.

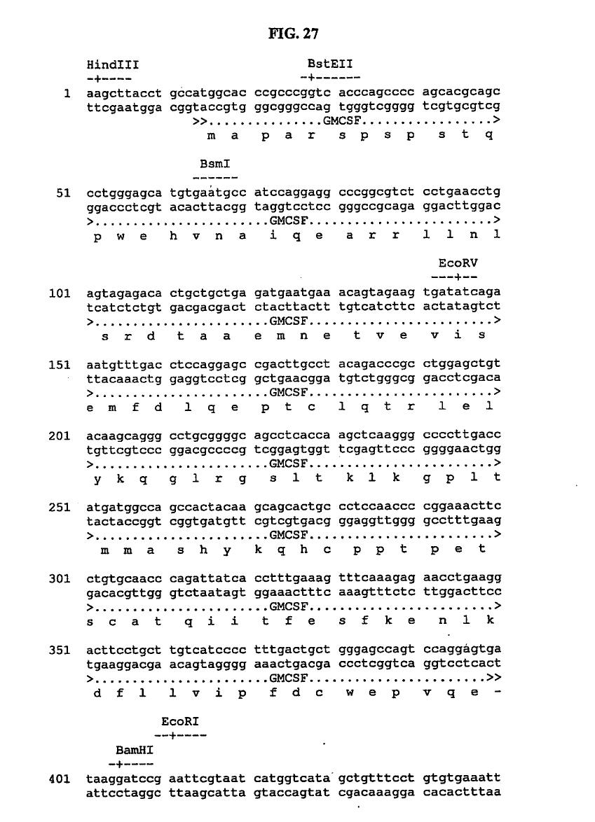

A cDNA for human GM-CSF was obtained from plasmid pBBG12 (R&D Systems Europe Ltd.) cloned between the HindIII and EcoRI sites of the pUC18 polylinker. The DNA sequence of the human GM-CSF cDNA (FIG. 27) incorporated an N-terminal Met codon.

Oligonucleotides SINK1 and SINK 2 were synthesised to construct a linker which would reconstruct the HSA/MFα-1 fusion leader as taught in WO 90/01063, coupled to GM-CSF up to the BstEII site.

SINK1: 5′GTACCAAGCTTTATTTCCCTTCTTTTTCTCTTTAGCTCGGC
TTATTCCAGGAGCTTGGATAAAAGAGCACCCGCCCG3′
SINK2: 5′GTGACCGGGCGGGTGCTCTTTTATCCAAGCTCCTGGAATAA
GCCGAGCTAAAGAGAAAAAGAAGGGAAATAAAGCTTG3′

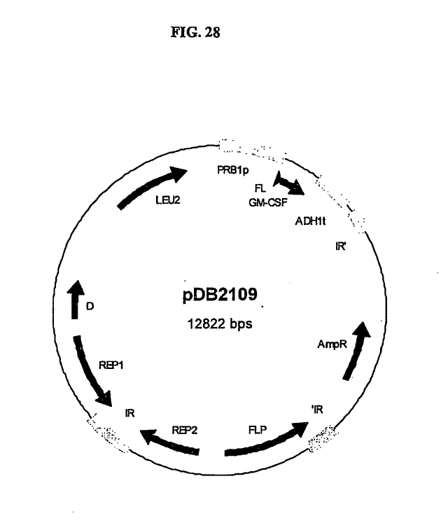

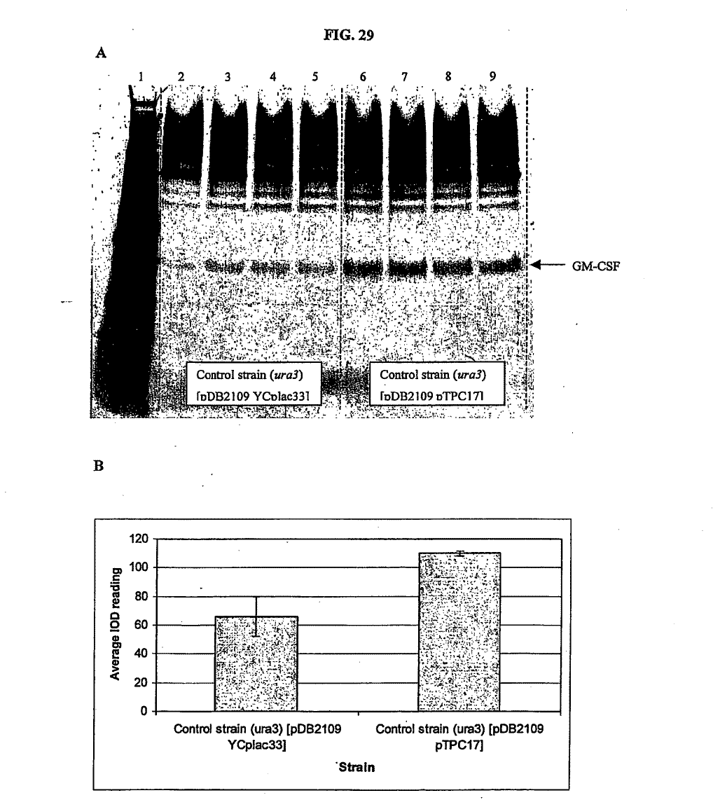

A 380 bp BstEII/BamHI GMCSF fragment was isolated from pBBG12 and ligated into pUC19 Asp718/BstEII along with the Asp718/BstEII SINK1/2 linker above, to create pDB2095. Accordingly, the GM-CSF cDNA, linked to the HSA/MFα-1 fusion secretion leader, was available on a HindIII fragment suitable for subcloning into pAYE441 (as described in WO 2004/009819, Example 1 and FIG. 5) to create pDB2102 in which the GM-CSF cDNA was now present on a NotI expression cassette, comprising the PRB1 promoter, the HSA/MFα-1 fusion secretion leader and the ADH1 terminator. The GM-CSF NotI expression cassette was isolated and subcloned into pSAC35 (Sleep et al, 1991; Biotechnology (NY), 9, 13 and EP 431 880) linearised with NotI to create plasmid pDB2109 (FIG. 28).

The S. cerevisiae Control Strain (ura3), as described above in Example 7, was co-transformed to both leucine and uracil prototrophy with plasmids pDB2109 (FIG. 28) and either YCplac33 or pTPC17 (FIG. 15). Transformation was by a modified lithium acetate method (Sigma yeast transformation kit, YEAST-1, protocol 2 (Elble, 1992, op. cit.; Ito et al., 1983, op. cit.). Transformants were selected on BMMD-agar plates, and subsequently patched out on BMMD-agar plates.

Transformants of each strain were inoculated into 10 mL BMMD in 50 mL shake flasks and incubated in an orbital shaker at 30° C., 200 rpm for 4-days. Culture supernatants were harvested and the recombinant GM-CSF titres compared by SDS-PAGE and densitometric analysis (FIGS. 29 A and B). The results of the densitometric analysis are also provided in Table 9, below.

TABLE 9
Increased GM-CSF production as determined by SDS-PAGE and
densitometric analysis

| | |
|---|---|
| Control strain | Control strain |
| (ura3) [pDB2109 YCplac33] | (ura3) [pDB2109 pTPC17] |

| | Integrated optical | | Integrated optical |
|---|---|---|---|
| Gel lane | density | Gel lane | density |
| 2 | 45.20 | 6 | 108.36 |
| 3 | 72.14 | 7 | 108.41 |
| 4 | 71.54 | 8 | 111.73 |
| 5 | 74.21 | 9 | 111.30 |
| Average | 65.77 | Average | 109.95 |

The results indicated that the recombinant GM-CSF titres in supernatants of BMMD shake flask cultures were greater than 50% higher when pTPC17 was present.

1-31. (canceled)

32. A host cell suitable for enhanced production of a protein product of choice characterised in that the host cell is genetically modified to cause over-expression of two or more helper proteins selected from a DnaJ-like protein (such as JEM1), an Hsp70 family protein (such as LHS1) and SIL1, wherein at least one of the over-expressed two or more helper proteins is selected from JEM1, LHS1 and SIL1, and wherein the DnaJ-like protein is not SCJ1.

33. The host cell of claim 32 wherein the host cell is genetically modified to cause over-expression of
(a) a DnaJ-like protein and an Hsp70 family protein; or
(b) a DnaJ-like protein and SIL1; or
(c) an Hsp70 family protein and SIL1.

34. A host cell suitable for enhanced production of a protein product of choice characterised in that the host cell is genetically modified to cause over-expression of three or more helper proteins, wherein the three or more helper proteins comprise a DnaJ-like protein, an Hsp70 family protein and SIL1, and wherein the DnaJ-like protein is not SCJ1.

35. The host cell of claim 32 wherein the Hsp70 family protein is a protein that localises to the lumen of the ER.

36. The host cell of claim 2 wherein the Hsp70 family protein is not a prokaryotic Hsp70 family protein.

37. The host cell of claim 32 according to any one of the preceding claims wherein the Hsp70 family protein is LHS1, KAR2, SSA1, SSA2, SSA3, SSA4, SSE1, SSE2, SSB1, SSB2 or ECM10.

38. The host cell of claim 32 wherein the DnaJ-like protein is a protein that localises to the ER membrane.

39. The host cell of claim 32 wherein the DnaJ-like protein is selected from JEM1, MDJ2, SEC63, YDJ1, XDJ1, APJ1, SIS1, DJP1, ZUO1, SWA2, JJJ1, JJJ2, JJJ3, CAJ1, CWC23, PAM18, JAC1, JID1, SCJ1, HLJ1 and ERJ5.

40. The host cell of claim 32 wherein the host cell is further genetically modified to cause over-expression of at least one, two, three, four, five, six or seven proteins involved in the formation of disulphide bonds in other proteins selected from the group consisting of ERO1, ERV2, EUG1, MPD1, MPD2, EPS1 and PDI1.

41. The host cell that is suitable for enhanced production of a protein product of choice characterised in that the host cell comprises a first gene encoding a first helper protein selected from JEM1, LHS1 or SIL1 or a variant thereof, and a second gene encoding a desired protein product of choice, wherein the host cell is genetically modified to cause over-expression of the first helper protein, and
(a) wherein the first and second genes are not both present within the host cell on the same 2 μm-family plasmid; and
(b) wherein the host cell is not genetically modified to cause over-expression of a further helper protein that is different from the first helper protein and is selected from the group consisting of AHA1, CCT2, CCT3, CCT4, CCT5, CCT6, CCT7, CCT8, CNS1, CPR3, CPR6, ERO1, EUG1, FMO1, HCH1, HSP10, HSP12, HSP104, HSP26, HSP30, HSP42, HSP60, HSP78, HSP82, JEM1, MDJ1, MDJ2, MPD1, MPD2, PDI1, PFD1, ABC1, APJ1, ATP11, ATP12, BTT1, CDC37, CPR7, HSC82, KAR2, LHS1, MGE1, MRS11, NOB1, ECM10, SSA1, SSA2, SSA3, SSA4, SSC1, SSE2, SIL1, SLS1, ORM1, ORM2, PER1, PTC2, PSE1, UBI4 and HAC1 or a truncated intronless HAC1.

42. The host cell of claim 41, wherein the first helper protein is JEM1, LHS1 or SIL1.

43. The host cell of claim 42 wherein the first helper protein is the only helper protein that is over-expressed by the host cell.

44. The host cell of claim 32 wherein the protein product of choice is a heterologous protein and/or comprises a leader sequence effective to cause secretion.

45. The host cell of claim 32 wherein the protein product of choice is a eukaryotic protein, or a fragment or variant thereof.

46. The host cell of claim 32 wherein the protein product of choice comprises albumin, a monoclonal antibody, an etoposide, a serum protein, antistasin, a tick anticoagulant peptide, transferrin, lactoferrin, endostatin, angiostatin, collagens, immunoglobulins, or immunoglobulin-based molecules or fragment of either, a Kunitz domain protein, interferons, interleukins, leptin, CNTF and fragments thereof, IL1-receptor antagonist, erythropoietin (EPO) and EPO mimics, thrombopoietin (TPO) and TPO mimics, prosaptide, cyanovirin-N, 5-helix, T20 peptide, T1249 peptide, HIV gp41, HIV gp120, urokinase, prourokinase, tPA, hirudin, platelet derived growth factor, parathyroid hormone, proinsulin, insulin, glucagon, glucagon-like peptides, insulin-like growth factor, calcitonin, growth hormone, transforming growth factor beta, tumour necrosis factor, G-CSF, GM-CSF, M-CSF, FGF, coagulation factors in both pre and active forms, including but not limited to plasminogen, fibrinogen, thrombin, pre-thrombin, pro-thrombin, von Willebrand's factor, alpha,-antitrypsin, plasminogen activators, Factor VII, Factor VIII, Factor IX, Factor X and Factor XIII, nerve growth factor, LACI, platelet-derived endothelial cell growth factor (PD-ECGF), glucose oxidase, serum cholinesterase, inter-alpha trypsin inhibitor, antithrombin III, apo-lipoprotein species, Protein C, Protein S, or a variant or fragment of any of the above, or a fusion of albumin and any of the above.

47. The host cell of claim 32 wherein the protein product of choice comprises the sequence of albumin or a variant or fragment thereof.

48. The host cell of claim 32 wherein the protein product of choice comprises the sequence of transferrin family member, or a variant or fragment thereof.

49. The host cell of claim 32 wherein the protein product of choice comprises a fusion protein.

50. The host cell of claim 32 comprising an exogenous polynucleotide sequence that encodes the protein product of choice.

51. The host cell of claim 50 wherein the exogenous polynucleotide is integrated into the chromosome of the host cell.

52. The host cell of claim 50 wherein the exogenous polynucleotide is present in the host cell as part of a replicable vector.

53. A method for producing a protein product of choice, the method comprising:
(a) providing the host cell of claim 50; and
(b) growing the host cell;
thereby to produce a cell culture or recombinant organism comprising an increased level of the protein product of choice compared to the level of production of the protein product of choice achieved by growing, under the same conditions, the same host cell that has not been genetically modified to cause over-expression of one or more helper proteins.

54. The method of claim 53 wherein the step of growing the host cell involves culturing the host cell in a culture medium.

55. The method of claim 53 further comprising the step of purifying the thus expressed protein product of choice from the cultured host cell, recombinant organism or culture medium.

56. Method of preparing a the host cell of claim 32, by transformation of a host cell with a polynucleotide, wherein the polynucleotide comprises a sequence encoding a helper protein selected from the list comprising
(a) a chaperone selected from a DnaJ-like protein an Hsp70 family protein, and SIL1, and wherein the DnaJ-like protein is not SCJ1; and
(b) a protein involved in the formation of disulphide bonds in other proteins selected from ERO1, ERV2, EUG1, MPD1, MPD2, EPS1 and PDI1.


SEQUENCE LISTING (195 sequences)

SEQ ID NO: 1 - protein, 645 aa, Saccharomyces cerevisiae
MILISGYCLLVYSVILPVLISASKLCDLAELQRLNKNLKVDTESLPKYQWIAGQLEQNCMTADPASENMSDVIQLANQIYYKIGLIQLSNDQHLRAINTFEKIVFNETYKGSFGKLAEKRLQELYVDFGMWDKVHQKDDQYAKYLSLNETIRNKISSKDVSVEEDISELLRITPYDVNVLSTHIDVLFHKLAEEIDVSLAAAIILDYETILDKHLASLSIDTRLSIHYVISVLQTFVLNSDASFNIRKCLSIDMDYDKCKKLSLTISKLNKVNPSKRQILDPATYAFENKKFRSWDRIIEFYLKDKKPFITPMKILNKDTNFKNNYFFLEEIIKQLIEDVQLSRPLAKNLFEDPPITDGFVKPKSYYHTDYLVYIDSILCQASSMSPDVKRAKLAAPFCKKSLRHSLTLETWKHYQDAKSEQKPLPETVLSDVWNSNPHLLMYMVNSILNKSRSKPHSQFKKQLYDQINKFFQDNGLSESTNPYVMKNFRLLQKQLQTYKEHKHRNFNQQYFQQQQQQQQHQRHQAPPAAPNYDPKKDYYKILGVSPSASSKEIRKAYLNLTKKYHPDKIKANHNDKQESIHETMSQINEAYETLSDDDKRKEYDLSRSNPRRNTFPQGPRQNNMFKNPGSGFPFGNGFKMNFGL

SEQ ID NO: 2 - DNA, 1938 bp, Saccharomyces cerevisiae
atgatactgatctcgggatactgtcttttagtgtatagcgttattttgccagtactgatatcggcttctaagttatgtgatttggctgagttacaacgattgaacaagaatttaaaagtagacactgaatccttgccaaaataccaatggatcgctgggcagttggaacaaaactgcatgactgcggatccagcaagtgaaaatatgtcagacgtaattcaactagccaatcaaatatactacaaaattgggctgatccaattatccaacgatcaacatctaagagctattaacacatttgaaaaaatcgtttttaatgaaacttacaaaggttcttttgggaagctggcggaaaagaggctacaagagctgtatgtcgattttgggatgtgggacaaggtgcatcagaaggatgatcagtatgcgaaatatctgtccttgaatgaaaccatcagaaacaaaatatcatccaaagacgtttctgtggaggaagatatttctgagctgctacgcataacgccgtacgatgttaacgtcctctccacgcacatcgatgttctttttcacaaactagctgaagaaattgacgtttcgttagctgctgctatcattttggattacgaaacaatcctcgacaagcatttggctagcttaagcatagatacaagactttcgattcattatgtcatatctgttttacagacctttgtacttaactcagatgcgtcgttcaatataagaaaatgcctttccattgatatggactatgataaatgtaaaaaactaagcctgactatttccaaattgaacaaggtgaatccatcaaaaagacagatcctggatccagcaacatatgcatttgagaacaaaaagtttagaagttgggatagaattattgaattttatttgaaggataagaagccatttattacaccaatgaaaattcttaacaaagatacaaactttaaaaacaactacttctttttagaggaaattatcaaacaattgatagaagacgttcaactgtcgagacctttggcaaaaaatttattcgaagatcccccaataaccgatggttttgtcaaaccaaaatcatactatcataccgattatctagtatacattgattccattctttgtcaggcttctagcatgagtccggacgtcaagagagctaaactggctgcgccgttctgtaaaaagagtttgaggcattcactaacactagaaacatggaaacactatcaggatgctaagtccgagcaaaaacctttacctgagacggtattgagtgatgtatggaattccaatcctcatttgctgatgtatatggtaaactcaatacttaataaaagtaggtctaaacctcattcacagttcaaaaagcaattatatgaccagataaacaaatttttccaagataacggcctctcagagtcgaccaatccatacgtgatgaagaacttccgattattacagaaacaattacaaacctataaagagcataaacatcggaatttcaaccagcaatatttccaacaacaacaacagcagcaacaacaccaacgacatcaagcacccccagcagcgcctaactacgacccaaaaaaggactattataaaattcttggagtatcgcctagtgctagttcgaaagaaataaggaaagcatatttaaatttaaccaaaaaataccacccagacaaaataaaggccaaccataacgacaaacaagaatcaattcacgaaactatgtcacaaatcaatgaagcgtacgaaacattaagtgatgacgataaaaggaaggaatacgatctttccagatcaaacccccgccgcaacacttttcctcaggggcctaggcaaaataacatgttcaaaaatccaggaagtggcttcccattcggaaatggctttaaaatgaattttgggctttga

SEQ ID NO: 3 - protein, 881 aa, Saccharomyces cerevisiae
MRNVLRLLFLTAFVAIGSLAAVLGVDYGQQNIKAIVVSPQAPLELVLTPEAKRKEISGLSIKRLPGYGKDDPNGIERIYGSAVGSLATRFPQNTLLHLKPLLGKSLEDETTVTLYSKQHPGLEMVSTNRSTIAFLVDNVEYPLEELVAMNVQEIANRANSLLKDRDARTEDFVNKMSFTIPDFFDQHQRKALLDASSITTGIEETYLVSEGMSVAVNFVLKQRQFPPGEQQHYIVYDMGSGSIKASMFSILQPEDTTQPVTIEFEGYGYNPHLGGAKFTMDIGSLIENKFLETHPAIRTDELHANPKALAKINQAAEKAKLILSANSEASINIESLINDIDFRTSITRQEFEEFIADSLLDIVKPINDAVTKQFGGYGTNLPEINGVILAGGSSRIPIVQDQLIKLVSEEKVLRNVNADESAVNGVVMRGIKLSNSFKTKPLNVVDRSVNTYSFKLSNESELYDVFTRGSAYPNKTSILTNTTDSIPNNFTIDLFENGKLFETITVNSGAIKNSYSSDKCSSGVAYNITFDLSSDRLFSIQEVNCICQSENDIGNSKQIKNKGSRLAFTSEDVEIKRLSPSERSRLHEHIKLLDKQDKERFQFQENLNVLESNLYDARNLLMDDEVMQNGPKSQVEELSEMVKVYLDWLEDASFDTDPEDIVSRIREIGILKKKIELYMDSAKEPLNSQQFKGMLEEGHKLLQAIETHKNTVEEFLSQFETEFADTIDNVREEFKKIKQPAYVSKALSTWEETLTSFKNSISEIEKFLAKNLFGEDLREHLFEIKLQFDMYRTKLEEKLRLIKSGDESRLNEIKKLHLRNFRLQKRKEEKLKRKLEQEKSRNNNETESTVINSADDKTTIVNDKTTESNPSSEEDILHDEL

SEQ ID NO: 4 - DNA, 2646 bp, Saccharomyces cerevisiae
atgcgaaacgttttaaggcttttatttttaacagcttttgttgctatagggtctttagcagccgttttaggtgttgattacggtcagcaaaatatcaaggccattgtggtttctccgcaagccccattagaacttgtgctcacaccagaggcaaaacggaaggagatatctggtctttcgataaaaagattaccaggttatggaaaggatgatccgaatgggattgaaagaatctacggttccgctgttggcagtttagcaacaaggtttccccaaaacacattgttgcatttgaaaccgctacttgggaaatcactagaagatgaaaccactgtaactttgtattcaaaacaacaccccggtttagaaatggtatcaacaaatagaagtaccatagcctttttagttgataatgtggaatatccattggaagagttagtggcaatgaatgtccaagagattgccaatagagccaattcactgttgaaggatagagatgcaagaactgaggactttgtaaacaagatgagttttacaattcctgacttttttgaccaacatcaaaggaaagcacttttagatgccagttcaataaccacaggaatcgaagagacatatctggttagtgaagggatgtctgttgcagttaactttgtattaaagcagcgccaatttccaccaggtgaacagcagcattatatcgtatatgacatggggagcggttctattaaggcctcaatgttctctatattgcagccggaggacactactcagcccgttacaatagaatttgaaggatatgggtataatccacatctaggtggtgcaaagtttacaatggatattggcagtttgatagagaataagtttttggaaacacacccagccataagaactgatgaattgcacgctaatcccaaggccttagcaaaaatcaaccaagcagcagagaaggcaaagttaattttaagcgccaattctgaggcaagtattaacatagaatcactgatcaacgatattgatttccgtacttctataactagacaggaattcgaagaatttattgcagactcgttattggacattgtcaaacccataaatgacgctgttacaaaacaattcggtggctatggaacaaatttacctgagataaatggggtcattttggcgggaggctcttcccgaattcccattgtgcaggatcaattaatcaaactcgtatccgaagaaaaagtgttgagaaatgtcaatgctgatgaatcagctgtgaatggtgttgttatgagagggatcaagttatctaattcgtttaagaccaagccgttaaatgttgttgaccgttctgtaaatacttattcattcaaattatcaaacgaatctgaactgtatgatgtgttcacgcgcggaagtgcttatccaaacaaaacatctattttgacaaacacgactgattcgattcctaataattttaccattgacttatttgagaatggtaaattgttcgaaactatcacagttaattcaggagctataaagaattcatattcctctgataagtgctcgtcaggagttgcgtataacattactttcgacttgtccagtgatagattattctctattcaagaggttaactgcatttgtcagagcgaaaatgacataggtaactccaagcaaattaagaacaaaggcagccgtttggcttttacttctgaggatgttgagatcaaaaggctttctccttcagaacgttcgcgtttgcatgagcatatcaagttgctcgataaacaggataaggaaagatttcaattccaagaaaatttaaacgttcttgaaagtaacttgtatgatgctagaaacctgctaatggatgatgaagttatgcaaaatggaccaaaatcccaagtagaagagttatcggagatggttaaagtatatttggattggctcgaagatgcatcctttgatactgaccctgaggatatagttagcagaattagagaaattggaatattaaaaaagaaaatagaactttacatggattctgcaaaggaacctttgaactctcaacaatttaaaggaatgcttgaagaaggccataagttacttcaggctatagaaacccataagaataccgttgaagaatttttgagtcaatttgaaaccgagtttgcggataccatagataatgttagagaagaatttaaaaagattaagcaaccagcgtatgtgtcgaaggcgttatctacatgggaggaaaccttaacctcttttaaaaattccattagcgaaatagagaagttcctggcaaaaaacctatttggcgaagaccttcgtgaacatttatttgaaatcaaattacaatttgatatgtatcgtacgaaactagaggaaaaactgcgtttaataaaaagcggtgatgaaagtcgcttaaatgaaataaagaagttacatttaagaaacttccgcctacaaaagagaaaggaggaaaagttgaaaagaaagcttgaacaggaaaaaagcagaaacaacaatgaaacagaatcgacagtaatcaactcggctgacgataaaactactattgtcaatgacaagaccaccgagtcgaatccaagttctgaggaagacattttgcatgatgaattatag

SEQ ID NO: 5 - protein, 377 aa, Saccharomyces cerevisiae
MIPKLYIHLILSLLLLPLILAQDYYAILEIDKDATEKEIKSAYRQLSKKYHPDKNAGSEEAHQKFIEVGEAYDVLSDPEKKKIYDQFGADAVKNGGGGGGPGGPGAGGFHDPFDIFERMFQGGHGGPGGGFGQRQRQRGPMIKVQEKLSLKQFYSGSSIEFTLNLNDECDACHGSGSADGKLAQCPDCQGRGVIIQVLRMGIMTQQIQQMCGRCGGTGQIIKNECKTCHGKKVTKKNKFFHVDVPPGAPRNYMDTRVGEAEKGPDFDAGDLVIEFKEKDTENMGYRRRGDNLYRTEVLSAAEALYGGWQRTIEFLDENKPVKLSRPAHVVVSNGEVEVVKGFGMPKGSKGYGDLYIDYVVVMPKTFKSGQNMLKDEL

SEQ ID NO: 6 - DNA, 1134 bp, Saccharomyces cerevisiae
atgattccaaaattatatatacatttgatactatctttattgttgttgccgctaattttggcgcaggattattatgcaatactagagatagacaaagatgccactgagaaggaaatcaaatcagcgtacagacaattgtctaagaagtaccatccggataaaaatgctgggagcgaagaagcccatcaaaaattcattgaagtcggcgaggcatacgatgtattgagcgatcctgaaaagaaaaagatttatgaccagtttggtgcagatgctgtaaagaatggcggtggcggtggcggtccaggaggccctggcgcaggtggattccacgatccgtttgacatattcgaacggatgtttcaaggaggtcatggaggtcctggcggcggatttggccagagacagaggcagcgtggtccaatgatcaaggtccaggaaaaactatctttaaagcagttttattccgggtcctcgatagaatttactttaaacctaaacgatgaatgtgatgcatgccatggtagtggctctgcagatggtaagctggcccaatgtcccgattgtcaaggtcgtggggttataatacaagtgctgcgcatgggtattatgacgcagcagattcaacagatgtgtggtaggtgtggtggtacgggacaaattatcaaaaatgaatgcaaaacatgtcacggcaaaaaagttaccaaaaagaacaagttcttccacgttgacgttccaccaggcgcaccaagaaactacatggacacaagagtcggcgaggctgaaaaagggcctgactttgacgccggtgacttggtcatagaattcaaggaaaaggatactgagaacatgggttacagaagaagaggcgacaatctgtacagaacagaagttctttctgctgcggaagcgctatacggcggatggcaaagaacgatagaattccttgatgagaacaagcccgttaagttatctagacccgctcatgtagttgtctccaatggcgaagttgaagtcgtgaagggattcggcatgcccaagggtagcaagggttacggtgatttgtacatagactacgtcgttgtcatgccaaagactttcaaatctgggcaaaatatgctcaaagatgagttgtag

SEQ ID NO: 7 - protein, 682 aa, Saccharomyces cerevisiae
MFFNRLSAGKLLVPLSVVLYALFVVILPLQNSFHSSNVLVRGADDVENYGTVIGIDLGTTYSCVAVMKNGKTEILANEQGNRITPSYVAFTDDERLIGDAAKNQVAANPQNTIFDIKRLIGLKYNDRSVQKDIKHLPFNVVNKDGKPAVEVSVKGEKKVFTPEEISGMILGKMKQIAEDYLGTKVTHAVVTVPAYFNDAQRQATKDAGTIAGLNVLRIVNEPTAAAIAYGLDKSDKEHQIIVYDLGGGTFDVSLLSIENGVFEVQATSGDTHLGGEDFDYKIVRQLIKAFKKKHGIDVSDNNKALAKLKREAEKAKRALSSQMSTRIEIDSFVDGIDLSETLTRAKFEELNLDLFKKTLKPVEKVLQDSGLEKKDVDDIVLVGGSTRIPKVQQLLESYFDGKKASKGINPDEAVAYGAAVQAGVLSGEEGVEDIVLLDVNALTLGIETTGGVMTPLIKRNTAIPTKKSQIFSTAVDNQPTVMIKVYEGERAMSKDNNLLGKFELTGIPPAPRGVPQIEVTFALDANGILKVSATDKGTGKSESITITNDKGRLTQEEIDRMVEEAEKFASEDASIKAKVESRNKLENYAHSLKNQVNGDLGEKLEEEDKETLLDAANDVLEWLDDNFETAIAEDFDEKFESLSKVAYPITSKLYGGADGSGAADYDDEDEDDDGDYFEHDEL

SEQ ID NO: 8 - DNA, 2049 bp, Saccharomyces cerevisiae
atgtttttcaacagactaagcgctggcaagctgctggtaccactctccgtggtcctgtacgcccttttcgtggtaatattacctttacagaattctttccactcctccaatgttttagttagaggtgccgatgatgtagaaaactacggaactgttatcggtattgacttaggtactacttattcctgtgttgctgtgatgaaaaatggtaagactgaaattcttgctaatgagcaaggtaacagaatcaccccatcttacgtggcattcaccgatgatgaaagattgattggtgatgctgcaaagaaccaagttgctgccaatcctcaaaacaccatcttcgacattaagagattgatcggtttgaaatataacgacagatctgttcagaaggatatcaagcacttgccatttaatgtggttaataaagatgggaagcccgctgtagaagtaagtgtcaaaggagaaaagaaggtttttactccagaagaaatttctggtatgatcttgggtaagatgaaacaaattgccgaagattatttaggcactaaggttacccatgctgtcgttactgttcctgcttatttcaatgacgcgcaaagacaagccaccaaggatgctggtaccatcgctggtttgaacgttttgagaattgttaatgaaccaaccgcagccgccattgcctacggtttggataaatctgataaggaacatcaaattattgtttatgatttgggtggtggtactttcgatgtctctctattgtctattgaaaacggtgttttcgaagtccaagccacttctggtgatactcatttaggtggtgaagattttgactataagatcgttcgtcaattgataaaagctttcaagaagaagcatggtattgatgtgtctgacaacaacaaggccctagctaaattgaagagagaagctgaaaaggctaaacgtgccttgtccagccaaatgtccacccgtattgaaattgactccttcgttgatggtatcgacttaagtgaaaccttgaccagagctaagtttgaggaattaaacctagatctattcaagaagaccttgaagcctgtcgagaaggttttgcaagattctggtttggaaaagaaggatgttgatgatatcgttttggttggtggttctactagaattccaaaggtccaacaattgttagaatcatactttgatggtaagaaggcctccaagggtattaacccagatgaagctgttgcatacggtgcagccgttcaagctggtgtcttatccggtgaagaaggtgtcgaagatattgttttattggatgtcaacgctttgactcttggtattgaaaccactggtggtgtcatgactccattaattaagagaaatactgctattcctacaaagaaatcccaaattttctctactgccgttgacaaccaaccaaccgttatgatcaaggtatacgagggtgaaagagccatgtctaaggacaacaatctattaggtaagtttgaattaaccggcattccaccagcaccaagaggtgtacctcaaattgaagtcacatttgcacttgacgctaatggtattctgaaggtgtctgccacagataagggaactggtaaatccgaatctatcaccatcactaacgataaaggtagattaacccaagaagagattgatagaatggttgaagaggctgaaaaattcgcttctgaagacgcttctatcaaggccaaggttgaatctagaaacaaattagaaaactacgctcactctttgaaaaaccaagttaatggtgacctaggtgaaaaattggaagaagaagacaaggaaaccttattagatgctgctaacgatgttttagaatggttagatgataactttgaaaccgccattgctgaagactttgatgaaaagttcgaatctttgtccaaggtcgcttatccaattacttctaagttgtacggaggtgctgatggttctggtgccgctgattatgacgacgaagatgaagatgacgatggtgattatttcgaacacgacgaattgtag

SEQ ID NO: 9 - protein, 421 aa, Saccharomyces cerevisiae
MVRILPIILSALSSKLVASTILHSSIHSVPSGGEIISAEDLKELEISGNSICVDNRCYPKIFEPRHDWQPILPGQELPGGLDIRINMDTGLKEAKLNDEKNVGDNGSHELIVSSEDMKASPGDYEFSSDFKEMRNIIDSNPTLSSQDIARLEDSFDRIMEFAHDYKHGYKIITHEFALLANLSLNENLPLTLRELSTRVITSCLRNNPPVVEFINESFPNFKSKIMAALSNLNDSNHRSSNILIKRYLSILNELPVTSEDLPIYSTVVLQNVYERNNKDKQLQIKVLELISKILKADMYENDDTNLILFKRNAENWSSNLQEWANEFQEMVQNKSIDELHTRTFFDTLYNLKKIFKSDITINKGFLNWLAQQCKARQSNLDNGLQERDTEQDSFDKKLIDSRHLIFGNPMAHRIKNFRDEL

SEQ ID NO: 10 - DNA, 1266 bp, Saccharomyces cerevisiae
atggtccggattcttcccataattttgagcgccctatcttcgaaattagtggcgagtacaatattgcattcatccatacactcagtgccatctggaggcgaaatcatatctgcagaagatcttaaagaacttgaaatttcagggaattcgatctgcgttgataatcgttgctatcctaagatatttgaaccaagacacgattggcagcccatactgccaggtcaagaactccccggtggtttggacattagaataaacatggacacaggtttaaaagaggcaaaactaaatgatgagaagaatgtcggtgataatggtagccatgagttaattgtatcttcagaagacatgaaagcatcgcctggtgactatgaattttccagtgatttcaaagaaatgagaaacatcatagattctaacccgactttatcttcacaggacattgccagattggaggatagttttgatagaataatggaatttgcgcatgattacaagcacggctacaaaattattacccatgaattcgccctcttggccaaccttagtctcaatgaaaatttgccgttaacattgagagagctcagtactagagtcattaccagctgcttgagaaacaatcctcctgtagtcgagttcattaatgaaagttttccaaattttaaaagcaaaatcatggccgctctgtcaaatttgaatgattctaaccacagatcctctaatatcctaataaaaagatacttgtccattttaaacgaattacctgtcacatccgaagatcttcctatatactctacggttgttttacaaaatgtatatgaaagaaacaacaaggacaaacagttacaaataaaagtcctggagttgatcagcaaaattttgaaggccgacatgtacgaaaatgacgatacaaatctaattttgttcaaaagaaatgctgagaattggtcgtcaaatctgcaagagtgggcaaacgagttccaagagatggtccagaacaaaagtatagatgaactacatacaagaacgttttttgacaccctttacaacttgaagaaaattttcaaaagtgacatcacgatcaacaaagggtttttgaattggttagcgcaacaatgtaaagccaggcaatctaacttggacaatgggctccaagagagagatactgaacaagactcatttgataagaaacttatcgacagcagacacttgatctttggcaaccccatggctcatagaataaaaaatttcagagatgaactctga

SEQ ID NO: 11 - protein, 135 aa, Saccharomyces cerevisiae
MMFNIYLFVTFFSTILAGSLSDLEIGIIKRIPVEDCLIKAMPGDKVKVHYTGSLLESGTVFDSSYSRGSPIAFELGVGRVIKGWDQGVAGMCVGEKRKLQIPSSLAYGERGVPGVIPPSADLVFDVELVDVKSAA

SEQ ID NO: 12 - DNA, 408 bp, Saccharomyces cerevisiae
atgatgtttaatatttaccttttcgtcacttttttttccaccattcttgcaggttccctgtcagatttggaaatcggtattatcaagagaataccggtagaagattgcttaattaaggcaatgccaggtgataaagttaaggttcattatacaggatctttattagaatcgggaactgtatttgactcaagttattcaagaggctctcctatcgcttttgaacttggcgttggcagagtaattaaaggttgggatcaaggtgttgccggcatgtgcgttggcgaaaaaagaaagctgcaaattccaagttctttggcctacggagaaagaggtgtcccaggcgtcattcctccaagtgctgatttggtgtttgatgtcgaattggtagacgtgaaatcagccgcctag

SEQ ID NO: 13 - protein, 642 aa, Saccharomyces cerevisiae
MSKAVGIDLGTTYSCVAHFANDRVDIIANDQGNRTTPSFVAFTDTERLIGDAAKNQAAMNPSNTVFDAKRLIGRNFNDPEVQADMKHFPFKLIDVDGKPQIQVEFKGETKNFTPEQISSMVLGKMKETAESYLGAKVNDAVVTVPAYFNDSQRQATKDAGTIAGLNVLRIINEPTAAAIAYGLDKKGKEEHVLIFDLGGGTFDVSLLFIEDGIFEVKATAGDTHLGGEDFDNRLVNHFIQEFKRKNKKDLSTNQRALRRLRTACERAKRTLSSSAQTSVEIDSLFEGIDFYTSITRARFEELCADLFRSTLDPVEKVLRDAKLDKSQVDEIVLVGGSTRIPKVQKLVTDYFNGKEPNRSINPDEAVAYGAAVQAAILTGDESSKTQDLLLLDVAPLSLGIETAGGVMTKLIPRNSTISTKKFEIFSTYADNQPGVLIQVFEGERAKTKDNNLLGKFELSGIPPAPRGVPQIEVTFDVDSNGILNVSAVEKGTGKSNKITITNDKGRLSKEDIEKMVAEAEKFKEEDEKESQRIASKNQLESIAYSLKNTISEAGDKLEQADKDTVTKKAEETISWLDSNTTASKEEFDDKLKELQDIANPIMSKLYQAGGAPGGAAGGAPGGFPGGAPPAPEAEGPTVEEVD

SEQ ID NO: 14 - DNA, 1929 bp, Saccharomyces cerevisiae
atgtcaaaagctgtcggtattgatttaggtacaacatactcgtgtgttgctcactttgctaatgatcgtgtggacattattgccaacgatcaaggtaacagaaccactccatcttttgtcgctttcactgacactgaaagattgattggtgatgctgctaagaatcaagctgctatgaatccttcgaataccgttttcgacgctaagcgtttgatcggtagaaacttcaacgacccagaagtgcaggctgacatgaagcacttcccattcaagttgatcgatgttgacggtaagcctcaaattcaagttgaatttaagggtgaaaccaagaactttaccccagaacaaatctcctccatggtcttgggtaagatgaaggaaactgccgaatcttacttgggagccaaggtcaatgacgctgtcgtcactgtcccagcttacttcaacgattctcaaagacaagctaccaaggatgctggtaccattgctggtttgaatgtcttgcgtattattaacgaacctaccgccgctgccattgcttacggtttggacaagaagggtaaggaagaacacgtcttgattttcgacttgggtggtggtactttcgatgtctctttgttgttcattgaagacggtatctttgaagttaaggccaccgctggtgacacccatttgggtggtgaagattttgacaacagattggtcaaccacttcatccaagaattcaagagaaagaacaagaaggacttgtctaccaaccaaagagctttgagaagattaagaaccgcttgtgaaagagccaagagaactttgtcttcctccgctcaaacttccgttgaaattgactctttgttcgaaggtatcgatttctacacttccatcaccagagccagattcgaagaattgtgtgctgacttgttcagatctactttggacccagttgaaaaggtcttgagagatgctaaattggacaaatctcaagtcgatgaaattgtcttggtcggtggttctaccagaattccaaaggtccaaaaattggtcactgactacttcaacggtaaggaaccaaacagatctatcaacccagatgaagctgttgcttacggtgctgctgttcaagctgctattttgactggtgacgaatcttccaagactcaagatctattgttgttggatgtcgctccattatccttgggtattgaaactgctggtggtgtcatgaccaagttgattccaagaaactctaccatttcaacaaagaagttcgagatcttttccacttatgctgataaccaaccaggtgtcttgattcaagtctttgaaggtgaaagagccaagactaaggacaacaacttgttgggtaagttcgaattgagtggtattccaccagctccaagaggtgtcccacaaattgaagtcactttcgatgtcgactctaacggtattttgaatgtttccgccgtcgaaaagggtactggtaagtctaacaagatcactattaccaacgacaagggtagattgtccaaggaagatatcgaaaagatggttgctgaagccgaaaaattcaaggaagaagatgaaaaggaatctcaaagaattgcttccaagaaccaattggaatccattgcttactctttgaagaacaccatttctgaagctggtgacaaattggaacaagctgacaaggacaccgtcaccaagaaggctgaagagactatttcttggttagacagcaacaccactgccagcaaggaagaattcgatgacaagttgaaggagttgcaagacattgccaacccaatcatgtctaagttgtaccaagctggtggtgctccaggtggcgctgcaggtggtgctccaggcggtttcccaggtggtgctcctccagctccagaggctgaaggtccaaccgttgaagaagttgattaa

SEQ ID NO: 15 - protein, 639 aa, Saccharomyces cerevisiae
MSKAVGIDLGTTYSCVAHFSNDRVDIIANDQGNRTTPSFVGFTDTERLIGDAAKNQAAMNPANTVFDAKRLIGRNFNDPEVQGDMKHFPFKLIDVDGKPQIQVEFKGETKNFTPEQISSMVLGKMKETAESYLGAKVNDAVVTVPAYFNDSQRQATKDAGTIAGLNVLRIINEPTAAAIAYGLDKKGKEEHVLIFDLGGGTFDVSLLSIEDGIFEVKATAGDTHLGGEDFDNRLVNHFIQEFKRKNKKDLSTNQRALRRLRTACERAKRTLSSSAQTSVEIDSLFEGIDFYTSITRARFEELCADLFRSTLDPVEKVLRDAKLDKSQVDEIVLVGGSTRIPKVQKLVTDYFNGKEPNRSINPDEAVAYGAAVQAAILTGDESSKTQDLLLLDVAPLSLGIETAGGVMTKLIPRNSTIPTKKSEVFSTYADNQPGVLIQVFEGERAKTKDNNLLGKFELSGIPPAPRGVPQIEVTFDVDSNGILNVSAVEKGTGKSNKITITNDKGRLSKEDIEKMVAEAEKFKEEDEKESQRIASKNQLESIAYSLKNTISEAGDKLEQADKDAVTKKAEETIAWLDSNTTATKEEFDDQLKELQEVANPIMSKLYQAGGAPEGAAPGGFPGGAPPAPEAEGPTVEEVD

SEQ ID NO: 16 - DNA, 1920 bp, Saccharomyces cerevisiae
atgtctaaagctgtcggtattgatttaggtactacctactcctgtgttgctcacttctctaatgatcgtgttgacattattgccaacgaccaaggtaacagaaccactccatctttcgttggtttcactgatactgaaagattgattggtgacgctgctaagaaccaagctgctatgaacccagctaacactgttttcgacgctaagcgtttgatcggtagaaacttcaatgacccagaagtccaaggtgatatgaagcacttcccattcaagttgatcgatgttgacggtaagccacaaattcaagttgaatttaagggtgaaaccaagaactttaccccagaacaaatctcctccatggtcttgggtaagatgaaggaaactgccgaatcttacttgggtgccaaggtcaatgacgctgtcgtcactgtcccagcttacttcaacgattctcaaagacaagctaccaaggatgctggtaccattgctggtttgaatgtcttgcgtattattaacgaacctaccgccgctgccattgcttacggtttggacaagaagggtaaggaagaacacgtcttgattttcgacttgggtggtggtactttcgatgtctctttgttgtccattgaagacggtatctttgaagttaaggccaccgctggtgacacccatttgggtggtgaagattttgacaacagattggtcaaccacttcatccaagaattcaagagaaagaacaagaaggacttgtctaccaaccaaagagctttgagaagattaagaactgcttgtgaaagagccaagagaactttgtcttcctccgctcaaacttccgttgaaattgactctttgttcgaaggtatcgatttctacacttccatcaccagagccagattcgaagaattgtgtgctgacttgttcagatctactttggacccagttgaaaaggtcttgagagatgctaaattggataaatctcaagtcgatgaaattgtcttggtcggtggttctaccagaattccaaaggtccaaaaattggtcactgactacttcaacggtaaggaaccaaacagatctatcaacccagatgaagctgttgcttacggtgctgctgttcaagctgctattttgactggtgacgaatcttccaagactcaagatctattgttgttggatgtcgctccattatccttgggtattgaaactgctggtggtgtcatgaccaagttgattccaagaaactctaccattccaactaagaaatccgaagttttctctacttatgctgacaaccaaccaggtgtcttgattcaagtctttgaaggtgaaagagccaagactaaggacaacaacttgttgggtaagttcgaattgagtggtattccaccagctccaagaggtgtcccacaaattgaagtcactttcgatgtcgactctaacggtattttgaatgtttccgccgtcgaaaagggtactggtaagtctaacaagatcactattaccaacgacaagggtagattgtccaaggaagatatcgaaaagatggttgctgaagccgaaaaattcaaggaagaagatgaaaaggaatctcaaagaattgcttccaagaaccaattggaatccattgcttactctttgaagaacaccatttctgaagctggtgacaagctagagcaagctgacaaggacgctgtcactaagaaggctgaagaaactattgcttggttagacagcaacaccactgctaccaaggaagaattcgatgaccaattgaaggaattgcaagaggttgccaacccaatcatgtctaaattgtaccaagctggtggtgctccagaaggcgcagctccaggtggtttcccaggtggtgctcctccagctccagaagctgaaggtccaactgtcgaagaagttgattaa

SEQ ID NO: 17 - protein, 649 aa, Saccharomyces cerevisiae
MSRAVGIDLGTTYSCVAHFSNDRVEIIANDQGNRTTPSYVAFTDTERLIGDAAKNQAAINPHNTVFDAKRLIGRKFDDPEVTTDAKHFPFKVISRDGKPVVQVEYKGETKTFTPEEISSMVLSKMKETAENYLGTTVNDAVVTVPAYFNDSQRQATKDAGTIAGMNVLRIINEPTAAAIAYGLDKKGRAEHNVLIFDLGGGTFDVSLLSIDEGVFEVKATAGDTHLGGEDFDNRLVNHLATEFKRKTKKDISNNQRSLRRLRTAAERAKRALSSSSQTSIEIDSLFEGMDFYTSLTRARFEELCADLFRSTLEPVEKVLKDSKLDKSQIDEIVLVGGSTRIPKIQKLVSDFFNGKEPNRSINPDEAVAYGAAVQAAILTGDQSTKTQDLLLLDVAPLSLGIETAGGIMTKLIPRNSTIPTKKSETFSTYADNQPGVLIQVFEGERTRTKDNNLLGKFELSGIPPAPRGVPQIDVTFDIDANGILNVSALEKGTGKSNKITITNDKGRLSKDDIDRMVSEAEKYRADDEREAERVQAKNQLESYAFTLKNTINEASFKEKVGEDDAKRLETASQETIDWLDASQAASTDEYKDRQKELEGIANPIMTKFYGAGAGAGPGAGESGGFPGSMPNSGATGGGEDTGPTVEEVD

SEQ ID NO: 18 - DNA, 1950 bp, Saccharomyces cerevisiae
atgtctagagcagttggtattgatttgggaacaacttactcgtgtgttgctcatttttccaatgatagggtagagataattgcaaatgatcaaggtaataggaccactccatcgtatgtggctttcacagacaccgaaagattaattggtgacgccgccaaaaatcaagctgcaatcaatcctcataatacagtttttgatgcaaagcggttaattggtcgtaaatttgatgatcctgaagtgacgacagatgccaagcacttccctttcaaagttatatccagagatggtaaacctgtagtgcaagtagaatataagggtgaaacgaaaacatttacgcctgaggaaatttcttccatggttttaagcaaaatgaaggaaactgctgagaactatttgggaactacggtcaatgatgctgttgtaactgttcctgcatatttcaatgattctcaaagacaagccactaaggatgcaggaactattgcagggatgaacgttttacgtattatcaatgaacccactgcagcagcaattgcttatggcttggataagaaaggcagggctgagcacaatgtcctgatttttgatttgggtggtggtacttttgacgtctctttactttcaattgatgagggtgtttttgaggttaaggctaccgcaggagacactcatttaggtggtgaagattttgataataggttggtgaaccatttagccactgaattcaaaaggaaaacgaaaaaggacatctctaataatcaaagatcgttaagaagattgagaactgcggcagaaagagctaagagagcgctttcttcctcatctcaaacctcgatcgagatcgattctttatttgaaggtatggatttctacacttcgttaacaagggcaaggtttgaagagctatgtgctgatttattcagatccacattggaaccagtagaaaaggttcttaaagattcgaagctggacaagtcccaaattgatgagattgtgttagtcggtggatctaccagaatcccaaagattcagaaattagtttctgacttcttcaatggcaaagagcctaatcgttctatcaacccggatgaggctgttgcttatggtgcagccgttcaagctgccattttaaccggcgatcaatcaacaaagacacaagatttactattattggatgttgcgccattgtccctaggaattgaaactgcaggcggcataatgactaagctaattcctagaaactcaacgattccaacaaagaaatcggaaaccttctctacctatgcagataatcaacctggtgttttaattcaagtctttgaaggtgaaagaacaagaacaaaggataataacttacttggtaaattcgaattaagtggcattccgcctgctcccagaggtgtgcctcaaattgatgttacctttgatatcgacgctaatggtattcttaatgtgtctgctttggaaaagggtactggtaagagtaacaaaatcacgatcactaacgataaaggtaggctctcgaaggatgatattgataggatggtttctgaagctgaaaaatatagggctgacgatgaaagggaggcagaacgagttcaggctaagaatcagcttgaatcgtatgcatttactttgaagaataccataaacgaagcaagtttcaaagagaaagtaggtgaagatgatgcaaagagattagaaacagcgtctcaggaaaccattgactggttagatgcatcgcaggcagcctctacggacgaatataaggatagacaaaaggagttggaaggcattgccaatccaataatgacgaaattttacggtgctggtgccggcgcaggtcctggagcgggggaatccggtggattccccggatccatgcccaactcgggtgctacgggaggtggagaagatacaggtccaacagtggaagaggttgattga

SEQ ID NO: 19 - protein, 642 aa, Saccharomyces cerevisiae
MSKAVGIDLGTTYSCVAHFANDRVEIIANDQGNRTTPSYVAFTDTERLIGDAAKNQAAMNPHNTVFDAKRLIGRKFDDPEVTNDAKHYPFKVIDKGGKPVVQVEYKGETKTFTPEEISSMILTKMKETAENFLGTEVKDAVVTVPAYFNDSQRQATKDAGTIAGLNVLRIINEPTAAAIAYGLDKKSQKEHNVLIFDLGGGTFDVSLLSIDEGVFEVKATAGDTHLGGEDFDSRLVNFLAEEFKRKNKKDLTTNQRSLRRLRTAAERAKRTLSSSAQTSIEIDSLFEGIDFYTSITRARFEELCADLFRSTLEPVEKVLADSKLDKSQIDEIVLVGGSTRIPKVQKLVSDFFNGKEPNRSINPDEAVAYGAAVQAAILTGDQSSTTQDLLLLDVAPLSLGIETAGGIMTKLIPRNSTIPTKKSEVFSTYADNQPGVLIQVFEGERTRTKDNNLLGKFELSGIPPAPRGVPQIEVTFDIDANGILNVSAVEKGTGKSNKITITNDKGRLSKEDIDKMVAEAEKFKAEDEQEAQRVQAKNQLESYAFTLKNSVSENNFKEKVGEEDARKLEAAAQDAINWLDASQAASTEEYKERQKELEGVANPIMSKFYGAAGGAPGAGPVPGAGAGPTGAPDNGPTVEEVD

SEQ ID NO: 20 - DNA, 1929 bp, Saccharomyces cerevisiae
atgtcaaaagctgttggtattgatttaggtacaacctattcatgtgttgctcattttgcaaacgatagggttgaaattatcgctaacgatcaaggtaatagaacgacgccttcttatgtggcttttactgacacagaaaggctaattggtgacgctgcgaagaatcaagctgcgatgaacccacataatacagtattcgatgctaagcgtctgatcggacgtaaattcgatgatccagaagtgacgaacgatgctaagcattacccattcaaagtgattgacaagggaggtaaaccggtagtgcaagtggaatataaaggcgagacaaagacatttactccagaagaaatttcctcaatgatcttgacaaagatgaaggagactgctgagaactttttaggaacagaagtgaaagatgctgtagtaacggttccagcctatttcaacgattcacaaaggcaagcaacaaaagatgccggtacaatcgcgggcttgaacgttcttcgtatcattaatgaacctacagctgccgctattgcgtatgggctggacaagaaatcgcagaaggagcacaacgtcttgatctttgatttaggtggtggtacttttgatgtctctctgctatccatagatgaaggtgtctttgaggttaaggctactgctggtgacactcacttgggtggtgaagatttcgatagtaggctggttaactttctagccgaggagttcaaaagaaaaaataaaaaggatctaacaactaaccaaaggtccctaaggaggttaaggaccgccgctgaaagggccaagagaactctgtcttcgtctgctcagacatctatagaaatagattcattatttgagggtatcgatttctatacttccattacaagggcaagatttgaagaattatgtgctgatttgtttagatctacattggagccagtggaaaaagttttggctgattcaaaattagataagtcacaaattgatgaaattgtacttgttggtggttcaacaagaattccaaaagtacaaaaactggtttctgattttttcaatggtaaagaaccaaaccgttcgattaaccctgatgaggccgtcgcttatggtgctgccgtacaggctgccatcttaacgggtgaccagtcgtcgacgacccaagatttactgttgctggatgttgcaccattatctctaggtattgaaactgcaggtggtattatgacaaagttgatcccaagaaattcgactatcccaacaaaaaaatcggaagtgttttccacctacgctgacaaccaacctggtgtgttgatacaagtttttgagggtgaaaggacaaggacaaaagacaacaatctactgggtaaatttgagttgagcggtattccacccgctccaagaggcgtaccacaaattgaagttacatttgatatcgatgcaaatggtattctgaacgtatctgccgttgaaaaaggtactggtaaatctaacaagattacaattactaacgataagggaagattatcgaaggaagatatcgataaaatggttgctgaggcagaaaagttcaaggccgaagatgaacaagaagctcaacgtgttcaagctaagaatcagctagaatcgtacgcgtttactttgaaaaattctgtgagcgaaaataacttcaaggagaaggtgggtgaagaggatgccaggaaattggaagccgccgcccaagatgctataaattggttagatgcttcgcaagcggcctccaccgaggaatacaaggaaaggcaaaaggaactagaaggtgttgcaaaccccattatgagtaaattttacggagctgcaggtggtgccccaggagcaggcccagttccgggtgctggagcaggccccactggagcaccagacaacggcccaacggttgaagaggttgattag

SEQ ID NO: 21 - protein, 693 aa, Saccharomyces cerevisiae
MSTPFGLDLGNNNSVLAVARNRGIDIVVNEVSNRSTPSVVGFGPKNRYLGETGKNKQTSNIKNTVANLKRIIGLDYHHPDFEQESKHFTSKLVELDDKKTGAEVRFAGEKHVFSATQLAAMFIDKVKDTVKQDTKANITDVCIAVPPWYTEEQRYNIADAARIAGLNPVRIVNDVTAAGVSYGIFKTDLPEGEEKPRIVAFVDIGHSSYTCSIMAFKKGQLKVLGTACDKHFGGRDFDLAITEHFADEFKTKYKIDIRENPKAYNRILTAAEKLKKVLSANTNAPFSVESVMNDVDVSSQLSREELEELVKPLLERVTEPVTKALAQAKLSAEEVDFVEIIGGTTRIPTLKQSISEAFGKPLSTTLNQDEAIAKGAAFICAIHSPTLRVRPFKFEDIHPYSVSYSWDKQVEDEDHMEVFPAGSSFPSTKLITLNRTGDFSMAASYTDITQLPPNTPEQIANWEITGVQLPEGQDSVPVKLKLRCDPSGLHTIEEAYTIEDIEVEEPIPLPEDAPEDAEQEFKKVTKTVKKDDLTIVAHTFGLDAKKLNELIEKENEMLAQDKLVAETEDRKNTLEEYIYTLRGKLEEEYAPFASDAEKTKLQGMLNKAEEWLYDEGFDSIKAKYIAKYEELASLGNIIRGRYLAKEEEKKQAIRSKQEASQMAAMAEKLAAQRKAEAEKKEEKKDTEGDVDMD

SEQ ID NO: 22 - DNA, 2082 bp, Saccharomyces cerevisiae
atgagtactccatttggtttagatttaggtaacaataactctgtccttgccgttgctagaaacagaggtatcgacattgtcgttaatgaagtctctaaccgttccaccccatctgttgttggttttggtccaaagaacagatacttgggtgaaactggtaagaacaagcagacttccaacatcaagaacactgtcgccaacttgaaaagaattattggtttggattaccaccatccagatttcgagcaagaatctaagcacttcacctctaagttggttgaattggatgacaagaagactggtgccgaagttagattcgctggtgagaaacatgttttttcagctactcaactagctgccatgttcatcgacaaagtcaaggacaccgtcaagcaggacacaaaggcaaatattaccgatgtttgtattgctgtcccaccttggtacaccgaagaacaacgttacaacattgctgatgctgctagaattgctggtttgaaccctgttagaattgtcaacgacgttactgctgccggtgtttcttacggtatcttcaagactgatttgcctgaaggcgaagaaaagccaagaattgttgcctttgttgatattggtcactcttcctacacctgttctatcatggccttcaagaagggtcaattgaaagtcttaggaactgcctgcgacaagcattttggtggtagggacttcgatttggctataacagaacatttcgccgatgagttcaaaactaaatacaagattgacatcagagaaaatccaaaggcttacaacagaattctaactgctgctgaaaagttgaagaaagttttgtctgctaatactaatgccccattctctgttgaatccgtcatgaacgacgttgatgtttcctctcaattatctcgtgaagaattagaagaattggtcaagccattgttggaacgtgttactgaaccagttaccaaagctttagctcaagccaaattatctgctgaagaagttgattttgttgaaattattggtggtactactcgtatcccaacattgaaacaatccatttctgaagccttcggcaagccattgtccaccactttgaaccaagatgaagccatcgccaagggtgccgcctttatttgcgccattcactctccaactctaagagttagaccattcaagtttgaggatatccatccttactctgtctcttactcttgggacaagcaagttgaggacgaagaccacatggaagttttcccagctggttcatccttcccatctactaaattgatcactttgaaccgtacgggtgacttttcaatggctgctagctacactgacatcacacagttaccaccaaacactccagaacaaatcgctaactgggagatcactggtgttcaattaccagaaggtcaagactctgttcctgttaagttaaagttgagatgcgacccctctggtttacacacaattgaagaggcttacactattgaagatattgaagttgaagaacctattccattaccagaagatgctccagaagatgctgagcaagaatttaagaaggttactaaaactgtaaagaaggatgacttaaccatcgttgcacacacctttggcctagacgctaaaaagttgaatgaattaattgaaaaagaaaatgaaatgcttgctcaagataagctagttgctgagacagaagaccgtaagaacactcttgaagagtacatctacacattgcgtggtaagttggaagaagagtatgctccatttgcttccgatgctgaaaagacgaagttacaaggtatgttaaacaaggccgaagagtggttatacgatgaaggtttcgattccatcaaagctaagtacattgccaaatacgaagaattggcttctctaggtaacattattagaggtagatacttggctaaagaagaagaaaagaagcaagctataagatctaagcaagaagcatcccaaatggctgctatggctgaaaagttggctgctcaaagaaaggcagaagctgaaaagaaggaagaaaagaaggacactgaaggtgatgttgacatggactaa

SEQ ID NO: 23 - protein, 693 aa, Saccharomyces cerevisiae
MSTPFGLDLGNNNSVLAVARNRGIDVVVNEVSNRSTPSLVGFGPRNRYLGESGKTKQTSNVKNTVENLKRIIGLKFKDPEFDIENKFFTSKLVQLKNGKVGVEVEFGGKTHVFSATQLTAMFIDKVKHTVQEETKSSITDVCLAVPVWYSEEQRYNIADAARIAGLNPVRIVNDVTAAAVSYGVFKNDLPGPEEKPRIIGLVDIGHSTYTCSIMAFRKGEMKVLGTAYDKHFGGRDFDRAITEHFADQFKDKYKIDIRKNPKAYNRILIAAEKLKKVLSANTTAPFSVESVMDDIDVSSQLSREELEELVEPLLKRVTYPITNALAQAKLTVNDIDFVEIIGGTTRIPVLKKSISDVFGKPLSSTLNQDEAVAKGAAFICAIHSPTLRVRPFKFEDIDPYSVSYTWDKQVDDEDRLEVFPANSSYPSTKLITLHRTGDFSMKAVYTHPSKLPKGTSTTIAKWSFTGVKVPKDQDFIPVKVKLRCDPSGLHIIENAYTTEDITVQEPVPLPEDAPEDAEPQFKEVTKTIKKDVLGMTAKTFALNPVELNDLIEKENELRNQDKLVAETEDRKNALEEYIYTLRAKLDDEYSDFASDAEKEKLKNMLATTENWLYGDGDDSTKAKYIAKYEELASLGNIIRGRYLAKEEEKRQALRANQETSKMNDIAEKLAEQRRARAASDDSDDNNDENMDLD

SEQ ID NO: 24 - DNA, 2082 bp, Saccharomyces cerevisiae
atgagcactccatttggcttagatttaggtaacaataactcagtactagcagttgccagaaataggggtattgatgtcgttgtcaatgaagtttctaataggtctacaccatccttggtcggctttggccccagaaataggtacttaggtgaatctggtaaaactaagcaaacatcgaatgttaaaaacactgtggaaaacttgaaaagaatcattggactaaagttcaaagaccctgaatttgatatcgagaataagttcttcacttcgaaattggtacagctaaaaaatggtaaagttggtgtggaagtggagttcggcggtaaaacacacgtattttcagctactcaactgactgctatgttcattgataaggtgaagcacaccgttcaagaggaaacgaagtcatcaattaccgatgtctgcctcgcagttcctgtatggtattcggaagaacaacgttataacatagccgatgctgccagaattgcaggattaaatcctgtaaggattgtcaacgatgtgactgcagccgccgtttcgtacggcgtcttcaagaatgatctgccaggtcctgaagaaaagccaagaatcattggcttagtggacattgggcattctacctacacctgttctattatggctttccgcaaaggcgaaatgaaagtattaggtactgcttatgacaagcactttggtggtagagatttcgatcgcgcaatcacagaacattttgctgatcagtttaaggacaagtacaagattgacattaggaaaaatccgaaagcttataacagaattttaatcgctgctgaaaaattaaaaaaagtgctttctgcgaacactactgcccccttctccgttgaatctgttatggatgatatcgacgtttcctctcaattgagccgtgaagagctggaagaattagtagagcccttgttgaagcgtgtgacgtatccaatcaccaatgcattggctcaagctaaattaactgtcaatgatattgacttcgtagaaataattggtggtacaacccgtatcccagttttaaagaagtcaatttctgatgtttttggaaaacctttgtcatctactttaaatcaagacgaagctgtggccaagggggccgctttcatatgtgccattcactctccaactttaagggtcaggccgtttaaatttgaagatattgatccgtattcagtgtcatacacttgggataagcaggtcgatgacgaagaccgtttggaagtattccctgctaattcatcatatccatcaactaaactaattactttacatcgtactggagatttcagcatgaaagcggtgtacactcatccttcgaaactgccaaaaggtacttccaccactattgcaaaatggagcttcactggggtcaaggttcctaaagatcaagattttattcctgtaaaggtcaagttaagatgcgatccttccggcttgcatattatcgagaacgcttacacaacggaagatattacggttcaagagccagtgcctttaccggaagacgcaccagaagatgccgagccccagtttaaagaagttactaaaacaattaagaaagatgtgctaggtatgactgcaaaaacattcgcgctaaacccggttgagttgaacgatctaattgaaaaagagaatgaattaagaaaccaggataagttagttgccgaaaccgaggatcgcaaaaatgcccttgaagagtatatttatacccttcgtgccaaactcgatgatgaatactccgattttgcgtctgacgcagaaaaagaaaagctaaaaaacatgttagccactactgaaaattggttatatggtgatggtgacgattctaccaaggcaaaatacattgctaaatatgaggagctggcatcgttggggaatattattagaggtagatatttagcaaaggaggaagaaaaaagacaagcactcagagcgaatcaagaaacttctaaaatgaatgatattgctgaaaaattggctgagcaaagaagggcacgcgctgcaagtgatgatagcgatgacaacaatgatgaaaacatggaccttgattaa

SEQ ID NO: 25 - protein, 613 aa, Saccharomyces cerevisiae
MAEGVFQGAIGIDLGTTYSCVATYESSVEIIANEQGNRVTPSFVAFTPEERLIGDAAKNQAALNPRNTVFDAKRLIGRRFDDESVQKDMKTWPFKVIDVDGNPVIEVQYLEETKTFSPQEISAMVLTKMKEIAEAKIGKKVEKAVITVPAYFNDAQRQATKDAGAISGLNVLRIINEPTAAAIAYGLGAGKSEKERHVLIFDLGGGTFDVSLLHIAGGVYTVKSTSGNTHLGGQDFDTNLLEHFKAEFKKKTGLDISDDARALRRLRTAAERAKRTLSSVTQTTVEVDSLFDGEDFESSLTRARFEDLNAALFKSTLEPVEQVLKDAKISKSQIDEVVLVGGSTRIPKVQKLLSDFFDGKQLEKSINPDEAVAYGAAVQGAILTGQSTSDETKDLLLLDVAPLSLGVGMQGDMFGIVVPRNTTVPTIKRRTFTTCADNQTTVQFPVYQGERVNCKENTLLGEFDLKNIPMMPAGEPVLEAIFEVDANGILKVTAVEKSTGKSSNITISNAVGRLSSEEIEKMVNQAEEFKAADEAFAKKHEARQRLESYVASIEQTVTDPVLSSKLKRGSKSKIEAALSDALAALQIEDPSADELRKAEVGLKRVVTKAMSSR

SEQ ID NO: 26 - DNA, 1842 bp, Saccharomyces cerevisiae
atggctgaaggtgttttccaaggtgctatcggtatcgatttaggtacaacctactcttgtgttgctacttacgaatcctccgttgaaattattgccaacgaacaaggtaacagagtcaccccatctttcgttgctttcactccagaagaaagattgattggtgatgctgccaagaaccaagctgctttgaacccaagaaacactgtcttcgatgctaagcgtttgattggtagaagattcgacgacgaatctgttcaaaaggacatgaagacctggcctttcaaggttatcgacgtcgatggtaacccagtcatcgaagtccaatacttggaagaaaccaagactttctccccacaagaaatttccgctatggttttgaccaagatgaaggaaattgctgaagctaagattggtaagaaggttgaaaaggccgtcattactgtcccagcttactttaacgacgctcaaagacaagctaccaaggatgccggtgccatttctggtttgaacgttttgcgtatcatcaacgaacctactgccgctgctattgcttacggtctaggtgctggtaagtccgaaaaggaaagacatgttttgattttcgatttgggtggtggtactttcgatgtttccttgttgcacattgctggtggtgtttacactgttaaatctacttccggtaacactcacttgggtggtcaagatttcgacaccaacttgttggaacacttcaaggctgaattcaagaagaagactggtttggacatctccgacgatgccagagctttgagaagattgagaactgctgctgaaagagctaagagaaccttatcttctgtcactcaaactaccgttgaagttgactctttgtttgacggtgaagatttcgaatcctctttgactagagctagatttgaagacttgaacgccgcattgttcaagtctactttggaacctgttgaacaagttttgaaggatgctaagatctctaagtctcaaatcgacgaagttgtcttggttggtggttccaccagaattccaaaggtccaaaagttgttgtctgacttctttgacggtaagcaattggaaaaatctattaacccagatgaagctgttgcttacggtgctgctgttcaaggtgctatcttgaccggccaatccacatctgacgaaaccaaggacttgttgttgttagatgttgctccattatctctaggtgttggtatgcaaggtgacatgttcggtatcgttgttccaagaaacactactgttccaaccatcaagagaagaacctttactacatgtgctgacaaccaaaccaccgttcaattcccagtctaccaaggtgaacgtgttaactgtaaagaaaacactttgttgggtgaattcgacttgaagaacatcccaatgatgccagctggtgaaccagtcttggaagctatcttcgaagttgatgctaacggtatcttgaaggttactgccgtcgaaaagtctaccggtaagtcttctaacatcactatctctaacgctgttggtagattgtcttctgaagaaattgaaaagatggttaaccaagctgaagagttcaaggctgccgatgaagcttttgccaagaagcacgaagctagacaaagattggaatcctacgttgcctccatcgaacaaactgtcactgacccagtcttgtcttctaaattgaagagaggttccaagtccaagattgaagctgctttgtccgatgctttggctgctttgcaaatcgaagacccatctgctgatgaattgagaaaggctgaagttggtttgaagagagttgtcaccaaggccatgtcttctcgttaa

SEQ ID NO: 27 - protein, 613 aa, Saccharomyces cerevisiae
MAEGVFQGAIGIDLGTTYSCVATYESSVEIIANEQGNRVTPSFVAFTPQERLIGDAAKNQAALNPRNTVFDAKRLIGRRFDDESVQKDMKTWPFKVIDVDGNPVIEVQYLEETKTFSPQEISAMVLTKMKEIAEAKIGKKVEKAVITVPAYFNDAQRQATKDAGAISGLNVLRIINEPTAAAIAYGLGAGKSEKERHVLIFDLGGGTFDVSLLHIAGGVYTVKSTSGNTHLGGQDFDTNLLEHFKAEFKKKTGLDISDDARALRRLRTAAERAKRTLSSVTQTTVEVDSLFDGEDFESSLTRARFEDLNAALFKSTLEPVEQVLKDAKISKSQIDEVVLVGGSTRIPKVQKLLSDFFDGKQLEKSINPDEAVAYGAAVQGAILTGQSTSDETKDLLLLDVAPLSLGVGMQGDIFGIVVPRNTTVPTIKRRTFTTVSDNQTTVQFPVYQGERVNCKENTLLGEFDLKNIPMMPAGEPVLEAIFEVDANGILKVTAVEKSTGKSSNITISNAVGRLSSEEIEKMVNQAEEFKAADEAFAKKHEARQRLESYVASIEQTVTDPVLSSKLKRGSKSKIEAALSDALAALQIEDPSADELRKAEVGLKRVVTKAMSSR

SEQ ID NO: 28 - DNA, 1842 bp, Saccharomyces cerevisiae
atggctgaaggtgttttccaaggtgctatcggtatcgatttaggtacaacatactcttgtgttgctacttatgaatcttccgttgaaattattgccaacgaacaaggtaacagagttactccatctttcgttgccttcaccccacaggaaagattgatcggtgatgctgccaagaaccaagctgctttgaacccaagaaacactgtttttgatgctaagcgtttgattggtagaagattcgacgacgagtctgtccaaaaggacatgaagacctggcctttcaaggttatcgacgtcgatggtaacccagtcattgaagtccaatacttggaagaaaccaagactttctccccacaagaaatttccgctatggtcttgaccaagatgaaggaaattgctgaagctaagattggtaagaaggttgaaaaggctgtcattactgtcccagcttactttaacgatgcccaaagacaagctaccaaggatgccggtgccatttctggtttgaacgttttgcgtatcatcaacgaacctactgccgctgctattgcttacggtctaggtgctggtaagtccgaaaaggaaagacatgttttgattttcgatttgggtggtggtactttcgatgtttccttgttgcacattgctggtggtgtttacactgttaaatctacttccggtaacactcacttgggtggtcaagatttcgacaccaacttgttggaacacttcaaggctgaattcaagaagaagactggtttggacatctccgacgatgccagagctttgagaagattgagaactgctgctgaaagagctaagagaaccttatcttctgtcactcaaactaccgttgaagttgactctttgtttgacggtgaagatttcgaatcctctttgactagagctagatttgaagacttgaacgccgcattgttcaagtctactttggaacctgttgaacaagttttgaaggatgctaagatctctaagtctcaaatcgacgaagttgtcttggttggtggttctaccagaattccaaaggtccaaaagttgttgtctgacttctttgacggtaagcaattggaaaaatctattaacccagatgaagctgttgcttacggtgctgctgttcaaggtgctatcttgactggccaatccacatctgacgaaaccaaggacttgttgttgttagatgttgctccattatctctaggtgttggtatgcaaggtgacattttcggtattgttgtcccaagaaacacaactgttccaaccatcaagagaagaaccttcacaactgtcagtgacaaccaaaccaccgttcaattcccagtctaccaaggtgaacgtgtcaactgtaaagaaaacactttgttgggtgaattcgacttgaagaacatcccaatgatgccagctggtgaaccagtcttggaagctatcttcgaagttgatgctaacggtatcttgaaggttactgccgtcgaaaagtctaccggtaagtcttctaacatcactatctccaacgctgtcggtagattgtcttctgaagaaattgaaaagatggttaaccaagccgaagagttcaaggctgctgatgaagcttttgctaagaagcacgaagctagacaaagactagaatcctacgtcgcttccatcgaacaaaccgtcactgacccagtcttgtcttctaaattgaagagaggttccaagtccaagatcgaagctgctttgtccgatgctttggctgctttgcaaatcgaagacccatccgctgatgagttgagaaaggcagaagttggtttgaagagagttgtcaccaaggccatgtcttctcgttaa

SEQ ID NO: 29 - protein, 644 aa, Saccharomyces cerevisiae
MLPSWKAFKAHNILRILTRFQSTKIPDAVIGIDLGTTNSAVAIMEGKVPRIIENAEGSRTTPSVVAFTKDGERLVGEPAKRQSVINSENTLFATKRLIGRRFEDAEVQRDINQVPFKIVKHSNGDAWVEARNRTYSPAQIGGFILNKMKETAEAYLAKSVKNAVVTVPAYFNDAQRQATKDAGQIIGLNVLRVVNEPTAAALAYGLDKSEPKVIAVFDLGGGTFDISILDIDNGIFEVKSTNGDTHLGGEDFDIYLLQEIISHFKKETGIDLSNDRMAVQRIREAAEKAKIELSSTLSTEINLPFITADAAGPKHIRMPFSRVQLENITAPLIDRTVDPVKKALKDARITASDISDVLLVGGMSRMPKVADTVKKLFGKDASKAVNPDEAVALGAAIQAAVLSGEVTDVLLLDVTPLSLGIETLGGVFTKLIPRNSTIPNKKSQIFSTAASGQTSVEVKVFQGERELVKDNKLIGNFTLAGIPPAPKGTPQIEVTFDIDANGIINVSAKDLASHKDSSITVAGASGLSDTEIDRMVNEAERYKNQDRARRNAIETANKADQLANDTENSIKEFEGKLDKTDSQRLKDQISSLRELVSRSQAGDEVNDDDVGTKIDNLRTSSMKLFEQLYKNSDNPETKNGRENK

SEQ ID NO: 30 - DNA, 1935 bp, Saccharomyces cerevisiae
atgttaccatcatggaaagcctttaaagcacataatatacttcgtattctgacccgtttccagtcaaccaaaattccagatgcagttatcggtattgatttaggtactaccaattctgcggtagctattatggaaggtaaagttccgagaattatcgaaaatgcagaaggctcaagaactactccgtctgtagtggctttcactaaagacggagaacgtttagttggtgagccagccaaacgacaatccgtcataaactcagaaaacactttgtttgctactaagcgtttaatcggccgccgtttcgaggacgctgaagtccaaagagatattaatcaggttcctttcaaaatcgtcaagcattctaatggagatgcctgggtagaggctagaaacagaacgtactcccccgcccaaataggaggttttatcttaaataaaatgaaggaaacagcggaggcttacttagcgaagagcgtcaaaaatgctgttgtcaccgttcctgcttacttcaatgatgcccaaagacaagctactaaagacgcaggacaaattattgggcttaatgtattacgtgttgtcaacgaaccaacagctgctgccctagcttacggtctagataaatcagagccaaaagtcattgctgttttcgacttgggcggtggtactttcgatatttcaatcctggacatcgataacggtatctttgaggttaaatctaccaatggtgacacccatttgggtggcgaagattttgacatttatttgttgcaagaaattatttctcatttcaagaaagaaaccggtatcgatttgagtaatgaccgtatggctgtccaaagaataagagaagccgctgaaaaggctaaaatcgaactgtcttctacactctctacagaaataaacttgcctttcataactgctgatgctgcaggcccaaagcatattcgtatgcccttttctagggttcagcttgagaatataaccgccccattgattgatagaacggttgatcctgtcaaaaaagcactgaaagacgcaagaattaccgcctcagatatatcggatgttttattagttggtggtatgtcaaggatgcccaaggttgcagatactgtaaagaaattattcggtaaggatgcatcaaaagctgttaaccctgatgaagcagtcgctttaggggccgctatacaggctgcggtcttgtctggtgaagttaccgatgttttgttgctagatgtcactcccctatcattgggtattgaaactttaggaggagtttttacaaaattaatcccaagaaattctacaattcccaataagaaatctcaaattttttcaactgcggcatcaggtcaaacatcggtggaagttaaagttttccaaggtgagagggagttagtcaaggataacaaattaataggtaattttactcttgcgggcattcctccagctccaaaaggtaccccacaaattgaagtcacttttgatatcgatgcgaacggcatcatcaacgtttcagcaaaagatctcgccagccacaaagactcttccatcactgttgccggagcgtctgggctatctgatacggagattgatcgaatggttaatgaagcggaaagatataaaaatcaggatagagccagaaggaatgccatcgaaaccgctaacaaagctgaccagctagctaatgacacagaaaattccattaaggaattcgaaggtaagctagataaaactgattctcaaagactaaaagatcaaatttcatccttaagggaattggtttctcggagtcaagctggagatgaggttaatgatgacgatgttggaacaaaaattgacaatttgcgaacttcatcgatgaaactttttgaacagttatacaagaacagtgacaatcctgaaactaagaacgggagagaaaataaataa

SEQ ID NO: 31 - protein, 511 aa, Saccharomyces cerevisiae
MAFQQGVLSRCSGVFRHHVGHSRHINNILYRHAIAFASIAPRIPKSSFHTSAIRNNEAFKDPYDTLGLKKSATGAEIKKAYYKLAKKYHPDINKEPDAEKKFHDLQNAYEILSDETKRQQYDQFGPAAFGGGGAAGGAGGGSGSPFGSQFHDFSGFTSAGGSPFGGINFEDLFGAAFGGGGRGSGGASRSSSMFRQYRGDPIEIVHKVSFKDAVFGSKNVQLRFSALDPCSTCSGTGMKPNTHKVSCSTCHGTGTTVHIRGGFQMMSTCPTCNGEGTMKRPQDNCTKCHGEGVQVNRAKTITVDLPHGLQDGDVVRIPGQGSYPDIAVEADLKDSVKLSRGDILVRIRVDKDPNFSIKNKYDIWYDKEIPITTAALGGTVTIPTVEGQKIRIKVAPGTQYNQVISIPNMGVPKTSTIRGDMKVQYKIVVKKPQSLAEKCLWEALADVTNDDMAKKTMQPGTAAGTAINEEILKKQKQEEEKHAKKDDDNTLKRLENFITNTFRKIKGDKKN

SEQ ID NO: 32 - DNA, 1536 bp, Saccharomyces cerevisiae
atggctttccaacaaggtgtattgtcaaggtgttccggtgtctttagacaccatgtgggacattctcgccatatcaataatattctttatagacatgccatcgcgtttgcatccatcgctccacgaataccaaaatctagcttccatacttctgcaatcagaaacaacgaagcattcaaggacccgtacgatactttaggcttgaagaaatctgctacaggtgcggaaatcaaaaaagcatactacaaactggcaaagaagtaccacccggatatcaacaaggaaccggatgctgagaagaaattccacgatttacagaacgcttatgaaattctgtcagacgaaacgaagaggcagcagtacgatcaatttgggcccgctgccttcggcggcggcggtgccgctggaggtgccggtggtggtagtggctctccctttggttcccaatttcatgatttctcaggattcaccagtgcaggcggctcgccatttggcggtatcaattttgaagacctgtttggtgctgcatttggtggtggtggccgcggtagcggtggcgcaagcaggtcgtcatctatgttcagacaatataggggcgacccaatcgagattgtccataaagtgtctttcaaggacgcagtgtttgggtccaagaacgttcagttaagattctctgcgctggacccttgtagtacctgttcagggacgggaatgaaaccaaacacgcataaggtcagttgtagcacttgtcacggaacaggaaccactgttcacattaggggcggatttcagatgatgtcgacttgtcctacttgcaacggtgaaggtaccatgaaacggcctcaggacaattgtaccaagtgccatggtgagggtgttcaggtcaacagggcaaagacaattacggtggacttgccacatggattacaggacggcgacgtggtcaggatccctggccaaggctcataccctgacatcgctgtagaggcggacttgaaagattcagtcaagttatcaagaggtgatattttggtgagaattcgtgtcgacaaggatcccaacttttcgataaagaacaagtacgatatttggtacgacaaggagattcctataaccacagctgcacttggtggtactgtcactatccccactgtggagggacaaaagatcaggataaaggtcgctccagggactcaatacaatcaagtgatatccattcctaacatgggtgttcctaaaacatcaaccattcgcggtgatatgaaagtccagtacaagatcgttgttaagaaaccgcaatcgctggcagaaaaatgcttgtgggaggcactggcagatgtcaccaacgatgacatggccaagaaaaccatgcaaccgggcacagccgcgggtacagccattaatgaagagatactgaagaaacaaaaacaagaagaggaaaaacacgcaaaaaaggatgacgacaacactttgaagagactagaaaatttcattaccaacacattcaggaagatcaaaggtgacaaaaaaaattaa

SEQ ID NO: 33 - protein, 146 aa, Saccharomyces cerevisiae
MVLPIIIGLGVTMVALSVKSGLNAWTVYKTLSPLTIAKLNNIRIENPTAGYRDALKFKSSLIDEELKNRLNQYQGGFAPRMTEPEALLILDISAREINHLDEKLLKKKHRKAMVRNHPDRGGSPYMAAKINEAKEVLERSVLLRKR

SEQ ID NO: 34 - DNA, 441 bp, Saccharomyces cerevisiae
atggttttgcctataataattggtttgggcgtgacaatggttgctctaagtgtcaagtctggtctcaatgcatggaccgtctacaagaccctgtcccctttaactattgcaaaactaaataacattcgcatagaaaacccgacggcgggctaccgcgatgcacttaagttcaaaagctcactgatagacgaagaactgaaaaatagattaaaccagtaccagggaggctttgcaccgcgaatgacagagcccgaagccttgctcatcttggatatctccgccagagagattaatcacttggatgaaaaattactgaaaaaaaagcacaggaaggctatggttcgtaaccacccagacagaggagggagtccctacatggcggccaagataaatgaggcgaaagaagttctcgaaagaagtgttttactaagaaagagataa

SEQ ID NO: 35 - protein, 563 aa, Saccharomyces cerevisiae
MRLRTAIATLCLTAFTSATSNNSYIATDQTQNAFNDTHFCKVDRNDHVSPSCNVTFNELNAINENIRDDLSALLKSDFFKYFRLDLYKQCSFWDANDGLCLNRACSVDVVEDWDTLPEYWQPEILGSFNNDTMKEADDSDDECKFLDQLCQTSKKPVDIEDTINYCDVNDFNGKNAVLIDLTANPERFTGYGGKQAGQIWSTIYQDNCFTIGETGESLAKDAFYRLVSGFHASIGTHLSKEYLNTKTGKWEPNLDLFMARIGNFPDRVTNMYFNYAVVAKALWKIQPYLPEFSFCDLVNKEIKNKMDNVISQLDTKIFNEDLVFANDLSLTLKDEFRSRFKNVTKIMDCVQCDRCRLWGKIQTTGYATALKILFEINDADEFTKQHIVGKLTKYELIALLQTFGRLSESIESVNMFEKMYGKRLNGSENRLSSFFQNNFFNILKEAGKSIRYTIENINSTKEGKKKTNNSQSHVFDDLKMPKAEIVPRPSNGTVNKWKKAWNTEVNNVLEAFRFIYRSYLDLPRNIWELSLMKVYKFWNKFIGVADYVSEETREPISYKLDIQ

SEQ ID NO: 36 - DNA, 1692 bp, Saccharomyces cerevisiae
atgagattaagaaccgccattgccacactgtgcctcacggcttttacatctgcaacttcaaacaatagctacatcgccaccgaccaaacacaaaatgcctttaatgacactcacttttgtaaggtcgacaggaatgatcacgttagtcccagttgtaacgtaacattcaatgaattaaatgccataaatgaaaacattagagatgatctttcggcgttattaaaatctgatttcttcaaatactttcggctggatttatacaagcaatgttcattttgggacgccaacgatggtctgtgcttaaaccgcgcttgctctgttgatgtcgtagaggactgggatacactgcctgagtactggcagcctgagatcttgggtagtttcaataatgatacaatgaaggaagcggatgatagcgatgacgaatgtaagttcttagatcaactatgtcaaaccagtaaaaaacctgtagatatcgaagacaccatcaactactgtgatgtaaatgactttaacggtaaaaacgccgttctgattgatttaacagcaaatccggaacgatttacaggttatggtggtaagcaagctggtcaaatttggtctactatctaccaagacaactgttttacaattggcgaaactggtgaatcattggccaaagatgcattttatagacttgtatccggtttccatgcctctatcggtactcacttatcaaaggaatatttgaacacgaaaactggtaaatgggagcccaatctggatttgtttatggcaagaatcgggaactttcctgatagagtgacaaacatgtatttcaattatgctgttgtagctaaggctctctggaaaattcaaccatatttaccagaattttcattctgtgatctagtcaataaagaaatcaaaaacaaaatggataacgttatttcccagctggacacaaaaatttttaacgaagacttagtttttgccaacgacctaagtttgactttgaaggacgaattcagatctcgcttcaagaatgtcacgaagattatggattgtgtgcaatgtgatagatgtagattgtggggcaaaattcaaactaccggttacgcaactgccttgaaaattttgtttgaaatcaacgacgctgatgaattcaccaaacaacatattgttggtaagttaaccaaatatgagttgattgcactattacagactttcggtagattatctgaatctattgaatctgttaacatgttcgaaaaaatgtacgggaaaaggttaaacggttctgaaaacaggttaagctcattcttccaaaataacttcttcaacattttgaaggaggcaggcaaatcgattcgttacaccatagagaacatcaattccactaaagaaggaaagaaaaagactaacaattctcaatcacatgtatttgatgatttaaaaatgcccaaagcagaaatagttccaaggccctctaacggtacagtaaataaatggaagaaagcttggaatactgaagttaacaacgttttagaagcattcagatttatttatagaagctatttggatttacccaggaacatctgggaattatctttgatgaaggtatacaaattttggaataaattcatcggtgttgctgattacgttagtgaggagacacgagagcctatttcctataagctagatatacaataa

SEQ ID NO: 37 - protein, 196 aa, Saccharomyces cerevisiae
MKQIVKRSHAIRIVAALGIIGLWMFFSSNELSIATPGLIKAKSGIDEVQGAAAEKNDARLKEIEKQTIMPLMGDDKVKKEVGRASWKYFHTLLARFPDEPTPEEREKLHTFIGLYAELYPCGECSYHFVKLIEKYPVQTSSRTAAAMWGCHIHNKVNEYLKKDIYDCATILEDYDCGCSDSDGKRVSLEKEAKQHG

SEQ ID NO: 38 - DNA, 591 bp, Saccharomyces cerevisiae
atgaaacagatagtcaaaagaagccatgccatcagaatagttgcagcattaggaatcataggcctgtggatgtttttctcgtctaatgaactatccatcgctacgccgggcctaatcaaggcgaagtctggtatagatgaagtgcaaggggcggctgctgagaagaacgacgctcggttgaaagagatcgagaagcaaaccattatgccattgatgggcgatgacaaggtgaagaaggaagtgggcagggcgtcgtggaagtacttccataccctgctggcccgttttccggacgagcctactcctgaagaaagagagaaactgcacacgtttattgggttgtatgcagaactctatccatgcggggaatgttcatatcactttgtaaagttgattgagaagtatcccgtacagacatctagcaggacggctgccgcaatgtggggatgccacattcacaacaaggtgaacgaatacctaaagaaagacatatatgactgtgctaccatcctggaggactacgattgtggatgtagtgacagcgacggtaaacgcgtgtctctcgagaaggaggctaaacagcacggttga

SEQ ID NO: 39 - protein, 517 aa, Saccharomyces cerevisiae
MQVTTRFISAIVSFCLFASFTLAENSARATPGSDLLVLTEKKFKSFIESHPLVLVEFFAPWCLHSQILRPHLEEAASILKEHNVPVVQIDCEANSMVCLQQTINTYPTLKIFKNGRIFDGQVYRGVKITDEITQYMIQLYEASVIYLNSEDEIQPYLENATLPVVINRGLTGLNETYQEVALDLAEDYVFLSLLDSEDKSLSIHLPNTTEPILFDGNVDSLVGNSVALTQWLKVVILPYFTDIEPDLFPKYISSNLPLAYFFYTSEEELEDYTDLFTQLGKENRGQINFIALNSTMFPHHVRFLNMREQFPLFAIHNMINNLKYGLPQLPEEEYAKLEKPQPLDRDMIVQLVKDYREGTAKPIVKSEEIPKEQKSNVYKIVGKTHDDIVHDDDKDVLVKYYATWCIHSKRFAPIYEEIANVLASDESVRDKILIAEVDSGANDILSFPVTGYPTIALYPAGNNSKPIIFNKIRNLEDVFEFIKESGTHHIDGQAIYDKLHQAKDSEVSTEDTVHDEL

SEQ ID NO: 40 - DNA, 1554 bp, Saccharomyces cerevisiae
atgcaagtgaccacaagatttatatctgcgatagtctcgttttgcctgtttgcttctttcacgttggctgaaaacagcgcaagagctacgccgggatcagatttactcgttctaacagagaagaaatttaaatcattcatcgaatctcatccgttagtcctcgtcgagttttttgctccatggtgtttgcattctcagatcttacgccctcacttagaagaggccgcctctattttaaaggagcataacgtcccagttgttcaaattgattgtgaggctaacagtatggtttgcctgcaacaaactataaatacctacccaaccttgaaaatctttaaaaatggtcgtatttttgatggtcaagtctatcgcggtgtcaagatcaccgatgaaatcactcagtacatgattcagctatacgaggcttctgtcatttatttaaattccgaagatgaaatccaaccatacttggaaaatgcaactttaccagtagtaataaacagaggcttgacaggcttgaatgaaacgtatcaagaagtcgcactggaccttgctgaggattacgtctttttatcccttctagattcagaagataagtcattatcaatccacttgccaaacactacagaaccaattctgtttgatggaaatgtagactctttggtcggaaattccgttgctctaactcagtggttaaaagtggtaattttaccttactttaccgacatcgaacctgatctcttccccaagtacatttctagcaatttgccgttggcttacttcttttatacttctgaggaagaattggaagattacactgatcttttcacgcagttaggtaaggaaaatcgtggccaaataaatttcattgcattaaactctacaatgttcccacaccacgttagattcctaaatatgagagaacagttcccattatttgctatccataatatgatcaataatctgaaatatggtttaccacaactaccagaagaagagtacgcgaaattagaaaaaccacaaccactagacagagatatgatcgttcagttggtaaaagattaccgtgaaggtactgccaagccaattgttaagtcagaagagattccaaaagaacaaaagtccaatgtttataaaatagttgggaagacacatgacgacattgttcatgatgatgacaaggatgtccttgtcaaatattacgcgacatggtgtattcatagtaaaaggtttgcgcctatttacgaagaaattgcaaatgtcttagcatctgatgaatctgttcgcgataaaatcttgatcgccgaagtagattcaggggcaaatgatatcttaagttttcctgtgacaggatatccaaccattgctttgtatcctgccggaaataactctaagcctattatcttcaataaaattagaaatttggaagatgttttcgaatttatcaaggaatcaggtacacatcacattgacggccaggcaatttatgataaattgcaccaggccaaggattctgaagtgtctactgaagataccgtacatgatgaattataa

SEQ ID NO: 41 - protein, 318 aa, Saccharomyces cerevisiae
MLFLNIIKLLLGLFIMNEVKAQNFYDSDPHISELTPKSFDKAIHNTNYTSLVEFYAPWCGHCKKLSSTFRKAAKRLDGVVQVAAVNCDLNKNKALCAKYDVNGFPTLMVFRPPKIDLSKPIDNAKKSFSAHANEVYSGARTLAPIVDFSLSRIRSYVKKFVRIDTLGSLLRKSPKLSVVLFSKQDKISPVYKSIALDWLGKFDFYSISNKKLKQLTDMNPTYEKTPEIFKYLQKVIPEQRQSDKSKLVVFDADKDKFWEYEGNSINKNDISKFLRDTFSITPNEGPFSRRSEYIAYLKTGKKPIKKNHSSSGNKHDEL

SEQ ID NO: 42 - DNA, 957 bp, Saccharomyces cerevisiae
atgttatttcttaatattattaagctccttttgggactttttattatgaatgaagtaaaggcgcaaaacttttacgattccgatcctcatatatcagagttaacgccaaaaagcttcgataaagcgatccataacacaaattacacatcattagtggaattttatgctccgtggtgcggccattgtaagaagctctctagtacgttccgcaaggcagcaaaaagattggatggtgtagtccaagttgctgctgtaaactgtgaccttaacaagaataaggctttgtgtgctaaatacgacgtaaacggatttcccacgttaatggtatttaggcccccaaaaattgacctatctaagccaatagataacgccaaaaaaagtttcagcgctcatgccaatgaagtgtactcaggtgcaagaactctcgcgcctattgttgatttttctctttcaagaataaggtcatatgtcaaaaagtttgtccgtatagatacacttggctctttacttagaaagtcacccaaactttccgtggtgttgttttccaaacaagacaaaatttcaccggtttataaaagcattgcccttgattggttaggaaagttcgatttttattcaatttcaaacaaaaaactcaagcaactaaccgatatgaacccaacatatgaaaaaactcctgagattttcaaatatttgcagaaggtcattcctgaacagcgacagagcgataaaagtaagcttgtcgtttttgatgcagacaaagataaattttgggagtatgaagggaactcaatcaacaaaaatgacatttccaaatttctgcgggacacttttagtattacccccaatgagggtccttttagtagacgttctgaatatattgcttacttaaaaactggcaagaagccaattaaaaagaaccattcctcctcaggaaacaagcacgacgaattgtag

SEQ ID NO: 43 - protein, 277 aa, Saccharomyces cerevisiae
MKLHGFLFSVLSTCVVILPALAYSEAVTMVKSIEQYFDICNRNDSYTMIKYYTSWCQHCKTLAPVYEELGELYAKKANKDDTPINFLEVNCEFFGPTLCTDLPGFPIIELVKPRTKPLVLPKLDWSSMKFHERLWQRIKTWFNNPKYQLDTSRVVRFEGSRNLKSLSNFIDTVRSKDTEERFIEHIFDDSRNCNEELRSQQLLCKAGKEYYSDTLSKLYGDVNGLEKERRRLEALIKQNGDDLSKEVKEKLKIIRLQLSLLSHIEDQLEDTSSHDEL

SEQ ID NO: 44 - DNA, 834 bp, Saccharomyces cerevisiae
atgaaattgcacggctttttattttccgtattatcaacatgcgtcgtcattttaccagcgttggcctacagtgaagctgtcacgatggtcaagtcgattgagcagtacttcgatatctgcaataggaatgattcttacacaatgataaaatactacacttcttggtgccaacattgtaaaactctggccccagtatacgaagagcttggtgagctatacgccaaaaaagctaataaagatgataccccaattaacttccttgaagttaactgtgaattcttcgggccaactttatgtaccgacttgcctggatttccaataattgaactggtcaaacctcgtactaagcccttagttcttccgaagctcgattggtcgtctatgaaatttcatgaaagactatggcaaagaatcaagacgtggttcaacaatcctaagtaccaactggatacgtctagggttgttcgttttgaagggagtaggaacctaaagagtttaagcaactttatcgatactgtaagaagtaaagatacagaagaaagattcatagaacatattttcgatgattctaggaattgcaatgaagaattacgttctcaacagcttctgtgtaaagctggtaaagaatactactctgatactttatctaaattatacggtgacgtgaatgggctggaaaaggaaaggcgaagactagaagctttaattaagcaaaatggagatgacttgagtaaagaagttaaagaaaaactgaaaatcattcgtctacaattgagcctattatcacacatagaagaccagttagaagataccagtagtcatgacgagctttga

SEQ ID NO: 45 - protein, 701 aa, Saccharomyces cerevisiae
MKMNLKRLVVTFFSCITFLLKFTIAAAEPPEGFPEPLNPTNFKEELSKGLHIIDFYSPYCPHCKHLAPVWMETWEEFKEESKTLNITFSQVNCIESADLCGDENIEYFPEIRLYNPSGYIKSFTETPRTKESLIAFARRESMDPNNLDTDLDSAKSESQYLEGFDFLELIAGKATRPHLVSFWPTKDMKNSDDSLEFKNCDKCHEFQRTWKIISRQLAVDDINTGHVNCESNPTICEELGFGDLVKITNHRADREPKVALVLPNKTSNNLFDYPNGYSAKSDGYVDFARRTFTNSKFPNITEGELEKKANRDIDFLQERGRVTNNDIHLVFSYDPETVVIEDFDILEYLIEPLSKIPNIYLHQIDKNLINLSRNLFGRMYEKINYDASQTQKVFNKEYFTMNTVTQLPTFFMFKDGDPISYVFPGYSTTEMRNIDAIMDWVKKYSNPLVTEVDSSNLKKLISFQTKSYSDLAIQLISSTDHKHIKGSNKLIKNLLLASWEYEHIRMENNFEEINERRARKADGIKKIKEKKAPANKIVDKMREEIPHMDQKKLLLGYLDISKEKNFFRKYGITGEYKIGDVIIIDKSNNYYYNKDNFGNSLTSNNPQLLREAFVSLNIPSKALYSSKLKGRLINSPFHNVLSFLDIIHGNGMPGYLIVIVLFIAILKGPSIYRRYKVRKHYRAKRNAVGILGNMEKKKNQD

SEQ ID NO: 46 - DNA, 2106 bp, Saccharomyces cerevisiae
atgaaaatgaatctgaaaaggctcgtagttaccttcttctcatgcatcacctttctgctgaaattcactatagccgccgctgaaccaccagagggctttccagagcccttaaatccaacaaacttcaaagaagagctatctaaggggctgcatattattgacttctatagtccatactgtccgcactgcaaacatttagcacctgtttggatggaaacatgggaggagtttaaagaggagagcaaaacactgaacataacattttcacaggttaactgcatcgagagcgccgatttgtgtggagatgaaaatattgaatacttccctgaaattagactttataacccctcaggatacatcaaatcgttcactgaaacaccgaggaccaaagaatcattaattgcatttgcacgcagggagtctatggacccaaataacctcgatactgatctggattctgctaaaagtgagagccagtatctcgaaggctttgattttctcgagctgatcgctggtaaggcgactaggccacatttggtttccttctggccaacaaaagatatgaaaaatagcgatgattcactagaattcaaaaactgtgacaaatgccatgaattccaaaggacttggaagatcatttcaagacagttagccgtggatgatatcaacacgggccacgttaattgcgaatctaatccaacaatctgtgaagaactgggctttggcgacttggtgaaaataaccaaccacagagccgatagagaacccaaggtagcattagtcctacccaataaaacctcaaataatttgttcgactatcccaatggctactcagcgaagtcagatggctatgtagattttgccaggaggacttttacaaacagtaaatttcccaatataacagaaggggagctcgaaaaaaaagcaaacagagacattgattttctgcaagaaaggggacgagtaactaataatgatatccatttagttttttcatatgaccccgaaactgttgttattgaagattttgacattttggagtatttaatcgagcctttgtcaaaaattccaaacatatatttgcaccaaattgacaagaatctaataaatttgtcacgtaatctttttggaagaatgtatgaaaagatcaactacgacgccagccaaactcaaaaggtttttaacaaagaatactttactatgaatacggttacgcaactcccaacttttttcatgtttaaagatggtgatcccatatcctatgttttccccggatactccacaacagaaatgagaaatattgatgccattatggattgggtaaaaaagtattctaatcccttagttaccgaagttgactcttctaatttgaaaaaattaatttccttccaaaccaagagctacagtgatttagcaattcagttaataagtagcactgaccacaaacatatcaaaggaagcaacaagcttattaaaaacttgctcctcgcaagttgggagtatgaacatattcggatggaaaataacttcgaagaaattaatgagagaagggcaaggaaagcagacgggatcaagaaaataaaggaaaaaaaggctccggctaacaaaattgttgataaaatgcgtgaagagattccccatatggatcaaaaaaaattgttattaggatatttagatatttcaaaggagaagaatttttttagaaaatatggtattactggagaatataaaattggtgatgtgattatcattgataaatcaaataattactactacaataaagataattttggcaactccttgacttctaacaaccctcaattgctgagagaagcattcgtgtccttaaatattccatcaaaagctctatacagctctaagttgaaggggagattgataaattctccattccataatgtcctcagtttcctagacataatccacgggaacggcatgcccggttacttaattgttattgttttgtttatcgcaatactcaaaggtccatctatttacagaagatacaaagtaaggaaacactatagggcgaaaaggaacgctgtcggtatcctaggaaatatggagaaaaaaaaaaatcaagattaa

SEQ ID NO: 47 - protein, 522 aa, Saccharomyces cerevisiae
MKFSAGAVLSWSSLLLASSVFAQQEAVAPEDSAVVKLATDSFNEYIQSHDLVLAEFFAPWCGHCKNMAPEYVKAAETLVEKNITLAQIDCTENQDLCMEHNIPGFPSLKIFKNSDVNNSIDYEGPRTAEAIVQFMIKQSQPAVAVVADLPAYLANETFVTPVIVQSGKIDADFNATFYSMANKHFNDYDFVSAENADDDFKLSIYLPSAMDEPVVYNGKKADIADADVFEKWLQVEALPYFGEIDGSVFAQYVESGLPLGYLFYNDEEELEEYKPLFTELAKKNRGLMNFVSIDARKFGRHAGNLNMKEQFPLFAIHDMTEDLKYGLPQLSEEAFDELSDKIVLESKAIESLVKDFLKGDASPIVKSQEIFENQDSSVFQLVGKNHDEIVNDPKKDVLVLYYAPWCGHCKRLAPTYQELADTYANATSDVLIAKLDHTENDVRGVVIEGYPTIVLYPGGKKSESVVYQGSRSLDSLFDFIKENGHFDVDGKALYEEAQEKAAEEADADAELADEEDAIHDEL

SEQ ID NO: 48 - protein, 530 aa, Saccharomyces cerevisiae
MKFSAGAVLSWSSLLLASSVFAQQEAVAPEDSAVVKLATDSFNEYIQSHDLVLAEFFAPWCGHCKNMAPEYVKAAETLVEKNITLAQIDCTENQDLCMEHNIPGFPSLKIFKNRDVNNSIDYEGPRTAEAIVQFMIKQSQPAVAVVADLPAYLANETFVTPVIVQSGKIDADFNATFYSMANKHFNDYDFVSAENADDDFKLSIYLPSAMDEPVVYNGKKADIADADVFEKWLQVEALPYFGEIDGSVFAQYVESGLPLGYLFYNDEEELEEYKPLFTELAKKNRGLMNFVSIDARKFGRHAGNLNMKEQFPLFAIHDMTEDLKYGLPQLSEEAFDELSDKIVLESKAIESLVKDFLKGDASPIVKSQEIFENQDSSVFQLVGKNHDEIVNDPKKDVLVLYYAPWCGHCKRLAPTYQELADTYANATSDVLIAKLDHTENDVRGVVIEGYPTIVLYPGGKKSESVVYQGSRSLDSLFDFIKENGHFDVDGKALYEEAQEKAAEEAEADAEAEADADAELADEEDAIHDEL

SEQ ID NO: 49 - protein, 211 aa, Saccharomyces cerevisiae
MDAVILNLLGDIPLVTRLWTIGCLVLSGLTSLRIVDPGKVVYSYDLVFKKGQYGRLLYSIFDYGAFNWISMINIFVSANHLSTLENSFNLRRKFCWIIFLLLVILVKMTSIEQPAASLGVLLHENLVYYELKKNGNQMNVRFFGAIDVSPSIFPIYMNAVMYFVYKRSWLEIAMNFMPGHVIYYMDDIIGKIYGIDLCKSPYDWFRNTETP

SEQ ID NO: 50 - DNA, 636 bp, Saccharomyces cerevisiae
atggatgctgtaatactgaatctcttaggcgacattcctttggtcacaagattatggacaattggctgtcttgtactatcaggtctcacaagtctccggattgtggatccagggaaggtagtgtacagttatgatttagtattcaaaaagggacaatatggaagactactttattcgatattcgattacggcgcatttaattggatatccatgataaacatctttgtcagcgctaatcacttatcaactttggaaaactcattcaatctgagaagaaaattctgttggataatatttttactgttggtgatactggtaaagatgaccagcattgaacaacctgcagcatcactcggtgtgttattgcatgagaatctcgtgtactacgaactgaaaaagaacggaaaccaaatgaacgtacgattcttcggtgccattgatgtttcaccatctatattcccaatctacatgaatgcagtaatgtattttgtatataagcgtagctggttagaaattgccatgaatttcatgccaggtcacgtaatttactacatggatgatataatagggaagatttatggcatcgatttgtgtaaatctccgtacgactggttccgcaacactgaaacaccctaa

SEQ ID NO: 51 - protein, 551 aa, Saccharomyces cerevisiae
MVPENRRKQLAIFVVVTYLLTFYCVYSATKTSVSFLQVTLKLNEGFNLMVLSIFILLNSTLLWQLLTKLLFGELRLIEHEHIFERLPFTIINTLFMSSLFHERYFFTVAFFGLLLLYLKVFHWILKDRLEALLQSINDSTTMKTLIFSRFSFNLVLLAVVDYQIITRCISSIYTNQKSDIESTSLYLIQVMEFTMLLIDLLNLFLQTCLNFWEFYRSQQSLSNENNHIVHGDPTDENTVESDQSQPVLNDDDDDDDDDRQFTGLEGKFMYEKAIDVFTRFLKTALHLSMLIPFRMPMMLLKDVVWDILALYQSGTSLWKIWRNNKQLDDTLVTVTVEQLQNSANDDNICIICMDELIHSPNQQTWKNKNKKPKRLPCGHILHLSCLKNWMERSQTCPICRLPVFDEKGNVVQTTFTSNSDITTQTTVTDSTGIATDQQGFANEVDLLPTRTTSPDIRIVPTQNIDTLAMRTRSTSTPSPTWYTFPLHKTGDNSVGSSRSAYEFLITNSDEKENGIPVKLTIENHEVNSLHGDGGEQIAKKIVIPDKFIQHI

SEQ ID NO: 52 - DNA, 1656 bp, Saccharomyces cerevisiae
atggtgccagaaaatagaaggaaacagttggcaatttttgtagttgtcacatatttgctcacattttattgcgtgtattcagccaccaagacaagcgtttcctttttgcaagtaacactgaagctaaatgaaggcttcaatctaatggttttgtcgatattcatcttattaaattctaccttactatggcaactcctaacgaaactattatttggtgaactgaggcttattgagcatgagcacatttttgaaaggttaccatttaccattataaacaccttgtttatgtcctcactgttccacgaacggtattttttcacagtggcattttttggactattactactctatctgaaagttttccattggattttaaaggataggctggaggccttattacagtcaataaatgattccaccacaatgaaaacccttatctttagtagattctcatttaacctcgtactattggcggttgtagactaccagataataacacgatgcatctcctccatatatacaaaccaaaagagtgatattgaatccacatccctttacctgatacaagtaatggagtttaccatgcttttgattgatttgctaaatttattcctacagacttgtttgaatttctgggaattttatcgctcacaacaaagtctgtctaatgagaacaaccatattgtccatggcgatcctacagatgaaaacacggttgagtctgatcaatctcagccagtgctgaatgacgacgacgatgacgacgatgatgatagacaatttaccggcctggagggtaaattcatgtatgaaaaagcaattgacgtattcacaagattcttaaaaacggcacttcatttgtctatgctaataccatttaggatgcctatgatgcttttgaaagatgtggtgtgggatatcttggcactatatcaaagtggcacaagtttgtggaaaatctggagaaataacaaacagctcgacgacactcttgtcactgtcaccgtagaacagctacaaaattctgcaaatgatgacaatatttgtatcatttgtatggatgagttaatacattctccaaaccagcagacgtggaagaataaaaacaagaaacccaaaaggttaccttgtggccacatacttcatttgtcgtgtttaaagaattggatggaacgttctcagacttgtcctatttgtagattgcctgtctttgatgaaaaaggtaatgttgtgcaaacgactttcacttccaatagtgatatcacgacacagaccaccgtaacagatagcactgggatagcgacagatcaacaaggtttcgcaaacgaagtagatctacttcccacaagaacaacttcccctgatataaggatagtgcctactcaaaatatagacacattagcaatgagaacaaggtcaacctctacaccatctcctacgtggtatacgttcccattacataaaactggtgataattctgttgggtcaagccgatcagcctacgaatttttgatcacaaattcagatgagaaagaaaatggtattcctgtcaaattaacaatagaaaatcacgaagtaaattctctgcatggagacgggggcgagcaaattgccaagaaaattgtcataccagataaatttatccagcatatctag

SEQ ID NO: 53 - protein, 833 aa, Saccharomyces cerevisiae
MITLLLYLCVICNAIVLIRADSIADPWPEARHLLNTIAKSRDPMKEAAMEPNADEFVGFYVPMDYSPRNEEKNYQSIWQNEITDSQRHIYELLVQSSEQFNNSEATYTLSQIHLWSQYNFPHNMTLAHKYLEKFNDLTHFTNHSAIFDLAVMYATGGCASGNDQTVIPQDSAKALLYYQRAAQLGNLKAKQVLAYKYYSGFNVPRNFHKSLVLYRDIAEQLRKSYSRDEWDIVFPYWESYNVRISDFESGLLGKGLNSVPSSTVRKRTTRPDIGSPFIAQVNGVQMTLQIEPMGRFAFNGNDGNINGDEDDEDASERRIIRIYYAALNDYKGTYSQSRNCERAKNLLELTYKEFQPHVDNLDPLQVFYYVRCLQLLGHMYFTGEGSSKPNIHMAEEILTTSLEISRRAQGPIGRACIDLGLINQYITNNISQAISYYMKAMKTQANNGIVEFQLSKLATSFPEEKIGDPFNLMETAYLNGFIPAIYEFAVMIESGMNSKSSVENTAYLFKTFVDKNEAIMAPKLRTAFAALINDRSEVALWAYSQLAEQGYETAQVSAAYLMYQLPYEFEDPPRTTDQRKTLAISYYTRAFKQGNIDAGVVAGDIYFQMQNYSKAMALYQGAALKYSIQAIWNLGYMHEHGLGVNRDFHLAKRYYDQVSEHDHRFYLASKLSVLKLHLKSWLTWITREKVNYWKPSSPLNPNEDTQHSKTSWYKQLTKILQRMRHKEDSDKAAEDSHKHRTVVQNGANHRGDDQEEASEILGFQMEDLVTMGCILGIFLLSILMSTLAARRGWNVRFNGAQLNANGNRQQEQQQQQQAQGPPGWDFNVQIFAI

SEQ ID NO: 54 - DNA, 2502 bp, Saccharomyces cerevisiae
atgataacactcttattatacctgtgcgtaatatgtaacgcaatagtgttaataagggctgattcgatagcggacccttggcctgaagcgcgacatctactaaataccatagctaagtccagagacccaatgaaagaagctgctatggaacccaatgcagatgaatttgttggattctatgtaccgatggattattccccacgtaatgaggaaaaaaactaccagagcatttggcaaaacgaaatcacagattctcaacgtcatatttatgaattacttgtacaatcaagtgaacaattcaacaactcagaagcaacatatacacttagccagattcacctttggagtcaatataatttcccgcataatatgactttggcacacaaatacttagaaaaattcaatgatctaacccacttcaccaatcattcggccatcttcgacttagctgtgatgtatgccactgggggatgtgcttctggtaatgatcaaaccgtgatccctcaggattctgctaaagcactgctatattaccaaagggctgcccaactagggaatttaaaggctaagcaagtgctagcttataaatactattctggcttcaatgtcccacgaaattttcataaatctttagtattgtacagggacattgctgaacagctgagaaagtcgtactccagggacgaatgggatattgtcttcccctattgggaaagttacaacgtgagaatatcggattttgagagtggcctattaggtaaaggtttgaattccgttccatcttctacagtaaggaaaagaactacgagaccagatattggttcaccctttattgcgcaagttaacggtgtacagatgaccttgcaaatcgaaccgatgggtaggttcgctttcaacggtaacgatggcaacataaatggcgacgaagatgacgaggatgccagtgaaagacgaatcattcggatatattatgcagctttgaatgattataaaggaacatattcacaaagcagaaattgtgagcgcgccaaaaacttgttggaattaacgtacaaggaatttcagcctcatgtcgacaatttggatcctttgcaagtattttactacgtccgttgcttacaattattggggcacatgtatttcaccggcgaaggctcctcgaagcctaatattcatatggccgaagagatcctgaccacgtcgctagaaataagcagaagggcacagggacctataggtagagcgtgcatagatctgggcttaataaatcaatacatcacaaacaatatttctcaagcaatttcgtattatatgaaagctatgaaaacacaagctaacaatggaatcgtagaattccaattatccaaattggccacttcattccctgaagaaaaaatcggcgacccatttaacttaatggaaactgcctacttgaatggattcattccagccatatatgagtttgcagtaatgatcgaatctggaatgaacagtaagagtagtgtggaaaacactgcttacctgttcaaaacattcgttgacaaaaacgaagctattatggcacctaaactgaggacagcatttgccgcattaatcaacgatcgttcagaagtggctttatgggcttattcccaactagccgagcaaggctacgagactgctcaagtctctgccgcctacttaatgtaccagttgccatatgagtttgaggatcctccaagaaccacagatcagagaaaaactttggcaatttcctactatacaagagcgtttaaacagggaaatatagatgctggtgttgtcgcgggagatatctattttcagatgcagaattacagtaaagctatggctctttatcagggtgcagctttgaagtactctatacaggctatctggaacttagggtacatgcatgagcatgggctaggtgtaaacagagatttccatcttgctaaacgttactacgaccaagtttcagaacacgatcatagattttacttggcttccaaattgagtgttttaaaattacacctaaagtcatggttgacttggatcaccagagaaaaagtaaactactggaaaccttcctcgccacttaaccctaacgaagatactcagcactcgaagacttcatggtacaagcaattgacgaagattctacaaagaatgagacataaggaggatagtgacaaagctgcggaagattctcacaaacacagaactgtagtgcagaatggagctaaccataggggtgacgaccaagaggaggcttccgagattttgggcttccaaatggaggatcttgttacgatgggatgtatcttggggatattcctattaagtatattaatgagtacactggcggcccgtagaggctggaatgtccgtttcaatggagcacaattaaatgcaaatggtaaccggcagcaagagcaacaacaacaacaacaagcacaaggtcccccgggctgggacttcaatgttcagatattcgccatatga

SEQ ID NO: 55 - protein, 165 aa, Saccharomyces cerevisiae
MSKTAQKRLLKELQQLIKDSPPGIVAGPKSENNIFIWDCLIQGPPDTPYADGVFNAKLEFPKDYPLSPPKLTFTPSILHPNIYPNGEVCISILHSPGDDPNMYELAEERWSPVQSVEKILLSVMSMLSEPNIESGANIDACILWRDNRPEFERQVKLSILKSLGF

SEQ ID NO: 56 - DNA, 498 bp, Saccharomyces cerevisiae
atgtcgaaaaccgctcagaaacgtctcctcaaggagcttcaacagttaattaaagattctccacctggtatagtggctggtcccaaatcggagaataacatattcatttgggactgcctaattcaagggcctccagatacgccatacgctgatggtgtttttaatgctaagctagagtttcctaaagactatccgttatctccacctaaacttactttcacacccagcatactacatccaaatatttatccaaatggggaagtgtgcatatccattctacactcccctggtgatgatcctaacatgtacgaattagcggaagaaagatggtcgccagtgcaaagtgtagaaaaaattctattaagtgttatgagcatgttgagtgagcccaatatcgaaagtggtgccaacattgatgcttgcatcttgtggagagataatagacctgaatttgagagacaggtaaagttatccattttgaaatcattaggattctga

SEQ ID NO: 57 - protein, 926 aa, Saccharomyces cerevisiae
MEQNIISTIRDECIRHRSKYLTIAQLTAIAEAKINEFIITGKAKDQDLSSLLDKCIDILSIYKKNSKDIKNIISCKNKGAMISSNSVMIIQLNYVYYKVIHIIVTTNIPHLSEFAKIKLHKSTSDEGNGNNNNNEFQLMNIYNTLLETLLKDENIAKIKSFIKSSIKQTKLNHEQEECNLMRTGSYITSNQLNSLISSSANSASSQMEILLIDIRSRLEFNKSHIDTKNIICLEPISFKMSYSDHDLEKKSLITSPNSEIKMFQSRNLFKFIILYTDANEYNVKQQSVLLDILVNHSFEKPISDDFTKIFILESGFPGWLKSNYGRQVSSSFPSNNNIKDDSVYINGNTSGLSLQHLPKMSPSIRHSMDDSMKEMLVAPTPLNHLQQQQQQQSDNDHVLKRSSSFKKLFSNYTSPNPKNSNSNLYSISSLSISSSPSPLPLHSPDPVKGNSLPINYPETPHLWKNSETDFMTNQREQLNHNSFAHIAPINTKAITSPSRTATPKLQRFPQTISMNLNMNSNGHSSATSTIQPSCLSLSNNDSLDHTDVTPTSSHNYDLDFAVGLENLGNSCYMNCIIQCILGTHELTQIFLDDSYAKHININSKLGSKGILAKYFARLVHMMYKEQVDGSKKISISPIKFKLACGSVNSLFKTASQQDCQEFCQFLLDGLHEDLNQCGSNPPLKELSQEAEARREKLSLRIASSIEWERFLTTDFSVIVDLFQGQYASRLKCKVCSHTSTTYQPFTVLSIPIPKKNSRNNITIEDCFREFTKCENLEVDEQWLCPHCEKRQPSTKQLTITRLPRNLIVHLKRFDNLLNKNNDFVIYPFLLDLTPFWANDFDGVFPPGVNDDELPIRGQIPPFKYELYGVACHFGTLYGGHYTAYVKKGLKKGWLYFDDTKYKPVKNKADAINSNAYVLFYHRVYGV

SEQ ID NO: 58 - DNA, 2781 bp, Saccharomyces cerevisiae
atggagcagaatattattagtaccataagggatgagtgtattcgtcaccggtcgaagtaccttacgatagcacaactaaccgctattgcagaggctaaaattaacgaattcatcataactggtaaggcaaaagatcaagatttgagcagtcttctagataaatgcatcgatattttatctatttacaagaagaactcgaaagatatcaaaaatattatatcgtgcaaaaataagggtgcaatgattagttcaaattccgtaatgattattcaattaaattatgtttactacaaggtaattcacattattgtaacaaccaatattcctcatttaagtgaattcgccaagattaaattacataagagcacgagtgatgagggcaacggtaataacaacaataatgaatttcaactcatgaacatttacaacactttgctggaaaccttattaaaagatgaaaacattgcaaaaataaaaagtttcattaagtcttccataaaacaaacaaaattgaaccatgagcaagaagaatgtaacctgatgagaacgggttcctatatcacttccaatcaattaaactccctaataagttcatcagcaaattctgcttcctcccaaatggagatactactgatagatatacgatcaaggttggaattcaacaagtcacatattgatacaaaaaatattatatgcctggagcctatttcttttaaaatgtcatattcagatcatgatttggagaaaaaatcattaattacttctcctaatagtgagattaaaatgtttcaaagtagaaatcttttcaagtttatcattctctatacagacgcaaacgaatacaatgttaaacagcagtctgtcctgttggacattctggtgaatcattcctttgaaaaaccaatatccgatgactttaccaaaattttcattctggaatctggttttccaggttggcttaagtcaaattatgggaggcaagtatcatcatcttttccatcaaataacaatattaaagatgatagtgtttatattaatggtaacacttctggcctaagtttacaacatttacctaagatgtctcccagtataagacattcaatggacgactctatgaaagaaatgctagttgcgcctactccattaaatcatcttcaacaacagcaacaacagcaatcagacaatgatcatgtgctaaaaagatcttcaagtttcaaaaaattattctcaaattatacgtctcctaatccgaagaattcaaattcaaacttatattctatatcttcgttgtccatatctagttcaccatcgcctttacctctacattcgcctgacccagttaagggcaattcattgccaatcaattatccggaaacgccacatctttggaaaaacagtgagacagattttatgacaaatcaaagagaacagttgaatcacaactcttttgctcacatagctcctatcaacacgaaggccatcacttctccatcaagaactgccacaccgaagttacaacgcttcccgcaaacaattagtatgaaccttaatatgaactccaatggacacagttctgccacctctaccattcaaccttcgtgtctatccttgtctaataatgactctttagatcatacagatgttacaccaacttcttctcataattatgaccttgatttcgcggttggtttggaaaatctaggaaattcgtgttacatgaactgtatcattcagtgtatcttaggtacacacgaattaacccaaatctttttggacgattcatatgctaaacacatcaatattaatagtaagttgggatcgaaaggtattctggcaaaatattttgcaaggttggttcatatgatgtataaggaacaggttgatggttcaaagaaaatttccatatcaccgataaaatttaaattggcatgtggatctgttaactcattatttaagactgcatcccaacaggactgccaagagttttgccaattccttctagatggtcttcatgaagacttgaaccaatgcggttcaaacccacctttgaaggagctttctcaagaagctgaggcgagaagagaaaaactgtctttgcgaattgcctcgtcaattgagtgggaacgattcttgactactgatttcagtgttattgtcgacttatttcagggacaatacgcctcacgactaaaatgtaaagtctgtagtcatacctcgacaacataccaaccttttacggtgctgtcaatccctattcctaaaaaaaattcccgaaataatattaccattgaagattgtttcagagagttcaccaaatgtgagaacttggaagtggatgagcaatggttgtgcccacattgtgaaaaaaggcagccctccacgaaacaattgacaataacgagattaccgaggaatctgatagtccatttaaagagatttgataatttattaaacaaaaataatgacttcgtcatatacccttttttgttggacttgactccattttgggccaatgattttgacggggtttttcctccaggtgttaatgacgatgaactaccaataaggggacaaataccaccttttaagtatgaattatatggtgtagcatgccactttggtactttgtatggtggtcattatacagcctatgtgaaaaagggattaaagaagggatggctatattttgatgataccaaatataaacctgtcaaaaacaaagccgatgcaattaactctaatgcatacgttttgttttatcaccgcgtctacggtgtttga

SEQ ID NO: 59 - protein, 238 aa, Saccharomyces cerevisiae
MEMTDFELTSNSQSNLAIPTNFKSTLPPRKRAKTKEEKEQRRIERILRNRRAAHQSREKKRLHLQYLERKCSLLENLLNSVNLEKLADHEDALTCSHDAFVASLDEYRDFQSTRGASLDTRASSHSSSDTFTPSPLNCTMEPATLSPKSMRDSASDQETSWELQMFKTENVPESTTLPAVDNNNLFDAVASPLADPLCDDIAGNSLPFDNSIDLDNWRNPEAQSGLNSFELNDFFITS

SEQ ID NO: 60 - DNA, 717 bp, Saccharomyces cerevisiae
atggaaatgactgattttgaactaactagtaattcgcaatcgaacttggctatccctaccaacttcaagtcgactctgcctccaaggaaaagagccaagacaaaagaggaaaaggaacagcgaaggatcgagcgtattttgagaaacagaagagctgctcaccagagcagagagaaaaaaagactacatctgcagtatctcgagagaaaatgttctcttttggaaaatttactgaacagcgtcaaccttgaaaaactggctgaccacgaagacgcgttgacttgcagccacgacgcttttgttgcttctcttgacgagtacagggatttccagagcacgaggggcgcttcactggacaccagggccagttcgcactcgtcgtctgatacgttcacaccttcacctctgaactgtacaatggagcctgcgactttgtcgcccaagagtatgcgcgattccgcgtcggaccaagagacttcatgggagctgcagatgtttaagacggaaaatgtaccagagtcgacgacgctacctgccgtagacaacaacaatttgtttgatgcggtggcctcgccgttggcagacccactctgcgacgatatagcgggaaacagtctaccctttgacaattcaattgatcttgacaattggcgtaatccagaagcgcagtcaggtttgaattcatttgaattgaatgatttcttcatcacttcatga

SEQ ID NO: 61 - protein, 663 aa, Saccharomyces cerevisiae
MPTNYEYDEASETWPSFILTGLLMVVGPMTLLQIYQIFFGANAEDGNSGKSKEFNEEVFKNLNEEYTSDEIKQFRRKFDKNSNKKSKIWSRRNIIIIVGWILVAILLQRINSNDAIKDAATKLFDPYEILGISTSASDRDIKSAYRKLSVKFHPDKLAKGLTPDEKSVMEETYVQITKAYESLTDELVRQNYLKYGHPDGPQSTSHGIALPRFLVDGSASPLLVVCYVALLGLILPYFVSRWWARTQSYTKKGIHNVTASNFVSNLVNYKPSEIVTTDLILHWLSFAHEFKQFFPDLQPTDFEKLLQDHINRRDSGKLNNAKFRIVAKCHSLLHGLLDIACGFRNLDIALGAINTFKCIVQAVPLTPNCQILQLPNVDKEHFITKTGDIHTLGKLFTLEDAKIGEVLGIKDQAKLNETLRVASHIPNLKIIKADFLVPGENQVTPSSTPYISLKVLVRSAKQPLIPTSLIPEENLTEPQDFESQRDPFAMMSKQPLVPYSFAPFFPTKRRGSWCCLVSSQKDGKILQTPIIIEKLSYKNLNDDKDFFDKRIKMDLTKHEKFDINDWEIGTIKIPLGQPAPETVGDFFFRVIVKSTDYFTTDLDITMNMKVRDSPAVEQVEVYSEEDDEYSTDDDETESDDESDASDYTDIDTDTEAEDDESPE

SEQ ID NO: 62 - DNA, 1992 bp, Saccharomyces cerevisiae
atgcctacaaattacgagtatgatgaggctagtgagacgtggccgtccttcattttaacggggctcttgatggtcgtcgggcctatgacactgcttcaaatataccaaattttttttggggccaatgctgaagatgggaattcagggaagagtaaggagtttaatgaggaagttttcaagaacttgaatgaagaatacaccagtgatgaaatcaaacaatttagaaggaagtttgataaaaatagtaataagaagtccaaaatatggagcaggagaaatattataattattgtgggttggatcttagttgcaattcttctgcaaaggattaatagtaatgacgcgattaaagacgctgctacaaaattatttgatccttatgaaatccttggtatctctactagtgcttccgatagagacatcaaatctgcttatagaaaattatctgttaaatttcatccagataaattagcaaagggcctaacacctgatgagaaaagtgtgatggaagaaacttatgttcagattacgaaggcttacgaatcccttactgacgaattggttaggcaaaactatttgaaatacggtcatccagatggcccacaatctacttcacatggtatcgctctaccaagatttttggtagatggaagtgcatctccattattagtggtttgttatgttgcgctactaggtttaatcttgccatattttgttagtagatggtgggcaagaacacaatcgtatactaagaagggaatacataatgtgacggcttctaattttgttagtaacttagtcaattacaagccatctgagattgtcaccacagatttgatcttacactggttatcatttgctcatgaatttaaacaattcttcccggatttgcaaccaacggattttgaaaaacttttgcaagatcatattaaccgcagagatagtggtaaacttaacaatgcgaaatttagaatagtggccaaatgtcactctttgttacacggtttattggatattgcttgtggattcagaaatttagatattgcattgggtgcaatcaatactttcaagtgtattgttcaggctgtaccattaacaccaaactgtcaaatccttcaattgccgaacgtagataaagagcactttattaccaaaaccggagatattcatacattaggtaaattgtttactttagaagatgccaagattggtgaggttcttggaataaaggatcaagcaaagttaaacgaaactttgagagttgcatcgcatattccaaatctaaagatcatcaaggcagacttccttgtcccaggtgagaaccaagtaacaccatcatctaccccatacatttctttgaaagtactggttcgttctgctaaacagccattgataccaactagcttaattcctgaagaaaatttaacagaacctcaagattttgaatctcaaagagatccatttgctatgatgagtaaacagccactcgtcccatattcctttgcaccatttttccctacaaagagacgtgggagttggtgctgtctggtaagttctcaaaaagatggtaaaatacttcaaacgccaattatcattgaaaagctatcttacaagaacttgaacgatgacaaagatttctttgataagaggataaaaatggatttaaccaaacacgaaaaattcgatataaatgattgggaaatcgggaccataaaaattccattaggtcagcctgcacctgaaactgttggtgatttcttttttagagtaatcgttaaatccacagattatttcactacagatttggatattaccatgaatatgaaagttcgtgattctcctgcagtggaacaagtagaggtgtattctgaggaggatgatgagtactctactgatgacgacgaaaccgaaagtgatgatgaaagtgatgctagcgattatactgatatcgatacggatacagaagctgaagatgatgaatcaccagaatag

SEQ ID NO: 63 - protein, 409 aa, Saccharomyces cerevisiae
MVKETKFYDILGVPVTATDVEIKKAYRKCALKYHPDKNPSEEAAEKFKEASAAYEILSDPEKRDIYDQFGEDGLSGAGGAGGFPGGGFGFGDDIFSQFFGAGGAQRPRGPQRGKDIKHEISASLEELYKGRTAKLALNKQILCKECEGRGGKKGAVKKCTSCNGQGIKFVTRQMGPMIQRFQTECDVCHGTGDIIDPKDRCKSCNGKKVENERKILEVHVEPGMKDGQRIVFKGEADQAPDVIPGDVVFIVSERPHKSFKRDGDDLVYEAEIDLLTAIAGGEFALEHVSGDWLKVGIVPGEVIAPGMRKVIEGKGMPIPKYGGYGNLIIKFTIKFPENHFTSEENLKKLEEILPPRIVPAIPKKATVDECVLADFDPAKYNRTRASRGGANYDSDEEEQGGEGVQCASQ

SEQ ID NO: 64 - DNA, 1230 bp, Saccharomyces cerevisiae
atggttaaagaaactaagttttacgatattctaggtgttccagtaactgccactgatgtcgaaattaagaaagcttatagaaaatgcgccttaaaataccatccagataagaatccaagtgaggaagctgcagaaaagttcaaagaagcttcagcagcctatgaaattttatcagatcctgaaaagagagatatatatgaccaatttggtgaagatggtctaagtggtgctggtggcgctggcggattcccaggtggtggattcggttttggtgacgatatcttttcccaattctttggtgctggtggcgcacaaagaccaagaggtccccaaagaggtaaagatatcaagcatgaaatttctgcctcacttgaagaattatataagggtaggacagctaagttagcccttaacaaacagatcctatgtaaagaatgtgaaggtcgtggtggtaagaaaggcgccgtcaagaagtgtaccagctgtaatggtcaaggtattaaatttgtaacaagacaaatgggtccaatgatccaaagattccaaacagagtgtgatgtctgtcacggtactggtgatatcattgatcctaaggatcgttgtaaatcttgtaacggtaagaaagttgaaaacgaaaggaagatcctagaagtccatgtcgaaccaggtatgaaagatggtcaaagaatcgttttcaaaggtgaagctgaccaagccccagatgtcattccaggtgatgttgtcttcatagtttctgagagaccacacaagagcttcaagagagatggtgatgatttagtatatgaggctgaaattgatctattgactgctatcgctggtggtgaatttgcattggaacatgtttctggtgattggttaaaggtcggtattgttccaggtgaagttattgccccaggtatgcgtaaggtcatcgaaggtaaaggtatgccaattccaaaatacggtggctatggtaatttaatcatcaaatttactatcaagttcccagaaaaccatttcacatcagaagaaaacttgaagaagttagaagaaattttgcctccaagaattgtcccagccattccaaagaaagctactgtggacgaatgtgtactcgcagactttgacccagccaaatacaacagaacacgggcctccaggggtggtgcaaactatgattccgatgaagaagaacaaggtggcgaaggtgttcaatgtgcatctcaatga

SEQ ID NO: 65 - protein, 459 aa, Saccharomyces cerevisiae
MSGSDRGDRLYDVLGVTRDATVQEIKTAYRKLALKHHPDKYVDQDSKEVNEIKFKEITAAYEILSDPEKKSHYDLYGDDNGAASSGGANGFGDEDFMNFFNNFFNNGSHDGNNFPGEYDAYEEGNSTSSKDIDIDISLTLKDLYMGKKLKFDLKRQVICIKCHGSGWKPKRKIHVTHDVECESCAGKGSKERLKRFGPGLVASQWVVCEKCNGKGKYTKRPKNPKNFCPDCAGLGLLSKKEIITVNVAPGHHFNDVITVKGMADEEIDKTTCGDLKFHLTEKQENLEQKQIFLKNFDDGAGEDLYTSITISLSEALTGFEKFLTKTFDDRLLTLSVKPGRVVRPGDTIKIANEGWPILDNPHGRCGDLYVFVHIEFPPDNWFNEKSELLAIKTNLPSSSSCASHATVNTEDDSNLTNNETISNFRIIHTDDLPEGIRPFKPEAQDSAHQKARSSYCCIQ

SEQ ID NO: 66 - DNA, 1380 bp, Saccharomyces cerevisiae
atgagtggcagtgatagaggagaccggttatacgatgtgttgggggtgacgagagatgcgaccgtgcaagagattaaaactgcttacagaaagcttgccctgaaacatcatccggacaagtatgtggatcaagactcaaaggaggtaaatgaaatcaaattcaaagagatcactgccgcttacgagatcttgagcgatccggagaagaaatcacattacgacttgtatggtgatgataatggtgccgctagcagcggtggcgctaatggctttggagatgaagattttatgaacttctttaacaatttcttcaataatggaagtcacgatggaaataatttccctggcgagtatgatgcgtacgaagagggcaactctacaagctctaaggatatcgatatcgatatatctcttactttgaaggatttgtacatgggcaagaagctgaagtttgatttaaagagacaggtcatctgtataaagtgccacggttctggctggaaaccaaagaggaaaattcacgttacacacgatgtggaatgtgaatcatgcgctggaaagggttcaaaggaacgtctgaagaggtttggtcccggtttggtagcttcgcaatgggtggtctgtgagaaatgtaatggtaaggggaagtacactaaaagacccaagaatccaaagaacttttgccccgattgcgcaggcttggggctcctgtcaaagaaggaaatcatcacagtgaacgtggctccgggacaccactttaacgacgtaattacagtcaaggggatggcggacgaggaaatcgataagaccacatgtggtgatttaaagttccatctcactgaaaaacaagaaaacttggagcagaagcaaatctttttgaagaactttgacgacggcgccggggaagatttgtatacaagcattaccatatcgttaagcgaggccttgacgggatttgagaaatttttgacaaaaaccttcgacgacaggttactaacattgagcgttaaacctggcagagtagtaagacctggtgacaccatcaaaatcgccaatgaaggttggcccattttagataaccctcatggccggtgcggcgatctgtatgttttcgttcatattgaatttccaccagataactggttcaatgaaaaatcagaactactagcaataaaaacgaatctgccgtcatcttcatcttgtgcctcacatgcgactgtaaatactgaagatgacagcaacctgactaacaacgaaactatatcaaatttccggatcattcacacggacgatcttccagaagggataaggccgttcaagccagaagcacaggattcagcgcatcagaaagcaagaagttcgtactgctgtatccaatga

SEQ ID NO: 67 - protein, 528 aa, Saccharomyces cerevisiae
MQQNTSLYDSLNVTAAASTSEIKKAYRNAALKYHPDKNNHTEESKRKFQEICQAYEILKDNRLRALYDQYGTTDEVLIQEQQAQAQRQQAGPFSSSSNFDTEAMSFPDLSPGDLFAQFFNSSATPSSNGSKSSFNFSFNNSSTPSFSFVNGSGVNNLYSSSAKYNSNDEDHHLDRGPDIKHNLKCTLKELYMGKTAKLGLNRTRICSVCDGHGGLKKCTCKTCKGQGIQTQTRRMGPLVQSWSQTCADCGGAGVFVKNKDICQQCQGLGFIKERKILQVTVQPGSCHNQLIVLTGEGDEVISTKGGGHEKVIPGDVVITILRLKDPNFQVINYSNLICKKCKIDFMTSLCGGVVYIEGHPSGKLIKLDIIPGEILKPGCFKTVEDMGMPKFINGVRSGFGHLYVKFDVTYPERLEPENAKKIQNILANDKYIKAERSTMETADSDCYCDLEKSYDSVEEHVLSSFEAPNLNNEVIEDDDLGDLINERDSRKRNNRRFDESNINNNNETKRNKYSSPVSGFYDHDINGY

SEQ ID NO: 68 - DNA, 1587 bp, Saccharomyces cerevisiae
atgcaacaaaacacgtctttatatgactctttgaacgttactgccgctgcatccacatctgagattaagaaagcttacaggaacgctgcattaaaatatcatcctgataaaaacaatcatacagaagaatccaagcgaaagtttcaagagatatgccaggcatacgaaatacttaaagacaatcgtttaagagctttgtatgaccagtacggtaccacagatgaagtcctgattcaagagcagcaggcgcaggcgcaacgccaacaagccgggccgttcagttcatcctcaaatttcgatacggaagcaatgtcattcccggatctatctccaggtgatcttttcgcgcagttttttaatagttctgctaccccctcttctaatggctccaaaagcagttttaattttagcttcaataatagctctacgccgagcttctcctttgttaatggcagtggcgtgaacaatctgtactcctcgtcagcaaaatacaactccaacgatgaggaccatcatttggatagaggccctgatatcaaacataatctaaagtgcacattgaaggaactctacatgggtaagactgcaaagttgggtttgaataggacaaggatttgcagtgtttgtgatgggcacggtggtctaaagaaatgcacttgtaaaacatgcaaagggcaaggtattcaaacccaaactaggcgtatgggacctctagtacaaagttggtctcaaacttgtgcagattgcgggggtgccggggtttttgtcaaaaataaagatatttgccaacagtgccaaggtcttggcttcattaaggagaggaagattctacaagtcaccgttcaaccgggatcgtgtcataaccaacttatagtacttacgggcgaaggtgacgaagttattagtactaagggaggcggtcacgaaaaggtaatacctggtgacgtcgttatcaccattttacgtttaaaagatccgaatttccaggttatcaactactccaatttgatatgtaagaagtgcaaaatcgacttcatgaccagtttatgtggaggcgtagtttatattgaagggcaccctagcggtaagttgatcaaacttgatattatacctggcgagatactgaagcctggttgtttcaagactgttgaggacatggggatgcccaagtttatcaacggtgttcggagcggtttcggtcatctatatgtcaaattcgatgtgacgtatccagagagactggaacctgaaaatgctaagaaaatacaaaatattctggctaatgataaatacattaaagcagaacgttccaccatggaaaccgcagattcagactgctattgcgatttggagaagtcatatgacagtgtggaagagcatgtgttaagtagctttgaggcccctaatttaaacaatgaagttattgaagacgacgaccttggtgatttgattaatgaaagagattctcggaaaaggaacaaccgtcgattcgacgaaagtaatattaataataataatgaaacgaaacgaaataaatattcttcaccggtaagcggtttttatgaccatgatattaatggatattga

SEQ ID NO: 69 - protein, 352 aa, Saccharomyces cerevisiae
MVKETKLYDLLGVSPSANEQELKKGYRKAALKYHPDKPTGDTEKFKEISEAFEILNDPQKREIYDQYGLEAARSGGPSFGPGGPGGAGGAGGFPGGAGGFSGGHAFSNEDAFNIFSQFFGGSSPFGGADDSGFSFSSYPSGGGAGMGGMPGGMGGMHGGMGGMPGGFRSASSSPTYPEEETVQVNLPVSLEDLFVGKKKSFKIGRKGPHGASEKTQIDIQLKPGWKAGTKITYKNQGDYNPQTGRRKTLQFVIQEKSHPNFKRDGDDLIYTLPLSFKESLLGFSKTIQTIDGRTLPLSRVQPVQPSQTSTYPGQGMPTPKNPSQRGNLIVKYKVDYPISLNDAQKRAIDENF

SEQ ID NO: 70 - DNA, 1059 bp, Saccharomyces cerevisiae
atggtcaaggagacaaaactttatgatttacttggagtatctccaagtgctaatgagcaagaactgaaaaagggttatagaaaagcagctctaaaatatcatccagataagccaacaggtgacacagaaaagtttaaggagatatcagaggcctttgaaattttaaatgatcctcaaaaaagggaaatatatgatcaatacggtctcgaggctgctagatctggtggtccaagctttggtcctggtggtcctggcggtgctggaggtgctggaggcttccctggcggtgcgggcggattctccggaggacatgcgttcagtaatgaggatgctttcaatattttttcacaattctttggcggcagttccccattcggtggtgctgatgacagtggcttcagtttctctagttatccatctggcggcggtgctggtatgggaggtatgcctggaggaatgggaggaatgcatggcggcatgggaggtatgcctggcggctttagatcagcatcaagctctcccacgtatccagaggaagaaacagttcaagttaatttaccagttagtctagaagatttgtttgttggtaaaaagaagtcatttaaaattggaagaaagggcccacatggggcctctgaaaagacacaaattgacattcaattaaaaccgggttggaaagctggtaccaaaataacatacaagaaccagggtgattacaatcctcaaacgggccgtagaaagactttgcagtttgtcatccaggaaaagagccatccaaactttaaaagagacggtgatgacctaatttacactctgccactatctttcaaggaatcattgttaggtttttcaaaaactatccaaacaattgatggcagaaccttacctttgtcgagagtacagcctgtccaaccctcacaaacttctacttatcctggtcaaggtatgccaactccaaagaacccatctcagagaggtaatttgattgtaaaatataaagtggactatccaatatcactaaacgacgctcaaaaacgtgctatagatgaaaatttttaa

SEQ ID NO: 71 - protein, 432 aa, Saccharomyces cerevisiae
MVVDTEYYDLLGVSTTASSIEIKKAYRKKSIQEHPDKNPNDPTATERFQAISEAYQVLGDDDLRAKYDKYGRKEAIPQGGFEDAAEQFSVIFGGDAFASYIGELMLLKNLQKTEELNAEDEAEKEKENVETMEESPADGKTNGTTNAVDAALGNTNEKDDKNKARTTSGNLTVHDGNKKNEQVGAEAKKKKTKLEQFEEEQEVEKQKRVDQLSKTLIERLSILTESVYDDACKDSFKKKFEEEANLLKMESFGLDILHTIGDVYYEKAEIFLASQNLFGMGGIFHSMKAKGGVFMDTLRTVSAAIDAQNTMKELEKMKEASTNNEPLFDKDGNEQIKPTTEELAQQEQLLMGKVLSAAWHGSKYEITSTLRGVCKKVLEDDSVSKKTLIRRAEAMKLLGEVFKKTFRTKVEQEEAQIFEELVAEATKKKRHT

SEQ ID NO: 72 - DNA, 1299 bp, Saccharomyces cerevisiae
atggttgttgatactgagtattacgatttgttaggtgtgtctaccactgcatcttccattgaaataaaaaaggcctatagaaagaaatctattcaagagcatcctgataagaatcccaatgaccccacggctaccgaaaggtttcaagcaatatccgaagcttatcaagttttaggtgacgatgatcttcgcgcaaagtatgataagtatggaagaaaagaagctattcctcagggcggctttgaagatgcagctgaacagttctctgtcatctttggtggagatgcgtttgcctcatatattggcgaactgatgctattaaagaacctacagaaaactgaggagctaaatgctgaagacgaagctgaaaaggagaaggagaatgtggaaacaatggaagaatcacctgcagacggtaagacgaatggcaccactaacgctgttgatgcagcattgggcaatactaacgaaaaagatgacaaaaataaggcgaggacaacttctggtaatttaactgtacacgatggaaacaagaaaaatgagcaggtaggagcagaagctaagaagaagaagacaaaattagagcagtttgaggaagaacaagaggtagaaaagcaaaaaagagtagaccaattaagcaaaacattgattgaaagattatcgatattaacagaaagtgtctatgatgatgcatgtaaagattcctttaaaaaaaagttcgaagaggaagccaatcttttaaagatggaatcatttggtctggacatattacacacaataggcgacgtttactacgaaaaagctgaaatttttcttgcatcccagaacctgttcggaatgggtggtatatttcattctatgaaggctaaagggggagtatttatggatacactaagaactgtttcggcagccatagacgctcagaatactatgaaggagcttgaaaaaatgaaagaagctagcacgaataatgagcctttgtttgacaaagacggaaatgagcaaattaagccaaccactgaggaactggcgcagcaagagcagctattgatgggcaaagtattgtcggctgcttggcatggttctaaatatgaaataacatccactttacgtggcgtttgtaaaaaagtactagaagatgactcggtaagtaagaaaacgcttatcagaagagctgaagcaatgaaactattgggtgaagtctttaagaaaactttcagaaccaaagtcgaacaagaagaggcacagatctttgaagaacttgtagcagaagctacaaaaaagaagagacatacatga

SEQ ID NO: 73 - protein, 433 aa, Saccharomyces cerevisiae
MFSLPTLTSDITVEVNSSATKTPFVRRPVEPVGKFFLQHAQRTLRNHTWSEFERIEAEKNVKTVDESNVDPDELLFDTELADEDLLTHDARDWKTADLYAAMGLSKLRFRATESQIIKAHRKQVVKYHPDKQSAAGGSLDQDGFFKIIQKAFETLTDSNKRAQYDSCDFVADVPPPKKGTDYDFYEAWGPVFEAEARFSKKTPIPSLGNKDSSKKEVEQFYAFWHRFDSWRTFEFLDEDVPDDSSNRDHKRYIERKNKAARDKKKTADNARLVKLVERAVSEDPRIKMFKEEEKKEKERRKWEREAGARAEAEAKAKAEAEAKAKAESEAKANASAKADKKKAKEAAKAAKKKNKRAIRNSAKEADYFGDADKATTIDEQVGLIVDSLNDEELVSTADKIKANAAGAKEVLKESAKTIVDSGKLPSSLLSYFV

SEQ ID NO: 74 - DNA, 1302 bp, Saccharomyces cerevisiae
atgttttctttacctaccctaacctcagacatcactgttgaagtcaacagttccgctaccaaaaccccattcgtccgtcgtccggtcgaaccggttggtaagttctttttgcaacatgctcaaagaactttgagaaaccacacctggtctgaatttgaaagaattgaagctgaaaagaacgtcaaaaccgttgatgaatccaatgtcgacccagatgagttgttattcgacactgaattggccgatgaagatttactgactcatgatgctagagactggaaaactgccgatttgtatgctgctatgggtttgtctaagttgcgtttcagagctactgaaagtcaaatcatcaaggctcacagaaaacaagttgtcaagtaccatccagacaagcaatctgctgctggtggtagtttggaccaagatggctttttcaagattattcaaaaggcctttgaaactttgactgattccaacaagagagctcagtacgactcatgtgattttgttgccgatgttcctcctccaaagaagggtaccgattatgacttttatgaagcttggggccccgttttcgaagctgaagctcgtttttctaagaagactcctattccttctctaggtaacaaagattcttccaagaaggaagttgaacaattctatgctttctggcacagatttgactcctggagaacctttgagttcttggacgaagatgtcccagatgactcttctaacagagaccacaagcgttacattgaaagaaagaacaaggccgcaagagacaagaagaagactgctgataacgctagattggtcaaacttgttgaaagagctgtcagtgaagatccccgtatcaaaatgttcaaagaagaagagaagaaggaaaaggaaagaagaaaatgggaaagagaagccggtgccagagctgaagctgaagctaaggccaaggccgaagctgaagcgaaggctaaagctgaatctgaagccaaggctaacgcctccgcaaaagctgacaaaaagaaggctaaggaagctgctaaggccgccaagaaaaagaacaagagagccatccgtaactctgctaaggaagctgactactttggtgatgctgacaaggccaccacgattgacgaacaagttggtttgatcgttgacagtttgaatgacgaagagttagtgtccaccgccgataagatcaaggccaatgctgctggtgccaaggaagttttgaaggaatctgcaaagactattgtcgattctggcaaactaccatccagcttgttgtcctacttcgtgtga

SEQ ID NO: 75 - protein, 668 aa, Saccharomyces cerevisiae
MSDPFAHLLTSLKNKDSASASKETTPQSSNSPSITGSAVADVARTDKSPNDSLHSISAPPLIPSPKVDFSAPPLVPTNSTTKSNTANNTPPSALANTDDDFNQLFGMGTVTTTDTIQKPDEDYYGSKEDHLYNGDDALVDEVKDMEIARLMSLGLSIEEATEFYENDVTYERYLEILKSKQKERNDLAIRKKESGIKMEKSGLSNIVGTDSNNLFSMATDFFNKGKKLVDQWTSFPPEANDRLNNYSKTHDKVEDYDLPQVNDSPNRILFEDNEVVENLPPADNPDQDLLTDFETKIDITKRTAPDVSHSSSPTSGILIEENSRRNEPLIEDSLLDFSEGNLTNSKSNEDSTLFNENSNTDSTIPISDIELSGYNEFKAKGTSLFKNGDYINSLQEYEKSLNTLPLNHPLRIIALSNIIASQLKIGEYSKSIENSSMALELFPSSKAKWKNKISNSDPERSFNDIWPKIMIRRAESFEHLESFKKALETYQELIKKNFFDDKIMQGKRRCQDFINPPPVKKSMPVKKKTTTTSPATKKQNLTASSSNSPISVDSTSEIKKRELENAKLALYDKVFEKISSWKDGKDDDIRHLLANLSSLLTWCNWKDVSMQDLVMPKRVKITYMKAVAKTHPDKIPESLSLENKMIAENIFSTLSIAWDKFKLQNDIN

SEQ ID NO: 76 - DNA, 2007 bp, Saccharomyces cerevisiae
atgtcagatccatttgcacatttactgacttctttgaagaataaggactctgcatctgcatccaaggaaacaactcctcagagcagcaattcgccttccattactggttccgctgttgcagatgttgcaaggacggataaaagccccaatgatagtctgcattcaatttcagctcctccgctgataccgtcaccgaaggtagatttttctgcacctcctttggtcccaactaatagcaccactaaatctaatactgccaacaacacacctccctcggctcttgccaataccgatgacgacttcaatcaactatttggtatgggcacagtaactacaacggatacgatccaaaaaccggatgaggattactatggaagcaaggaagaccacctttacaatggtgatgacgccttagttgatgaagttaaggatatggaaatagcaagattgatgtctctaggtttatcaattgaagaagccactgagttttacgaaaatgacgtaacttatgaaagatatttggagattttaaagtcaaagcaaaaggagcgcaacgatctagctataagaaagaaagaaagtggtataaaaatggaaaagtcaggattatccaacattgttggtacagatagcaataatttattcagcatggccactgattttttcaataagggtaagaaactggtagaccaatggacctccttcccacctgaggcaaatgatagactgaataattactcaaaaactcatgataaggttgaggattatgatttgcctcaagtaaacgactcacccaatagaattttgtttgaagataatgaagtcgtagagaacttaccacctgccgataatccggatcaagatcttttaactgatttcgaaacaaagattgatataacaaagaggacagcgcctgatgtctcccactcctcctcaccgacttctggtatactaattgaagaaaattcgcgaagaaatgagcccctgatagaggatagtcttctcgacttttcagaaggaaatctcaccaatagtaaaagcaatgaagatagcaccctcttcaatgaaaacagcaacactgactctacaatacccatctcagatattgaattatcggggtataacgaatttaaggcgaaaggtactagtttgttcaagaacggggattatattaactcattacaagaatatgaaaagtctttaaatacattgcctttaaatcatccattgaggatcattgcattatcaaacattattgcctcgcaactgaaaatcggtgagtactctaagtccatagaaaactccagcatggctttggaattattcccatcaagcaaagctaagtggaagaataaaatctcaaatagtgaccctgaaagatcatttaacgacatctggccaaagattatgattaggcgtgctgagtcttttgaacatttagaaagtttcaaaaaagcactagaaacataccaagagctgattaagaagaatttttttgatgataaaatcatgcagggaaaaagaagatgccaagactttattaatcctccccctgttaaaaaatccatgcccgttaagaagaagacaacgacaacctcgcctgcaacaaaaaaacagaacttaaccgcttcttcttcaaattctccaatttctgttgatagcacttcagaaataaaaaaacgggagctagaaaacgctaaactggcgctatatgataaagtatttgagaaaattagctcctggaaggatggcaaagacgatgacattcgtcatctgttagcaaatttatccagcttactaacatggtgcaattggaaggatgtctctatgcaagatttggttatgcctaagagggtcaaaattacatacatgaaagctgtagccaagacacatcctgataagataccagagtccttgtccctggaaaataagatgattgcagagaatattttcagtactttaagtattgcttgggataagttcaaactgcagaatgacattaactga

SEQ ID NO: 77 - protein, 590 aa, Saccharomyces cerevisiae
MKTCYYELLGVETHASDLELKKAYRKKALQYHPDKNPDNVEEATQKFAVIRAAYEVLSDPQERAWYDSHKEQILNDTPPSTDDYYDYEVDATVTGVTTDELLLFFNSALYTKIDNSAAGIYQIAGKIFAKLAKDEILSGKRLGKFSEYQDDVFEQDINSIGYLKACDNFINKTDKLLYPLFGYSPTDYEYLKHFYKTWSAFNTLKSFSWKDEYMYSKNYDRRTKREVNRRNEKARQQARNEYNKTVKRFVVFIKKLDKRMKEGAKIAEEQRKLKEQQRKNELNNRRKFGNDNNDEEKFHLQSWQTVKEENWDELEKVYDNFGEFENSKNDKEGEVLIYECFICNKTFKSEKQLKNHINTKLHKKNMEEIRKEMEEENITLGLDNLSDLEKFDSADESVKEKEDIDLQALQAELAEIERKLAESSSEDESEDDNLNIEMDIEVEDVSSDENVHVNTKNKKKRKKKKKAKVDTETEESESFDDTKDKRSNELDDLLASLGDKGLQTDDDEDWSTKAKKKKGKQPKKNSKSTKSTPSLSTLPSSMSPTSAIEVCTTCGESFDSRNKLFNHVKIAGHAAVKNVVKRKKVKTKRI

SEQ ID NO: 78 - DNA, 1773 bp, Saccharomyces cerevisiae
atgaagacctgctactatgagcttttaggggtcgaaacgcatgcttctgatcttgagttaaaaaaagcttaccgtaaaaaggccctacaatatcacccagataaaaacccagataatgttgaagaagccacacaaaaatttgctgtgattcgagccgcttatgaagtactgtctgacccccaggaaagagcatggtatgactcacataaggaacaaattttaaatgatactccaccaagcactgatgattactatgattatgaggtagacgctacagtcacaggtgtcacaactgatgaattactcttattttttaactctgctctttatactaaaatagacaactcagctgctgggatatatcaaattgcaggaaaaatatttgccaagttagctaaagatgagattttaagtggtaagcgactggggaaattttccgagtatcaagatgatgtattcgaacaggatattaatagtattggctatttgaaagcctgcgataactttattaacaagacggataaacttttatatcctttatttggatattcgccaacggattatgaatatttgaaacatttctataagacttggtcagcgttcaataccttgaaaagttttagctggaaagacgagtacatgtactctaaaaactatgacagaagaaccaagagggaagttaatagaagaaatgagaaggctaggcaacaagctcgaaatgaatacaacaaaaccgtgaaaaggtttgtagttttcataaaaaagctcgataaaagaatgaaagaaggtgcaaaaattgcagaagaacagcgtaaactaaaagaacaacagaggaaaaatgagttaaataacagaagaaagtttgggaacgacaacaatgacgaagaaaaatttcatttacaaagctggcaaacggtaaaagaagaaaactgggatgaactggaaaaggtatatgataattttggagaattcgaaaattctaagaatgataaggaaggtgaagtattgatttacgagtgttttatctgcaacaagacatttaagtcggaaaagcaattgaaaaaccacataaacactaaactgcataagaaaaatatggaagagatacggaaagaaatggaagaggaaaacataacgcttgggttggataatctctccgatctcgagaaatttgattcagcagatgaaagtgttaaagaaaaagaagatattgatctgcaagcattgcaagctgaactcgctgaaattgaaagaaaactggcagaatcgtcttctgaagacgaaagtgaagatgacaatctcaacatagaaatggatatagaggtagaagacgtcagttcggatgaaaatgtacatgtgaatacgaagaataaaaagaaaagaaaaaagaaaaaaaaagcaaaggttgacacagaaacagaggaatctgaatcgttcgatgatactaaagacaaacggagtaatgagttggatgatcttttggcatcactaggagacaagggcttacaaacggatgacgatgaagattggtctactaaagcgaaaaagaaaaagggcaaacaacctaaaaagaattctaaatccacaaaaagcactccgtccttgtcgactctaccgtcctctatgtctccaacctccgcgatcgaggtgtgcactacatgcggagaatcatttgatagtcgaaataagctattcaaccacgtgaagatagcagggcatgcggcagtgaaaaacgtagtgaaaagaaagaaagtcaagaccaaaagaatatag

SEQ ID NO: 79 - protein, 583 aa, Saccharomyces cerevisiae
MSQVIEPQLDRTTYYSILGLTSNATSSEVHKSYLKLARLLHPDKTKSDKSEELFKAVVHAHSILTDEDQKLRYDRDLKIKGLHTYQPKKNCHIFKTKAKESQGASPTLGQSEAYHRQNKPYEQQPYGFGVGKKMTSSSKSKVPIFKSFNLKSYQRNHYYSSKKERKHGSPDIDSLFHETNGASKVRMTDAGKMDTNSQFQEIWEILGKNAYTHKSYSEDPNSCLGSALSDHEEEEEAGKQQQQQQQQQQQQQHYGMTSKSSSPDEEKKNNKEPKRESRVSPEENGEEETGHKQFKLPKTSTFSSGSHDSNLQSPFYNHEYRHYARSKFECKNQFRKSVSPIKEIPATTSANEGWNILRDIIEKLNISNVDDRNKDLLFRRDEIGDKNHSDSIDIENLSIKEPKGMKRRKKDDISLEELFQSLPREKDYFMMDAINDSLESINLFKKPKTTQSHEQGGTFAQAESNRAKFKPLLEQCGITPEILDLEIPEIPEFDAVADLETLKLNVQLFNNQCNKLKETIHQVSLQRLRADTQFSDMLTQKQSIMVWKTYLEFDKSLMDKLNILQERQMQVIKIFSERCDGKV

SEQ ID NO: 80 - DNA, 1752 bp, Saccharomyces cerevisiae
atgtcacaggtaatagaaccacaattagatagaacaacctattattccatattaggcttgacatcaaatgcgacttcctccgaagtacataaatcatatctaaaactggccagattacttcacccagataaaacaaaatctgataagtctgaggaattattcaaagctgtggtgcatgcacattcaattttaactgatgaagatcaaaaacttcgatatgatcgagatttgaaaatcaaaggtttacacacttaccagccgaagaaaaactgtcatattttcaagaccaaggcaaaggaatcacaaggggctagtcccacacttggtcaatcagaagcttatcataggcaaaataaaccttatgagcaacagccctacggtttcggtgtaggcaaaaaaatgacctcaagctctaagagtaaggttccgatattcaagtccttcaatttaaaaagctaccaacgaaaccactattattcatccaaaaaggaaaggaaacatggaagtcctgatattgattctttgttccatgaaaccaatggagcctcaaaagtaagaatgactgatgccggtaaaatggatacgaactctcagttccaagaaatatgggaaatattgggtaaaaatgcgtacacacataaatcttactctgaagatccaaattcatgtttgggatcagcactaagcgatcatgaagaagaagaagaagcaggaaaacaacaacagcaacagcagcaacaacagcaacagcagcaacattatggaatgacgtcgaagtctagcagtcctgatgaagaaaaaaaaaataataaagaaccgaaaagggaaagcagagtctctccagaggaaaatggcgaagaagaaacgggacacaaacaatttaaattgcccaagaccagtactttttctagtggatcccatgattcaaatttgcaatctcctttttacaatcatgagtatcgacattacgcaagaagtaaattcgaatgcaagaatcagtttagaaagtcagtttctcccattaaagagatacctgcaacaactagtgccaatgaaggatggaacattttgagagacattattgaaaaactcaatataagcaatgtagacgatcgaaataaagacttgctgtttcgtcgggatgaaataggtgataaaaatcacagcgactcaatcgacatagaaaatttatctatcaaagaacctaaagggatgaaaaggagaaagaaagatgatatatctttagaagaattgttccaatctttaccaagagaaaaagattattttatgatggatgcaattaatgactcgttagaatcaatcaatctttttaaaaagccgaagaccactcagagtcacgaacaaggtggaacttttgcccaagcagaaagtaatcgtgcaaaattcaaaccgttactagaacagtgtggaattacacccgagatcttagatttggaaataccagagattccggaatttgatgcagtggctgaccttgaaacattgaagcttaacgtgcagctgtttaataaccaatgtaacaaacttaaagaaacaatacatcaagtatcattacagcgcctgagagcagatacgcagttcagtgatatgttaacccaaaagcaaagtattatggtttggaaaacatacctagaatttgataaaagtttaatggacaaattgaacatcttacaagaaagacagatgcaggtcattaaaattttttccgaaagatgtgacggtaaagtataa

SEQ ID NO: 81 - protein, 172 aa, Saccharomyces cerevisiae
MSLVNSLTHYEILRIPSDATQDEIKKAYRNRLLNTHPDKLSKSIHDTVSNVTINKIQDAYKILSNIKTRREYDRLILENYKRQGFHNCGDGLDEFSLDDFSFDEDKLEFMMNCPRCQFVGGFHFSESLLDECIDNVDAMERSHSGYQLLTQCSACSLWLKVNFDIEEEQEGQ

SEQ ID NO: 82 - DNA, 519 bp, Saccharomyces cerevisiae
atgtcattggtgaattcgttaacacactacgaaattttaagaattccatcggatgcaacacaagatgaaatcaaaaaggcatataggaatcggttactaaatacgcaccccgataaactttctaaaagcatacatgatacggttagcaacgtcacaatcaataagattcaagatgcttataaaatactatcgaatataaaaactcgtcgcgaatatgataggttgatccttgaaaactataaacgccaaggatttcataattgtggtgatgggctggatgaattttccttagacgatttctcatttgatgaagataagctggagtttatgatgaattgtcctcgctgtcaatttgttggtggttttcattttagtgagagtttgttagatgaatgcattgataatgtagacgctatggaacggagtcattctggttatcaattattaacccaatgtagcgcatgcagcttatggctgaaggttaattttgacatcgaggaagagcaagaaggacaataa

SEQ ID NO: 83 - protein, 391 aa, Saccharomyces cerevisiae
MVKETEYYDILGIKPEATPTEIKKAYRRKAMETHPDKHPDDPDAQAKFQAVGEAYQVLSDPGLRSKYDQFGKEDAVPQQGFEDASEYFTAIFGGDGFKDWIGEFSLFKELNEATEMFGKEDEEGTAATETEKADESTDGGMVKHDTNKAESLKKDKLSKEQREKLMEMEKKRREDMMKQVDELAEKLNEKISRYLIAVKSNNLEEFTRKLDQEIEDLKLESFGLELLYLLARVYKTKANNFIMSKKTYGISKIFTGTRDNARSVKSAYNLLSTGLEAQKAMEKMSEVNTDELDQYERAKFESTMAGKALGVMWAMSKFELERKLKDVCNKILNDKKVPSKERIAKAKAMLFIAHKFASARRSPEEAEEARVFEELILGEQEKEHKKHTVAR

SEQ ID NO: 84 - DNA, 1176 bp, Saccharomyces cerevisiae
atggtaaaggagacggagtattatgatattttgggcatcaagcctgaggccacgcccactgaaatcaaaaaggcctatcgtagaaaggctatggaaacacatccggacaagcatcctgatgacccagatgctcaagcaaagtttcaagccgtaggcgaggcctaccaagtcttaagtgatccagggcttcgttccaagtatgaccagtttggtaaggaggatgctgttcctcagcaaggatttgaagatgcttctgaatactttacagcaatattcggtggtgatggcttcaaagattggattggagaattttctttgttcaaagagctaaacgaggcaacagaaatgtttggaaaggaagatgaggagggtacagcagccactgaaaccgaaaaagcagatgagagcactgatggtggaatggttaagcatgacactaataaagctgaatctttgaaaaaagataaattatcgaaggagcaaagagagaagctaatggaaatggagaaaaaaagacgggaagatatgatgaaacaagtcgacgagttggcagaaaaactgaacgaaaaaatctctaggtacttaattgctgtgaagtccaataacttggaggaatttacgcgaaaactagatcaagaaatcgaggatttgaaattagaaagttttggtctagagttattgtatttattggccagggtttacaagacaaaagcgaataattttatcatgtccaagaagacttacggaatttctaaaatattcactggtacacgcgacaatgctagatctgttaaatcagcatacaatttattgtctacaggcttagaagctcaaaaagccatggaaaaaatgagtgaagtcaatactgacgaactagaccaatatgaacgtgccaaatttgagtccacaatggctggtaaggcacttggtgtcatgtgggctatgtcgaaatttgaactggaaagaaaactaaaagacgtttgcaataagattctaaacgataaaaaggtcccttccaaggaacgtattgcaaaggcaaaagcaatgctgtttattgcccacaagtttgccagtgctagaaggtcaccagaagaagctgaagaagctagagtttttgaagagctaatcctaggtgagcaggagaaggaacacaaaaaacatactgtggccagataa

SEQ ID NO: 85 - protein, 283 aa, Saccharomyces cerevisiae
MPGHELEDVINQRLNLYDVLELPTPLDVHTIYDDLPQIKRKYRTLALKYHPDKHPDNPSIIHKFHLLSTATNILTNADVRPHYDRWLIEFLRKTNDIERNKLIQKLEESESSTIPTTTPHPDLLQIQRHGELLRKLKHFNLPYGDWKHLNTQDQENASQHPYYDCSTLRIVLDNFLQSNNKSNCLSHLRNQVFITLSANEIYDIYFSERNNYSKDDSIIIYTVFDTPITAQHVFRNWSSGNLIPTVKDISPLIPLHYYSDFNLETELNDDIARLVSNEPILLD

SEQ ID NO: 86 - DNA, 852 bp, Saccharomyces cerevisiae
atgccaggacacgaattggaagacgtaataaatcaacgtttgaacctatatgatgtattagaattaccgacccccctggacgtccataccatctacgatgatttgccccaaattaaacgcaaatacaggacccttgccctgaagtatcatcctgacaaacacccggacaatccatcaattatacacaaattccacttattatcgaccgcaactaatatcctcaccaatgcagacgtgagaccccattacgaccgctggttaattgagttcctacggaaaacaaacgacattgaaagaaataaacttatacaaaagctggaagaatctgaatcgagtacgatacccaccaccacaccacatcctgatttattgcaaatccaacgccacggcgagctactcaggaaactaaaacatttcaacttgccctatggtgactggaaacatctcaacacacaagaccaagaaaatgcttcgcaacatccgtattacgattgctctactttgagaattgtccttgacaacttcctgcaatcaaataataaatcaaactgcttatctcatttgcgcaatcaagtattcatcacgctaagtgctaatgaaatctacgacatctacttctctgaaagaaacaactactcgaaggatgattcaatcatcatatatactgtattcgatactcccatcacagcgcagcacgtattccgaaactggtcaagtgggaacctcatacccacggtcaaggatatttcgcccttgatcccgctacattactactctgattttaatttggagacggaactgaatgacgatattgcaagactggtctctaatgaacctatcctactcgactag

SEQ ID NO: 87 - protein, 168 aa, Saccharomyces cerevisiae
MSSQSNTGNSIEAPQLPIPGQTNGSANVTVDGAGVNVGIQNGSQGQKTGMDLYFDQALNYMGEHPVITGFGAFLTLYFTAGAYKSISKGLNGGKSTTAFLKGGFDPKMNSKEALQILNLTENTLTKKKLKEVHRKIMLANHPDKGGSPFLATKINEAKDFLEKRGISK

SEQ ID NO: 88 - DNA, 507 bp, Saccharomyces cerevisiae
atgagttctcaaagtaatactggtaattctattgaggcaccacaactacccattcctggtcaaactaatggctctgcgaacgttactgttgatggagctggtgttaatgtcggtatccagaatggttcgcagggtcaaaagaccggaatggacctttattttgatcaagctttgaactacatgggagaacatcctgtgataacaggttttggggcctttttaactttatattttacagccggtgcatataaatcaatatcgaagggacttaacggtggaaaatccactactgccttcttgaaaggcggatttgacccgaaaatgaattctaaagaggctctacagattttgaatttgacagaaaatacattgactaaaaaaaagttgaaagaggttcataggaaaattatgttagctaatcatcctgacaaaggtggttctccatttttggccactaagataaacgaagctaaggactttttggaaaaaaggggtattagcaaataa

SEQ ID NO: 89 - protein, 184 aa, Saccharomyces cerevisiae
MLKYLVQRRFTSTFYELFPKTFPKKLPIWTIDQSRLRKEYRQLQAQHHPDMAQQGSEQSSTLNQAYHTLKDPLRRSQYMLKLLRNIDLTQEQTSNEVTTSDPQLLLKVLDIHDELSQMDDEAGVKLLEKQNKERIQDIEAQLGQCYNDKDYAAAVKLTVELKYWYNLAKAFKDWAPGKQLEMNH

SEQ ID NO: 90 - DNA, 555 bp, Saccharomyces cerevisiae
atgttgaaatacttggttcaacgaagattcacttctacattttacgagctgttcccaaagaccttccccaaaaagctacccatttggactatcgatcaatccagattaaggaaggagtataggcaattacaagcacagcaccatccagacatggcccaacaaggtagtgaacagtcatcaactcttaatcaagcttaccatactctcaaagatccccttagaaggtcacaatatatgctaaaactcttgcgcaatatcgatttgacgcaagaacagacctcaaatgaagtaactaccagtgatccacagttactattgaaagttctagacatccatgatgaattatcccagatggacgacgaagctggtgtgaagctgcttgaaaagcaaaacaaggaaagaattcaagatattgaagcccagttgggacaatgctacaatgacaaggattacgccgccgcagtgaagttgaccgtggagctaaagtactggtacaacttggccaaggcattcaaagactgggctccaggaaaacaattggaaatgaatcactaa

SEQ ID NO: 91 - protein, 301 aa, Saccharomyces cerevisiae
MLHHKFVYPFLFKWHLSCVEKCPPQITFIAKYATANDKNGNRKLTIRDEQWPELADPTPYDIFGIPKAGSGNPKLDKKSLKKKYHRYVKLYHPDHSDNIQIFSSEKVTNSDSKSPLLLTSSEKLHRFKVISQAYDILCDPKKKIVYDTTRQGWTTSYSPRSNVNTENYQYAGSYGYHSNAQYEYWNAGTWEDANSMKNERIQENINPWTVIGIICGLAICIEGTALLAKIQESLSKAEFTHDESGLHLIQSYTNYGLDTDKFSRLRRFLWFRTWGLYKSKEDLDREAKINEEMIRKLKAAK

SEQ ID NO: 92 - DNA, 906 bp, Saccharomyces cerevisiae
atgctacaccataagttcgtatacccatttttattcaagtggcacttatcatgtgtagaaaagtgtcccccacaaatcacttttatagctaagtatgctacagcgaacgataaaaatggcaatagaaaacttacgataagggatgaacaatggcctgagttggcagatccaactccctatgatatttttggcattccaaaggccggatctggaaatcctaaactggacaagaagtcgttaaaaaaaaaatatcatcgttatgtaaaattgtaccaccctgaccattccgataacattcaaatatttagctcagaaaaggttaccaacagtgatagtaaatcaccgctgctgctaacatcaagcgaaaaactacatagatttaaagtcatctctcaagcatatgatattctttgtgacccaaagaaaaagatcgtatatgacacaacgaggcaaggctggaccacatcgtattcaccacgttctaacgttaatactgaaaattaccaatatgccggctcttatggctaccactctaacgcgcagtatgaatactggaacgctgggacttgggaagacgcaaatagcatgaaaaacgaaagaattcaagaaaacatcaacccatggaccgttattggcataatttgtggcctagctatatgcatcgaagggactgcgttgttagccaaaatccaggagtctctgagcaaggccgaatttactcatgacgaaagtggattacatttgattcagtcatacacgaattatggtcttgatactgacaaattttccagattgaggcggttcttatggtttagaacttggggactttacaagtcgaaagaggatttagatagagaagccaagatcaatgaagaaatgatacgcaaactgaaagcagctaaatga

SEQ ID NO: 93 - protein, 224 aa, Saccharomyces cerevisiae
MSFTEDQEKIALEILSKDKHEFYEILKVDRKATDSEIKKAYRKLAIKLHPDKNSHPKAGEAFKVINRAFEVLSNEEKRSIYDRIGRDPDDRQMPSRGAASGFRGSAGGSPMGGGFEDMFFNSRFGGQRAGPPEDIFDFLFNAGGSPFGASPFGPSASTFSFGGPGGFRVYTNNRGGSPFMRQQPRSRQQQQQAEENAVNSQLKNMLVLFIIFIVLPMIKDYLFS

SEQ ID NO: 94 - DNA, 675 bp, Saccharomyces cerevisiae
atgtctttcactgaggatcaagaaaaaatcgcgctagaaatactgtcaaaagacaagcatgagttttacgaaattttgaaggtagataggaaagccacagatagtgagatcaagaaggcatacagaaaactagcaatcaaattgcatcctgataaaaactctcatccaaaagcgggagaagctttcaaagtaattaatagggcatttgaagtactaagcaatgaggaaaagcgcagtatttatgacaggataggtagggatcctgacgatagacaaatgccatccagaggtgctgcttcagggttccgaggaagtgcaggtgggtctccaatgggtggcggatttgaagacatgtttttcaattcacgtttcggtggtcaaagagctggaccaccagaggacatattcgactttttgttcaacgcaggcggcagcccattcggcgcttcaccatttgggccttctgcttccactttttcatttggaggccccggtggtttcagagtttatactaataatcgtggtggctcaccgttcatgcgtcaacaaccccgctcaagacagcagcaacaacaagcagaagaaaatgcagtgaattcgcaattaaaaaatatgctcgttcttttcatcatctttattgttcttcctatgattaaagattacctgtttagttaa

SEQ ID NO: 95 - protein, 295 aa, Saccharomyces cerevisiae
MNGYWKPALVVLGLVSLSYAFTTIETEIFQLQNEISTKYGPDMNFYKFLKLPKLQNSSTKEITKNLRKLSKKYHPDKNPKYRKLYERLNLATQILSNSSNRKIYDYYLQNGFPNYDFHKGGFYFSRMKPKTWFLLAFIWIVVNIGQYIISIIQYRSQRSRIENFISQCKQQDDTNGLGVKQLTFKQHEKDEGKSLVVRFSDVYVVEPDGSETLISPDTLDKPSVKNCLFWRIPASVWNMTFGKSVGSAGKEEIITDSKKYDGNQTKKGNKVKKGSAKKGQKKMELPNGKVIYSRK

SEQ ID NO: 96 - DNA, 888 bp, Saccharomyces cerevisiae
atgaacggttactggaaacctgcgttggttgtcctgggattggtatctctatcatatgcttttaccaccattgaaacagaaattttccaattacaaaatgaaataagtacgaaatatggcccagatatgaacttctacaagttcttgaagttacctaaactgcagaattctagtacaaaggagattacaaaaaacttaagaaagctatccaagaagtaccatccggataagaaccctaaataccgtaaattgtatgaaaggttaaacctcgctactcaaattctttcaaacagctctaatcgtaagatttatgattattatctacagaatggctttccaaactatgatttccataagggtggtttttatttttccagaatgaagcctaagacttggttcctgctggcctttatttggatagtcgttaatattgggcagtatatcatttctattattcaatatcgttctcaaagatcaagaattgaaaacttcatcagtcagtgtaaacaacaggatgataccaatggactaggcgtaaaacaactaacgtttaaacaacatgaaaaggatgagggtaaaagtttggttgtaaggtttagcgatgtctatgttgtagagcctgatggaagtgaaacactaatttcgccagataccttggataaaccttcagtaaagaactgtttgttttggagaatacctgcttcggtttggaacatgacgtttggcaaatctgttggtagcgcaggaaaagaagaaataataacggatagtaaaaagtatgatggtaaccaaacaaaaaaggggaacaaagtaaaaaagggttctgcaaagaaaggccaaaagaaaatggaattgcctaacggtaaagtgatctattcacgtaaatga

SEQ ID NO: 97 - protein, 228 aa, Saccharomyces cerevisiae
MRAFSAATVRATTRKSFIPMAPRTPFVTPSFTKNVGSMRRMRFYSDEAKSEESKENNEDLTEEQSEIKKLESQLSAKTKEASELKDRLLRSVADFRNLQQVTKKDIQKAKDFALQKFAKDLLESVDNFGHALNAFKEEDLQKSKEISDLYTGVRMTRDVFENTLRKHGIEKLDPLGEPFDPNKHEATFELPQPDKEPGTVFHVQQLGFTLNDRVIRPAKVGIVKGEEN

SEQ ID NO: 98 - DNA, 687 bp, Saccharomyces cerevisiae
atgagagctttttcagcagccaccgttagggccacaactaggaagtcgttcatcccaatggcaccaagaactccttttgtgactccatcatttacaaagaatgtaggctcaatgagaagaatgagattttattctgatgaagccaaaagtgaagaatccaaagaaaacaatgaagatttgactgaagagcaatcagaaatcaagaaattagagagccagttaagcgcgaagactaaagaagcttctgaactcaaggacagattattaagatctgtggcagatttcagaaatttacaacaagtcacaaagaaggatattcagaaagctaaggactttgctttacagaagtttgcaaaggatttattggaatctgtagataactttggtcatgctttgaatgcttttaaagaggaagacttacaaaagtccaaggaaattagtgatttgtatacaggggttagaatgacaagagatgtttttgaaaacaccctaagaaagcacggtattgaaaaattagacccattgggagaaccatttgatccaaataaacacgaagcaacgttcgagttgccacaacctgataaggaaccgggtactgttttccatgtacaacaattaggtttcaccttgaatgacagagttatcagaccagcaaaagtcggaattgttaagggcgaagagaactaa

SEQ ID NO: 99 - protein, 290 aa, Saccharomyces cerevisiae
MEKLLQWSIANSQGDKEAMARAGQPDPKLLQQLFGGGGPDDPTLMKESMAVIMNPEVDLETKLVAFDNFEMLIENLDNANNIENLKLWEPLLDVLVQTKDEELRAAALSIIGTAVQNNLDSQNNFMKYDNGLRSLIEIASDKTKPLDVRTKAFYALSNLIRNHKDISEKFFKLNGLDCIAPVLSDNTAKPKLKMRAIALLTAYLSSVKIDENIISVLRKDGVIESTIECLSDESNLNIIDRVLSFLSHLISSGIKFNEQELHKLNEGYKHIEPLKDRLNEDDYLAVKYVL

SEQ ID NO: 100 - DNA, 873 bp, Saccharomyces cerevisiae
atggaaaagctattacagtggtctattgcgaattctcaaggggacaaagaagctatggctagggccggccaacctgatcctaaattgctacagcagttattcggtggtggtggtcctgacgatccaaccttaatgaaagaatccatggctgttattatgaatccggaggttgacttagaaacaaaactcgttgcatttgacaactttgaaatgttgattgagaacttagataatgctaataatatcgaaaatttaaaactgtgggagccattgttggatgttcttgttcagacgaaggatgaagaactacgtgctgctgctttatccattattggaacggctgtgcaaaacaacttggattcgcaaaataatttcatgaaatacgacaatggtctgcgaagccttatcgaaatagctagtgacaagacaaagccactcgacgtgagaacaaaagctttttacgcactatctaatctaataagaaaccacaaagatatctcagaaaagtttttcaaattaaatgggctcgactgcatagcacctgtattaagtgataacaccgccaaaccaaaactgaaaatgagagccattgccttattgaccgcatatttgtcatctgttaagattgatgaaaatataatcagtgtgctgagaaaggatggagtaattgaaagtacgattgagtgcttgtctgacgagagtaacttgaacatcatagatagagttctgtcttttctctctcacctgatatcttccggaataaaatttaatgaacaggaattgcacaaattgaacgaaggttacaaacatatcgagcctctaaaggacagacttaatgaagacgattatttagccgtaaagtatgtattatga

SEQ ID NO: 101 - DNA, 40 bp, Artificial Sequence
  Primer HO 5′ ForNotIBbsI
gcatgcggccgcccgaagaccctacacagggcttaagggc

SEQ ID NO: 102 - DNA, 35 bp, Artificial Sequence
  Primer HO 5′ RevBsiWIMluI
ccacgcgtcgtacgggattgctgcttatgaggata

SEQ ID NO: 103 - DNA, 37 bp, Artificial Sequence
  Primer HO 3′ ForMluIEcoRI
acgcgtgaattcaaaaagggaacccgtatatttcagc

SEQ ID NO: 104 - DNA, 39 bp, Artificial Sequence
  Primer HO 3′ RevBbsIClaI
tatcgatagtcttcctaatatacacattttagcagatgc

SEQ ID NO: 105 - DNA, 102 bp, Artificial Sequence
  Primer pBST HO Poly For
gcatgcatacgcgtcacgcatgtgcctcagcggccggccggcgccgggccccggaccgcctgcaggctcgagttaattaagtttaaacgaattcgcatgcat

SEQ ID NO: 106 - DNA, 102 bp, Artificial Sequence
  Primer pBST HO Poly Rev
atgcatgcgaattcgtttaaacttaattaactcgagcctgcaggcggtccggggcccggcgccggccggccgctgaggcacatgcgtgacgcgtatgcatgc

SEQ ID NO: 107 - DNA, 62 bp, Artificial Sequence
  Primer Ycplac33 Poly For
ctagattggatccctagtctaggtttaaactagcgattcacctaggtgctaggaattctagc

SEQ ID NO: 108 - DNA, 62 bp, Artificial Sequence
  Primer Ycplac33 Poly Rev
gctagaattcctagcacctaggtgaatcgctagtttaaacctagactagggatccaatctag

SEQ ID NO: 109 - DNA, 69 bp, Artificial Sequence
  Primer LHS1forOverlap
cacaatatttcaagctataccaagcatacaatcaactatctcatatacaatgcgaaacgttttaaggct

SEQ ID NO: 110 - DNA, 34 bp, Artificial Sequence
  Primer LHS1revBbvCI
gcatgctgagggtgccactataatattaatgtgc

SEQ ID NO: 111 - DNA, 71 bp, Artificial Sequence
  Primer SLS1forOverlap
caccaacacacacaaaaaacagtacttcactaaatttacacacaaaacaaaatggtccggattcttcccat

SEQ ID NO: 112 - DNA, 31 bp, Artificial Sequence
  Primer SLS1revNarI
gcatggcgccccacggcagggcagttggcac

SEQ ID NO: 113 - DNA, 70 bp, Artificial Sequence
  Primer JEM1forOverlap
cagatcatcaaggaagtaattatctactttttacaacaaatataaaacaatgatactgatctcgggatac

SEQ ID NO: 114 - DNA, 33 bp, Artificial Sequence
  Primer JEM1revRsrII
cgatcggtccgagggaaataaggcagatcaaag

SEQ ID NO: 115 - DNA, 72 bp, Artificial Sequence
  Primer SCJ1forOverlap
cacgcttactgcttttttcttcccaagatcgaaaatttactgaattaacaatgattccaaaattatatatac

SEQ ID NO: 116 - DNA, 29 bp, Artificial Sequence
  Primer SCJ1revXhoI
gcatctcgaggactttgagacctgtgatc

SEQ ID NO: 117 - DNA, 38 bp, Artificial Sequence
  Primer ADH1promForAleI
cgatcaccgatgtggttgtttccgggtgtacaatatgg

SEQ ID NO: 118 - DNA, 71 bp, Artificial Sequence
  Primer ADH1promRevOverlap
cctatagcaacaaaagctgttaaaaataaaagccttaaaacgtttcgcattgtatatgagatagttgattg

SEQ ID NO: 119 - DNA, 32 bp, Artificial Sequence
  Primer PGK1promForPspOMI
gcatgggcccagattcctgacttcaactcaag

SEQ ID NO: 120 - DNA, 73 bp, Artificial Sequence
  Primer PGK1promRevOverlap
ggcaaaataacgctatacactaaaagacagtatcccgagatcagtatcattgttttatatttgttgtaaaaac

SEQ ID NO: 121 - DNA, 33 bp, Artificial Sequence
  Primer TDH1promForFseI
gcatggccggccaccatatggaggataagttgg

SEQ ID NO: 122 - DNA, 71 bp, Artificial Sequence
  Primer TDH1promRevOverlap
ctaatttcgaagatagggcgctcaaaattatgggaagaatccggaccattttgttttgtgtgttttaaatc

SEQ ID NO: 123 - DNA, 36 bp, Artificial Sequence
  Primer TEF1promForSbfI
cggtagtacctgcaggaagcaacaggcgcgttggac

SEQ ID NO: 124 - DNA, 78 bp, Artificial Sequence
  Primer TEF1promRevOverlap
ggcaacaacaataaagatagtatcaaatgtatatataattttggaatcattttgtaattaaaacttagattagattgc

SEQ ID NO: 125 - DNA, 32 bp, Artificial Sequence
  Primer URA3forPac1
ctagagttaattaagtttcaattcaattcatc

SEQ ID NO: 126 - DNA, 32 bp, Artificial Sequence
  Primer URA3revPme1
gcctgagtttaaacgttttctttccaattttt

SEQ ID NO: 127 - DNA, 40 bp, Artificial Sequence; identical to SEQ ID NO: 101
  Primer A01

SEQ ID NO: 128 - DNA, 35 bp, Artificial Sequence; identical to SEQ ID NO: 102
  Primer A02

SEQ ID NO: 129 - DNA, 37 bp, Artificial Sequence; identical to SEQ ID NO: 103
  Primer A03

SEQ ID NO: 130 - DNA, 39 bp, Artificial Sequence; identical to SEQ ID NO: 104
  Primer A04

SEQ ID NO: 131 - DNA, 102 bp, Artificial Sequence; identical to SEQ ID NO: 105
  Primer A05

SEQ ID NO: 132 - DNA, 102 bp, Artificial Sequence; identical to SEQ ID NO: 106
  Primer A06

SEQ ID NO: 133 - DNA, 35 bp, Artificial Sequence
  Primer A07
ctaggtaacttaattaagggtaagctgccacagca

SEQ ID NO: 134 - DNA, 34 bp, Artificial Sequence
  Primer A08
ctacgtactctagatgttaattcagtaaattttc

SEQ ID NO: 135 - DNA, 30 bp, Artificial Sequence
  Primer A09
ctagactctagatctctgcttttgtgcgcg

SEQ ID NO: 136 - DNA, 34 bp, Artificial Sequence
  Primer A10
catgctacgtttaaacgatgatcatatgatacac

SEQ ID NO: 137 - DNA, 32 bp, Artificial Sequence; identical to SEQ ID NO: 125
  Primer A11

SEQ ID NO: 138 - DNA, 32 bp, Artificial Sequence; identical to SEQ ID NO: 126
  Primer A12

SEQ ID NO: 139 - DNA, 62 bp, Artificial Sequence; identical to SEQ ID NO: 107
  Primer A13

SEQ ID NO: 140 - DNA, 62 bp, Artificial Sequence; identical to SEQ ID NO: 108
  Primer A14

SEQ ID NO: 141 - DNA, 69 bp, Artificial Sequence; identical to SEQ ID NO: 109
  Primer C01

SEQ ID NO: 142 - DNA, 34 bp, Artificial Sequence; identical to SEQ ID NO: 110
  Primer C02

SEQ ID NO: 143 - DNA, 30 bp, Artificial Sequence
  Primer C03
ctagatctctagaatggtccggattcttcc

SEQ ID NO: 144 - DNA, 31 bp, Artificial Sequence; identical to SEQ ID NO: 112
  Primer C04

SEQ ID NO: 145 - DNA, 30 bp, Artificial Sequence
  Primer C05
ctagatctctagaatgatactgatctcggg

SEQ ID NO: 146 - DNA, 33 bp, Artificial Sequence; identical to SEQ ID NO: 114
  Primer C06

SEQ ID NO: 147 - DNA, 72 bp, Artificial Sequence; identical to SEQ ID NO: 115
  Primer C07

SEQ ID NO: 148 - DNA, 29 bp, Artificial Sequence
  Primer C08
gcatctcgaggactttgagacctgtgatc

SEQ ID NO: 149 - DNA, 38 bp, Artificial Sequence; identical to SEQ ID NO: 117
  Primer C09

SEQ ID NO: 150 - DNA, 71 bp, Artificial Sequence; identical to SEQ ID NO: 118
  Primer C10

SEQ ID NO: 151 - DNA, 32 bp, Artificial Sequence; identical to SEQ ID NO: 119
  Primer C11

SEQ ID NO: 152 - DNA, 32 bp, Artificial Sequence
  Primer C12
gatctagtctagatgttttatatttgttgtaa

SEQ ID NO: 153 - DNA, 33 bp, Artificial Sequence; identical to SEQ ID NO: 121
  Primer C13

SEQ ID NO: 154 - DNA, 34 bp, Artificial Sequence
  Primer C14
acctagtctagatttgttttgtgtgtaaatttag

SEQ ID NO: 155 - DNA, 36 bp, Artificial Sequence; identical to SEQ ID NO: 123
  Primer C15

SEQ ID NO: 156 - DNA, 78 bp, Artificial Sequence; identical to SEQ ID NO: 124
  Primer C16

SEQ ID NO: 157 - DNA, 34 bp, Artificial Sequence
  Primer C17
ctagtctctagaatggaaatgactgattttgaac

SEQ ID NO: 158 - DNA, 31 bp, Artificial Sequence
  Primer C18
ctagtctagatcatgaagtgatgaagaaatc

SEQ ID NO: 159 - DNA, 23 bp, Artificial Sequence
  ACT1 Forward Primer
cccagaagctttgttccatcctt

SEQ ID NO: 160 - DNA, 28 bp, Artificial Sequence
  ACT1 Reverse Primer
atgatggagttgtaagtagtttggtcaa

SEQ ID NO: 161 - DNA, 20 bp, Artificial Sequence
  ACT1 Probe
cagattccaaacccaaaaca

SEQ ID NO: 162 - DNA, 25 bp, Artificial Sequence
  LHS1 Forward Primer
acactactcagcccgttacaataga

SEQ ID NO: 163 - DNA, 25 bp, Artificial Sequence
  LHS1 Reverse Primer
gtaaactttgcaccacctagatgtg

SEQ ID NO: 164 - DNA, 23 bp, Artificial Sequence
  LHS1 Probe
atttgaaggatatgggtataatc

SEQ ID NO: 165 - DNA, 29 bp, Artificial Sequence
  SIL1 Forward Primer
gacatgtacgaaaatgacgatacaaatct

SEQ ID NO: 166 - DNA, 19 bp, Artificial Sequence
  SIL1 Reverse Primer
tcgtttgcccactcttgca

SEQ ID NO: 167 - DNA, 17 bp, Artificial Sequence
  SIL1 Probe
tttgacgaccaattctc

SEQ ID NO: 168 - DNA, 17 bp, Artificial Sequence
  SCJ1 Forward Primer
ggcgcaggtggattcca

SEQ ID NO: 169 - DNA, 18 bp, Artificial Sequence
  SCJ1 Reverse Primer
cgccaggacctccatgac

SEQ ID NO: 170 - DNA, 20 bp, Artificial Sequence
  SCJ1 Probe
catattcgaacggatgtttc

SEQ ID NO: 171 - DNA, 18 bp, Artificial Sequence
  JEM1 Forward Primer
cctctccacgcacatcga

SEQ ID NO: 172 - DNA, 25 bp, Artificial Sequence
  JEM1 Reverse Primer
tgcttgtcgaggattgtttcgtaat

SEQ ID NO: 173 - DNA, 19 bp, Artificial Sequence
  JEM1 Probe
tcgttagctgctgctatca

SEQ ID NO: 174 - DNA, 19 bp, Artificial Sequence
  HAC1 Forward Primer
gaagacgcgttgacttgca

SEQ ID NO: 175 - DNA, 25 bp, Artificial Sequence
  HAC1 Reverse Primer
gaaatccctgtactcgtcaagagaa

SEQ ID NO: 176 - DNA, 18 bp, Artificial Sequence
  HAC1 Probe
ccacgacgcttttgttgc

SEQ ID NO: 177 - DNA, 26 bp, Artificial Sequence
  HAC1i Forward Primer
acaattcaattgatcttgacaattgg

SEQ ID NO: 178 - DNA, 25 bp, Artificial Sequence
  HAC1i Reverse Primer
tcaattcaaatgaatcaaacctgac

SEQ ID NO: 179 - DNA, 17 bp, Artificial Sequence
  HAC1i Probe
cgtaatccagaagcgca

SEQ ID NO: 180 - DNA, 157 bp, Artificial Sequence
  Oligonucleotide linker sense for construction of pDB2283
gagtccaattagcttcatcgccaataaaaaaacaagctaaacctaattctaacaagcaaagatgaagtgggtaagcttaacctaattctaacaagcaaagatgcttttgcaagccttccttttccttttggctggttttgcagccaaaatatctgca

SEQ ID NO: 181 - DNA, 161 bp, Artificial Sequence
  Single stranded oligonucleotide 1 for pDB2283
ttaagagtccaattagcttcatcgccaataaaaaaacaagctaaacctaattctaacaagcaaagatgaagtgggtaagcttaacctaattctaacaagcaaagatgcttttgcaagccttccttttccttttggctggttttgcagccaaaatatctgca

SEQ ID NO: 182 - DNA, 157 bp, Artificial Sequence
  Single stranded oligonucleotide 2 for pDB2283
tgcagatattttggctgcaaaaccagccaaaaggaaaaggaaggcttgcaaaagcatctttgcttgttagaattaggttaagcttacccacttcatctttgcttgttagaattaggtttagcttgtttttttattggcgatgaagctaattggactc

SEQ ID NO: 183 - DNA, 320 bp, Artificial Sequence
  Synthetic phosphorylated oligonucleotide linker sense for construction of pDB2284
ttaagagtccaattagcttcatcgccaataaaaaaacaaactaaacctaattctaacaagcaaagatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtaagcttggataaaaga

SEQ ID NO: 184 - DNA, 316 bp, Artificial Sequence
  Synthetic phosphorylated oligonucleotide linker anti sense (pDB2283)
tcttttatccaagcttaccccttcttctttagcagcaatgctggcaatagtagtatttataaacaataacccgttatttgtgctgttggaaaatggcaaaacagcaacatcgaaatccccttctaaatctgagtaaccgatgacagcttcagccggaatttgtgccgtttcatcttctgttgtagtgttgactggagcagctaatgcggaggatgctgcgaataaaactgcagtaaaaattgaaggaaatctcatctttgcttgttagaattaggtttagtttgtttttttattggcgatgaagctaattggactc

SEQ ID NO: 185 - protein, 85 aa, Saccharomyces cerevisiae
MRFPSIFTAVLFAASSALAAPVNTTTEDETAQIPAEAVIGYSDLEGDFDVAVLPFSNSTNNGLLFINTTIASIAAKEEGVSLDKR

SEQ ID NO: 186 - DNA, 98 bp, Artificial Sequence
  Complimentary SS oligo 1
ttaagagtccaattagcttcatcgccaataaaaaaacaaactaaacctaattctaacaagcaaagatgagatttccttcaatttttactgcagtttta

SEQ ID NO: 187 - DNA, 110 bp, Artificial Sequence
  Complimentary SS oligo 2
ttcgcagcatcctccgcattagctgctccagtcaacactacaacagaagatgaaacggcacaaattccggctgaagctgtcatcggttactcagatttagaaggggattt

SEQ ID NO: 188 - DNA, 112 bp, Artificial Sequence
  Complimentary SS oligo 3
cgatgttgctgttttgccattttccaacagcacaaataacgggttattgtttataaatactactattgccagcattgctgctaaagaagaaggggtaagcttggataaaaga

SEQ ID NO: 189 - DNA, 102 bp, Artificial Sequence
  Complimentary SS oligo 4
tcttttatccaagcttaccccttcttctttagcagcaatgctggcaatagtagtatttataaacaataacccgttatttgtgctgttggaaaatggcaaaac

SEQ ID NO: 190 - DNA, 110 bp, Artificial Sequence
  Complimentary SS oligo 5
agcaacatcgaaatccccttctaaatctgagtaaccgatgacagcttcagccggaatttgtgccgtttcatcttctgttgtagtgttgactggagcagctaatgcggagg

SEQ ID NO: 191 - DNA, 104 bp, Artificial Sequence
  Complimentary SS oligo 6
atgctgcgaataaaactgcagtaaaaattgaaggaaatctcatctttgcttgttagaattaggtttagtttgtttttttattggcgatgaagctaattggactc

SEQ ID NO: 192 - DNA, 77 bp, Artificial Sequence
  SINK1
gtaccaagctttatttcccttctttttctctttagctcggcttattccaggagcttggataaaagagcacccgcccg

SEQ ID NO: 193 - DNA, 78 bp, Artificial Sequence
  SINK2
gtgaccgggcgggtgctcttttatccaagctcctggaataagccgagctaaagagaaaaagaagggaaataaagcttg

SEQ ID NO: 194 - DNA, 450 bp, Artificial Sequence
  Human GMCSF with incorporated N-terminal Met codon
aagcttacctgccatggcacccgcccggtcacccagccccagcacgcagccctgggagcatgtgaatgccatccaggaggcccggcgtctcctgaacctgagtagagacactgctgctgagatgaatgaaacagtagaagtgatatcagaaatgtttgacctccaggagccgacttgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggacttcctgcttgtcatcccctttgactgctgggagccagtccaggagtgataaggatccgaattcgtaatcatggtcatagctgtttcctgtgtgaaatt

SEQ ID NO: 195 - protein, 128 aa, Artificial Sequence
  Human GMCSF with incorporated N-terminal Met codon
MAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE